(19) United States
(12) Patent Application Publication
(10) Pub. No.: US 2010/0256092 A1
(43) Pub. Date: Oct. 7, 2010

(54) BORON-CONTAINING SMALL MOLECULES

(75) Inventors: Yi Xia, Palo Alto, CA (US); Michael Richard Kevin Alley, Santa Clara, CA (US); Yasheen Zhou, Moraga, CA (US); Vincent S. Hernandez, Watsonville, CA (US); Jacob J. Plattner, Berkeley, CA (US); Charles Z. Ding, Foster City, CA (US); Kathy Cao, Sunnyvale, CA (US); Yong-Kang Zhang, San Jose, CA (US); Andrew Benowitz, Brentford (GB); Tsutomu Akama, Sunnyvale, CA (US); Jessica Sligar, Cary, NC (US); Guofeng Jia, Edmonton (CA); Ligong Ou, Edmonton (CA); Neerja Saraswat, Edmonton (CA); Sreekanth Ramachandran, Edmonton (CA); Chris Diaper, Edmonton (CA); Yanchen Zhang, Union City, CA (US); Goverdhan Reddy Banda, Edmonton (CA); James A. Nieman, Sherwood Park (CA); Mehdi Keramane, Edmonton (CA); Rahim Mohammed, Edmonton (CA); Rajendra Subedi, Edmonton (CA); Hong Liang, Edmonton (CA); Rajeshwar Singh, Edmonton (CA)

Correspondence Address: MORGAN, LEWIS & BOCKIUS LLP (SF), One Market, Spear Street Tower, Suite 2800, San Francisco, CA, 94105, US

(73) Assignees: Anacor Pharmaceuticals, Inc., Palo Alto, CA (US); GlaxoSmithKline, Research Triangle, NC (US)

(21) Appl. No.: 12/464,829

(22) Filed: May 12, 2009

Related U.S. Application Data
(60) Provisional application No. 61/052,604, filed on May 12, 2008.
(60) Provisional application No. 61/138,484, filed on Dec. 17, 2008.

(51) Int. Cl. A61K 31/69; C07F 5/02; A61P 31/04; A01N 55/08; A01P 1/00; C12N 9/99

(52) U.S. Cl. 514/64; 549/213; 544/229; 544/69; 548/110; 546/13; 435/184

(57) ABSTRACT

This invention relates to, among other items, 6-substituted benzoxaborole compounds and their use for treating bacterial infections.

### CROSS-REFERENCE TO RELATED APPLICATIONS

This application claims the benefit of U.S. Provisional Pat. App. No. 61/052,604, filed May 12, 2008 and U.S. Provisional Pat. App. No. 61/138,484, filed Dec. 17, 2008, each of which is incorporated by reference in its entirety for all purposes.

### BACKGROUND OF THE INVENTION

The global rise of bacteria and other microorganisms resistant to antibiotics and antimicrobials in general, poses a major threat. Deployment of massive quantities of antimicrobial agents into the ecosphere during the past 60 years has introduced a powerful selective pressure for the emergence and spread of antimicrobial-resistant pathogens. Thus, there is a need to discover new broad spectrum antimicrobials, such as antibiotics, useful in combating microorganisms, especially those with multidrug-resistance. There is also a need to discover compounds which are useful in inhibiting or deactivating the resistance mechanisms of microorganisms, such as beta-lactamase enzymes.

Boron-containing molecules, such as 1-hydroxy-1,3-dihydro-benzo[c][1,2]oxaborole (also sometimes known as 1-hydroxy-benzo[c][1,2]oxaborole or oxaboroles or cyclic boronic esters), useful as antimicrobials have been described previously, such as in U.S. patent application Ser. Nos. 12/142,692; 11/505,591 and 11/357,687. Generally speaking, a 1-hydroxy-1,3-dihydro-benzo[c][1,2]oxaborole has the following structure and substituent numbering system:

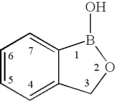

It has been discovered that certain classes of 1-hydroxy-1,3-dihydro-benzo[c][1,2]oxaboroles which are substituted at the 6-position with an unsubstituted or monosubstituted aryloxy, heteroaryloxy, cycloalkoxy or heterocycloalkoxy moiety are surprisingly effective beta-lactamase inhibitors. It has also been discovered that certain classes of 1-hydroxy-1,3-dihydro-benzo[c][1,2]oxaboroles which are substituted at the 6-position with an aryloxy, heteroaryloxy, cycloalkoxy or heterocycloalkoxy moiety, and are also substituted at the 3-position, are surprisingly effective beta-lactamase inhibitors. It has also been discovered that certain classes of 1-hydroxy-1,3-dihydro-benzo[c][1,2]oxaboroles which are substituted at the 6-position with an unsubstituted or monosubstituted aryl or heteroaryl sulfonamide moiety are surprisingly effective antibacterials. This, and other uses of these 1-hydroxy-1,3-dihydro-benzo[c][1,2]oxaboroles are described herein.

### SUMMARY OF THE INVENTION

In a first aspect, the invention provides a compound having a structure according to the formula:

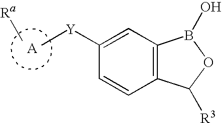

wherein A is a member selected from cycloalkyl, heterocycloalkyl, aryl and heteroaryl; Y is a member selected from O and —S(O)₂NH— wherein the sulfur in —S(O)₂NH— is covalently attached to A; R³ is a member selected from H, cyano and substituted alkyl; R^{a} is a member selected from H, —OR¹⁰, —NR¹⁰R¹¹, —SR¹⁰, —S(O)R¹⁰, —S(O)₂R¹⁰, —S(O)₂NR¹⁰R¹¹, —C(O)R¹⁰, —C(O)OR¹⁰, —C(O)NR¹⁰R¹¹, nitro, cyano, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, wherein each R¹⁰ and each R¹¹ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, with the proviso that R¹⁰ and R¹¹, together with the nitrogen to which they are attached, are optionally combined to form a 5- to 7-membered substituted or unsubstituted heterocycloalkyl ring; with the proviso that when Y is O, R³ is a member selected from cyano and substituted alkyl; with the proviso that when Y is —S(O)₂NH—, R³ is H, and R^{a} is not H or unsubstituted alkyl or halosubstituted alkyl, and salts thereof.

In another aspect, the invention provides a combination comprising: a) a compound of the invention; and b) a therapeutically active agent.

In another aspect, the invention provides a pharmaceutical formulation comprising: a) a compound of the invention or a combination of the invention; and b) a pharmaceutically acceptable excipient.

In another aspect, the invention provides a method of treating a bacterial infection comprising: administering to an animal suffering from said infection an effective amount of a compound of the invention, and an effective amount of an antibiotic, wherein said antibiotic comprises a β-lactam moiety, thereby treating the bacterial infection.

In another aspect, the invention provides a method of killing or inhibiting the growth of a bacteria, said method comprising: contacting said bacteria with an effective amount of a compound of the invention or a combination of the invention, or a pharmaceutically acceptable salt thereof, thereby killing or inhibiting the growth of the bacteria.

In another aspect, the invention provides a method of inhibiting a β-lactamase, comprising contacting the β-lactamase with an effective amount of a compound of the invention, thereby inhibiting the β-lactamase.

In another aspect, the invention provides a method of treating a bacterial infection comprising: administering to an animal suffering from said infection an effective amount of a compound of the invention, thereby treating the bacterial infection.

In another aspect, the invention provides a method of inhibiting the editing domain of a t-RNA synthetase, comprising: contacting the synthetase with an effective amount of a compound of the invention, thereby inhibiting the synthetase.

In another aspect, the invention provides a use of a compound of the invention or a combination of the invention, in the manufacture of a medicament for the treatment and/or prophylaxis of bacterial infection.

### BRIEF DESCRIPTION OF THE DRAWINGS

FIG. 1 displays exemplary compounds of the invention.

FIG. 2 displays biological data for exemplary compounds of the invention.

FIG. 3 displays biological data for exemplary compounds of the invention. ‘ND’ stands for a value that was not determined.

### DETAILED DESCRIPTION OF THE INVENTION

### I. Definitions and Abbreviations

The abbreviations used herein generally have their conventional meaning within the chemical and biological arts.

“Compound of the invention,” as used herein refers to the compounds discussed herein, salts (e.g. pharmaceutically acceptable salts), prodrugs, solvates and hydrates of these compounds.

“Combination of the invention,” as used herein refers to the compounds and antibiotics discussed herein as well as acids, bases, salt forms (such as pharmaceutically acceptable salts), prodrugs, solvates and hydrates of these compounds and antibiotics.

“Boron containing compounds”, as used herein, refers to the compounds of the invention that contain boron as part of their chemical formula.

MIC, or minimum inhibitory concentration, is the point where the compound stops more than 50% of cell growth, preferably 60% of cell growth, preferably 70% of cell growth, preferably 80% of cell growth, preferably 90% of cell growth, relative to an untreated control.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “poly” as used herein means at least 2. For example, a polyvalent metal ion is a metal ion having a valency of at least 2.

“Moiety” refers to a radical of a molecule that is attached to the remainder of the molecule.

The symbol [symbol] , whether utilized as a bond or displayed perpendicular to a bond, indicates the point at which the displayed moiety is attached to the remainder of the molecule.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). In some embodiments, the term “alkyl” means a straight or branched chain, or combinations thereof, which may be fully saturated, mono- or polyunsaturated and can include di- and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom. In some embodiments, the term “heteroalkyl,” by itself or in combination with another term, means a stable straight or branched chain, or combinations thereof, consisting of the stated number of carbon atoms and at least one heteroatom. In an exemplary embodiment, the heteroatoms can be selected from the group consisting of B, O, N and S, and wherein the nitrogen and sulfur atoms may optionally be oxidized and the nitrogen heteroatom may optionally be quaternized. The heteroatom(s) B, O, N and S may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms. In an exemplary embodiment, the heteroatom is selected from B, N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, 6-quinolyl, dioxaborolane, dioxaborinane and dioxaborepane. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —R′, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR′″″—C(NR′R″R″′)═NR″″, —NR″″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to (2m′+1), where m′ is the total number of carbon atoms in such radical. R′, R″, R″′, R″″ and R′″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″, R″″ and R′″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR″″′—C(NR′R″R′″)═NR″″, —NR″″—C(NR′R″)═NR′″, —S(O)R′, —S(O)₂NR′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R″′, R″″ and R″″′ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″, R″″ and R′″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

“Ring” as used herein, means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 to 7 atoms in the encircling arrangement. Unless otherwise specified, the ring optionally includes a heteroatom. Thus, the term “5- to 7-membered ring” includes, for example phenyl, pyridinyl and piperidinyl. The term “5- to 7-membered heterocycloalkyl ring”, on the other hand, would include pyridinyl and piperidinyl, but not phenyl. The term “ring” further includes a ring system comprising more than one “ring”, wherein each “ring” is independently defined as above.

As used herein, the term “heteroatom” includes atoms other than carbon (C) and hydrogen (H). Examples include oxygen (O), nitrogen (N) sulfur (S), silicon (Si), germanium (Ge), aluminum (Al) and boron (B).

The term “leaving group” means a functional group or atom which can be displaced by another functional group or atom in a substitution reaction, such as a nucleophilic substitution reaction. By way of example, representative leaving groups include triflate, chloro, bromo and iodo groups; sulfonic ester groups, such as mesylate, tosylate, brosylate, nosylate and the like; and acyloxy groups, such as acetoxy, trifluoroacetoxy and the like.

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl groups.

By “effective” amount of a drug, formulation, or permeant is meant a sufficient amount of an active agent to provide the desired local or systemic effect. A “Topically effective,” “Cosmetically effective,” “pharmaceutically effective,” or “therapeutically effective” amount refers to the amount of drug needed to effect the desired therapeutic result.

“Topically effective” refers to a material that, when applied to the skin, nail, hair, claw or hoof produces a desired pharmacological result either locally at the place of application or systemically as a result of transdermal passage of an active ingredient in the material.

“Cosmetically effective” refers to a material that, when applied to the skin, nail, hair, claw or hoof, produces a desired cosmetic result locally at the place of application of an active ingredient in the material.

The term “pharmaceutically acceptable salt” is meant to include a salt of a compound of the invention which are prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the present invention provides compounds which are in a prodrug form. Prodrugs of the compounds described herein readily undergo chemical changes under physiological conditions to provide the compounds of the invention. Additionally, prodrugs can be converted to the compounds of the invention by chemical or biochemical methods in an ex vivo environment.

Certain compounds of the invention can exist in unsolvated forms as well as solvated forms, including hydrated forms. In general, the solvated forms are equivalent to unsolvated forms and are encompassed within the scope of the present invention. Certain compounds of the invention may exist in multiple crystalline or amorphous forms.

Certain compounds of the invention possess asymmetric carbon atoms (optical centers) or double bonds; the racemates, diastereomers, geometric isomers and individual isomers are encompassed within the scope of the present invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the present invention.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

“Pharmaceutically acceptable topical carrier” and equivalent terms refer to pharmaceutically acceptable carriers, as described herein above, suitable for topical application. An inactive liquid or cream vehicle capable of suspending or dissolving the active agent(s), and having the properties of being nontoxic and non-inflammatory when applied to the skin, nail, hair, claw or hoof is an example of a pharmaceutically-acceptable topical carrier. This term is specifically intended to encompass carrier materials approved for use in topical cosmetics as well.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The terms “enhancement,” “penetration enhancement” or “permeation enhancement” relate to an increase in the permeability of the skin, nail, hair, claw or hoof to a drug, so as to increase the rate at which the drug permeates through the skin, nail, hair, claw or hoof. The enhanced permeation effected through the use of such enhancers can be observed, for example, by measuring the rate of diffusion of the drug through animal skin, nail, hair, claw or hoof using a diffusion cell apparatus. A diffusion cell is described by Merritt et al. Diffusion Apparatus for Skin Penetration, J of Controlled Release, 1 (1984) pp. 161-162. The term “permeation enhancer” or “penetration enhancer” intends an agent or a mixture of agents, which, alone or in combination, act to increase the permeability of the skin, nail, hair or hoof to a drug.

The term “excipients” is conventionally known to mean carriers, diluents and/or vehicles used in formulating drug compositions effective for the desired use.

The term “topical administration” refers to the application of a pharmaceutical agent to the external surface of the skin, nail, hair, claw or hoof, such that the agent crosses the external surface of the skin, nail, hair, claw or hoof and enters the underlying tissues. Topical administration includes application of the composition to intact skin, nail, hair, claw or hoof, or to a broken, raw or open wound of skin, nail, hair, claw or hoof. Topical administration of a pharmaceutical agent can result in a limited distribution of the agent to the skin and surrounding tissues or, when the agent is removed from the treatment area by the bloodstream, can result in systemic distribution of the agent.

The term “transdermal delivery” refers to the diffusion of an agent across the barrier of the skin, nail, hair, claw or hoof resulting from topical administration or other application of a composition. The stratum corneum acts as a barrier and few pharmaceutical agents are able to penetrate intact skin. In contrast, the epidermis and dermis are permeable to many solutes and absorption of drugs therefore occurs more readily through skin, nail, hair, claw or hoof that is abraded or otherwise stripped of the stratum corneum to expose the epidermis. Transdermal delivery includes injection or other delivery through any portion of the skin, nail, hair, claw or hoof or mucous membrane and absorption or permeation through the remaining portion. Absorption through intact skin, nail, hair, claw or hoof can be enhanced by placing the active agent in an appropriate pharmaceutically acceptable vehicle before application to the skin, nail, hair, claw or hoof. Passive topical administration may consist of applying the active agent directly to the treatment site in combination with emollients or penetration enhancers. As used herein, transdermal delivery is intended to include delivery by permeation through or past the integument, i.e. skin, nail, hair, claw or hoof.

The terms “effective amount” or a “therapeutically effective amount” of a drug or pharmacologically active agent refers to a nontoxic but sufficient amount of the drug or agent to provide the desired effect. In the oral dosage forms of the present disclosure, an “effective amount” of one active of the combination is the amount of that active that is effective to provide the desired effect when used in combination with the other active of the combination. The amount that is “effective” will vary from subject to subject, depending on the age and general condition of the individual, the particular active agent or agents, and the appropriate “effective” amount in any individual case may be determined by one of ordinary skill in the art using routine experimentation.

The phrases “active ingredient”, “therapeutic agent”, “active”, or “active agent” mean a chemical entity which can be effective in treating a targeted disorder, disease or condition.

The phrase “pharmaceutically acceptable” means moieties or compounds that are, within the scope of medical judgment, suitable for use in humans without causing undesirable biological effects such as undue toxicity, irritation, allergic response, and the like, for example.

The phrase “oral dosage form” means any pharmaceutical composition administered to a subject via the oral cavity. Exemplary oral dosage forms include tablets, capsules, films, powders, sachets, granules, solutions, solids, suspensions or as more than one distinct unit (e.g., granules, tablets, and/or capsules containing different actives) packaged together for co-administration, and other formulations known in the art. An oral dosage form can be one, two, three, four, five or six units. When the oral dosage form has multiple units, all of the units are contained within a single package, (e.g. a bottle or other form of packaging such as a blister pack). When the oral dosage form is a single unit, it may or may not be in a single package. In a preferred embodiment, the oral dosage form is one, two or three units. In a particularly preferred embodiment, the oral dosage form is one unit.

The phrase “unit”, as used herein, refers to the number of discrete objects to be administered which comprise the dosage form. In some embodiments, the dosage form includes a compound of the invention in one capsule. This is a single unit. In some embodiments, the dosage form includes a compound of the invention as part of a therapeutically effective dosage of a cream or ointment. This is also a single unit. In some embodiments, the dosage form includes a compound of the invention and another active ingredient contained within one capsule, or as part of a therapeutically effective dosage of a cream or ointment. This is a single unit, whether or not the interior of the capsule includes multiple discrete granules of the active ingredient. In some embodiments, the dosage form includes a compound of the invention in one capsule, and the active ingredient in a second capsule. This is a two unit dosage form, such as two capsules or tablets, and so such units are contained in a single package. Thus the term ‘unit’ refers to the object which is administered to the animal, not to the interior components of the object.

The term, “prodrug”, as defined herein, is a derivative of a parent drug molecule that exerts its pharmacological effect only after chemical and/or enzymatic conversion to its active form in vivo. Prodrugs include those designed to circumvent problems associated with delivery of the parent drug. This may be due to poor physicochemical properties, such as poor chemical stability or low aqueous solubility, and may also be due to poor pharmacokinetic properties, such as poor bioavailability or poor half-life. Thus, certain advantages of prodrugs may include improved chemical stability, absorption, and/or PK properties of the parent carboxylic acids. Prodrugs may also be used to make drugs more “patient friendly,” by minimizing the frequency (e.g., once daily) or route of dosing (e.g., oral), or to improve the taste or odor if given orally, or to minimize pain if given parenterally.

In some embodiments, the prodrugs are chemically more stable than the active drug, thereby improving formulation and delivery of the parent drug, compared to the drug alone.

Prodrugs for carboxylic acid analogs of the invention may include a variety of esters. In an exemplary embodiment, the pharmaceutical compositions of the invention include a carboxylic acid ester. In an exemplary embodiment, the prodrug is suitable for treatment/prevention of those diseases and conditions that require the drug molecule to cross the blood brain barrier. In an exemplary embodiment, the prodrug enters the brain, where it is converted into the active form of the drug molecule. In one embodiment, a prodrug is used to enable an active drug molecule to reach the inside of the eye after topical application of the prodrug to the eye. Additionally, a prodrug can be converted to its parent compound by chemical or biochemical methods in an ex vivo environment. For example, a prodrug can be slowly converted to its parent compound when placed in a transdermal patch reservoir with a suitable enzyme or chemical reagent.

“Antibiotic”, as used herein, is a compound which can kill or inhibit the growth of bacteria. The term antibiotic is broad enough to encompass acids, bases, salt forms (such as pharmaceutically acceptable salts), prodrugs, solvates and hydrates of the antibiotic compound.

The term “microbial infection” or “infection by a microorganism” refers to any infection of a host by an infectious agent including, but not limited to, viruses, bacteria, mycobacteria, fungus and parasites (see, e.g., Harrison's Principles of Internal Medicine, pp. 93-98 (Wilson et al., eds., 12th ed. 1991); Williams et al., J. of Medicinal Chem. 42:1481-1485 (1999), herein each incorporated by reference in their entirety).

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Inhibiting” and “blocking,” are used interchangeably herein to refer to the partial or full blockade of an enzyme, such as a β-lactamase or a LeuRS.

The term “leaving group” means a functional group or atom which can be displaced by another functional group or atom in a substitution reaction, such as a nucleophilic substitution reaction. By way of example, representative leaving groups include triflate, chloro, bromo and iodo groups; sulfonic ester groups, such as mesylate, tosylate, brosylate, nosylate and the like; and acyloxy groups, such as acetoxy, trifluoroacetoxy and the like.

The term “amino-protecting group” means a protecting group suitable for preventing undesired reactions at an amino nitrogen. Representative amino-protecting groups include, but are not limited to, formyl; acyl groups, for example alkanoyl groups, such as acetyl, trichloroacetyl or trifluoroacetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBS); and the like.

The term “hydroxy-protecting group” means a protecting group suitable for preventing undesired reactions at a hydroxy group. Representative hydroxy-protecting groups include, but are not limited to, alkyl groups, such as methyl, ethyl, and tert-butyl; acyl groups, for example alkanoyl groups, such as acetyl; arylmethyl groups, such as benzyl (Bn), p-methoxybenzyl (PMB), 9-fluorenylmethyl (Fm), and diphenylmethyl (benzhydryl, DPM); silyl groups, such as trimethylsilyl (TMS) and tert-butyldimethylsilyl (TBS); and the like.

Boron is able to form dative bonds with oxygen, sulfur or nitrogen under some circumstances in this invention. Dative bonds are usually weaker than covalent bonds. In situations where a boron is covalently bonded to at least one oxygen, sulfur or nitrogen, and is at the same time datively bonded to an oxygen, sulfur or nitrogen, respectively, the dative bond and covalent bond between the boron and the two identical heteroatoms can interconvert or be in the form of a resonance hybrid. There is potential uncertainty surrounding the exact nature and extent of electron sharing in these situations. The structures supplied are not intended to include any and all possible bonding scenarios between boron and the atom to which it is bound. Non limiting examples of these bonds are as follows:

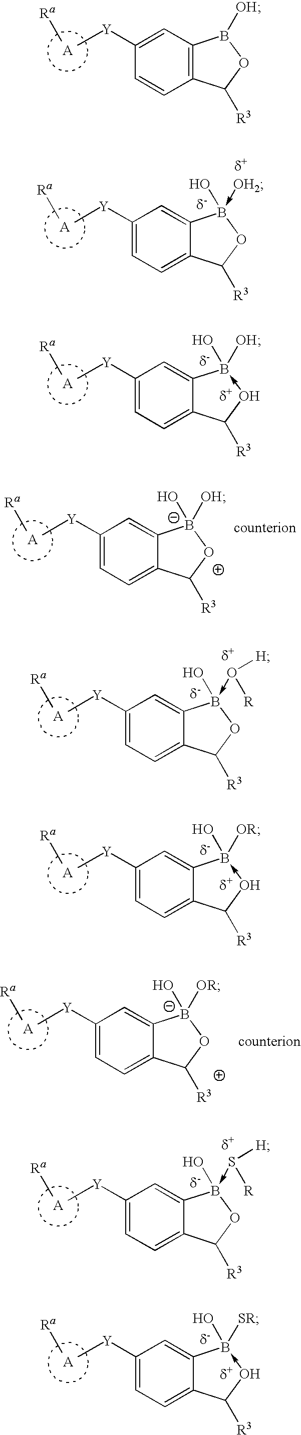

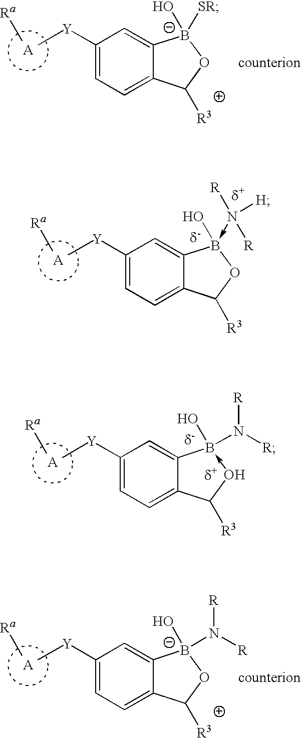

“Salt counterion”, as used herein, refers to positively charged ions that associate with a compound of the invention when the boron is fully negatively or partially negatively charged. Examples of salt counterions include H⁺, H₃O⁺, ammonium, potassium, calcium, magnesium and sodium.

The compounds comprising a boron bonded to a carbon and three heteroatoms (such as three oxygens described in this section) can optionally contain a fully negatively charged boron or partially negatively charged boron, due to the nature of the dative bond between the boron and one of the oxygens. Due to the negative charge, a positively charged counterion may associate with this compound, thus forming a salt. Examples of positively charged counterions include H⁺, H₃O⁺, calcium, sodium, ammonium, potassium. The salts of these compounds are implicitly contained in descriptions of these compounds.

The present invention also encompasses compounds that are poly- or multi-valent species, including, for example, species such as dimers, trimers, tetramers and higher homologs of the compounds of use in the invention or reactive analogues thereof. For example, dimers of oxaboroles can form under the following conditions:

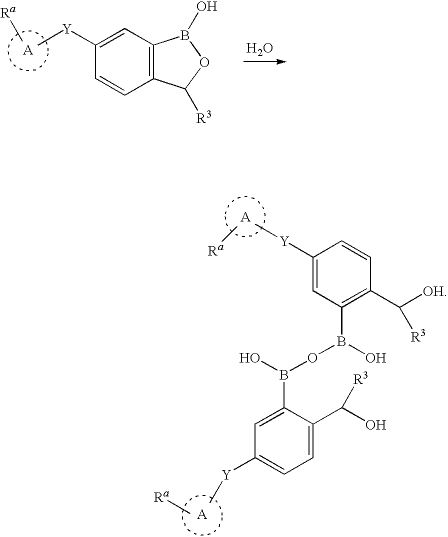

The present invention also encompasses compounds that are anhydrides of the cyclic boronic esters are synthesized by subjecting these compounds to dehydrating conditions. Examples of these anhydrides are provided below:

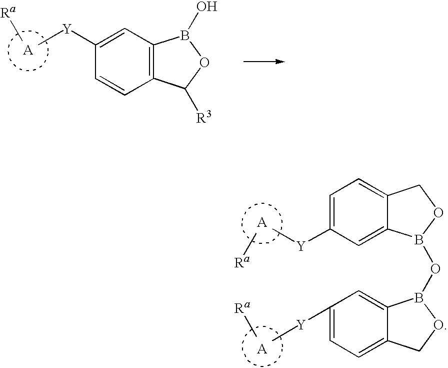

Trimers of the compounds of the invention are also produced. For example, trimers of acyclic boronic esters can be formed as follows:

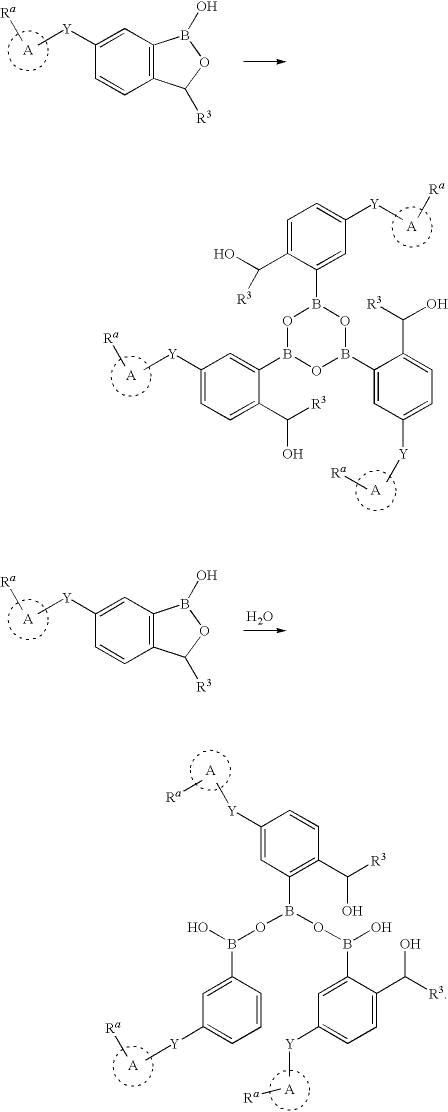

Polymers of the compounds of the invention are also produced through the removal of certain protecting groups in strong acid. For example, trimers of acyclic boronic esters can be formed as follows:

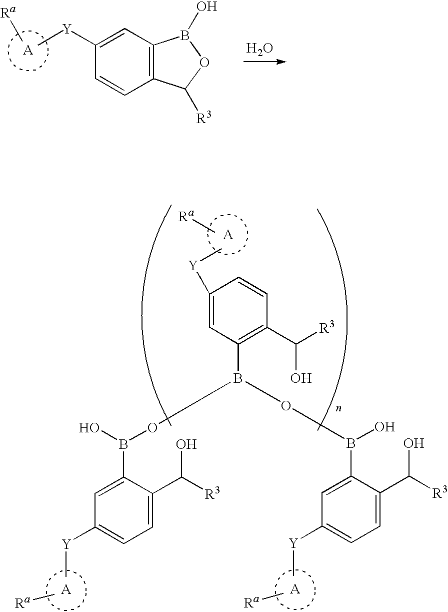

Also of use in the present invention are compounds that are poly- or multi-valent species, including, for example, species such as dimers, trimers, tetramers and higher homologs of the compounds of use in the invention or reactive analogues thereof. The poly- and multi-valent species can be assembled from a single species or more than one species of the invention. For example, a dimeric construct can be “homo-dimeric” or “heterodimeric.” Moreover, poly- and multi-valent constructs in which a compound of the invention or a reactive analogue thereof, is attached to an oligomeric or polymeric framework (e.g., polylysine, dextran, hydroxyethyl starch and the like) are within the scope of the present invention. The framework is preferably polyfunctional (i.e. having an array of reactive sites for attaching compounds of use in the invention). Moreover, the framework can be derivatized with a single species of the invention or more than one species of the invention.

Moreover, the present invention includes the use of compounds within the motif set forth in the formulae contained herein, which are functionalized to afford compounds having water-solubility that is enhanced relative to analogous compounds that are not similarly functionalized. Thus, any of the substituents set forth herein can be replaced with analogous radicals that have enhanced water solubility. For example, it is within the scope of the invention to replace a hydroxyl group with a diol, or an amine with a quaternary amine, hydroxy amine or similar more water-soluble moiety. In a preferred embodiment, additional water solubility is imparted by substitution at a site not essential for the activity towards the editing domain of the compounds set forth herein with a moiety that enhances the water solubility of the parent compounds. Methods of enhancing the water-solubility of organic compounds are known in the art. Such methods include, but are not limited to, functionalizing an organic nucleus with a permanently charged moiety, e.g., quaternary ammonium, or a group that is charged at a physiologically relevant pH, e.g. carboxylic acid, amine. Other methods include, appending to the organic nucleus hydroxyl- or amine-containing groups, e.g. alcohols, polyols, polyethers, and the like. Representative examples include, but are not limited to, polylysine, polyethyleneimine, poly(ethyleneglycol) and poly(propyleneglycol). Suitable functionalization chemistries and strategies for these compounds are known in the art. See, for example, Dunn, R. L., et al., Eds. POLYMERIC DRUGS AND DRUG DELIVERY SYSTEMS, ACS Symposium Series Vol. 469, American Chemical Society, Washington, D.C. 1991.

### II. Introduction

The present invention provides novel boron compounds and methods for the preparation of these molecules. The invention further provides methods of treating bacterial infections, killing or inhibiting the growth of bacteria, and/or inhibiting β-lactamase in part or wholly through the use of the compounds described herein. The invention further provides methods of treating anti-inflammatory conditions and inhibiting biomolecules that are implicated with anti-inflammatory conditions in part or wholly through the use of the compounds described herein. In another aspect, the invention is a combination of a compound of the invention and an antibiotic. In another aspect, the invention is a pharmaceutical formulation comprising a pharmaceutically acceptable excipient and a compound of the invention. In another aspect, the invention is a pharmaceutical formulation comprising a compound of the invention, an antibiotic, and a pharmaceutically acceptable excipient.

### III. a.) Compounds

In one aspect the invention provides a compound of the invention. In an exemplary embodiment, the invention provides a compound described herein, or a salt thereof. In an exemplary embodiment, the salt of a compound described herein is a pharmaceutically acceptable salt. In an exemplary embodiment, the invention provides a compound described herein, or a pharmaceutically acceptable salt thereof. In an exemplary embodiment, the invention provides a compound described in a formula provided herein. In an exemplary embodiment, the invention provides a compound described herein. In one aspect, the invention provides a compound having a structure according to the formula:

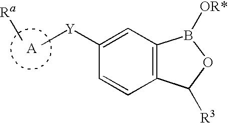

wherein R* is a member selected from H, a negative charge and a positively charged counterion. A is a member selected from substituted or unsubstituted cycloalkyl, substituted or unsubstituted heterocycloalkyl, substituted or unsubstituted aryl and substituted or unsubstituted heteroaryl. Y is a member selected from O, S, unsubstituted C₁-C₄ alkyl and —S(O)₂NH—, wherein the sulfur in the —S(O)₂NH— is covalently attached to the A ring. R³ is a member selected from H, cyano and substituted or unsubstituted alkyl. R^{a} is a member selected from H, OR , NR¹⁰R¹¹, SR¹⁰, —S(O)R¹⁰, —S(O)₂R¹⁰, —S(O)₂NR¹⁰R¹¹, —C(O)R¹⁰, —C(O)OR¹⁰, —C(O)NR¹⁰R¹¹, nitro, cyano, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, each R¹⁰ and each R¹¹ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. There is a proviso that R¹⁰ and R¹¹, together with the nitrogen to which they are attached, are optionally combined to form a 5- to 7-membered substituted or unsubstituted heterocycloalkyl ring. In an exemplary embodiment, there is a proviso that when Y is —S(O)₂NH—, R³ is H, and R^{a} is not H or unsubstituted alkyl or halosubstituted alkyl. In an exemplary embodiment, there is a proviso that when Y is O, R³ is a member selected from cyano and substituted alkyl.

In an exemplary embodiment, the invention provides a compound having a structure according to the formula:

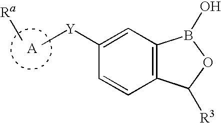

wherein A is a member selected from cycloalkyl, heterocycloalkyl, aryl and heteroaryl; Y is a member selected from O and —S(O)₂NH— wherein the sulfur in —S(O)₂NH— is covalently attached to A; R³ is a member selected from H, cyano and substituted alkyl; R^{a} is a member selected from H, —OR¹⁰, —NR¹⁰R¹¹, —SR₁₀, —S(O)R¹⁰, —S(O)₂R¹⁰, —S(O)₂NR¹⁰R¹¹, —C(O)¹⁰, —C(O)NR¹⁰R¹¹, nitro, cyano, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl wherein each R¹⁰ and each R¹¹ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, with the proviso that R¹⁰ and R¹¹, together with the nitrogen to which they are attached, are optionally combined to form a 5- to 7-membered substituted or unsubstituted heterocycloalkyl ring; with the proviso that when Y is O, R³ is a member selected from cyano and substituted alkyl; with the proviso that when Y is —S(O)₂NH—, R³ is H, and R^{a} is not H or unsubstituted alkyl or halosubstituted alkyl, and salts thereof.

In an exemplary embodiment, the compound has a structure according to the following formula:

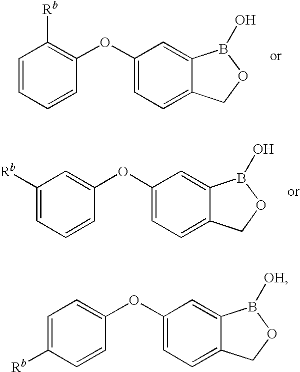

wherein R^{b} is halogen, or salts thereof. In an exemplary embodiment, R^{b} is F. In an exemplary embodiment, R^{b} is Cl. In an exemplary embodiment, R^{b} is Br.

In an exemplary embodiment, the compound has a structure according to the following formula:

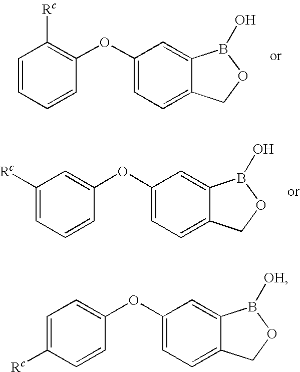

wherein R^{c} is hydroxyalkyl, or salts thereof. In an exemplary embodiment, R^{c} is —(CH₂)ₘ₁OH, wherein m1 is 1 or 2 or 3 or 4 or 5 or 6. In an exemplary embodiment, m1 is 1 or 2 or 3. In an exemplary embodiment, R^{c} is —CH₂OH.

In an exemplary embodiment, the compound has a structure according to the following formula:

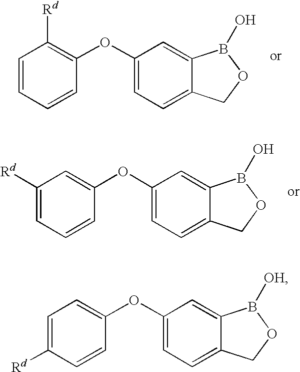

wherein R^{d} is aminoalkyl, or salts thereof. In an exemplary embodiment, R^{d} is —(CR¹²R¹³)—NR¹⁴R¹⁵ in which n is a member selected from 1 to 10; each R¹² and each R¹³ is a member independently selected from H, OR¹⁶, NR¹⁶R¹⁷, SR¹⁶, —S(O)R¹⁶, —S(O)₂R¹⁶, —S(O)₂NR¹⁶R¹⁷, —C(O)R¹⁷, —C(O)OR¹⁷, —C(O)NR¹⁶R¹⁷, nitro, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R¹⁴ and R¹⁵ are members independently selected from H, OR¹⁸, NR¹⁸R¹⁹, SR¹⁸, —S(O)R¹⁸, —S(O)₂R¹⁸, —S(O)₂NR¹⁸R¹⁹, —C(O)R¹⁹, —C(O)OR¹⁹, —C(O)NR¹⁸R¹⁹, nitro, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. Each R¹⁶, each R¹⁷, each R¹⁸ and each R¹⁹ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In an exemplary embodiment, R^{d} is —(CR¹²R¹³)—NH₂, wherein n is 1 or 2 or 3 or 4 or 5 or 6, wherein R¹² and R¹³ are as described herein. In an exemplary embodiment, R^{d} is —(CH₂)ₙNR¹⁴R¹⁵. In an exemplary embodiment, R^{d} is —CH₂NR¹⁴R¹⁵. In an exemplary embodiment, R^{d} is —CH₂NH₂.

In an exemplary embodiment, the compound has a structure according to the following formula:

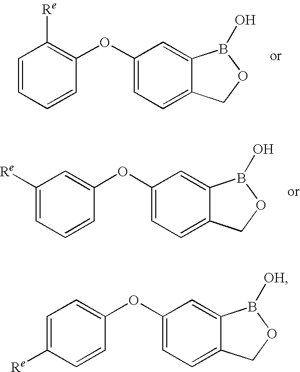

wherein R^{e} is —C(O)OR¹⁰, or salts thereof, wherein R¹⁰ is H or substituted or unsubstituted alkyl. In an exemplary embodiment, R^{e} is —C(O)OR¹⁰, wherein R¹⁰ is unsubstituted C₁ or C₂ or C₃ or C₄ or C₅ or C₆ alkyl. In an exemplary embodiment, wherein R¹⁰ is unsubstituted C₁ or C₂ or C₃ alkyl. In an exemplary embodiment, R^{e} is —COOH or —COOCH₃ or —COOCH₂CH₃ or —COOC(CH₃)₃.

In an exemplary embodiment, the compound has a structure according to the following formula:

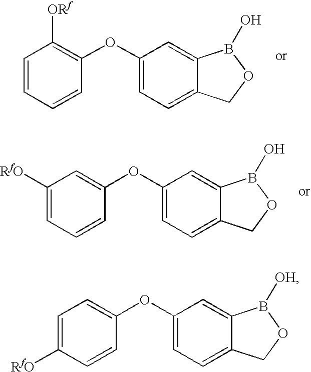

wherein R^{f} is H or substituted or unsubstituted alkyl, or salts thereof. In an exemplary embodiment, R^{f} is H. In an exemplary embodiment, R^{f} is unsubstituted C₁ or C₂ or C₃ or C₄ or C₅ or C₆ alkyl. In an exemplary embodiment, R^{f} is methyl. In an exemplary embodiment, R^{f} is phenylsubstituted alkyl. In an exemplary embodiment, R^{f} is phenyl substituted C₁ or C₂ or C₃ or C₄ or C₅ or C₆ alkyl. In an exemplary embodiment, R^{f} is phenylmethyl.

In an exemplary embodiment, the compound has a structure according to the following formula:

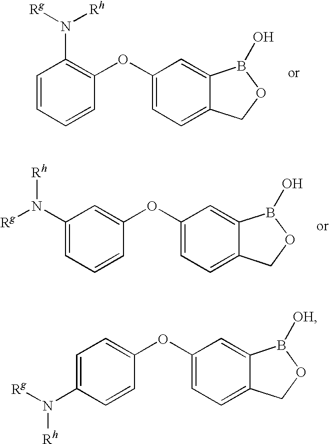

wherein R^{g} and R^{h} is independently selected from H or substituted or unsubstituted alkyl, or salts thereof. In an exemplary embodiment, R^{g} is H. In an exemplary embodiment, R^{h} is H. In an exemplary embodiment, R^{g} is H and R^{h} is H. In an exemplary embodiment, R^{g} is unsubstituted alkyl, and R^{h} is as described herein.

In an exemplary embodiment, the compound has a structure according to the following formula:

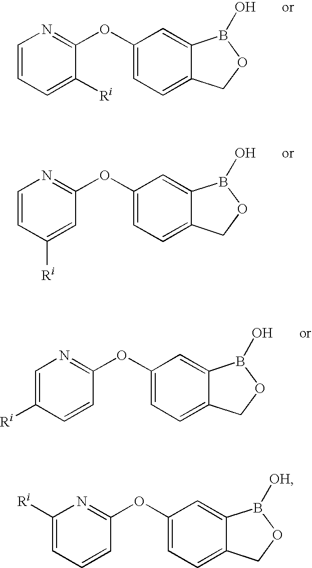

wherein Rⁱ is cyano, or salts thereof.

In an exemplary embodiment, the compound has a structure according to the following formula:

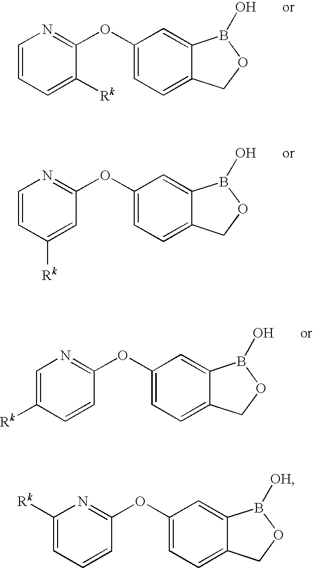

wherein R^{k} is aminoalkyl, or a salts thereof. In an exemplary embodiment, R^{k} is —(CH₂)ₘ₁NH₂, wherein m1 is 1 or 2 or 3 or 4 or 5 or 6. In an exemplary embodiment, m1 is 1 or 2 or 3. In an exemplary embodiment, R^{k} is —CH₂NH₂.

In an exemplary embodiment, the compound has a structure according to the following formula:

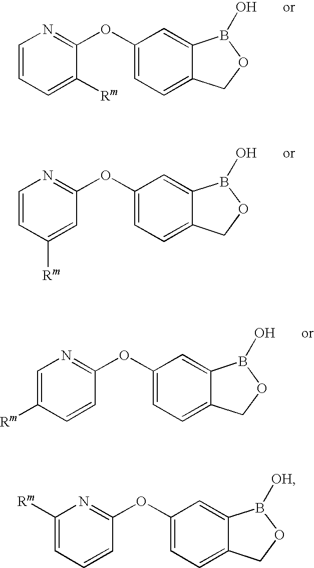

wherein R^{m} is —C(O)OR¹⁰, or salts thereof, wherein R¹⁰ is H or substituted or unsubstituted alkyl. In an exemplary embodiment, R^{m} is —C(O)OR¹⁰, wherein R¹⁰ is unsubstituted C₁ or C₂ or C₃ or C₄ or C₅ or C₆ alkyl. In an exemplary embodiment, wherein R¹⁰ is unsubstituted C₁ or C₂ or C₃ alkyl. In an exemplary embodiment, R^{m} is —COOH or —COOCH₃ or —COOCH₂CH₃ or —COOC(CH₃)₃.

In an exemplary embodiment, the compound has a structure according to the following formula:

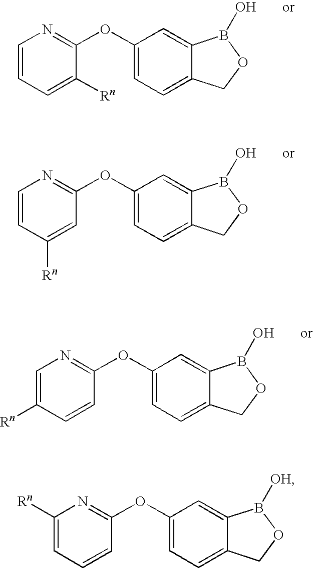

wherein Rⁿ is —C(O)NR¹¹R¹², or salts thereof, wherein each R¹¹ or R¹² is a member selected from H or substituted or unsubstituted alkyl. In an exemplary embodiment, R¹¹ is H. In an exemplary embodiment, Rₙ is —C(O)NH₂.

In an exemplary embodiment, the compound has a structure according to the following formula:

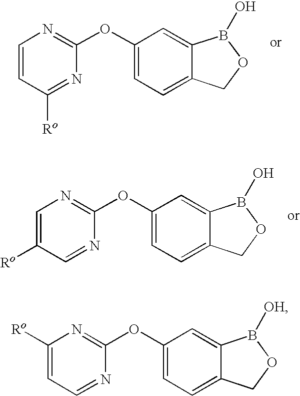

wherein R^{o} is H or —C(O)OR¹⁰, or salts thereof, wherein R¹⁰ is H or substituted or unsubstituted alkyl. In an exemplary embodiment, R^{o} is —C(O)OR¹⁰, wherein R¹⁰ is unsubstituted C₁ or C₂ or C₃ or C₄ or C₅ or C₆ alkyl. In an exemplary embodiment, wherein R¹⁰ is unsubstituted C₁ or C₂ or C₃ alkyl. In an exemplary embodiment, R^{o} is H or —COOH or —COOCH₃ or —COOCH₂CH₃ or —COOC(CH₃)₃.

In an exemplary embodiment, the compound has a structure according to the following formula:

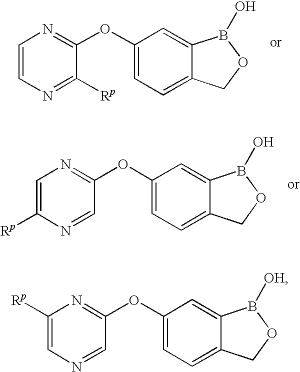

wherein R^{p} is H or —C(O)OR¹⁰, or salts thereof, wherein R¹⁰ is H or substituted or unsubstituted alkyl. In an exemplary embodiment, R^{p} is —C(O)0R¹⁰, wherein R¹⁰ is unsubstituted C₁ or C₂ or C₃ or C₄ or C₅ or C₆ alkyl. In an exemplary embodiment, wherein R¹⁰ is unsubstituted C₁ or C₂ or C₃ alkyl. In an exemplary embodiment, R^{p} is H or —COOH or —COOCH₃ or —COOCH₂CH₃ or —COOC(CH₃)₃.

In an exemplary embodiment, the compound has a structure according to the following formula:

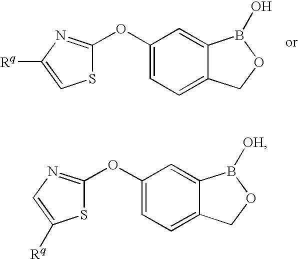

wherein R^{q} is H or —C(O)OR¹⁰, or salts thereof, wherein R¹⁰ is H or substituted or unsubstituted alkyl. In an exemplary embodiment, R^{q} is —C(O)OR¹⁰, wherein R¹⁰ is unsubstituted C₁ or C₂ or C₃ or C₄ or C₅ or C₆ alkyl. In an exemplary embodiment, wherein R¹⁰ is unsubstituted C₁ or C₂ or C₃ alkyl. In an exemplary embodiment, R^{q} is H or —COOH or —COOCH₃ or —COOCH₂CH₃ or —COOC(CH₃)₃.

In an exemplary embodiment, the compound is

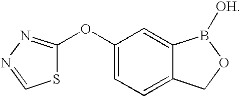

In an exemplary embodiment, the compound is

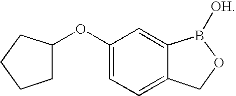

In an exemplary embodiment, the compound is

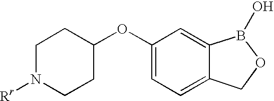

wherein R^{r} is H or —C(O)OR¹⁰, or salts thereof, wherein R¹⁰ is H or substituted or unsubstituted alkyl. In an exemplary embodiment, R^{r} is —C(O)OR¹⁰, wherein R¹⁰ is unsubstituted C₁ or C₂ or C₃ or C₄ or C₅ or C₆ alkyl. In an exemplary embodiment, wherein R¹⁰ is unsubstituted C₁ or C₂ or C₃ alkyl. In an exemplary embodiment, R^{r} is H or —COOH or —COOCH₃ or —COOCH₂CH₃ or —COOC(CH₃)₃.

In an exemplary embodiment, the compound has a structure according to the formula:

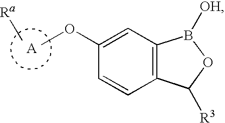

wherein R^{a} and A are as described herein, and R³ is a member selected from cyano and substituted or unsubstituted alkyl.

In an exemplary embodiment, the compound has a structure according to the formula:

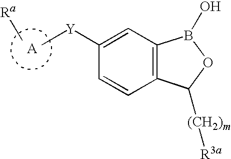

wherein m is an integer which is 1 or 2 or 3 or 4 or 5 or 6 and R^{3a} is a member selected from —C(O)OR²⁰ or —C(O)NR²⁰R²¹ or —OR₂₀ or nitro or —S(O)₂R²² or —S(O)₂OR²⁰ or —S(O)₂NR²⁰R²¹ or —P(O)(OR²⁰)(OR²⁰) wherein each R²⁰ is independently selected from H or unsubstituted alkyl, R²¹ is selected from H or —S(O)₂R²³; R²³ is unsubstituted alkyl. In an exemplary embodiment, m is 1 or 2 or 3. In an exemplary embodiment, m is 1.

In an exemplary embodiment, the compound has a structure according to the formula:

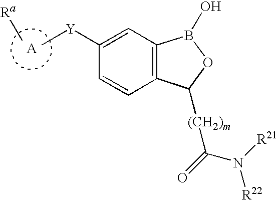

wherein m is an integer which is 1 or 2 or 3 or 4 or 5 or 6, R²¹ is selected from H or —S(O)₂R²³, R²² is unsubstituted alkyl and R²³ is unsubstituted alkyl.

In an exemplary embodiment, the compound has a structure according to the formula:

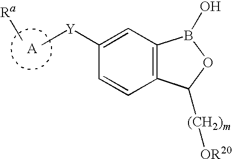

wherein m is an integer selected from 1 or 2 or 3 or 4 or 5 or 6 and R²⁰ is selected from H or unsubstituted alkyl. In an exemplary embodiment, m is 1 or 2 or 3. In an exemplary embodiment, m is 1.

In an exemplary embodiment, the compound has a structure according to the formula:

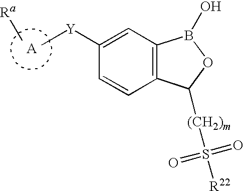

wherein R^{a}, A, Y, m and R²² are as described herein.

In an exemplary embodiment, the compound has a structure according to the formula:

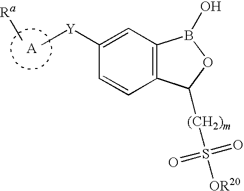

wherein R^{a}, A, Y, m and R²⁰ are as described herein.

In an exemplary embodiment, the compound has a structure according to the formula:

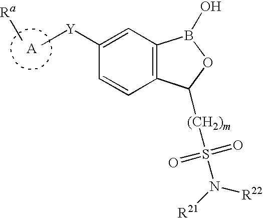

wherein R^{a}, A, Y, m, R²¹ and R²² are as described herein.

In an exemplary embodiment, the compound has a structure according to the formula:

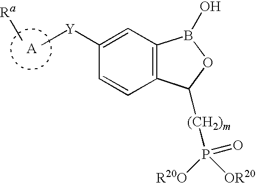

wherein R^{a}, A, Y, m and each R²⁰ are as described herein.

In an exemplary embodiment, the compound has a structure according to the formula:

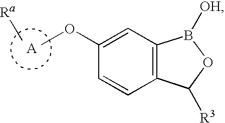

wherein A and R³ are as described herein, and R^{a} is a member selected from H, halogen, substituted or unsubstituted alkyl, OR¹⁰, NR¹⁰R¹¹, wherein R¹⁰ and each R¹¹ is a member independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, R^{a} is a member selected from H, F, Cl, —OR^{20a} and —C(O)OR^{20b}, wherein R^{20a} is alkyl, optionally substituted with a member selected from NH₂ and phenyl, and wherein R^{20b} is unsubstituted alkyl. In an exemplary embodiment, R^{a} is F or Cl. In an exemplary embodiment, R^{a} is OH. In an exemplary embodiment, R^{a} is NH₂. In an exemplary embodiment, R^{a} is —O(CH₂)—Ph or —(CH₂)ₙNH(CH₂)ₙ₁O(CH₂)ₙ₂CH₃ or —(CH₂)ₙX⁵ or —O(CH₂)ₙNH₂ or —NH(CH₂)ₙPh or —C(O)OR¹⁰, wherein n is 1 or 2 or 3 or 4 or 5 or 6, n1 is 1 or 2 or 3 or 4 or 5 or 6, n2 is 0 or 1 or 2 or 3 or 4 or 5 or 6, R^{10a} is unsubstituted alkyl, and X⁵ is unsubstituted morpholinyl or piperazinyl. In an exemplary embodiment, n is 1 or 2 or 3, or n1 is 1 or 2 or 3, or n2 is 0 or 1 or 2 or 3. In an exemplary embodiment, R^{a} is —C(O)OR^{10a} and R^{10a} is methyl or ethyl or propyl or isopropyl or tert-butyl. In an exemplary embodiment, R^{a} is —CH₂NH(CH₂)₂OCH₃ or —CH₂X⁵ or —O(CH₂)₃NH₂ or —OCH₂Ph or —NHCH₂Ph. In an exemplary embodiment, R^{a} is

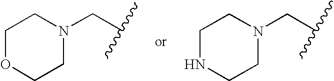

In an exemplary embodiment, the compound has a structure according to the formula:

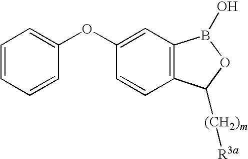

wherein m is an integer which is 1 or 2 or 3 or 4 or 5 or 6 and R^{3a} is a member selected from —C(O)OR²⁰ or —C(O)NR²⁰R²¹ or —OR²⁰ or nitro or —S(O)₂R²² or —S(O)₂OR²⁰ or —S(O)₂NR²⁰R²¹ or —P(O)(OR²⁰)(OR²⁰) wherein each R²⁰ is independently selected from H or unsubstituted alkyl, R²¹ is independently selected from H or —S(O)₂R²², and R²² is unsubstituted alkyl.

In an exemplary embodiment, the compound has a structure according to the formula:

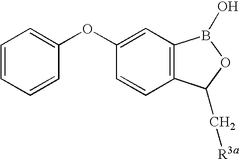

wherein R^{3a} is a member selected from —C(O)OR²⁰ or —C(O)_{NR}²⁰R²¹ or OR²⁰ or nitro or —S(O)₂R²² or —S(O)₂OR²⁰ or —S(O)₂NR²⁰R²¹ or —P(O)(OR)²⁰)(OR²⁰) wherein each R²⁰ is independently selected from H or unsubstituted alkyl, R²¹ is independently selected from H or —S(O)₂R²²; and R²² is unsubstituted alkyl.

In an exemplary embodiment, the compound has a structure according to the formula:

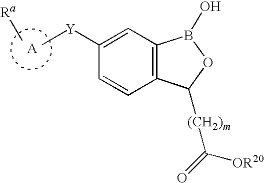

wherein m is an integer which is 1 or 2 or 3 or 4 or 5 or 6 and R²⁰ is H or unsubstituted alkyl. In an exemplary embodiment, m is 1 or 2 or 3. In an exemplary embodiment, R²⁰ is H. In an exemplary embodiment, R²⁰ is unsubstituted C₁ or C₂ or C₃ or C₄ or C₅ or C₆ alkyl. In an exemplary embodiment, R²⁰ is methyl or ethyl or t-butyl. In an exemplary embodiment, m is 1 or 2 or 3. In an exemplary embodiment, m is 1.

In an exemplary embodiment, the compound has a structure according to the formula:

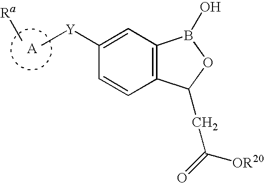

wherein m is an integer which is 1 or 2 or 3 or 4 or 5 or 6 and R²⁰ is H or unsubstituted alkyl. In an exemplary embodiment, m is 1 or 2 or 3. In an exemplary embodiment, R²⁰ is H. In an exemplary embodiment, R²⁰ is unsubstituted C₁ or C₂ or C₃.

In an exemplary embodiment, the compound has a structure according to the formula:

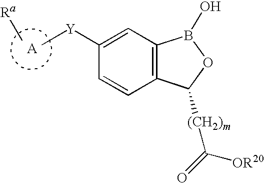

wherein m, R^{a}, A, Y and R²⁰ are as defined herein.

In an exemplary embodiment, the compound has a structure according to the formula:

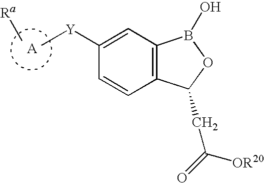

wherein R^{a}, A, Y and R²⁰ are as defined herein.

In an exemplary embodiment, the compound has a structure according to the formula:

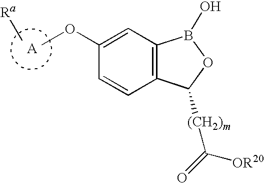

wherein m, R^{a}, A and R²⁰ are as defined herein. In an exemplary embodiment, R²⁰ is H. In an exemplary embodiment, R²⁰ is unsubstituted C₁ or C₂ or C₃ or C₄ or C₅ or C₆ alkyl. In an exemplary embodiment, R²⁰ is methyl or ethyl or t-butyl. In an exemplary embodiment, m is 1 or 2 or 3. In an exemplary embodiment, m is 1.

In an exemplary embodiment, the compound has a structure according to the formula:

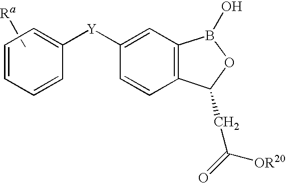

wherein R^{a}, Y and R²⁰ are as defined herein.

In an exemplary embodiment, the compound has a structure according to the formula:

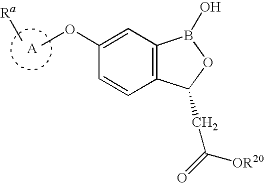

wherein R^{a}, A and R²⁰ are as defined herein.

In an exemplary embodiment, the compound has a structure according to the formula:

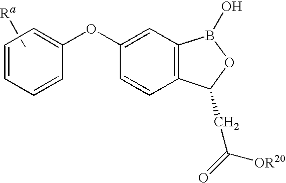

wherein R^{a} and R²⁰ are as defined herein.

In an exemplary embodiment, the compound has a structure according to the formula:

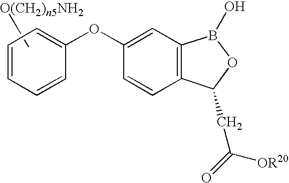

wherein R²⁰ is as defined herein, and n5 is an integer selected from 1 or 2 or 3 or 4 or 5 or 6. In an exemplary embodiment, the compound has a structure according to the formula:

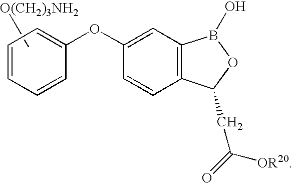

In an exemplary embodiment, the compound has a structure according to the formula:

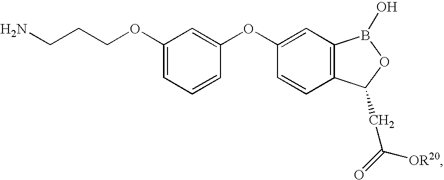

wherein R²⁰ is as defined herein. In an exemplary embodiment, R²⁰ is H.

In an exemplary embodiment, the compound is E38 or a salt thereof. In an exemplary embodiment, the compound is E38 or a pharmaceutically acceptable salt thereof. In an exemplary embodiment, the compound is E50 or a salt thereof. In an exemplary embodiment, the compound is E50 or a pharmaceutically acceptable salt thereof.

In an exemplary embodiment, the compound has a structure according to the formula:

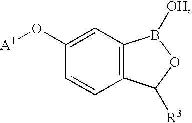

wherein A¹ is H or unsubstituted alkyl, and R³ is a member selected from cyano and substituted or unsubstituted alkyl. In an exemplary embodiment, A¹ is H. In an exemplary embodiment, A¹ is methyl. In an exemplary embodiment, A¹ is ethyl. In an exemplary embodiment, A¹ is C₃ alkyl. In an exemplary embodiment, A¹ is C₄ alkyl. In an exemplary embodiment, A¹ is C₅ alkyl. In an exemplary embodiment, A¹ is C₆ alkyl.

In an exemplary embodiment, the compound has a structure according to the formula:

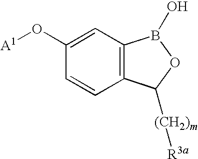

wherein A¹ is H or unsubstituted alkyl, m is an integer which is 1 or 2 or 3 or 4 or 5 or 6 and R^{3a} is a member selected from —C(O)OR²⁰ or —C(O)NR²⁰R²¹ or —OR₂₀ or nitro or —S(O)₂R²² or —S(O)₂₀ or —S(O)₂NR²⁰R²¹ or —P(O)(OR²⁰ )(OR²⁰) wherein each R²⁰ is independently selected from H or unsubstituted alkyl, R²¹ is selected from H or —S(O)₂R²³; R²³ is unsubstituted alkyl. In an exemplary embodiment, m is 1 or 2 or 3. In an exemplary embodiment, m is 1. In an exemplary embodiment, A¹ is H. In an exemplary embodiment, A¹ is methyl. In an exemplary embodiment, A¹ is ethyl. In an exemplary embodiment, A¹ is C₃ alkyl. In an exemplary embodiment, A¹ is C₄ alkyl. In an exemplary embodiment, A¹ is C₅ alkyl. In an exemplary embodiment, A¹ is C₆ alkyl.

In an exemplary embodiment, the compound has a structure according to the formula:

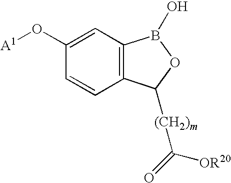

wherein A¹ is H or unsubstituted alkyl, m is an integer which is 1 or 2 or 3 or 4 or 5 or 6 and R²⁰ is H or unsubstituted alkyl. In an exemplary embodiment, m is 1 or 2 or 3. In an exemplary embodiment, m is 1. In an exemplary embodiment, R²⁰ is H. In an exemplary embodiment, R²⁰ is unsubstituted C₁ or C₂ or C₃ or C₄ or C₅ or C₆ alkyl. In an exemplary embodiment, R²⁰ is methyl or ethyl or t-butyl. In an exemplary embodiment, A¹ is H. In an exemplary embodiment, A¹ is methyl. In an exemplary embodiment, A¹ is ethyl. In an exemplary embodiment, A¹ is C₃ alkyl. In an exemplary embodiment, A¹ is C₄ alkyl. In an exemplary embodiment, A¹ is C₅ alkyl. In an exemplary embodiment, A¹ is C₆ alkyl.

In an exemplary embodiment, the compound has a structure according to the formula:

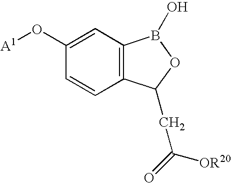

wherein A¹ and R²⁰ are as defined herein. In an exemplary embodiment, R²⁰ is H. In an exemplary embodiment, R²⁰ is methyl. In an exemplary embodiment, R²⁰ is ethyl. In an exemplary embodiment, A¹ is H. In an exemplary embodiment, A¹ is methyl. In an exemplary embodiment, A¹ is ethyl.

In an exemplary embodiment, the compound has a structure according to the formula:

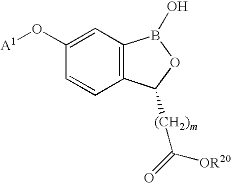

wherein A¹, m and R²⁰ are as defined herein.

In an exemplary embodiment, the compound has a structure according to the formula:

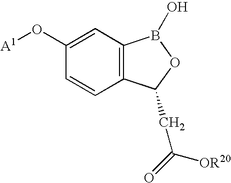

wherein A¹ and R²⁰ are as defined herein. In an exemplary embodiment, R²⁰ is H. In an exemplary embodiment, R²⁰ is methyl. In an exemplary embodiment, R²⁰ is ethyl. In an exemplary embodiment, A¹ is H. In an exemplary embodiment, A¹ is methyl. In an exemplary embodiment, A¹ is ethyl.

In another aspect, the invention provides a compound having a structure according to the formula:

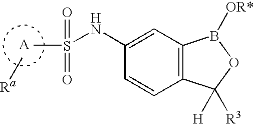

wherein R* is a member selected from H and a negative charge; A is a member selected from phenyl and pyridinyl; R³ is a member selected from H, cyano, substituted or unsubstituted nitroalkyl and substituted or unsubstituted aminoalkyl; R^{a} is a member selected from R¹⁰, OR¹⁰, NR¹⁰R¹¹, SR¹⁰, —S(O)R¹⁰, —S(O)₂R¹⁰, —S(O)₂NR¹⁰R¹¹, —C(O)R¹⁰, —C(O)OR¹⁰, —C(O)NR¹⁰R¹¹, wherein each R¹⁰ and each R¹¹ is a member independently selected from H, nitro, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, with the proviso that R^{a} is not H or unsubstituted alkyl or halosubstituted alkyl.

In another aspect, the invention provides a compound having a structure according to the formula:

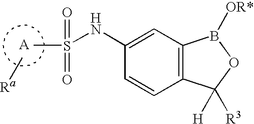

wherein R* is a member selected from H and a negative charge; A is a member selected from phenyl and pyridinyl; R³ is a member selected from H, cyano, substituted or unsubstituted nitroalkyl and substituted or unsubstituted aminoalkyl; R^{a} is a member selected from R¹², OR¹⁰, NR¹⁰R¹¹, SR¹⁰, —S(O)R¹⁰, —S(O)₂R¹⁰, —S(O)₂NR¹⁰R¹¹, —C(O)R¹⁰, —C(O)OR¹⁰, —C(O)NR¹⁰R¹¹, wherein each R¹⁰ and each R¹¹ is a member independently selected from H, nitro, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, and wherein R¹² is a member selected from nitro, cyano, alkyl, heteroalkyl, cycloalkyl, heterocycloalkyl, aryl and heteroaryl wherein said alkyl is substituted by one or more groups selected from OR¹³, NR¹³R¹⁴, SR¹³, —S(O)R¹³, —S(O)₂R¹³, —S(O)₂NR¹³R¹⁴, —C(O)R¹³, —C(O)OR¹³ and —C(O)NR¹³R¹⁴, wherein each R¹³ and each R¹⁴ is a member independently selected from H, nitro, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl; and wherein said heteroalkyl, cycloalkyl, heterocycloalkyl, aryl, heteroaryl are optionally substituted with one or more groups selected from R¹⁵, OR¹⁵, NR¹⁵R¹⁶, SR¹⁴, —S(O)R¹⁵, —S(O)₂R¹⁵, —S(O)₂NR¹⁵R¹⁶, —C(O)R¹⁵, —C(O)OR¹⁵, —C(O)NR¹⁵R¹⁶, wherein each R¹⁵ and each R¹⁶ is a member independently selected from H, nitro, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In an exemplary embodiment, R^{a} is a member selected from —Y¹R⁵, —CN, —R⁴Y², —C(O)OR⁶, —NH₂ and OH. Y¹ is a member selected from O and S. Y² is a member selected from NH₂ and OH. R⁴ is a member selected from substituted or unsubstituted alkylene and substituted or unsubstituted heteroalkylene. R⁵ is a member selected from H, substituted or unsubstituted alkyl. R⁶ is a member selected from H, substituted or unsubstituted alkyl and substituted or unsubstituted heteroalkyl.

In an exemplary embodiment, the invention has a structure according to the formula:

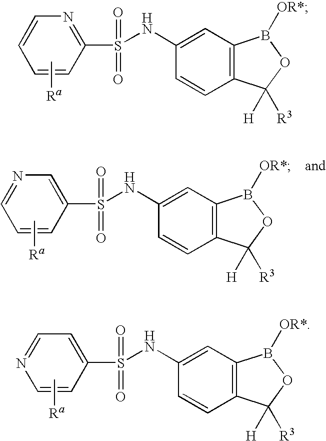

In an exemplary embodiment, the compound has a structure according to the following formula:

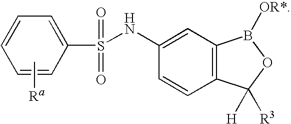

In an exemplary embodiment, the invention has a structure according to the following formula:

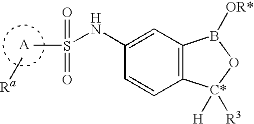

wherein C* is a carbon atom, with the proviso that when R³ is not H, C* is a stereocenter which has a configuration which is a member selected from (R) and (S). In an exemplary embodiment, the invention has a structure according to the following formula:

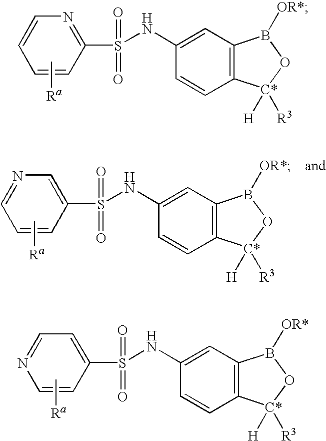

wherein C* is a carbon atom, with the proviso that when R³ is not H, C* is a stereocenter which has a configuration which is a member selected from (R) and (S). In an exemplary embodiment, the compound has a structure according to the following formula:

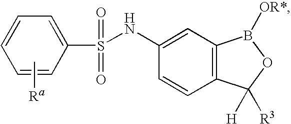

wherein R^{a} is a member selected from —NH₂, —CN, —OR⁵, —COOR⁵, —R⁴NH₂ and —R⁴OH, wherein R⁴ is unsubstituted alkylene and R⁵ is substituted or unsubstituted alkyl. In an exemplary embodiment, R³ is H, R^{a} is a member selected from —NH₂, —NO₂, —CN, —OCH₃, —OCF₃, —COOH, —CH₂NH₂ and —CH₂OH. In an exemplary embodiment, the compound is a member selected from

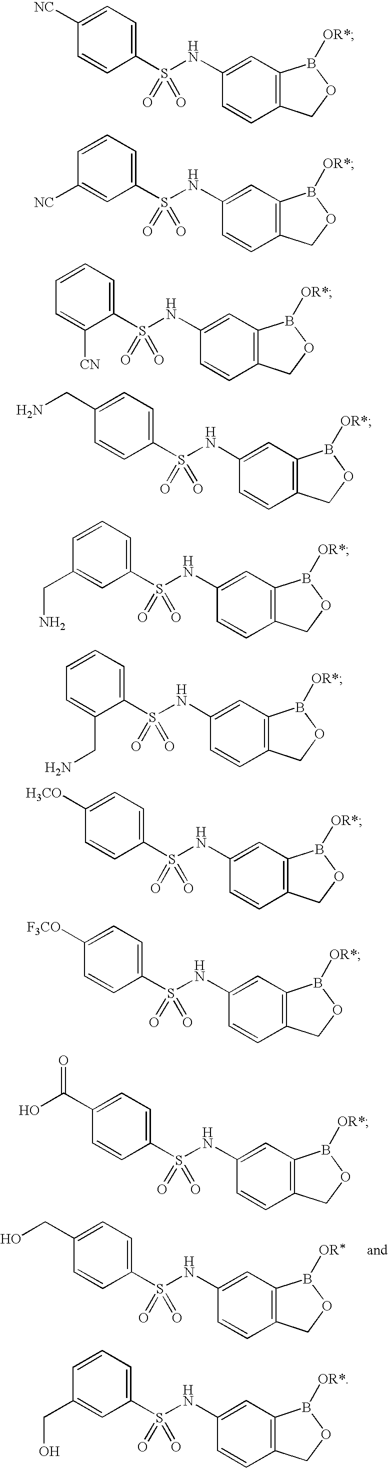

In an exemplary embodiment, R³ is —(CR²⁰R²¹)ₙNR²²R²³ in which the index n is an integer selected from 1 to 10; each R²⁰ and each R²¹ is a member independently selected from H, R²⁶, OR²⁶, NR²⁶R²⁷, SR²⁶, —S(O)R²⁶, —S(O)₂R²⁶, —S(O)₂NR²⁶R²⁷, —C(O)R²⁷, —C(O)OR²⁷, —C(O)NR²⁶R²⁷; R²² and R²³ are members independently selected from H, —S(O)R²⁸, —S(O)₂R²⁸, —S(O)₂NR²⁸R²⁹, —C(O)R²⁸, —C(O)OR²⁸, —C(O)NR²⁸R²⁹, nitro, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl wherein each R²⁶, each R²⁷, each R²⁸ and each R²⁹ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, n is an integer selected from 1 to 5. In an exemplary embodiment, n is 1. In an exemplary embodiment, R²⁰ is substituted or unsubstituted alkyl. In an exemplary embodiment, R²⁰ is unsubstituted alkyl. In an exemplary embodiment, R²⁰ is C₁-C₄ unsubstituted alkyl. In an exemplary embodiment, R²⁰ is methyl. In an exemplary embodiment, R²¹ is H. In an exemplary embodiment, R²³ is H. In an exemplary embodiment, R³ is a member selected from cyano and —CH₂NO₂. In an exemplary embodiment, R²² is a member selected from —C(O)R²⁸ and —C(O)OR²⁸. In an exemplary embodiment, R²⁸ is a member selected from substituted or unsubstituted alkyl, substituted or unsubstituted heteroalkyl and substituted or unsubstituted aryl. In an exemplary embodiment, R²⁸ is a member selected from —(CR³⁰R³¹)ₘR³², wherein R³² is a member selected from substituted or unsubstituted aryl, —NR³³R³⁴ and OR³³, wherein the index m is an integer selected from 0 to 10; each R³³ and each R³⁴ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment R²⁸ is a member selected from

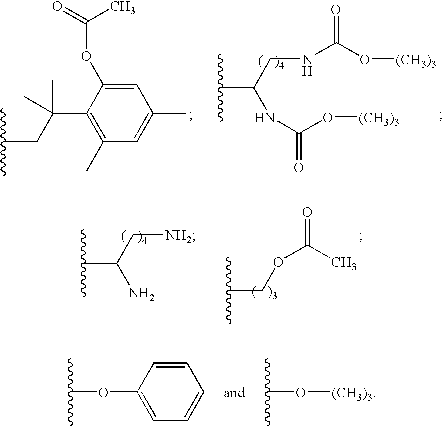

In another exemplary embodiment, the compound is

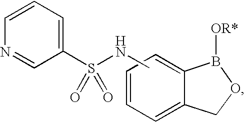

wherein R* is as described herein. In another exemplary embodiment, the compound is a member selected from

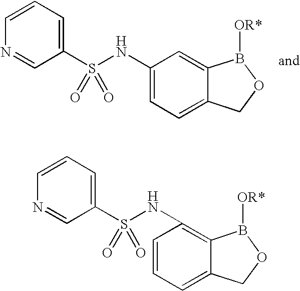

wherein R* is as described herein.

In another exemplary embodiment, the compound is a member selected from

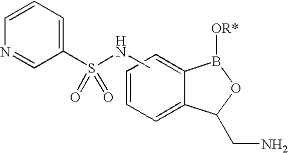

wherein R* is as described herein. In another exemplary embodiment, the compound is a member selected from

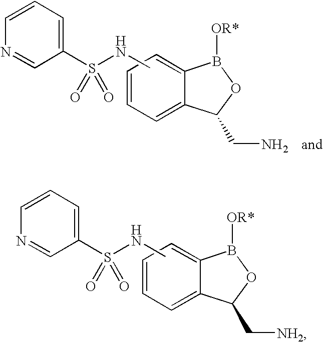

wherein R* is as described herein. In another exemplary embodiment, the compound is a member selected from

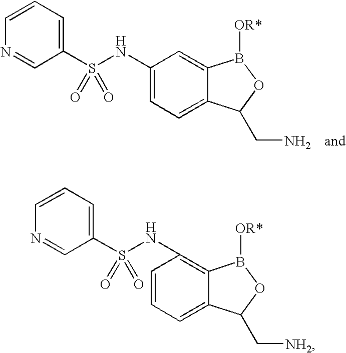

wherein R* is as described herein.

In another exemplary embodiment, the compound is a member selected from

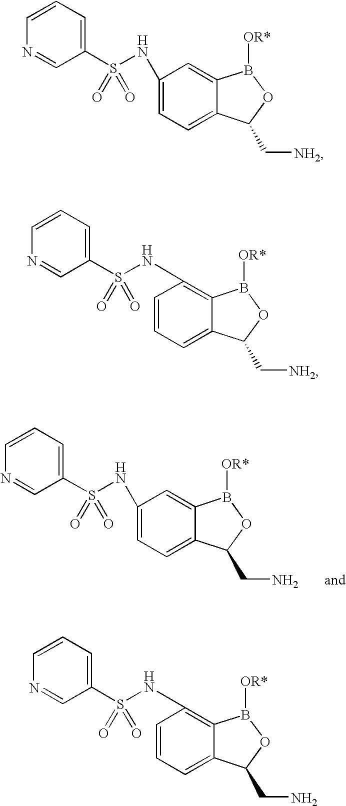

wherein R* is as described herein.

In another exemplary embodiment, the compound is

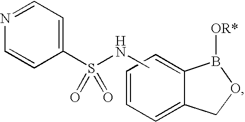

wherein R* is as described herein. In another exemplary embodiment, the compound is a member selected from

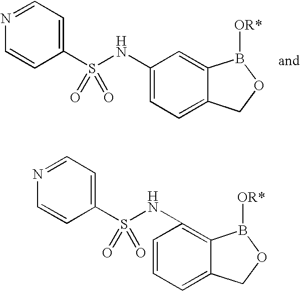

wherein R* is as described herein.

In another exemplary embodiment, the compound is a member selected

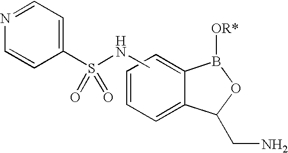

wherein R* is as described herein. In another exemplary embodiment, the compound is a member selected from

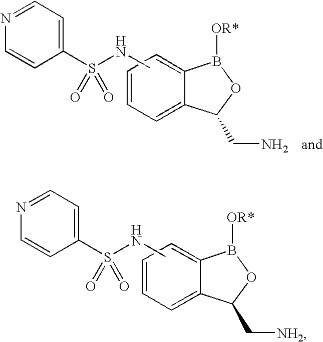

wherein R* is as described herein. In another exemplary embodiment, the compound is a member selected from

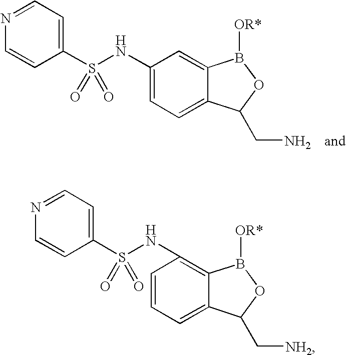

wherein R* is as described herein.

In another exemplary embodiment, the compound is a member selected from

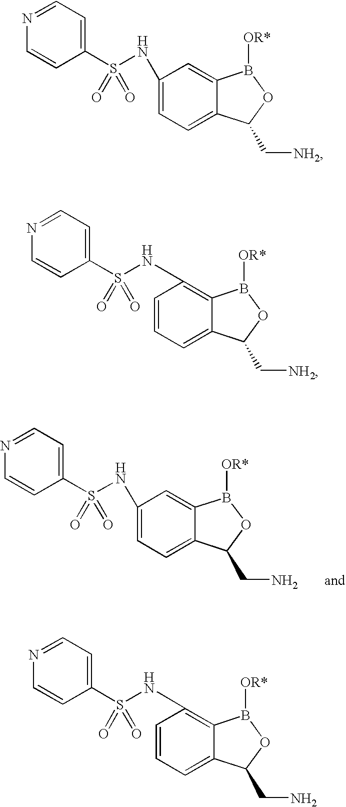

wherein R* is as described herein.

In an exemplary embodiment, R* is H. In an exemplary embodiment, the C* stereocenter is in a configuration which is a member selected from (R) and (S). In an exemplary embodiment, the C* stereocenter is in a (S) configuration. In an exemplary embodiment, the C* stereocenter is in a (S) configuration and R³ is —CH₂NH₂.

In an exemplary embodiment, the compound has a structure according to the formula:

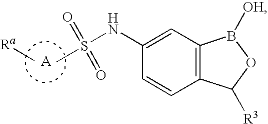

wherein A, R³ are as described herein, and R^{a} is as described herein, with the proviso that R^{a} is not H or unsubstituted alkyl or halosubstituted alkyl. In an exemplary embodiment, the compound has a structure according to the formula:

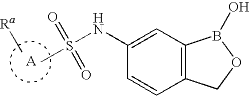

wherein R^{a} is as described herein, with the proviso that R^{a} is not H or unsubstituted alkyl or halosubstituted alkyl, and A is substituted phenyl or substituted pyridinyl or substituted or unsubstituted furanyl or substituted or unsubstituted thiophenyl or substituted or unsubstituted pyrazolyl or substituted or unsubstituted imidazolyl or substituted or unsubstituted thiazolyl or substituted or unsubstituted triazolyl or substituted or unsubstituted piperidinyl. In an exemplary embodiment, A is substituted phenyl or substituted pyridinyl or furanyl or thiophenyl or pyrazolyl or imidazolyl or substituted or unsubstituted thiazolyl or triazolyl or piperidinyl. In an exemplary embodiment, A is substituted phenyl. In an exemplary embodiment, A is substituted pyridin-2-yl or substituted pyridin-3-yl or substituted pyridin-4-yl.

In an exemplary embodiment, the compound has a formula which is a member selected from:

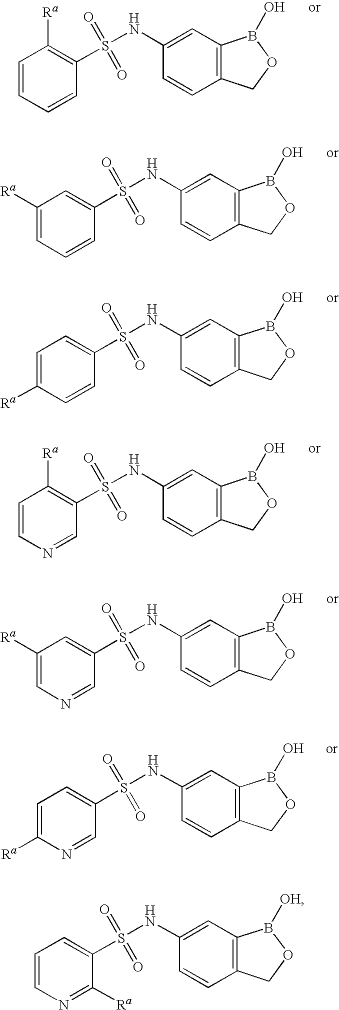

wherein R^{a} is a member selected from OR²⁰, NR²⁰R²¹, SR²⁰, —S(O)R²⁰, —S(O)₂R²⁰, —S(O)₂NR²⁰R²¹, —C(O)R²⁰, —C(O)OR²⁰ , —C(O)NR²⁰R²¹, nitro, cyano, substituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, each R²⁰ and each R²¹ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, with the proviso that R^{a} is not halosubstituted alkyl.

In an exemplary embodiment, the compound is a member selected from

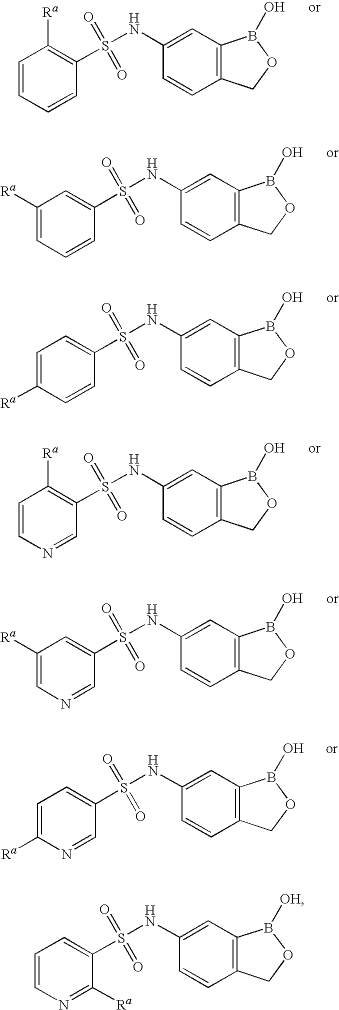

wherein R^{a} is a member selected from cyano, nitro, aminoalkyl, hydroxyalkyl, —C(O)(CH₂)ₙCH₃, COOH, —C(O)O(CH₂)ₙCH₃, O(CH₂)ₙCH₃, O(CH₂)ₙCF₃, O(CH₂)ₙCHF₂, OH, NH₂, NHCH₃, NHC(O)H, NHC(O)(CH₂)ₙCH₃, NHOH, NHS(O)₂NH₂, —NH₂S(O)₂CH₃, —S(O)₂CH₃, wherein n is 0 or 1 or 2 or 3.

In an exemplary embodiment, the compound is:

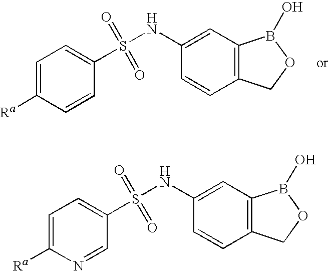

wherein R^{a1} is as described herein, or a salt thereof.

In an exemplary embodiment, the compound is:

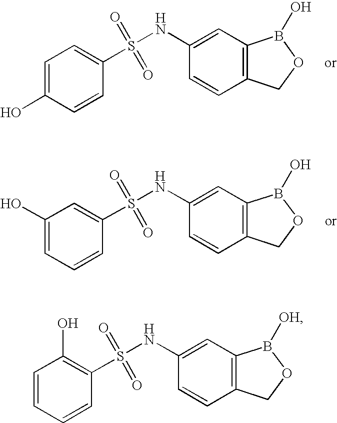

or a salt thereof.

In an exemplary embodiment, the compound is:

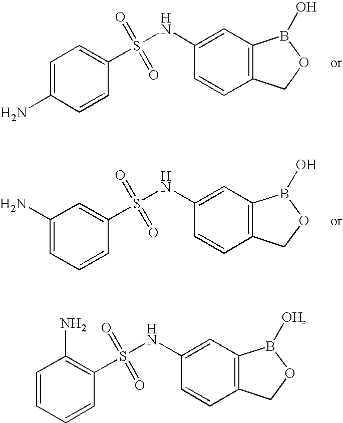

or a salt thereof.

In an exemplary embodiment, the compound is:

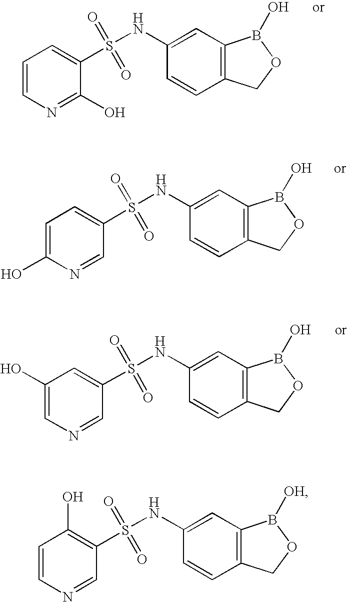

or a salt thereof.

In an exemplary embodiment, the compound is:

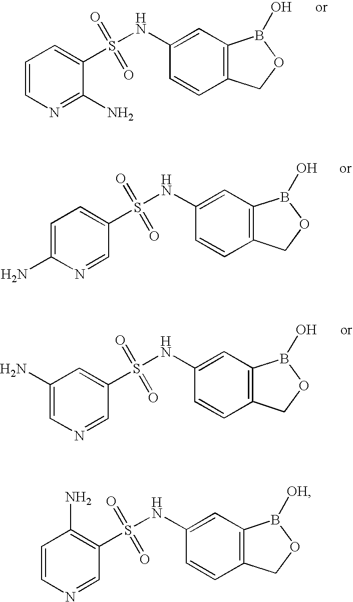

or a salt thereof.

In an exemplary embodiment, the compound is E111 or a salt thereof. In an exemplary embodiment, the compound is E111 or a pharmaceutically acceptable salt thereof. In an exemplary embodiment, the compound is E119 or a salt thereof. In an exemplary embodiment, the compound is E119 or a pharmaceutically acceptable salt thereof.

In an exemplary embodiment, the compound has a structure according to the following formula:

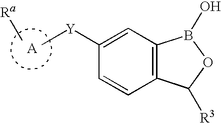

where R^{a}, A, R³ and Y are as described herein, with the proviso that when R³ is H or —CH₃ or —CH₂CH₃ or benzyl,

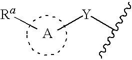

is not a member selected from

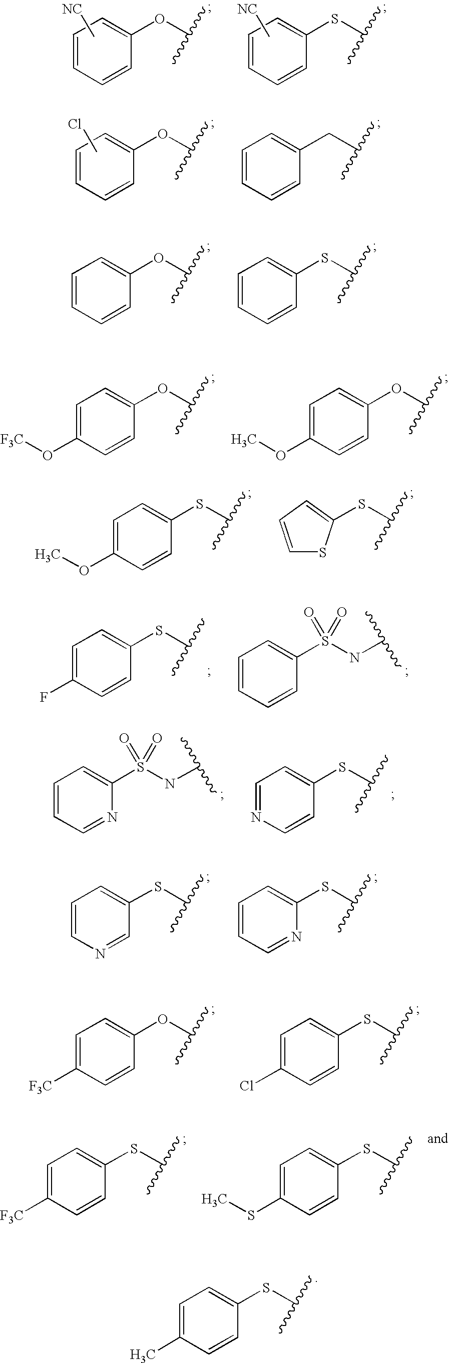

In an exemplary embodiment, the compound has a structure according to the following formula:

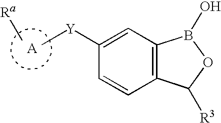

where R^{a}, A, R³ and Y are as described herein, with the proviso that when R³ is H,

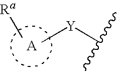

is not a member selected from

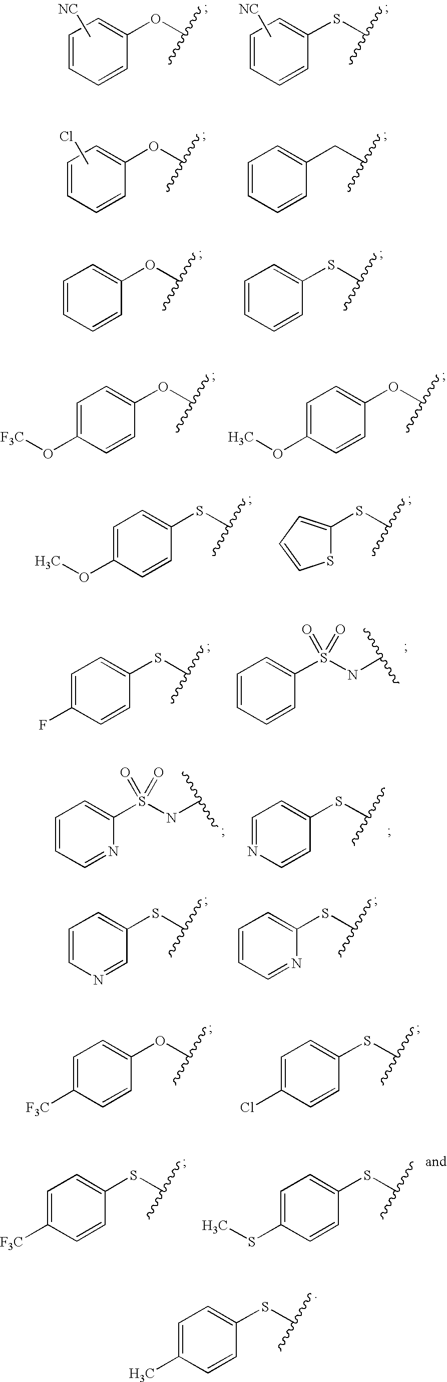

In an exemplary embodiment, the compound has a structure according to the following formula:

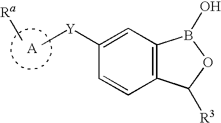

where R^{a}, A, R³ and Y are as described herein, with the proviso that when R³ is —CH₃ or —CH₂CH₃ or benzyl,

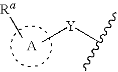

is not a member selected from

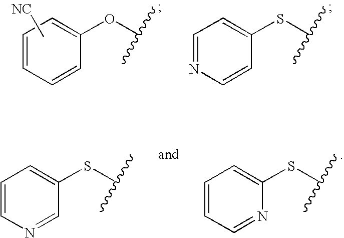

In an exemplary embodiment, there is the proviso that when R³ is —CH₃ or benzyl,

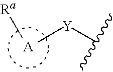

is not a member selected from

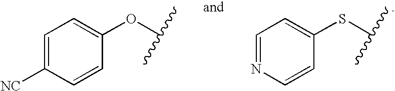

In an exemplary embodiment, there is the further proviso that when R³ is H,

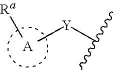

is not a member selected from

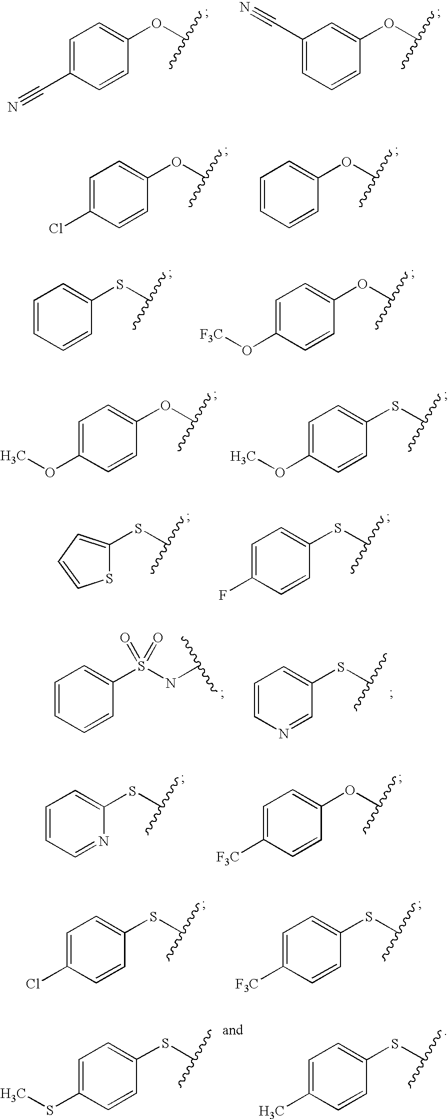

In an exemplary embodiment, there is a proviso that R^{a} is not cyano, halogen, H, —SCH₃, —OCH₃, —OCF₃, —CF₃, and —CH₃. In an exemplary embodiment, there is a proviso that R^{a} is not cyano, halogen, H, —SCH₃, —SCH₂CH₃, —OCH₃, —OCH₂CH₃, —OCF₃, —OCH₂CF₃, —CF₃, —CH₂CF₃, —CH₃ and —CH₂CH₃. In an exemplary embodiment, there is a proviso that when Y is O and A is phenyl, R^{a} is not cyano, chloro, H, —OCF₃, —OCH₃, —CF₃. In an exemplary embodiment, there is a proviso that when Y is S and A is phenyl, R^{a} is not halo, H, —OCF₃, —OCH₃, —SCH₃, —CF₃, —CH₃. In an exemplary embodiment, there is a proviso that when Y is S and A is pyridinyl or thiazolyl, R^{a} is not H. In an exemplary embodiment, there is a proviso that when Y is —S(O)₂NH— and A is phenyl, R^{a} is not H. In an exemplary embodiment, there is a proviso that when Y is O and A is phenyl, R^{a} is not cyano, chloro, H, —OCF₃, —OCH₂CF₃, —OCH₃, —OCH₂CH₃, —CF₃ and —CH₂CF₃. In an exemplary embodiment, there is a proviso that when Y is S and A is phenyl, R^{a} is not halo, H, —OCH₂CF₃, —OCH₃, —OCH₂CH₃, —SCH₃, —SCH₂CH₃, —CF₃, —CH₂CF₃, —CH₃ and —CH₂CH₃. In an exemplary embodiment, there is a proviso that when Y is S, then A is not pyridinyl. In an exemplary embodiment, there is a proviso that when Y is S, then A is not thiazolyl.

In an exemplary embodiment, R³ is not —CH₂—Ph. In an exemplary embodiment, Y is O. In an exemplary embodiment, A is a member selected from substituted or unsubstituted phenyl, substituted or unsubstituted pyridinyl, substituted or unsubstituted pyrimidinyl, substituted or unsubstituted pyrazinyl, substituted or unsubstituted thiazolyl, substituted or unsubstituted cyclopropyl, substituted or unsubstituted cyclobutyl, substituted or unsubstituted cyclopentyl, substituted or unsubstituted cyclohexyl and substituted or unsubstituted piperidinyl. In an exemplary embodiment, A is a member selected from substituted or unsubstituted pyridin-2-yl, substituted or unsubstituted pyridin-3-yl and substituted or unsubstituted piperidin-4-yl.

In an exemplary embodiment, A is a member selected from

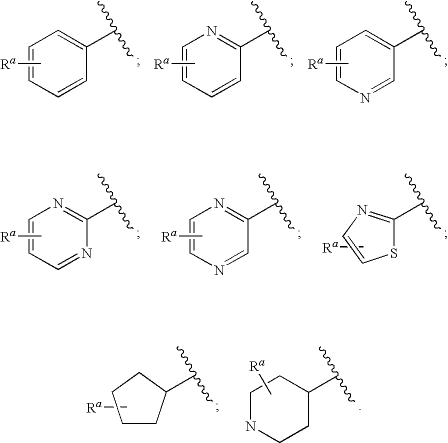

In an exemplary embodiment, A is a member selected from

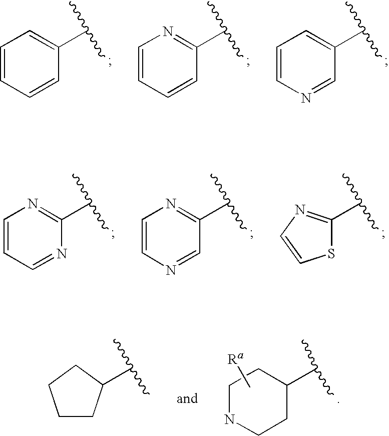

In an exemplary embodiment, R^{a} is H and A is a member selected from

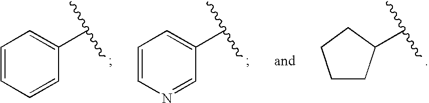

In an exemplary embodiment, A is a member selected from

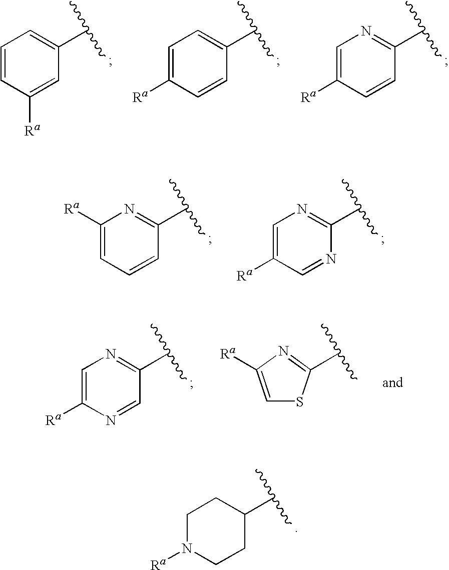

In an exemplary embodiment,

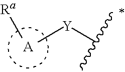

is a member

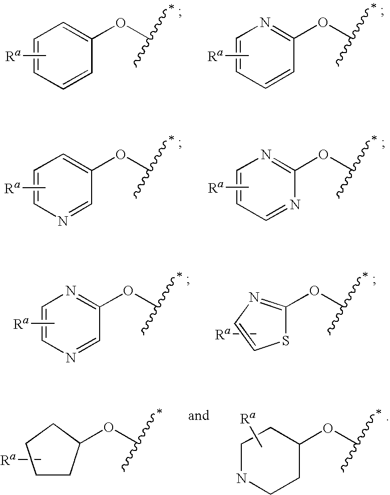

In an exemplary embodiment,

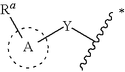

is a member selected from

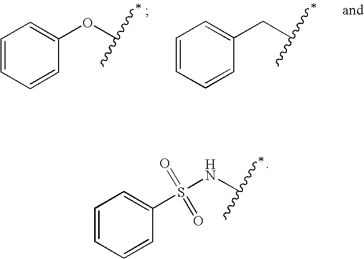

In an exemplary embodiment,

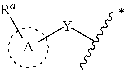

is a member selected from

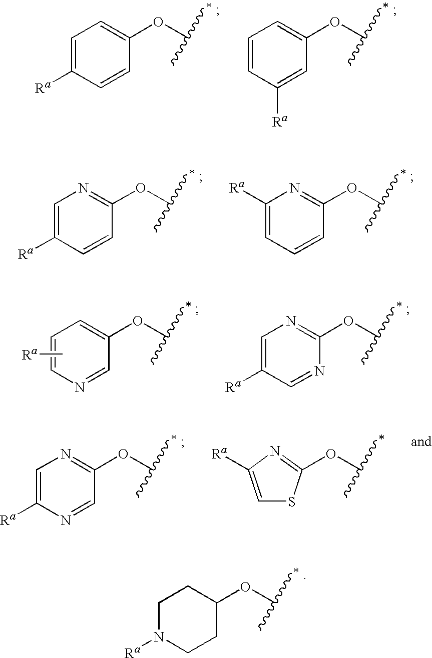

In an exemplary embodiment, R^{a} is cyano. In an exemplary embodiment, R^{a} is a member selected from aminomethyl, hydroxymethyl, —OH, —OCH₃, —NH₂, —NO₂, —C(O)OR²⁰, —C(O)NR²⁰R²¹ and

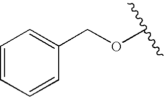

wherein each R²⁰ and each R²¹ is a member independently selected from H, methyl, ethyl, propyl, isopropyl, butyl, isobutyl, t-butyl, phenyl and benzyl. In an exemplary embodiment, R^{a} is a member selected from —C(O)OH, —C(O)OCH₃, —C(O)OCH₂CH₃, —C(O)OC(CH₃)₃ and —C(O)NH₂.

In an exemplary embodiment, R³ H. In an exemplary embodiment, R³ is a member selected from cyano. In an exemplary embodiment, R³ is substituted or unsubstituted C₁-C₃ alkyl.

In an exemplary embodiment, R³ is substituted with a member selected from —OH, —NH₂, nitro, —P(O)OR¹¹OR¹², —S(O)²R¹¹, —C(O)OR¹¹, —OSiR¹¹R¹²R¹³, —NHC(O)R¹¹, wherein each R¹¹, R¹² and R¹³ are members independently selected from H, —NH₂, NHR¹⁴ and substituted or unsubstituted alkyl, wherein R¹⁴ is —C(O)OR¹⁵, wherein R¹⁵ is unsubstituted alkyl.

In an exemplary embodiment, R³ is —CH₂R⁹, wherein R⁹ is a member selected from —OH, —NH₂, nitro, —P(O)OR²⁰OR²⁰, —S(O)₂R²², —C(O)OR²⁰, —OSiR²⁰R²¹R²², —NHC(O)R²⁰, wherein each R²⁰, each R²¹ and each R²² is a member independently selected from H, —NH₂, NHR¹⁴ and unsubstituted alkyl, wherein R¹⁴ is —C(O)OR¹⁵, wherein R¹⁵ is unsubstituted alkyl. In an exemplary embodiment, R³ is substituted with a member selected from —OH, —NH₂, nitro, —P(O)(OCH₃)₂, —S(O)₂CH₃, —S(O)₂CH₂CH₃, —S(O)₂NH₂, —S(O)₂NHC(O)C(CH₃)₃, —C(O)OH, —C(O)OCH₂CH₃, —OSi(CH₃)₂(C(CH₃)₃), —NHC(O)(CH₂)₂CH(CH₃)₂. In an exemplary embodiment, the alkyl group is a member selected from —CH₂OH, —(CH₂)₂OH, —(CH₂)₃OH, —CH₂NH₂, —CH₂NO₂, —CH₂P(O)(OCH₃)₂, —CH₂S(O)₂CH₃, —CH₂S(O)₂CH₂CH₃, —CH₂S(O)₂NH₂, —CH₂S(O)₂NHC(O)C(CH₃)₃, —CH₂C(O)OH, —CH₂C(O)OCH₂CH₃, —CH₂C(O)OCH₃, —(CH₂)₃OSi(CH₃)₂(C(CH₃)₃) and —CH₂NHC(O)(CH₂)₂CH(CH₃)₂. In an exemplary embodiment, R³ is substituted with —OH and nitro.

In an exemplary embodiment, Y is a member selected from unsubstituted C₁-C₄ alkyl and —S(O)₂NH—, R³ is a member selected from H, aminomethyl, hydrixymethyl, —OH, —OCH₃, —NH₂, —NO₂, —C(O)OR²⁰, —C(O)NR²⁰R²¹ and

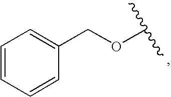

wherein each R²⁰ and each R²¹ is a member independently selected from H, methyl, ethyl, propyl, isopropyl, butyl, isobutyl, t-butyl, phenyl and benzyl.

In another exemplary embodiment, the invention provides poly- or multi-valent species of the compounds of the invention. In an exemplary embodiment, the invention provides a dimer of the compounds described herein. In an exemplary embodiment, the invention provides a dimer of the compounds described herein.

In an exemplary embodiment, the invention provides an anhydride of the compounds described herein. In an exemplary embodiment, the invention provides an anhydride of the compounds described herein.

In an exemplary embodiment, the invention provides a trimer of the compounds described herein. In an exemplary embodiment, the invention provides a trimer of the compounds described herein.

The compounds of the invention can form a hydrate with water, solvates with alcohols such as methanol, ethanol, propanol, and the like; adducts with amino compounds, such as ammonia, methylamine, ethylamine, and the like; adducts with acids, such as formic acid, acetic acid and the like; complexes with ethanolamine, quinoline, amino acids, and the like.

In an exemplary embodiment, the compound has a structure according to the formula:

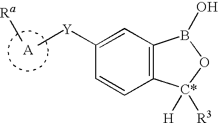

wherein R^{a}, A, and Y are as described herein, and R³ is not H, C* is a carbon atom which is a stereocenter which has a configuration of (R) or (S). In an exemplary embodiment, the C* stereocenter is in a (S) configuration. In an exemplary embodiment, the C* stereocenter is in a (S) configuration and R³ is —CH₂COOR²⁰, wherein R²⁰ is H or unsubstituted alkyl. In an exemplary embodiment, the C* stereocenter is in a (S) configuration and R³ is —CH₂COOH. In an exemplary embodiment, the C* stereocenter is in a (R) configuration. In an exemplary embodiment, the C* stereocenter is in a (R) configuration and R³ is —CH₂COOR²⁰, wherein R²⁰ is H or unsubstituted alkyl. In an exemplary embodiment, the C* stereocenter is in a (R) configuration and R³ is —CH₂COOH.

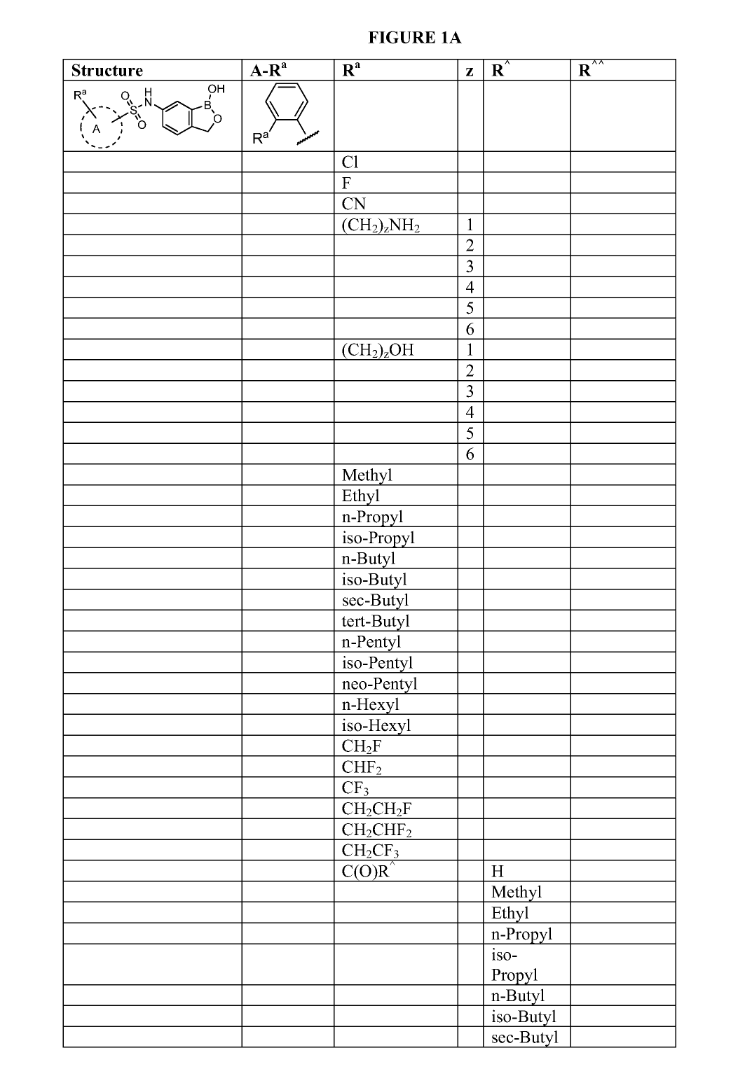

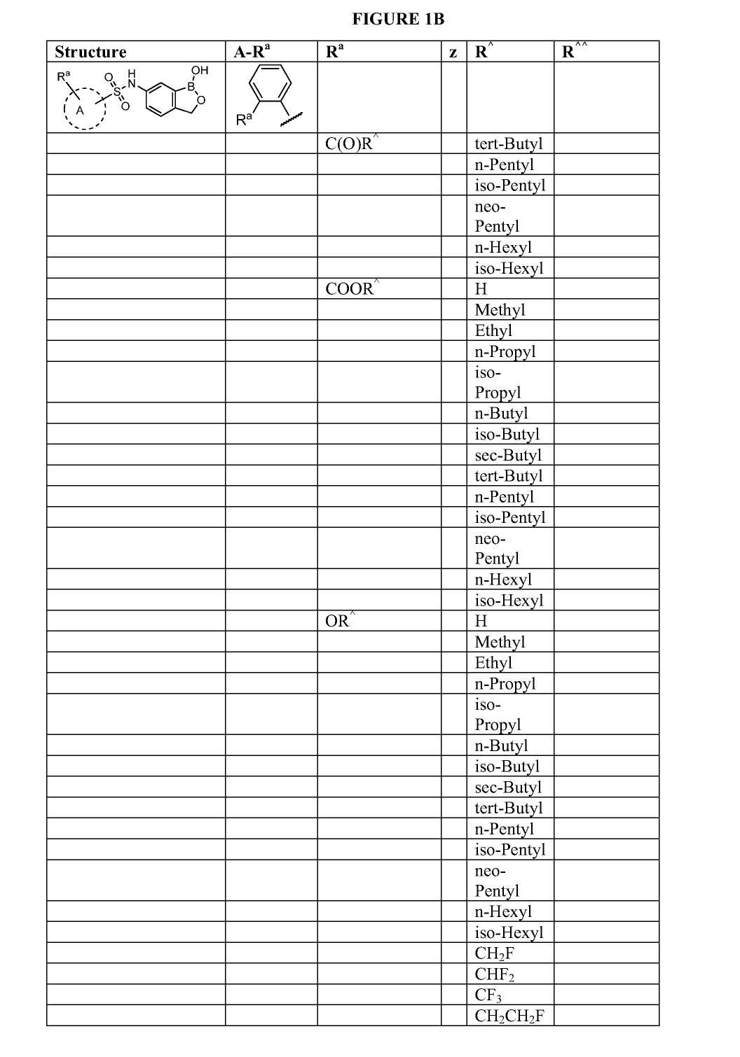

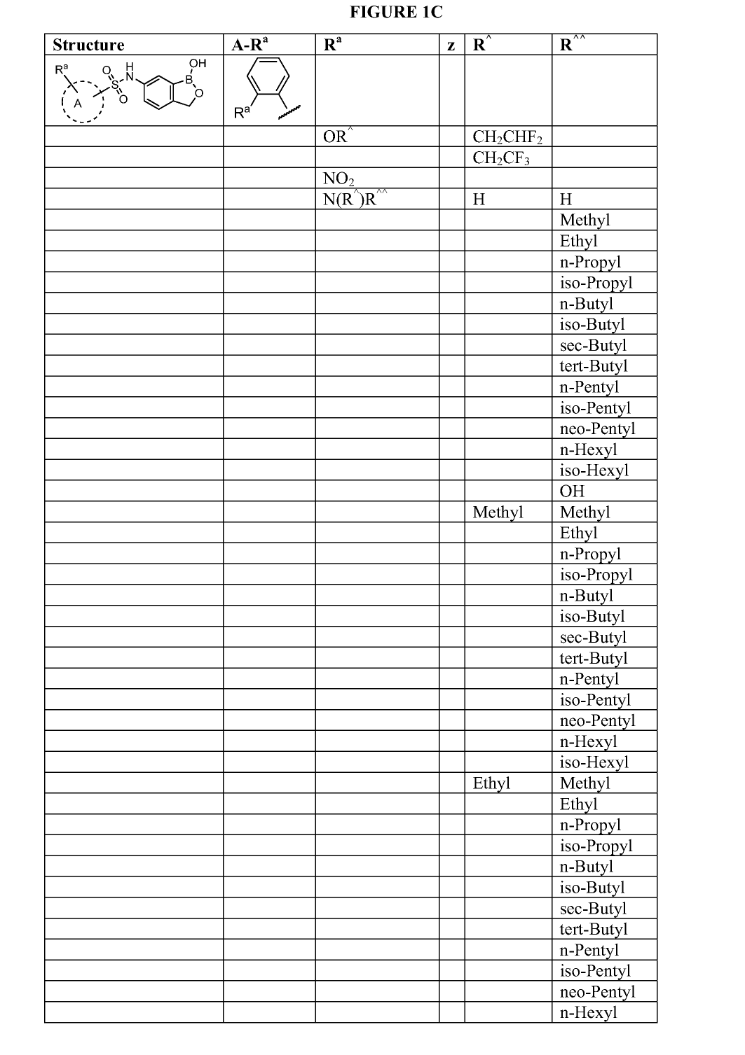

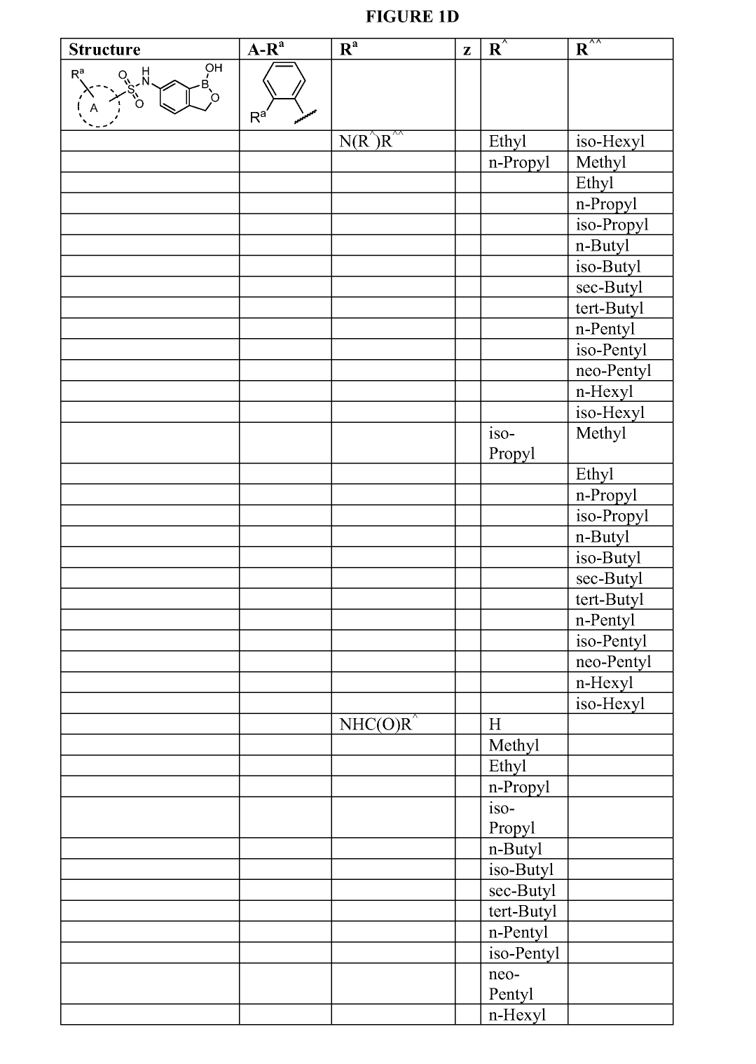

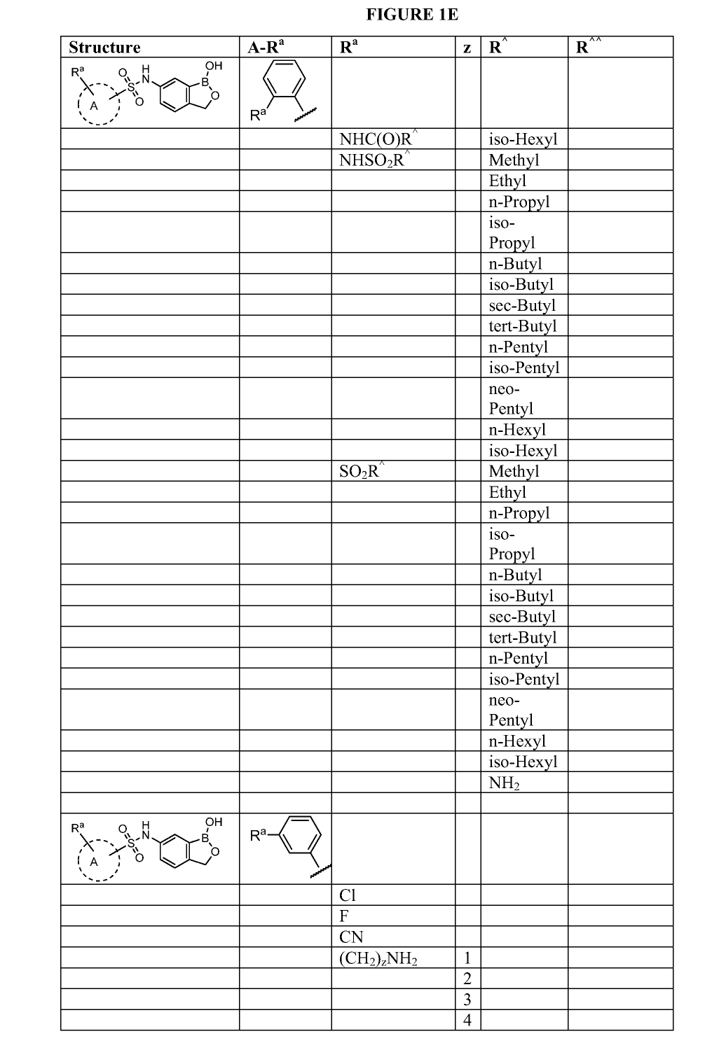

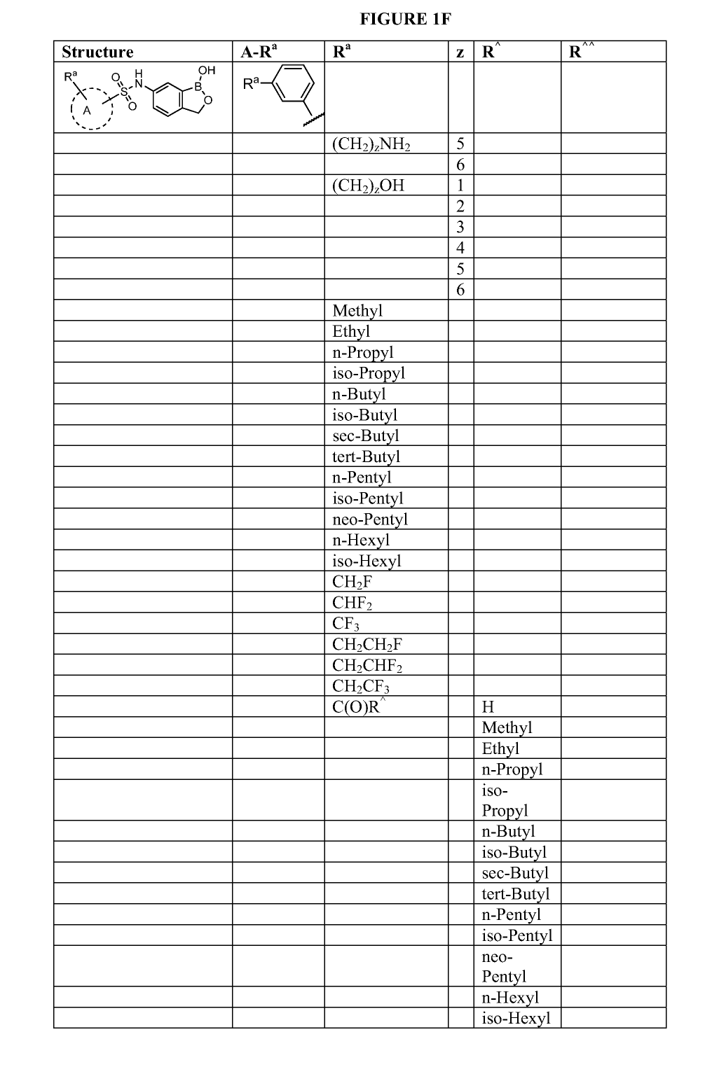

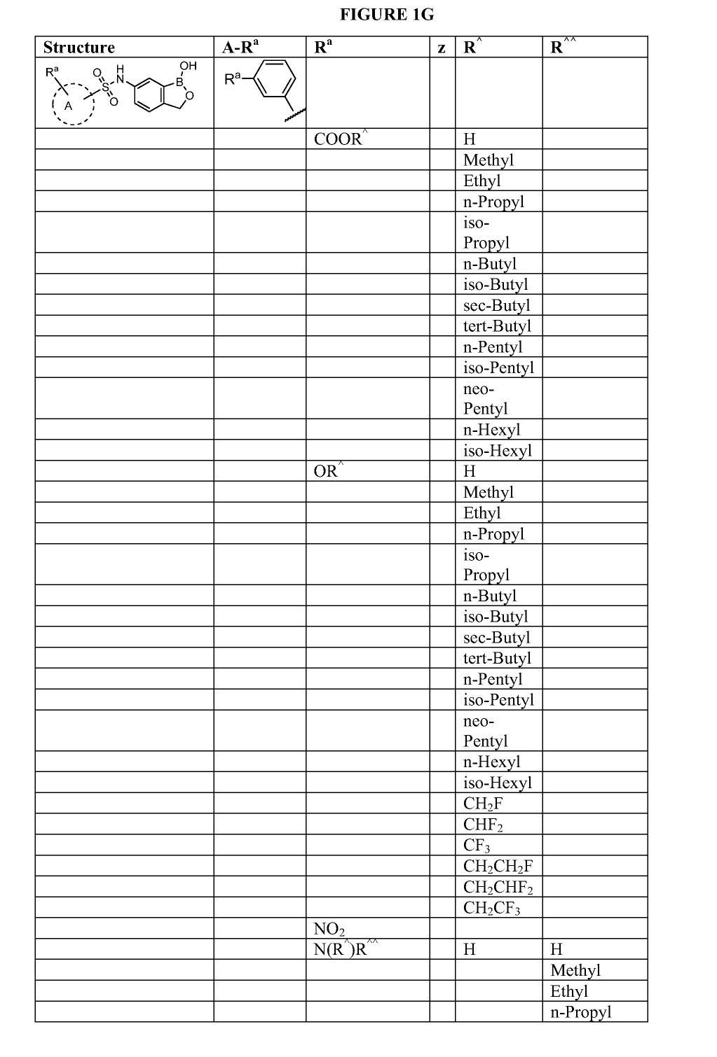

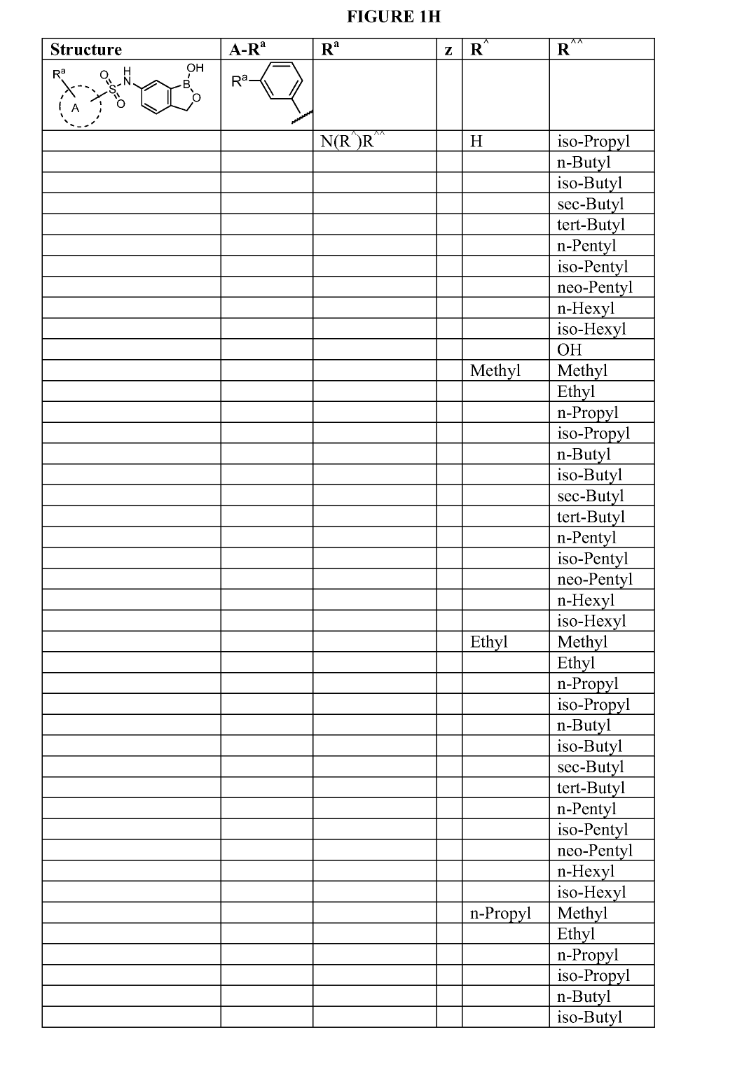

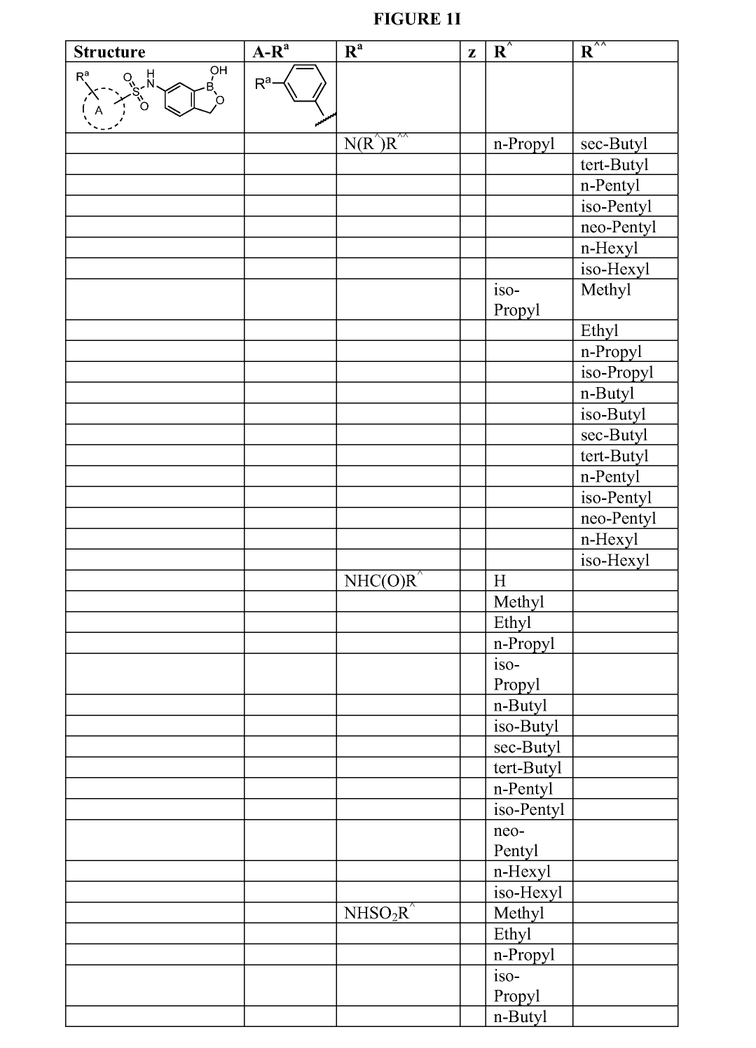

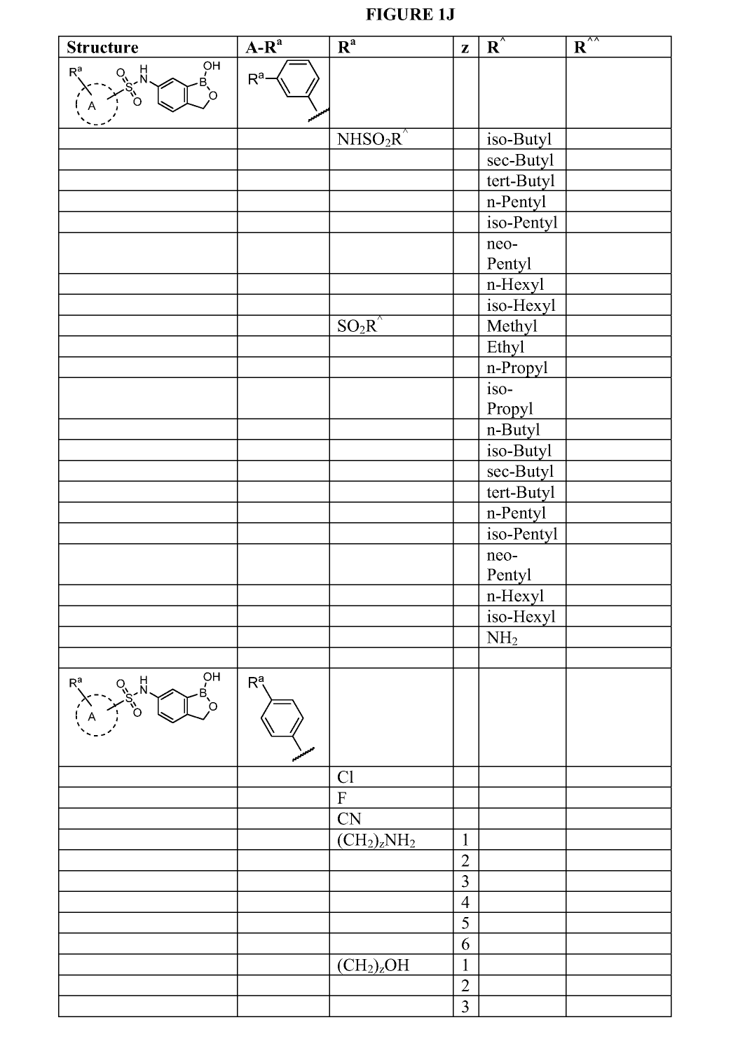

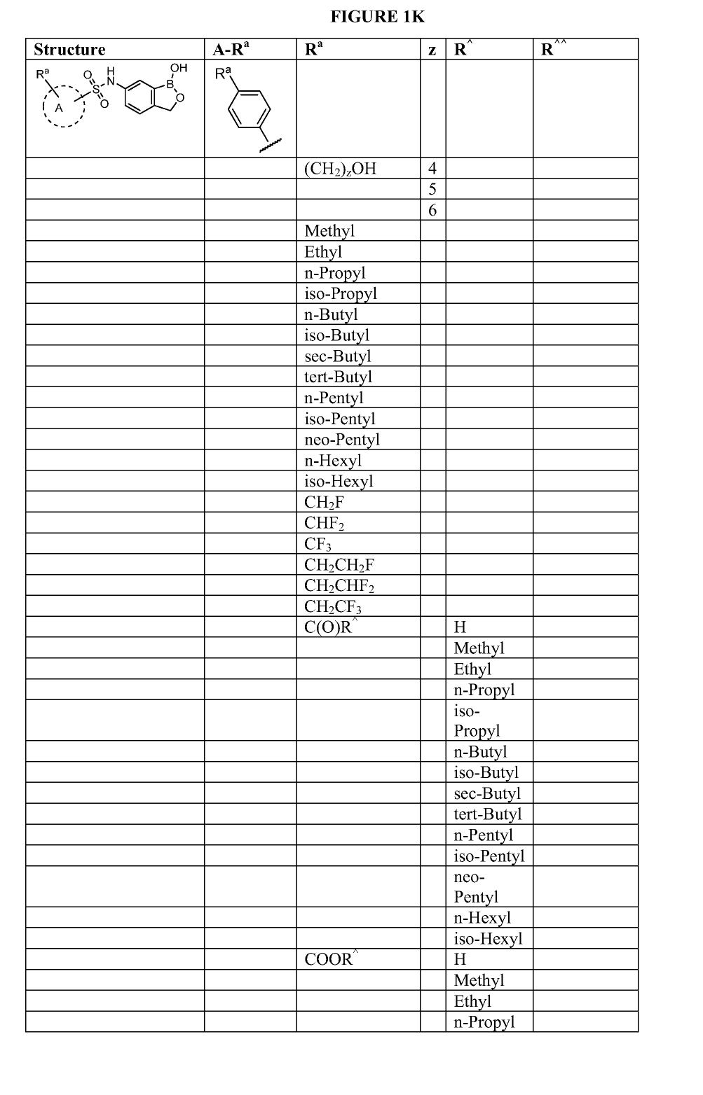

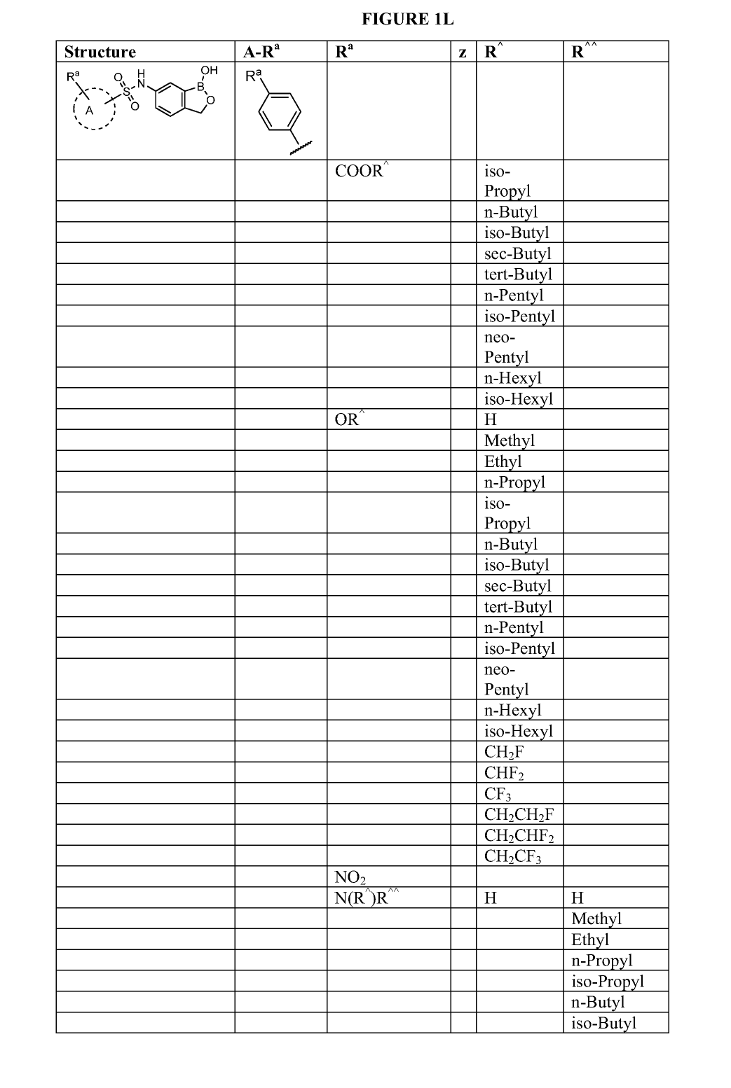

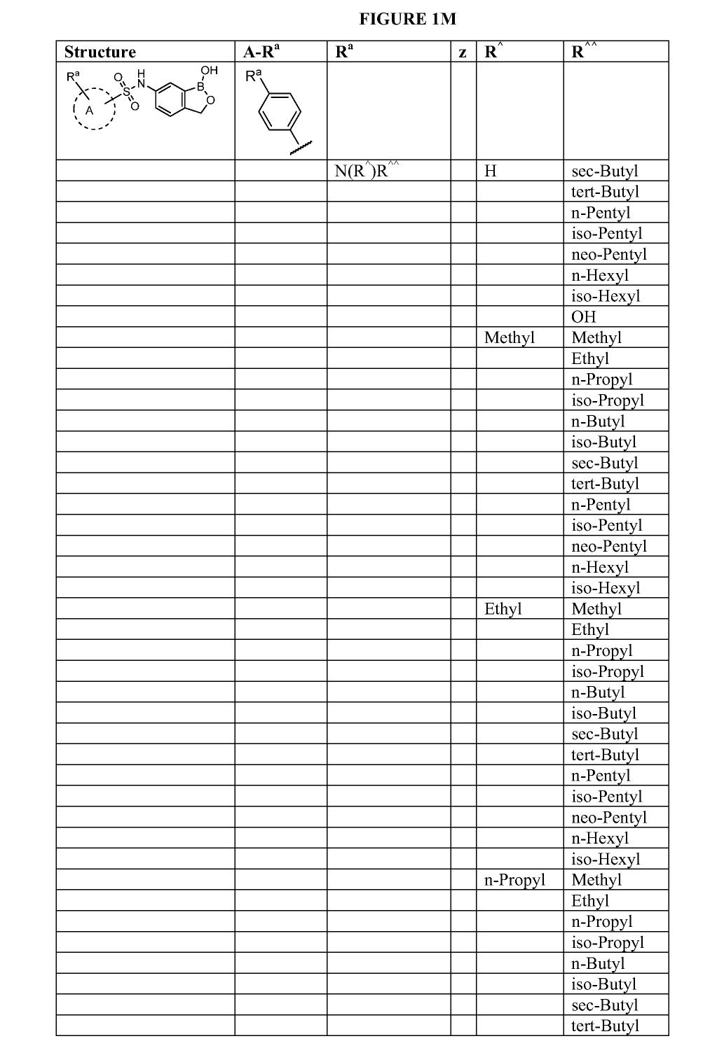

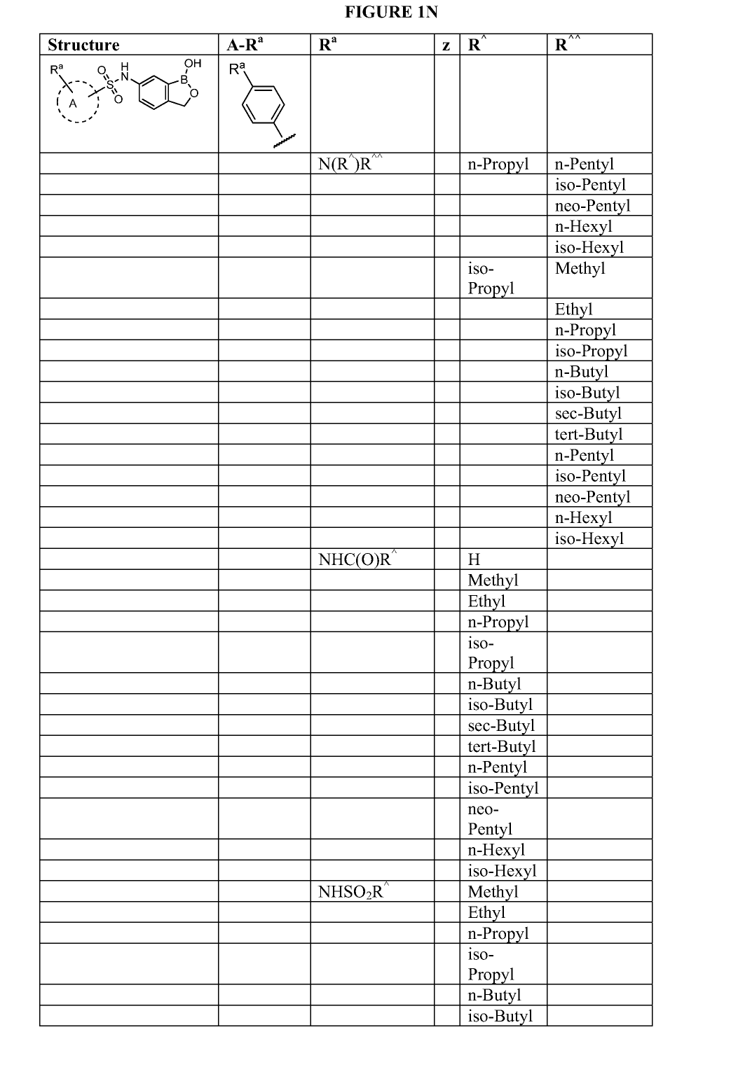

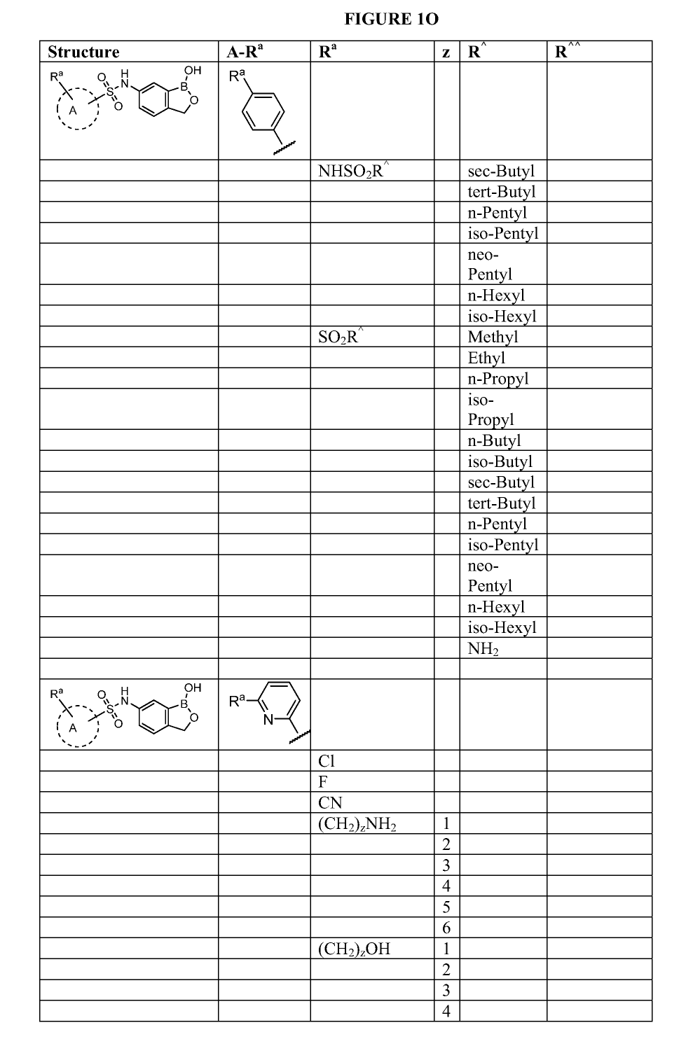

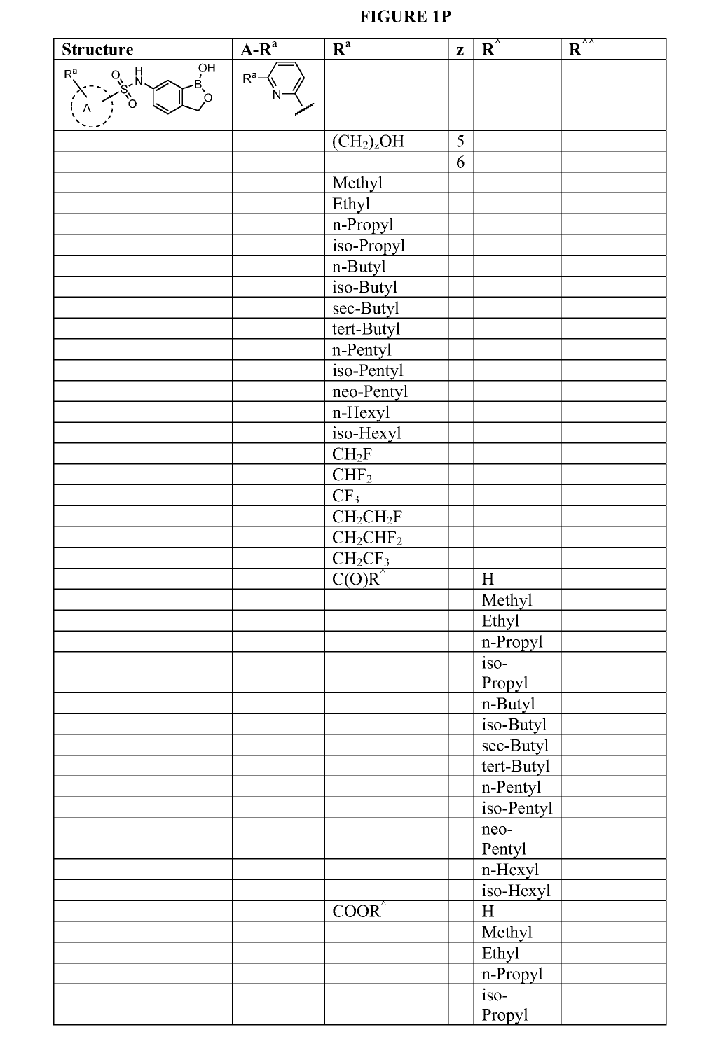

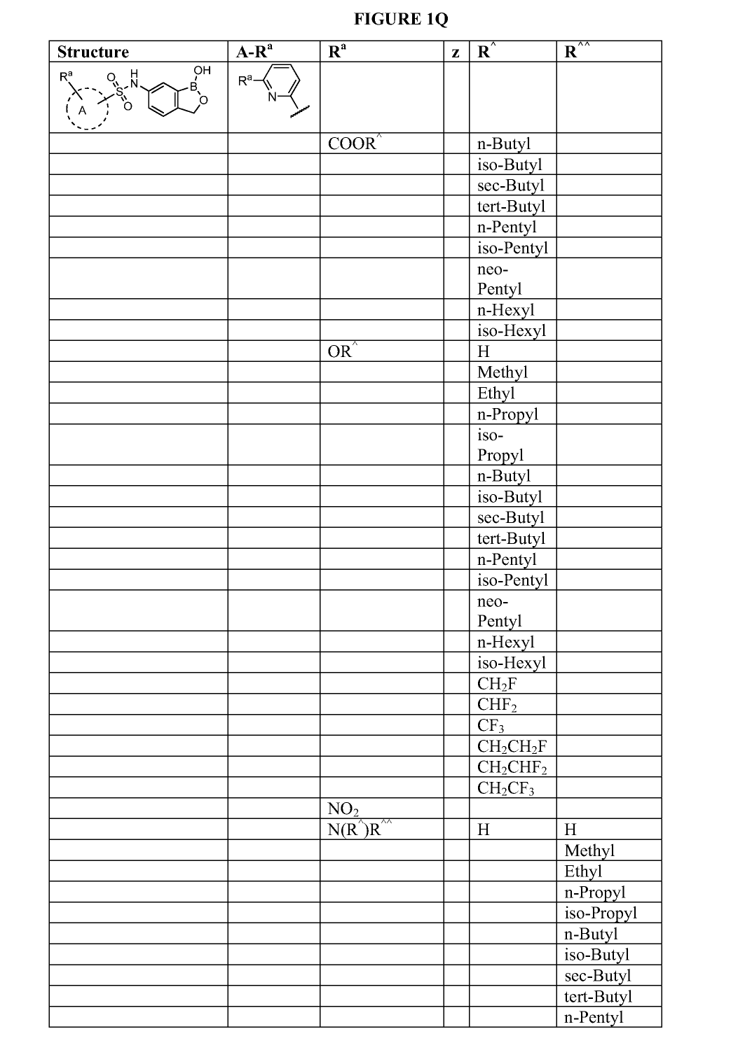

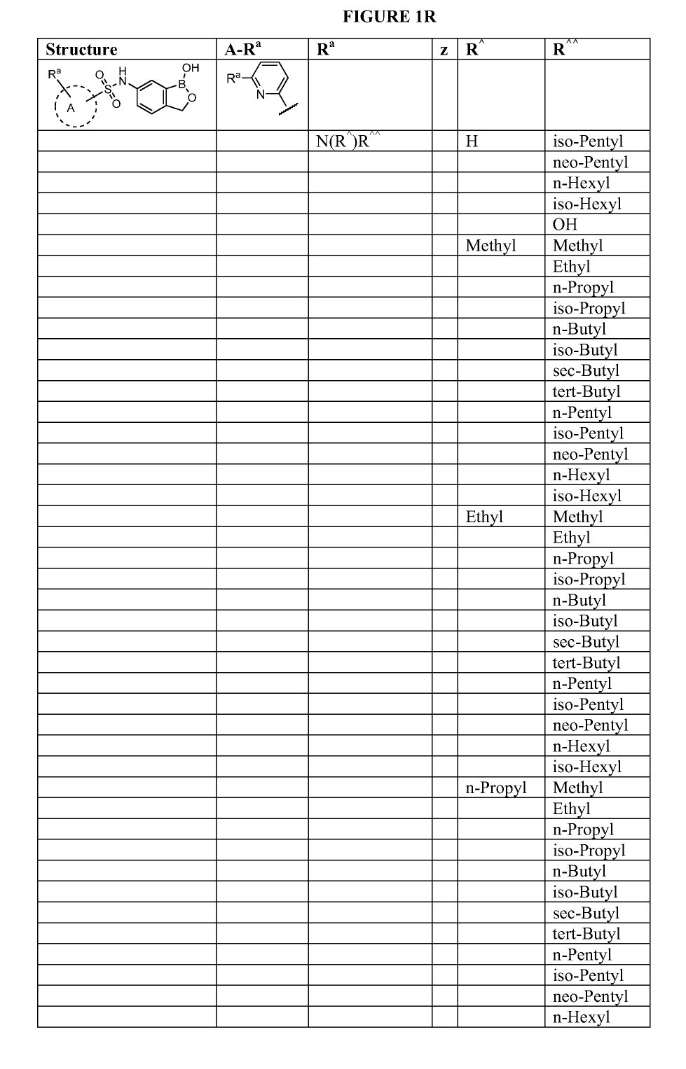

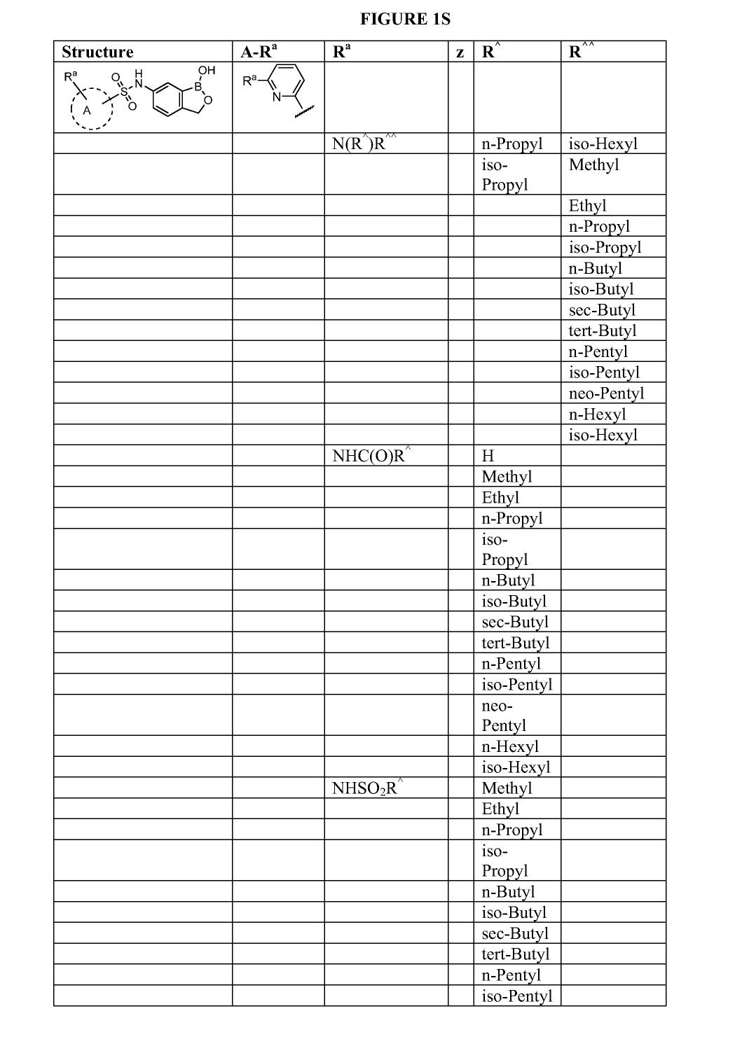

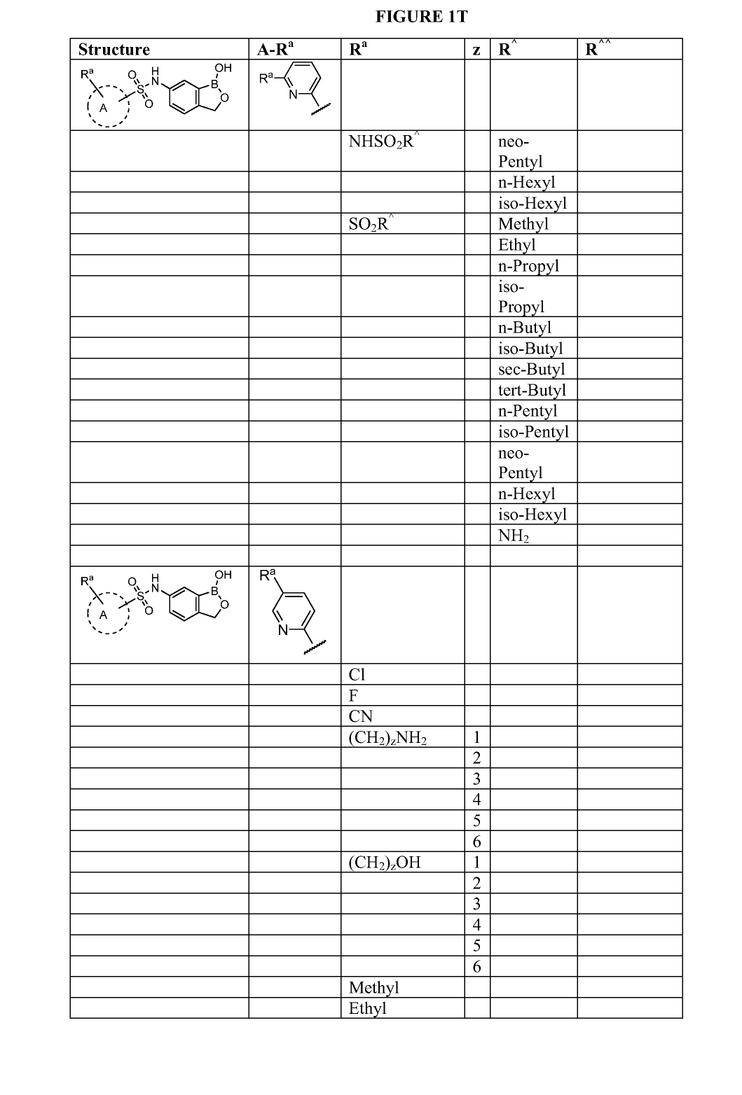

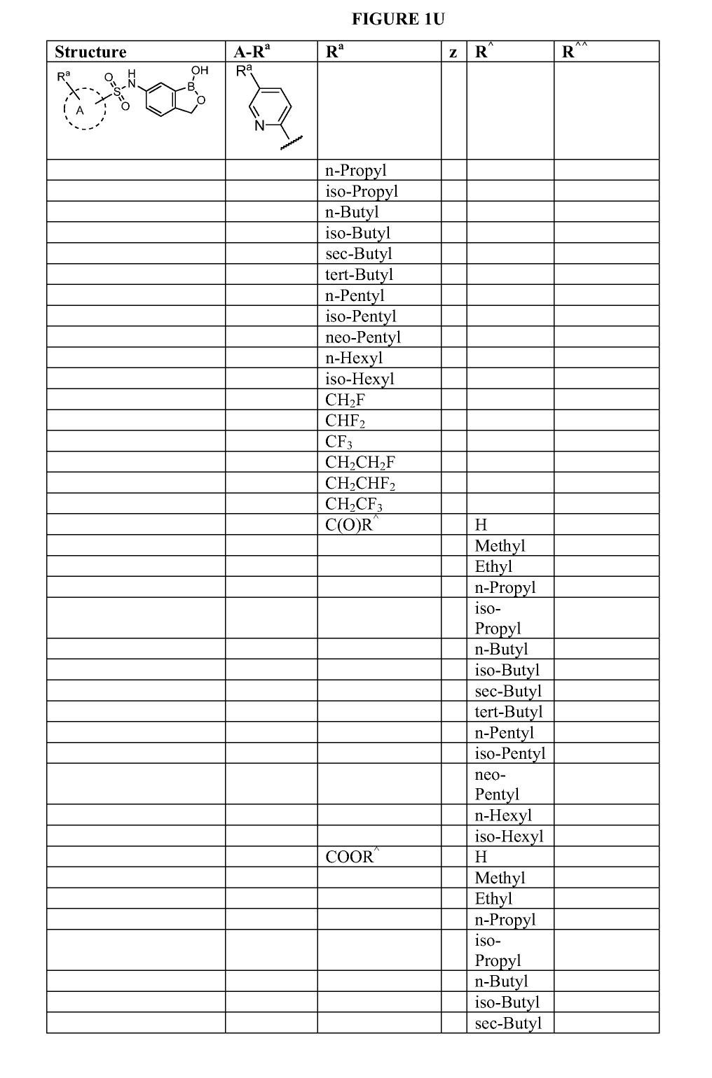

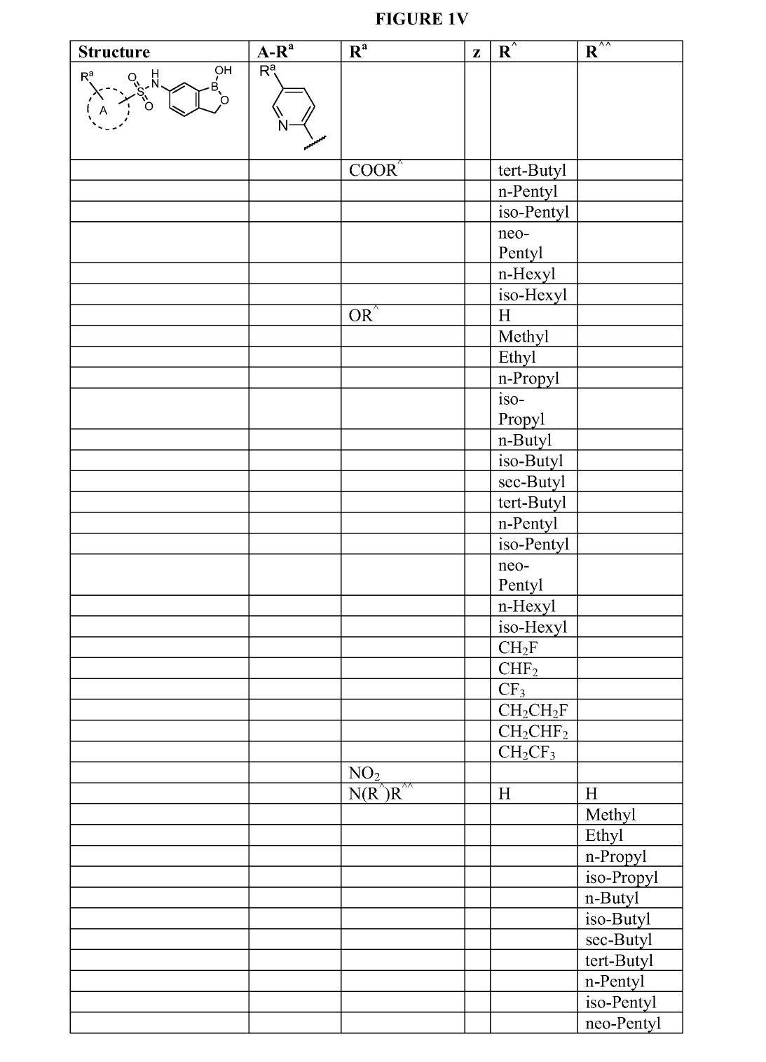

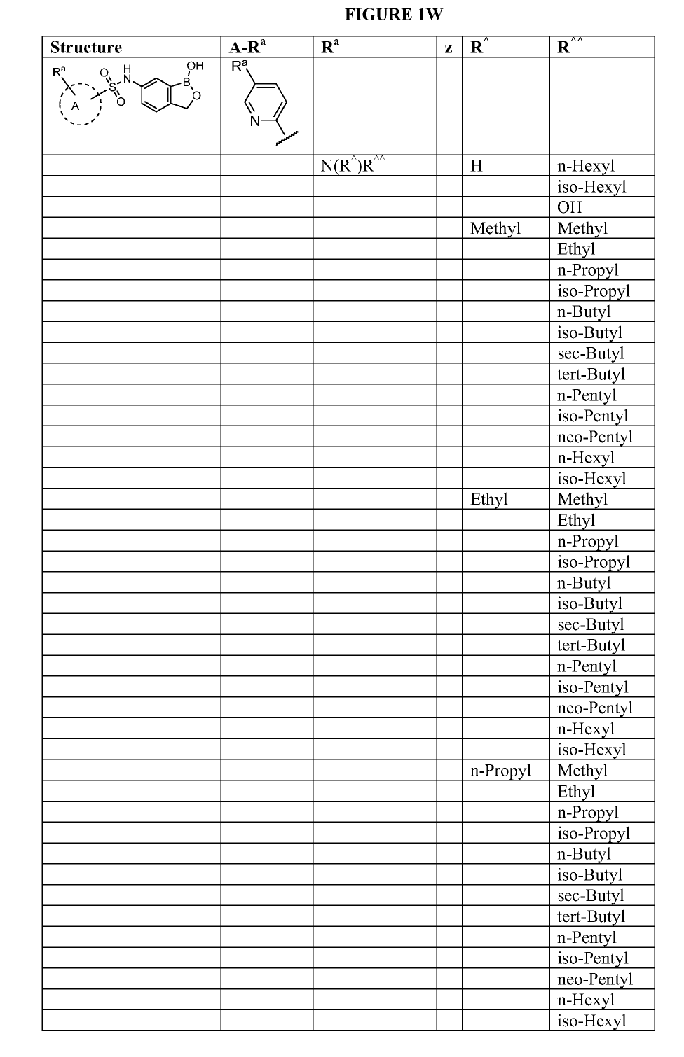

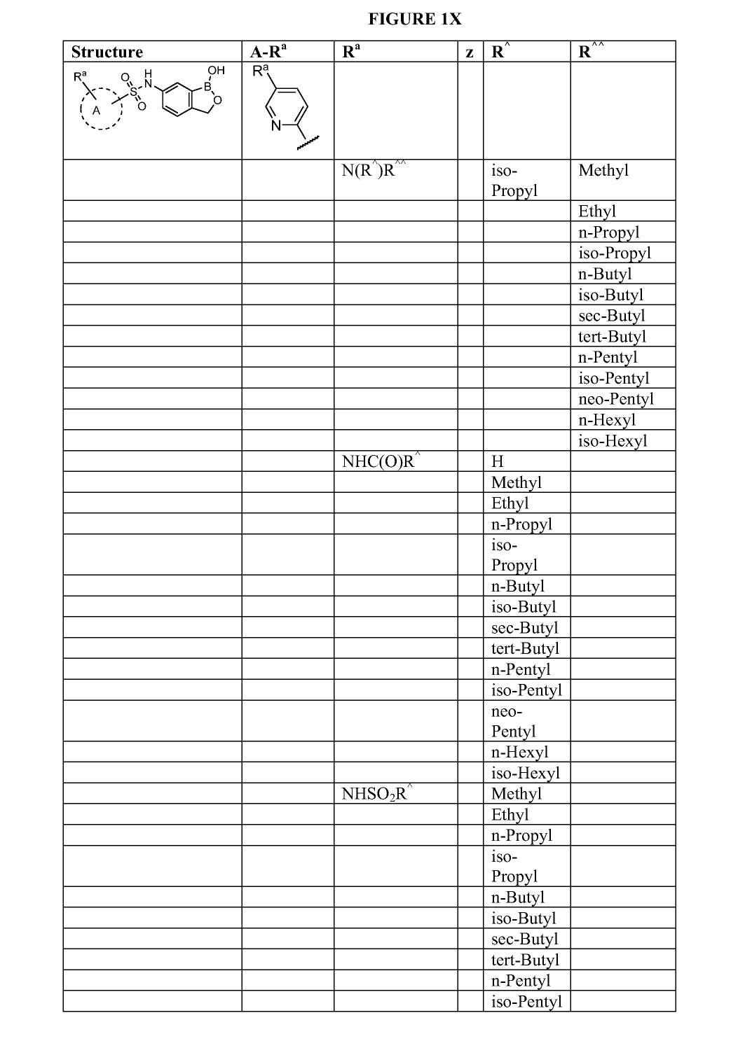

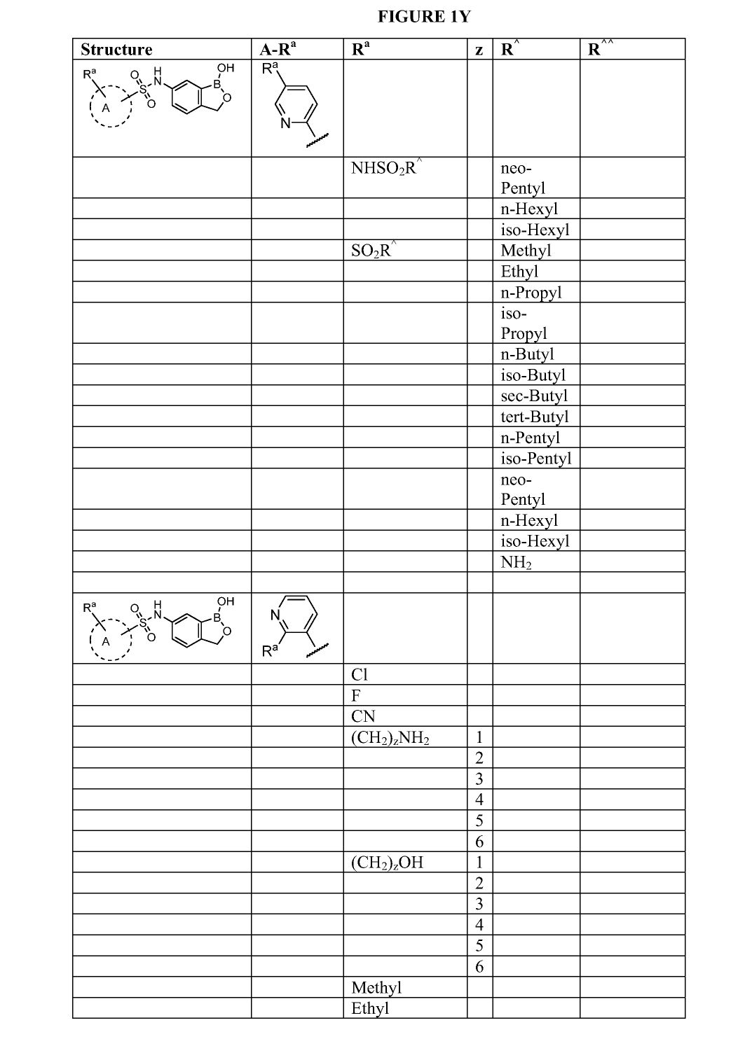

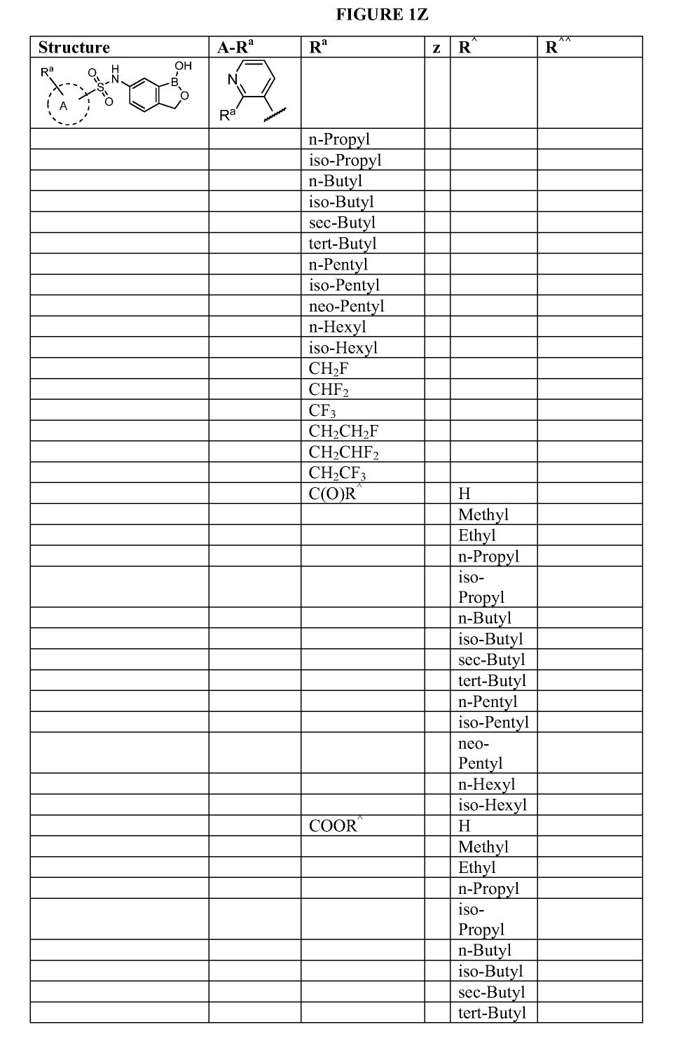

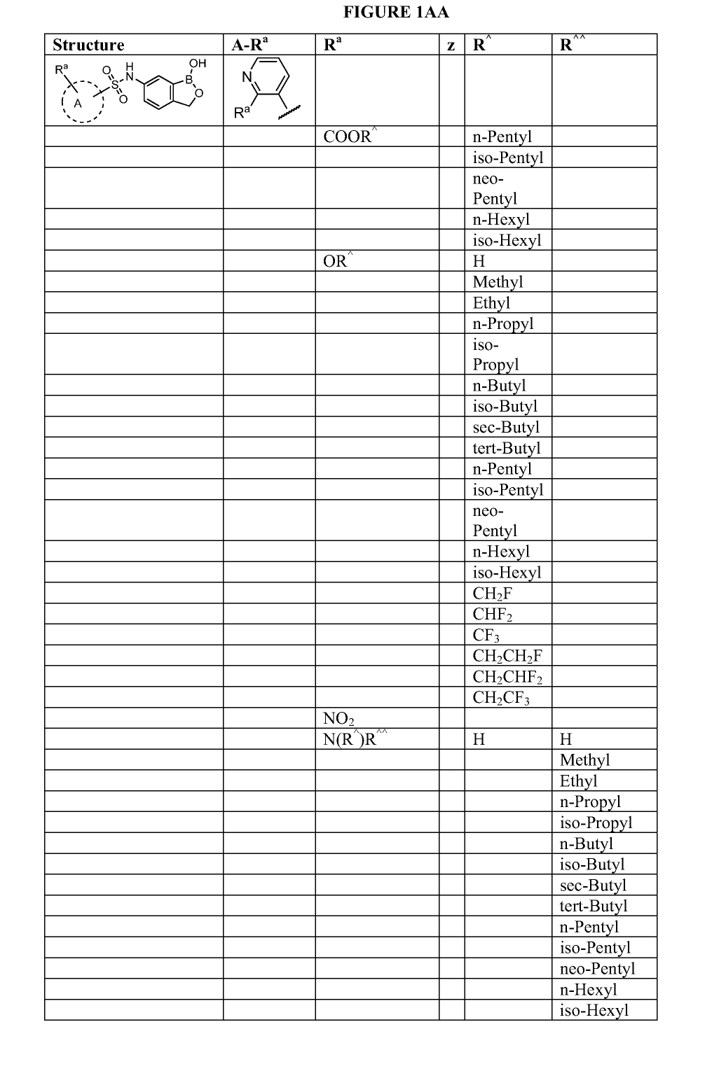

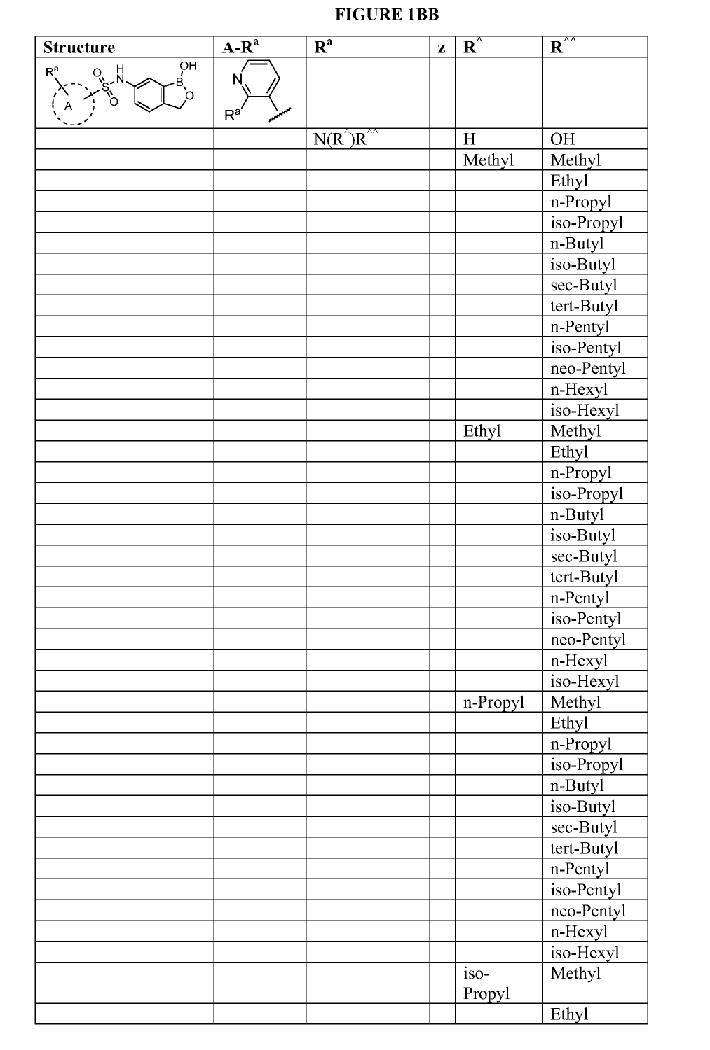

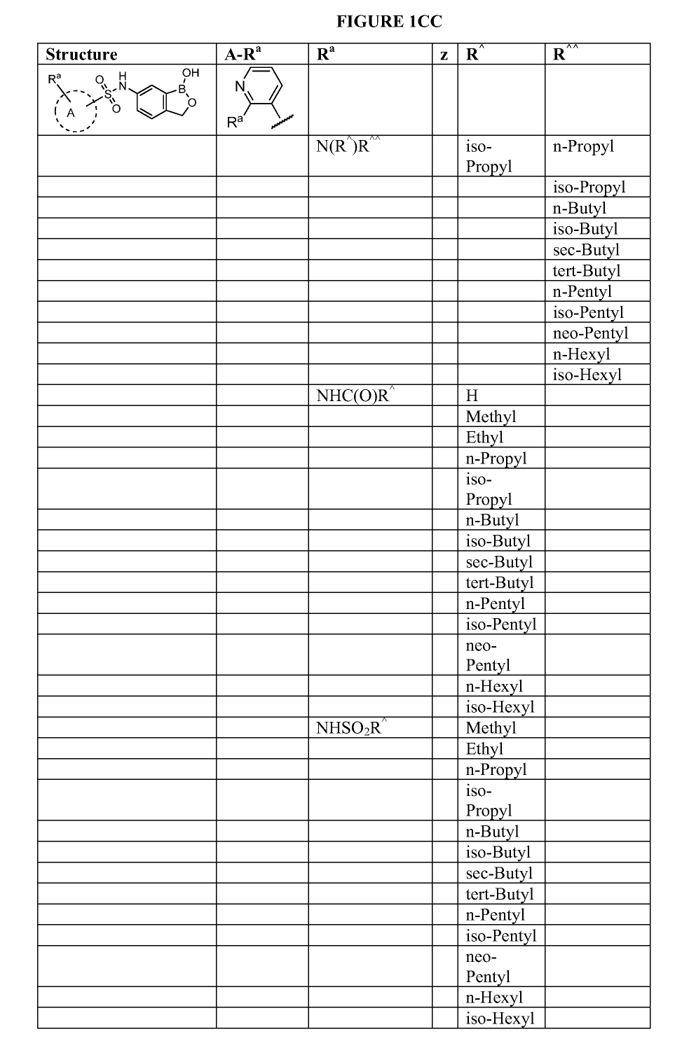

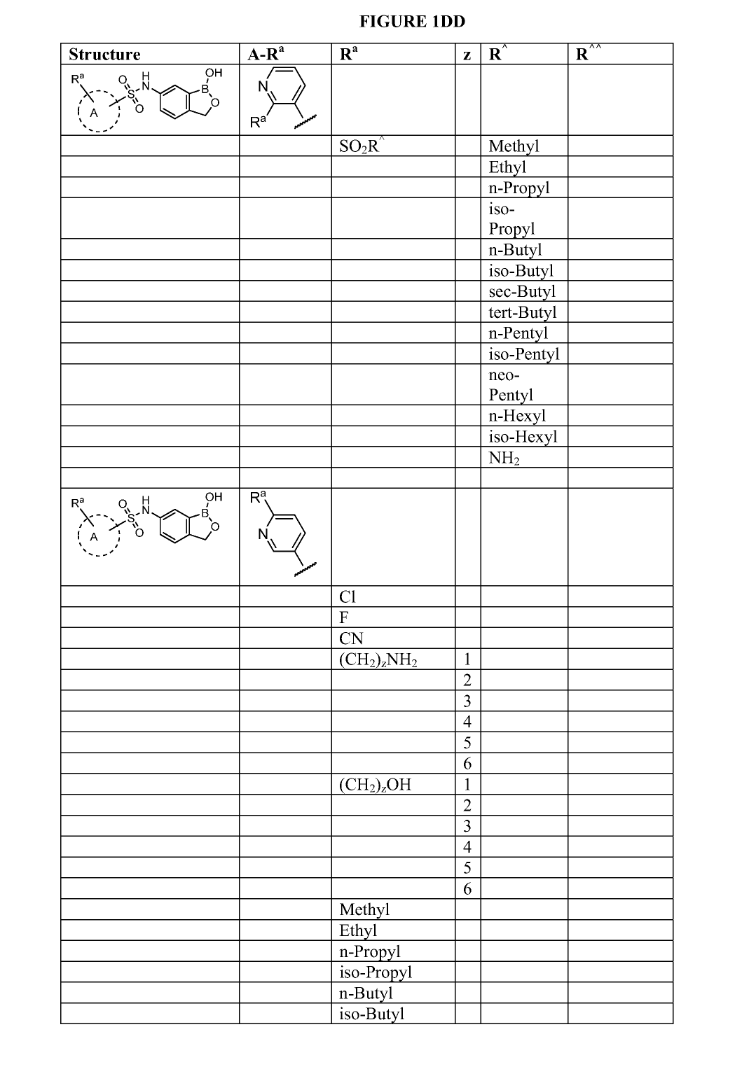

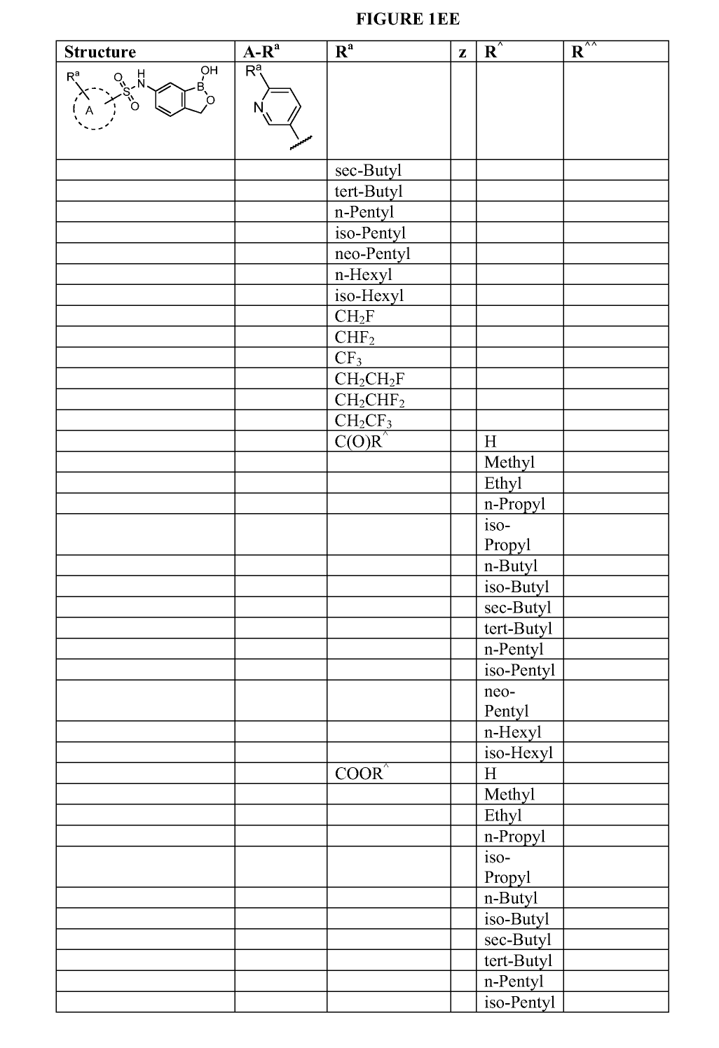

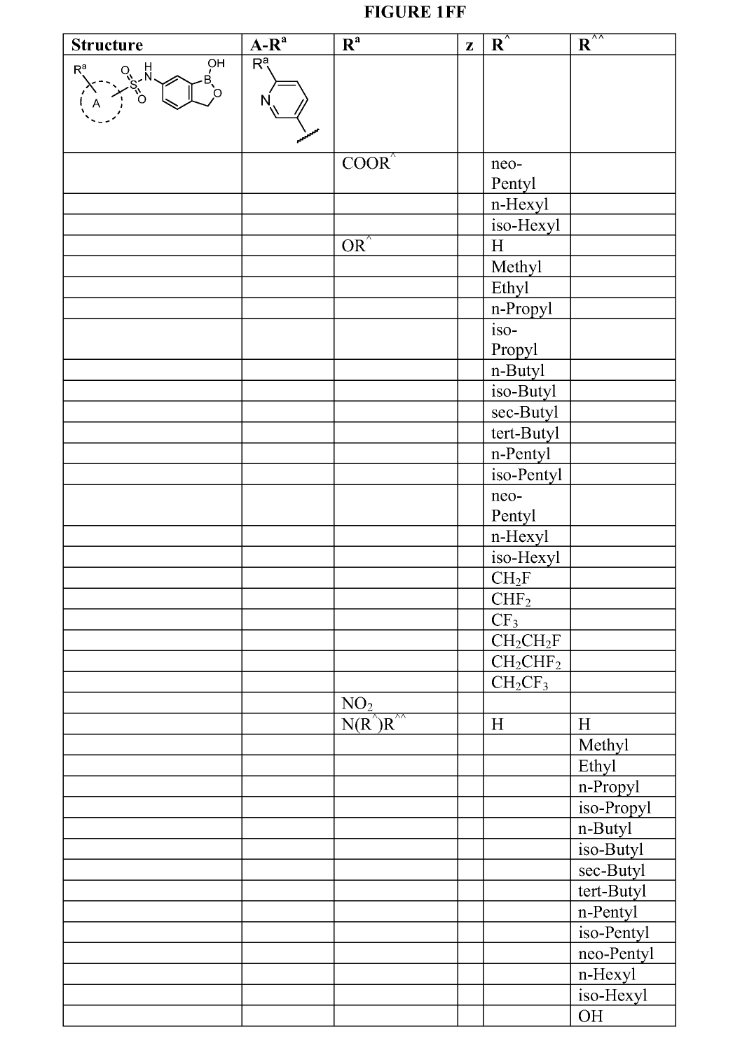

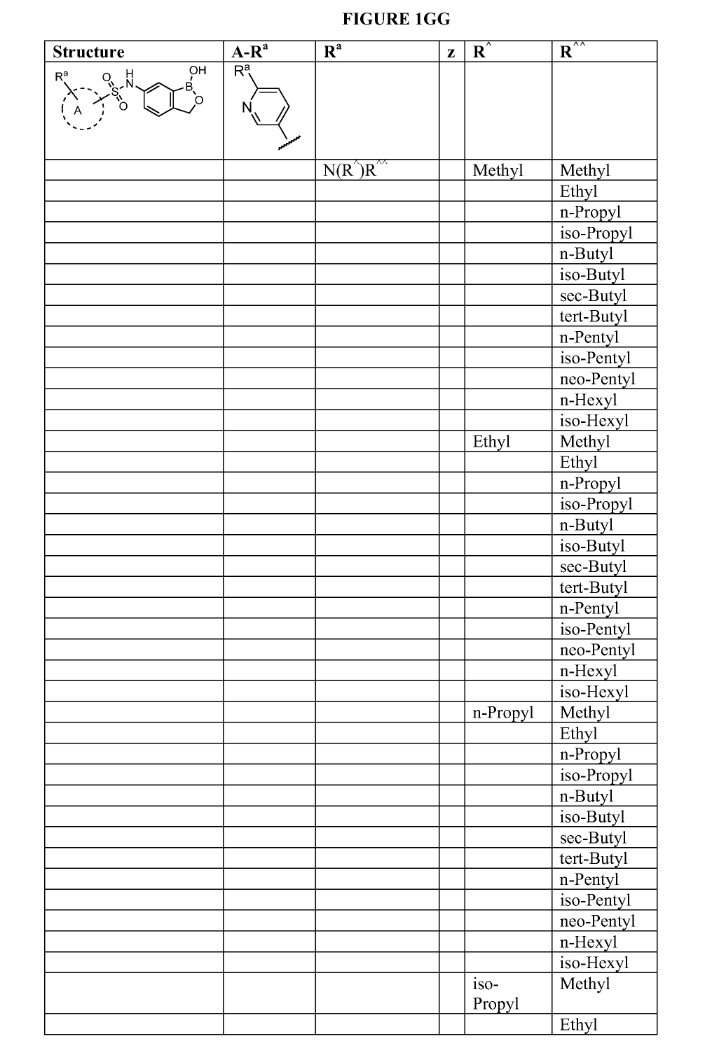

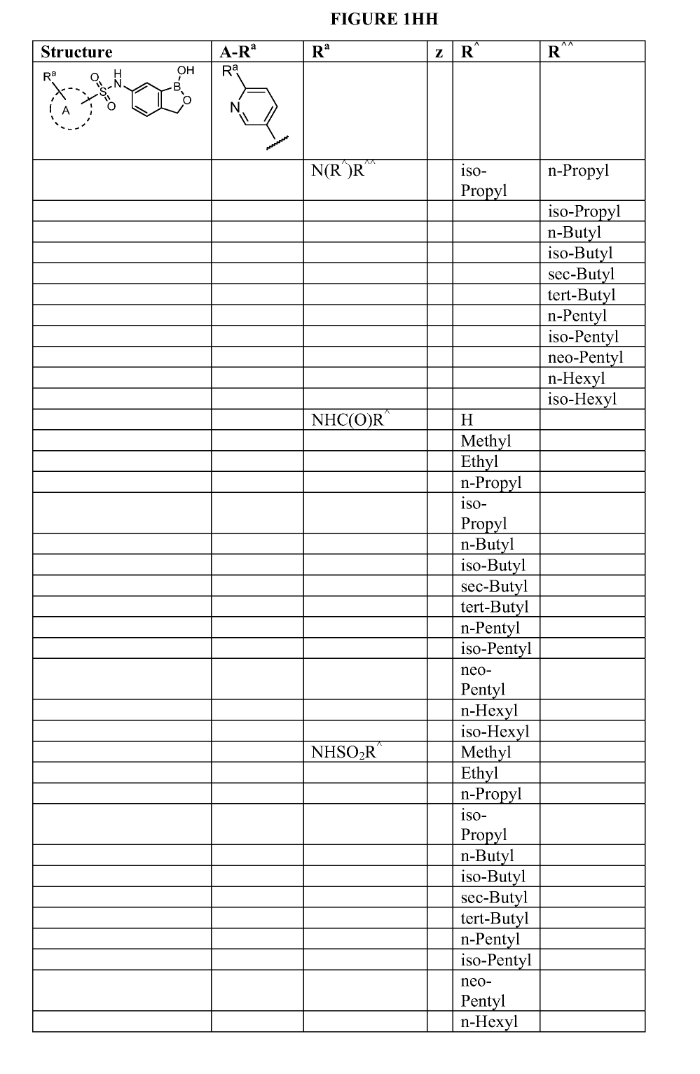

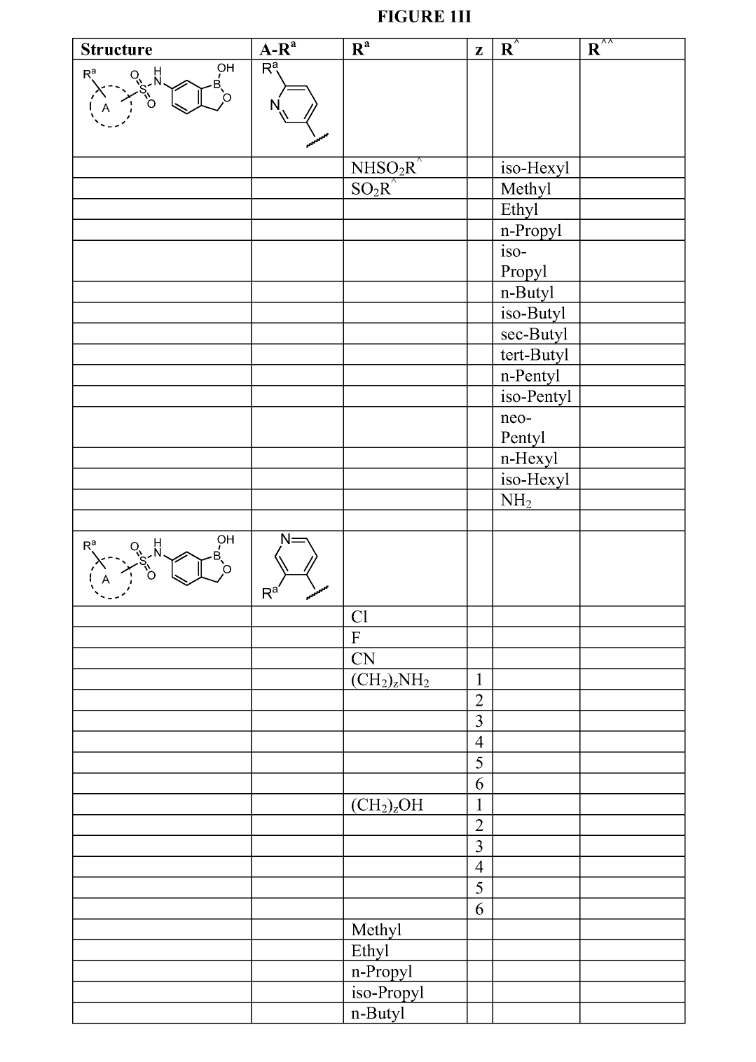

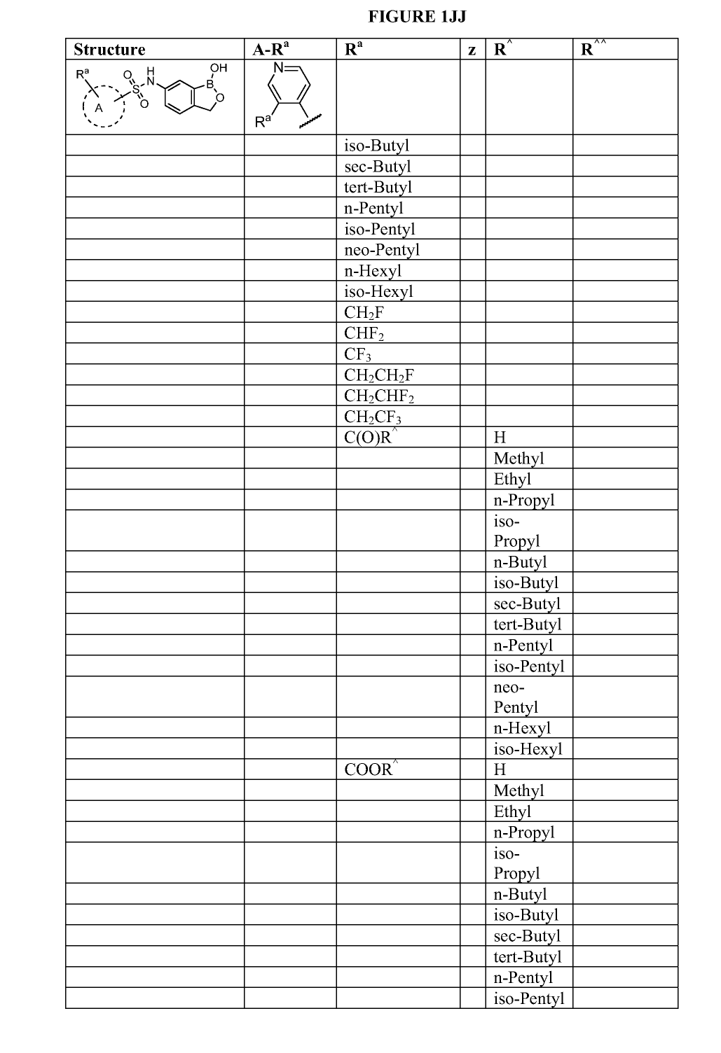

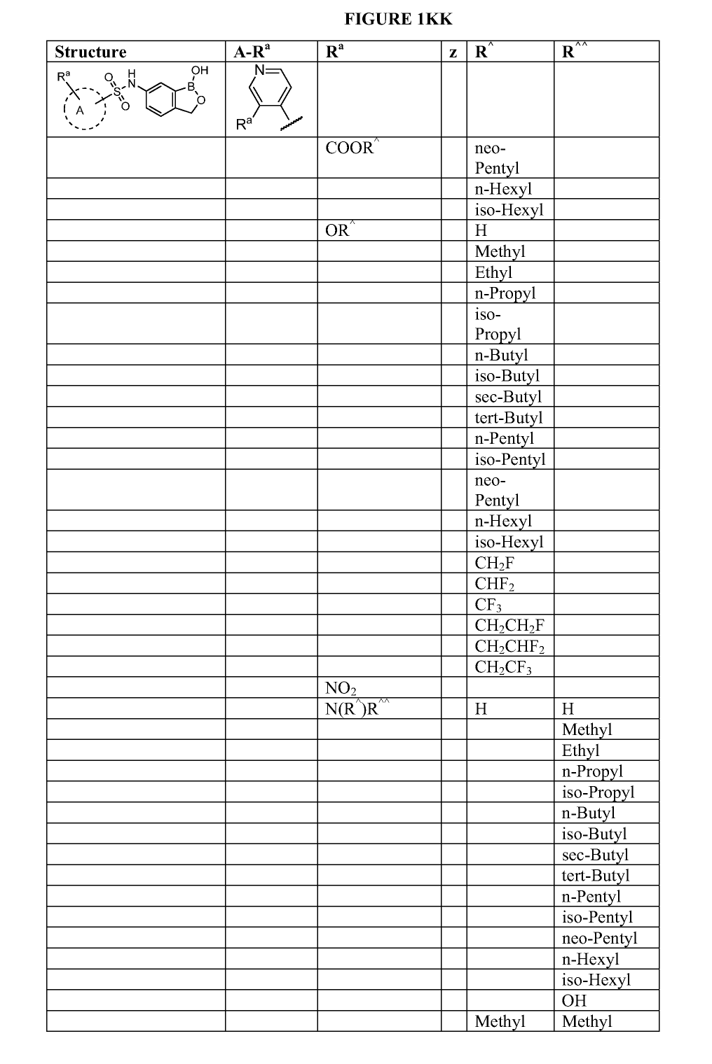

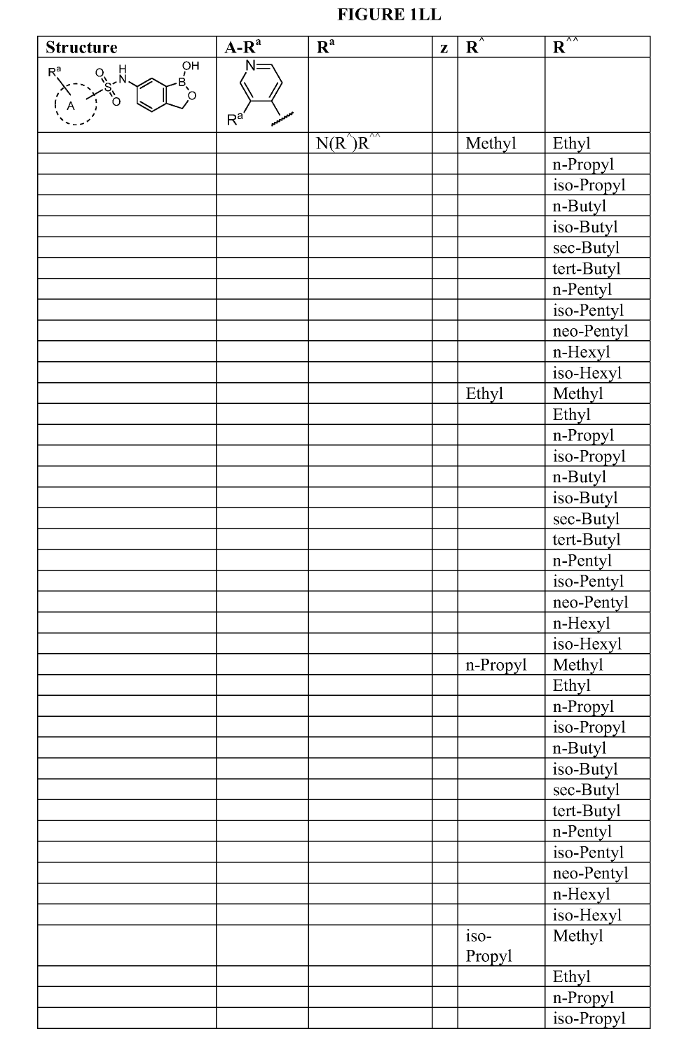

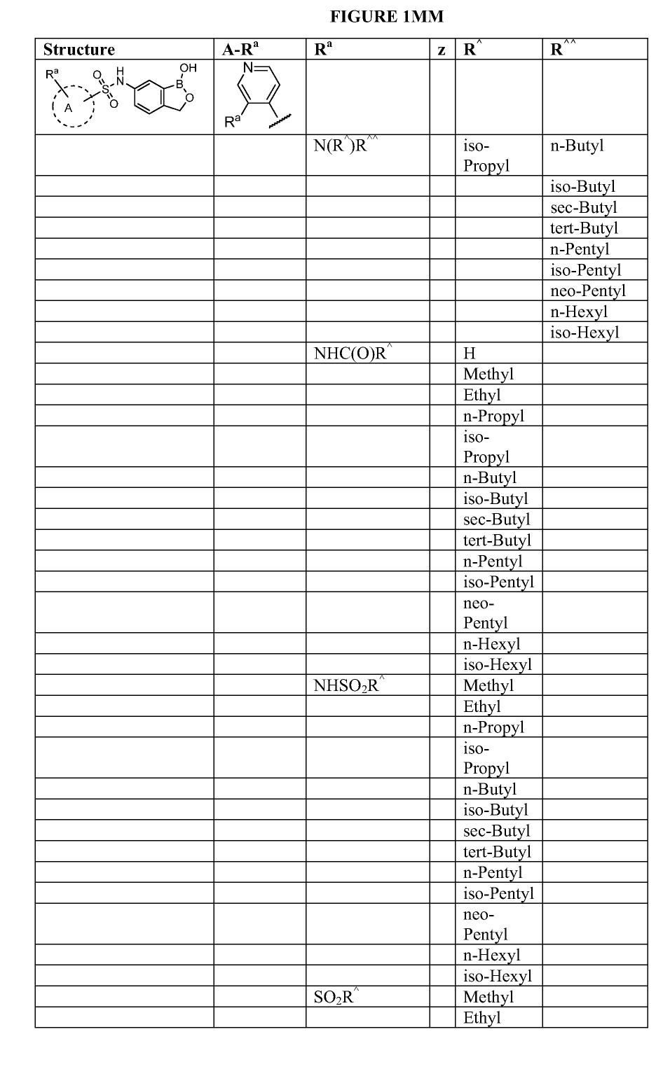

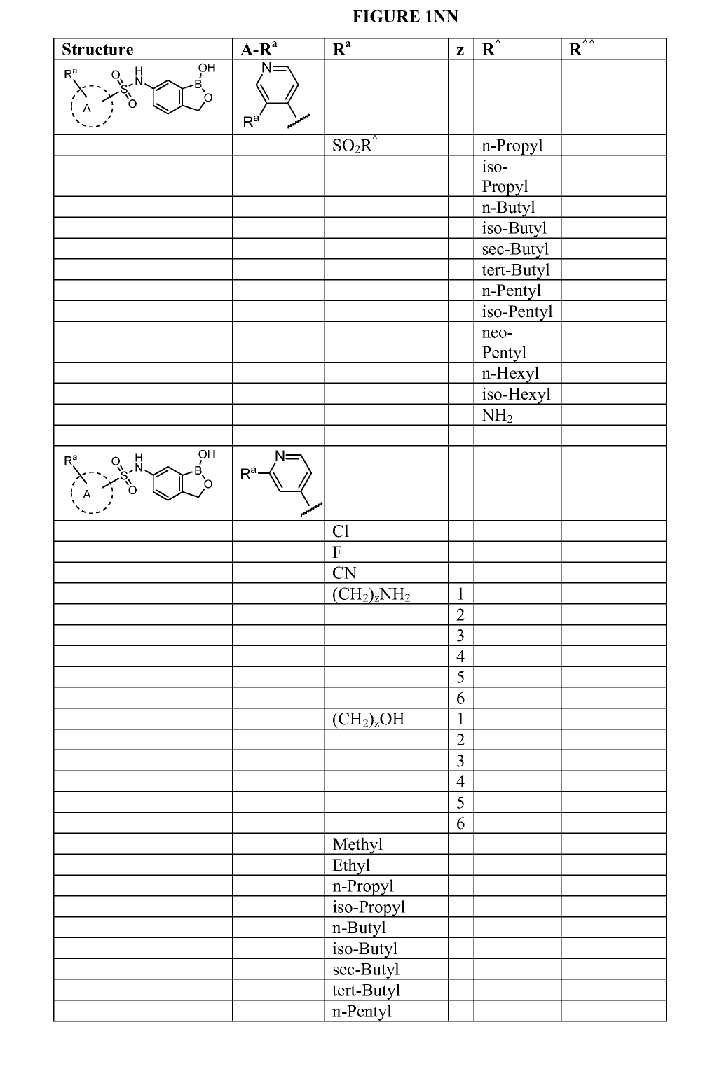

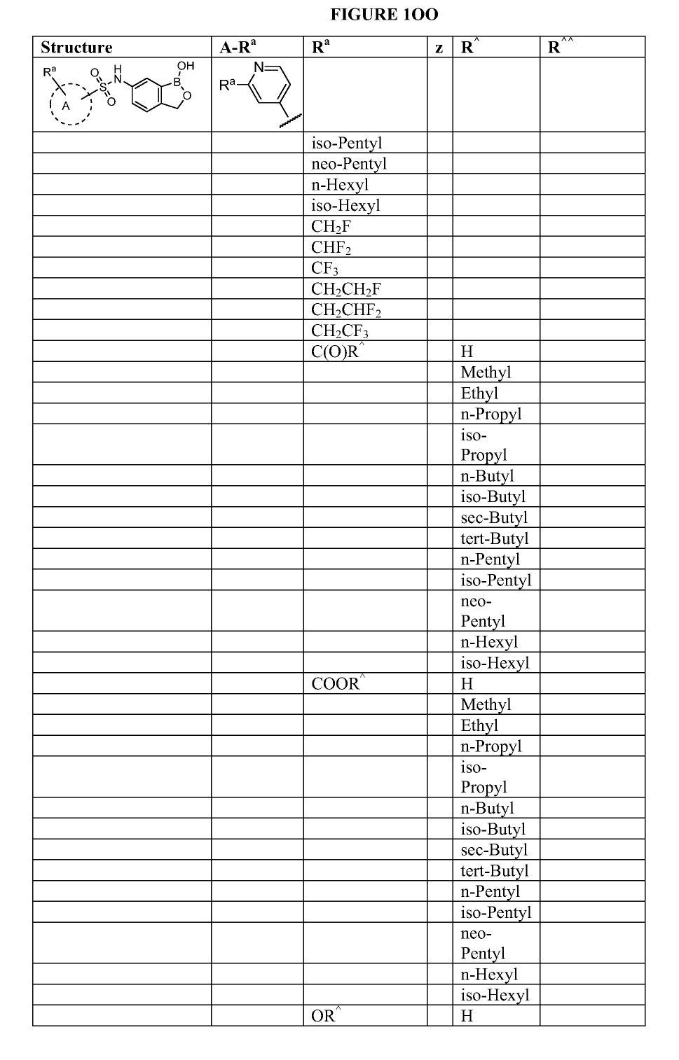

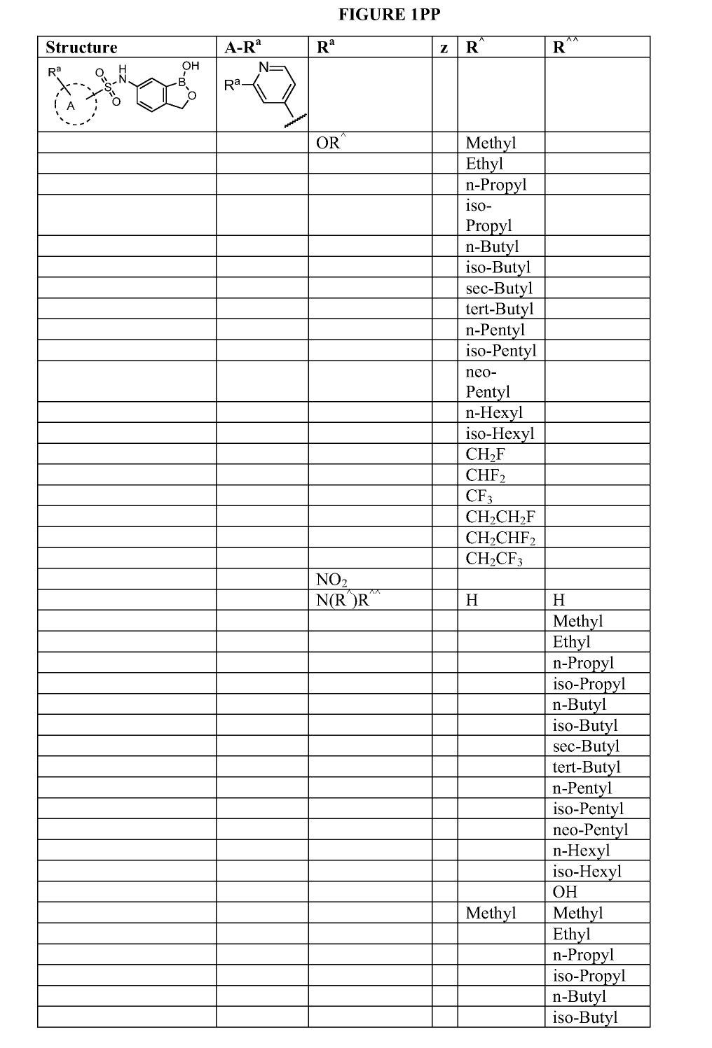

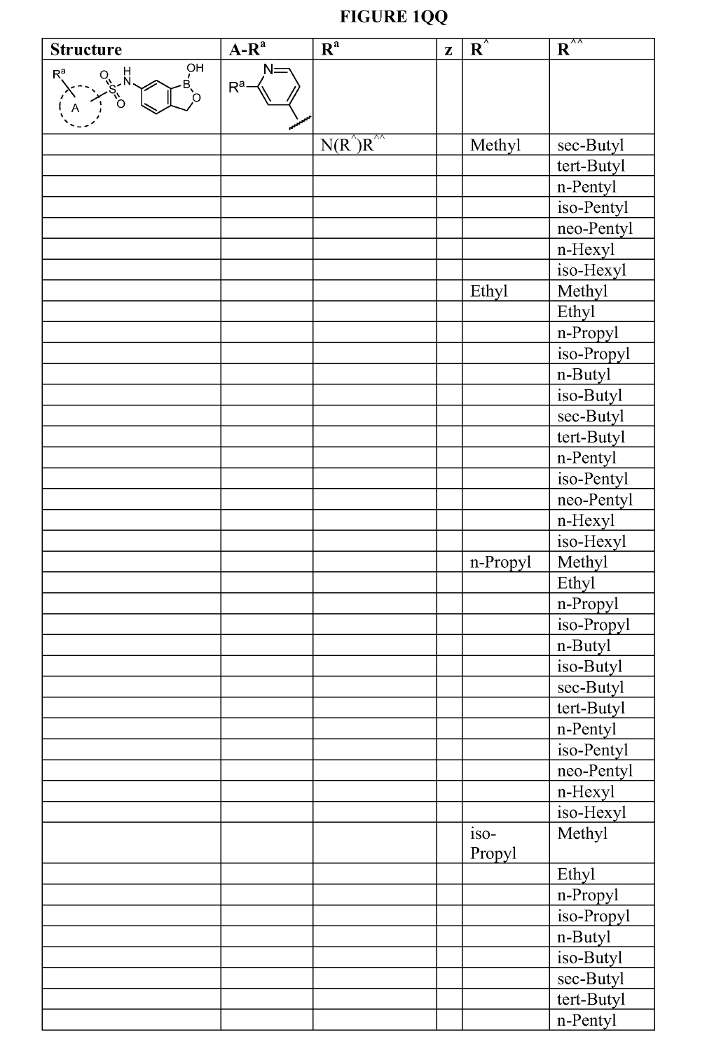

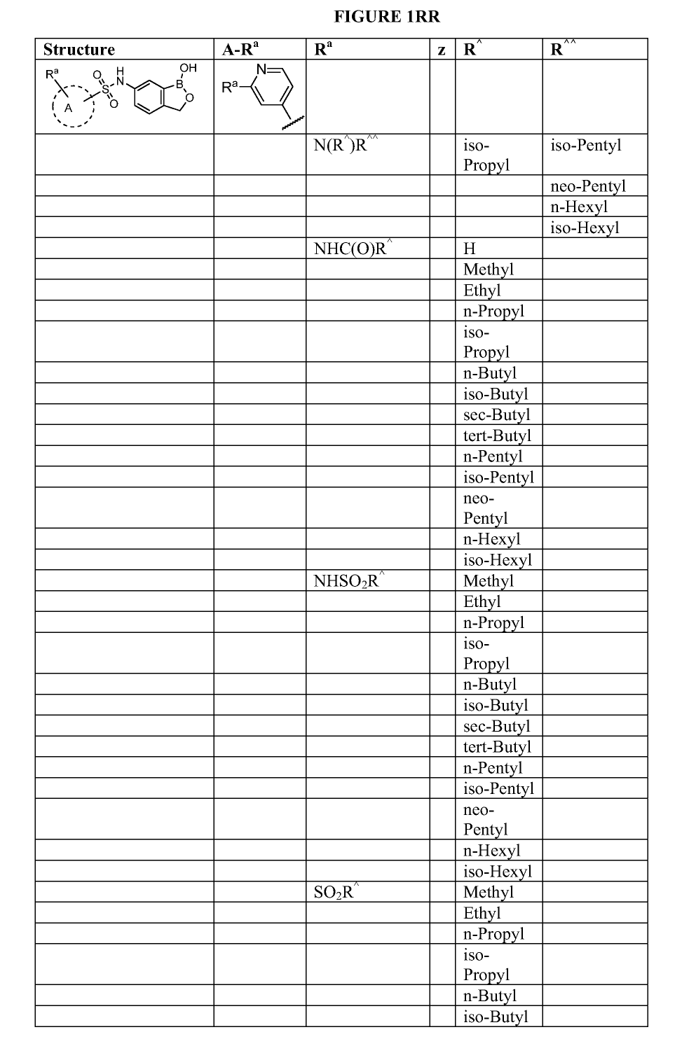

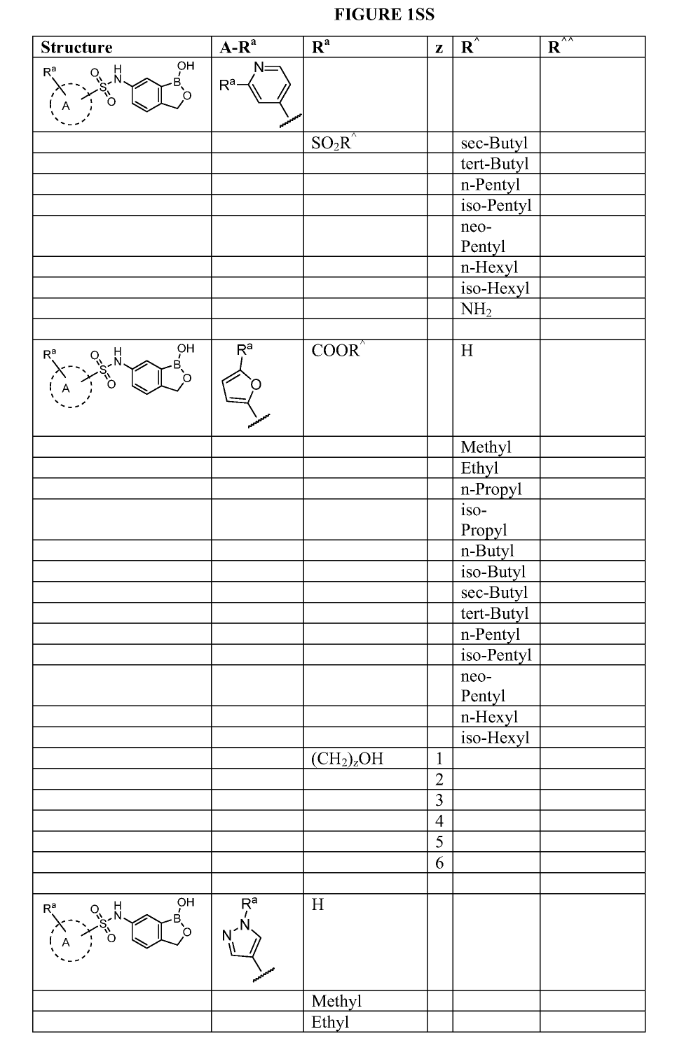

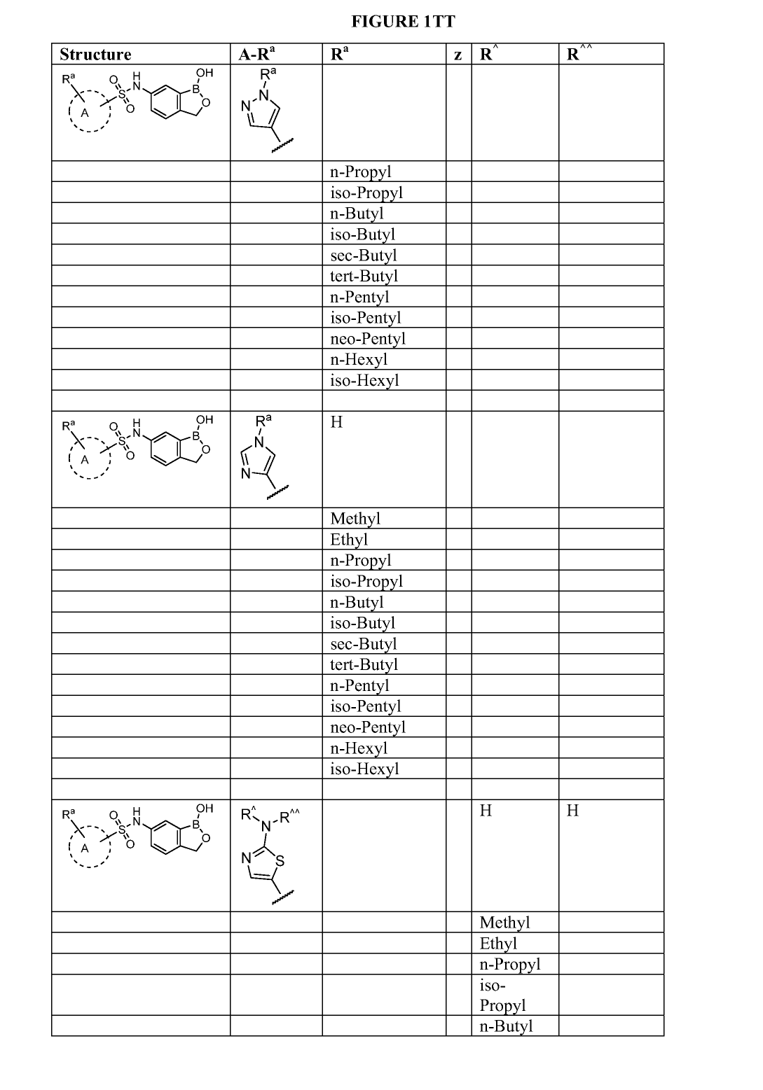

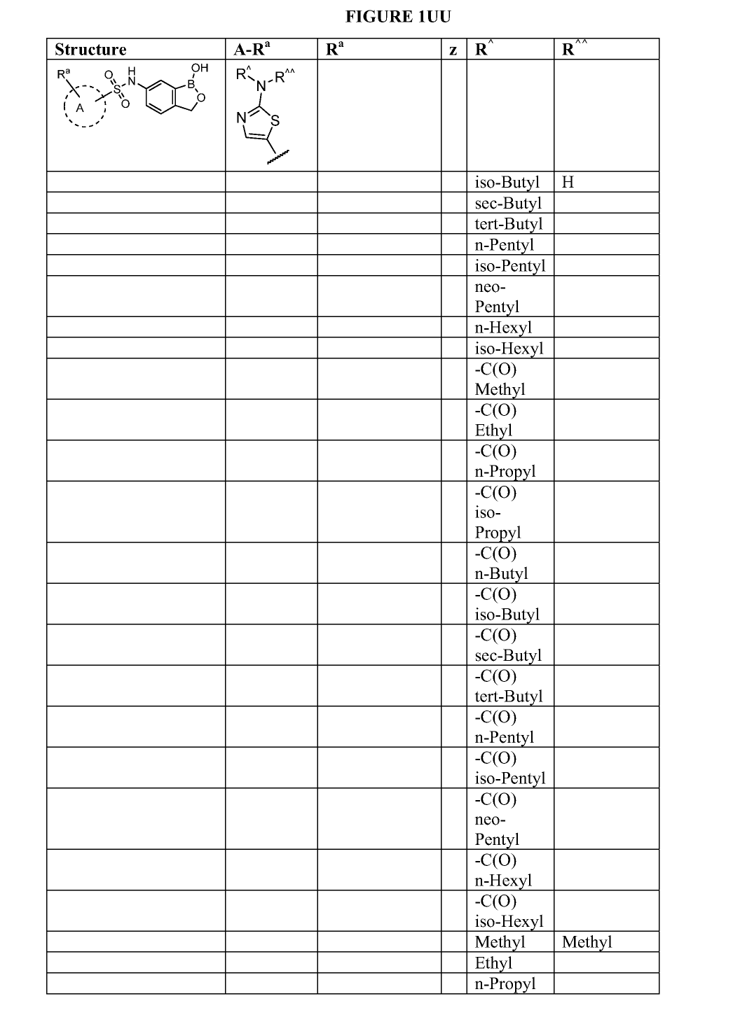

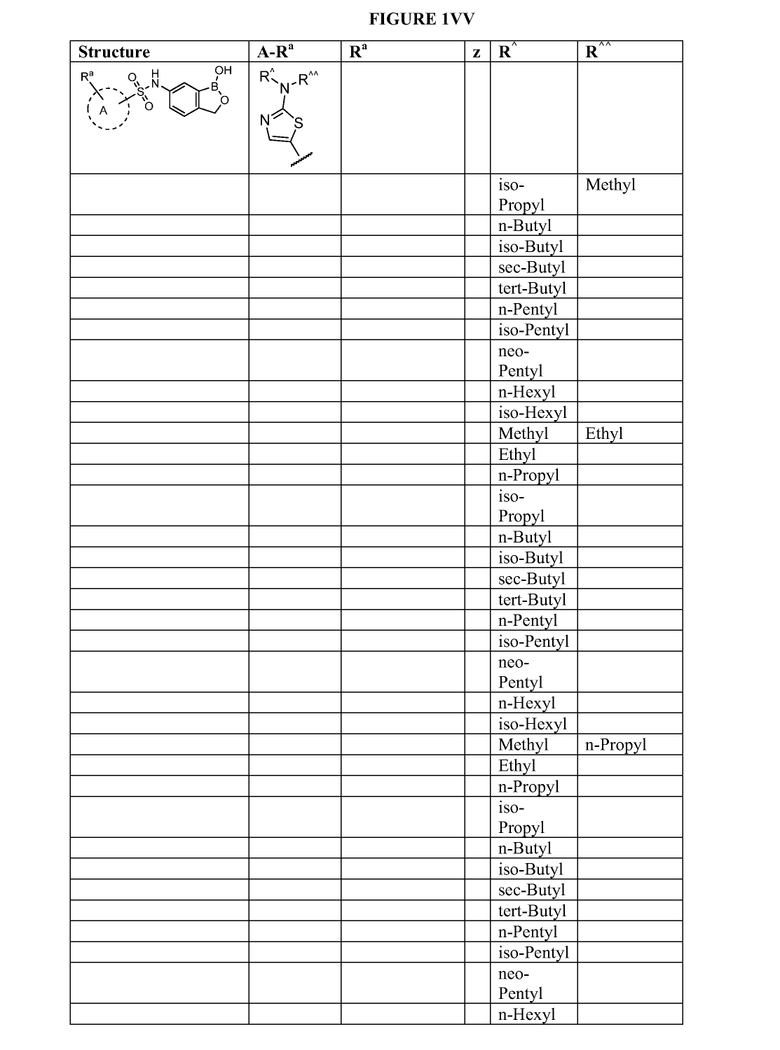

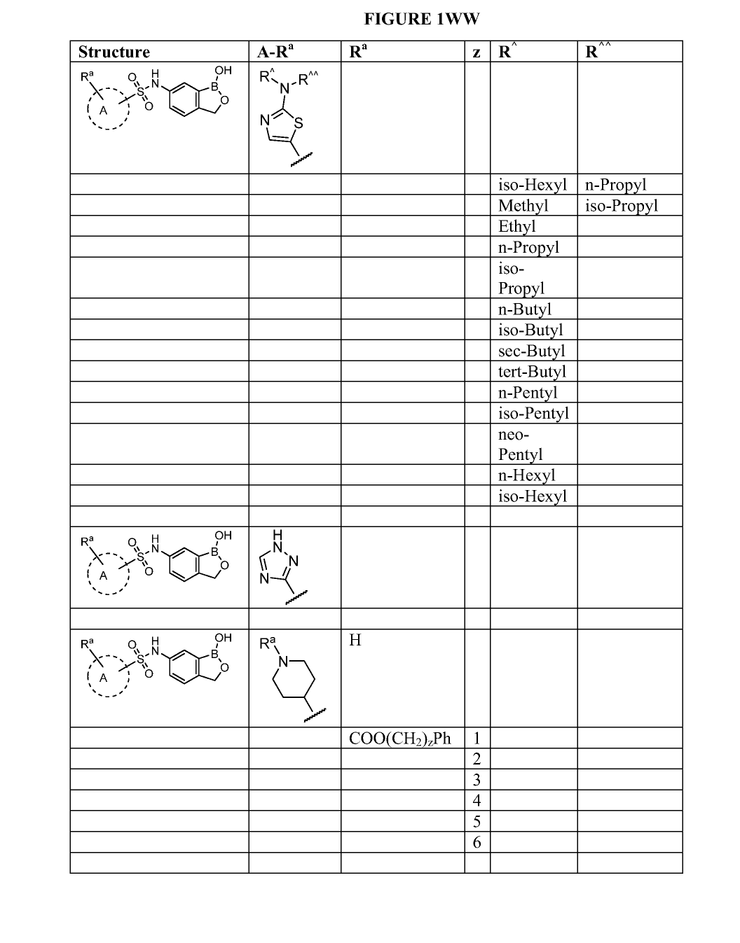

In an exemplary embodiment, the invention provides a compound described herein, or a salt, hydrate or solvate thereof, or a combination thereof. In an exemplary embodiment, the invention provides a compound described herein, or a salt, hydrate or solvate thereof. In an exemplary embodiment, the invention provides a compound described herein, or a salt thereof. In an exemplary embodiment, the salt is a pharmaceutically acceptable salt. In an exemplary embodiment, the invention provides a compound described herein, or a hydrate thereof. In an exemplary embodiment, the invention provides a compound described herein, or a solvate thereof. In an exemplary embodiment, the invention provides a compound described herein, or a prodrug thereof. In an exemplary embodiment, the invention provides a salt of a compound described herein. In an exemplary embodiment, the invention provides a pharmaceutically acceptable salt of a compound described herein. In an exemplary embodiment, the invention provides a hydrate of a compound described herein. In an exemplary embodiment, the invention provides a solvate of a compound described herein. In an exemplary embodiment, the invention provides a prodrug of a compound described herein. In an exemplary embodiment, the invention provides a compound as described in FIG. 1, or a salt thereof. In an exemplary embodiment, the invention provides a compound as described in FIG. 1, or a pharmaceutically acceptable salt thereof.

In an exemplary embodiment, alkyl is a member selected from linear alkyl and branched alkyl. In another exemplary embodiment, heteroalkyl is a member selected from linear heteroalkyl and branched heteroalkyl.

### III. b) Compositions Involving Stereoisomers

As used herein, the term “chiral”, “enantiomerically enriched” or “diastereomerically enriched” refers to a composition having an enantiomeric excess (ee) or a diastereomeric excess (de) of greater than about 50%, preferably greater than about 70% and more preferably greater than about 90%. In general, higher than about 90% enantiomeric or diastereomeric excess is particularly preferred, e.g., those compositions with greater than about 95%, greater than about 97% and greater than about 99% ee or de.

The terms “enantiomeric excess” and “diastereomeric excess” are used interchangeably herein. Compounds with a single stereocenter are referred to as being present in “enantiomeric excess”, those with at least two stereocenters are referred to as being present in “diastereomeric excess”.

The term “enantiomeric excess” is well known in the art and is defined as:

$${ee}_{a} = ( \frac{conc . of a - conc . of b}{conc . of a + conc . of b} ) \times 100$$

The term “enantiomeric excess” is related to the older term “optical purity” in that both are measures of the same phenomenon. The value of ee will be a number from 0 to 100, zero being racemic and 100 being enantiomerically pure. A composition which in the past might have been called 98% optically pure is now more precisely characterized by 96% ee. A 90% ee reflects the presence of 95% of one enantiomer and 5% of the other(s) in the material in question.

Hence, in one embodiment, the invention provides a composition including a first stereoisomer and at least one additional stereoisomer of a compound of the invention. The first stereoisomer can be present in a diastereomeric or enantiomeric excess of at least about 80%, or at least about 90%, or at least about 92% or at least about 95%. In another exemplary embodiment, the first stereoisomer is present in a diastereomeric or enantiomeric excess of at least about 96%, at least about 97%, at least about 98%, at least about 99% or at least about 99.5%. In another embodiment, the compound of the invention is enantiomerically or diastereomerically pure (diastereomeric or enantiomeric excess is about 100%). Enantiomeric or diastereomeric excess can be determined relative to exactly one other stereoisomer, or can be determined relative to the sum of at least two other stereoisomers. In an exemplary embodiment, enantiomeric or diastereomeric excess is determined relative to all other detectable stereoisomers, which are present in the mixture. Stereoisomers are detectable if a concentration of such stereoisomer in the analyzed mixture can be determined using common analytical methods, such as chiral HPLC.

As used herein, and unless otherwise indicated, a composition that is “substantially free” of a compound means that the composition contains less than about 20% by weight, or less than about 15% by weight, or less than about 10% by weight, or less than about 5% by weight, or less than about 3% by weight, or less than about 2% by weight, or less than about 1% by weight of the compound.

As used herein, the term “substantially free of the (or its) enantiomer” means that a composition containing a compound of the invention is made up of a significantly greater proportion of one enantiomer than of its optical antipode. In one embodiment of the invention, the term “substantially free of the enantiomer” means that the compound is made up of at least about 90% by weight of the (R) enantiomer and about 10% by weight or less of the (S) stereoisomer. In a more preferred embodiment of the invention, the term “substantially free of the enantiomer” means that the compound is made up of at least about 95% by weight of the (R) enantiomer and about 5% by weight or less of the (S) stereoisomer. In an even more preferred embodiment, the term “substantially free of the enantiomer” means that the compound is made up of at least about 98% by weight of the (R) enantiomer and about 2% or less of the (S) stereoisomer. In an even more preferred embodiment, the term “substantially free of the enantiomer” means that the compound is made up of at least about 99% by weight of the (R) enantiomer and about 1% or less of the (S) stereoisomer.

In an exemplary embodiment, the invention provides a composition comprising a) a first stereoisomer of a compound described herein, wherein R³ is not H; b) at least one additional stereoisomer of the first stereoisomer, wherein the first stereoisomer is present in an enantiomeric excess of at least 80% relative to said at least one additional stereoisomer. In an exemplary embodiment, the enantiomeric excess is at least 92%. In an exemplary embodiment, the C* stereocenter of the first stereoisomer is in a (R) configuration. In an exemplary embodiment, the C* stereocenter of the first stereoisomer is in a (R) configuration, and R³ is —(CH₂)ₘCOOR²⁰. In an exemplary embodiment, the C* stereocenter of the first stereoisomer is in a (R) configuration, and R³ is —(CH₂)ₘCOOH. In an exemplary embodiment, the C* stereocenter of the first stereoisomer is in a (R) configuration, and R³ is —CH₂COOR²⁰. In an exemplary embodiment, the C* stereocenter of the first stereoisomer is in a (R) configuration, and R³ is —CH₂COOH.

In an exemplary embodiment, the invention provides a composition comprising a compound of the invention, wherein R³ is not H and the C* stereocenter is in a (R) configuration, and said composition is substantially free of the enantiomer of the compound. In an exemplary embodiment, the composition comprises E38, E50 or combinations thereof, wherein the composition is substantially free of the enantiomer of E38 or E50. In an exemplary embodiment, the invention provides a composition comprising a compound described herein, wherein R³ is not H and the C* stereocenter is in a (S) configuration.

### III. c) Combinations Comprising Additional Therapeutic Agents

The compounds of the invention may also be used in combination with additional therapeutic agents. The invention thus provides, in a further aspect, a combination comprising a compound of the invention together with at least one additional therapeutic agent, or a salt, prodrug, hydrate or solvate thereof. In an exemplary embodiment, the compound of the invention is a compound described herein, or a salt thereof. In an exemplary embodiment, the additional therapeutic agent is a compound of the invention. In an exemplary embodiment, the additional therapeutic agent includes a boron atom. In an exemplary embodiment, the additional therapeutic agent does not contain a boron atom. In an exemplary embodiment, the additional therapeutic agent is a compound described in sections III a)-b).

When a compound of the invention is used in combination with a second therapeutic agent active against the same disease state, the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

In another aspect, the invention provides a combination which includes a compound of the invention; and an antibiotic. In an exemplary embodiment, the compound is described herein, or is a pharmaceutically acceptable salt thereof. In an exemplary embodiment, the antibiotic comprises a β-lactam moiety. In an exemplary embodiment, the antibiotic is described herein. In an exemplary embodiment, the antibiotic is a member selected from penicillin G, amoxicillin, ampicillin, azlocillin, carbenicillin, cloxacillin, dicloxacillin, flucloxacillin, mezlocillin, nafcillin, pipericillin, ticarcillin, ceftazidime, cephalothin, cefotaxime, cefpirome, cefepime, and cefoxitin. In an exemplary embodiment, the antibiotic is a member selected from tazobactam, sulbactam and clavulanic acid. In an exemplary embodiment, the antibiotic is a member selected from Ceftazidime, Cephalothin, Cefotaxime, Cefpirome or Cefepime, Cefoxitin, Penicillin G, Amoxicillin, Ampicillin, Azlocillin, Carbenicillin, Cloxacillin, Dicloxacillin, Flucloxacillin, Mezlocillin, Nafcillin, Pipericillin, Ticarcillin, methicillin and temocillin. In an exemplary embodiment, the antibiotic is a member selected from cefacetrile, cefadroxil, cefalexin, cefaloglycin, cefaloridine, cefalotin, cefapirin, cefatrizine, cefazedone, cefazolin, cefradine, cefroxadine, ceftezole, cephalothin and cefazolin. In an exemplary embodiment, the antibiotic is a member selected from cefmetazole, cefonicid, ceforanide, cefotian, cefprozil, cefotetan, cefaclor, cefuroxime, cefamandole and cefoxitin. In an exemplary embodiment, the antibiotic is a member selected from cefdinir, cefditoren, cefetamet, cefixime, cefmenoxime, cefodizime, cefoperazone, cefotzime, cefpiramide, cefsulodin, ceftazidime, ceftibuten, ceftioxime, ceftriaxone, latamoxef, ceftriaxone, cefotaxime and cefpodoxime. In an exemplary embodiment, the antibiotic is a member selected from cefquinome, cefepime and cefpirome. In an exemplary embodiment, the antibiotic is a member selected from ceftobiprole. In an exemplary embodiment, the antibiotic is a member selected from thienamycin, doripenem, panipenem (betamipron), imipenem, meropenem, ertapenem and faropenem. In an exemplary embodiment, the antibiotic is a member selected from benzathine penicillin, benzylpenicillin (penicillin G), phenoxymethylpenicillin (penicillin V) and procaine penicillin.

In an exemplary embodiment, the combination of the invention is a boron-containing compound described herein or a salt thereof, and cefepime. In an exemplary embodiment, the combination of the invention is a boron-containing compound described herein or a salt thereof, and cefepime.

In an exemplary embodiment, the combination of the invention is a boron-containing compound described herein or a salt thereof, and imipenem. In an exemplary embodiment, the combination of the invention is a boron-containing compound described herein or a salt thereof, and imipenem.

In an exemplary embodiment, the combination of the invention is a boron-containing compound described herein or a salt thereof, and meropenem. In an exemplary embodiment, the combination of the invention is a boron-containing compound described herein or a salt thereof, and meropenem.

The individual components of such combinations may be administered either simultaneously or sequentially in a unit dosage form. The unit dosage form may be a single or multiple unit dosage forms. In an exemplary embodiment, the invention provides a combination in a single unit dosage form. An example of a single unit dosage form is a capsule wherein both the compound of the invention and the additional therapeutic agent are contained within the same capsule. In an exemplary embodiment, the invention provides a combination in a two unit dosage form. An example of a two unit dosage form is a first capsule which contains the compound of the invention and a second capsule which contains the additional therapeutic agent. Thus the term ‘single unit’ or ‘two unit’ or ‘multiple unit’ refers to the object which the animal (for example, a human) ingests, not to the interior components of the object. Appropriate doses of known therapeutic agents will be readily appreciated by those skilled in the art.

The combinations referred to herein may conveniently be presented for use in the form of a pharmaceutical formulation. Thus, an exemplary embodiment of the invention is a pharmaceutical formulation comprising a) a compound of the invention; b) an additional therapeutic agent and c) a pharmaceutically acceptable excipient. In an exemplary embodiment, the pharmaceutical formulation is a unit dosage form. In an exemplary embodiment, the pharmaceutical formulation is a single unit dosage form. In an exemplary embodiment, the pharmaceutical formulation is a single unit dosage form which includes a compound of the invention; an antibiotic and a pharmaceutically acceptable excipient. In an exemplary embodiment, the pharmaceutical formulation is a single unit dosage form which includes a compound of the invention; an antibiotic and at least one pharmaceutically acceptable excipient. In an exemplary embodiment, the pharmaceutical formulation is a two unit dosage form. In an exemplary embodiment, the pharmaceutical formulation is a two unit dosage form comprising a first unit dosage form and a second unit dosage form, wherein the first unit dosage form includes a) a compound of the invention and b) a first pharmaceutically acceptable excipient; and the second unit dosage form includes c) an additional therapeutic agent and d) a second pharmaceutically acceptable excipient. In an exemplary embodiment, the pharmaceutical formulation is a two unit dosage form comprising a first unit dosage form and a second unit dosage form, wherein the first unit dosage form includes a) a compound of the invention and b) a first pharmaceutically acceptable excipient; and the second unit dosage form includes c) an antibiotic and d) a second pharmaceutically acceptable excipient.

### III. Additional Compounds of the Invention

Additional compounds of the invention include those formed between the 2′,3′ diol of the ribose ring of a nucleic acid, nucleoside or nucleotide, and a compound of the invention. In an exemplary embodiment, the compound is described herein. In an exemplary embodiment, the compound is a cyclic or acyclic boronic ester such as those described herein. These compounds can be used in an animal to kill or inhibit the growth of a microorganism described herein, as well as to treat the diseases described herein. These compounds can be formed in vitro as well as in vivo. Methods of making these compounds are provided in the Examples section.

In another aspect, the invention provides a compound having a structure according to the following formula:

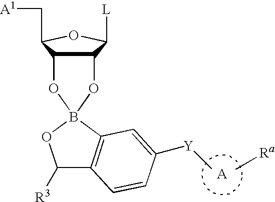

wherein Y, A, R^{a} and R³ are as described herein. L is a member selected from OR⁷, substituted or unsubstituted purine, substituted or unsubstituted pyrimidine, substituted or unsubstituted pyridine and substituted or unsubstituted imidazole. R⁷ is a member selected from H, substituted or unsubstituted alkyl, substituted or unsubstituted heteroalkyl, substituted or unsubstituted cycloalkyl, substituted or unsubstituted aryl and substituted or unsubstituted heteroaryl. A¹ is a member selected from OH, substituted or unsubstituted monophosphate, substituted or unsubstituted diphosphate, substituted or unsubstituted triphosphate,

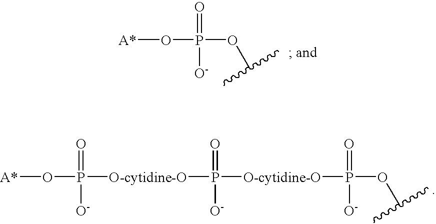

A* is a nucleic acid sequence which comprises between 1 and 100 nucleotides.

In an exemplary embodiment, the compound has the following structure:

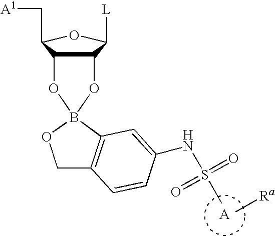

wherein R^{a}, A, L and A¹ are as described herein.

### III.e) Preparation of Boron-Containing Compounds

Compounds of use in the present invention can be prepared using commercially available starting materials, known intermediates, or by using the synthetic methods published in references described and incorporated by reference herein, such as U.S. patent application Ser. No. 12/142,692 and U.S. Pat. Pubs. US20060234981, US20070155699 and US20070293457.

The following general procedures were used as indicated in generating the examples and can be applied, using the knowledge of one of skill in the art, to other appropriate compounds to obtain additional analogues.

### General Procedure 1: Synthesis of Amino 3H-benzo[c][1,2]oxaborol-1-ols

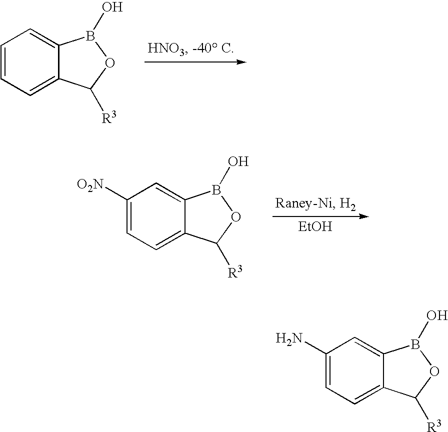

Reference: JACS 1960, 82, 2172. Benzoxaboroles can be mixed with concentrated nitric acid at −40° C. The mixture can be stirred for 30 min then can be added to ice water to precipitate a solid that can be collected by filtration. This crude material can be recrystallized, such as from water, to produce the appropriately substituted 6-nitro 3H-benzo[c][1,2]oxaborol-1-ol. The nitro group can be reduced by dissolving in EtOH and combining with Raney-Ni. This mixture can be subjected to 1.6 atm of hydrogen with agitation in a Parr apparatus for 16 hrs. Nickel catalyst can be removed via filtration and the solvent can be removed under reduced pressure. The resulting residues can be purified by recrystallization from 25% EtOH.

### General Procedure 2: Sulfonylation of Amino 3H-benzo[c][1,2]oxaborol-1-ols

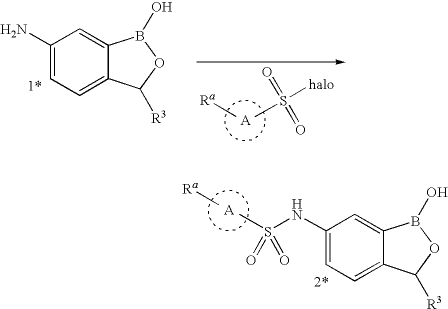

Through subjecting it to sulfonylation conditions, compound 1* can be converted to compound 2*.

In some applications of this general procedure, unsubstituted phenyl or unsubstituted pyridinyl sulfonyl chloride (1-1.2 equiv) and a base (such as NMM, K₂CO₃, or pyridine 3-4 equiv) can be added sequentially to a solution of the amine in MeCN (20 mL/g) at rt. After completion (typical duration O/N) the volatiles can be removed in vacuo. H₂O can be added to the residue and the mixture adjusted to ˜pH 6 with dilute HCl. The aqueous layer can be then extracted with an organic solvent (such as EtOAc), and the combined organic fractions can be dried with a desiccant, such as Na₂SO₄ or MgSO₄, filtered, and concentrated in vacuo. The product can be typically purified by either recrystallization from H₂O, trituration with CH₂Cl₂ or EtOAc, or flash chromatography.

### General Procedure 3: Deprotection of Benzyl Protected Alcohols

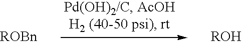

A mixture of the benzylated alcohol (1 equiv) and 20% Pd(OH)₂ on carbon (50% weight-wet, 1:2 w/w substrate to catalyst) in glacial AcOH (10 mL/g) can be shaken under an atmosphere of H₂ (40-50 psi) in a Parr shaker. Once the reaction is complete (TLC), the mixture can be filtered through Celite®. The filtrate can be concentrated in vacuo and the remaining AcOH can be removed by co-evaporation with toluene (3×) to give the alcohol. Further purification can be carried out by flash chromatography or preparative HPLC as required.

### General Procedure 4: Mitsunobu Conditions

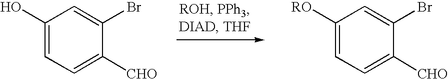

DIAD (1 equiv) can be added to a solution of the phenol (1 equiv) and PPh₃ (1 equiv) in anhydrous THF (200 mL/7 g phenol). The mixture can be stirred at rt until the reaction is complete (as determined by TLC). The mixture can be then concentrated in vacuo. Et₂O can be added to the residue and the mixture can be then concentrated in vacuo. Et₂O can be added again and the precipitate that formed can be removed by filtration. The filtrate can be extracted with 2 N NaOH and H₂O. The organic layer can be dried (Na₂SO₄) and concentrated in vacuo. The residue can be further purified by flash chromatography.

### General Procedure 5

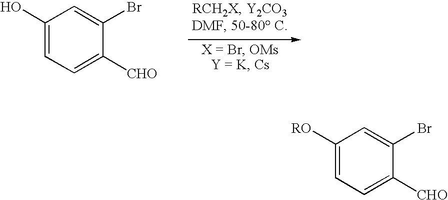

A solution of the alkyl halide or mesylate (1-1.5 equiv), 2-bromo-3-hydroxy-benzaldehyde (1 equiv), and either K₂CO₃ (1-1.2 equiv) or Cs₂CO₃ (1.5-2 equiv), in DMF can be stirred at 50-80° C. (bath temp) until the reaction is complete (typically O/N). The reaction mixture cooled to rt, diluted with H₂O, and extracted with EtOAc. The organic fractions can be washed with H₂O then brine, dried (MgSO₄), and concentrated in vacuo. Further purification can be performed by flash chromatography if required.

### General Procedure 6: Borylation of Aromatic Compound

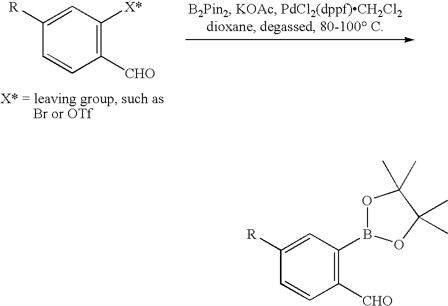

A solution of aryl bromide or triflate in anhydrous 1,4-dioxane or THF (20 mL/1 g) was added B₂pin₂ (2 equiv) and KOAc (3 equiv) at rt, then degassed with N₂ for 10 to 40 min. PdCl₂(dppf).CH₂Cl₂ (4-8 mol %) can be added and the resulting solution can be stirred at 65-100° C. until the reaction was complete (2 to 24 h). The solution can be cooled to rt, filtered through Celite® or silica gel and concentrated in vacuo. The residue can be taken up in EtOAc. The organic layer can be then washed with H₂O then brine, dried (Na₂SO₄), filtered, and concentrated in vacuo. The product can be typically purified by flash chromatography.

### General Procedure 7: Borylation of Phenols via their Aryl Triflates

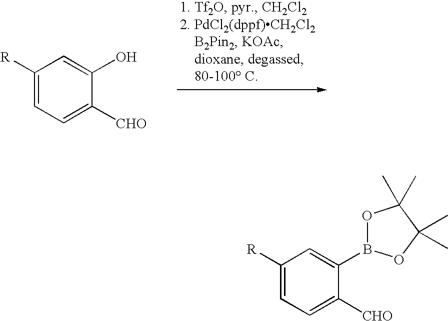

Trifluoromethanesulfonic anhydride (1.2 equiv) can be added dropwise to a solution of pyridine (1.2 equiv) and the phenol in CH₂Cl₂ (40 mL/8.6 g) at 0° C. (bath temp). The reaction mixture can be then allowed to warm to rt and can be stirred until complete consumption of starting material (as determined by TLC). Et₂O and 2 N HCl were then added. The organic layer can be separated and washed with sat. NaHCO₃ then brine. The organic layer can be dried (Na₂SO₄) and filtered through a short silica gel plug, washing with Et₂O. The filtrate can be concentrated in vacuo to give the desired triflate that can be used directly in a subsequent general procedure.

### General Procedure 8: Ring Closure of Substituted 2-(4,4,5,5-Tetramethyl-[1,3,2]dioxaborolan-2-yl)-benzaldehydes

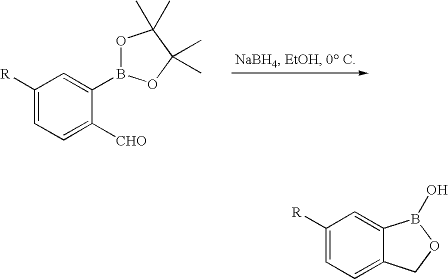

NaBH₄ (1.5 equiv) can be added portionwise to an ice-cold solution of the aldehyde in alcohol (typically absolute EtOH or anhydrous MeOH (c=0.1 M). The reaction can be allowed to warm to rt and monitored by TLC. The mixture can be then acidified to ˜pH 3 using a 1 N NaHSO₄ or 2 M HCl and stirred O/N. The precipitate can be collected by filtration, washed repeatedly with H₂O and dried in vacuo. Further purification can be carried out by flash chromatography when required.

### General Procedure 9: Henry Reaction of Substituted 2-(4,4,5,5-Tetramethyl-[1,3,2]dioxaborolan-2-yl)-benzaldehydes

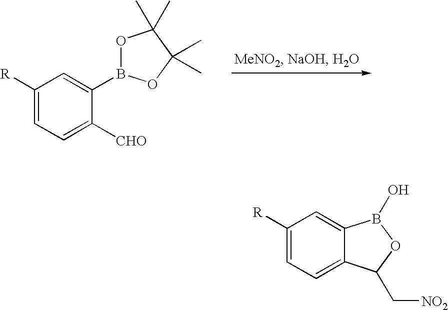

NaOH aq. (1.0 equiv) can be added to the aldehyde (either in H₂O or THF) at rt and the reaction mixture can be stirred at rt for 5 min. MeNO₂ (3 equiv) can be added dropwise and the mixture can be stirred at rt for 16 h. The reaction mixture can be acidified with 2 N HCl and extracted with EtOAc. The organic fraction can be washed with H₂O then brine, dried (MgSO₄), and concentrated in vacuo. Purification can be typically accomplished by either flash chromatography or precipitation from the acidified reaction mixture.

### General Procedure 10: Henry Reaction Using Phase Transfer Catalyst of Substituted 2-(4,4,5,5-Tetramethyl-[1,3,2]dioxaborolan-2-yl)-benzaldehydes

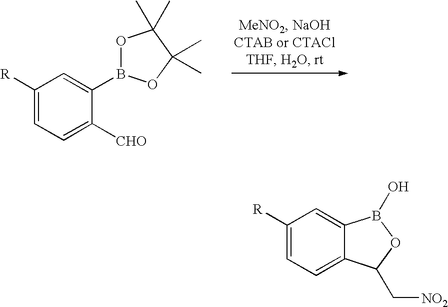

CTAB or CTACl (5 mol %) can be added to a mixture of MeNO₂ and aldehyde, in aq. NaOH, and THF (1 mL/300 mg aldehyde) at rt. The reaction can be monitored by TLC. Upon completion (typically 1-1.5 h), the mixture can be adjusted to pH 2-3 using 2 N HCl or 1 M NaHSO₄ and the mixture can be then stirred for 30 min. The solid can be filtered and dried to afford the desired nitro compound which can be used directly in next step. If there was no precipitation, the organic material can be extracted from the reaction mixture with EtOAc. The organic fraction can be then dried (MgSO₄) and concentrated in vacuo. The residue can be purified by flash chromatography.

### General Procedure 11: Reduction of Alkyl Nitro and/or Alkyl Nitrile Compounds to N-Boc Protected Amines

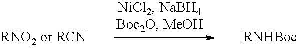

Boc₂O (2 equiv) and NiCl₂.6H₂O (1 equiv) can be added to a stirred solution of the alkyl nitro or alkyl nitrile in anhydrous MeOH (3 mL/mmol) at rt. Stirring can be continued until most of the NiCl₂ had dissolved in MeOH (typically ˜10 min). The reaction mixture can be then cooled to 0° C. (bath temp) and NaBH₄ (6 equiv) was added portionwise over 10 min. The reaction can be exothermic, effervescent, and can result in the formation of a finely divided black precipitate. The reaction mixture can be allowed to warm to rt and left to stir O/N. The mixture can be then concentrated in vacuo and the residue diluted with EtOAc. The resulting suspension can be filtered through Celite and the filtrate concentrated in vacuo. The residue can be then further purified by flash chromatography if required.

### General Procedure 12: Deprotection of Boc-Protected Amines

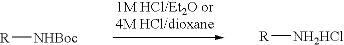

A mixture of the N-Boc protected amine and either 1 M HCl in Et₂O or 4 M HCl in dioxane (2 mL/mmol) can be stirred at rt. After the complete consumption of starting material (monitored by TLC, typically 3-16 h), the mixture can be concentrated in vacuo and the crude residue triturated with Et₂O and filtered. If necessary, the final product was purified by preparative HPLC.

### General Procedure 13: Reduction of Alkyl Nitro and/or Alkyl Nitrile Using Raney Nickel

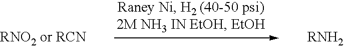

A mixture of the 3-nitromethyl-3H-benzo[c][1,2]oxaborol-1-ol, Raney Ni (2 equiv w/w), 2.0 M NH₃ in EtOH (5 mL/1 g), and absolute EtOH (20 mL/1 g) can be shaken under an atmosphere of H₂ (40-50 psi) for 3 h at rt. The resultant mixture can be filtered through a pad of Celite and washed with EtOH. The filtrate can be concentrated in vacuo to give the free amine.

### General Procedure 14: Reduction of Substituted-3-nitromethyl-3H-benzo[c][1,2]oxaborol-1-ols Using Pearlman's Catalyst

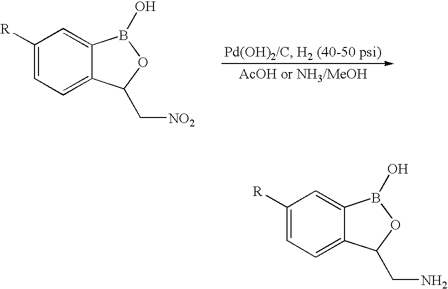

A mixture of the 3-nitromethyl-3H-benzo[c][1,2]oxaborol-1-ol (1 equiv) and 20% Pd(OH)₂ on carbon (50% weight-wet, 1:2 w/w substrate to catalyst) in glacial AcOH (10 mL/g) or 2 M NH₃ in MeOH can be shaken under an atmosphere of H₂ (45-50 psi) in a Parr shaker. Once the reaction is complete (TLC), the mixture can be filtered through Celite®. The filtrate can be concentrated in vacuo to give a gummy material. Remaining AcOH can be removed by co-evaporation with toluene (3×) to give the amine, typically as a fluffy solid. Alternatively remaining ammonia can be removed by diluting with an appropriate solvent like methanol or ether followed by concentration in vacuo. Purification can be typically accomplished by preparative HPLC.

### General Procedure for Chiral HPLC Separation of Enantiomers

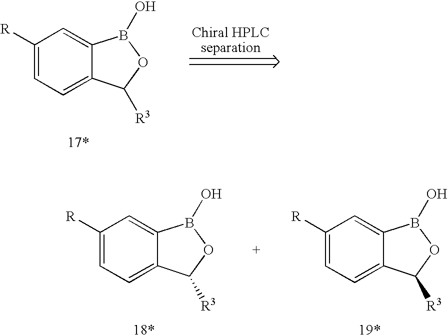

Through subjecting it to chiral HPLC separation conditions, compound 17* can be separated into enantiomers 18* and 19*.

The separation of the two enantiomers can be achieved by dissolving the material in a suitable solvent and applying to an appropriate chiral column and eluent system. The collected separated enantiomer samples can be then concentrated and used in the next step without further purification. Using this technique, it is possible to achieve a range of enantiomeric excesses of the separated enantiomers.

### General Procedure for Chiral Synthesis of 6-R-substituted-3-aminomethylbenzoxaboroles

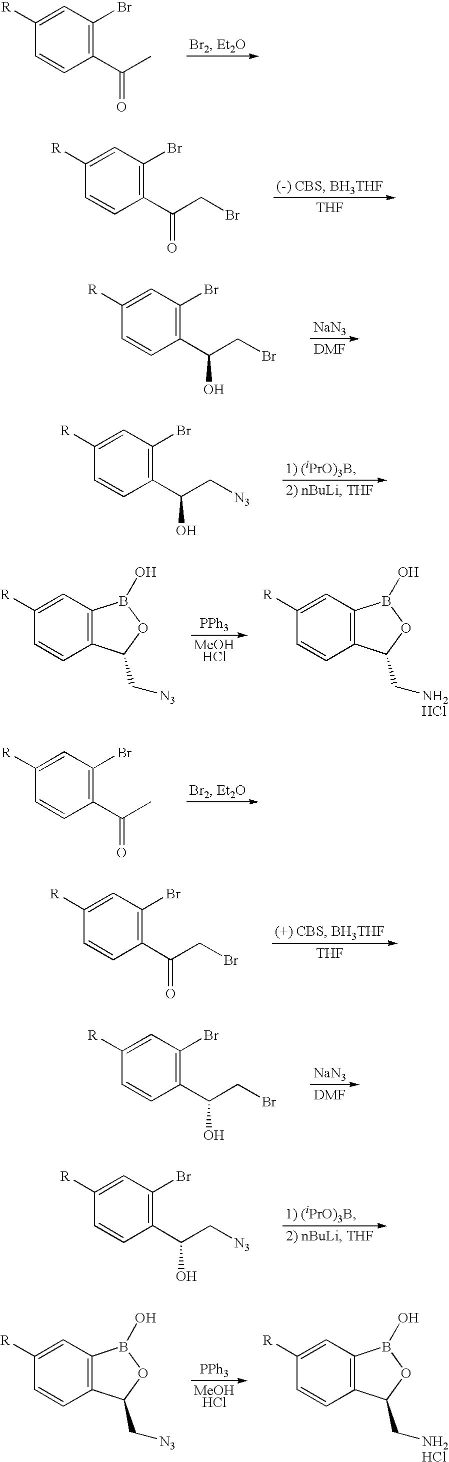

The direct stereospecific synthesis of 6-R-substituted 3-aminomethylbenzoxaboroles can be achieved starting from the 5- or 6-substituted 2-bromoacetophenone. Bromine (1.0 eq) is added slowly to appropriately substituted 2′-bromoacetophenone (1.0 eq) in diethyl ether at room temperature and stirred for 2 hours. Water is added and the reaction mixture stirred until the color fades. The phases are separated and the aqueous layer extracted with diethyl ether. The combined organic phases are washed with brine, dried over MgSO₄, filtered and concentrated under reduced pressure to give substituted 2-bromo-1-(2-bromophenyl)ethanone. 6-R-substituted (R)-(+)-2-Methyl-CBS-oxazaborolidine [For R-isomer] or 6-R-substituted (S)-(−)-2-Methyl-CBS-oxazaborolidine [For S-isomer] (0.11 eq) is added to a stirred solution of substituted 2-bromo-1-(2-bromophenyl)ethanone (1.0 eq) in THF. The reaction mixture is cooled to −10° C. where BH₃.THF (1.0 M in THF, 1.20 eq) is added over 4 hours. The reaction mixture is stirred for a further 45 minutes at −10° C. before the addition of methanol (130 mL). The reaction mixture is concentrated under reduced pressure. The resultant residue is subjected to flash column chromatography to provide the substituted chiral 2-bromo-1-(2-bromophenyl)ethanol. To a solution of this alcohol (1.00 eq) in DMF is added sodium azide at room temperature. The reaction mixture is then heated to 80° C. for 24 hours. Water (150 mL) is added and this solution is extracted with diethyl ether. The combined organic phases are washed with brine (50 mL), dried over MgSO₄, filtered and concentrated under reduced pressure. The residue is subjected to flash column chromatography to yield the substituted 2-azido-1-(2-bromophenyl)ethanol. To a solution of this material (1.00 eq) in toluene is added triisopropyl borate (1.50 eq). The reaction flask is equipped with a Dean and Stark condenser attached and the reaction mixture is refluxed to remove approximately ¾ of the volume of solvent. The dark reaction mixture is cooled to room temperature where THF is added and then cooled to −78° C. n-Butyl lithium (2.5 M in hexanes, 1.15 eq) is added dropwise to the reaction mixture at −78° C. and then stirred for 30 minutes at this temperature. The reaction mixture is then allowed to warm to room temperature where it is stirred for 3 hours before being quenched with 6 M HCl (30 mL). The reaction mixture is concentrated under reduced pressure and the resulting residue is subjected to flash column chromatography to give the 6-R-substituted 3-(azidomethyl)benzo[c][1,2]oxaborol-1(3H)-ol.

To a solution of this compound (1.0 eq) in methanol is added triphenylphosphine (1.0 eq) and this is stirred for 3 hours at room temperature. Concentrated HCl is added and the reaction mixture stirred for a further 2 hours before being concentrated to dryness under reduced pressure. Dichloromethane is added and extracted with 2 M HCl. The combined aqueous layers are washed with dichloromethane before being contracted under reduced pressure. The residue is then recrystalised from hot water/acetonitrile (3 mL water/50-80 mL acetonitrile per gram of compound) to give the substituted chiral (R or S) 6-R-substituted 3-(aminomethyl)benzo[c][1,2]oxaborol-1(3H)-ol as the hydrochloride salt.

### General Procedure for 6-Substituted or unsubstituted phenoxy-3-acetic acid benzoxaborole derivatives

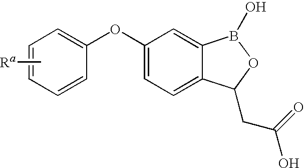

### Step 1

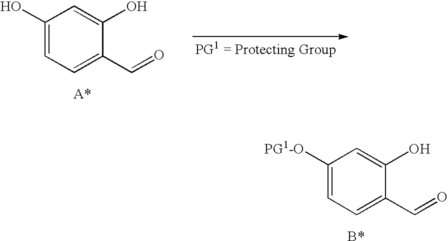

The hydroxyl group of A* can be protected by subjecting the molecule to protecting group appropriate conditions, thereby producing B*.

### Step 2

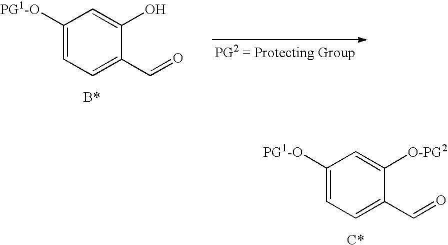

The hydroxyl group of B* can be protected by subjecting the molecule to protecting group appropriate conditions, thereby producing C*.

### Step 3

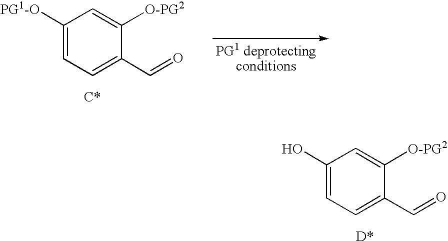

D* can be produced by subjecting C* to conditions that will selectively deprotect PG¹, but not PG².

### Step 4

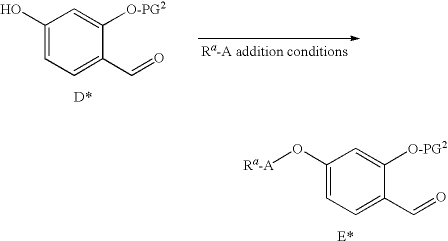

E* can be produced by subjecting D* to conditions that will add R^{a}-A.

### Step 5

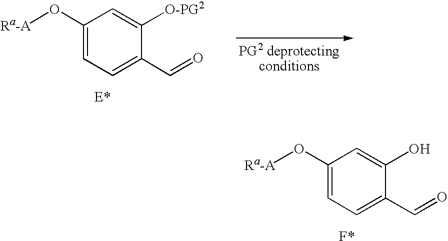

F* can be produced by subjecting E* to conditions that will selectively deprotect PG².

### Step 6

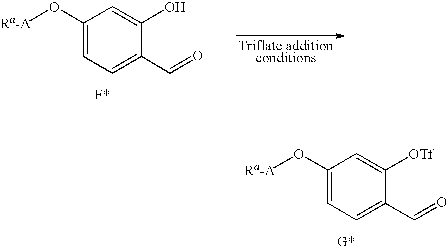

G* can be produced by subjecting F* to conditions that will selectively add a triflate, or a similar group.

### Step 7

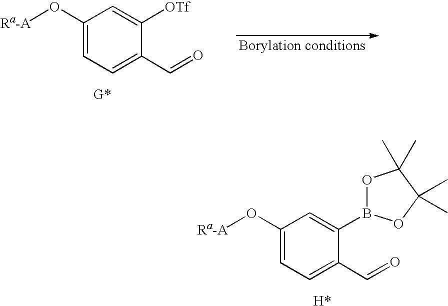

H* can be produced by subjecting G* to borylation conditions.

### Step 8

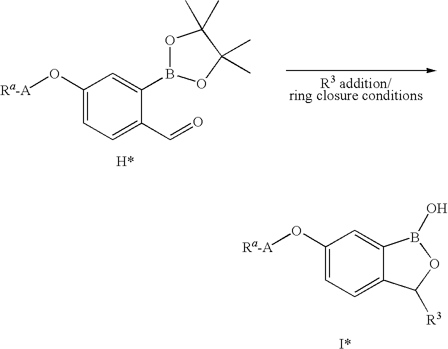

I* can be produced by subjecting H* to R³ addition/ring closure conditions.

### Step 9

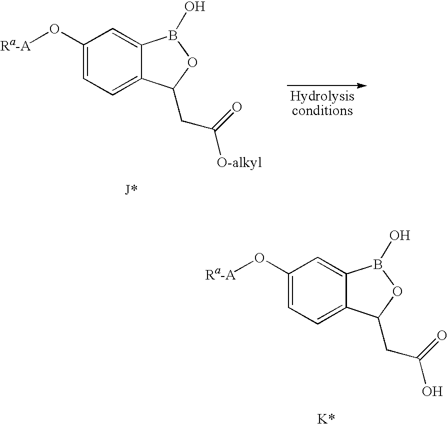

When R³ comprises an ester, for example, J*, the compound can be subjected to hydrolysis conditions to produce K*. The mixture can be purified via precipitation, silica gel column purification or preparative HPLC.

### General Procedure for 6-Substituted or unsubstituted heteroaryl-3-acetic acid benzoxaborole derivatives

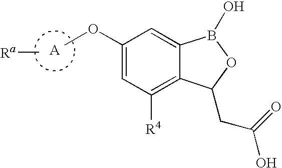

### Step 1.

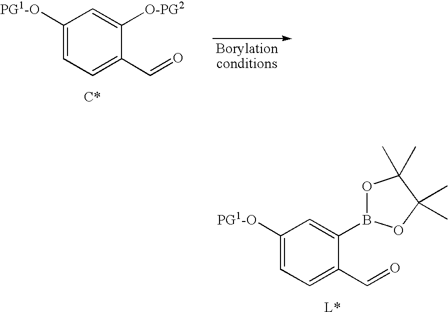

L* can be produced by subjecting C* to borylation conditions.

### Step 3.

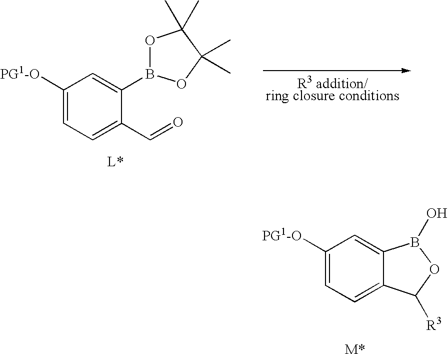

The reaction was carried out using a procedure similar to that described in Step 8 of Strategy A.

### Step 4

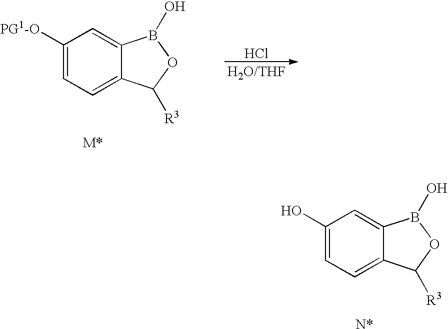

N* can be produced by subjecting M* to conditions that will deprotect PG¹.

### Step 5

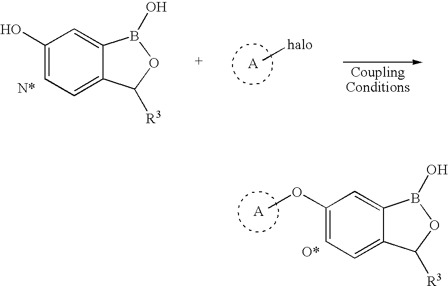

O* can be produced by subjecting N* to appropriate coupling conditions.

### General Procedure for 6-substituted or unsubstituted alkyl-3-acetic acid benzoxaborole derivatives

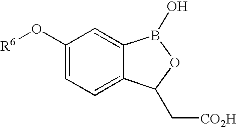

### Step 1

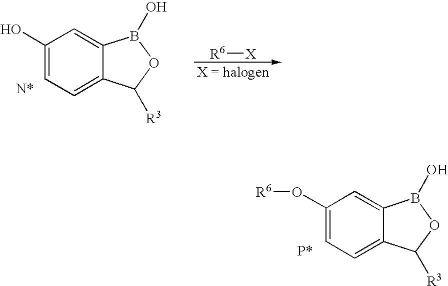

P* can be produced by subjecting N* to appropriate coupling conditions.

### Step 2.

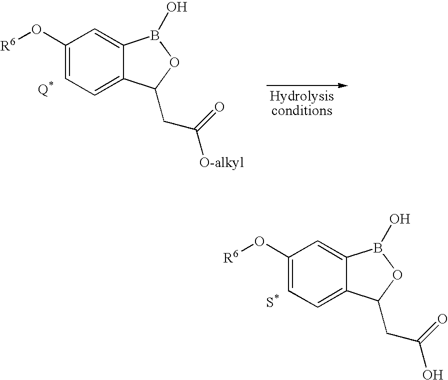

When R³ comprises an ester, for example, Q*, the compound can be subjected to hydrolysis conditions to produce S*. The mixture can be purified via precipitation, silica gel column purification or preparative HPLC.

### General Procedure for 6-substituted-3-propionic acid benzoxaborole derivatives

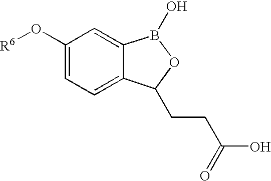

### Step 1:

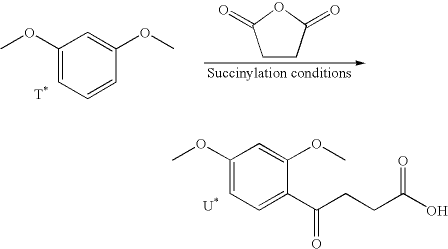

U* can be produced by subjecting T* to succinylation conditions.

### Step 2:

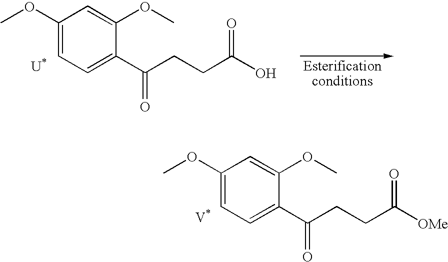

V* can be produced by subjecting U* to esterification conditions.

### Step 3:

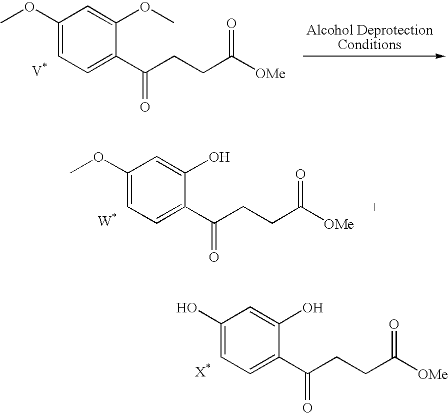

W* or X* can be produced by subjecting V* to alcohol deprotection conditions.

### Step 4:

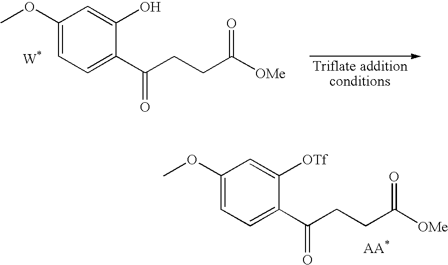

AA** can be produced by subjecting W* to conditions that will selectively add a triflate, or a similar group.

### Step 5:

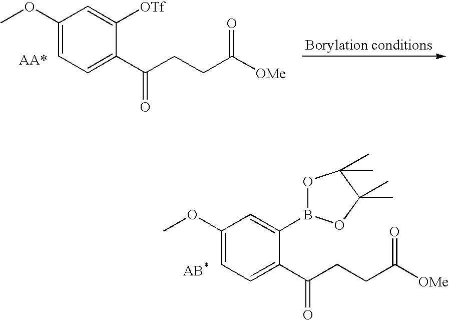

AB* can be produced by subjecting AA* to borylation conditions.

### Step 6:

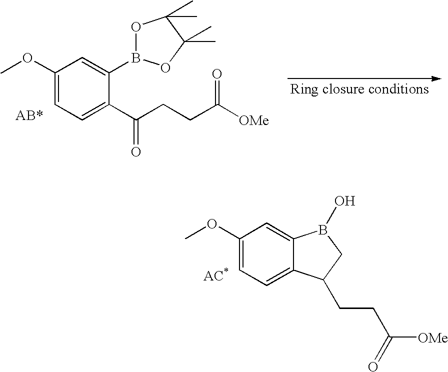

AC* can be produced by subjecting AB* to ring closure conditions.

### General Procedure for Chiral Separation

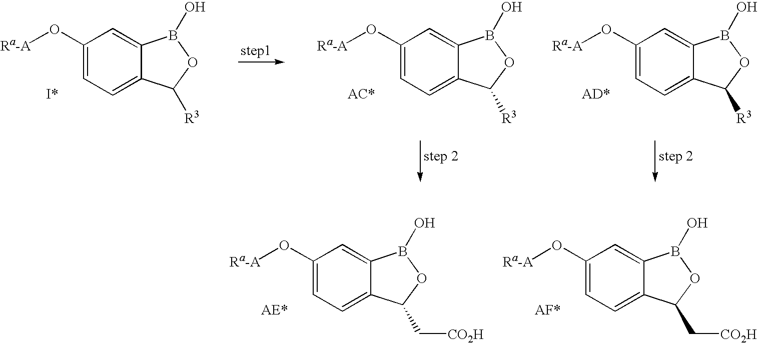

### Step 1

Racemates of compounds such as I* were separated into pure enantiomers via preparative chiral HPLC or preparative supercritical fluid chromatography. Chiral columns which can be utilized to separate compounds of the invention are commercially available from companies such as Chiral Tech (West Chester, Pa.). Exemplary chiral columns which can be utilized include CHIRALPAK® IC, and CHIRALPAK® 405. Solvent systems of use in this purification include CO₂/MeOH (approx 85/15), Hexane/IprOH/TFA Hexane/EtOH/TFA as solvent. EtOH can be replaced with other alcohols.

### Step 2

When R³ comprises an ester, for example, AC* or AD*, the compound can be subjected to hydrolysis conditions to produce compounds such as AE* or AF*. The mixture can be purified via precipitation, silica gel column purification or preparative HPLC.

Compounds of the invention can be produced according to the strategies described herein.

### General Procedure for production of 6-pyridinyloxy compounds

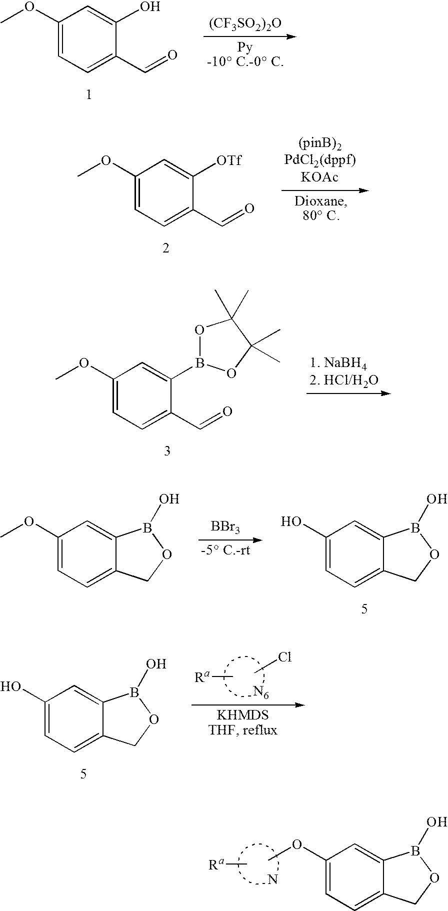

Alternate heterocyclic rings could be introduced through a different selection of 6.

The following general procedure describes a method of introducing a methyl ester moiety at the 3 position of the benzoxaborole.

### General Procedure

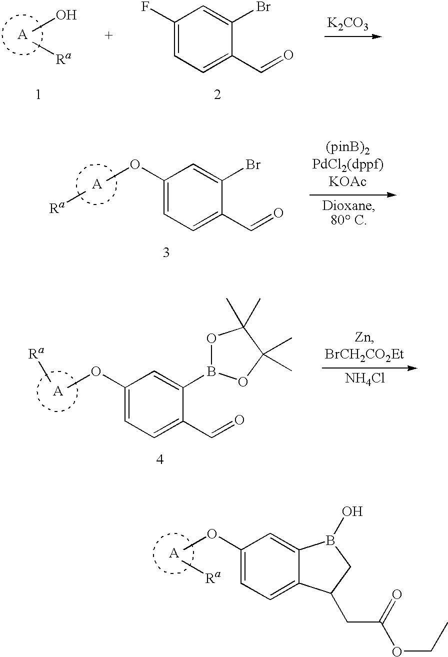

Different substitutions could be accomplished through the addition of a substituent to 1. Variations in the ester could be accomplished through variations of 5.

The following general procedure describes a method of hydrolyzing a methyl ester to a methyl carboxylic acid.

### General Procedure

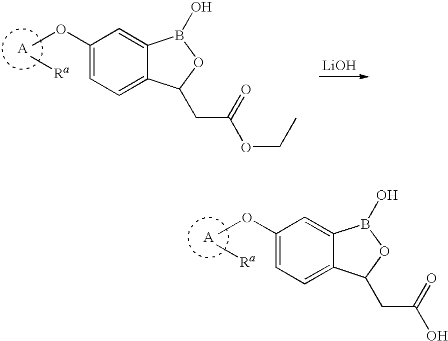

The following general procedure describes a method of hydrolyzing a methyl carboxylic acid to an alkyl alcohol.

### General Procedure

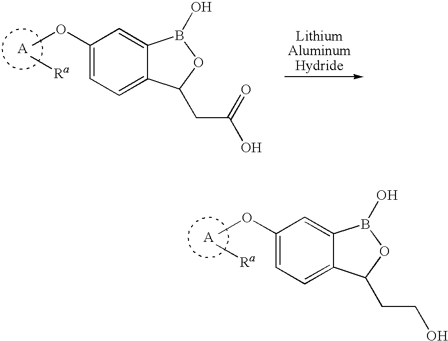

The following general procedure describes a method of making a 6-benzyl substituted benzoxaborole:

### General Procedure

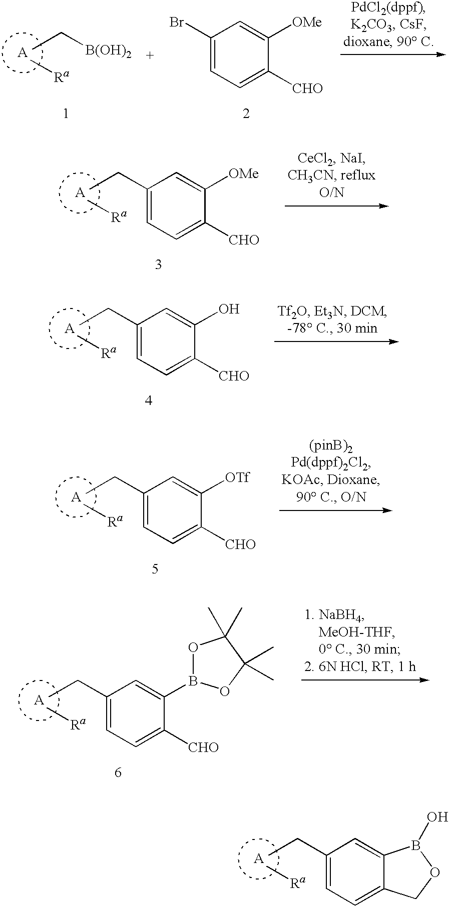

The following general procedure describes a method of making a 3,6 benzoxaborole:

### General Procedure

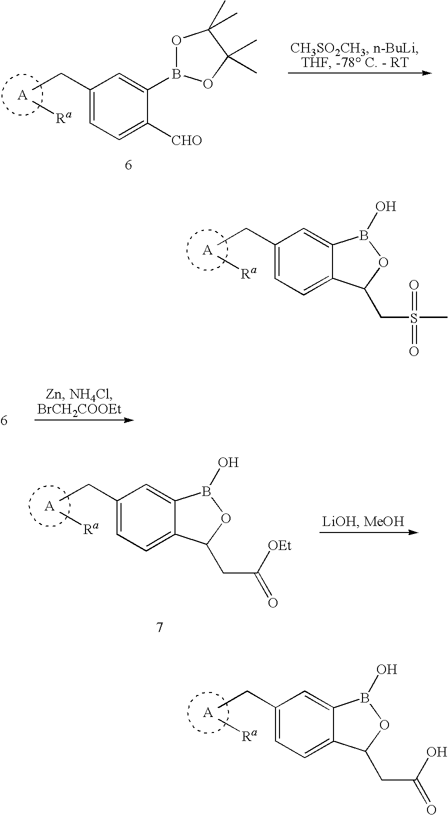

The following general procedure describes a method of making a 3,6 benzoxaborole:

### General Procedure

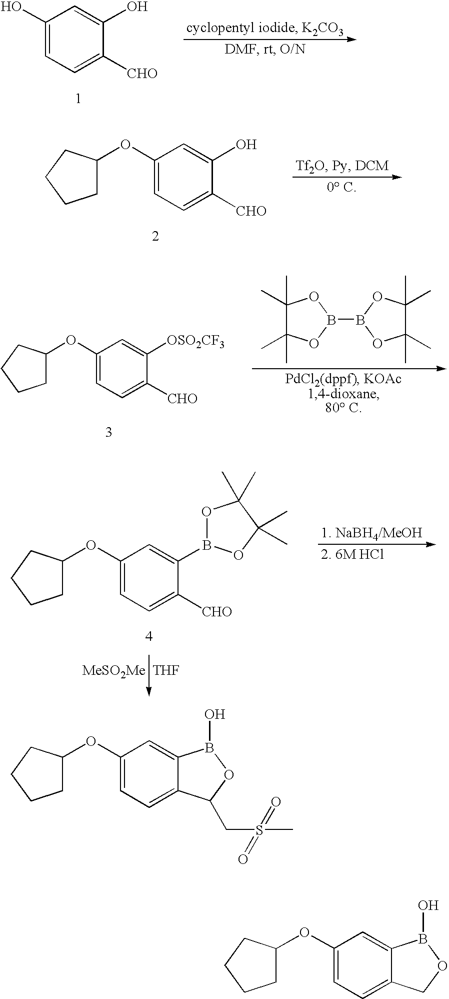

Compounds described herein can be converted into hydrates and solvates by methods similar to those described herein.

### IV. Assays

Art-recognized techniques of genetics and molecular biology are of use to identify compounds that bind to and/or inhibit an enzyme, such as a beta-lactamase or a tRNA synthetase. Moreover, these techniques are of use to distinguish whether a compound binds to and/or inhibits a particular domain of the enzyme. For example, for LeuRS, these techniques can distinguish whether a compound binds to and/or inhibits the synthetic domain, the editing domain, or both the editing and synthetic domains.

### IV. a) Beta-lactamase

In an exemplary assay, activity of a representative compound against a beta-lactamase was confirmed.

Assays to determine whether, and how effectively, a particular compound binds to and/or inhibits a beta-lactamase are also set forth herein, and additional assays are readily available to those of skill in the art.

Generally, the compounds to be tested are present in the assays in ranges from about 1 pM to about 100 mM, preferably from about 1 pM to about 1 μM. Other compounds range from about 1 nM to about 100 nM, preferably from about 1 nM to about 1 μM.

The effects of the test compounds upon the function of the enzymes can also be measured by any suitable physiological change. When the functional consequences are determined using intact cells or animals, one can also measure a variety of effects such as transmitter release, hormone release, transcriptional changes to both known and uncharacterized genetic markers, changes in cell metabolism such as cell growth or pH changes, and changes in intracellular second messengers such as Ca²⁺, or cyclic nucleotides.

High throughput screening (HTS) is also of use in identifying promising candidates of the invention.

Utilizing the assays set forth herein and others readily available in the art, those of skill in the art will be able to readily and routinely determine other compounds and classes of compounds that operate to bind to and/or inhibit a beta-lactamase.

In another aspect, the invention provides a method for identifying a compound which binds a beta-lactamase comprising:
a) contacting said beta-lactamase with a test compound under conditions suitable for binding; and b) detecting binding of said test compound to said beta-lactamase. In an exemplary embodiment, detecting binding of said compound comprises use of at least one detectable element, isotope, or chemical label attached to said compound. In an exemplary embodiment, the element, isotope or chemical label is detected by a fluorescent, luminescent, radioactive, or absorbance readout. In another exemplary embodiment, wherein said beta-lactamase comprises the amino acid sequence of a peptide sequence described herein.

In another aspect, the invention provides a method for identifying a compound which binds to a beta-lactamase, said assay comprising: a) contacting said beta-lactamase with said compound under conditions suitable for binding of said compound with said beta-lactamase; b) comparing a biological activity of said beta-lactamase contacting said compound to said biological activity when not contacting said compound; and c) identifying said compound as binding to said beta-lactamase if said biological activity of said beta-lactamase is reduced when contacting said compound.

### IV. b) LeuRS

In an exemplary assay, activity of a representative compound against the editing domain was confirmed. To identify the target of a novel boron-containing antibacterial compound, mutants in E. coli showing resistance to the compound were isolated. Characterization of mutants showed that they have an 32-256 fold increase in resistance to the compound over wildtype. The mutants were furthermore shown to be sensitive to various antibacterial agents with known modes of action, suggesting that the cellular target of the compound is distinct from the target of the other antibacterial agents. The leuS gene from the mutants was cloned onto a plasmid and their resistance was confirmed by MIC. The editing domain from these mutants were sequenced and the mutations were all located in the editing domain of this enzyme.

Assays to determine whether, and how effectively, a particular compound binds to and/or inhibits the editing domain of a selected tRNA synthetase are also set forth herein, and additional assays are readily available to those of skill in the art. Briefly, in an exemplary assay, an improperly charged tRNA and a tRNA synthetase that is capable of editing the improperly charged tRNA are combined. The resulting mixture is contacted with the putative inhibitor and the degree of editing inhibition is observed.

Another assay uses genetics to show that a drug works via the editing domain. In this assay, the compound is first tested against a strain of cells over-expressing copies of the tRNA synthetase gene. The compound's effect on the over-expressing strain is compared with a control strain to determine whether the compound is active against the synthetase. If the minimum inhibitory concentration (MIC) is 2-fold higher in the strain with extra copies of the synthetase gene than the MIC of the inhibitor against a wild type cell, a further genetic screen is conducted to determine whether the increased resistance is due to mutations in the editing domain. In this second screen, the control strain is challenged against a high concentration of the inhibitor. The colonies surviving the challenge are isolated and DNA from these cells is isolated. The editing domain is amplified using a proof-reading PCR enzyme and the appropriate primers. The PCR product can be purified using standard procedures. The sequence amplified mutant DNA is compared to wild-type. If the mutant DNA bears mutations in the editing domain, such results would suggest that the compound binds to the editing domain and affects the editing function of the molecule through this domain.

Generally, the compounds to be tested are present in the assays in ranges from about 1 pM to about 100 mM, preferably from about 1 pM to about 1 μM. Other compounds range from about 1 nM to about 100 nM, preferably from about 1 nM to about 1 μM.

The effects of the test compounds upon the function of the enzymes can also be measured by any suitable physiological change. When the functional consequences are determined using intact cells or animals, one can also measure a variety of effects such as transmitter release, hormone release, transcriptional changes to both known and uncharacterized genetic markers, changes in cell metabolism such as cell growth or pH changes, and changes in intracellular second messengers such as Ca²⁺, or cyclic nucleotides.

High throughput screening (HTS) is also of use in identifying promising candidates of the invention.

Utilizing the assays set forth herein and others readily available in the art, those of skill in the art will be able to readily and routinely determine other compounds and classes of compounds that operate to bind to and/or inhibit the editing domain of tRNA synthetases.

In another aspect, the invention provides a method for identifying a compound which binds to an editing domain of a tRNA synthetase comprising: a) contacting said editing domain with a test compound under conditions suitable for binding; and b) detecting binding of said test compound to said editing domain. In an exemplary embodiment, detecting binding of said compound comprises use of at least one detectable element, isotope, or chemical label attached to said compound. In an exemplary embodiment, the element, isotope or chemical label is detected by a fluorescent, luminescent, radioactive, or absorbance readout. In an exemplary embodiment, the contacting of said test compound with said editing domain also includes further contacting said test compound and said editing domain with a member selected from AMP and a molecule with a terminal adenosine. In an exemplary embodiment, the tRNA synthetase is derived from leucyl tRNA synthetase. In an exemplary embodiment, the tRNA synthetase is derived from a mutated tRNA synthetase, wherein said mutated tRNA synthetase comprises amino acid mutations in an editing domain. In another exemplary embodiment, wherein said editing domain of a tRNA synthetase comprises the amino acid sequence of a peptide sequence described herein.

In another aspect, the invention provides a method for identifying a compound which binds to an editing domain of a tRNA synthetase, said assay comprising: a) contacting said editing domain of a tRNA synthetase with said compound under conditions suitable for binding of said compound with said editing domain of a tRNA synthetase; b) comparing a biological activity of said editing domain of a tRNA synthetase contacting said compound to said biological activity when not contacting said compound; and c) identifying said compound as binding to said editing domain of a tRNA synthetase if said biological activity of said editing domain of a tRNA synthetase is reduced when contacting said compound. In an exemplary embodiment, the biological activity is hydrolysis of noncognate amino acid. In another exemplary embodiment, the hydrolysis of said noncognate amino acid is detected through the use of one or more labels. In another exemplary embodiment, the labels include a radiolabel, a fluorescent marker, an antibody, or a combination thereof. In another exemplary embodiment, said labels can be detected using spectroscopy. In another exemplary embodiment, said editing domain of a tRNA synthetase is derived from leucyl tRNA synthetase.

In another aspect, the invention provides a method of generating tRNA molecules with noncognate amino acid comprising: a) creating or isolating a mutated tRNA synthetase with altered amino acid editing domains; and b) contacting a tRNA molecule with said mutated tRNA synthetase and a noncognate amino acid. In another exemplary embodiment, the mutated tRNA synthetase contains one or more amino acid mutations in an editing domain. In another exemplary embodiment, the mutated tRNA synthetase is unable to bind with a compound of the invention. In another exemplary embodiment, the mutated tRNA synthetase is unable to bind with a compound described herein, or a pharmaceutically acceptable salt thereof. In another exemplary embodiment, the mutated tRNA synthetase is unable to bind with a compound according to a formula described herein, or a pharmaceutically acceptable salt thereof.

In another aspect, the invention provides a composition that comprises one or more tRNA molecules attached to noncognate amino acids, wherein said tRNA molecules are synthesized using one or more mutated tRNA synthetases isolated from a microorganism or a cell line derived from a microorganism. In an exemplary embodiment, the microorganism is a bacteria. In an exemplary embodiment, wherein said mutated tRNA synthetases contain amino acid mutations in their editing domains.

### V. Amino Acid and Nucleotide Sequences Used in Assays

tRNA Sequences that Interact with the tRNA Synthetase-Compound of the Invention-AMP Complex

Transfer RNAs (tRNAs) translate mRNA into a protein on a ribosome. Each transfer RNA contains an anti-codon region that hybridizes with mRNA, and an amino acid which may be attached to the growing peptide. The structural gene of tRNA is about 72 to 90 nucleotides long and folds into a cloverleaf structure (Sharp S. J., Schaack J., Coolen L., Burke D. J. and Soll D., “Structure and transcription of eukaryotic tRNA genes”, Crit. Rev. Biochem, 19:107 144 (1985); Geiduschek E. O., and Tocchini-Valentini, “Transcription by RNA polymerase III”, Annu. Rev. Biochem. 57:873 914 (1988)).

In one embodiment, a compound described herein contacts AMP and a tRNA synthetase, and the tRNA synthetase in turn contacts a tRNA molecule. In another embodiment, a compound described herein contacts AMP from the tRNA molecules and a tRNA synthetase. The nucleotide sequence of the tRNA molecule can be determined by the identity of the tRNA synthetase involved. For example, for leucyl tRNA synthetase, the cognate tRNA molecule bound will be tRNA-leucine (SEQ ID NO: 1), but a noncognate tRNA, such as isoleucine, (SEQ ID NO: 2) may be bound under certain conditions. In another embodiment, the tRNA molecule is a leucyl t-RNA. In another embodiment, the tRNA molecule is represented by a SEQ ID described herein. In another embodiment, the tRNA molecule is represented by SEQ ID NO: 14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23 and SEQ ID NO:24. In this and other embodiments, the term “noncognate” is meant to encompass both the singular and plural forms of the word, i.e. the phrase “noncognate amino acid” comprises one or more amino acids. In the following sequences; s4U=s⁴U; 4-thiouridine; Gm=methylguanine; Y=pyrimidine; ms2i6A=ms²i⁶A; 2-methylthio-N-6-isopentenyl adenosine and D=dihydrouridine.

SEQ ID NO: 1 corresponds to the nucleotide sequence of the tRNA-Leu gene from Saccharomyces cerevisiae:

gggagtttgg ccgagtggtt taaggcgtca gatttaggct
ctgatatctt cggatgcaagggttcgaatc ccttagctct cacca

SEQ ID NO: 2 corresponds to the nucleotide sequence of the tRNA-Ile gene from Saccharomyces cerevisiae:

gaaactataa ttcaattggt tagaatagta ttttgataag
gtacaaatat aggttcaatc cctgttagtt tcatcca

SEQ ID NO: 14 corresponds to the nucleotide sequence of a tRNA-Leu gene from E. coli:

gcgaaggtggcggaattggtagacgcgctagcttcaggtgttagtgtcct
tacggacgtgggggttcaagtcccccccctcgcacca

SEQ ID NO: 15 corresponds to the nucleotide sequence of a tRNA-Leu gene from E. coli:

gcgggagtggcgaaattggtagacgcaccagatttaggttctggcgccgc
aaggtgtgcgagttcaagtctcgcctcccgcacca

SEQ ID NO: 16 corresponds to the nucleotide sequence of a tRNA-Leu gene from E. coli:

gccgaagtggcgaaatcggtagacgcagttgattcaaaatcaaccgtaga
aatacgtgccggttcgagtccggccttcggcacca

SEQ ID NO: 17 corresponds to the nucleotide sequence of a tRNA-Leu gene from E. coli:

gccgaggtggtggaattggtagacacgctaccttgaggtggtagtgccca
atagggcttacgggttcaagtcccgtcctcggtacca

SEQ ID NO: 18 corresponds to the nucleotide sequence of a tRNA-Leu gene from E. coli:

gcccggatggtggaatcggtagacacaagggatttaaaatccctcggcgt
tcgcgctgtgcgggttcaagtcccgctccgggtacca

SEQ ID NO: 19 corresponds to the nucleotide sequence of a tRNA-Leu gene from E. coli:

GCCCGGAs4UGGUGGAADCGmGDAGACACAAGGGAYUunkAAAms2i6AA
YCCCUCGGCGUUCGCGCUGUGCGGGTYCAAGUCCCGCUCCGGGUACCA

SEQ ID NO: 20 corresponds to the nucleotide sequence of a tRNA-Leu gene from E. coli:

GCGAAGGUGGCGGAADDGmGDAGACGCGCUAGCUUCAGunkGYGYUAGUG
UCCUUACGGACGUGGGGGTYCAAGUCCCCCCCCUCGCACCA

SEQ ID NO: 21 corresponds to the nucleotide sequence of a tRNA-Leu gene from E. coli:

GCCGAGGUGGUGGAADDGmGDAGACACGCUACCUUGAGunkGYGGUAGUG
CCCAAUAGGGCUUACGGGTYCAAGUCCCGUCCUCGGUACCA

SEQ ID NO: 22 corresponds to the nucleotide sequence of a tRNA-Leu gene from Pseudomonas aeruginosa

gcggacgtggtggaattggtagacacactggatttaggttccagcgccgc
aaggcgtgagagttcgagtctctccgtccgcacca

SEQ ID NO: 23 corresponds to the nucleotide sequence of a tRNA-Leu gene from Staphylococcus aureus

gccggggtggcggaactggcagacgcacaggacttaaaatcctgcggtga
gagatcaccgtaccggttcgattccggtcctcggcacca

SEQ ID NO: 24 corresponds to the nucleotide sequence of a tRNA-Leu gene from Staphylococcus aureus

gccggggtggcggaactggcagacgcacaggacttaaaatcctgcggtga
gtgatcaccgtaccggttcgattccggtcctcggcacca

Polypeptides Used in Binding and Inhibition Assays

In some binding and inhibition assays, it is more effective to use a portion of a tRNA synthetase molecule rather than the whole protein itself. In such assays, polypeptides derived from tRNA synthetases are used in the experiment.

In one preferred embodiment, polypeptide fragments corresponding to the editing domain of a tRNA synthetase molecule are used in assay and binding experiments. Such fragments are represented by SEQ ID NO:3, SEQ ID NO:4, SEQ ID NO:5, SEQ ID NO:6 and SEQ ID NO:7. In an exemplary embodiment, the fragments are represented by SEQ ID NO:5, SEQ ID NO:6 and SEQ ID NO:7.

SEQ ID NO 3:
TPQEYIGVKIEALEFADDAAKIIDSSSDLDKSKKFYFVAATLRPETMYGQ
TCCFVSPTIEYGIFDAGDSYFITTERAFKNMSYQKLTPKRGFYKPIVTVP
GKAFIGTKIHAPQSVYPELRILPMETVIATKGTGVVTCVPSNSPDDYITT
KDLLHKPEYYGIKPEWIDHEIVPIMHTEKYGDLTAKAIVEEKKIQSPKDK
NLLAEAKKIAYKEDYYTGTMIYGPYKGEKVEQAKNKVKADMIAAGEAFVY
NEPESQDP
SEQ ID NO 4:
MTPQEYIGVKIEALEFADDAAKIIDSSSDLDKSKKFYFVAATLRPETMYG
QTCCFVSPTIEYGIFDAGDSYFITTERAFKNMSYQKLTPKRGFYKPIVTV
PGKAFIGTKIHAPQSVYPELRILPMETVIATKGTGVVTCVPSNSPDDYIT
TKDLLHKPEYYGIKPEWIDHEIVPIMHTEKYGDLTAKAIVEEKKIQSPKD
KNLLAEAKKIAYKEDYYTGTMIYGPYKGEKVEQAKNKVKADMIAAGEAFV
YNEPESQDPQDPNSSSVDKLAAALEHHHHH
SEQ ID NO 5:
TCTPEYYRWEQKFFTELYKKGLVYKKTSAVNWCPNDQTVLANEQVIDGCC
WRCDTKVERKEIPQWFIKITAYADELLNDLDKLDHWPDTVKTMQRNWIGR
SEGVEITFNVNDYDNTLTVYTTRPDTFMGCTYLAVAAGHPLAQKAAENNP
ELAAFIDECRNTKVAEAEMATMEKKGVDTGFKAVHPLTGEEIPVWAANFV
LMEYGTGAVMAVPGHDQRDYEFASKYGLNIKPVILAADGSEPDLSQQALT
EKGVLFNSGEFNGLDHEAAFNAIADKLTAMGVGERKVNYRLRDWGVSRQR
YWG
SEQ ID NO 6:
TCKPDYYRWEQWLFTRLFEKGVIYRKNGTVNWDPADQTVLANEQVIDGRG
WRSGALIEKREIPMYYFRITDYADELLESLDELPGWPEQVKTMQRNWIGK
SRGMEVQFPYDQASIGHEGTLKVFTTRPDTLMGATYVAVAAEHPLATQAA
QGNAALQAFIDECKSGSVAEADMATQEKKGMATSLFVEHPLTGEKLPVWV
ANYVLMHYGDGAVMAVPAHDERDFEFAHKYNLPVKAVVRTSAGDDVGSEW
LAAYGEHGQLINSGEFDGLDFQGAFDAIEAALIRKDLGKSRTQFRLRDWG
ISRQRYWG
SEQ ID NO 7:
TTDPEYYKWTQWIFIQLYNKGLAYVDEVAVNWCPALGTVLSNEEVIDGVS
ERGGHPVYRKPMKQWVLKITEYADQLLADLDDLDWPESLKDMQRNWIGRS
EGAKVSFDVDNTEGKVEVFTTRPDTIYGASFLVLSPEHALVNSITTDEYK
EKVKAYQTEASKKSDLERTDLAKDKSGVFTGAYAINPLSGEKVQIWIADY
VLSTYGTGAIMAVPAHDDRDYEFAKKFDLLIIEVIEGGNVEEAAYTGEGK
HINSGELDGLENEAAITKAIQLLEQKGAGEKKVYKLRDWLFSRQRYWG

SEQ ID NO 8 corresponds to a peptide sequence for a leu-tRNA synthetase editing domain for Escherichia coli

GRSEGVEITFNVNDYDNTLTVYTTRPDTFMGCTYLAVAAGHPLAQKAAEN
NPELAAFIDECRNTKVAEAEMATMEKKGVDTGFKAVHPLTGEEIPVWAAN
FVLMEYGTGAVMAVPGHDQRDYEFASKYGLNIKPVILAADGSEPDLSQQA
LTEKGVLFNSGEFNGLDHEAAFNAIADKLTAMGVGERKVNYR

SEQ ID NO 9 corresponds to a peptide sequence for a leu-tRNA synthetase editing domain for Pseudomonas

GKSRGMEVQFPYDQASIGHEGTLKVFTTRPDTLMGATYVAVAAEHPLATQ
AAQGNAALQAFIDECKSGSVAEADMATQEKKGMATSLFVEHPLTGEKLPV
WVANYVLMHYGDGAVMAVPAHDERDFEFAHKYNLPVKAVVRTSAGDDVGS
EWLAAYGEHGQLINSGEFDGLDFQGAFDAIEAALIRKDLGKSRTQFR

SEQ ID NO 10 corresponds to a peptide sequence for a leu-tRNA synthetase editing domain for Staphylococcus aureus

GRSEGAKVSFDVDNTEGKVEVFTTRPDTIYGASFLVLSPEHALVNSITTD
EYKEKVKAYQTEASKKSDLERTDLAKDKSGVFTGAYAINPLSGEKVQIWI
ADYVLSTYGTGAIMAVPAHDDRDYEFAKKFDLLIIEVIEGGNVEEAAYTG
EGKHINSGELDGLENEAAITKAIQLLEQKGAGEKKVYK

In one preferred embodiment, polypeptides corresponding to a tRNA synthetase molecule are used in assay and binding experiments. Such polypeptides are represented by SEQ ID NO:11, SEQ ID NO:12 and SEQ ID NO:13.

SEQ ID NO 11 corresponds to a peptide sequence for a leu-tRNA synthetase for Escherichia coli

MQEQYRPEEIESKVQLHWDEKRTFEVTEDESKEKYYCLSMLPYPSGRLHM
GHVRNYTIGDVIARYQRMLGKNVLQPIGWDAFGLPAEGAAVKNNTAPAPW
TYDNIAYMKNQLKMLGFGYDWSRELATCTPEYYRWEQKFFTELYKKGLVY
KKTSAVNWCPNDQTVLANEQVIDGCCWRCDTKVERKEIPQWFIKITAYAD
ELLNDLDKLDHWPDTVKTMQRNWIGRSEGVEITFNVNDYDNTLTVYTTRP
DTFMGCTYLAVAAGHPLAQKAAENNPELAAFIDECRNTKVAEAEMATMEK
KGVDTGFKAVHPLTGEEIPVWAANFVLMEYGTGAVMAVPGHDQRDYEFAS
KYGLNIKPVILAADGSEPDLSQQALTEKGVLFNSGEFNGLDHEAAFNAIA
DKLTAMGVGERKVNYRLRDWGVSRQRYWGAPIPMVTLEDGTVMPTPDDQL
PVILPEDVVMDGITSPIKADPEWAKTTVNGMPALRETDTFDTFMESSWYY
ARYTCPQYKEGMLDSEAANYWLPVDIYIGGIEHAIMHLLYFRFFHKLMRD
AGMVNSDEPAKQLLCQGMVLADAFYYVGENGERNWVSPVDAIVERDEKGR
IVKAKDAAGHELVYTGMSKMSKSKNNGIDPQVMVERYGADTVRLFMMFAS
PADMTLEWQESGVEGANRFLKRVWKLVYEHTAKGDVAALNVDALTENQKA
LRRDVHKTIAKVTDDIGRRQTFNTAIAAIMELMNKLAKAPTDGEQDRALM
QEALLAVVRMLNPFTPHICFTLWQELKGEGDIDNAPWPVADEKAMVEDST
LVVVQVNGKVRAKITVPVDATEEQVRERAGQEHLVAKYLDGVTVRKVIYV
PGKLLNLVVG

SEQ ID NO 12 corresponds to a peptide sequence for a leu-tRNA synthetase for Pseudomonas

MHEQYTPRDVEAAAQNAWDEQQSFAVTEQPGKETYYCLSMFPYPSGKLHM
GHVRNYTIGDVIARYQRMLGKNVLQPMGWDAFGMPAENAAMKNNVAPAKW
TYENIDYMKTQLKSLGLAIDWSREVTTCKPDYYRWEQWLFTRLFEKGVIY
RKNGTVNWDPADQTVLANEQVIDGRGWRSGALIEKREIPMYYFRITDYAD
ELLESLDELPGWPEQVKTMQRNWIGKSRGMEVQFPYDQASIGHEGTLKVF
TTRPDTLMGATYVAVAAEHPLATQAAQGNAALQAFIDECKSGSVAEADMA
TQEKKGMATSLFVEHPLTGEKLPVWVANYVLMHYGDGAVMAVPAHDERDF
EFAHKYNLPVKAVVRTSAGDDVGSEWLAAYGEHGQLINSGEFDGLDFQGA
FDAIEAALIRKDLGKSRTQFRLRDWGISRQRYWGCPIPIIHCPSCGDVPV
PEDQLPVTLPENVVPDGAGSPLARMPEFYECTCPKCGTAAKRETDTMDTF
VESSWYFARYASPNYDKGLVDPKAANHWLPVDQYIGGIEHAILHLLYARF
FHKLMRDEGLVTSNEPFKNLLTQGMVVAETYYRVASNGGKDWFNPADVEI
ERDAKAKIIGARLKTDGLPVEIGGTEKMSKSKNNGVDPQSMIEQYGADTC
RLFMMFASPPDMSLEWSDSGVEGASRFLRRVWRLAQAHVAQGLPGQLDIA
ALSDEQKVIRRAIHAAIKQASTDVGQFHKFNTAIAQVMTVMNVLEKAPQV
TAQDRALLQEGLEAVTLLLAPITPHISHELWKQLGHEQAVIDATWPSVDE
SALVQDTVTLVVQVNGKLRGQVEMPAAASREEIEAAARNNENVLRFTDGL
TIRKVIVVPGKLVNIVAN

SEQ ID NO 13 corresponds to a peptide sequence for a leu-tRNA synthetase for Staphylococcus aureus

MNYNHNQIEKKWQDYWDENKTFKTNDNLGQKKFYALDMFPYPSGAGLHVG
HPEGYTATDIISRYKRMQGYNVLHPMGWDAFGLPAEQYALDTGNDPREFT
KKNIQTFKRQIKELGFSYDWDREVNTTDPEYYKWTQWIFIQLYNKGLAYV
DEVAVNWCPALGTVLSNEEVIDGVSERGGHPVYRKPMKQWVLKITEYADQ
LLADLDDLDWPESLKDMQRNWIGRSEGAKVSFDVDNTEGKVEVFTTRPDT
IYGASFLVLSPEHALVNSITTDEYKEKVKAYQTEASKKSDLERTDLAKDK
SGVFTGAYAINPLSGEKVQIWIADYVLSTYGTGAIMAVPAHDDRDYEFAK
KFDLLIIEVIEGGNVEEAAYTGEGKHINSGELDGLENEAAITKAIQLLEQ
KGAGEKKVNYKLRDWLFSRQRYWGEPIPVIHWEDGTMTTVPEEELPLLLP
ETDEIKPSGTGESPLANIDSFVNVVDEKTGMKGRRETNTMPQWAGSCWYY
LRYIDPKNENMLADPEKLKHWLPVDLYIGGVEHAVLHLLYARFWHKVLYD
LGIVPTKEPFQKLFNQGMILGEGNEKMSKSKGNVINPDDIVQSHGADTLR
LYEMFMGPLDAAIAWSEKGLDGSRRFLDRVWRLIVNEDGTLSSKIVTTNN
KSLDKVYNQTVKKVTDDFETLGFNTAISQLMVFINECYKVDEVYKPYIEG
FVKMLAPIAPHIGEELWSKLGHEESITYQPWPTYDEALLVDDEVEIVVQV
NGKLRAKIKIAKDTSKEEMQEIALSNDNVKASIEGKDIMKVIAVPQKLVN
IVAK

### VI. Methods

In another aspect, the compounds of the invention can be utilized to inhibit an enzyme. In another aspect, the compounds of the invention and/or combinations of the invention exhibit potency against microorganisms, such as bacteria, and therefore have the potential to kill and/or inhibit the growth of microorganisms. In another aspect, the compounds of the invention and/or combinations of the invention exhibit potency against microorganisms, such as bacteria, and therefore have the potential to achieve therapeutic efficacy in the animals described herein.

### VI. a) Beta-Lactamase

In an exemplary embodiment, the compounds of the invention exhibit the ability to inhibit a beta-lactamase, and therefore have the potential to be used to treat bacterial infections in man which involve beta-lactamases. According to another aspect of the invention, a method for binding to and/or inhibiting a beta-lactamase is provided which comprises contacting the beta-lactamase with an effective amount of a compound of the invention. Such conditions are known to those skilled in the art. In an exemplary embodiment, the compound of use in the method is described herein, or a salt, hydrate or solvate thereof, or a combination thereof. In an exemplary embodiment, the compound of use in the method is described herein, or a salt, hydrate or solvate thereof. In an exemplary embodiment, the compound of use in the method is described herein, or a salt thereof. In an exemplary embodiment, the compound of use in the method is described herein, or a salt thereof. The beta-lactamase is contacted with an amount of a compound of the invention sufficient to result in a detectable amount of beta-lactamase inhibition. This method can be performed on a beta-lactamase that is contained within an organism or which is outside an organism. In an exemplary embodiment, the method is performed on a beta-lactamase that is contained within a microorganism that is in, or on the surface of, an animal. In an exemplary embodiment, the animal is a human. In an exemplary embodiment, the inhibition takes place in a cell, such as a microorganism cell. In another exemplary embodiment, the microorganism is a bacteria. In an exemplary embodiment, the method is performed on a beta-lactamase that is outside of a microorganism. In an exemplary embodiment, the method is performed on a beta-lactamase that is outside of a microorganism and is in an assay of the type described herein.

In an exemplary embodiment, the compound has a structure according to the following formula:

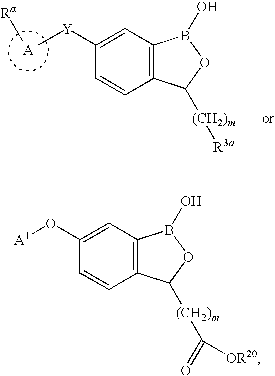

in which Y, A, R^{a}, m and R^{3a} is described herein. In an exemplary embodiment, the compound has a structure according to the following formula:

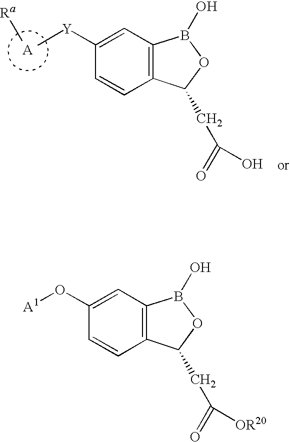

in which Y, A, R²⁰ and R^{a} are described herein. In an exemplary embodiment, the β-lactamase is a member selected from a Group 1 β-lactamase, a Group 2 β-lactamase, a Group 3 β-lactamase, and a Group 4 β-lactamase. In an exemplary embodiment, the Group 1 β-lactamase is a cephalosporinase. In an exemplary embodiment, said Group 2 β-lactamase is a member selected from penicillinase, a Group 2b, Group 2be, Group 2br, carbenicillinase, cloxacilanase, cephalosporinase and carbapenamase. In an exemplary embodiment, said Group 3 β-lactamase is a metallo-β-lactamase. In an exemplary embodiment, said Group 4 β-lactamase is a penicillinase. In an exemplary embodiment, the β-lactamase is a member selected from a class A β-lactamase, a class B β-lactamase, a class C β-lactamase, and a class D β-lactamase. In an exemplary embodiment, the class A β-lactamase is a member selected from a TEM β-lactamase, SHV β-lactamase, CTX-M β-lactamase and a KPC β-lactamase. In an exemplary embodiment, β-lactamase is TEM β-lactamase. In an exemplary embodiment, the β-lactamase is TEM-1 β-lactamase. In an exemplary embodiment, the β-lactamase is TEM-3 β-lactamase. In an exemplary embodiment, the β-lactamase is KPC-2 β-lactamase. In an exemplary embodiment, the β-lactamase is CMY-2 β-lactamase. In an exemplary embodiment, the class C β-lactamase is a member selected from a CMY β-lactamase, a PER β-lactamase and an AmpC β-lactamase. In an exemplary embodiment, the β-lactamase is AmpC β-lactamase. In an exemplary embodiment, the class D β-lactamase is an OXA β-lactamase. In an exemplary embodiment, the β-lactamase is a metallo β-lactamase. In an exemplary embodiment, the metallo β-lactamase is a member selected from an IMP carbapenemase and a VIM β-lactamase. In an exemplary embodiment, the β-lactamase is a member selected from a class A β-lactamase and a class C β-lactamase. In an exemplary embodiment, the contacting takes place in vitro. In an exemplary embodiment, the contacting takes place in vitro. In an exemplary embodiment, the contacting takes place in an animal, such as a human.

### VI. b) LeuRS

In an exemplary embodiment, the compounds of the invention exhibit the ability of inhibiting the editing domain of tRNA synthetases, such as leucyl tRNA synthetase, of microorganisms, such as bacteria, and therefore have the potential to be used as editing domain inhibitors of microorganism tRNA synthetases.

According to another aspect of the invention, a method for binding to and/or inhibiting the editing domain of a tRNA synthetase is provided which comprises contacting a tRNA synthetase with a compound of the invention that inhibits the editing domain under conditions in which the tRNA synthetase interacts with its substrate to form an aminoacyl adenylate intermediate and, preferably, to form a charged tRNA. Such conditions are known to those skilled in the art. In an exemplary embodiment, the compound has a structure according to the following formula:

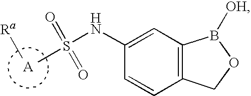

in which A and R^{a} is described herein. In an exemplary embodiment, the compound is E111 or a salt thereof. In an exemplary embodiment, the compound is E111 or a pharmaceutically acceptable salt thereof. In an exemplary embodiment, the compound is E119 or a salt thereof. In an exemplary embodiment, the compound is E119 or a pharmaceutically acceptable salt thereof. In an exemplary embodiment, the compound is described herein, or a salt, hydrate or solvate thereof, or a combination thereof. In an exemplary embodiment, the invention provides a compound described herein, or a salt, hydrate or solvate thereof. In an exemplary embodiment, the invention provides a compound described herein, or a salt thereof. In an exemplary embodiment, the invention provides a compound described herein, or a salt thereof The tRNA synthetase is contacted with an amount of compound of the invention sufficient to result in a detectable amount of tRNA synthetase inhibition. This method can be performed on a tRNA synthetase that is contained within an organism or which is outside an organism. In an exemplary embodiment, the method is performed on a tRNA synthetase that is contained within a microorganism or a microbial cell that is in, or on the surface of, an animal. In an exemplary embodiment, the animal is a human. The method results in a decrease in the amount of charged tRNA produced by the tRNA synthetase that has an inhibited editing domain. In an exemplary embodiment, the inhibition takes place in a cell, such as a microorganism cell. In another exemplary embodiment, the microorganism cell is a bacteria. In another exemplary embodiment, the tRNA synthetase is leucyl tRNA synthetase.

In an exemplary embodiment, the invention provides a method of inhibiting conversion of a tRNA molecule into a charged tRNA molecule. The method involves contacting a tRNA synthetase with a compound of the invention effective to inhibit activity of an editing domain of said tRNA synthetase, under conditions sufficient to inhibit said activity, thereby inhibiting said conversion. In an exemplary embodiment, the compound of the invention is a compound described herein, or a pharmaceutically acceptable salt thereof. In an exemplary embodiment, the inhibition occurs within a cell, and the cell is a microorganism cell. In another exemplary embodiment, the microorganism cell is a bacteria. In another exemplary embodiment, the microorganism cell is a bacteria which is described herein. In another exemplary embodiment, the enzyme is a leucyl tRNA synthetase of a bacteria described herein. In another exemplary embodiment, the tRNA synthetase is leucyl tRNA synthetase. In another exemplary embodiment, the compound has a K_{D, synthesis} of greater than 100 μM against a synthetic domain of said tRNA synthetase.

In certain embodiments, the mechanism of action of a compound of the invention is to inhibit the conversion of a tRNA molecule into a charged tRNA molecule by binding to and/or inhibiting at least the editing domain of the synthetase. The compounds of use in this method may also inhibit or otherwise interact with the synthetic domain (e.g., the active site of the synthetic domain). In a presently preferred embodiment, the editing domain is inhibited selectively in the presence of the synthetic domain. In a preferred embodiment, the synthetic domain is essentially uninhibited, while the editing domain is inhibited at least 50%, preferably at least 60%, more preferably at least 70%, still more preferably, at least 80% and even still more preferably at least 90% of the activity of the tRNA synthetase. In another preferred embodiment, the synthetic domain is inhibited by at most 50%, preferably at most 30%, preferably at most 20%, 10%, preferably at most 8%, more preferably at most 5%, still more preferably, at most 3% and even still more preferably at most 1%. Inhibition of the editing domain produces a decrease in the amount of the properly charged tRNA which results in retardation or cessation of cell growth and division.

In another exemplary embodiment, the ratio of a minimum concentration of said compound inhibiting said editing domain to a minimum concentration of said compound inhibiting said synthetic domain of said tRNA synthetase, represented as K_{D, edit}/K_{D, synthesis}, is less than one. In another exemplary embodiment, the K_{D, edit}/K_{D, synthesis} of the compound is a member selected from less than 0.5, less than 0.1 and less than 0.05.

### VI. c) Inhibiting a Phosphodiesterase

In another aspect, the invention provides a method for inhibiting a phosphodiesterase (PDE), the method comprising: contacting the phosphodiesterase with a compound of the invention, wherein the phosphodiesterase is inhibited. In an exemplary embodiment, the amount of the compound is a therapeutically effective amount. In an exemplary embodiment, the compound of the invention is a compound described herein, or a pharmaceutically acceptable salt thereof. In an exemplary embodiment, the compound of the invention a compound described in a formula provided herein. In an exemplary embodiment, the compound of the invention is a compound described herein.

In an exemplary embodiment, the phosphodiesterase is a member selected from PDE1, PDE2, PDE3, PDE4, PDE5, PDE6, PDE7, PDE8, PDE9, PDE10 and PDE11. In an exemplary embodiment, the phosphodiesterase is PDE4. In an exemplary embodiment, the PDE4 is a member selected from PDE4A, PDE4B, PDE4C and PDE4D. In an exemplary embodiment, the PDE4 is PDE4B. In an exemplary embodiment, the phosphodiesterase is PDE7.

In an exemplary embodiment, the invention provides a method for inhibiting a phosphodiesterase4 (PDE4), but not significantly inhibiting at least one PDE which is a member selected from PDE1, PDE2, PDE3, PDE5 and PDE6, involving contacting a cell with a compound of the invention, thereby providing said inhibition. In an exemplary embodiment, the compound of the invention is a compound described herein, or a pharmaceutically acceptable salt thereof. In an exemplary embodiment, the compound of the invention a compound described in a formula provided herein. In an exemplary embodiment, the compound of the invention is a compound described herein.

In an exemplary embodiment, for any of the methods described herein, the compound of the invention, is present in an amount which will inhibit a phosphodiesterase described herein by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%.

VI. d) Decreasing the Production of a Cytokine and/or Chemokine

In another aspect, the invention provides a method for decreasing the production of a cytokine and/or a chemokine, the method comprising: contacting a cell with a compound of the invention, wherein production of the cytokine and/or chemokine by the cell is decreased. In an exemplary embodiment, the cell is contacted with a therapeutically effective amount of the compound. In an exemplary embodiment, the compound of the invention is a compound described herein, or a pharmaceutically acceptable salt thereof. In an exemplary embodiment, the compound of the invention a compound described in a formula provided herein. In an exemplary embodiment, the compound of the invention is a compound described herein.

In an exemplary embodiment, the method is for decreasing the production of a cytokine, which is a TH1 cytokine In an exemplary embodiment, the TH1 cytokine is a member selected from IFN-g and IL-2.

In an exemplary embodiment, the method is for decreasing the production of a cytokine, which is a TH2 cytokine In an exemplary embodiment, the TH2 cytokine is a member selected from IL-4, IL-5 and IL-10.

In an exemplary embodiment, the method is for decreasing the production of a cytokine, which is a member selected from IL-1α, IL-1β, IL-2, IL-3, IL-6, IL-7, IL-9, IL-12, IL-17, IL-18, IL-23, TNF-α, LT, LIF, Oncostatin, IFNα, IFNβ and IFNγ. In another exemplary embodiment, the cytokine is a member selected from IL-1β, IL-2, IL-3, IL-6, IL-7, IL-9, IL-12, IL-23, TNF-α, LT, LIF, Oncostatin, and IFNγ. In another exemplary embodiment, the cytokine is a member selected from IL-1β, IL-2, IL-23, TNF-α and IFNγ. In another exemplary embodiment, the cytokine is TNF-α.

In an exemplary embodiment, the method is for decreasing the release of a cytokine, which is a member selected from IL-1β, IL-2, IL-4, IL-5, IL-6, IL-8, IL-10, IL-12, IL-23, TNF-α and IFNγ.

In an exemplary embodiment, the method is for decreasing the production of a cytokine, which is a member selected from IL-4, IL-10, IL-11, W-13 and TGF-β.

In an exemplary embodiment, the method is for decreasing the production of a chemokine, which is a member selected from IL-8, Gro-α, MIP-1, MCP-1, PGE2, ENA-78, and RANTES. In an exemplary embodiment, the chemokine is a member selected from MCP-1 and PGE2.

In an exemplary embodiment, for any of the methods described herein, the compound of the invention is present in an amount which will inhibit the production of a cytokine and/or a chemokine by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%.

VI. e) Increasing the Production of a Cytokine and/or a Chemokine

In another aspect, the invention provides a method for increasing the production of a cytokine and/or a chemokine, the method comprising: contacting a cell with a compound of the invention, wherein production of the cytokine and/or chemokine by the cell is increased. In an exemplary embodiment, the cell is contacted with a therapeutically effective amount of the compound. In an exemplary embodiment, the compound of the invention is a compound described herein, or a pharmaceutically acceptable salt thereof. In an exemplary embodiment, the compound of the invention a compound described in a formula provided herein. In an exemplary embodiment, the compound of the invention is a compound described herein.

In an exemplary embodiment, the method is for increasing the production of a cytokine, which is a TH1 cytokine In an exemplary embodiment, the TH1 cytokine is a member selected from IHN-g and IL-2.

In an exemplary embodiment, the method is for increasing the production of a cytokine, which is a TH2 cytokine In an exemplary embodiment, the TH2 cytokine is a member selected from IL-4, IL-5 and IL-10.

In an exemplary embodiment, the method is for increasing the production of a cytokine, which is a member selected from IL-4, IL-10, IL-11, W-13 and TGF-β.

In an exemplary embodiment, the method is for increasing the production of a chemokine, which is a member selected from IL-8, Gro-α, MIP-1, MCP-1, PGE2, ENA-78, and RANTES. In an exemplary embodiment, the chemokine is a member selected from MCP-1 and PGE2.

In an exemplary embodiment, for any of the methods described herein, the of the invention is present in an amount which will increase the production of a cytokine and/or a chemokine by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%.

VI. f) Decreasing the Release of a Cytokine and/or Chemokine

In another aspect, the invention provides a method for decreasing the release of a cytokine and/or a chemokine, the method comprising: contacting a cell with a compound of the invention, wherein the release of the cytokine and/or chemokine by the cell is decreased. In an exemplary embodiment, the cell is contacted with a therapeutically effective amount of the compound. In an exemplary embodiment, the compound of the invention is a compound described herein, or a pharmaceutically acceptable salt thereof. In an exemplary embodiment, the compound of the invention a compound described in a formula provided herein. In an exemplary embodiment, the compound of the invention is a compound described herein.

In an exemplary embodiment, the method is for decreasing the release of a cytokine, which is a TH1 cytokine In an exemplary embodiment, the TH1 cytokine is a member selected from IHN-g and IL-2.

In an exemplary embodiment, the method is for decreasing the release of a cytokine, which is a TH2 cytokine In an exemplary embodiment, the TH2 cytokine is a member selected from IL-4, IL-5 and IL-10.

In an exemplary embodiment, the method is for decreasing the release of a cytokine, which is a member selected from IL-1α, IL-1β, IL-2, IL-3, IL-6, IL-7, IL-9, IL-12, IL-17, IL-18, IL-23, TNF-α, LT, LIF, Oncostatin, IFNα, IFNβ and IFNγ. In another exemplary embodiment, the cytokine is a member selected from IL-1β, IL-2, IL-3, IL-6, IL-7, IL-9, IL-12, IL-23, TNF-α, LT, LIF, Oncostatin, and IFNγ. In another exemplary embodiment, the cytokine is a member selected from IL-1β, IL-2, IL-23, TNF-α and IFNγ. In another exemplary embodiment, the cytokine is TNF-α.

In an exemplary embodiment, the method is for decreasing the release of a cytokine, which is a member selected from IL-1β, IL-2, IL-4, IL-5, IL-6, IL-8, IL-10, IL-12, IL-23, TNF-α and IFNγ.

In an exemplary embodiment, the compound described herein decreases the release of IL-1β, IL-2, IL-4, IL-5, IL-6, IL-8, IL-10, IL-12, IL-23, TNF-α and IFNγ.

In an exemplary embodiment, the method is for decreasing the release of a cytokine, which is a member selected from IL-4, IL-10, IL-11, W-13 and TGF-β.

In an exemplary embodiment, the method is for decreasing the release of a chemokine, which is a member selected from IL-8, Gro-α, MIP-1, MCP-1, PGE2, ENA-78, and RANTES. In an exemplary embodiment, the chemokine is a member selected from MCP-1 and PGE2.

In an exemplary embodiment, the compound described herein decreases the release of TNF-α, IL-2, IFNγ, IL-5, and IL-10, and does not substantially decrease the release of IL-1β, IL-6 and IL-8. In an exemplary embodiment, the compound decreases the release of IL-12 and IL-23.

In an exemplary embodiment, for any of the methods described herein, the compound of the invention is present in an amount which will decrease the release of a cytokine and/or a chemokine by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%.

VI. g) Increasing the Release of a Cytokine and/or a Chemokine

In another aspect, the invention provides a method for increasing the production of a cytokine and/or a chemokine, the method comprising: contacting a cell with a compound of the invention, wherein release of the cytokine and/or chemokine by the cell is increased. In an exemplary embodiment, the cell is contacted with a therapeutically effective amount of the compound. In an exemplary embodiment, the compound of the invention is a compound described herein, or a pharmaceutically acceptable salt thereof. In an exemplary embodiment, the compound of the invention a compound described in a formula provided herein. In an exemplary embodiment, the compound of the invention is a compound described herein.

In an exemplary embodiment, the method is for increasing the release of a cytokine, which is a TH1 cytokine In an exemplary embodiment, the TH1 cytokine is a member selected from IFN-γ and IL-2.

In an exemplary embodiment, the method is for increasing the release of a cytokine, which is a TH2 cytokine In an exemplary embodiment, the TH2 cytokine is a member selected from IL-4, IL-5 and IL-10.

In an exemplary embodiment, the method is for increasing the release of a cytokine, which is a member selected from IL-4, IL-10, IL-11, W-13 and TGF-β.

In an exemplary embodiment, the method is for increasing the release of a chemokine, which is a member selected from IL-8, Gro-α, MIP-1, MCP-1, PGE2, ENA-78, and RANTES. In an exemplary embodiment, the chemokine is a member selected from MCP-1 and PGE2.

In an exemplary embodiment, for any of the methods described herein, the compound of the invention is present in an amount which will increase release of a cytokine and/or a chemokine by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%.

### VI. h) Inhibiting Microorganism Growth or Killing Microorganisms

The compounds of the present invention and/or combinations of the invention exhibit potency against microorganisms, such as bacteria, and therefore have the potential to kill and/or inhibit the growth of microorganisms. Testing for the presence of a beta-lactamase in a bacteria can be accomplished using methods known to one of skill in the art. See, for example, Sturenburg et al., J. Antimic. Chemo., (2004) 54, 134-138 and Tan et al, Antimicrob. Agents Chemother., (2009) 53(1): 146-149.

In a further aspect, the invention provides a method of killing and/or inhibiting the growth of a microorganism, said method comprising: contacting said microorganism with an effective amount of a compound of the invention, thereby killing and/or inhibiting the growth of the microorganism. In a further aspect, the invention provides a method of killing and/or inhibiting the growth of a microorganism, said method comprising: contacting said microorganism with an effective amount of a combination of the invention, thereby killing and/or inhibiting the growth of the microorganism. In an exemplary embodiment, the microorganism is a bacteria. In an exemplary embodiment, the compound is described herein, or a salt, prodrug, hydrate or solvate thereof, or a combination thereof. In an exemplary embodiment, the invention provides a compound described herein, or a salt, hydrate or solvate thereof. In an exemplary embodiment, the invention provides a compound described herein, or a prodrug thereof. In an exemplary embodiment, the invention provides a compound described herein, or a salt thereof. In another exemplary embodiment, the compound of the invention is a compound described herein, or a pharmaceutically acceptable salt thereof. In another exemplary embodiment, the compound is described by a formula listed herein, or a pharmaceutically acceptable salt thereof. In an exemplary embodiment, the compound is part of a combination described herein. In an exemplary embodiment, the compound is part of a pharmaceutical formulation described herein. In another exemplary embodiment, the contacting occurs under conditions which permit entry of the compound into the organism. Such conditions are known to one skilled in the art and are described herein.

In another aspect, the microorganism is inside, or on the surface of an animal. In an exemplary embodiment, the animal is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human.

In an exemplary embodiment, the microorganism is killed or its growth is inhibited through oral administration of the compound of the invention and/or the combination of the invention. In an exemplary embodiment, the microorganism is killed or its growth is inhibited through intravenous administration of the compound of the invention and/or the combination of the invention.

In an exemplary embodiment, the microorganism is a bacterium. In an exemplary embodiment, the bacterium is a gram-positive bacteria. In another exemplary embodiment, the gram-positive bacterium is a member selected from Staphylococcus species, Streptococcus species, Bacillus species, Mycobacterium species, Corynebacterium species (Propionibacterium species), Clostridium species, Actinomyces species, Enterococcus species and Streptomyces species. In another exemplary embodiment, the gram-positive bacterium is a member selected from Propionibacterium acnes; Staphylococcus aureus; Staphylococcus epidermidis, Staphylococcus saprophyticus; Staphylococcus haemolyticus; Streptococcus pyogenes; Streptococcus agalactiae; Streptococcus pneumoniae; Enterococcus faecalis; Enterococcus faecium; Bacillus anthracis; Mycobacterium avium-intracellulare; Mycobacterium tuberculosis, Acinetobacter baumanii; Corynebacterium diphtheria; Clostridium perfringens; Clostridium botulinum; Clostridium tetani; Clostridium difficile. In another exemplary embodiment, the gram-positive bacterium is a member selected from Staphylococcus aureus, Staphylococcus epidermidis, Streptococcus pneumoniae, Streptococcus pyogenes, Enterococcus faecalis, Enterococcus faecium, Clostridium difficile and Propionibacter acnes. In another exemplary embodiment, the bacterium is a gram-negative bacterium. In another exemplary embodiment, the gram-negative bacterium is a member selected from Acinetobacter species, Neisseria species, Pseudomonas species, Brucella species, Agrobacterium species, Bordetella species, Escherichia species, Shigelia species, Yersinia species, Salmonella species, Klebsiella species, Enterobacter species, Haemophilus species, Pasteurella species, Streptobacillus species, spirochetal species, Campylobacter species, Vibrio species, Helicobacter species, Bacteroides species, Citrobacter species, Proteus species, Providencia species, Serratia species, Stenotrophomonas species and Burkholderia species. In another exemplary embodiment, the gram-negative bacterium is a member selected from Acinetobacter species, Pseudomonas species, Escherichia species, Klebsiella species, Enterobacter species, Bacteroides species, Citrobacter species, Proteus species, Providencia species, Serratia species, Stenotrophomonas species and Burkholderia species. In another exemplary embodiment, the gram-negative bacterium is a member selected from Neisseria gonorrhoeae; Neisseria meningitidis; Pseudomonas aeruginosa; Legionella pneumophila; Escherichia coli; Yersinia pestis; Haemophilus influenzae; Helicobacter pylori; Campylobacter fetus; Campylobacter jejuni; Vibrio cholerae; Vibrio parahemolyticus; Trepomena pallidum; Actinomyces israelii; Rickettsia prowazekii; Rickettsia rickettsii; Chlamydia trachomatis; Chlamydia psittaci; Brucella abortus; Agrobacterium tumefaciens; Francisella tularensis, Klebsiella pneumoniae, Enterobacter cloacae, Acinetobacter baumannii, Bacteroides fragilis, Citrobacter freundii, Proteus mirabilis, Providencia stuartii, Serratia marcescens, Stenotrophomonas maltophilia and Burkholderia cepacia. In another exemplary embodiment, the gram-negative bacterium is a member selected from Pseudomonas aeruginosa; Escherichia coli; Haemophilus influenzae, Klebsiella pneumoniae, Enterobacter cloacae, Acinetobacter baumannii, Bacteroides fragilis, Citrobacter freundii, Proteus mirabilis, Providencia stuartii, Serratia marcescens, Stenotrophomonas maltophilia and Burkholderia cepacia. In another exemplary embodiment, the gram-negative bacterium is a member selected from Enterobacter aerogenes; Enterobacter cloacae; Enterobacter sakazakii; Escherichia coli; Klebsiella pneumoniae; Proteus mirabilis; Serratia marcescens and Citrobacter freundii. In another exemplary embodiment, the gram-negative bacterium is a Providencia spp. In another exemplary embodiment, the gram-negative bacterium is an Enterobacter spp.

In another exemplary embodiment, the bacterium is a Pseudomonas species. In another exemplary embodiment, the bacterium is Pseudomonas aeruginosa. In another exemplary embodiment, the bacterium is a member selected from Pseudomonas aeruginosa; Acinetobacter baumannii, Stenotrophomonas maltophilia and Burkholderia cepacia. In another exemplary embodiment, the bacterium is Acinetobacter baumannii. In another exemplary embodiment, the bacterium is Stenotrophomonas maltophilia. In another exemplary embodiment, the bacterium is Burkholderia cepacia. In another exemplary embodiment, the bacterium is Acinetobacter species. In another exemplary embodiment, the bacterium is Acinetobacter anitratus. In another exemplary embodiment, the bacterium is a member selected from Enterobacter aerogenes, Enterobacter cloacae, Enterobacter sakazakii, E. coli, K. pneumoniae, P. mirabilis, Serratia marcescens, Citrobacter freundii and Providencia spp. In another exemplary embodiment, the bacterium is a member selected from Enterobacter aerogenes, Enterobacter cloacae, Enterobacter sakazakii, E. coli, K. pneumoniae, P. mirabilis, Serratia marcescens, Citrobacter freundii, Providencia spp., S. aureus, S. pneumonia, S. pyogenes, E. faecalis, and E. faecium. In another exemplary embodiment, the bacterium is a member selected from Pseudomonas aeruginosa; Acinetobacter baumannii; Stenotrophomonas maltophilia; Burkholderia cepacia. In another exemplary embodiment, the bacterium is a member selected from S. aureus, S. pneumonia, S. pyogenes, E. faecalis, and E. faecium. In another exemplary embodiment, the bacterium is a member selected from Viridans group Strep. In another exemplary embodiment, the bacterium is a member selected from Strep. mitis, Strep. mutans, Strep. oxalis, Strep. sanguis, Strep. sobrinus and Strep. millari. In another exemplary embodiment, the bacterium is S. pneumonia. In another exemplary embodiment, the bacterium is H. influenzae. In another exemplary embodiment, the bacterium is S. aureus. In another exemplary embodiment, the bacterium is M. catarrhalis. In another exemplary embodiment, the bacterium is M. pneumoniae. In another exemplary embodiment, the bacterium is L. pneumoniae. In another exemplary embodiment, the bacterium is C. pneumoniae. In another exemplary embodiment, the bacterium is S. pyogenes. In another exemplary embodiment, the bacterium is an anaerobe. In another exemplary embodiment, the bacterium is an Alcaligenes species. In another exemplary embodiment, the bacterium is a B. cepacia. In another exemplary embodiment, the bacterium is a member selected from Enterobacter cloacae, Escherichia coli; Klebsiella pneumoniae, Proteus mirabilis, Providencia stuartii, Serratia marcescens, and Citrobacter freundii. In another exemplary embodiment, the bacterium is resistant to methicillin. In another exemplary embodiment, the bacterium is methicillin-resistant staphylococcus aureus. In another exemplary embodiment, the bacterium is a member selected from Streptococcus pneumoniae; Haemophilus influenzae; Staphylococcus aureus; Mycobacterium catarrhalis; Mycobacterium pneumoniae; Legionella pneumophila and Chlamydia pneumoniae. In another exemplary embodiment, the bacterium is a member selected from Enterobacter cloacae, Escherichia coli; Klebsiella pneumoniae, Proteus mirabilis, Serratia marcescens, Citrobacter freundii, Providencia stuartii, Pseudomonas aeruginosa; Acinetobacter baumannii, Stenotrophomonas maltophilia, Burkholderia cepacia, Staphylococcus aureus; Streptococcus pneumoniae; Streptococcus pyogenes; Enterococcus faecalis; and Enterococcus faecium. In another exemplary embodiment, the bacterium is a member selected from Staphylococcus aureus; Staphylococcus epidermidis, Staphylococcus haemolyticus; Streptococcus pyogenes; Streptococcus agalactiae and Streptococcus pneumoniae.

In an exemplary embodiment, the microorganism is a bacterium, which is a member selected from acid-fast bacteria, including Mycobacterium species; bacilli, including Bacillus species, Corynebacterium species (also Propionibacterium) and Clostridium species; filamentous bacteria, including Actinomyces species and Streptomyces species; bacilli, such as Pseudomonas species, Brucella species, Agrobacterium species, Bordetella species, Escherichia species, Shigella species, Yersinia species, Salmonella species, Klebsiella species, Enterobacter species, Haemophilus species, Pasteurella species, and Streptobacillus species; spirochetal species, Campylobacter species, Vibrio species; and intracellular bacteria including Rickettsiae species and Chlamydia species.

### VI. i) Microorganism Infection

The compounds of the present invention and/or combinations of the invention exhibit potency against microorganisms, such as bacteria, and therefore have the potential to be used to treat and/or prevent a microorganism infection, such as a bacterial infection.

In a further aspect, the invention provides a method of treating a bacterial infection comprising administering to an animal suffering from the infection an effective amount of a compound of the invention, or a pharmaceutically acceptable salt thereof, thereby treating the bacterial infection. In an exemplary embodiment, the invention provides a method of treating a bacterial infection comprising administering to an animal suffering from the infection an effective amount of a compound of the invention, or a pharmaceutically acceptable salt thereof, and an effective amount of an antibiotic, or a pharmaceutically acceptable salt thereof, thereby treating the bacterial infection.

In a further aspect, the invention provides a method of preventing a bacterial infection comprising administering to an animal a prophylactic amount of a compound of the invention, or a pharmaceutically acceptable salt thereof, thereby treating the bacterial infection. In an exemplary embodiment, the invention provides a method of preventing a bacterial infection comprising administering to an animal a prophylactic amount of a compound of the invention, or a pharmaceutically acceptable salt thereof, and an effective amount of an antibiotic, or a pharmaceutically acceptable salt thereof, thereby treating the bacterial infection.

In an exemplary embodiment, the compound used in the method is described herein, or a salt, prodrug, hydrate or solvate thereof, or a combination thereof. In an exemplary embodiment, the compound used in the method is described herein, or a salt, hydrate or solvate thereof. In an exemplary embodiment, compound used in the method is described herein, or a prodrug thereof. In an exemplary embodiment, the compound used in the method is described herein, or a salt thereof. In another exemplary embodiment, the compound of the invention is a compound described herein, or a pharmaceutically acceptable salt thereof. In another exemplary embodiment, the compound is described by a formula listed herein, or a pharmaceutically acceptable salt thereof. In an exemplary embodiment, the compound is part of a combination described herein. In an exemplary embodiment, the compound is part of a pharmaceutical formulation described herein. In another exemplary embodiment, the administering occurs under conditions which permit entry of the compound into the animal, and subsequently into the bacteria. Such conditions are known to one skilled in the art and specific conditions are set forth herein.

In another aspect, the microorganism is inside, or on the surface of an animal. In an exemplary embodiment, the animal is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human.

In an exemplary embodiment, the bacterial infection is treated and/or prevented through oral administration of the compound of the invention and/or the combination of the invention. In an exemplary embodiment, the bacterial infection is treated and/or prevented through intravenous administration of the compound of the invention and/or the combination of the invention. In an exemplary embodiment, the bacterial infection is treated and/or prevented through topical administration of the compound of the invention and/or the combination of the invention.

In an exemplary embodiment, the bacterial infection is caused by and/or associated with a gram-positive bacteria. In another exemplary embodiment, the gram-positive bacterium is a member selected from Staphylococcus species, Streptococcus species, Bacillus species, Mycobacterium species, Corynebacterium species (Propionibacterium species), Clostridium species, Actinomyces species, Enterococcus species and Streptomyces species. In another exemplary embodiment, the gram-positive bacterium is a member selected from Propionibacterium acnes; Staphylococcus aureus; Staphylococcus epidermidis, Staphylococcus saprophyticus; Staphylococcus haemolyticus; Streptococcus pyogenes; Streptococcus agalactiae; Streptococcus pneumoniae; Enterococcus faecalis; Enterococcus faecium; Bacillus anthracis; Mycobacterium avium-intracellulare; Mycobacterium tuberculosis, Acinetobacter baumanii; Corynebacterium diphtheria; Clostridium perfringens; Clostridium botulinum; Clostridium tetani; Clostridium difficile. In another exemplary embodiment, the gram-positive bacterium is a member selected from Staphylococcus aureus, Staphylococcus epidermidis, Streptococcus pneumoniae, Streptococcus pyogenes, Enterococcus faecalis, Enterococcus faecium, Clostridium difficile and Propionibacter acnes.

In an exemplary embodiment, the bacterial infection is caused by and/or associated with a gram-negative bacterium. In another exemplary embodiment, the gram-negative bacterium is a member selected from Acinetobacter species, Neisseria species, Pseudomonas species, Brucella species, Agrobacterium species, Bordetella species, Escherichia species, Shigelia species, Yersinia species, Salmonella species, Klebsiella species, Enterobacter species, Haemophilus species, Pasteurella species, Streptobacillus species, spirochetal species, Campylobacter species, Vibrio species, Helicobacter species, Bacteroides species, Citrobacter species, Proteus species, Providencia species, Serratia species, Stenotrophomonas species and Burkholderia species. In another exemplary embodiment, the gram-negative bacterium is a member selected from Acinetobacter species, Pseudomonas species, Escherichia species, Klebsiella species, Enterobacter species, Bacteroides species, Citrobacter species, Proteus species, Providencia species, Serratia species, Stenotrophomonas species and Burkholderia species. In another exemplary embodiment, the gram-negative bacterium is a member selected from Neisseria gonorrhoeae; Neisseria meningitidis; Pseudomonas aeruginosa; Legionella pneumophila; Escherichia coli; Yersinia pestis; Haemophilus influenzae; Helicobacter pylori; Campylobacter fetus; Campylobacter jejuni; Vibrio cholerae; Vibrio parahemolyticus; Trepomena pallidum; Actinomyces israelii; Rickettsia prowazekii; Rickettsia rickettsii; Chlamydia trachomatis; Chlamydia psittaci; Brucella abortus; Agrobacterium tumefaciens; Francisella tularensis, Klebsiella pneumoniae, Enterobacter cloacae, Acinetobacter baumannii, Bacteroides fragilis, Citrobacter freundii, Proteus mirabilis, Providencia stuartii, Serratia marcescens, Stenotrophomonas maltophilia and Burkholderia cepacia. In another exemplary embodiment, the gram-negative bacterium is a member selected from Pseudomonas aeruginosa; Escherichia coli; Haemophilus influenzae, Klebsiella pneumoniae, Enterobacter cloacae, Acinetobacter baumannii, Bacteroides fragilis, Citrobacter freundii, Proteus mirabilis, Providencia stuartii, Serratia marcescens, Stenotrophomonas maltophilia and Burkholderia cepacia. In another exemplary embodiment, the gram-negative bacterium is a member selected from Enterobacter aerogenes; Enterobacter cloacae; Enterobacter sakazakii; Escherichia coli; Klebsiella pneumoniae; Proteus mirabilis; Serratia marcescens and Citrobacter freundii. In another exemplary embodiment, the gram-negative bacterium is a Providencia spp. In another exemplary embodiment, the gram-negative bacterium is an Enterobacter spp.

In another exemplary embodiment, the bacterial infection is caused by and/or associated with a Pseudomonas species. In another exemplary embodiment, the bacterial infection is caused by and/or associated with Pseudomonas aeruginosa. In another exemplary embodiment, the bacterial infection is caused by and/or associated with a member selected from Pseudomonas aeruginosa; Acinetobacter baumannii, Stenotrophomonas maltophilia and Burkholderia cepacia. In another exemplary embodiment, the bacterial infection is caused by and/or associated with Acinetobacter baumannii. In another exemplary embodiment, the bacterial infection is caused by and/or associated with Stenotrophomonas maltophilia. In another exemplary embodiment, the bacterial infection is caused by and/or associated with Burkholderia cepacia. In another exemplary embodiment, the bacterial infection is caused by and/or associated with Acinetobacter species. In another exemplary embodiment, the bacterial infection is caused by and/or associated with Acinetobacter anitratus. In another exemplary embodiment, the bacterial infection is caused by and/or associated with a member selected from Enterobacter aerogenes, Enterobacter cloacae, Enterobacter sakazakii, E. coli, K. pneumoniae, P. mirabilis, Serratia marcescens, Citrobacter freundii and Providencia spp. In another exemplary embodiment, the bacterial infection is caused by and/or associated with a member selected from Enterobacter aerogenes, Enterobacter cloacae, Enterobacter sakazakii, E. coli, K. pneumoniae, P. mirabilis, Serratia marcescens, Citrobacter freundii, Providencia spp., S. aureus, S. pneumonia, S. pyogenes, E. faecalis, and E. faecium. In another exemplary embodiment, the bacterial infection is caused by and/or associated with a member selected from Pseudomonas aeruginosa; Acinetobacter baumannii; Stenotrophomonas maltophilia; Burkholderia cepacia. In another exemplary embodiment, the bacterial infection is caused by and/or associated with a member selected from S. aureus, S. pneumonia, S. pyogenes, E. faecalis, and E. faecium. In another exemplary embodiment, the bacterial infection is caused by and/or associated with Viridans group Strep. In another exemplary embodiment, the bacterial infection is caused by and/or associated with a member selected from Strep. mitis, Strep. mutans, Strep. oxalis, Strep. sanguis, Strep. sobrinus and Strep. millari. In another exemplary embodiment, the bacterial infection is caused by and/or associated with S. pneumonia. In another exemplary embodiment, the bacterial infection is caused by and/or associated with H. influenzae. In another exemplary embodiment, the bacterial infection is caused by and/or associated with S. aureus. In another exemplary embodiment, the bacterial infection is caused by and/or associated with M. catarrhalis. In another exemplary embodiment, the bacterial infection is caused by and/or associated with M. pneumoniae. In another exemplary embodiment, the bacterial infection is caused by and/or associated with L. pneumoniae. In another exemplary embodiment, the bacterial infection is caused by and/or associated with C. pneumoniae. In another exemplary embodiment, the bacterial infection is caused by and/or associated with S. pyogenes. In another exemplary embodiment, the bacterial infection is caused by and/or associated with an anaerobe. In another exemplary embodiment, the bacterial infection is caused by and/or associated with Alcaligenes species. In another exemplary embodiment, the bacterial infection is caused by and/or associated with B. cepacia. In another exemplary embodiment, the bacterial infection is caused by and/or associated with a member selected from Enterobacter cloacae, Escherichia coli; Klebsiella pneumoniae, Proteus mirabilis, Providencia stuartii, Serratia marcescens, and Citrobacter freundii. In another exemplary embodiment, the bacterial infection is caused by and/or associated with a bacteria which is resistant to methicillin. In another exemplary embodiment, the bacterial infection is caused by and/or associated with methicillin-resistant staphylococcus aureus. In another exemplary embodiment, the bacterial infection is caused by and/or associated with a member selected from Streptococcus pneumoniae; Haemophilus influenzae; Staphylococcus aureus; Mycobacterium catarrhalis; Mycobacterium pneumoniae; Legionella pneumophila and Chlamydia pneumoniae. In another exemplary embodiment, the bacterial infection is caused by and/or associated with a member selected from Enterobacter cloacae, Escherichia coli; Klebsiella pneumoniae, Proteus mirabilis, Serratia marcescens, Citrobacter freundii, Providencia stuartii, Pseudomonas aeruginosa; Acinetobacter baumannii, Stenotrophomonas maltophilia, Burkholderia cepacia, Staphylococcus aureus; Streptococcus pneumoniae; Streptococcus pyogenes; Enterococcus faecalis; and Enterococcus faecium. In another exemplary embodiment, the bacterial infection is caused by and/or associated with a member selected from Staphylococcus aureus; Staphylococcus epidermidis, Staphylococcus haemolyticus; Streptococcus pyogenes; Streptococcus agalactiae and Streptococcus pneumoniae.

In an exemplary embodiment, the bacterial infection is caused by and/or associated with a member selected from acid-fast bacteria, including Mycobacterium species; bacilli, including Bacillus species, Corynebacterium species (also Propionibacterium) and Clostridium species; filamentous bacteria, including Actinomyces species and Streptomyces species; bacilli, such as Pseudomonas species, Brucella species, Agrobacterium species, Bordetella species, Escherichia species, Shigella species, Yersinia species, Salmonella species, Klebsiella species, Enterobacter species, Haemophilus species, Pasteurella species, and Streptobacillus species; spirochetal species, Campylobacter species, Vibrio species; and intracellular bacteria including Rickettsiae species and Chlamydia species.

### VI. j) Diseases

The compounds of the invention and/or combinations of the present invention exhibit potency against microorganisms, such as bacteria, and therefore have the potential to achieve therapeutic efficacy in the animals described herein.

In another aspect, the invention provides a method of treating and/or preventing a disease. In an exemplary embodiment, the method includes administering to the animal a therapeutically effective amount of a compound of the invention, sufficient to treat and/or prevent the disease. In an exemplary embodiment, the method includes administering to the animal a therapeutically effective amount of a combination of the invention, sufficient to treat and/or prevent the disease. In an exemplary embodiment, the compound of the invention or the combination of the invention can be used in human or veterinary medical therapy, particularly in the treatment or prophylaxis of bacterial-associated disease. In an exemplary embodiment, the compound is described herein, or a salt, prodrug, hydrate or solvate thereof, or a combination thereof. In an exemplary embodiment, the invention provides a compound described herein, or a prodrug thereof. In an exemplary embodiment, the invention provides a compound described herein, or a salt, hydrate or solvate thereof. In an exemplary embodiment, the invention provides a compound described herein, or a salt thereof. In another exemplary embodiment, the compound of the invention is a compound described herein, or a pharmaceutically acceptable salt thereof. In an exemplary embodiment, the compound is a compound described herein, or a pharmaceutically acceptable salt thereof. In an exemplary embodiment, the compound is according to a formula described herein, or a pharmaceutically acceptable salt thereof. In an exemplary embodiment, the compound is part of a combination described herein. In an exemplary embodiment, the compound is part of a pharmaceutical formulation described herein. In another exemplary embodiment, the animal is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human. In another exemplary embodiment, the animal is a member selected from a human, cattle, goat, pig, sheep, horse, cow, bull, dog, guinea pig, gerbil, rabbit, cat, chicken and turkey. In another exemplary embodiment, the disease is a member selected from a systemic disease. In another exemplary embodiment, the disease is a topical disease.

In an exemplary embodiment, the disease is treated through oral administration of a compound of the invention and/or a combination of the invention. In an exemplary embodiment, the disease is treated through intravenous administration of a compound of the invention and/or a combination of the invention.

Systemic Diseases

In another aspect, the invention provides a method of treating a systemic disease. The method involves contacting an animal with a compound of the invention and/or a combination of the invention.

In an exemplary embodiment, the disease is a member selected from candidiasis, aspergillosis, coccidioidomycosis, cryptococcosis, histoplasmosis, blastomycosis, paracoccidioidomycosis, zygomycosis, phaeohyphomycosis and rhinosporidiosis.

In another exemplary embodiment, the disease is associated with infection by a Gram-positive bacteria. In an exemplary embodiment, the disease is associated with a Staphylococcus species. In another exemplary embodiment, the disease is a member selected from pneumonia, gastroenteritis, toxic shock syndrome, community acquired pneumonia (CAP), meningitis, septic arthritis, urinary tract infection, bacteremia, endocarditis, osteomylitis, skin and skin-structure infection. In an exemplary embodiment, the disease is associated with a Streptococcus species. In another exemplary embodiment, the disease is a member selected from strep throat, skin infections, necrotizing fasciitis, toxic shock syndrome, pneumonia, otitis media and sinusitis. In an exemplary embodiment, the disease is associated with an Actinomyces species. In another exemplary embodiment, the disease is actinomycosis. In an exemplary embodiment, the disease is associated with a Norcardia species. In another exemplary embodiment, the disease is pneumonia. In an exemplary embodiment, the disease is associated with a Corynebacterium species. In another exemplary embodiment, the disease is diptheria. In an exemplary embodiment, the disease is associated with a Listeria species. In another exemplary embodiment, the disease is meningitis. In an exemplary embodiment, the disease is associated with a Bacillus species. In another exemplary embodiment, the disease is a member selected from anthrax and food poisoning. In an exemplary embodiment, the disease is associated with a Clostridium species. In another exemplary embodiment, the disease is a member selected from botulism, tetanus, gas gangrene and diarrhea. In an exemplary embodiment, the disease is associated with a Mycobacterium species. In another exemplary embodiment, the disease is a member selected from tuberculosis and leprosy.

In another exemplary embodiment, the disease is associated with infection by a Gram-negative bacteria. In an exemplary embodiment, the disease is associated with a Neisseria species. In another exemplary embodiment, the disease is a member selected from meningitis, gonorrhea, otitis extema and folliculitis. In an exemplary embodiment, the disease is associated with an Escherichia species. In another exemplary embodiment, the disease is a member selected from diarrhea, urinary tract infections, meningitis, sepsis and HAP. In an exemplary embodiment, the disease is associated with a Shigella species. In another exemplary embodiment, the disease is a member selected from diarrhea, bacteremia, endocarditis, meningitis and gastroenteritis. In an exemplary embodiment, the disease is associated with a Salmonella species. In another exemplary embodiment, the disease is a member selected from Typhoid fever, supsis, gastroenteritis, endocarditis, sinusitis and meningitis. In an exemplary embodiment, the disease is associated with a Yersinia species. In another exemplary embodiment, the disease is a member selected from Typhoid fever, bubonic plague, enteric fever and gastroenteritis. In an exemplary embodiment, the disease is associated with a Klebsiella species. In another exemplary embodiment, the disease is a member selected from sepsis and urinary tract infection. In an exemplary embodiment, the disease is associated with a Proteus species. In another exemplary embodiment, the disease is an urinary tract infection. In an exemplary embodiment, the disease is associated with an Enterobacter species. In another exemplary embodiment, the disease is a hospital-acquired infection. In an exemplary embodiment, the disease is associated with a Serratia species. In another exemplary embodiment, the disease is a member selected from a urinary tract infection, skin and skin-structure infection and pneumonia. In an exemplary embodiment, the disease is associated with a Vibrio species. In another exemplary embodiment, the disease is a member selected from cholera and gastroenteritis. In an exemplary embodiment, the disease is associated with a Campylobacter species. In another exemplary embodiment, the disease is gastroenteritis. In an exemplary embodiment, the disease is associated with a Helicobacter species. In another exemplary embodiment, the disease is chronic gastritis. In an exemplary embodiment, the disease is associated with a Pseudomonas species. In another exemplary embodiment, the disease is a member selected from pneumonia, osteomylitis, burn-wound infections, sepsis, UTIs, endocarditis, otitis, corneal infections. In an exemplary embodiment, the disease is associated with a Bacteroides species. In another exemplary embodiment, the disease is a member selected from periodontal disease and aspriation pneumonia. In an exemplary embodiment, the disease is associated with a Haemophilus species. In another exemplary embodiment, the disease is a member selected from meningitis, epiglottitis, septic arthritis, sepsis, chancroid and vaginitis. In an exemplary embodiment, the disease is associated with a Bordetella species. In another exemplary embodiment, the disease is Whooping cough. In an exemplary embodiment, the disease is associated with a Legionella species. In another exemplary embodiment, the disease is a member selected from pneumonia and pontiac fever. In an exemplary embodiment, the disease is associated with a Francisella species. In another exemplary embodiment, the disease is tularemia. In an exemplary embodiment, the disease is associated with a Brucella species. In another exemplary embodiment, the disease is brucellosis. In an exemplary embodiment, the disease is associated with a Pasteurella species. In another exemplary embodiment, the disease is a skin infection. In an exemplary embodiment, the disease is associated with a Gardnerella species. In another exemplary embodiment, the disease is vaginitis. In an exemplary embodiment, the disease is associated with a Spirochetes species. In another exemplary embodiment, the disease is syphilis and Lyme disease. In an exemplary embodiment, the disease is associated with a Chlamydia species. In another exemplary embodiment, the disease is chlamydia. In an exemplary embodiment, the disease is associated with a Rickettsiae species. In another exemplary embodiment, the disease is a member selected from Rocky Mountain spotted fever and typhus.

In an exemplary embodiment, the disease is associated with Mycoplasma pneumoniae. In another exemplary embodiment, the disease is a member selected from tracheobronchitis and walking pneumonia. In an exemplary embodiment, the disease is associated with Ureaplasma urealyticum. In another exemplary embodiment, the disease is urethritis. In another exemplary embodiment, the disease is pyelonephritis. In another exemplary embodiment, the disease is an intra-abdominal infection. In another exemplary embodiment, the disease is febrile neutropenia. In another exemplary embodiment, the disease is a pelvic infection. In another exemplary embodiment, the disease is bacteraemia. In another exemplary embodiment, the disease is septicaemia.

In an exemplary embodiment, the disease is an acute exacerbation of chronic obstructive pulmonary disease. In an exemplary embodiment, the disease is chronic obstructive pulmonary disease. In an exemplary embodiment, the disease is pharyngitis. In an exemplary embodiment, the disease is tonsillitis. In an exemplary embodiment, the disease is Acute Exacerbation of Chronic Bronchitis (AECB). In an exemplary embodiment, the disease is cervicitis. In an exemplary embodiment, the disease is genital ulcer disease.

### VI. k) Conditions and Effects

In another aspect, the invention provides a method of treating and/or preventing a condition, or enhancing an effect, in an animal, the method comprising administering to the animal an amount of a compound of the invention, thereby treating or preventing the condition. In an exemplary embodiment, the amount is a therapeutically effective amount. In an exemplary embodiment, the compound of the invention is a compound described herein, or a pharmaceutically acceptable salt thereof. In an exemplary embodiment, the compound of the invention a compound described in a formula provided herein. In an exemplary embodiment, the compound of the invention is a compound described herein.

In an exemplary embodiment, the condition is a disease. In an exemplary embodiment, the condition is an inflammatory-related condition. In an exemplary embodiment, the condition involves the increase of production of a cytokine and/or a chemokine In an exemplary embodiment, the condition involves the decrease of production of a cytokine and/or a chemokine In an exemplary embodiment, the condition involves the increase of release of a cytokine and/or a chemokine In an exemplary embodiment, the condition involves the decrease of release of a cytokine and/or a chemokine In an exemplary embodiment, the condition involves the inhibition of a phosphodiesterase. In an exemplary embodiment, the compound is in an amount sufficient to treat the inflammatory-related disease by inhibiting pro-inflammatory cytokine expression or by stimulating anti-inflammatory cytokine expression, but the amount is less than sufficient to substantially inhibit cyclin dependent kinases. In an exemplary embodiment, the condition is mediated by a cytokine In an exemplary embodiment, the condition is mediated by a chemokine In an exemplary embodiment, the condition is mediated by a neutrophil. In an exemplary embodiment, the condition is mediated by a phosphodiesterase. In an exemplary embodiment, the condition is mediated by a phosphodiesterase4. In an exemplary embodiment, the condition is mediated by a phosphodiesterase7.

In an exemplary embodiment, the condition is a member selected from periodontitis, dry eye disease, rheumatoid arthritis, osteoarthritis, Crohn's disease, ulcerative colitis, psoriatic arthritis, traumatic arthritis, rubella arthritis, inflammatory bowel disease, multiple sclerosis, psoriasis, graft versus host disease, systemic lupus erythematosus, toxic shock syndrome, irritable bowel syndrome, muscle degeneration, allograft rejections, pancreatitis, insulinitis, glomerulonephritis, diabetic nephropathy, renal fibrosis, chronic renal failure, gout, leprosy, acute synovitis, Reiter's syndrome, gouty arthritis, Behcet's disease, spondylitis, endometriosis, non-articular inflammatory conditions, such as intervertebral disk syndrome conditions, bursitis, tendonitis, tenosynovitis or fibromyalgic syndrome; and acute or chronic pain, including but not limited to neurological pain, neuropathies, polyneuropathies, diabetes-related polyneuropathies, trauma, migraine, tension and cluster headache, Horton's disease, varicose ulcers, neuralgias, musculo-skeletal pain, osteo-traumatic pain, fractures, algodystrophy, spondylarthritis, fibromyalgia, phantom limb pain, back pain, vertebral pain, post-surgery pain, herniated intervertebral disc-induced sciatica, cancer-related pain, vascular pain, visceral pain, childbirth, or HIV-related pain. Other cytokine mediated diseases are allergy, a metabolic disease, a chemotherapy/radiation related complication; diabetes type I; diabetes type II; a liver disease; a gastrointestinal disorder; an ophthamological disease; allergic conjunctivitis; diabetic retinopathy; Sjogren's syndrome; uveitis; a pulmonary disorder, a renal disease; dermatitis; HIV-related cachexia; cerebral malaria; ankylosing spondolytis; leprosy; anemia; fibromyalgia, kidney failure, stroke, chronic heart failure, endotoxemia, reperfusion injury, ischemia reperfusion, myocardial ischemia, restenosis, thrombosis, angiogenesis, Coronary Heart Disease, Coronary Artery Disease, acute coronary syndrome, Takayasu arteritis, cardiac failure such as heart failure, aortic valve stenosis, cardiomyopathy, myocarditis, vasculitis, vascular restenosis, valvular disease or coronary artery bypass; hypercholesteremia, diseases or conditions related to blood coagulation or fibrinolysis, such as for example, acute venous thrombosis, pulmonary embolism, thrombosis during pregnancy, hemorrhagic skin necrosis, acute or chronic disseminated intravascular coagulation (DIC), clot formation from surgery, long bed rest or long periods of immobilization, venous thrombosis, fulminant meningococcemia, acute thrombotic strokes, acute coronary occlusion, acute peripheral arterial occlusion, massive pulmonary embolism, axillary vein thrombosis, massive iliofemoral vein thrombosis, occluded arterial or venous cannulae, cardiomyopathy, venoocclusive disease of the liver, hypotension, decreased cardiac output, decreased vascular resistance, pulmonary hypertension, diminished lung compliance, leukopenia or thrombocytopenia; or atherosclerosis.

In an exemplary embodiment, the condition is a member selected from allergic conjunctivitis, uveitis, glaucoma, optic neuritis, retinal ischemia, diabetic retinopathy, laser induced optic damage, or surgery or trauma-induced proliferative vitreoretinopathy.

In an exemplary embodiment, the condition is a member selected from allergic rhinitis, asthma, adult respiratory distress syndrome, chronic pulmonary inflammation, chronic obstructive pulmonary disease, emphysema, bronchitis, mucus hypersecretion, silicosis, SARS infection and respiratory tract inflammation.

In an exemplary embodiment, the condition is a member selected from psoriasis, eczema, atopic dermatitis, contact dermatitis, or acne.

In an exemplary embodiment, the condition is a member selected from Guillain-Barre syndrome, Parkinson's disease, Huntington's disease, Alzheimer's disease, amyotrophic lateral sclerosis, multiple sclerosis and other demyelinating diseases, viral and bacterial meningitis, CNS trauma, spinal cord injury, seizures, convulsions, olivopontocerebellar atrophy, AIDS dementia complex, MERRF and MELAS syndromes, Leber's disease, Wemicke's encephalophathy, Rett syndrome, homocysteinuria, hyperprolinemia, hyperhomocysteinemia, nonketotic hyperglycinemia, hydroxybutyric aminoaciduria, sulfite oxidase deficiency, combined systems disease, lead encephalopathy, Tourett's syndrome, hepatic encephalopathy, drug addiction, drug tolerance, drug dependency, depression, anxiety and schizophrenia, aneurism, or epilepsy.

In an exemplary embodiment, the condition is a member selected from bone resorption diseases, osteopetrosis, osteoporosis, or osteoarthritis.

In an exemplary embodiment, the condition is a member selected from diabetes, systemic cachexia, cachexia secondary to infection or malignancy, cachexia secondary to acquired immune deficiency syndrome (AIDS), obesity, anorexia or bulimia nervosa. In an exemplary embodiment, the condition is a member selected from sepsis, HIV, HCV, malaria, infectious arthritis, leishmaniasis, Lyme disease, cancer, including but not limited to breast cancer, colon cancer, lung cancer, prostatic cancer, multiple myeloma, acute myelogenous leukemia, myelodysplastic syndrome, non-Hodgkins lymphoma, or follicular lymphoma, Castleman's disease, or drug resistance.

In an exemplary embodiment, the condition is a member selected from is bronchial asthma, rhinitis, influenza, stroke, myocardial infarction, thermal injury, adult respiratory distress syndrome (ARDS), multiple organ injury secondary to trauma, acute glomerulonephritis, dermatoses with acute inflammatory components, acute purulent meningitis, hemodialysis, leukopheresis, granulocyte transfusion associated syndromes, or necrotizing enterocolitis.

In an exemplary embodiment, the condition is a member selected from inflammatory bowel disease (IBD), psoriasis, rheumatoid arthritis (RA), multiple sclerosis (MS), neurodegenerative disorder, cardiovascular disease (CVD) and atherosclerosis, and metabolic disease (the metabolic syndrome and diabetes) as well as infection-related inflammation. In an exemplary embodiment, the condition is a neurodegenerative disorder which is a member selected from Alzheimer's disease and Parkinson disease. In an exemplary embodiment, the condition is inflammatory bowel disease which is selected from the group consisting of: Crohn's disease or ulcerative colitis. In an exemplary embodiment, the condition is a gastrointestinal complication. In an exemplary embodiment, the condition is diarrhea. In an exemplary embodiment, the condition is a member selected from celiac disease and non-specific colitis. In an exemplary embodiment, the condition is a liver disease. In an exemplary embodiment, the condition is a member selected from an autoimmune hepatitis, hepatitis C, primary biliary cirrhosis, primary sclerosing cholangitis, or fulminant liver failure. In an exemplary embodiment, the condition is a bone disease. In an exemplary embodiment, the condition is osteoporosis. In an exemplary embodiment, the condition is a pulmonary disorder. In an exemplary embodiment, the condition is a member selected from: allergic rhinitis, asthma, chronic obstructive pulmonary disease, chronic granulomatous inflammation, cystic fibrosis, and sarcoidosis. In an exemplary embodiment, condition is cardiovascular disease. In an exemplary embodiment, the cardiovascular disease is a member selected from atheroscleotic cardiac disease, congestive heart failure and restenosis. In an exemplary embodiment, the condition is a renal disease. In an exemplary embodiment, the condition is a member selected from glomerulonephritis and vasculitis. In an exemplary embodiment, the condition is a member selected from post-radiotherapy related disease or atherosclerosis. In yet another embodiment the condition is atopic dermatitis. In yet another embodiment the condition is actinic keratosis.

In an exemplary embodiment, the PDE4 inhibition is treating and/or preventing a disorder, and the disorder is a member selected from psoriasis, inflammatory arthritis, rheumatoid arthritis, asthma, chronic bronchitis, inflammatory bowel disease (IBD), chronic obstructive pulmonary disease (COPD), atopic dermatitis, urticaria, allergic rhinitis, allergic conjunctivitis, vernal conjunctivitis, colitis, esoniophilic granuloma, septic shock, reperfusion injury of the myocardium, reperfusion injury of the brain, chronic glomerulonephritis, endotoxic shock, adult respiratory distress syndrome, cystic fibrosis, arterial restenosis, atherosclerosis, keratosis, rheumatoid spondylitis, osteoarthritis, pyresis, diabetes mellitus, pneumoconiosis, chronic obstructive airways disease, toxic contact eczema, allergic contact eczema, atopic eczema, seborrheic eczema, lichen simplex, sunburn, pruritus in the anogenital area, alopecia areata, hypertrophic scars, discoid lupus erythematosus, systemic lupus erythematosus, follicular pyodermias, wide-area pyodermias, endogenous acne, exogenous acne, acne rosacea, Behcet's disease, anaphylactoid purpura nephritis, leukemia, multiple sclerosis, gastrointestinal disease and autoimmune disease. In an exemplary embodiment, the colitis is a member selected from ulcerative colitis, Crohn's colitis, diversion colitis, ischemic colitis, infectious colitis, fulminant colitis, chemical colitis, microscopic colitis, lymphocytic colitis, and atypical colitis. In an exemplary embodiment, the colitis is a member selected from ulcerative colitis and Crohn's colitis.

In an exemplary embodiment, the condition is psoriasis. In an exemplary embodiment, psoriasis is a member selected from plaque psoriasis, flexural psoriasis (inverse psoriasis), guttate psoriasis, pustular psoriasis, nail psoriasis, psoriatic arthritis and erythrodermic psoriasis. In an exemplary embodiment, the psoriasis is a member selected from plaque psoriasis and nail psoriasis.

In an exemplary embodiment, the disorder is a member selected from cognition impairment or decline and memory impairment. In an exemplary embodiment, the memory impairment is due to dementia. In an exemplary embodiment, the patient is suffering from memory impairment due to Alzheimer's disease, schizophrenia, Parkinson's disease, Huntington's disease, Pick's disease, Creutzfeld-Jakob disease, depression, aging, head trauma, stroke, CNS hypoxia, cerebral senility, multiinfarct dementia, an acute neuronal disease, age-related cognitive decline, HIV or a cardiovascular disease.

In an exemplary embodiment, the PDE4 inhibition is enhancing an effect, wherein the enhanced effect is cognition or memory.

In an exemplary embodiment, the invention provides a method for stimulating ovarian follicular growth in a female, comprising administering to a female a medicament comprising a compound described herein or a pharmaceutically acceptable salt thereof, whereby ovarian follicular growth is stimulated in the female. In an exemplary embodiment, the female is undergoing ovulation induction. In an exemplary embodiment, the female is undergoing controlled ovarian hyperstimulation. In an exemplary embodiment, the medicament is administered simultaneously, separately or sequentially with follicle stimulating hormone (FSH), or an agent having FSH activity, or an agent that stimulates endogenous FSH release.

The invention also provides a method of treating an inflammatory-related disease associated with cytokine expression levels, which comprises administering to an animal in need of such treatment the compound described herein or a pharmaceutically acceptable salt thereof. In an exemplary embodiment, the compound is according to a formula described herein.

In an exemplary embodiment, the invention provides a method of treating or preventing an inflammatory-related disease in an animal, the method comprising administering to the animal a therapeutically effective amount of a compound described herein or a pharmaceutically acceptable salt thereof, wherein the compound is in an amount sufficient to treat the inflammatory-related disease by inhibiting pro-inflammatory cytokine expression or by stimulating anti-inflammatory cytokine expression, but the amount is less than sufficient to substantially inhibit cyclin dependent kinases.

In an exemplary embodiment, the invention provides a method for inhibiting the production of an inflammatory cytokine by cells capable of producing the inflammatory cytokine, the method comprises contacting a cell with a therapeutic amount of the compound described herein or a pharmaceutically acceptable salt thereof, wherein production of the inflammatory cytokine by the cells is inhibited. In an exemplary embodiment, the therapeutic amount is sufficient to inhibit the production of the inflammatory cytokine protein between about 50% and about 99%.

In an exemplary embodiment, the invention provides a method for inhibiting an inflammatory response in an animal, the method comprising: contacting the animal with a therapeutic amount of the compound described herein or a pharmaceutically acceptable salt thereof, wherein the inflammatory response is inhibited.

In an exemplary embodiment, for any of the methods described herein, the animal is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, for any of the methods described herein, the animal is a member selected from a human, cattle, goat, pig, sheep, horse, cow, bull, dog, guinea pig, gerbil, rabbit, cat, chicken and turkey. In another exemplary embodiment, for any of the methods described herein, the animal is a human.

In an exemplary embodiment, for any of the methods described herein, a compound of the invention, a combination of the invention, a compound described herein or a pharmaceutically acceptable salt thereof, or combination described herein, and/or a pharmaceutical formulation described herein can be used.

### VII. Pharmaceutical Formulation

In another aspect, the invention provides a pharmaceutical formulation comprising: a) a compound of the invention; and b) a pharmaceutically acceptable excipient. In another aspect, the invention provides a pharmaceutical formulation comprising: a) a combination of the invention; and b) a pharmaceutically acceptable excipient. In an exemplary embodiment, the compound is according to a formula described herein. In an exemplary embodiment, the compound is according to an example described herein. In an exemplary embodiment, the compound of the invention or combination of the invention is a compound described herein or combination described herein, or a pharmaceutically acceptable salt thereof. In an exemplary embodiment, the compound of the invention is a compound described herein.

In an exemplary embodiment, the compound of the invention is present in the pharmaceutical formulation in an amount of between about 0.0001% to about 60% (w/w). In an exemplary embodiment, the amount is between about 0.01% to about 10% (w/w). In an exemplary embodiment, the amount is between about 0.1% to about 10% (w/w). In an exemplary embodiment, the amount is between about 0.25% to about 6% (w/w). In an exemplary embodiment, the amount is between about 0.5% to about 5% (w/w). In an exemplary embodiment, the amount is between about 0.1% and about 1.0% (w/w). In an exemplary embodiment, the amount is between about 1.0% and about 2.0% (w/w). In an exemplary embodiment, the amount is between about 2.0% and about 3.0% (w/w). In an exemplary embodiment, the amount is between about 3.0% and about 4.0% (w/w). In an exemplary embodiment, the amount is between about 4.0% and about 5.0% (w/w).

The pharmaceutical formulations of the invention can take a variety of forms adapted to the chosen route of administration. Those skilled in the art will recognize various synthetic methodologies that may be employed to prepare non-toxic pharmaceutical formulations incorporating the compounds described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The compositions of the invention may be administered orally, topically, parenterally, by inhalation or spray or rectally in dosage unit formulations containing conventional non-toxic pharmaceutically acceptable carriers, adjuvants and vehicles. It is further understood that the best method of administration may be a combination of methods. Oral administration in the form of a pill, capsule, elixir, syrup, lozenge, troche, or the like is particularly preferred. The term parenteral as used herein includes subcutaneous injections, intradermal, intravascular (e.g., intravenous), intramuscular, spinal, intrathecal injection or like injection or infusion techniques.

The pharmaceutical formulations containing compounds of the invention are preferably in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical formulations, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; lubricating agents, for example magnesium stearate, stearic acid or talc; and extenders and bulking agents, such as microcrystalline cellulose. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Other dispersing agents include hydrophilic polymers, electrolytes, Tween™ 60 or 80, PEG, polyvinylpyrrolidone (PVP; commercially known as Plasdone™), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone™, e.g., S-630), 4-(1,1,3,3-tetramethylbutyl)-phenol polymer with ethylene oxide and formaldehyde (also known as tyloxapol), poloxamers (e.g., Pluronics F68™, F88™, and F108™, which are block copolymers of ethylene oxide and propylene oxide); and poloxamines (e.g., Tetronic 9080, also known as Poloxamine 9080, which is a tetrafunctional block copolymer derived from sequential addition of propylene oxide and ethylene oxide to ethylenediamine (BASF Corporation, Parsippany, N.J.)). Additional excipients, for example sweetening, flavoring and coloring agents, may also be present.

Pharmaceutical formulations of the invention may also be in the form of oil-in-water emulsions and water-in-oil emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical formulations may be in the form of a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition of the invention may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Alternatively, the compositions can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For administration to non-human animals, the composition containing the therapeutic compound may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also added as a food or drink supplement for humans.

Dosage levels of the order of from about 5 mg to about 250 mg per kilogram of body weight per day and more preferably from about 25 mg to about 150 mg per kilogram of body weight per day, are useful in the treatment of the above-indicated conditions. The amount of active ingredient that may be combined with the carrier materials to produce a single dosage form will vary depending upon the condition being treated and the particular mode of administration. Dosage unit forms will generally contain between from about 1 mg to about 500 mg of an active ingredient.

Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

Preferred compounds of the invention will have desirable pharmacological properties that include, but are not limited to, oral bioavailability, low toxicity, low serum protein binding and desirable in vitro and in vivo half-lives. Penetration of the blood brain barrier for compounds used to treat CNS disorders is necessary, while low brain levels of compounds used to treat peripheral disorders are often preferred.

Assays may be used to predict these desirable pharmacological properties. Assays used to predict bioavailability include transport across human intestinal cell monolayers, including Caco-2 cell monolayers. Toxicity to cultured hepatocyctes may be used to predict compound toxicity. Penetration of the blood brain barrier of a compound in humans may be predicted from the brain levels of laboratory animals that receive the compound intravenously.

Serum protein binding may be predicted from albumin binding assays. Such assays are described in a review by Oravcova, et al. (Journal of Chromatography B (1996) volume 677, pages 1-27).

Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

The amount of the composition required for use in treatment will vary not only with the particular compound selected but also with the route of administration, the nature of the condition being treated and the age and condition of the patient and will ultimately be at the discretion of the attendant physician or clinician.

In an exemplary embodiment, the pharmaceutical composition described herein includes an additional active ingredient. In another exemplary embodiment, the additional active ingredient is a compound that has been approved for human use by the United States Food and Drug Administration. In another exemplary embodiment, the additional active ingredient is an immunosuppressive agent. In still another exemplary embodiment, the additional active ingredient is a member selected from corticosteroids, aminosalicylates, azathioprine (6-mercaptopurine), methotrexate and ciclosporine, etanercept, infliximab, adalimumab, alefacept, efalizumab and anakinra

In an exemplary embodiment, the additional active ingredient is a member selected from cilostazol, rolipram, roflumilast, piclamilast, CDP-840 and ariflo.

In still another exemplary embodiment, the additional active ingredient is a member selected from betamethasone, tacrolimus and pimecrolimus. In still another exemplary embodiment, the additional active ingredient is a member selected from an activated vitamin D analog and an arotinoid (an aromatic retinoic acid analog). In still another exemplary embodiment, the additional active ingredient is a member selected from carcipotriol, such as Tazorac (tazarotene).

In still another exemplary embodiment, the additional active ingredient is a member selected from penicillin G, amoxicillin, ampicillin, azlocillin, carbenicillin, cloxacillin, dicloxacillin, flucloxacillin, mezlocillin, nafcillin, pipericillin and ticarcillin.

In still another exemplary embodiment, the additional active ingredient is a member selected from tazobactam, sulbactam and clavulanic acid.

### VII. a) Topical Formulations

In a preferred embodiment, the methods of the invention can be employed through the topical application of the compounds described herein. Topical administration includes for example, transmucosal, transdermal, ungual and transungual routes of administration.

The compositions of the present invention comprises fluid or semi-solid vehicles that may include but are not limited to polymers, thickeners, buffers, neutralizers, chelating agents, preservatives, surfactants or emulsifiers, antioxidants, waxes or oils, emollients, sunscreens, and a solvent or mixed solvent system. The solvent or mixed solvent system is important to the formation because it is primarily responsible for dissolving the drug. The best solvent or mixed solvent systems are also capable of maintaining clinically relevant levels of the drug in solution despite the addition of a poor solvent to the formulation. The topical compositions useful in the subject invention can be made into a wide variety of product types. These include, but are not limited to, lotions, creams, gels, sticks, sprays, ointments, pastes, foams, mousses, masks, eye ointments, eye or ear drops, impregnated dressings, wipes, cleansers including soaps, body washes and shampoos, and make-up products, such as bases, blushes, lipsticks, and eye shadows, among others. These product types can comprise several types of carrier systems including, but not limited to particles, nanoparticles, and liposomes. If desired, disintegrating agents can be added, such as the cross-linked polyvinyl pyrrolidone, agar or alginic acid or a salt thereof such as sodium alginate. Techniques for formulation and administration can be found in Remington: The Science and Practice of Pharmacy, supra. The formulation can be selected to maximize delivery to a desired target site in the body. The formulations can also include various conventional colorants, fragrances, thickeners, preservatives, humectants, emollients, demulcents, solubilizing excipients, dispersants, penetration enhancers, plasticizing agents, preservatives, stabilizers, demulsifiers, wetting agents, sunscreens, emulsifiers, moisturizers, astringents, deodorants, and the like, which can be added to provide additional benefits such as, for example, improving the feel and/or appearance of the topical preparation.

Lotions, which are preparations that are to be applied to the skin, nail, hair, claw or hoof surface without friction, are typically liquid or semi-liquid preparations in which finely divided solid, waxy, or liquid are dispersed. Lotions will typically contain suspending agents to produce better dispersions as well as compounds useful for localizing and holding the active agent in contact with the skin, nail, hair, claw or hoof, e.g., methylcellulose, sodium carboxymethyl-cellulose, or the like.

Creams containing the active agent for delivery according to the present invention are viscous liquid or semisolid emulsions, either oil-in-water or water-in-oil. Cream bases are water-washable, and contain an oil phase, an emulsifier and an aqueous phase. The oil phase is generally comprised of petrolatum or a fatty alcohol, such as cetyl- or stearyl alcohol; the aqueous phase usually, although not necessarily, exceeds the oil phase in volume, and generally contains a humectant. The emulsifier in a cream formulation, as explained in Remington: The Science and Practice of Pharmacy, supra, is generally a nonionic, anionic, cationic or amphoteric surfactant.

A lotion or cream may include a relatively large aqueous phase and a relatively small oil phase. Furthermore, the lotions and creams of the invention may include the active compound “all-in-solution” in the oil phase so that substantially none of the active compound crystallizes out at room temperature. In one embodiment, the lotion or cream may comprise a biphasic system, that is, a system wherein a portion (from about 30 to about 75% by weight) of the active compound is in solution in the oil phase and the remainder is in suspension in the aqueous phase.

Gel formulations can also be used in connection with the present invention. As will be appreciated by those working in the field of topical drug formulation, gels are semisolid. Single-phase gels contain organic macromolecules distributed substantially uniformly throughout the carrier liquid, which is typically aqueous, but also may be a solvent or solvent blend. In various embodiments, conventional gelling agents can be used. In an exemplary embodiment, cellulose or its derivatives are used. In an exemplary embodiment, hydroxypropyl methyl cellulose, such as Methocel E4M, is used. Other gelling agents include methyl cellulose, hydroxypropyl cellulose, hydroxypropyl methyl cellulose, cellulose acetate, ethyl cellulose, methyl hydroxy ethyl cellulose, hydroxy ethyl cellulose, and cellulose gum. Cellulose based gelling agents, particularly hydroxymethylcellulose and hydroxypropyl methyl cellulose, are also useful in some embodiments. In some embodiments, cross-linked acrylic polymers including Carbopol may be used.

In one embodiment, the formulation of the invention is viscous enough to form a firm gel. In one embodiment, the viscosity is in the range of 25,000-300,000 cps (centipoise) or 75,000-200,000 cps, based on Brookfield (LV) analysis.

For ease of preparation, it may be convenient to prepare a first gel composition, named speed-gel herein, which can be used to add to other components in the formulation of a final composition for topical administration. There are several possible formulations of the speed-gel. For example, a speed-gel may be prepared by mixing lecithin organogel (L.O.), as a 1:1 (m/m) mixture of lecithin and isopropyl myristate, with LID oil (a 1:1 [m/m] mixture of L.O. and docusate sodium), dissolving additional docusate sodium powder into this mixture, and then adding aqueous urea.

Ointments, which are semisolid preparations, are typically based on petrolatum or other petroleum derivatives. As will be appreciated by the ordinarily skilled artisan, the specific ointment base to be used is one that provides for optimum delivery for the active agent chosen for a given formulation, and, preferably, provides for other desired characteristics as well, e.g., emolliency or the like. As with other carriers or vehicles, an ointment base should be inert, stable, nonirritating and non-sensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Examples of oleaginous ointment bases include White Ointment USP, Yellow Ointment NF, Oleic Acid USP, Olive Oil USP, Paraffin USP, Petrolatum NF, White Petrolatum USP, Spermaceti Wax USP, Synthetic Spermaceti NF, Starch Glycerite NF, White Wax USP, and Yellow Wax USP. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy, supra, for further information.

Useful formulations of the invention also encompass sprays and aerosols. Sprays generally provide the active agent in an aqueous and/or alcoholic solution which can be misted onto the skin, nail, hair, claw or hoof for delivery. Such sprays include those formulated to provide for concentration of the active agent solution at the site of administration following delivery, e.g., the spray solution can be primarily composed of alcohol or other like volatile liquid in which the drug or active agent can be dissolved. Upon delivery to the skin, nail, hair, claw or hoof, the carrier evaporates, leaving concentrated active agent at the site of administration. Examples of aerosol technology are disclosed in U.S. Pat. Nos. 6,682,716; 6,716,415; 6,716,417; 6,783,753; 7,029,658; and 7,033,575.

The topical pharmaceutical compositions may also comprise suitable solid or gel phase carriers. Examples of such carriers include but are not limited to calcium carbonate, calcium phosphate, various sugars, starches, cellulose derivatives, gelatin, and polymers such as polyethylene glycols.

The topical pharmaceutical compositions may also comprise a suitable emulsifier which refers to an agent that enhances or facilitates mixing and suspending oil-in-water or water-in-oil. The emulsifying agent used herein may consist of a single emulsifying agent or may be a nonionic, anionic, cationic or amphoteric surfactant or blend of two or more such surfactants; preferred for use herein are nonionic or anionic emulsifiers. Such surface-active agents are described in “McCutcheon's Detergent and Emulsifiers,” North American Edition, 1980 Annual published by the McCutcheon Division, MC Publishing Company, 175 Rock Road, Glen Rock, N.J. 07452, USA.

Examples of useful ionic surfactants include sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxyethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, sodium octyl sulfosuccinate, sodium undecylenamideo-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl,N,N-dimethylglycine), and ethoxylated amines (polyoxyethylene-15 coconut amine). For simplicity, typical counterions are provided above. It will be appreciated by one skilled in the art, however, that any bioacceptable counterion may be used. For example, although the fatty acids are shown as sodium salts, other cation counterions can also be used, such as, for example, alkali metal cations or ammonium. Formulations of the invention may include one or more of the ionic surfactants above.

Preferred for use herein are high molecular weight alcohols such as cetearyl alcohol, cetyl alcohol, stearyl alcohol, emulsifying wax, glyceryl monostearate, and oleyl alcohol. Other examples are ethylene glycol distearate, sorbitan tristearate, propylene glycol monostearate, sorbitan monooleate, sorbitan monostearate (SPAN 60), diethylene glycol monolaurate, sorbitan monopalmitate, sucrose dioleate, sucrose stearate (CRODESTA F-160), polyoxyethylene lauryl ether (BRIJ 30), polyoxyethylene (2) stearyl ether (BRIJ 72), polyoxyethylene (21) stearyl ether (BRIJ 721), polyoxyethylene monostearate (Myrj 45), polyoxyethylene (20) sorbitan monolaurate (TWEEN 20, polysorbate 20), polyoxyethylene (20) sorbitan monopalmitate (TWEEN 40, polysorbate 40), polyoxyethylene (20) sorbitan monostearate (TWEEN 60, polysorbate 60), polyoxyethylene (20) sorbitan monooleate (TWEEN 80, polysorbate 80), other non-ionic polyoxyalkylene derivatives of hexitol anhydride partial long chain fatty acid esters, and sodium oleate. In an exemplary embodiment, the emulsifier is octyldodecanol. In an exemplary embodiment, xanthan gum or a xanthan gum blend is used. Cholesterol and cholesterol derivatives may also be employed in externally used emulsions and promote w/o emulsions.

Especially suitable nonionic emulsifying agents are those with hydrophile-lipophile balances (HLB) of about 3 to 6 for w/o system and 8 to 18 for o/w system as determined by the method described by Paul L. Lindner in “Emulsions and Emulsion”, edited by Kenneth Lissant, published by Dekker, New York, N.Y., 1974, pages 188-190. More preferred for use herein are one or more nonionic surfactants that produce a system having HLB of about 8 to about 18.

Examples of such nonionic emulsifiers include but are not limited to “BRIJ 72”, the trade name for a polyoxyethylene (2) stearyl ether having an HLB of 4.9; “BRIJ 721”, the trade name for a polyoxyethylene (21) stearyl ether having an HLB of 15.5, “Brij 30”, the trade name for polyoxyethylene lauryl ether having an HLB of 9.7; “Polawax”, the trade name for emulsifying wax having an HLB of 8.0; “Span 60”, the trade name for sorbitan monostearate having an HLB of 4.7; “Crodesta F-160”, the trade name for sucrose stearate” having an HLB of 14.5. All of these materials are available from Ruger Chemicals Inc.; Croda; ICI Americas, Inc.; Spectrum Chemicals; and BASF. When the topical formulations of the present invention contain at least one emulsifying agent, each emulsifying agent is present in amount from about 0.5 to about 2.5 wt %, preferably 0.5 to 2.0%, more preferably 1.0% or 1.8%. Preferably the emulsifying agent comprises a mixture of steareth 21 (at about 1.8%) and steareth 2 (at about 1.0%).

The topical pharmaceutical compositions may also comprise suitable emollients. Emollients are materials used for the prevention or relief of dryness, as well as for the protection of the skin, nail, hair, claw or hoof. Useful emollients include, but are not limited to, hydrocarbon oils, waxes, silicone, cetyl alcohol, isopropyl myristate, stearyl alcohol, oleyl alcohol, octyl hydroxystearate, glycerin, other fatty alcohols including short or medium chain fatty alcohols having a carbon length of up to 18, medium or short chain fatty acid triglycerides, esters such as fatty acid esters, lecithins and related polar compounds such as phosphatidylcholine, phosphatidylethanolamine, phosphatidylserine, phosphatidylinositol, phosphatidic acid, lyso-phosphatidylcholine, lyso-phosphatidylethanolamine, and sphingomyelin and the like. Other suitable emollients include triglyceride oils like vegetable oils such as wheat germ, maize, sunflower, karite, castor, sweet almond, macadamia, apricot, soybean, cottonseed, alfalfa, poppy, pumpkinseed, sesame, cucumber, rapeseed, avocado, hazelnut, grape seed, blackcurrant seed, evening primrose, millet, barley, quinoa, olive, rye, safflower, candlenut, soya, palm, passion flower, or musk rose oil; triglycerides of caprylic/capric acid, such as those sold under the tradenames MIGLYOL™ (Condea Chemie, Germany) and CRODAMOL (Croda, Inc., Edison, N.J.); fatty alcohols such as caprylic alcohol, lauryl alcohol, myristyl alcohol, cetyl alcohol, and stearyl alcohol; and fatty esters such as oleyl acetate, isotridecyl benzoate, diisooctyl sebacate, isopropyl myristate, cetyl octanoate, isopropyl palmitate, butyl stearate, hexyl laurate, myristyl myristate, decyl oleate, hexyldecyl dimethyloctanoate, cetyl lactate, myristyl lactate, lanoline acetate, isocetyl stearate, isocetyl isostearate, cholesteryl 12-hydroxystearate, dipentaerythritol fatty acid ester, and isostearyl malate. A wide variety of suitable emollients are known and can be used herein. See e.g., Sagarin, Cosmetics, Science and Technology, 2nd Edition, Vol. 1, pp. 32-43 (1972), and U.S. Pat. No. 4,919,934, to Deckner et al., issued Apr. 24, 1990, both of which are incorporated herein by reference in their entirety. These materials are available from Ruger Chemical Co, (Irvington, N.J.).

When the topical formulations of the present invention contain at least one emollient, each emollient is present in an amount from about 0.1 to 15%, preferably 0.1 to about 3.0, more preferably 0.5, 1.0, or 2.5 wt %. Preferably the emollient is a mixture of cetyl alcohol, isopropyl myristate and stearyl alcohol in a 1/5/2 ratio. The emollient may also be a mixture of cetyl alcohol and stearyl alcohol in a 1/2 ratio.

The topical pharmaceutical compositions may also comprise suitable antioxidants, substances known to inhibit oxidation. Antioxidants suitable for use in accordance with the present invention include, but are not limited to, butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols such as vitamin E, and the like, including pharmaceutically acceptable salts and esters of these compounds. Preferably, the antioxidant is butylated hydroxytoluene, butylated hydroxyanisole, propyl gallate, ascorbic acid, pharmaceutically acceptable salts or esters thereof, or mixtures thereof. Most preferably, the antioxidant is butylated hydroxytoluene. These materials are available from Ruger Chemical Co, (Irvington, N.J.). Antioxidants that may be incorporated into the formulations of the invention include natural antioxidants prepared from plant extracts, such as extracts from aloe vera; avocado; chamomile; echinacea; ginko biloba; ginseng; green tea; heather; jojoba; lavender; lemon grass; licorice; mallow; oats; peppermint; St. John's wort; willow; wintergreen; wheat wild yam extract; marine extracts; and mixtures thereof.

When the topical formulations of the present invention contain at least one antioxidant, the total amount of antioxidant present is from about 0.001 to 0.5 wt %, preferably 0.05 to about 0.5 wt %, more preferably 0.1%.

The topical pharmaceutical compositions may also comprise suitable preservatives. Preservatives are compounds added to a pharmaceutical formulation to act as an anti-microbial agent. Among preservatives known in the art as being effective and acceptable in parenteral formulations are benzalkonium chloride, benzethonium, chlorohexidine, phenol, m-cresol, benzyl alcohol, methylparaben, propylparaben and other parabens, chlorobutanol, o-cresol, p-cresol, chlorocresol, phenylmercuric nitrate, thimerosal, benzoic acid, and various mixtures thereof. See, e.g., Wallhausser, K.-H., Develop. Biol. Standard, 24:9-28 (1974) (S. Krager, Basel). Preferably, the preservative is selected from methylparaben, propylparaben and mixtures thereof. These materials are available from Inolex Chemical Co (Philadelphia, Pa.) or Spectrum Chemicals.

When the topical formulations of the present invention contain at least one preservative, the total amount of preservative present is from about 0.01 to about 0.5 wt %, preferably from about 0.1 to 0.5%, more preferably from about 0.03 to about 0.15. Preferably the preservative is a mixture of methylparaben and propylparaben in a 5/1 ratio. When alcohol is used as a preservative, the amount is usually 15 to 20%.

The topical pharmaceutical compositions may also comprise suitable chelating agents to form complexes with metal cations that do not cross a lipid bilayer. Examples of suitable chelating agents include ethylene diamine tetraacetic acid (EDTA), ethylene glycol-bis(beta-aminoethyl ether)-N,N,N′,N′-tetraacetic acid (EGTA) and 8-Amino-2-[(2-amino-5-methylphenoxy)methyl]-6-methoxyquinoline-N,N,N′,N′-tetraacetic acid, tetrapotassium salt (QUIN-2). Preferably the chelating agents are EDTA and citric acid. A chelating agent may comprise salts of the above, such as edetate disodium, for example. These materials are available from Spectrum Chemicals.

When the topical formulations of the present invention contain at least one chelating agent, the total amount of chelating agent present is from about 0.005% to 2.0% by weight, preferably from about 0.05% to about 0.5 wt %, more preferably about 0.1% by weight.

The topical pharmaceutical compositions may also comprise suitable neutralizing agents used to adjust the pH of the formulation to within a pharmaceutically acceptable range. Examples of neutralizing agents include but are not limited to trolamine, tromethamine, sodium hydroxide, hydrochloric acid, sodium carbonate, citric acid, acetic acid and corresponding acids or bases thereof Such materials are available from are available from Spectrum Chemicals (Gardena, Calif.).

When the topical formulations of the present invention contain at least one neutralizing agent, the total amount of neutralizing agent present is from about 0.1 wt to about 10 wt %, preferably 0.1 wt % to about 5.0 wt %, and more preferably about 1.0 wt %. The neutralizing agent is generally added in whatever amount is required to bring the formulation to the desired pH. In one embodiment, the pH is about 6.0 to about 8.0. In one embodiment, the pH is about 3.0 to about 4.0.

The topical pharmaceutical compositions may also comprise suitable thickening or viscosity increasing agents. These components are diffusible compounds capable of increasing the viscosity of a polymer-containing solution through the interaction of the agent with the polymer. For example, CARBOPOL ULTREZ 10, polymethyl methacrylate (PMMA), and fumed silica may be used as a viscosity-increasing agent. These materials are available from Noveon Chemicals, Cleveland, Ohio. Other examples of thickeners include monoglycerides and fatty alcohols, fatty acid esters of alcohols having from about 3 to about 16 carbon atoms. Examples of suitable monoglycerides are glyceryl monostearate and glyceryl monopalmitate. Examples of fatty alcohols are cetyl alcohol and stearyl alcohol. Examples of suitable esters are myristyl stearate and cetyl stearate. The monoglyceride also functions as an auxiliary emulsifier. Other emollients or oleaginous material which may be employed include petrolatum, glyceryl monooleate, myristyl alcohol, and isopropyl palmitate. In one embodiment, the thickener is used in combination with an emulsifying agent.

When the topical formulations of the present invention contain at least one viscosity increasing agent, the total amount of viscosity increasing agent present is from about 0.25% to about 5.0% by weight, preferably from about 0.25% to about 1.0 wt %, and more preferably from about 0.4% to about 0.6% by weight.

The topical pharmaceutical compositions may also comprise a disintegrating agent including starch, e.g., a natural starch such as corn starch or potato starch, a pregelatinized starch such as National 1551 or Amijele™, or sodium starch glycolate such as Promogel™ or Explotab™; a cellulose such as a wood product, microcrystalline cellulose, e.g., Avicel™, Avicel™ PH101, Avicel™ PH102, Avicel™ PH105, Elcema™ P100, Emcocel™, Vivacel™, Ming Tia™, and Solka-Floc™, methylcellulose, croscarmellose, or a cross-linked cellulose, such as cross-linked sodium carboxymethylcellulose (Ac-Di-Sol™), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum™ HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

The topical pharmaceutical compositions may also comprise suitable nail penetration enhancers. Examples of nail penetration enhancers include mercaptan compounds, sulfites and bisulfites, keratolytic agents and surfactants. Nail penetration enhancers suitable for use in the invention are described in greater detail in Malhotra et al., J. Pharm. Sci., 91:2, 312-323 (2002), which is incorporated herein by reference in its entirety.

The topical pharmaceutical compositions may also comprise an anti-foaming anti-whitening agent that increases the elegancy of the cream or lotion and inhibits the formation of a white soapy look upon rubbing the cream or lotion on the skin. An example of such material includes silicone fluid. Other anti-foaming agents include simethicone, polyglycol, and sorbitan sesquioleate.

The topical pharmaceutical compositions may also comprise a post-foaming agent. “Post-foaming” refers to a gel that remains a gel as it is expelled from a container but foams up after it is spread over the skin. Post-foaming agents include saturated aliphatic hydrocarbons having from 4-6 carbon atoms, such as butane, pentane and hexane (in particular is opentane and isobutene). Other suitable post-foaming agents include partially, or wholly halogenated hydrocarbons, such as trichlorofluroethane. Also, mixtures of aliphatic and halogenated hydrocarbon propellants, or post-foaming agents can be used. Generally suitable post-foaming agents are those substances that have a low solubility in water, for example less than about 20 cc of gas in 100 grams of water at one atmosphere and 20° C.

The topical pharmaceutical compositions may also comprise one or more suitable solvents. The ability of any solid substance (solute) to dissolve in any liquid substance (solvent) is dependent upon the physical properties of the solute and the solvent. When solutes and solvents have similar physical properties the solubility of the solute in the solvent will be the greatest. This gives rise to the traditional understanding that “like dissolves like.” Solvents can be characterized in one extreme as non-polar, lipophilic oils, while in the other extreme as polar hydrophilic solvents. Oily solvents dissolve other non-polar substances by Van der Wals interactions while water and other hydrophilic solvents dissolve polar substances by ionic, dipole, or hydrogen bonding interactions. All solvents can be listed along a continuum from the least polar, i.e. hydrocarbons such as decane, to the most polar solvent being water. A solute will have its greatest solubility in solvents having equivalent polarity. Thus, for drugs having minimal solubility in water, less polar solvents will provide improved solubility with the solvent having polarity nearly equivalent to the solute providing maximum solubility. Most drugs have intermediate polarity, and thus experience maximum solubility in solvents such as propylene glycol or ethanol, which are significantly less polar than water. If the drug has greater solubility in propylene glycol (for example 8% (w/w)) than in water (for example 0.1% (w/w)), then addition of water to propylene glycol should decrease the maximum amount of drug solubility for the solvent mixture compared with pure propylene glycol. Addition of a poor solvent to an excellent solvent will decrease the maximum solubility for the blend compared with the maximum solubility in the excellent solvent.

When compounds are incorporated into topical formulations the concentration of active ingredient in the formulation may be limited by the solubility of the active ingredient in the chosen solvent and/or carrier. Non-lipophilic drugs typically display very low solubility in pharmaceutically acceptable solvents and/or carriers. For example, the solubility of some compounds in the invention in water is less than 0.00025% wt/wt. The solubility of the same compounds in the invention can be less than about 2% wt/wt in either propylene glycol or isopropyl myristate.

Examples of solubilizing excipients include polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, ionic surfactants, tocopherol esters, and sterol esters. In one embodiment of the present invention, ethylhexyl hydroxystearate is the solvent used to dissolve the compounds described herein. In one embodiment of the present invention, diethylene glycol monoethyl ether (DGME) is the solvent used to dissolve the compounds described herein. In one embodiment of the present invention, diethylene glycol monoethyl ether (DGME) is the solvent used to dissolve a compound of the invention. The compounds in the invention useful in the present formulation are believed to have a solubility of from about 10% wt/wt to about 25% wt/wt in DGME. In another embodiment a DGME water cosolvent system is used to dissolve the compounds described herein. In another embodiment a DGME water cosolvent system is used to dissolve a compound of the invention. The solvent capacity of DGME drops when water is added; however, the DGME/water cosolvent system can be designed to maintain the desired concentration of from about 0.1% to about 5% wt/wt active ingredient. Preferably the active ingredient is present from about 0.5% to about 3% wt/wt, and more preferably at about 1% wt/wt, in the as-applied topical formulations. Because DGME is less volatile than water, as the topical formulation evaporates upon application, the active agent becomes more soluble in the cream formulation. This increased solubility reduces the likelihood of reduced bioavailability caused by the drug precipitating on the surface of the skin, nail, hair, claw or hoof.

In one embodiment, the vehicle is lipophilic. Lipophilic materials include oleaginous material such as petrolatum, mineral oil thickened or gelled with polyethylene, high molecular weight paraffin waxes, mono and diglycerides of fatty acids gelled with high molecular weight fatty acids or polyamide complex of hydroxystearate, propylene glycol isostearate or isostearyl alcohol gelled with high molecular weight fatty acids, and mixtures thereof.

Liquid forms, such as lotions suitable for topical administration or suitable for cosmetic application, may include a suitable aqueous or nonaqueous vehicle with buffers, suspending and dispensing agents, thickeners, penetration enhancers, and the like. Solid forms such as creams or pastes or the like may include, for example, any of the following ingredients, water, oil, alcohol or grease as a substrate with surfactant, polymers such as polyethylene glycol, thickeners, solids and the like. Liquid or solid formulations may include enhanced delivery technologies such as liposomes, microsomes, microsponges and the like. Liposomal formulations, which help allow compounds to enter the skin, are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061.

Additionally, the compounds can be delivered using a sustained-release system, such as semipermeable matrices of solid hydrophobic polymers containing the therapeutic agent. Various sustained-release materials have been established and are well known by those skilled in the art. Thus, at least two different dosage forms, each of which contains a compound of the invention, may be formulated for topical administration by including such dosage forms in an oil-in-water emulsion, or a water-in-oil emulsion. In such a formulation, the delayed release dosage forms are in the continuous phase, and the delayed sustained release dosage form is in a discontinuous phase. The formulation may also be produced in a manner for delivery of three dosage forms as hereinabove described. For example, there may be provided an oil-in-water-in-oil emulsion, with oil being a continuous phase that contains the third delayed sustained release component, water dispersed in the oil containing a first delayed release dosage form, and oil dispersed in the water containing a second delayed release dosage form.

Topical treatment regimens according to the practice of this invention comprise applying the composition directly to the skin, nail, hair, claw or hoof at the application site, from one to several times daily.

Formulations of the present invention can be used to treat, ameliorate or prevent conditions or symptoms associated with bacterial infections, acne, inflammation and the like.

In an exemplary embodiment, the pharmaceutical formulation includes a simple solution. In an exemplary embodiment, the simple solution includes a polyether. In an exemplary embodiment, the polyether is polyethylene glycol or polypropylene glycol. In an exemplary embodiment, the simple solution includes an alcohol. In an exemplary embodiment, the alcohol is methanol, ethanol, propanol, isopropanol or butanol. In an exemplary embodiment, the simple solution includes a polyether and an alcohol. In another exemplary embodiment, the simple solution includes a polypropylene glycol and ethanol. In another exemplary embodiment, the simple solution is a member selected from about 10% polypropylene glycol and about 90% ethanol; about 20% polypropylene glycol and about 80% ethanol; about 30% polypropylene glycol and about 70% ethanol; about 40% polypropylene glycol and about 60% ethanol; about 50% polypropylene glycol and about 50% ethanol; about 60% polypropylene glycol and about 40% ethanol; about 70% polypropylene glycol and about 30% ethanol; about 80% polypropylene glycol and about 20% ethanol; about 90% polypropylene glycol and about 10% ethanol.

In an exemplary embodiment, the simple solution includes acetone. In an exemplary embodiment, the simple solution includes acetone and an alcohol. In an exemplary embodiment, the simple solution includes acetone and a member selected from methanol, ethanol, propanol, isopropanol or butanol. In an exemplary embodiment, the simple solution includes acetone, an alcohol and a polyether. In another exemplary embodiment, the simple solution includes acetone, an alcohol and a member selected from polyethylene glycol and polypropylene glycol. In an exemplary embodiment, the simple solution includes acetone and ethanol. In another exemplary embodiment, the simple solution is a member selected from about 10% acetone and about 90% ethanol; about 20% acetone and about 80% ethanol; about 30% acetone and about 70% ethanol; about 40% acetone and about 60% ethanol; about 50% acetone and about 50% ethanol; about 60% acetone and about 40% ethanol; about 70% acetone and about 30% ethanol; about 80% acetone and about 20% ethanol; about 90% acetone and about 10% ethanol.

In an exemplary embodiment, the pharmaceutical formulation is a lacquer.

### VII. b) Additional Active Agents

The following are examples of the cosmetic and pharmaceutical agents that can be added to the topical pharmaceutical formulations of the present invention. The following agents are known compounds and are readily available commercially.

Anti-inflammatory agents include, but are not limited to, bisabolol, mentholatum, dapsone, aloe, hydrocortisone, and the like.

Vitamins include, but are not limited to, Vitamin B, Vitamin E, Vitamin A, Vitamin D, and the like and vitamin derivatives such as tazarotene, calcipotriene, tretinoin, adapalene and the like.

Anti-aging agents include, but are not limited to, niacinamide, retinol and retinoid derivatives, AHA, Ascorbic acid, lipoic acid, coenzyme Q 10, beta hydroxy acids, salicylic acid, copper binding peptides, dimethylaminoethyl (DAEA), and the like.

Sunscreens and or sunburn relief agents include, but are not limited to, PABA, jojoba, aloe, padimate-O, methoxycinnamates, proxamine HCl, lidocaine and the like. Sunless tanning agents include, but are not limited to, dihydroxyacetone (DHA). Ultraviolet (UV) light blockers include, for example, amino benzoic acids, benzophenones, camphors, cinnamates, dibenzoyl methanes, salicylates, metal oxides, and mixtures thereof.

Psoriasis-treating agents and/or acne-treating agents include, but are not limited to, salicylic acid, benzoyl peroxide, coal tar, selenium sulfide, zinc oxide, pyrithione (zinc and/or sodium), tazarotene, calcipotriene, tretinoin, adapalene and the like.

Agents that are effective to control or modify keratinization, including without limitation: tretinoin, tazarotene, and adapalene.

The compositions comprising an compound/active agent described herein, and optionally at least one of these additional agents, are to be administered topically. In a primary application, this leads to the compounds of the invention and any other active agent working upon and treating the skin, nail, hair, claw or hoof. Alternatively, any one of the topically applied active agents may also be delivered systemically by transdermal routes.

In such compositions an additional cosmetically or pharmaceutically effective agent, such as an anti-inflammatory agent, vitamin, anti-aging agent, sunscreen, and/or acne-treating agent, for example, is usually a minor component (from about 0.001% to about 20% by weight or preferably from about 0.01% to about 10% by weight) with the remainder being various vehicles or carriers and processing aids helpful for forming the desired dosing form.

### VII. c) Testing

Preferred compounds for use in the present topical formulations will have certain pharmacological properties. Such properties include, but are not limited to, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcova et al. (1996, J. Chromat. B677: 1-27). Compound half-life is inversely proportional to the frequency of dosage of a compound. In vitro half-lives of compounds may be predicted from assays of microsomal half-life as described by Kuhnz and Gleschen (Drug Metabolism and Disposition, (1998) volume 26, pages 1120-1127).

Toxicity and therapeutic efficacy of such compounds can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

### VII. d) Administration

For any compound used in the method of the invention, the therapeutically effective dose can be estimated initially from cell culture assays, as disclosed herein. For example, a dose can be formulated in animal models to achieve a circulating concentration range that includes the EC₅₀ (effective dose for 50% increase) as determined in cell culture, i.e., the concentration of the test compound which achieves a half-maximal inhibition of bacterial cell growth. Such information can be used to more accurately determine useful doses in humans.

In general, the compounds prepared by the methods, and from the intermediates, described herein will be administered in a therapeutically or cosmetically effective amount by any of the accepted modes of administration for agents that serve similar utilities. It will be understood, however, that the specific dose level for any particular patient will depend upon a variety of factors including the activity of the specific compound employed, the age, body weight, general health, sex, diet, time of administration, route of administration, and rate of excretion, drug combination, the severity of the particular disease undergoing therapy and the judgment of the prescribing physician. The drug can be administered from once or twice a day, or up to 3 or 4 times a day.

Dosage amount and interval can be adjusted individually to provide plasma levels of the active moiety that are sufficient to maintain bacterial cell growth inhibitory effects. Usual patient dosages for systemic administration range from 0.1 to 1000 mg/day, preferably, 1-500 mg/day, more preferably 10-200 mg/day, even more preferably 100-200 mg/day. Stated in terms of patient body surface areas, usual dosages range from 50-91 mg/m²/day.

The amount of the compound in a formulation can vary within the full range employed by those skilled in the art. Typically, the formulation will contain, on a weight percent (wt %) basis, from about 0.01-10 wt % of the drug based on the total formulation, with the balance being one or more suitable pharmaceutical excipients. Preferably, the compound is present at a level of about 0.1-3.0 wt %, more preferably, about 1.0 wt %.

In an exemplary embodiment, the pharmaceutical formulation is an ointment, and comprises a compound described herein or combination described herein or a pharmaceutically acceptable salt thereof.

In another exemplary embodiment, the pharmaceutical formulation includes a compound described herein or combination described herein or a pharmaceutically acceptable salt thereof and at least one surfactant described herein. In another exemplary embodiment, the formulation comprises a hydroxystearate. In another exemplary embodiment, the hydroxystearate is a member selected from glyceryl monostearate, ethylhexyl hydroxystearate and octyl hydroxystearate.

In another exemplary embodiment, the pharmaceutical formulation includes a compound described herein or a combination described herein or a pharmaceutically acceptable salt thereof and an alcohol. In another exemplary embodiment, the alcohol is a long chain alcohol or a fatty alcohol. In another exemplary embodiment, the alcohol is a member selected from benzyl alcohol, octyldodecanol, stearyl alcohol, cetyl alcohol, oleyl alcohol. In an exemplary embodiment, the formulation comprises a member selected from benzyl alcohol, octyl comprises at least one compound which is a member selected from hydrocarbon oils, waxes, silicone, cetyl alcohol, isopropyl myristate, stearyl alcohol, oleyl alcohol, ethylhexyl hydroxystearate, octyl hydroxystearate, glycerin, other fatty alcohols hydroxystearate.

In another exemplary embodiment, the pharmaceutical formulation comprises a compound of the invention and at least one emollient described herein.

In another exemplary embodiment, the pharmaceutical formulation includes a compound of the invention, and petrolatum.

In an exemplary embodiment, the pharmaceutical formulation comprises a compound described herein or combination described herein or a pharmaceutically acceptable salt thereof and petrolatum. In an exemplary embodiment, the pharmaceutical formulation comprises a compound described herein or combination described herein or a pharmaceutically acceptable salt thereof and a member selected from hydrocarbon oils, waxes, silicone, cetyl alcohol, isopropyl myristate, stearyl alcohol, oleyl alcohol, ethylhexyl hydroxystearate, octyl hydroxystearate, glycerin, other fatty alcohols hydroxystearate. In an exemplary embodiment, the pharmaceutical formulation comprises a compound described herein or combination described herein or a pharmaceutically acceptable salt thereof and ethylhexyl hydroxystearate and/or octyl hydroxystearate. In an exemplary embodiment, the pharmaceutical formulation comprises a compound described herein or combination described herein or a pharmaceutically acceptable salt thereof, petrolatum and a member selected from hydrocarbon oils, waxes, silicone, cetyl alcohol, isopropyl myristate, stearyl alcohol, oleyl alcohol, ethylhexyl hydroxystearate, octyl hydroxystearate, glycerin, other fatty alcohols hydroxystearate. In an exemplary embodiment, the pharmaceutical formulation comprises a compound described herein or described herein or a pharmaceutically acceptable salt thereof, petrolatum, oleyl alcohol and ethylhexyl hydroxystearate.

In an exemplary embodiment, the pharmaceutical formulation is a cream, and comprises a compound described herein or combination described herein or a pharmaceutically acceptable salt thereof.

In another exemplary embodiment, the pharmaceutical formulation comprises a compound described herein or combination described herein or a pharmaceutically acceptable salt thereof and a preservative. In an exemplary embodiment, the preservative is a member selected from benzalkonium chloride, benzethonium, chlorohexidine, phenol, m-cresol, benzyl alcohol, methylparaben, propylparaben and other parabens, chlorobutanol, o-cresol, p-cresol, chlorocresol, phenylmercuric nitrate, thimerosal, benzoic acid, and various mixtures thereof. In an exemplary embodiment, the preservative is a paraben. In an exemplary embodiment, the preservative is a member selected from methyl paraben and propyl paraben.

In another exemplary embodiment, the pharmaceutical formulation comprises a compound described herein or combination described herein or a pharmaceutically acceptable salt thereof and a chelating agent. In an exemplary embodiment, the chelating agent is edetate sodium.

Exemplary embodiments are summarized herein below.

In an exemplary embodiment, the invention provides a compound having a structure according to the formula:

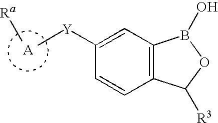

wherein A is a member selected from cycloalkyl, heterocycloalkyl, aryl and heteroaryl; Y is a member selected from O and —S(O)₂NH— wherein the sulfur in —S(O)₂NH— is covalently attached to A; R³ is a member selected from H, cyano and substituted alkyl; R^{a} is a member selected from H, —OR²⁰, —NR²⁰R²¹, —SR²⁰, —S(O)R²⁰, —S(O)₂R²⁰, —S(O)₂NR²⁰R²¹, —C(O)R²⁰, —C(O)OR²⁰, —C(O)NR²⁰R²¹, nitro, cyano, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl wherein each R²⁰ and each R²¹ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl, with the proviso that R²⁰ and R²¹, together with the nitrogen to which they are attached, are optionally combined to form a 5- to 7-membered substituted or unsubstituted heterocycloalkyl ring; with the proviso that when Y is O, R³ is a member selected from cyano and substituted alkyl; with the proviso that when Y is —S(O)₂NH—, R³ is H, and R^{a} is not H or unsubstituted alkyl or halosubstituted alkyl, and salts thereof.

In an exemplary embodiment, according to the above paragraph, the compound has a structure according to the following formula:

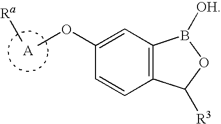

In an exemplary embodiment, according to any of the above paragraphs, the compound has a structure which is a member selected from:

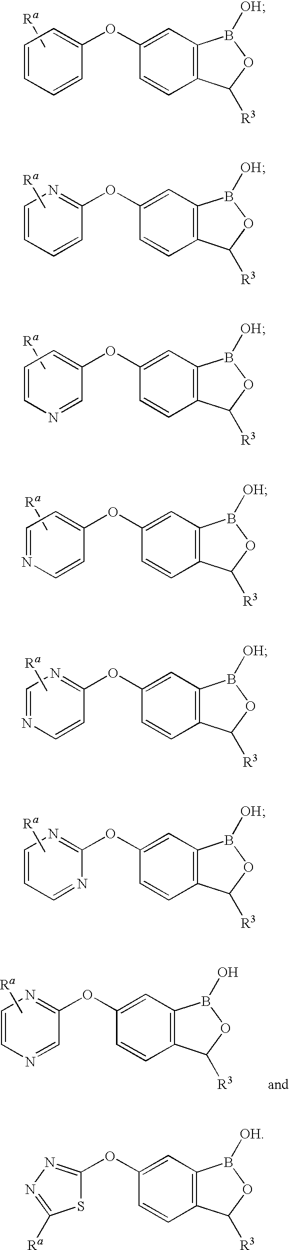

In an exemplary embodiment, according to any of the above paragraphs, R^{a} is a member selected from H, F, Cl, —OR^{20a} and —C(O)OR^{20b}, wherein R^{20a} is alkyl, optionally substituted with a member selected from NH₂ and phenyl, wherein R^{20b} is unsubstituted alkyl.

In an exemplary embodiment, according to any of the above paragraphs, R^{a} is —O(CH₂)ₙNH₂, wherein n is an integer selected from 1 to 6.

In an exemplary embodiment, according to any of the above paragraphs, n is 2 or 3 or 4.

In an exemplary embodiment, according to any of the above paragraphs, the compound has a structure according to the formula:

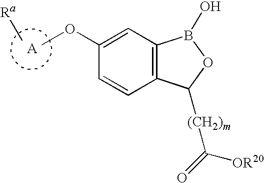

wherein m is an integer selected from 1 to 6 and R²⁰ is a member selected from H and unsubstituted alkyl.

In an exemplary embodiment, according to any of the above paragraphs, m is 1 or 2 or 3.

In an exemplary embodiment, according to any of the above paragraphs, the compound has a structure according to the formula:

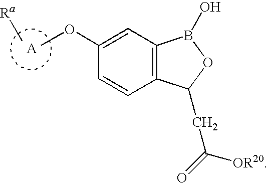

In an exemplary embodiment, according to any of the above paragraphs, R²⁰ is H.

In an exemplary embodiment, according to any of the above paragraphs, R²⁰ is C₁ or C₂ or C₃ unsubstituted alkyl.

In an exemplary embodiment, according to any of the above paragraphs, R³ is —CH₂COOH or —CH₂COOCH₃ or —CH₂COOCH₂CH₃.

In an exemplary embodiment, according to any of the above paragraphs, the compound has a structure according to the formula:

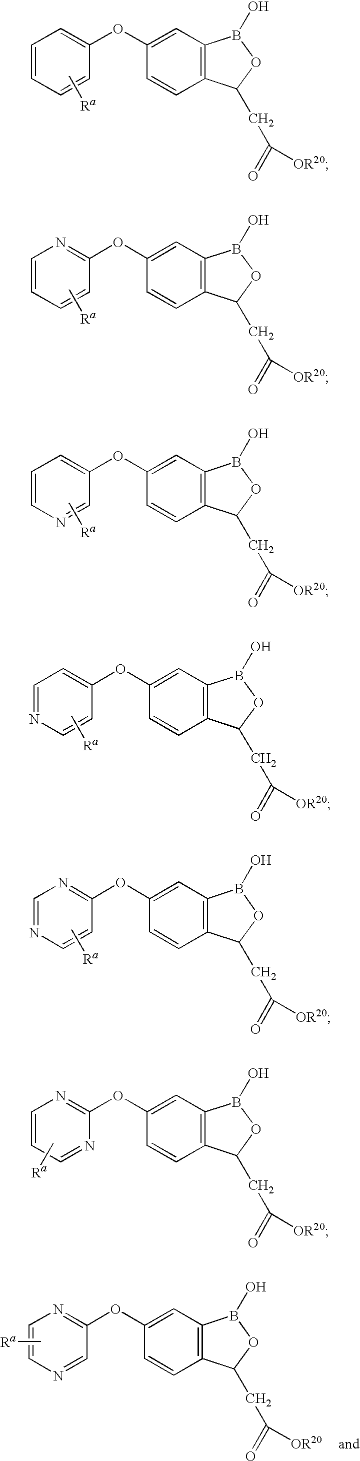

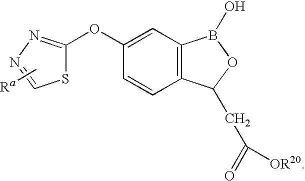

In an exemplary embodiment, according to any of the above paragraphs, the compound has a structure according to the formula:

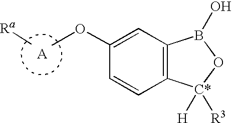

wherein C* is a carbon atom which is a stereocenter which has a configuration of (R) or (S).

In an exemplary embodiment, according to any of the above paragraphs, C* is a stereocenter which has a (R) configuration.

In an exemplary embodiment, according to any of the above paragraphs, the compound has a structure according to the formula:

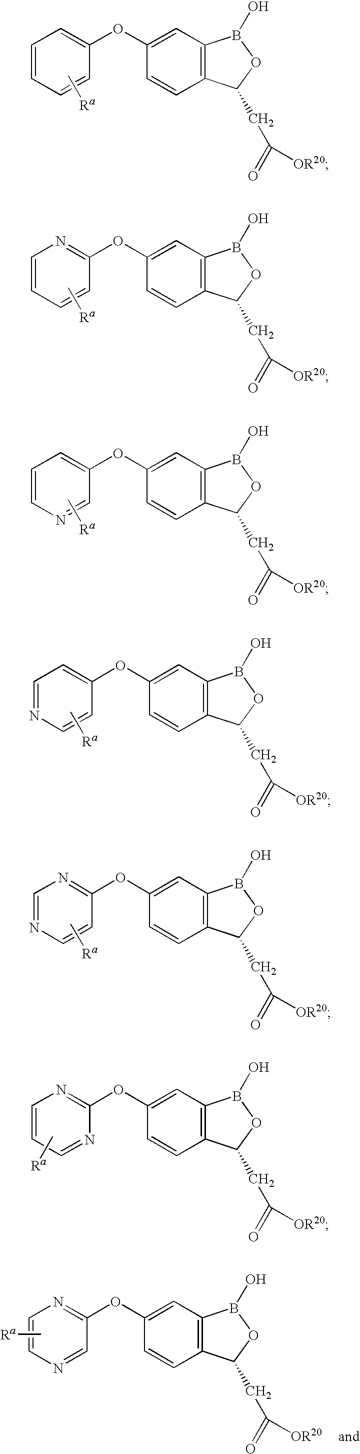

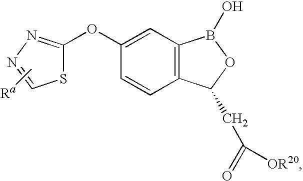

wherein R²⁰ is a member selected from H and unsubstituted alkyl.

In an exemplary embodiment, according to any of the above paragraphs, R²⁰ is H.

In an exemplary embodiment, according to any of the above paragraphs, the compound has a structure according to the formula:

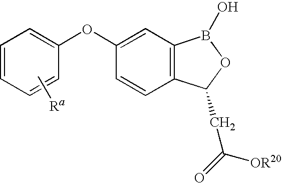

wherein R^{a} is —O(CH₂)ₙNH₂, wherein n is an integer selected from 1 to 6.

In an exemplary embodiment, according to any of the above paragraphs, the compound has a structure which is:

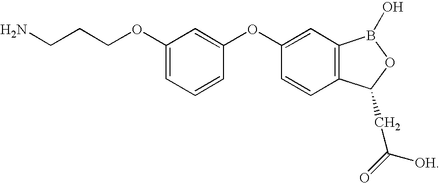

In an exemplary embodiment, according to any of the above paragraphs, the compound has a structure according to the formula:

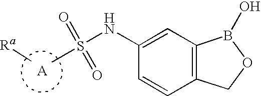

In an exemplary embodiment, according to any of the above paragraphs, A is a member selected from phenyl, pyridinyl, furanyl, thiophenyl, pyrazolyl, imidazolyl, thiazolyl, triazolyl, and piperidinyl.

In an exemplary embodiment, according to any of the above paragraphs, R^{a} is a member selected from cyano, nitro, aminoalkyl, hydroxyalkyl, —C(O)(CH₂)ₘ₁CH₃, —COOH, —C(O)O(CH₂)ₘ₁CH₃, —O(CH₂)ₘ₁CH₃, —O(CH₂)ₘ₁CF₃, —O(CH₂)ₘ₁CHF₂, —OH, —NH₂, —NHCH₃, —NHC(O)H, —NHC(O)(CH₂)ₘ₁CH₃, —NHOH, —NHS(O)₂NH₂, —NH₂S(O)₂CH₃, —S(O)₂CH₃, wherein m1 is an integer which is a member selected from 0 to 3.

In an exemplary embodiment, according to any of the above paragraphs, the compound has a structure according to the formula:

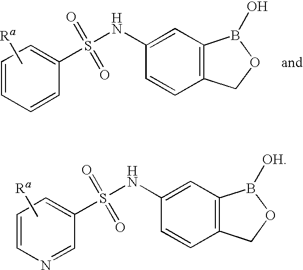

In an exemplary embodiment, according to any of the above paragraphs, the compound has a structure according to the formula:

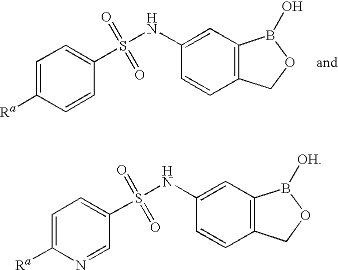

In an exemplary embodiment, according to any of the above paragraphs, R^{a} is a member selected from OH and NH₂.

In an exemplary embodiment, the invention is a combination comprising: a) a compound according to any of the above paragraphs, or a pharmaceutically acceptable salt thereof; and b) a therapeutically active agent.

In an exemplary embodiment, according to any of the above paragraphs, the therapeutically active agent is an antibiotic which comprises a β-lactam moiety.

In an exemplary embodiment, the invention is a pharmaceutical formulation comprising: a) a compound or a combination according to any of the above paragraphs, or a pharmaceutically acceptable salt thereof; and b) a pharmaceutically acceptable excipient.

In an exemplary embodiment, according to any of the above paragraphs, the pharmaceutical formulation is a unit dosage form.

In an exemplary embodiment, according to any of the above paragraphs, the pharmaceutical formulation is a member selected from an oral unit dosage form and a topical unit dosage form.

In an exemplary embodiment, the invention is a method of treating a bacterial infection comprising: administering to an animal suffering from said infection an effective amount of a compound according to any of the above paragraphs, or a pharmaceutically-acceptable salt thereof, and an effective amount of an antibiotic, or a pharmaceutically acceptable salt thereof, wherein said antibiotic comprises a β-lactam moiety, thereby treating the bacterial infection.

In an exemplary embodiment, according to any of the above paragraphs, a bacteria involved with the infection is resistant to said antibiotic.

In an exemplary embodiment, according to any of the above paragraphs, the antibiotic is a member selected from a penicillin, cephalosporin, monobactam, carbapenem and derivatives thereof.

In an exemplary embodiment, according to any of the above paragraphs, the antibiotic is a penicillin or derivatives thereof.

In an exemplary embodiment, according to any of the above paragraphs, the penicillin is a member selected from narrow spectrum penicillins, narrow spectrum penicillinase-resistant penicillins, narrow spectrum β-lactamase-resistant penicillins, moderate spectrum penicillins, broad spectrum penicillins and extended spectrum penicillins

In an exemplary embodiment, according to any of the above paragraphs, the penicillin is a narrow spectrum penicillin which is a member selected from benzathine penicillin, benzylpenicillin (penicillin G), phenoxymethylpenicillin (penicillin V) and procaine penicillin.

In an exemplary embodiment, according to any of the above paragraphs, the penicillin is a narrow spectrum penicillinase-resistant penicillins which is a member selected from methicillin, dicloxacillin and flucloxacillin.

In an exemplary embodiment, according to any of the above paragraphs, the penicillin is a narrow spectrum β-lactamase-resistant penicillin which is temocillin.

In an exemplary embodiment, according to any of the above paragraphs, the penicillin is a moderate spectrum penicillin which is a member selected from amoxicillin and ampicillin.

In an exemplary embodiment, according to any of the above paragraphs, the penicillin is a broad spectrum penicillin which is a member selected from co-amoxiclav (amoxicillin and clavulanic acid).

In an exemplary embodiment, according to any of the above paragraphs, the penicillin is an extended spectrum penicillin, which is a member selected from azlocillin, carbenicillin, ticarcillin, mezlocillin and piperacillin.

In an exemplary embodiment, according to any of the above paragraphs, the antibiotic is a cephalosporin or a derivative thereof.

In an exemplary embodiment, according to any of the above paragraphs, the cephalosporin is a member selected from a first-generation cephalosporin, second-generation cephalosporin, second-generation cephamycin, third-generation cephalosporin and fourth-generation cephalosporin.

In an exemplary embodiment, according to any of the above paragraphs, the cephalosporin is a member selected from cefalexin, cephalothin and cefazolin.

In an exemplary embodiment, according to any of the above paragraphs, the cephalosporin is a member selected from cefaclor, cefuroxime and cefamandole.

In an exemplary embodiment, according to any of the above paragraphs, the cephalosporin is a member selected from cefotetan and cefoxitin.

In an exemplary embodiment, according to any of the above paragraphs, the cephalosporin is a member selected from ceftriaxone, cefotaxime, cefpodoxime and ceftazidime.

In an exemplary embodiment, according to any of the above paragraphs, the cephalosporin is a member selected from cefepime and cefpirome.

In an exemplary embodiment, according to any of the above paragraphs, the antibiotic is a monobactam.

In an exemplary embodiment, according to any of the above paragraphs, the monobactam is aztreonam.

In an exemplary embodiment, according to any of the above paragraphs, the antibiotic is a carbapenem.

In an exemplary embodiment, according to any of the above paragraphs, the carbapenem is a member selected from imipenem, cilastatin, meropenem, ertapenem and faropenem.

In an exemplary embodiment, according to any of the above paragraphs, said animal is a human.

In an exemplary embodiment, the invention is a method of killing or inhibiting the growth of a bacteria, said method comprising: contacting said bacteria with an effective amount of a compound or a combination according to any of the above paragraphs, or a pharmaceutically acceptable salt thereof, thereby killing or inhibiting the growth of the bacteria.

In an exemplary embodiment, according to any of the above paragraphs, the method further comprises contacting said bacteria with an effective amount of an antibiotic, or a pharmaceutically acceptable salt thereof, wherein said antibiotic comprises a β-lactam moiety.

In an exemplary embodiment, according to any of the above paragraphs, the bacteria is resistant to said antibiotic.

In an exemplary embodiment, the invention is a method of inhibiting a β-lactamase, comprising contacting the β-lactamase with an effective amount of a compound according to any of the above paragraphs, or a pharmaceutically acceptable salt thereof, thereby inhibiting the β-lactamase.

In an exemplary embodiment, according to any of the above paragraphs, the β-lactamase is a member selected from a Group 1 β-lactamase, a Group 2 β-lactamase, a Group 3 β-lactamase, and a Group 4 β-lactamase.

In an exemplary embodiment, according to any of the above paragraphs, the Group 1 β-lactamase is a cephalosporinase.

In an exemplary embodiment, according to any of the above paragraphs, the Group 2 β-lactamase is a member selected from penicillinase, a Group 2b, Group 2be, Group 2br, carbenicillinase, cloxacilanase, cephalosporinase and carbapenamase.

In an exemplary embodiment, according to any of the above paragraphs, the Group 3 β-lactamase is a metallo-β-lactamase.

In an exemplary embodiment, according to any of the above paragraphs, the Group 4 β-lactamase is a penicillinase.

In an exemplary embodiment, according to any of the above paragraphs, the β-lactamase is a member selected from a class A β-lactamase, a class B β-lactamase, a class C β-lactamase, and a class D β-lactamase.

In an exemplary embodiment, according to any of the above paragraphs, the class A β-lactamase is a member selected from a TEM β-lactamase, SHV β-lactamase, CTX-M β-lactamase and a KPC β-lactamase.

In an exemplary embodiment, according to any of the above paragraphs, the class C β-lactamase is a member selected from a CMY β-lactamase and a AmpC β-lactamase.

In an exemplary embodiment, according to any of the above paragraphs, the class D β-lactamase is an OXA β-lactamase.

In an exemplary embodiment, according to any of the above paragraphs, the β-lactamase is a metallo β-lactamase.

In an exemplary embodiment, according to any of the above paragraphs, the metallo β-lactamase is a member selected from an IMP carbapenemase and a VIM β-lactamase.

In an exemplary embodiment, the invention is a method of treating a bacterial infection comprising: administering to an animal suffering from said infection an effective amount of a compound according to any of the above paragraphs, or a pharmaceutically-acceptable salt thereof, thereby treating the bacterial infection.

In an exemplary embodiment, the invention is a method of inhibiting the editing domain of a t-RNA synthetase, comprising: contacting the synthetase with an effective amount of a compound according to any of the above paragraphs, or a pharmaceutically-acceptable salt thereof, thereby inhibiting the synthetase.

In an exemplary embodiment, according to any of the above paragraphs, the synthetase is a leucyl t-RNA synthetase.

In an exemplary embodiment, the invention is the use of a compound or a combination according to any of the above paragraphs, or a pharmaceutically acceptable salt thereof, in the manufacture of a medicament for the treatment and/or prophylaxis of bacterial infection.

The invention is further illustrated by the Examples that follow. The Examples are not intended to define or limit the scope of the invention.

### EXAMPLES

Proton NMR are recorded on Varian AS 300 spectrometer and chemical shifts are reported as δ (ppm) down field from tetramethylsilane. Mass spectra are determined on Micromass Quattro II.

### ABBREVIATIONS

AcOH acetic acid
ACTBr cetyltrimethylammonium bromide
Cs₂CO₃ cesium carbonate
DCM dichloromethane
DIEA diisopropylethylamine
DMAP 4-(dimethylamino)pyridine
DME 1,2-dimethoxyethane
DMF N,N-dimethylformamide
DMSO dimethylsulfoxide
EtOAc ethyl acetate
EtOH ethanol
Et₂O diethyl ether
h hour(s)
HATU O-(7-azabenzotriazol-1-yl)-N,N,N′,N′-tetramethyluronium hexafluorophosphate
HCl hydrochloric acid
HPLC high pressure liquid chromatography
ISCO Companion automated flash chromatography equipment with
fraction analysis by UV absorption available from Presearch.
K₂OAc potassium acetate
K₂CO₃ potassium carbonate
LiAlH₄ or LAH lithium aluminum hydride
LDA lithium diisopropylamide
LHMDS lithium bis(trimethylsilyl) amide
KHMDS potassium bis(trimethylsilyl) amide
LiOH lithium hydroxide
MeCN acetonitrile
MeOH methanol
MgSO₄ magnesium sulfate
mins or min minutes
Mp or MP melting point
NaOH sodium hydroxide
Na₂SO₄ sodium sulfate
NH₄Cl ammonium chloride
N₂ nitrogen
NMM N-methyl morpholine
n-BuLi n-butyllithium
PdCl₂(pddf) [1,1′-bis(diphenylphosphino)ferrocene]palladium(II) chloride 1:1 complex with dichloro-methane
RT or rt room temperature
TFA trifluoroacetic acid
Tf₂O trifluoromethanesulfonic anhydride
THF tetrahydrofuran
H₂O water

### Example 1

### E1. 3H-Benzo[c][1,2]oxaborole-1,6-diol

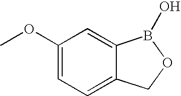

### Step 1 Trifluoro-methanesulfonic acid 2-formyl-5-methoxy-phenyl ester

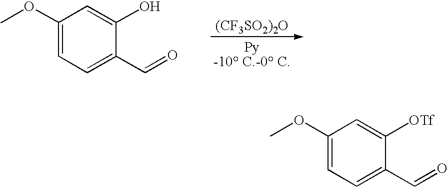

To a solution of 2-hydroxy-4-methoxy-benzaldehyde (30.0 g, 0.197 mol) and pyridine (77.98 g, 0.986 mol) in dichloromethane (120 mL) was slowly added Tf₂O (83.44 g, 0.296 mol) at −10 to 0° C. over a 2.5 h period. The mixture was stirred at 0° C. for 30 min. Ice-water (150 mL) was added, and the mixture was acidified with diluted hydrochloric acid to pH 2. The resulting mixture was extract with 50% EtOAc/hexanes (2×400 mL). The extract was washed with brine, dried and concentrated to dryness to give 51.01 g (91.1% yield) of product as pale-yellow oil.

¹H NMR (400 MHz, CDCl₃) δ 10.13 (s, 1H), 7.95 (d, J=8.79 Hz, 1H), 7.03 (dd, J=8.79, 2.34 Hz, 1H), 6.88 (d, J=2.34 Hz, 1H), 3.93 (s, 3H). MS (ESI) m/z=285 [M+H]⁺.

### Step 2 4-Methoxy-2-(4,4,5,5-tetramethyl-[1,3,2]dioxaborolan-2-yl)-benzaldehyde

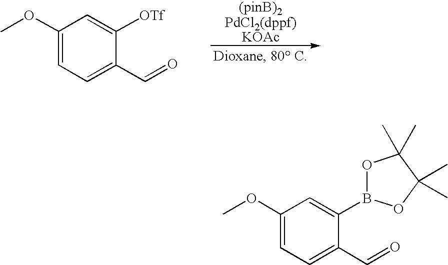

To a solution of bis(pinacolato)diborane (58.66 g, 0.231 mol) in dioxane (600 mL) was added KOAc (52.33 g, 0.533 mol). After degassed for 15 min with nitrogen, PdCl₂(dppf) (13.0 g, 0.0178 mol) and trifluoro-methanesulfonic acid 2-formyl-5-methoxy-phenyl ester (50.51 g, 0.178 mol) were added to the reaction mixture. The mixture was stirred at 80° C. for 45 min. The reaction was quenched by adding ice-water (400 mL). The resulting mixture was extract with 50% EtOAc/hexanes (2×600 mL). The extract was washed with brine, dried and concentrated to dryness. The residue was purified by chromatography on silica gel (EtOAc/hexanes=1:3) to give 43.48 g (93.2% yield) of product as pale-yellow waxy solid. ¹H NMR (400 MHz, CDCl₃) δ 10.88 (s, 1H), 8.40-8.48 (m, 1H), 8.44 (d, J=8.50 Hz, 1H), 7.80 (d, J=2.64 Hz, 1H), 7.54 (dd, J=8.50, 2.64 Hz, 1H), 4.41 (s, 3H), 1.91 (s, 12H). MS (ESI) m/z=263 [M+H]⁺.

### Step 3 6-Methoxy-3H-benzo[c][1,2]oxaborol-1-ol

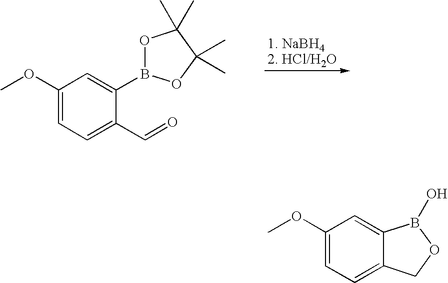

To a solution of 4-methoxy-2-(4,4,5,5-tetramethyl-[1,3,2]dioxaborolan-2-yl)-benzaldehyde (25.0 g, 95.4 mmol) in methanol (160 mL) was slowly added NaBH₄ powder (10.82 g, 0.286 mol) at 0-10° C. After stirred for 1 h at room temperature, the mixture was concentrated to remove one-third of methanol. The resulting mixture was cooled to 0° C., acidified to pH 3 using diluted hydrochloric acid and diluted to two fold with cold water. The white precipitate was collected, washed with 30% MeOH/H₂O, water, and dried to give 11.5 g (73.5% yield) of product as white solid. ¹H NMR (400 MHz, DMSO-d₆) δ 9.11 (s, 1H), 7.29 (d, J=8.21 Hz, 1H), 7.23 (d, J=2.34 Hz, 1H), 7.03 (dd, J=8.21, 2.34 Hz, 1H), 4.90 (s, 2H), 3.75 (s, 3H).

### E2. 3H-Benzo[c][1,2]oxaborole-1,6-diol

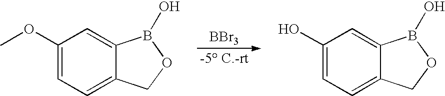

To a solution of 6-methoxy-3H-benzo[c][1,2]oxaborol-1-ol (10.0 g, 61.0 mmol) in dichloromethane (400 mL) was slowly added boron tribromide (134 mL, 1 M in DCM, 0.134 mol) at −10 to −5° C. The mixture was stirred at 0° C. to room temperature for 3 h. The reaction mixture was poured into ice-water (300 mL). The resulting mixture was extract with EtOAc (600 mL). The extract was washed with brine, dried and concentrated to dryness to give 9.11 g (99.6% yield) of product as off-white foam. ¹H NMR (400 MHz, DMSO-d₆) δ 9.27 (br. s., 1H), 9.03 (br. s., 1H), 7.16 (d, J=8.20 Hz, 1H), 7.08 (d, J=2.34 Hz, 1H), 6.86 (dd, J=8.20, 2.34 Hz, 1H). MS (ESI) m/z=151 [M+H]⁺.

### E3. 6-(3-Methoxy-phenoxy)-3H-benzo[c]oxaborol-1-ol

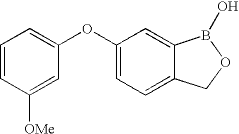

### Step 1. 2-Bromo-4-(3-methoxy-phenoxy)-benzaldehyde

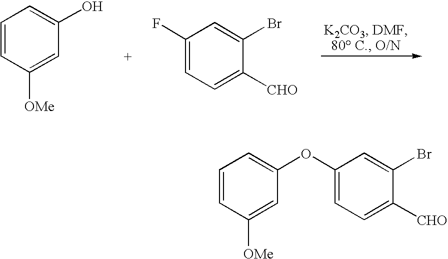

A mixture of 3-methoxy-phenol (5.00 g, 40.32 mmol), 2-bromo-4-fluoro-benzaldehyde (8.18 g, 40.32 mmol) and K₂CO₃ (8.34 g, 60.48 mmol) in DMF (40 mL) was heated at 80° C. for 16 h, cooled to RT, diluted with water (100 mL), the solid formed was collected and washed with water, dried to give compound 2-bromo-4-(3-methoxy-phenoxy)-benzaldehyde (11.3 g, 91% yield) as a brown solid. ¹HNMR (400 MHz, CDCl₃) δ 10.25 (s, 1H), 7.90 (d, J=7.2 Hz, 1H), 7.35 (m, 1H), 7.20 (s, 1H), 7.00 (d, J=7.1 Hz, 1H), 6.81 (m, 1H), 6.70-6.60 (m, 2H), 3.80 (s, 3H).

### Step 2. 4-(3-Methoxy-phenoxy)-2-(4,4,5,5-tetramethyl-[1,3,2]dioxaborolan-2-yl)benzaldehyde

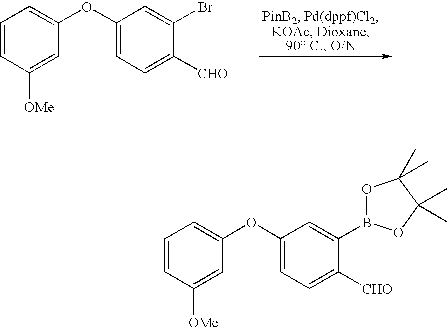

A mixture of 2-bromo-4-(3-methoxy-phenoxy)-benzaldehyde (2.40 g, 7.82 mmol), bis(pinacolato)diborane (2.98 g, 11.73 mmol) Pd(dppf)Cl₂ (0.57 g, 0.78 mmol) and KOAc (2.30 g, 23.46 mmol) in dioxane (30 mL) was degassed for 10 min, heated at 90° C. for 2 h, diluted with EtOAc (100 mL), filtered through a pad of Celite and concentrated. The residue was purified by chromatography to give compound 4-(3-methoxy-phenoxy)-2-(4,4,5,5-tetramethyl-[1,3,2]dioxaborolan-2-yl)benzaldehyde (1.90 g, 68% yield) as an off-yellow oil. ¹HNMR (400 MHz, CDCl₃) δ 10.40 (s, 1H), 7.93 (d, J=8.1 Hz, 1H), 7.44 (s, 1H), 7.25 (m, 1H), 7.03 (m, 1H), 6.75 (m, 1H), 6.60 (m, 2H), 3.80 (s, 3H), 1.40 (s, 12H).

### Step 3. 6-(3-Methoxy-phenoxy)-3H-benzo[c]oxaborol-1-ol

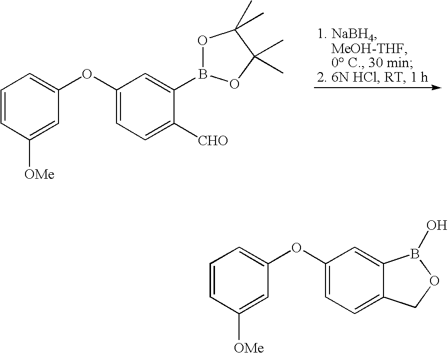

To a cooled (0° C.) solution of 4-(3-methoxy-phenoxy)-2-(4,4,5,5-tetramethyl-[1,3,2]-dioxaborolan-2-yl)benzaldehyde (3.2 g, 9.01 mmol) in MeOH (20 mL) and THF (20 mL) was added NaBH₄ (0.75 g, 19.83 mol) in portions. After the addition was over, the mixture was stirred at 0° C. for 30 min, quenched with 6 N HCl until pH 3, stirred at pH 3 for 20 min, neutralized with NaHCO₃, extracted with dichloromethane, dried and concentrated. The residue was purified by chromatography to give 6-(3-methoxy-phenoxy)-3H-benzo[c]oxaborol-1-ol (1.6 g, 69% yield) as an oil. ¹H NMR (DMSO-d₆, 400 MHz) δ 9.17 (s, 1H), 7.40 (d, J=8.2 Hz, 1H), 7.30-7.20 (m, 2H), 7.18 (m, 1H), 6.71 (m, 1H), 6.56 (s, 1H), 6.50 (m, 1H), 4.98 (s, 2H), 3.70 (s, 3H). MS (ESI) m/z=257 [M+1]⁺.

### E4. 6-(3-Benzyloxy-phenoxy)-3H-benzo[c][1,2]oxaborol-1-ol

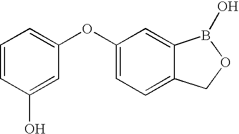

### Step 1. 4-(3-Benzyloxy-phenoxy)-2-bromo-benzaldehyde

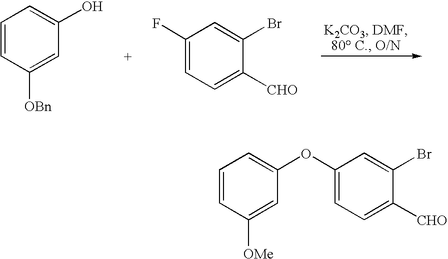

A mixture of 3-benzyloxy-phenol (5.00 g, 23.81 mmol), 2-bromo-4-fluoro-benzaldehyde (4.84 g, 25 mmol) and K₂CO₃ (5.18 g, 37.5 mmol) in DMF (30 mL) was heated at 80° C. for 16 h, cooled to RT, diluted with EtOAc, filtered through a pad of Celite and concentrated. The residue was purified by chromatography to give compound 4-(3-benzyloxy-phenoxy)-2-bromo-benzaldehyde (7.7 g, 100% yield). ¹HNMR (400 MHz, CDCl₃) δ 10.23 (s, 1H), 7.90 (d, J=2.4 Hz, 1H), 7.50-7.30 (m, 6H), 7.20 (s, 1H), 6.98 (m, 1H), 6.85 (m, 1H), 6.70 (m, 2H), 5.03 (s, 2H).

### Step 2. 4-(3-Benzyloxy-phenoxy)-2-(4,4,5,5-tetramethyl-[1,3,2]dioxaborolan-2-yl)benzaldehyde

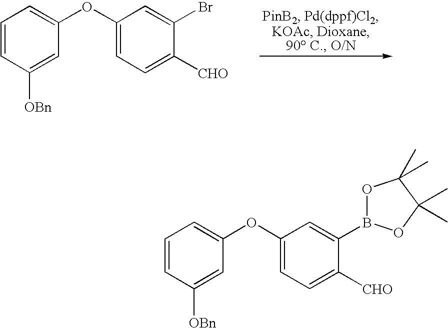

A mixture of 4-(3-benzyloxy-phenoxy)-2-bromo-benzaldehyde (4.30 g, 13.30 mmol), bis(pinacolato)diborane (5.07 g, 19.97 mmol), Pd(dppf)Cl₂ (0.97 g, 1.33 mmol) and KOAc (3.91 g, 39.90 mmol) in dioxane (40 mL) was degassed for 10 min, heated at 90° C. for 2 h, diluted with EtOAc (100 mL), filtered through a pad of Celite and concentrated. The residue was purified by chromatography to give 4-(3-benzyloxy-phenoxy)-2-(4,4,5,5-tetramethyl-[1,3,2]dioxaborolan-2-yl)benzaldehyde (4.31 g, 87% yield) as an off-white solid. ¹HNMR (400 MHz, CDCl₃) δ 10.40 (s, 1H), 7.92 (d, J=2.4 Hz, 1H), 7.50-7.20 (m, 8H), 7.03 (m, 1H), 6.80 (m, 1H), 6.70-6.60 (m, 1H), 5.03 (s, 2H), 1.40 (s, 12H).

### Step 3. 6-(3-Benzyloxy-phenoxy)-3H-benzo[c][1,2]oxaborol-1-ol

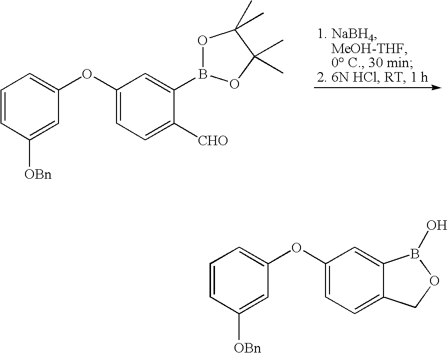

To a cooled (0° C.) solution of 4-(3-benzyloxy-phenoxy)-2-(4,4,5,5-tetramethyl-[1,3,2]dioxaborolan-2-yl)benzaldehyde (2.08 g, 2.18 mmol) in MeOH (10 mL) and THF (8 mL) was added NaBH₄ (0.47 g, 12.33 mol) in portions. After the addition was over, the mixture was stirred at 0° C. for 10 min, quenched with 3 N HCl until pH 3, stirred at pH 3 for 20 min, extracted with EtOAc, dried and concentrated. The residue was recrystallized from dichloromethane and hexane to give 6-(3-benzyloxy-phenoxy)-3H-benzo[c][1,2]oxaborol-1-ol (660 mg, 35% yield). Mp 168-170° C. ¹H NMR (400 MHz, DMSO-d₆) δ 9.20 (s, 1H), 7.50-7.23 (m, 8H), 7.20 (m, 1H), 6.80 (m, 1H), 6.63 (s, 1H), 6.52 (m, 1H), 5.10 (s, 2H), 4.98 (s, 2H). MS (ESI) m/z=333 [M+1]⁺

### E5. 6-(3-Hydroxy-phenoxy)-3H-benzo[c][1,2]oxaborol-1-ol

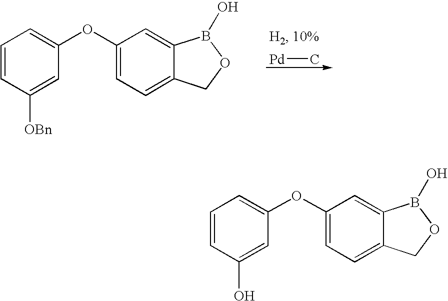

A solution of 6-(3-benzyloxy-phenoxy)-3H-benzo[c][1,2]oxaborol-1-ol (260 mg) in EtOAc was hydrogenated under 50 psi with H₂ in the presence of 10% Pd—C (50 mg) for 2 h, filtered through a pad of Celite and concentrated. The residue was purified by prep-HPLC to give 6-(3-hydroxy-phenoxy)-3H-benzo[c][1,2]oxaborol-1-ol (40 mg). Mp 159-161° C. ¹H NMR (DMSO-d₆, 400 MHz) δ 9.50 (br s, 1H), 9.17 (s, 1H), 7.49 (m, 1H), 7.27 (s, 1H), 7.10 (m, 2H), 6.50 (d, J=6.7 Hz, 1H), 6.40 (d, J=6.6 Hz, 1H), 6.30 (s, 1H), 4.96 (s, 2H). MS (ESI) m/z=243 [M+H]⁺.

### E6. 3-(1-Hydroxy-1,3-dihydro-benzo[c][1,2]oxaborol-6-yloxy)-benzoic acid methyl ester

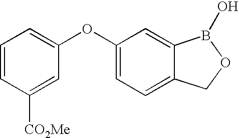

### Step 1. 3-(3-Bromo-4-formyl-phenoxy)-benzoic acid methyl ester

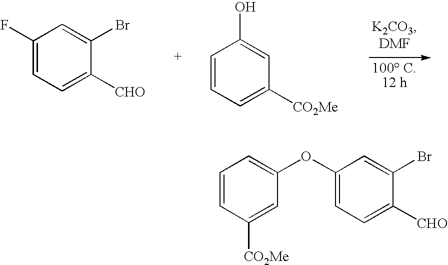

A stirred solution of 2-bromo-4-fluorobenzaldehyde (5.0 g, 24.6 mmol), methyl 3-hydroxybenzoate (3.74 g , 24.6 mmol) and K₂CO₃ (5.09 g, 36.9 mmol) in DMF (40 mL) was heated at 100° C. for 12 h. The reaction mixture was cooled to room temperature and water (100 mL) was added, extracted with EtOAc (3×25 mL), dried over MgSO₄, concentrated and column chromatographed over silica gel afforded 3-(3-bromo-4-formyl-phenoxy)-benzoic acid methyl ester (7.8 g, 94% yield). ¹H NMR (400 MHz, CDCl₃) δ 10.26 (s, 1H), 7.95-7.90 (m, 2H), 7.75-7.74 (m, 1H), 7.52 (t, J=8 Hz, 1H), 7.30 (dd, J=2.8, 8 Hz, 1H), 7.17 (d, J=2.4 Hz, 1H), 6.98 (dd, J=4, 12 Hz, 1H), 3.92 (s, 3H). MS (ESI) m/z=335 [M+H]⁺.

### Step 2. 3-[4-Formyl-3-(4,4,5,5-tetramethyl-[1,3,2]dioxaborolan-2-yl)-phenoxy]-benzoic acid methyl ester

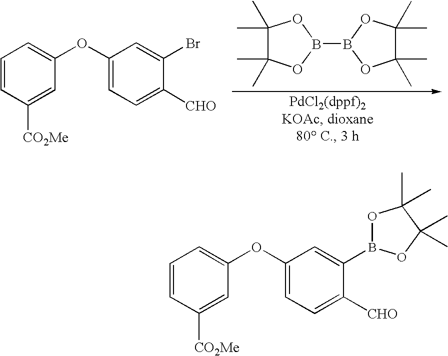

A solution of 3-(3-bromo-4-formyl-phenoxy)-benzoic acid methyl ester (3.0 g, 8.95 mmol) in anhydrous 1,4-dioxane (100 mL) was degassed for 15 minutes, to this was added bis(pinacolato)diborane (4.54 g, 17.9 mmol), PdCl₂(dppf) (0.65 g, 0.89 mmol), KOAc (2.63 g, 26.8 mmol) and the resulting solution was warmed at 80° C. for 3 h. The reaction mixture was then cooled and filtered through a Celite pad. The filtrate was evaporated, and the residue was dissolved in EtOAc (100 mL), washed with water (2×20 mL), dried, concentrated and purified by column chromatography over silica gel to provide 3-[4-Formyl-3-(4,4,5,5-tetramethyl-[1,3,2]dioxaborolan-2-yl)-phenoxy]-benzoic acid methyl ester (2.0 g, 59% yield). ¹H NMR (400 MHz, CDCl₃) δ 10.42 (s, 1H), 7.95 (d, J=8.8 Hz, 1H), 7.87 (d, J=8 Hz, 1H), 7.70 (t, J=2 Hz, 1H), 7.48-7.43 (m, 2H), 7.24 (dd, J=4, 8 Hz, 1H), 7.05 (dd, J=4, 10 Hz, 1H), 3.91 (s, 3H), 1.38 (s, 12H). MS (ESI) m/z=383 [M+H]⁺.

### Step 3. 3-(1-Hydroxy-1,3-dihydro-benzo[c][1,2]oxaborol-6-yloxy)-benzoic acid methyl ester

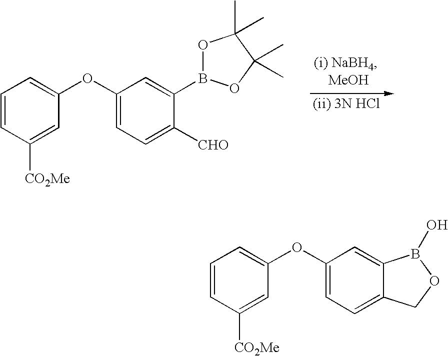

To a stirred solution of 3-[4-formyl-3-(4,4,5,5-tetramethyl-[1,3,2]dioxaborolan-2-yl)-phenoxy]-benzoic acid methyl ester (0.48 g, 1.26 mmol) in MeOH (10 mL) at 0° C. was added NaBH₄ (0.024 g, 0.63 mmol) and the resulting solution was stirred for 2 h slowly warming to room temperature. Solvent was concentrated to 2 mL and 5 mL of 3N HCl was added at 0° C. and stirred for 16 h. Volatiles were evaporated off and the residue was extracted with EtOAc (2×10 mL), washed with water (10 mL), dried over MgSO₄ and purified by column chromatography over silica gel furnished 3-(1-hydroxy-1,3-dihydro-benzo[c][1,2]oxaborol-6-yloxy)-benzoic acid methyl ester (0.2 g, 55% yield) as a viscous oil. ¹H NMR (400 MHz, CDCl₃) δ 9.21 (s, 1H), 7.69 (dd, J=1.2, 5.4 Hz, 1H), 7.53 (t, J=8 Hz, 1H), 7.46-7.39 (m, 2H), 7.33-7.31 (m, 2H), 7.21 (dd, J=2.4, 8.4 Hz, 1H), 4.97 (s, 2H), 3.80 (s, 3H). MS (ESI) m/z=285 [M+H]⁺.

### E7. 3-(1-Hydroxy-1,3-dihydro-benzo[c][1,2]oxaborol-6-yloxy)-benzoic acid

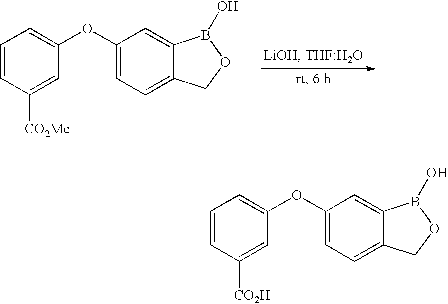

To a stirred solution of 3-(1-hydroxy-1,3-dihydro-benzo[c][1,2]oxaborol-6-yloxy)-benzoic acid methyl ester (0.085 g, 0.29 mmol) in THF:H₂O (10 mL, 5:1) at room temperature was added LiOH and the reaction mixture was stirred for 12 h at room temperature. Acidified to pH 3 with 6 N HCl, extracted with EtOAc (2×10 mL), washed with water, dried and purified by column chromatography over silica gel furnished 3-(1-Hydroxy-1,3-dihydro-benzo[c][1,2]oxaborol-6-yloxy)-benzoic acid (0.075 g, 93% yield) as a white solid. Mp 192-194° C. ¹H NMR (400 MHz, CDCl₃) δ 9.25 (s, 1H), 7.70 (d, J=8 Hz, 1H), 7.54-7.47 (m, 2H), 7.38 (dd, J=2, 16.4 Hz, 2H), 7.30 (dd, J=2, 7.8 Hz, 1H), 7.24 (dd, J=2, 8 Hz, 1H), 5.0 (s, 2H). MS (ESI) m/z=269 [M−H]⁻.

### E8. Ethyl 4-(1-hydroxy-1,3-dihydrobenzo[c][1,2]oxaborol-6-yloxy)benzoate

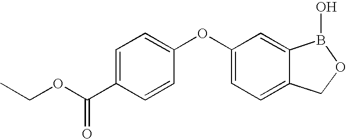

### Step 1. Ethyl 4-(3-bromo-4-formylphenoxy)benzoate

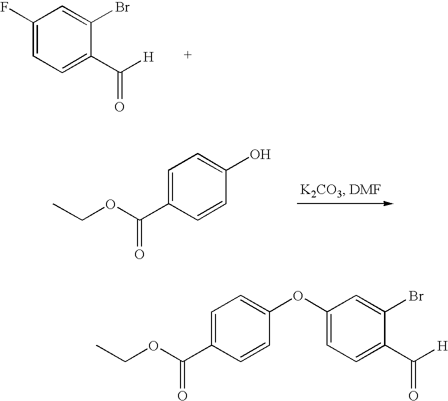

Bromo-4-fluoro benzaldehyde 10 g (49.26 mmol) and Ethyl 4-hydroxybenzoate 8.19 (49.26 mmol) were mixed in a solution of DMF (50 mL). To this was added potassium carbonate 10.21 g (73.89 mmol). The reaction mixture was stirred at 100° C. (oil bath) for 17 hour under N₂. Cooled to room temperature, mixture of EtOAc and water was added. Stirred for 30 min, concentrated via rotary evaporation to remove most of organic solvent. Filtered, washed with water, dried to get the target molecule, 17 g (98.8% yield) as white solid. ¹H NMR (DMSO-d₆, 300 MHz) δ ppm 10.13 (s, 1H), 8.03 (d, 2H, J=8.7 Hz) , 7.89 (d, 1H, J=8.7 Hz), 7.45 (m, 1H), 7.26 (d, 2H, J=8.7 Hz), 7.19 (m, 1H), 4.30 (q, 2H) and 1.30 (t, 3H).

### Step 2. Ethyl 4-(4-formyl-3-(4,4,5,5-tetramethyl-1,3,2-dioxaborolan-2-yl)phenoxy)benzoate

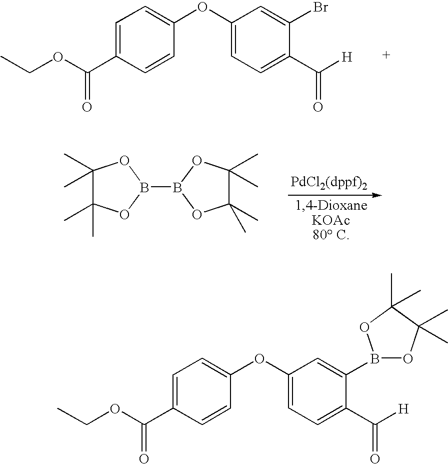

To a solution of ethyl 4-(3-bromo-4-formylphenoxy)benzoate (6.98 g, 20 mmol), KOAc (5.88 g, 600 mmol), bis(pinacolato)diboron (6.10 g, 24 mmol,) in anhydrous 1,4-dioxane (80 mL) was added PdCl₂(dppf)₂ (408 mg; 2.5 mol %). The reaction mixture was degassed with N₂, and then heated at 80° C. with magnetic stirring. The reaction was monitored with TLC and was completed after 8 hours. The mixture was cooled to room temperature, filtered through celite and washed with ethyl acetate and then evaporated. The residue was dissolved in minimum EtOAc and passed through a very short but big silica gel column eluted with a mixed solvent of hexane:EtOAc (3:1, v/v) to remove dark color giving a light yellow oil. Chromatography on silica gel again (Hexane/EtOAc 7:3). The product was collected and concentrated as colorless oil 9 g (100% yield). ¹H NMR (DMSO-d₆, 300 MHz) δ ppm 10.24 (s, 1H), 7.99 (m, 3H), 7.29 (m, 2H), 7.17 (d, J=9 Hz, 2H), 4.30 (q, 2H), 1.29 (s, 12H) and 1.31 (t, 3H).

### Step 3. Ethyl 4-(1-hydroxy-1,3-dihydrobenzo[c][1,2]oxaborol-6-yloxy)benzoate

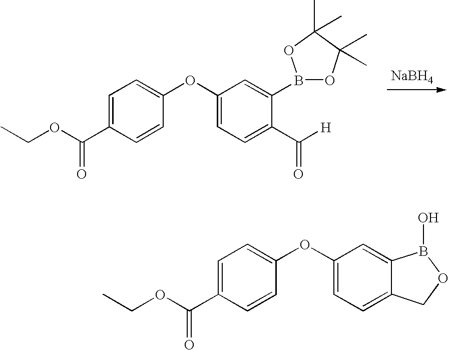

To a solution of ethyl 4-(4-formyl-3-(4,4,5,5-tetramethyl-1,3,2-dioxaborolan-2-yl)phenoxy)benzoate in MeOH was added NaBH₄ portion wise at 0° C. Stirred at 0° C. to rt for 6 hr. Added 3N HCl aqueous solution till pH=2. Concentrated by rota vapor to remove solvent. No solid precipitated out. Extracted with EtOAc, dried, and concentrated. Chromatography (Hexane/EtOAc) was employed to get the target molecule. ¹H NMR (DMSO-d₆, 300 MHz) δ ppm 9.21 (s, 1H), 7.94 (d, J=8.1 Hz, 2H), 7.48 (d, J=8.1 Hz, 1H), 7.38 (d, J=2.4 Hz, 1H), 7.25 (dd, J=8.1, 2.4 Hz, 1H), 7.03 (d, J=7.8 Hz, 2H), 4.99 (s, 2H), 4.27 (t, 2H) and 1.29 (t, 3H). MS (ESI) m/z 269.1 [M−H]⁻.

### E9. 4-(1-Hydroxy-1,3-dihydrobenzo[c][1,2]oxaborol-6-yloxy)benzoic acid

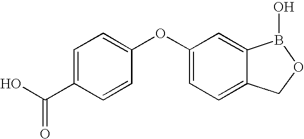

E9 was synthesized using the similar procedure in E55 using ethyl 4-(1-hydroxy-1,3-dihydrobenzo[c][1,2]oxaborol-6-yloxy)benzoate as starting material. Mp 197-200° C. ¹H NMR (DMSO-d₆, 300 MHz) δ 12.83 (s, 1H), 9.22 (s, 1H), 7.93 (m, 2H), 7.48 (d, 1H, J=8.4 Hz), 7.38 (d, 1H, J=2.1 Hz), 7.25 (dd, 1H, J=2.1, 7.8 Hz), 7.01 (m, 1H), 4.98 (s, 2H). MS (ESI) m/z 269.1 [M−H]⁻.

### E10. 6-(3-Hydroxymethyl-phenoxy)-3H-benzo[c][1,2]oxaborol-1-ol

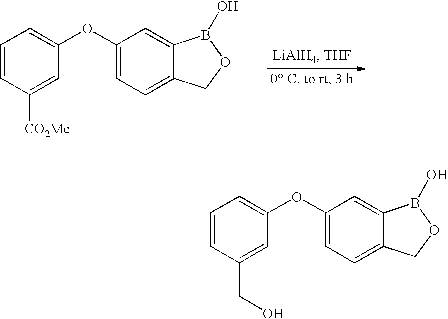

To a stirred solution of 3-(1-hydroxy-1,3-dihydro-benzo[c][1,2]oxaborol-6-yloxy)-benzoic acid methyl ester (0.18 g, 0.63 mmol) in THF (10 mL) at 0° C. was added LiAlH₄ (0.036 g, 0.95 mmol) and the reaction mixture was stirred for 3 h slowly warming to room temperature. The reaction was quenched by the addition of 1N HCl (5 mL) at 0° C., extracted with EtOAc (2×10 mL), washed with water, dried and purified by column chromatography over silica gel furnished 6-(3-hydroxymethyl-phenoxy)-3H-benzo[c][1,2]oxaborol-1-ol (0.14 g, 86% yield) as a white solid. Mp: 256-258° C. ¹H NMR (400 MHz, CDCl₃) δ 9.2 (s, 1H), 7.41 (d, J=8 Hz, 1H), 7.31-7.26 (m, 2H), 7.15 (dd, J=2.4, 8.4 Hz, 1H), 7.0 (d, J=8 Hz, 1H), 6.91-6.84 (m, 2H), 5.23 (t, J=6 Hz, 1H), 4.95 (s, 2H), 4.45 (d, J=6 Hz, 2H). MS (ESI) m/z=255 [M−H]⁻.

### E11. 6-(3-Nitro-phenoxy)-3H-benzo[c][1,2]oxaborol-1-ol

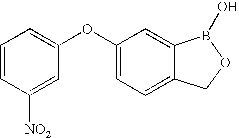

### Step 1. 2-Bromo-4-(3-nitro-phenoxy)-benzaldehyde

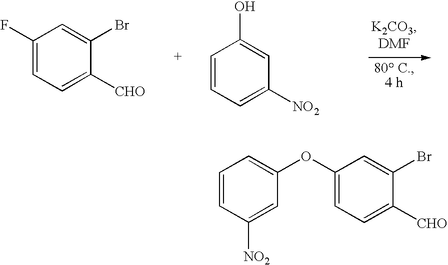

An orange suspension of 2-bromo-4-fluoro-benzaldehyde (5.00 g, 24.62 mmol), 3-nitro-phenol (3.76 g, 27.02 mmol) and potassium carbonate (5.10 g, 36.90 mmol) in N,N′-dimethylformamide (20 mL) was heated at 80° C. for 4 hours. The mixture was cooled to room temperature, then diluted with water and extracted with ethyl acetate. The extracts were dried (Na₂SO₄), filtered, and concentrated to afford 2-bromo-4-(3-nitro-phenoxy)-benzaldehyde (7.9 g, crude, quantitative), as a light beige solid. ¹H NMR (400 MHz, CDCl₃) δ 10.29 (s, 1H), 8.12 (d, J=8.21 Hz, 1H), 7.98-7.92 (m, 2H), 7.62 (t, J=8.21 Hz, 1H), 7.42 (dd, J=8.21, 1.95 Hz, 1H), 7.25 (d, J=2.35 Hz, 1H), 7.05 (dd, J=8.60, 2.35 Hz, 1H).

### Step 2. 4-(3-Nitro-phenoxy)-2-(4,4,5,5-tetramethyl-[1,3,2]dioxaborolan-2-yl)-benzaldehyde

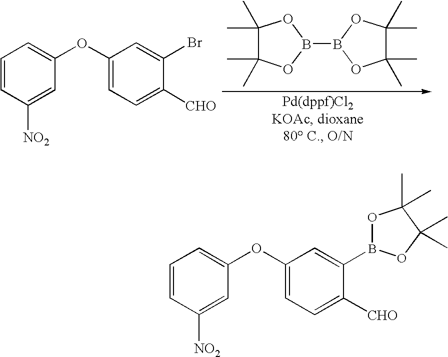

A yellow suspension of 2-bromo-4-(3-nitro-phenoxy)-benzaldehyde (3.00 g, 9.31 mmol), bis(pinacolato)diborane (3.55 g, 13.98 mmol), and potassium acetate (2.74 g, 27.93 mmol) in 1,4-dioxane (30 mL) was degassed with nitrogen gas for 15 minutes, then treated with [1,1′-bis(diphenylphosphino)ferrocene]palladium(II) chloride 1:1 complex with dichloro-methane (1.36 g, 1.86 mmol). The resulting mixture was heated at 80° C. overnight. The mixture was cooled to room temperature, then diluted with ethyl acetate. The suspension was filtered through a pad of Celite and the pad was washed with ethyl acetate. The filtrate was concentrated to a brown residue, then purified by column chromatography (silica gel, 10-20% ethyl acetate/hexanes gradient elution) to afford 4-(3-nitro-phenoxy)-2-(4,4,5,5-tetramethyl-[1,3,2]dioxaborolan-2-yl)-benzaldehyde (2.70 g, 79% yield) as a light beige solid. ¹H NMR (400 MHz, CDCl₃) δ 10.49 (s, 1H), 8.06-8.00 (m, 2H), 7.87 (br s, 1H), 7.55 (t, J=8.21 Hz, 1H), 7.49 (d, J=2.35 Hz, 1H), 7.37 (d, J=7.03 Hz, 1H), 7.15 (dd, J=8.21, 2.35 Hz, 1H), 1.39 (s, 12H).

### Step 3. 6-(3-Nitro-phenoxy)-3H-benzo[c][1,2]oxaborol-1-ol

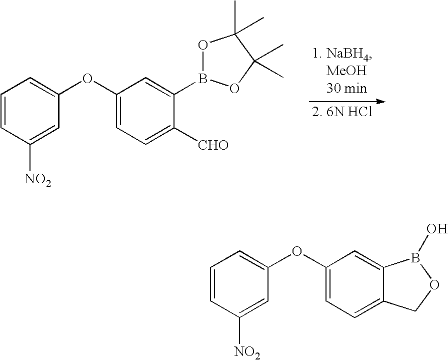

An ice-cold light yellow solution of 4-(3-nitro-phenoxy)-2-(4,4,5,5-tetramethyl-[1,3,2]dioxaborolan-2-yl)-benzaldehyde, (2.70 g, 7.30 mmol) in methanol (20 mL) was treated with sodium borohydride (0.28 g, 7.40 mmol) in portions. The mixture was stirred for 30 minutes, then quenched with 6 M HCl. The mixture was stirred for 30 minutes then extracted with ethyl acetate. The organic extracts were dried (Na₂SO₄), filtered, and concentrated in vacuum to give a yellow oil. The residue was absorbed onto silica gel then loaded onto a column and eluted with hexanes/ethyl acetate (5:1 to 1:2 gradient) to afford a white foam. The foam was triturated with methanol and water. The white solid was collected by vacuum filtration and dried under high vacuum to afford 6-(3-nitro-phenoxy)-3H-benzo[c][1,2]oxaborol-1-ol (1.11 g, 56% yield). ¹H NMR (400 MHz, DMSO-d₆) δ 9.24 (s, 1H), 7.99 (d, J=8.21 Hz, 1H), 7.71-7.65 (m, 2H), 7.54-7.47 (m, 2H), 7.42 (d, J=2.35 Hz, 1H), 7.30 (dd, J=8.21, 2.34 Hz, 1H), 5.02 (s, 2H).

### E12. 6-(3-Amino-phenoxy)-3H-benzo[c][1,2]oxaborol-1-ol

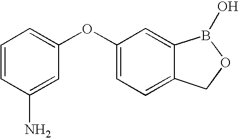

### 6-(3-Amino-phenoxy)-3H-benzo[c][1,2]oxaborol-1-ol

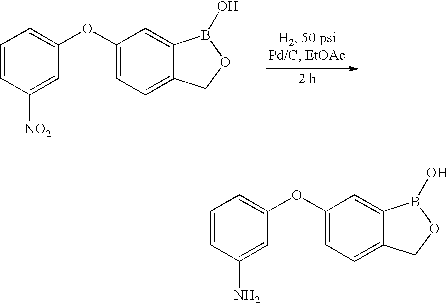

A colorless solution of 6-(3-nitro-phenoxy)-3H-benzo[c][1,2]oxaborol-1-ol (1.11 g, 4.10 mmol) in ethyl acetate (60 mL) was treated with palladium (10% wet on charcoal, 0.28 g), then hydrogenation at 50 psi hydrogen gas for 2 hours. The mixture was filtered through a Celite pad and rinsed with ethyl acetate. The filtrate was concentrated to afford a white foam. The foam was triturated with methanol and water to afford 6-(3-amino-phenoxy)-3H-benzo[c][1,2]oxaborol-1-ol (0.74 g, 76% yield), as a white solid. Mp 142-143° C. ¹H NMR (400 MHz, DMSO-d₆) δ 9.18 (s, 1H), 7.40 (d, J=8.21 Hz, 1H), 7.30 (d, J=2.35 Hz, 1H), 7.15 (dd, J=8.21, 2.35 Hz, 1H), 6.99 (t, J=7.82 Hz, 1H), 6.31 (dd, J=7.82, 1.17 Hz, 1H), 6.18-6.10 (m, 2H), 5.21 (s, 2H), 4.96 (s, 2H); MS (ESI) m/z=242 [M+H]⁺.

### E13. 6-(4-Nitrophenoxy)benzo[c][1,2]oxaborol-1(3H)-ol

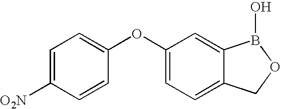

### Step 1. 4-(4-Nitrophenoxy)-2-(4,4,5,5-tetramethyl-1,3,2-dioxaborolan-2-yl)benzaldehyde

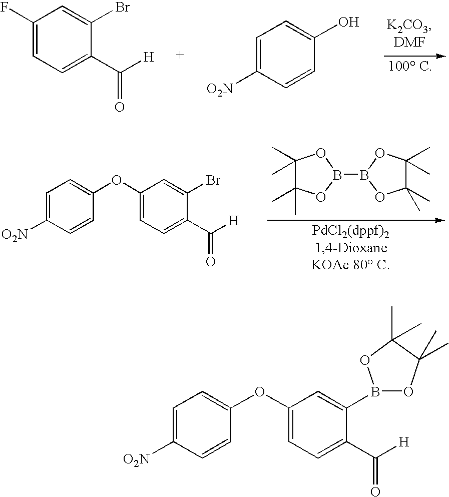

This was made according to the procedure for 4-phenoxy-2-(4,4,5,5-tetramethyl-1,3,2-dioxaborolan-2-yl)benzaldehyde with the exception of using 4-nitrophenol instead of phenol as starting material. ¹H NMR (DMSO-d₆, 300 MHz) δ ppm 10.26 (s, 1H) 8.29 (d, J=9 Hz, 2H) 8.01 (d, J=7.8 Hz, 1H) 7.40-7.37 (m, 2H) 7.25 (d, J=9 Hz, 2H) 1.31 (s, 12H).

### Step 2. 6-(4-Nitrophenoxy)benzo[c][1,2]oxaborol-1(3H)-ol

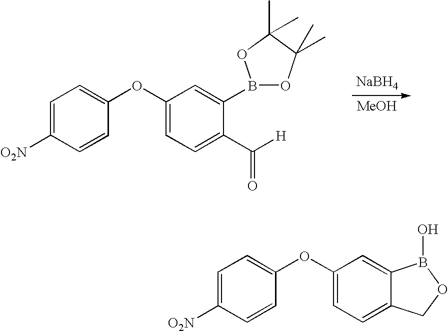

To a suspension of 4-(4-nitrophenoxy)-2-(4,4,5,5-tetramethyl-1,3,2-dioxaborolan-2-yl)benzaldehyde (2 g, 5.42 mmol) in 10 ml MeOH in ice bath was added NaBH₄ (288 mg, 7.59 mmol) potion wise. After stirring at room temperature for 1 hour, the reaction was quenched with water, acidified with 1N HCl until pH 3 then extracted with ethyl acetate. The combined organic layer was dried over Na₂SO₄, filtered and evaporated under reduced pressure to afford a light yellow solid. Product was recrystallized from acetone and water as a white powder (460 mg, 31% yield). MS (ESI) m/z 270 [M−H]⁻.

### E14. 6-(4-Aminophenoxy)benzo[c][1,2]oxaborol-1(3H)-ol

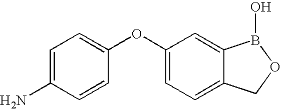

### 6-(4-Aminophenoxy)benzo[c][1,2]oxaborol-1(3H)-ol

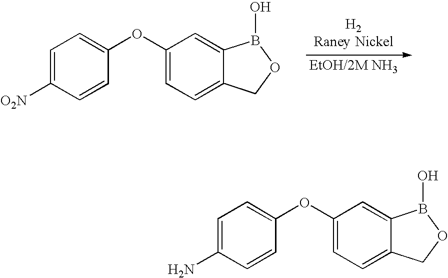

6-(4-nitrophenoxy)benzo[c][1,2]oxaborol-1(3H)-ol (197 mg, 0.73 mmol)was dissolved in a mixture of 10 ml MeOH and 2M EtOH, then about 1.5 ml of Raney Nickel slurry in water was added. This was subjected to hydrogenation (45 psi) for 4 hours on a Parr-Shaker. The mixture was then filtered through Celite and the filtrate was concentrated. The residue was purified by column to give the title compound as an off-white solid (108 mg, 62% yield). ¹H NMR (DMSO-d₆, 300 MHz) δ ppm 9.11 (s, 1H), 7.32 (d, J=8.40 Hz, 1H), 7.12 (d, J=2.40 Hz,1H), 7.20 (dd, J=8.40, 2.7 Hz, 1H), 6.74 (d, J=6.30 Hz, 2H), 6.57 (d, J=6.6 Hz, 2H), 4.96 (s, 2H), 4.90 (s, 2H). MS (ESI) m/z 242 [M+H]⁺.

### E15. (3-(1-Hydroxy-1,3-dihydrobenzo[c][1,2]oxaborol-6-yloxy)phenyl)methanaminium chloride

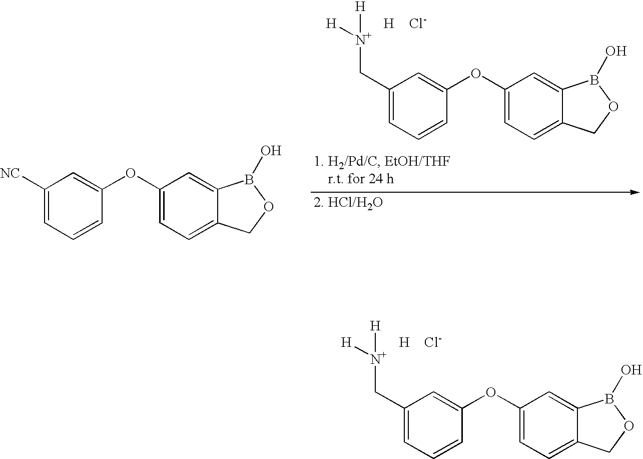

The title compound was synthesized by the same procedure as described above for the preparation of its para-analog. Yield 59.9%. Mp 180-188° C. ¹H NMR (DMSO-d₆, 300 MHz): δ 9.23 (br. s, 1H), 8.38 (br. s, 3H), 7.44 (d, J=8.4 Hz, 2H), 7.40 (t, J=8.4 Hz, 1H), 7.35 (d, J=2.4 Hz, 1H), 7.22 (dm, J=8.1 Hz, 1H), 7.18-7.15 (m, 2H), 6.98 (ddd, J=8.1 & 2.4 & 0.9 Hz, 1H), 4.97 (s, 2H) and 3.99 (br. s, 2H) ppm. Purity (HPLC): 94.4% at 220 nm and 98.5% at 254 nm. MS: m/z=256 (M+1, ESI+) and m/z=254 (M−1, ESI−).

### E16. (4-(1-Hydroxy-1,3-dihydrobenzo[c][1,2]oxaborol-6-yloxy)phenyl)methanaminium chloride

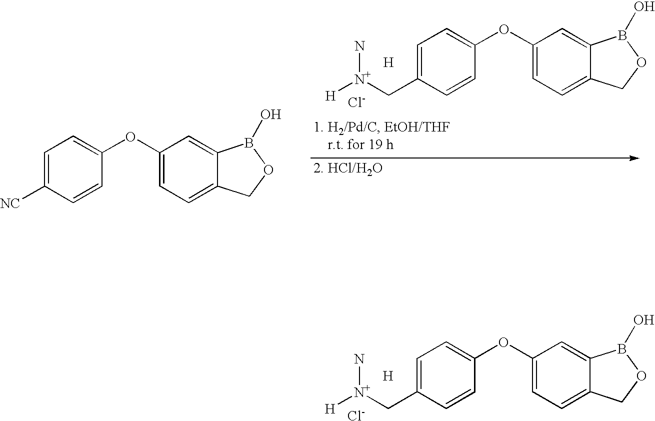

To the solution of 4-(1-hydroxy-1,3-dihydrobenzo[c][1,2]oxaborol-6-yloxy)benzonitrile (6-(4-Cyanophenoxy)-1-hydroxy-2,1-benzoxaborole, 1 g, 3.98 mmol) in a mixed solvent of EtOH (100 mL) and THF (25 mL) under N₂ was added Pd/C (10 wt. %, 0.169 g). The reaction mixture was hydrogenated with a H₂ balloon at room temperature with stirring for 19 h. The mixture was filtered, rotary evaporated and purified by silica gel column eluted with MeOH containing 0.6% v. NH₄OH (3 mL 28-30% NH₄OH to 500 mL MeOH). The white solid obtained was dissolved in water (80 mL) and 6N HCl (2 mL) was added, filtered and the filtrate was freeze-dried to give the desired salt (4-(1-hydroxy-1,3-dihydrobenzo[c][1,2]oxaborol-6-yloxy)phenyl)methanaminium chloride as white solid (0.663 g, 2.27 mmol, yield 57.1%). Mp>230° C. ¹H NMR (DMSO-d₆, 300 MHz): δ 9.23 (br. s, 1H), 8.38 (br. s, 3H), 7.49 (d, J=8.4 Hz, 2H), 7.43 (d, J=8.4 Hz, 1H), 7.31 (d, J=2.4 Hz, 1H), 7.16 (dd, J=8.1 & 2.4 Hz, 1H), 7.02 (d, J=8.4 Hz, 2H), 4.97 (s, 2H) and 3.97 (q, J=5.4 Hz, 2H) ppm. Purity (HPLC): 91.1% at 220 nm and 86.1% at 254 nm. MS: m/z=256 (M+1, ESI+) and m/z=254 (M−1, ESI−).

### E17. 6-(Pyridine-3-yloxyl)-3H-benzo[c][1,2]oxaborol-1-ol

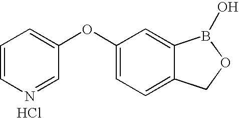

### Step 1. 2-Bromo-4-(pyridine-3-yloxy)-benzaldehyde

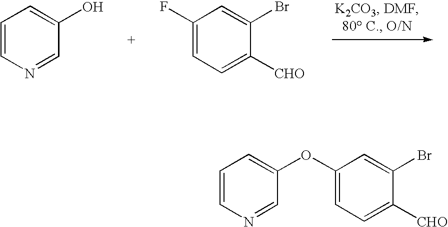

A mixture of pyridin-3-ol (4.18 g, 44 mmol), 2-bromo-4-fluoro-benzaldehyde (8.13 g, 40 mmol) and K₂CO₃ (8.28 g, 60 mmol) in DMF (30 mL) was heated at 80° C. for 16 h, cooled to RT, diluted with water (100 mL), the solid formed was collected and washed with water, dried to give 2-bromo-4-(pyridine-3-yloxy)-benzaldehyde (8.5 g, 100% yield) as a brown solid. ¹HNMR (400 MHz, DMSO-d₆) δ 10.50 (s, 1H), 8.50 (m, 2H), 7.87 (d, 1H), 7.70 (m, 1H), 7.50 (m, 1H), 7.40 (s, 1H), 7.10 (d, 1H).

### Step 2. 4-(Pyridine-3-yloxy)-2-(4,4,5,5-tetramethyl-[1,3,2]dioxaborolan-2-yl)benzaldehyde

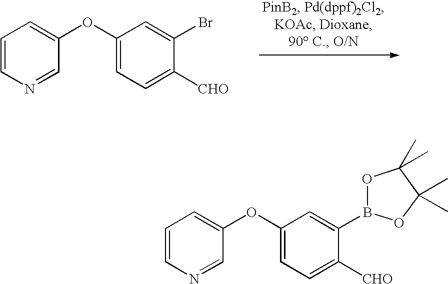

A mixture of 2-bromo-4-(pyridine-3-yloxy)-benzaldehyde (2.08 g, 9.53 mmol), bis(pinacolato)diborane (3.63 g, 14.29 mmol) PdCl₂(dppf) (0.70 g, 0.95 mmol) and KOAc (2.80 g, 28.59 mmol) in dioxane (30 mL) was degassed for 10 min, heated at 90° C. for 2 h, diluted with EtOAc (100 mL), filtered through a pad of Celite and concentrated. The residue was purified by chromatography to give 4-(pyridine-3-yloxy)-2-(4,4,5,5-tetramethyl-[1,3,2]dioxaborolan-2-yl)benzaldehyde (1.91 g, 75% yield) as an off-yellow oil. ¹HNMR (400 MHz, CDCl₃) δ 10.40 (s, 1H), 8.42 (m, 2H), 7.98 (d, 1H), 7.43 (s, 1H), 7.30 (m, 2H), 7.07 (d, 1H), 1.40 (s, 12H).

### Step 3. 6-(Pyridine-3-yloxyl)-3H-benzo[c][1,2]oxaborol-1-ol

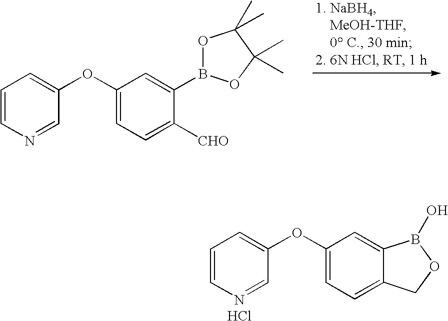

To a cooled (0° C.) solution of 4-(pyridine-3-yloxy)-2-(4,4,5,5-tetramethyl-[1,3,2]dioxaborolan-2-yl)benzaldehyde (0.71 g, 2.18 mmol) in MeOH (6 mL) was added NaBH₄ (0.22 g, 5.87 mol) in portions. After the addition was over, the mixture was stirred at 0° C. for 10 min, quenched with 3 N HCl until pH 3, stirred at pH 3 for 20 min, neutralized with NaHCO₃, extracted with dichloromethane, dried and concentrated. The residue was treated with 1 N HCl (3 mL), concentrated, diluted with THF. The solid was collected to give 6-(pyridine-3-yoxyl)-3H-benzo[c]oxaborol-1-ol (230 mg, 46% yield). Mp 172-174° C. ¹H NMR (400 MHz, DMSO-d₆) δ 8.54 (m, 1H), 8.47 (m, 1H), 7.66 (m, 2H), 7.49 (d, J=8.0 Hz, 1H), 7.39 (d, J=2.4 Hz), 7.28 (m, 1H), 4.99 (s, 2H).; MS (ESI) m/z=228 [M+H]⁺; Elemental Analysis cacld for C₁₂H₁₁BNO₃.HCl.0.1 H₂O: C, 54.33; H, 4.26; N, 5.28. Found: C, 54.02; H, 4.18; N, 5.41.

### E18. 6-(1-Hydroxy-1,3-dihydro-benzo[c][1,2]oxaborol-6-yloxy)-pyridine-2-carboxylic acid

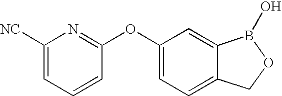

### 6-(1-Hydroxy-1,3-dihydro-benzo[c][1,2]oxaborol-6-yloxy)-pyridine-2-carbonitrile

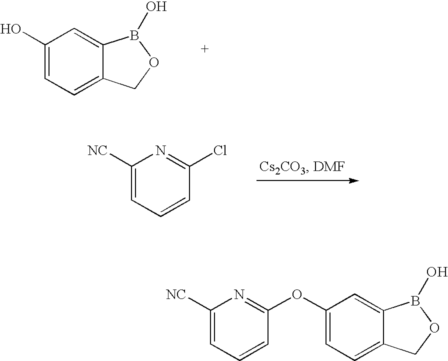

To a solution of 3H-benzo[c][1,2]oxaborole-1,6-diol (2.0 g, 13.33 mmol) in anhydrous DMF (8 mL) were added Cs₂CO₃ (10.86 g, 33.33 mmol) and 6-chloro-pyridine-2-carbonitrile (1.71 g, 14.0 mmol) at room temperature. After stirring at 70° C. for 8 h, the reaction mixture was cooled to 0° C. diluted with water (20 mL) and acidified to pH 3 using diluted hydrochloric acid. The mixture was extracted with EtOAc. The extract was washed with brine and dried to give the crude product which was purified by chromatography on silica gel (DCM/MeOH=40:2) to give 2.20 g of product. ¹HNMR (400 MHz, DMSO-d₆) δ 9.23 (br. s., 1H), 8.08 (m, 1H), 7.78 (d, J=7.33 Hz, 1H), 7.35-7.56 (m, 3H), 7.29 (dd, J=8.20, 2.05 Hz, 1H), 5.01 (s, 2H). MS (ESI) m/z=251 [M−H]⁻.

### E19. 6-(1-Hydroxy-1,3-dihydro-benzo[c][1,2]oxaborol-6-yloxy)-pyridine-2-carboxylic acid

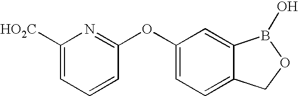

### 6-(1-Hydroxy-1,3-dihydro-benzo[c][1,2]oxaborol-6-yloxy)-pyridine-2-carboxylic acid

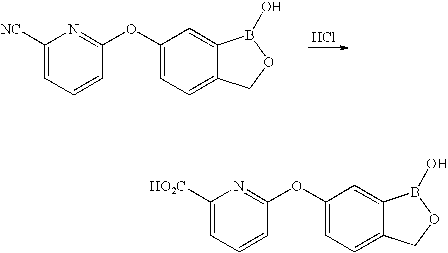

A mixture of 6-(1-hydroxy-1,3-dihydro-benzo[c][1,2]oxaborol-6-yloxy)-pyridine-2-carbonitrile (0.40 g, 1.59 mmol) in 6 N hydrochloric acid (5 mL) was stirred at reflux for 12 h. The mixture was concentrated to give the crude product which was purified by prep-HPLC (C18-SiO₂, acetonitrile/water/TFA) to give 0.192 g of product which is 91.84% pure. This material was purified by recrystallization from acetonitrile/water to give 58 mg of pure product as a white solid. Mp 279-281° C. ¹HNMR (400 MHz, DMSO-d₆) δ 8.02 (m, 1H), 7.80 (d, J=7.03 Hz, 1H), 7.48 (d, J=8.20 Hz, 1H), 7.44 (d, J=2.05 Hz, 1H), 7.29 (dd, J=8.20, 2.34 Hz, 1H), 7.24 (d, J=8.20 Hz, 1H), 5.02 (s, 2H). MS (ESI) m/z=272 [M+H]⁺.

### E20. 6-(1-Hydroxy-1,3-dihydro-benzo[c][1,2]oxaborol-6-yloxy)-nicotinic acid ethyl ester

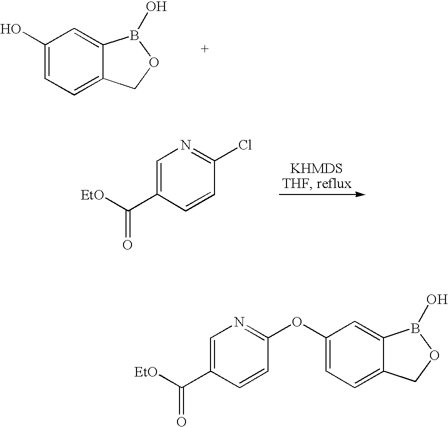

To a solution of 3H-benzo[c][1,2]oxaborole-1,6-diol (1.2 g, 8.0 mmol) in anhydrous dioxane (100 mL) was slowly added KHMDS (48 mL, 0.5 M solution in toluene, 24.0 mmol) at 0° C. After stirring for 15 min at room temperature, 6-chloro-nicotinic acid ethyl ester (2.97 g, 16.0 mmol) was added slowly to the reaction mixture at 0° C. The resulting mixture was stirred at 80° C. for 22 h. The reaction quenched by adding cold brine at 0° C. and the mixture was acidified to pH 3 using diluted hydrochloric acid. The resulting mixture was extract with EtOAc. The extract was washed with brine, dried and concentrated to dryness. The residue was purified by chromatography on silica gel (DCM/methanol=40:1) to give 0.521 g of material. This material was purified by prep-TLC (silica gel, THF/hexanes/AcOH=2:4:trace) to give 0.261 g of purer material which was purified again by chromatography on silica gel (DCM/methanol=40:1) to give 0.109 g of pure product as a pale-yellow solid. Mp 84-85° C. ¹H NMR (400 MHz, DMSO-d₆) δ 9.23 (s, 1H), 8.68 (d, J=2.34 Hz, 1H), 8.31 (dd, J=8.50, 2.34 Hz, 1H), 7.41-7.54 (m, 2H), 7.30 (dd, J=8.20, 2.34 Hz, 1H), 7.15 (d, J=8.50 Hz, 1H), 5.02 (s, 2H), 4.32 (q, J=7.03 Hz, 2H), 1.31 (t, J=7.03 Hz, 3H). MS (ESI) m/z=300 [M+H]⁺.

### E21. 6-(1-Hydroxy-1,3-dihydro-benzo[c][1,2]oxaborol-6-yloxy)-nicotinonitrile

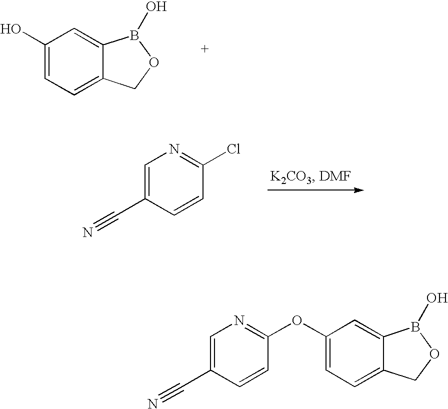

To a solution of 3H-benzo[c][1,2]oxaborole-1,6-diol (0.47 g, 3.13 mmol) in anhydrous DMF (15 mL) were added K₂CO₃ (1.30 g, 9.4 mmol) and 6-chloro-nicotinonitrile (0.868 g, 6.27 mmol) at room temperature. After stirring for 18 h at 85° C., the reaction mixture was cooled to room temperature. The solid was filtered out and dissolved into water (20 mL) and acidified to pH 3 using diluted hydrochloric acid. The precipitate was collected and washed with water and dried to give 0.612 g of crude product which was purified by recrystallization from EtOAc/hexanes to give 0.361 g of pure product as a white solid. Mp 156-157° C. ¹HNMR (400 MHz, DMSO-d₆) δ 9.25 (s, 1H), 8.64 (d, J=2.05 Hz, 1H), 8.33 (dd, J=8.50, 2.34 Hz, 1H), 7.40-7.53 (m, 2H), 7.19-7.32 (m, 2H), 5.02 (s, 2H). MS (ESI) m/z=253 [M+H]⁺.

### E22. 6-(1-Hydroxy-1,3-dihydro-benzo[c][1,2]oxaborol-6-yloxy)-nicotinamide

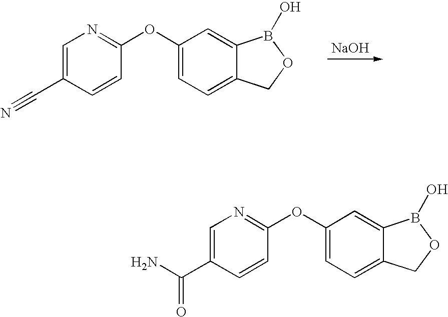

To a solution of 6-(1-hydroxy-1,3-dihydro-benzo[c][1,2]oxaborol-6-yloxy)-nicotinonitrile (0.79 g, 3.13 mmol) in MeOH (10 mL)/dioxane (10 mL) was added aqueous NaOH (1.25 g in 5 mL of water). After stirring at 60° C. for 3 h, the reaction mixture was cooled to 0° C. and acidified to pH 3 using diluted hydrochloric acid. The precipitate was collected and washed with water and dried to give the crude product which was purified by chromatography on silica gel (DCM/methanol=40:1) to give 0.123 g of product. This material was purified again by recrystallization from EtOAc/hexanes to give 0.048 g of pure product as a white solid. Mp 196-198° C. ¹HNMR (400 MHz, DMSO-d₆) δ 9.20 (s, 1H), 8.60 (d, J=2.34 Hz, 1H), 8.27 (dd, J=8.50, 2.34 Hz, 1H), 8.02 (s, 1H), 7.43-7.53 (m, 3H), 7.28 (dd, J=8.20, 2.34 Hz, 1H) 7.11 (d, J=8.50 Hz, 1H), 5.03 (s, 2H). MS (ESI) m/z=271 [M+H]⁺.

### E23. 6-(6-Aminomethyl-pyridin-2-yloxy)-3H-benzo[c][1,2]oxaborol-1-ol

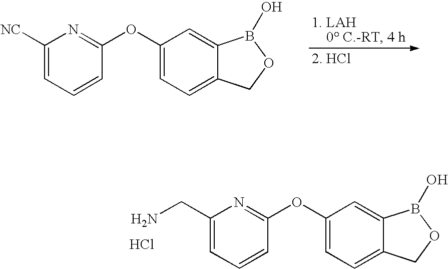

To a solution of 6-(1-hydroxy-1,3-dihydro-benzo[c][1,2]oxaborol-6-yloxy)-pyridine-2-carbonitrile (0.4 g, 1.59 mmol) in anhydrous THF (20 mL) was slowly added LiAlH₄ (0.151 g, 3.97 mmol) at 0° C. under nitrogen. The resulting mixture was stirred at 0° C. to room temperature for 4 h. The reaction was quenched by adding water at −20° C. and the mixture was acidified to pH 2 using diluted hydrochloric acid. The mixture was extracted with 33% ethanol in EtOAc. The extract was washed with brine and dried to give the crude product which was purified by chromatography on silica gel (EtOAc/MeOH/conc. NH₃—H₂O=5:5:1) to give the product. This material was stirred with water and acidified to pH 2 using diluted hydrochloric acid and lyophilized to afford 0.202 g of product which was stirred with 50 mL of hot EtOAc and the un-dissolved solid was collected and washed with EtOAc to give 0.146 g of pure product as a white powder. Mp 182-183° C. ¹H NMR (400 MHz, DMSO-d₆) δ 9.24 (br. s, 1H), 8.34 (br. s., 3H), 7.91 (t, J=7.77 Hz, 1H), 7.41-7.58 (m, 2H), 7.18-7.34 (m, 3H), 6.90 (d, J=8.21 Hz, 1H), 5.01 (s, 2H), 4.08 (q, J=5.76 Hz, 2H). MS (ESI) m/z=257 [M+H]⁺.

### E24. 2-(1-Hydroxy-1,3-dihydro-benzo[c][1,2]oxaborol-6-yloxy)-pyrimidine-5-carboxylic acid methyl ester

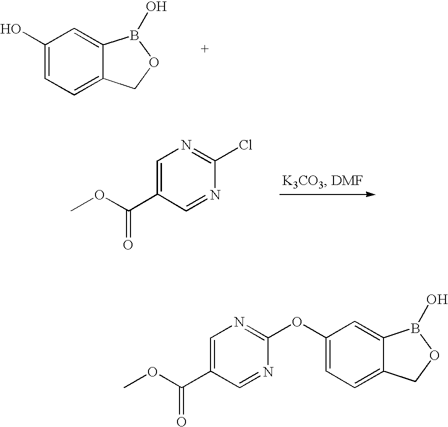

To a solution of 3H-benzo[c][1,2]oxaborole-1,6-diol (0.5 g, 3.33 mmol) in anhydrous DMF (15 mL) were added K₂CO₃ (1.382 g, 10.0 mmol) and 2-chloro-pyrimidine-5-carboxylic acid methyl ester (0.575 g, 3.33 mmol) at room temperature. After stirring at room temperature for 25 h, the reaction mixture was cooled to 0° C. diluted with water (20 mL) and acidified to pH 2 using diluted hydrochloric acid. The white precipitate was collected, washed with water and dried to give 0.678 g of pure product. Mp 117-118° C. ¹HNMR (400 MHz, DMSO-d₆) δ 9.26 (s, 1H), 9.08 (s, 2H), 7.44-7.57 (m, 2H), 7.31-7.40 (m, 1H), 5.03 (s, 2H), 3.88 (s, 3H). MS (ESI) m/z=287 [M+H]⁺.

### E25. 2-(1-Hydroxy-1,3-dihydro-benzo[c][1,2]oxaborol-6-yloxy)-pyrimidine-5-carboxylic acid

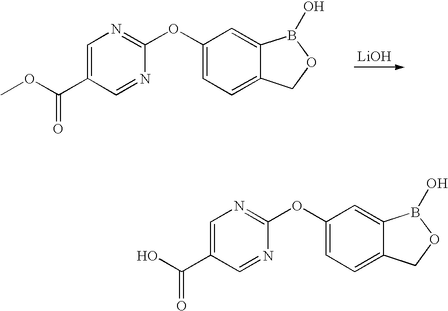

To a solution of 2-(1-hydroxy-1,3-dihydro-benzo[c][1,2]oxaborol-6-yloxy)-pyrimidine-5-carboxylic acid methyl ester (0.5 g, 1.75 mmol) in methanol (20 mL) was added aqueous LiOH (0.419 g in 15 mL of water, 17.5 mmol) at 0° C. The resulting mixture was stirred at room temperature for 1.5 h. After removed most of the methanol, the reaction mixture was cooled to 0° C. and acidified to pH 2 using diluted hydrochloric acid. The white precipitate was collected, washed with water and dried to give the crude product which was purified by chromatography on silica gel (hexane/THF/AcOH=2:1:trace) to give 0.102 g of product which is 92% pure by HPLC. This material was again purified by prep-HPLC to give 39 mg of pure product. Mp 195-196° C. ¹HNMR (400 MHz, DMSO-d₆) δ 9.27 (br. s., 1H), 9.04 (s, 2H), 7.51-7.49 (m, 2H), 7.35 (dd, J=8.4 Hz, 2.0 Hz, 1H), 5.03 (s, 2H). MS (ESI) m/z=273 [M+H]⁺.

### E26. 5-(1-Hydroxy-1,3-dihydro-benzo[c][1,2]oxaborol-6-yloxy)-pyrazine-2-carboxylic acid

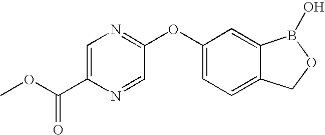

### 5-(1-Hydroxy-1,3-dihydro-benzo[c][1,2]oxaborol-6-yloxy)-pyrazine-2-carboxylic acid methyl ester

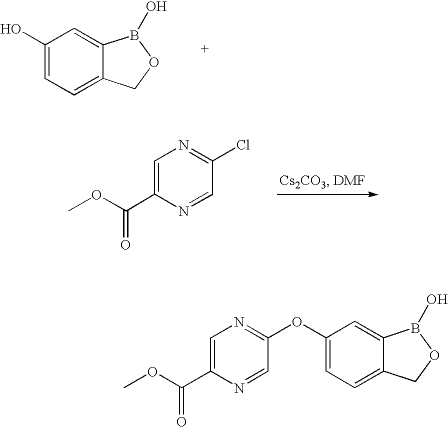

To a solution of 3H-benzo[c][1,2]oxaborole-1,6-diol (0.37 g, 2.47 mmol) in anhydrous DMF (8 mL) were added Cs₂CO₃ (2.01 g, 2.71 mmol) and 5-chloro-pyrazine-2-carboxylic acid methyl ester (0.468 g, 2.71 mmol) at room temperature. After stirring at 90° C. for 1.5 h, the reaction mixture was cooled to 0° C., diluted with water (10 mL) and acidified to pH 3 using diluted hydrochloric acid. The off-white precipitate was collected, washed with water and dried to give the crude product which was purified by chromatography on silica gel (DCM/MeOH=40:3) to give 0.470 g (66.5% yield) of product. MS (ESI) m/z=287 [M+H]⁺.

### E27. 5-(1-Hydroxy-1,3-dihydro-benzo[c][1,2]oxaborol-6-yloxy)-pyrazine-2-carboxylic acid

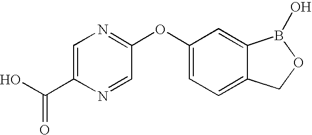

To a solution of 5-(1-hydroxy-1,3-dihydro-benzo[c][1,2]oxaborol-6-yloxy)-pyrazine-2-carboxylic acid methyl ester (0.47 g, 1.64 mmol) in methanol (16 mL) was added aqueous LiOH-H₂O (0.345 g in 12 mL of water, 8.21 mmol) at 0° C. The resulting mixture was stirred at 0° C. for 1 h. The reaction mixture was acidified to pH 2 using diluted hydrochloric acid. The white precipitate was collected, washed with water and 30% of EtOAc/hexanes and dried to give 0.392 g (87.9% yield) of pure product. Mp 202-204° C. ¹HNMR (400 MHz, DMSO-d₆) δ 9.28 (s, 1H), 8.74 (d, J=1.2 Hz, 1H), 8.66 (d, J=1.2 Hz, 1H), 7.53-7.50 (m, 2H), 7.37 (dd, J=8.4 Hz, 2.0 Hz, 1H), 5.03 (s, 2H). MS (ESI) m/z=271 [M−H]⁻.

### E28. 2-(1-Hydroxy-1,3-dihydro-benzo[c][1,2]oxaborol-6-yloxy)-thiazole-4-carboxylic acid methyl ester

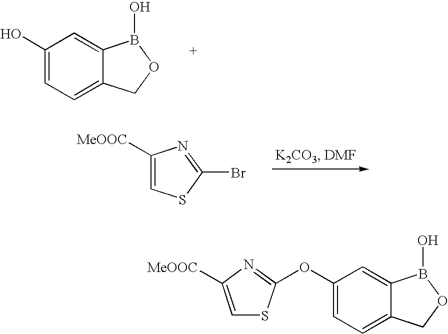

To a solution of 3H-benzo[c][1,2]oxaborole-1,6-diol (0.5 g, 3.33 mmol) in anhydrous DMF (15 mL) was added potassium carbonate (1.38 g, 9.99 mmol) followed by the addition of 2-bromo-thiazole-4-carboxylic acid methyl ester (0.74 g, 3.33 mmol). The resulting mixture was heated at 80° C. for 24 h. The reaction mixture was cooled and extracted with EtOAc, washed with water, brine, dried over Na₂SO₄, filtered, and concentrated to give crude product which was purified by prep HPLC using CH₃CN/H₂O (0.1% AcOH) as the eluent to yield 2-(1-hydroxy-1,3-dihydro-benzo[c][1,2]oxaborol-6-yloxy)-thiazole-4-carboxylic acid methyl ester (0.01 g) as a white solid after lyophilization. Mp 109.2-111.5° C. ¹H NMR (400 MHz, DMSO-d₆) δ 9.34 (s, 1H), 8.08 (s, 1H), 7.62 (d, J=1.6 Hz, 1H), 7.56 (d, J=8.4 Hz, 1H), 7.47 (dd, J=8.4, 2.6 Hz, 1H), 5.03 (s, 2H), 3.78 (s, 3H). MS (ESI) m/z=292 [M+H]⁺.

### E29 6-([1,3,4]Thiadiazol-2-yloxy)-3H-benzo[c][1,2]oxaborol-1-ol

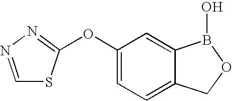

### Step 1. 2-Hydroxy-4-([1,3,4]thiadiazol-2-yloxy)-benzoic acid methyl ester

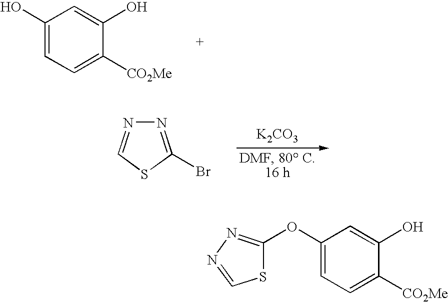

A solution of 2,4-dihydroxy-benzoic acid methyl ester (1.0 g, 6 mmol), 5-bromothiadiazole (1.0 g, 6 mmol) and K₂CO₃ (1.25 g, 9 mmol) in DMF (15 mL) was heated at 80° C. for 16 hours. Water (25 mL) was added and the mixture extracted with EtOAc (2×10 mL). The organic extracts were washed with water (10 mL), dried and concentrated in vacuo. The residue was purified by silica gel flash column chromatography (hexane:EtOAc 80:20) to give 2-hydroxy-4-([1,3,4]thiadiazol-2-yloxy)-benzoic acid methyl ester (0.23 g, 16%). ¹H NMR (400 MHz, CDCl₃): δ 10.98 (s, 1H), 8.82 (s, 1H), 7.91 (d, J=8.8 Hz, 1H), 6.89 (dd, J=2.4, 12 Hz, 2H), 3.96 (s, 3H). MS (ESI) m/z=253 [M+H]⁺.

### Step 2. 4-([1,3,4]Thiadiazol-2-yloxy)-2-trifluoromethanesulfonyloxy-benzoic acid methyl ester

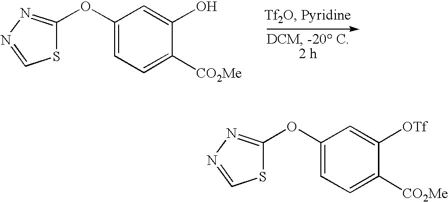

To a solution of 2-hydroxy-4-([1,3,4]thiadiazol-2-yloxy)-benzoic acid methyl ester (0.41 g, 1.62 mmol) in DCM (20 mL) at −20° C. was added pyridine (0.65 mL, 8.13 mmol) followed by Tf₂O (0.41 mL, 2.43 mmol). The resulting solution was allowed to warm to room temperature over 2 hours then quenched by the addition of ice-water (10 mL) and extracted with DCM (2×10 mL). The organic extracts were washed with 2N HCl (5 mL), dried and concentrated in vacuo. The residue was purified by silica gel flash column chromatography (hexane:EtOAc 70:30) to give 4-([1,3,4]thiadiazol-2-yloxy)-2-trifluoromethanesulfonyloxy-benzoic acid methyl ester (0.45 g, 97%). ¹H NMR (400 MHz, CDCl₃): δ 8.79 (s, 1H), 8.12 (d, J=9.2 Hz, 1H), 7.51 (dd, J=2.4, 8.4 Hz, 1H), 7.38 (d, J=1.6 Hz, 1H), 3.90 (s, 3H). MS (ESI) m/z=385 [M+H]⁺.

### Step 3. 2-(4,4,5,5-Tetramethyl-[1,3,2]dioxaborolan-2-yl)-4-([1,3,4]thiadiazol-2-yloxy)-benzoic acid methyl ester

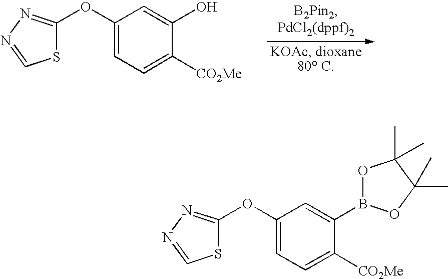

A solution of 4-([1,3,4]thiadiazol-2-yloxy)-2-trifluoromethanesulfonyloxy-benzoic acid methyl ester (0.36 g, 1.19 mmol) in dioxane (15 mL) was degassed for 15 minutes with bubbling N₂. Bispinacolatodiboron (0.49 g, 1.42 mmol), PdCl₂(dppf)₂ (0.087 g, 0.11 mmol) and KOAc (0.35 g, 3.59 mmol) were added and the solution stirred at 80° C. for 20 hours. After cooling to room temperature the mixture was filtered through a pad of celite and concentrated in vacuo. The residue was dissolved in EtOAc (20 mL), washed with water (2×10 mL), dried and concentrated in vacuo. The residue was purified by silica gel flash column chromatography (hexane:EtOAc 70:30) to give 2-(4,4,5,5-tetramethyl-[1,3,2]dioxaborolan-2-yl)-4-([1,3,4]thiadiazol-2-yloxy)-benzoic acid methyl ester (0.38 g, 3:1 mixture with SM). ¹H NMR (400 MHz, CDCl₃): δ 8.76 (s, 1H), 7.99 (d, J=6.4 Hz, 1H), 7.42-7.38 (m, 2H), 3.89 (s, 3H), 1.38 (s, 12H). MS (ESI) m/z=363 [M+H]⁺.

### Step 4. 6-([1,3,4]Thiadiazol-2-yloxy)-3H-benzo[c][1,2]oxaborol-1-ol

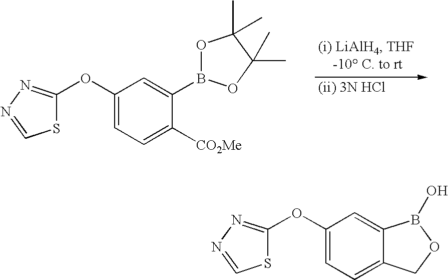

To a solution of 2-(4,4,5,5-tetramethyl-[1,3,2]dioxaborolan-2-yl)-4-([1,3,4]thiadiazol-2-yloxy)-benzoic acid methyl ester (0.26 g, 0.72 mmol) in THF (15 mL) at −10° C. was added LiAlH₄ (0.04 g, 1.07 mmol). The reaction mixture was allowed to warm to room temperature over 6 hours then quenched by the addition of 3N HCl (3 mL) at 0° C. The mixture was stirred for 2 hours, extracted with EtOAc (2×10 mL), washed with water (10 mL), dried and concentrated in vacuo. The residue was purified by silica gel flash column chromatography (DCM; MeOH 95:5) to give 6-([1,3,4]thiadiazol-2-yloxy)-3H-benzo[c][1,2]oxaborol-1-ol (0.05 g, 35%). ¹H NMR (400 MHz, DMSO): δ 9.15 (s, 1H), 7.65 (d, J=1.6 Hz, 1H), 7.54-7.50 (m, 2H), 5.00 (s, 2H). MS (ESI) m/z=235 [M+H]⁺.

### E30. 6-Cyclopentyloxy-3H -benzo[c][1,2]oxaborol-1-ol

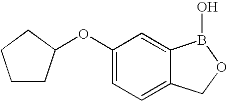

### Step 1. 4-Cycloyentyloxy-2-hydroxy-benzaldehyde

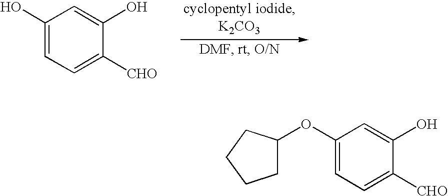

To a solution of 2,4-dihydroxy-benzaldehyde (5.0 g, 36.0 mmol) in DMF (60 mL), potassium carbonate (7.46 g, 54.0 mmol) was added at 0° C., followed by addition of cyclopentyl iodide(6.35 g, 32.4mmol). The resulting mixture was stirred at rt overnight. The reaction mixture was extracted with EtOAc and washed with water, brine, dried over Na₂SO₄, and concentrated under reduced pressure. The crude product was dissolved in 200 mL of hexane and stirred at rt for 30 min. Solid (unreacted starting material) precipitated out was filtered, hexane layer was concentrated and purified by Biotage (20% DCM/Hexane) to get 4-cyclopentyloxy-2-hydroxy-benzaldehyde as a colorless oil (1.74 g, 23% yield). ¹HNMR (400 MHz, CDCl₃) δ 11.49 (s, 1H), 9.69 (s, 1H), 7.40 (d, J=8.8 Hz, 1H), 6.50 (dd, J=8.8, 2.4 Hz, 1H), 6.39 (d, J=2.0 Hz, 1H), 4.82 (m, 1H), 1.99-1.79 (m, 6H), 1.78-1.59 (m, 2H). (ESI) m/z=205 [M−H]⁻.

### Step 2. 5-(Cyclopentyloxy)-2-formylphenyl trifluoromethanesulfonate

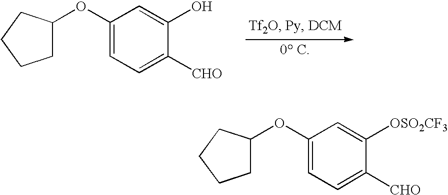

To a solution of 4-cyclopentyloxy-2-hydroxy-benzaldehyde (1.74 g, 8.46 mmol) and pyridine (3.3 g, 42.0 mmol) in dichloromethane (20 mL) was slowly added Tf₂O (3.57 g, 12.66 mol) at −10 to 0° C. over a period of 30 min. The reaction mixture was stirred at 0° C. for 30 min. Ice-water was added, and the mixture was acidified with 6M hydrochloric acid to pH 2. The resulting mixture was extracted with 50% EtOAc/hexanes (2×75 mL), washed with brine, dried over Na₂SO₄, and concentrated to give (1.8 g, 64% yield) as a pale-yellow oil. ¹HNMR (400 MHz, CDCl₃) δ 10.11 (s, 1H), 7.90 (d, J=9.2 Hz, 1H), 6.98 (dd, J=9.2, 2.4 Hz, 1H), 6.82 (d, J=2.4 Hz, 1H), 4.85 (m, 1H), 2.00-1.80 (m, 6H), 1.78-1.65 (m, 2H).

### Step 3. 4-Cyclopentyloxy-2-(4,4,5,5-tetramethyl-[1,3,2]dioxaborolan-2-yl)-benzaldehyde

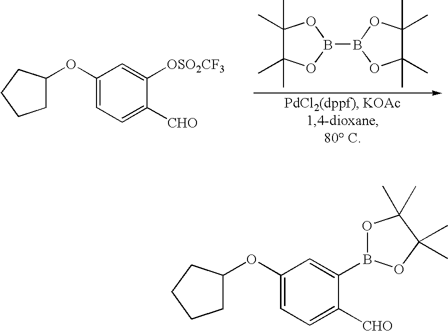

5-(Cyclopentyloxy)-2-formylphenyl trifluoromethanesulfonate (1.1 g, 3.25 mmol) in 1,4-dioxane (20 mL) was degassed for 30 min under nitrogen gas. Bis(pinacolato)diboron (1.65 g, 6.50 mmol), potassium acetate (1.3 g, 13.0 mmol), and [1,1′-bis(diphenylphosphino)ferrocene]palladium(II)chloride (0.24 g, 0.033 mmol) were added. The reaction mixture was heated at 80° C. for 1 h, extracted with EtOAc and washed with water, brine, dried over Na₂SO₄, and concentrated under reduced pressure to give crude product, which was purified by Biotage (1-25% EtOAc in hexane) to afford 4-cyclopentyloxy-2-(4,4,5,5-tetramethyl-[1,3,2]dioxaborolan-2-yl)-benzaldehyde (0.9 g, 88% yield) as a white semi solid. ¹H NMR (400 MHz, CDCl₃) δ 10.30 (s, 1H), 7.90 (d, J=8.8 Hz, 1H), 7.25 (d, J=2.4 Hz, 1H), 6.99 (dd, J=9.2, 2.8 Hz, 1H), 4.88 (m, 1H), 2.00-1.80 (m, 6H), 1.75-1.60 (m, 2H), 1.39 (s, 12H). MS (ESI) m/z=317 [M+H]⁺.

### Step 4. 6-Cyclopentyloxy-3H -benzo[c][1,2]oxaborol-1-ol

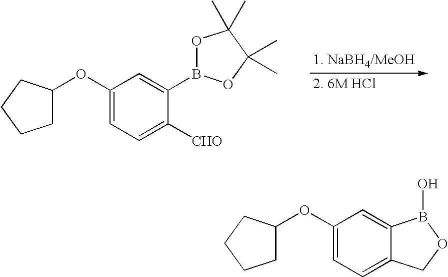

To a solution of 4-cyclopentyloxy-2-(4,4,5,5-tetramethyl-[1,3,2]dioxaborolan-2-yl)-benzaldehyde (0.5 g, 1.58 mmol) in methanol (20 mL) was slowly added NaBH₄ powder (0.18 g, 4.75 mmol) at 0-10° C. After stirred at room temperature for 1 h, the mixture was concentrated to remove one-third of methanol. The resulting mixture was cooled to 0° C., acidified to pH 3 using 6M hydrochloric acid. The reaction mixture was extracted with EtOAc and washed with water, brine, dried over Na₂SO₄, and concentrated under reduced pressure to give crude product, which was purified by Biotage (50% EtOAc in hexane) to afford 6-cyclopentyloxy-3H -benzo[c][1,2]oxaborol-1-ol (0.11 g, 32% yield) as a red sticky solid. ¹H NMR (400 MHz, DMSO-d₆) δ 9.10 (s, 1H), 7.28 (d, J=8.4 Hz, 1H), 7.21(s, 1H), 7.00 (d, J=7.2 Hz, 1H), 4.90 (s, 2H), 4.79 (m, 1H), 1.97-1.85 (m, 2H), 1.78-1.65 (m, 4H), 1.62-1.55 (m, 2H). MS (ESI) m/z=217 [M−H]⁻.

### E31. 4-(1-Hydroxy-1,3-dihydro-benzo[c][1,2]oxaborol-6-yloxy)-piperidine-1-carboxylic acid tert-butyl ester

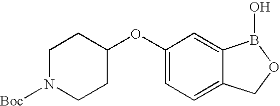

### Step 1. 4-(4-Formyl-3-hydroxy-phenoxy)-piperidine-1-carboxylic acid tert-butyl ester

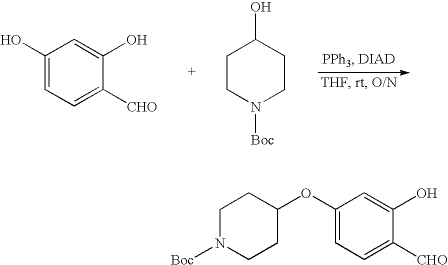

To a solution of 2,4-dihydroxy-benzaldehyde (10.0 g, 72.0 mmol) in THF (70 mL) was added triphenylphosphine (22.66 g, 86.4 mmol) and cooled to 0° C., 4-hydroxy-piperidine-1-carboxylic acid tert-butyl ester (16.0 g, 79.7 mmol) was added dropwise, followed by the addition of diisopropyl azo-dicarboxylate (17.47 g, 86.4 mmol). The resulting mixture was stirred at rt overnight. The reaction mixture was extracted with EtOAc and washed with water, brine, dried over Na₂SO₄, and concentrated under reduced pressure. The crude product was dissolved in 10% EtOAc/Hexane (200 mL) and stirred at rt for 30 min. Solid (unreacted starting material and triphenylphosphineoxide) precipitated out was filtered, and the organic layer was concentrated and purified by Biotage (20% EtOAc/Hexane) to 4-(4-formyl-3-hydroxy-phenoxy)-piperidine-1-carboxylic acid tert-butyl ester (7.5 g, mixture of product and 2,4-dihydroxy-benzaldehyde, 2:1). ¹H NMR (400 MHz, CDCl₃) δ 11.46 (s, 1H), 9.70 (s, 1H), 7.44 (d, J=8.8 Hz, 1H), 6.52 (dd, J=8.8, 2.0 Hz, 1H), 6.42 (d, J=2.0 Hz, 1H), 4.55 (m, 1H), 3.69-3.64 (m, 2H), 3.40-3.30 (m, 2H), 2.04-1.94 (m, 2H), 1.80-1.72 (m, 2H), 1.47 (s, 9H). MS (ESI) m/z=320 [M−H]⁻.

### Step 2. 4-(4-Formyl-3-trifluoromethanesulfonyloxy-phenoxy)-piperidine-1-carboxylic acid tert-butyl ester

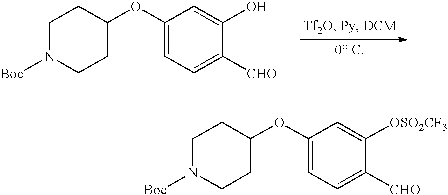

To a solution of 4-(4-formyl-3-hydroxy-phenoxy)-piperidine-1-carboxylic acid tert-butyl ester (7.0 g, 22.0 mmol) and pyridine (8.7 g, 110 mmol) in dichloromethane (100 mL) was slowly added Tf₂O (12.30 g, 44.0 mol) at −10 to 0° C. over a period of 45 min. The reaction mixture was stirred at 0° C. for 1.5 h. Ice-water was added, extracted with EtOAc, washed with saturated sodium bisulfite, and cold brine, dried over Na₂SO₄, and concentrated, purified by column chromatography (20-40% EtOAc/Hexane) to get 4-(4-formyl-3-trifluoromethanesulfonyloxy-phenoxy)-piperidine-1-carboxylic acid tert-butyl ester (7.5 g, 75% yield) as a pale-yellow oil. ¹H NMR (400 MHz, CDCl₃) δ 10.12 (s, 1H), 7.95 (d, J=8.8 Hz, 1H), 7.00 (dd, J=8.8, 2.4 Hz, 1H), 6.87 (d, J=2.0 Hz, 1H), 4.60 (m, 1H), 3.72-3.66 (m, 2H), 3.43-3.36 (m, 2H), 2.03-1.94 (m, 2H), 1.83-1.75 (m, 2H), 1.47 (s, 9H).

### Step 3. 4-[4-Formyl-3-(4,4,5,5-tetramethyl-[1,3,2]dioxaborolan-2-yl)-phenoxy]-piperidine-1-carboxylic acid tert-butyl ester

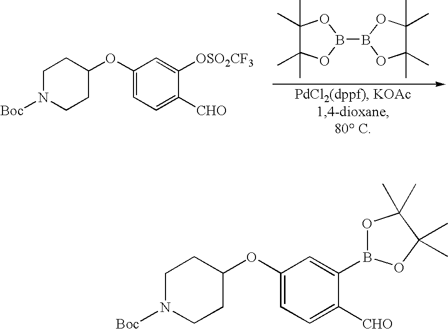

4-(4-Formyl-3-trifluoromethanesulfonyloxy-phenoxy)-piperidine-1-carboxylic acid tert-butyl ester (7.0 g, 15.0 mmol) in 1,4-dioxane (80 mL) was degassed for 30 min under nitrogen gas. Bis(pinacolato)diboron (4.7 g, 18.5 mmol), potassium acetate (4.4 g, 45.0 mmol), and [1,1′-bis(diphenylphosphino)ferrocene]palladium(II)chloride (0.55 g, 0.075 mmol) were added. The reaction mixture was heated at 80° C. for 40 min, extracted with EtOAc and washed with water, brine, dried over Na₂SO₄, and concentrated under reduced pressure to give crude product, which was purified by column chromatography (10-20% EtOAc in hexane) to afford the 4-[4-formyl-3-(4,4,5,5-tetramethyl-[1,3,2]dioxaborolan-2-yl)-phenoxy]-piperidine-1-carboxylic acid tert-butyl ester (4.5 g, 70% yield) as a white solid. ¹H NMR (400 MHz, CDCl₃) δ 10.36 (s, 1H), 7.93 (d, J=8.8 Hz, 1H), 7.30 (d, J=2.4 Hz, 1H), 7.01 (dd, J=8.8, 2.8 Hz, 1H), 4.60 (m, 1H), 3.70-3.62 (m, 2H), 3.45-3.35 (m, 2H), 2.00-1.90 (m, 2H), 1.82-1.75 (m, 2H), 1.47 (s, 9H), 1.40 (s, 12H).

### Step 4. 4-(1-Hydroxy-1,3-dihydro-benzo[c][1,2]oxaborol-6-yloxy)-piperidine-1-carboxylic acid tert-butyl ester

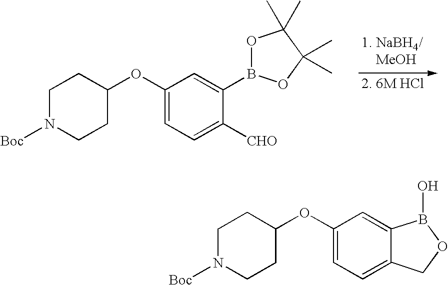

To a solution of 4-[4-formyl-3-(4,4,5,5-tetramethyl-[1,3,2]dioxaborolan-2-yl)-phenoxy]-piperidine-1-carboxylic acid tert-butyl ester (1.5 g, 3.5 mmol) in methanol (60 mL) was slowly added NaBH₄ powder (0.19 g, 5.22 mmol) at 0-10° C. After stirred at room temperature for 2.5 h, the mixture was concentrated to remove one-third of methanol. The resulting mixture was cooled to 0° C., acidified to pH 1 using 6M hydrochloric acid. The reaction mixture was stirred at rt, extracted with EtOAc and washed with water, brine, dried over Na₂SO₄, and concentrated under reduced pressure to give crude product, which was purified by Biotage (50% EtOAc in hexane) to afford 4-(1-hydroxy-1,3-dihydro-benzo[c][1,2]oxaborol-6-yloxy)-piperidine-1-carboxylic acid tert-butyl ester (0.54 g, 51% yield) as a white solid. Mp 110-111° C. ¹H NMR 400 MHz (DMSO-d₆) δ 9.10 (s, 1H), 7.30 (d, J=8.0 Hz, 1H), 7.27 (d, J=2.0 Hz, 1H), 7.09 (dd, J=8.0, 2.0 Hz, 1H), 4.90 (s, 2H), 4.55 (m, 1H), 3.70-3.60 (m, 2H), 3.20-3.15 (m, 2H), 1.98-1.85 (m, 2H), 1.60-1.51 (m, 2H), 1.40 (s, 9H). MS (ESI) m/z=334 [M−H]⁻.

### E32. 6-(Piperidin-4-yloxy)-3H-benzo[c][1,2]oxaborol-1-ol

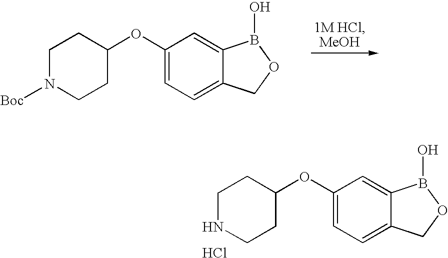

To a solution of 4-(1-hydroxy-1,3-dihydro-benzo[c][1,2]oxaborol-6-yloxy)-piperidine-1-carboxylic acid tert-butyl ester (0.54 g, 1.62 mmol) in methanol (7 mL) was added 1M HCl in ether (5.4 mL, 5.41 mmol). The reaction mixture was stirred at room temperature for 5 h, and concentrated to get 6-(piperidin-4-yloxy)-3H-benzo[c][1,2]oxaborol-1-ol hydrochloric salt (0.4 g, 92% yield) as a white solid. Mp 218-220° C. ¹H NMR (400 MHz, DMSO-d₆) δ 9.14 (s, 1H), 8.78 (br s, 1H), 7.34 (d, J=8.4 Hz, 1H), 7.30 (d, J=2.0 Hz, 1H), 7.12 (dd, J=8.4, 2.4 Hz, 1H), 4.90 (s, 2H), 4.64 (m, 1H), 3.33-3.22 (m, 2H), 3.16-3.08 (m, 2H), 2.12-2.07 (m, 2H), 1.88-1.80 (m, 2H). MS (ESI) m/z=324 [M−H]⁻.

### E33 (1,6-Dihydroxy-1,3-dihydro-benzo[c][1,2]oxaborol-3-yl)-acetic acid

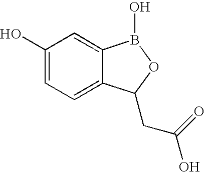

A solution of (1,6-dihydroxy-1,3-dihydro-benzo[c][1,2]oxaborol-3-yl)-acetic acid ethyl ester (0.20 g, 0.85 mmol) in methanol (7 mL) was treated with lithium hydroxide (0.10 g, 4.18 mmol) in water (7 mL) at 0° C. The solution was stirred at 0° C. for 2 hours then quenched with 2N HCl to pH 2. The mixture was diluted with brine and extracted with ethyl acetate. The organic extracts were dried (Na₂SO₄), filtered and concentrated to a light yellow solid. The residue was purified by silica gel flash column chromatography (AcOH:acetone:hexane; trace:1:2) and lyophilized to give (1,6-dihydroxy-1,3-dihydro-benzo[c][1,2]oxaborol-3-yl)-acetic acid as a white solid (0.06 g, 34%). ¹H NMR (400 MHz, DMSO-d6) δ 7.22 (d, J=8.20 Hz, 1H), 7.52 (d, J=2.34 Hz, 1H), 6.86 (dd, J=8.20, 1.95 Hz, 1H), 5.33 (dd, J=8.98, 3.90 Hz, 1H), 2.83 (dd, J=15.61, 3.90 Hz, 1H), 2.26 (dd, J=15.61, 8.98 Hz, 1H). MS (ESI) m/z: 207[M−1].

### E34 (1-Hydroxy-6-pyridin-3-yloxy)-1,3-dihydro-benzo[c][1,2]oxaborol-3-yl)-acetic acid

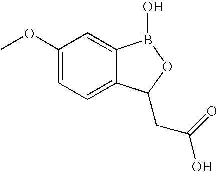

### Step 1: [1-Hydroxy-6methoxy-1,3-dihydro-benzo[c][1,2]oxaborol-3-yl]-acetic acid ethyl ester

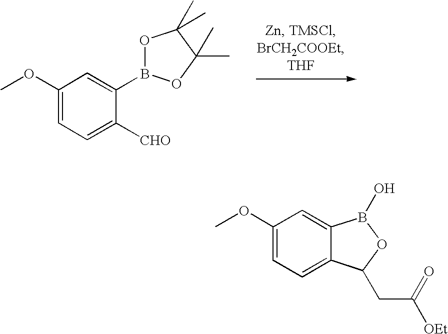

To a suspension of zinc dust (1.46 g, 22.5 mmol) in THF (10 mL) was added trimethylsilyl chloride (0.28 mL, 2.25 mmol) at 40° C. The mixture was heated to 55° C. and stirred for 15 minutes. After cooling down to 37° C., ethyl bromoacetate (2.16 mL, 19.5 mmol) was slowly added to the reaction mixture at 37-40° C. After addition, the resulting mixture was allowed to cool to room temperature over 30 minutes. This solution was added to a solution of 4-methoxy-2-(4,4,5,5)tetramethyl-[1,3,2]dioxaborolan-2-yl)benzaldehyde (1.15 g, 4 mmol) in THF (6 mL) at 0° C. The mixture was stirred for 10 minutes before treating with saturated NH₄Cl (10 mL) and extracted with EtOAc (2×25 mL). The organic extracts were washed with brine, dried and concentrated in vacuo. The residue was diluted with H₂O and lyophilized to give [1-hydroxy-6methoxy-1,3-dihydro-benzo[c][1,2]oxaborol-3-yl]-acetic acid ethyl ester (1.1 g, 100%). ¹H NMR (400 MHz, CD₃OD-d6) δ 7.25 (m, 1H), 7.10 (s, 1H), 7.00 (m, 1H), 5.50 (m, 1H), 4.19 (q, J=6.6 Hz, 2 H), 3.80 (s, 3H), 2.90 (m, 1H), 2.50 (m, 1H), 1.20 (t, J=6.6 Hz, 3H).

Step 2: (1-Hydroxy-6-methoxy)-1,3-dihydro-benzo[c][1,2]oxaborol-3-yl)-acetic acid

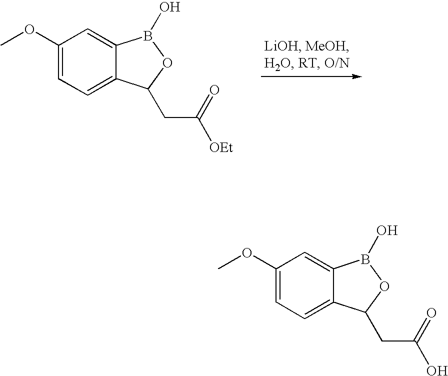

To a solution of [1-hydroxy-6methoxy-1,3-dihydro-benzo[c][1,2]oxaborol-3-yl]-acetic acid ethyl ester (0.320 g, 1.36 mmol) in MeOH (12 mL) and water (3 mL) was added LiOH (0.040 g) at 0° C. The resulting mixture was stirred at room temperature for 24 hours then cooled to 0° C. The reaction mixture was acidified to pH 3 using 6M HCl then concentrated in vacuo. The residue was purified by silica gel flash column chromatography to give (1-hydroxy-6-methoxy)-1,3-dihydro-benzo[c][1,2]oxaborol-3-yl)-acetic acid (0.200 g, 66%). mp 110-112° C. ¹H NMR (400 MHz, DMSO-dδ 12.38 (s, 1H), 9.18 (s, 1H), 7.36 (d, J=8.4 Hz, 1H), 7.20 (s, 1H), 7.05 (m, 1H), 5.38 (m, 1H), 3.76 (s, 3H), 2.90 (m, 1H), 2.28 (m, 1H). MS (ESI) m/z=221 [M−H]−. HPLC: 98.05% (220 nm); 98.79% (Maxplot).

### E35 (1-Hydroxy-6-phenoxy-1,3-dihydro-benzo[c][1,2]oxaborol-3-yl)-acetic acid ethyl ester

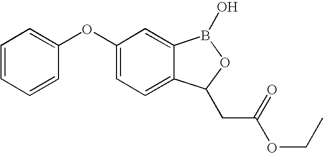

### Step 1. 2-Bromo-4-phenoxy-benzaldehyde

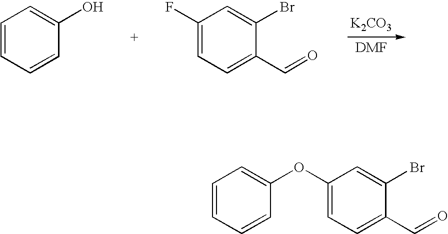

To a solution of phenol (6.732 g, 73.9 mmol) and 2-bromo-4-fluoro-benzaldehyde (15.0 g, 73.9 mmol) in anhydrous DMF (90 mL) was added K₂CO₃ (20.42 g, 148 mmol) at room temperature. The mixture was then stirred at 100° C. for 16.5 h. After filtration, the filtrate was concentrated to dryness. The residue was dissolved in EtOAc and washed with brine to pH 7, dried and concentrated to give the crude product which was recrystallized from EtOAc/hexanes to afford 16.821 g (82.1% yield) of pure product as a white crystal. ¹HNMR (400 MHz, CDCl₃) δ 10.25 (s, 1H), 7.90 (d, J=8.50 Hz, 1H), 7.44 (t, J=7.91 Hz, 2H), 7.20-7.33 (m, 1H), 7.17 (d, J=2.34 Hz, 1H), 7.09 (d, J=7.91 Hz, 2H) 6.98 (dd, J=8.64, 2.34 Hz, 1H).

### Step 2. 4-Phenoxy-2-(4,4,5,5-tetramethyl-[1,3,2]dioxaborolan-2-yl)-benzaldehyde

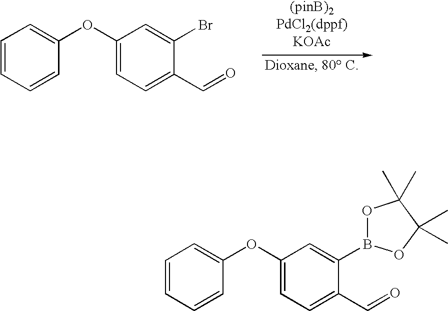

To a solution of 2-bromo-4-phenoxy-benzaldehyde (3.0 g, 10.8 mmol) and bis(pinacolato)diborane (3.573 g, 14.1 mmol) in dioxane (600 mL) was added KOAc (3.188 g, 32.5 mmol). After degassed with nitrogen for 15 min, PdCl₂(dppf) (0.792 g, 1.08 mol) was added to the reaction mixture. The mixture was stirred at 80° C. for 1.5 h. The reaction was quenched by adding ice-water (50 mL). The resulting mixture was extract with 50% EtOAc/hexanes (2×50 mL). The extract was washed with brine, dried and concentrated to dryness. The residue was purified by chromatography on silica gel (EtOAc/hexanes=3:40) to give 3.50 g (100% yield) of product as pale-yellow waxy solid. ¹HNMR (400 MHz, CDCl₃) δ 10.39 (s, 1H), 7.92 (d, J=8.50 Hz, 1H), 7.33-7.50 (m, 3H), 7.19 (t, J=7.47 Hz, 1H), 6.95-7.11 (m, 3H), 1.38 (s, 12H).

### Step 3. (1-Hydroxy-6-phenoxy-1,3-dihydro-benzo[c][1,2]oxaborol-3-yl)-acetic acid ethyl ester

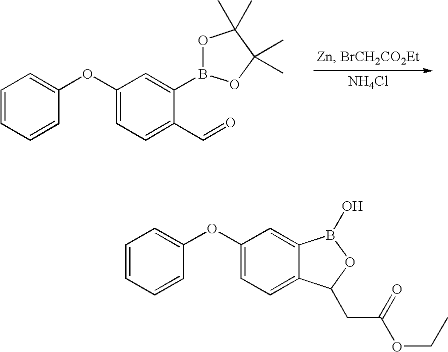

A mixture of 4-phenoxy-2-(4,4,5,5-tetramethyl-[1,3,2]dioxaborolan-2-yl)-benzaldehyde (2.0 g, 6.17 mmol), ethyl bromoacetate (3.091 g, 18.5 mmol), zinc dust (6.07 g) and NH₄Cl (2.43 g) was thoroughly grounded in a mortar and pestle. The resulting mixture was kept at room temperature (20° C.) for 3.5 h. The mixture was treated with saturated NH₄Cl (50 mL) and extracted with ether (3×50 mL). The extract was washed with brine, dried and concentrated to dryness. The residue was purified by chromatography on silica gel (EtOAc/hexanes=2:5) to give 1.351 g (70.1% yield) of product as a colorless oil. ¹HNMR (400 MHz, DMSO-d₆) δ 9.24 (s, 1H), 7.46 (d, J=8.20 Hz, 1H), 7.39 (t, J=7.76 Hz, 2H), 7.22 (d, J=2.05 Hz, 1H), 7.10-7.17 (m, 2H), 7.00 (d, J=8.20 Hz, 2H), 5.44 (dd, J=8.78, 3.81 Hz, 1H), 4.08 (q, J=7.22 Hz, 2H), 3.03 (dd, J=15.81, 3.81 Hz, 1H), 2.42 (dd, J=15.66, 8.93 Hz, 1H), 1.17 (t, J=7.22 Hz, 3H). MS (ESI) m/z=313 [M+H]⁺.

### E36 (1-Hydroxy-6-phenoxy-1,3-dihydro-benzo[c][1,2]oxaborol-3-yl)-acetic acid

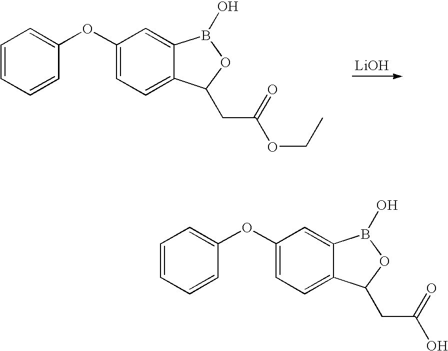

To a solution of (1-hydroxy-6-phenoxy-1,3-dihydro-benzo[c][1,2]oxaborol-3-yl)-acetic acid ethyl ester (0.3 g, 0.960 mmol) in methanol (9 mL) was added aqueous LiOH—H₂O (0.202 g in 7 mL of water, 4.80 mmol) at 0° C. The resulting mixture was stirred at 0° C. for 5 h. The reaction mixture was acidified to pH 2 using diluted hydrochloric acid. The mixture was extracted with EtOAc (2×20 mL). The extract was washed with brine and dried to give the crude product which was purified by chromatography on silica gel (acetone/hexanes=1:2) to give 0.201 g (73.3% yield) of pure product as white powder. Mp 132-134° C. ¹HNMR (400 MHz, DMSO-d6) δ 9.27 (s, 1H), 7.52 (d, J=8.0 Hz, 1H) 7.46-7.42 (m, 2H), 7.28 (d, J=2.4 Hz, 1H), 7.23-7.17 (m, 2H), 7.08-7.05 (m, 2H), 5.47 (dd, J=8.8, 4.0 Hz, 1H), 2.98 (dd, J=15.2, 4.0 Hz, 1H), 2.38 (dd, J=15.6, 9.2 Hz, 1H). MS (ESI) m/z=283 [M−H]⁻.

### E37 (3S) (1-hydroxy-6-phenoxy-1,3-dihydro-benzo[c][1,2]oxaborol-3-yl)-acetic acid

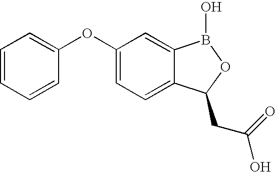

1.16 g of (1-Hydroxy-6-phenoxy-1,3-dihydro-benzo[c][1,2]oxaborol-3-yl)-acetic acid ethyl ester (E35) was separated by preparative HPLC using a CHIRALPAK® IC column (250×50 mm) using a mobile phase composition of 10% ethanol in hexane containing 0.1% of trifluoroacetic acid at a flow rate of 120 ml/min at ambient temperature. The sample size was 5 ml at a concentration of 23 g/l, giving a production rate of 2.76 g/hour. The second peak collected, (3S-(1-hydroxy-6-phenoxy-1,3-dihydro-benzo[c][1,2]oxaborol-3-yl)-acetic acid ethyl ester) had a purity of 99.6% ee.

To a solution of 3S-1-hydroxy-6-phenoxy-1,3-dihydro-benzo[c][1,2]oxaborol-3-yl)-acetic acid ethyl ester (0.491 g, 1.57 mmol, peak 2) in methanol (9 mL) was added a solution of LiOH (0.188 g, 7.86 mmol) in water (7 mL) at 0° C. The resulting mixture was stirred at 0° C. for 5 hours then acidified to pH=2 with dilute hydrochloric acid and extracted with EtOAc (2×20 mL). The organic extracts were washed with brine, dried over sodium sulfate and concentrated in vacuo. The residue was purified by silica gel flash column chromatography (acetone/hexanes=1:2) to give pure product as a white powder after lyophilization (0.320 g, 71.7%); mp 142-143° C. 1HNMR (400 MHz, DMSO-d) δ 9.26 (s, 1H), 7.52 (d, J=8.0 Hz, 1H) 7.46-7.42 (m, 2H), 7.28 (d, J=2.4 Hz, 1H), 7.23-7.17 (m, 2H), 7.08-7.04 (m, 2H), 5.48 (dd, J=8.8, 4.0 Hz, 1H), 2.98 (dd, J=15.2, 4.0 Hz, 1H), 2.37 (dd, J=15.6, 9.2 Hz, 1H). MS (ESI) m/z=283 [M−H]−.

### E38 (3R) (1-hydroxy-6-phenoxy-1,3-dihydro-benzo[c][1,2]oxaborol-3-yl)-acetic acid

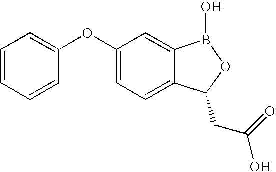

1.16 g of (1-Hydroxy-6-phenoxy-1,3-dihydro-benzo[c][1,2]oxaborol-3-yl)-acetic acid ethyl ester (E35) was separated by preparative HPLC using a CHIRALPAK® IC column (250×50 mm) using a mobile phase composition of 10% ethanol in hexane containing 0.1% of trifluoroacetic acid at a flow rate of 120 ml/min at ambient temperature. The sample size was 5 ml at a concentration of 23 g/l, giving a production rate of 2.76 g/hour. The 1st peak collected (3R-(1-hydroxy-6-phenoxy-1,3-dihydro-benzo[c][1,2]oxaborol-3-yl)-acetic acid ethyl ester) had a purity of 99.9% ee.

To a solution of 3R-(1-hydroxy-6-phenoxy-1,3-dihydro-benzo[c][1,2]oxaborol-3-yl)-acetic acid ethyl ester (0.4 g, 1.28 mmol, peak 1) in methanol (8 mL) was added a solution of LiOH (0.153 g, 6.40 mmol) in water (6 mL) at 0° C. The resulting mixture was stirred at 0° C. for 5 hours then acidified to pH=2 with dilute hydrochloric acid and extracted with EtOAc (2×20 mL). The organic extracts were washed with brine, dried over sodium sulfate and concentrated in vacuo. The residue was purified by silica gel flash column chromatography (acetone/hexanes=1:2) to give pure product as a white powder after lyophilization (0.370 g, 100%); mp 146-147° C. 1HNMR (400 MHz, DMSO-d) δ 9.27 (s, 1H), 7.52 (d, J=8.0 Hz, 1H) 7.46-7.42 (m, 2H), 7.28 (d, J=2.4 Hz, 1H), 7.23-7.17 (m, 2H), 7.08-7.05 (m, 2H), 5.47 (dd, J=8.8, 4.0 Hz, 1H), 2.98 (dd, J=15.2, 4.0 Hz, 1H), 2.38 (dd, J=15.6, 9.2 Hz, 1H). MS (ESI) m/z=283 [M−H]−.

### E39 3-(1-Hydroxy-6-phenoxy-1,3-dihydro-benzo[c][1,2]oxaborol-3-yl)-propionic acid

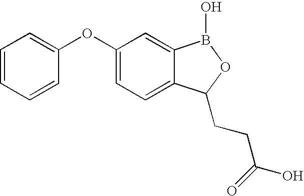

### Step 1 3-(tert-Butyl-dimethyl-silanyloxy)-propylmagnesium bromide

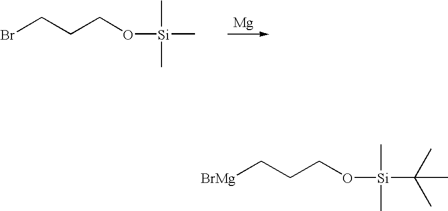

To a mixture of magnesium turnings (0.439 g, 18.04 mmol), iodine (catalytic amount) in anhydrous THF (10 mL) was slowly added (3-bromo-propoxy)-trimethyl-silane (3.52 g, 13.88 mmol) in THF (15 mL) at room temperature under nitrogen. After the reaction initiated, the speed of the addition of the (3-bromo-propoxy)-trimethyl-silane solution was controlled to maintain the temperature of the reaction mixture at 30-35° C. After the addition completed the resulting mixture was stirred at 40° C. for 1 h to afford a solution of 3-(tert-butyl-dimethyl-silanyloxy)-propylmagnesium bromide.

### Step 2 3-[3-(tert-Butyl-dimethyl-silanyloxy)-propyl]-6-phenoxy-3H-benzo[c][1,2]-oxaborol-1-ol

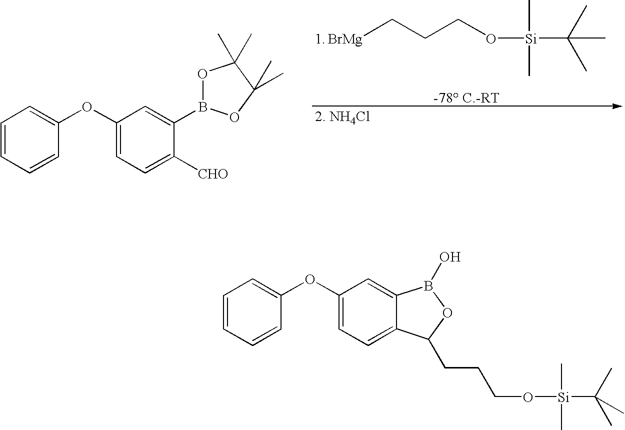

To a solution of 4-phenoxy-2-(4,4,5,5-tetramethyl-[1,3,2]dioxaborolan-2-yl)-benzaldehyde (3.0 g, 9.25 mmol) in anhydrous THF (15 mL) was slowly added the solution of 3-(tert-butyl-dimethyl-silanyloxy)-propylmagnesium bromide (whole from step 1, 13.88 mmol) at −78° C. under nitrogen. The resulting mixture was stirred while slowly warmed to room temperature for 2 h. The mixture was treated with saturated NH₄Cl (50 mL) and extracted with EtOAc. The extract was washed with brine, dried and concentrated to dryness to give the crude product (4.42 g) which could be used without purification. 1HNMR (400 MHz, DMSO-d6) δ 9.12 (s, 1H), 7.42-7.36 (m, 3H), 7.26 (d, J=2.05 Hz, 1H), 7.19-7.13 (m, 2H), 7.03-7.00 (m, 2H), 5.17-14 (m, 1H), 3.60 (t, J=6.5 Hz, 2H), 2.00-1.92 (m, 1H), 1.58-1.46 (m, 3H), 0.85 (s, 9H), 0.05 (s, 6H).

### Step 3 3-(3-Hydroxy-propyl)-6-phenoxy-3H-benzo[c][1,2]oxaborol-1-ol

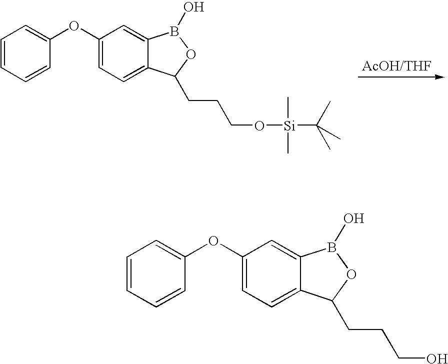

To a solution of 3-[3-(tert-butyl-dimethyl-silanyloxy)-propyl]-6-phenoxy-3H-benzo[c][1,2]-oxaborol-1-ol (4.42 g, crude) in THF (20 mL) was added water (20 mL) and acetic acid (60 mL). The reaction mixture was then stirred at 55-60° C. for 1.5 h. The resulting mixture was concentrated to dryness. The residue was purified by chromatography on silica gel (acetone/hexanes/AcOH=2:5:trace) to give 1.56 g (59%, 2 steps) of pure product as white solid. 1HNMR (400 MHz, DMSO-d6) δ 9.11 (s, 1H), 7.43-7.38 (m, 3H), 7.26 (d, J=2.34 Hz, 1H), 7.11-7.20 (m, 2H), 7.02 (d, J=7.90 Hz, 2H), 5.14 (d, J=6.73 Hz, 1H), 4.41 (t, J=5.27 Hz, 1H), 3.41 (q, J=5.66 Hz, 2H), 1.89-2.02 (m, 1H), 1.38-1.55 (m, 3H). MS (ESI) m/z=283 [M−H]−.

### Step 4 3-(1-Hydroxy-6-phenoxy-1,3-dihydro-benzo[c][1,2]oxaborol-3-yl)-propionic acid

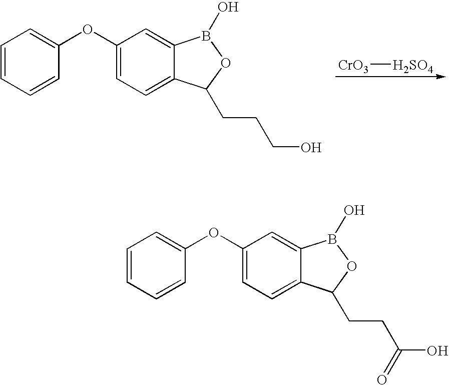

To a solution of 3-(3-hydroxy-propyl)-6-phenoxy-3H-benzo[c][1,2]oxaborol-1-ol (0.5 g, 1.76 mmol) in acetone (15 mL) was added Jones' reagent (½ of the volume of the Jones' reagent of 0.469 g of CrO₃ in 0.5 ml of H₂SO₄ and 1.5 mL of water) at −50° C. The reaction mixture was then stirred at −50° C. to RT for 0.5 h. The resulting mixture was quenched by adding brine at −60° C., extracted with EtOAc. The extract was washed with brine, dried and concentrated to dryness. The residue was purified by chromatography on silica gel (acetone/hexanes/AcOH=1:2:trace) to give 0.30 g of product which was further purified by chromatography on silica gel (MeOH/DCM=3:40) to give 0.203 g of pure product as a white solid; mp 54-56° C. ¹H NMR (400 MHz, DMSO-d6) δ 9.17 (br. s., 1H), 7.32-7.55 (m, 3H), 7.24 (d, J=1.76 Hz, 1H), 7.08-7.20 (m, 2H), 7.01 (d, J=7.91 Hz, 2H), 5.12 (d, J=6.45 Hz, 1H), 2.11-2.40 (m, 3H), 1.63 (q, J=8.60 Hz, 1H). MS (ESI) m/z=297 [M−H]−.

### E40 3-(1-hydroxy-6-(3-hydroxyphenoxy)-1,3-dihydrobenzo[c][1,2]oxaborol-3-yl)propanoic acid

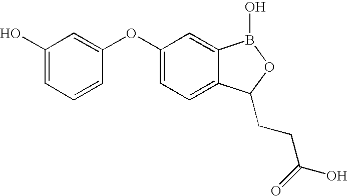

A solution of 3-(6-(3-(benzyloxy)phenoxy)-1-hydroxy-1,3-dihydrobenzo[c][1,2]oxaborol-3-yl)propanoic acid (80 mg, 0.2 mmol) in ethanol was treated with palladium (10% wet on charcoal, 30 mg), then hydrogenation with hydrogen balloon overnight. The mixture was filtered through a Celite pad and rinsed with ethyl acetate. The filtrate was concentrated then purified by column to give product as a white solid (32 mg, 51% yield). This ester was then treated with sodium hydroxide and hydrogen chloride respectively, as demonstrated in the preparations of E55 and E61. ¹H NMR (400 MHz, DMSO-d6) δ 12.0 (b, 1H), 9.56 (s, 1H), 9.14 (s, 1H), 7.36 (d, J=8.4 Hz, 1H), 7.22 (d, J=2.0 Hz, 1H), 7.10 (m, 2H), 6.48 (dd, J=8.0, 2.0 Hz, 1H), 6.36 (dd, J=8, 2.0 Hz, 1H), 6.32 (s, 1H), 5.08 (d, J=8.4 Hz, 1H), 2.20 (m, 2H), 1.60 (m,1H). MS (ESI) m/z=313 [M−H]−.

### E41 N-[2-(1-Hydroxy-6-phenoxy-1,3-dihydro-benzo[c][1,2]oxaborol-3-yl)-acetyl]-methanesulfonamide

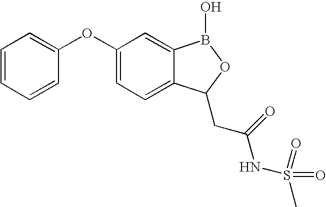

To a suspension of (1-hydroxy-6-phenoxy-1,3-dihydro-benzo[c][1,2]oxaborol-3-yl)-acetic acid (0.15 g, 0.528 mmol) in DCM (5 mL) was added 1,1′-carbonyldiimidazole (0.256 g, 1.58 mmol) at room temperature under nitrogen. The reaction mixture was then stirred at room temperature for 1 h before methanesulfonamide (0.151 g, 1.58 mmol) was added at room temperature. After stirred at room temperature for 0.5 h, 1,8-diazabicyclo[5.4.0]undec-7-ene (0.241 g, 1.58 mmol) was added. Then the resulting mixture was stirred at room temperature for 1 h. The reaction was quenched by adding ice-water and acidified to pH 2 using diluted hydrochloric acid. The mixture was extract with EtOAc. The extract was washed with brine, dried over sodium sulfate, and concentrated. The residue was purified by chromatography on silica gel (DCM/MeOH/AcOH=10:1:trace) to give 0.065 g of pure product as white solid; mp 78-79° C. ¹H NMR (400 MHz, DMSO-d6) δ 9.29 (s, 1H), 7.47 (d, J=8.20 Hz, 1H), 7.38-7.43 (m, 2H), 7.25 (d, J=2.05 Hz, 1H), 7.13-7.22 (m, 2H), 7.01-7.05 (m, 2H), 5.48 (dd, J=9.23, 3.66 Hz, 1H), 3.27 (s, 3H), 2.98 (dd, J=15.38, 3.66 Hz, 1H), 2.41 (dd, 1H) MS (ESI) m/z=360 [M−H]−.

### E42 2-(1-hydroxy-6-(3-hydroxyphenoxy)-1,3-dihydrobenzo[c][1,2]oxaborol-3-yl)acetic acid

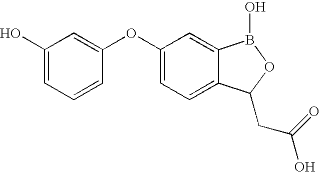

### Step 1. Ethyl 2-(6-(3-(benzyloxy)phenoxy)-1-hydroxy-1,3-dihydrobenzo[c][1,2]oxaborol-3-yl)acetate

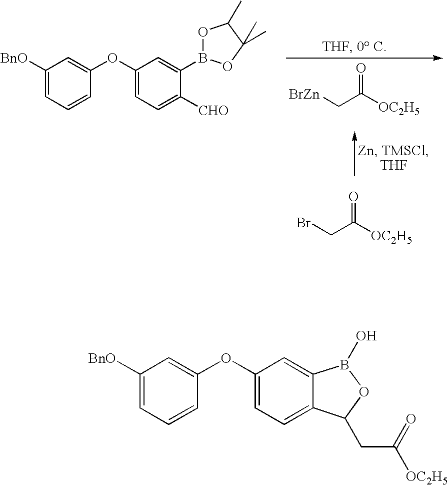

This step was done similarly as Step 1 in the preparation of E90, except using 4-(3-(benzyloxy)phenoxy)-2-(4,4,5,5-tetramethyl-1,3,2-dioxaborolan-2-yl)benzaldehyde (0.86 g, 2 mmol) as starting material. The product is a light yellow oil (0.67 g, 81% yield).

### Step 2. 2-(1-hydroxy-6-(3-hydroxyphenoxy)-1,3-dihydrobenzo[c][1,21]oxaborol-3-yl)acetic acid

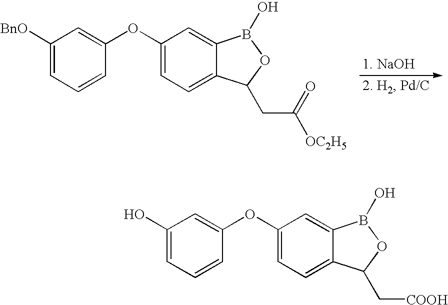

Ethyl 2-(6-(3-(benzyloxy)phenoxy)-1-hydroxy-1,3-dihydrobenzo[c][1,2]oxaborol-3-yl)acetate was treated with sodium hydroxide and hydrogenation respectively, as demonstrated in the preparation of E55 and step 3 of E46. The final product is a white solid. ¹H NMR (400 MHz, DMSO-d6) δ 12.4 (b, 1H), 9.53 (s, 1H), 9.24 (s, 1H), 7.40 (d, J=8.4 Hz, 1H), 7.20 (d, J=2.4 Hz, 1H), 7.10 (m, 2H), 6.47 (dd, J=8, 2 Hz, 1H), 6.36 (dd, J=8, 2 Hz, 1H), 6.30 (t, J=2.4 Hz, 1H), 5.37 (dd, J=9.2, 4 Hz, 1H), 2.87 (dd, J=15.6, 4 Hz, 1H), 2.28 (dd, J=15.6, 8.8 Hz, 1H). MS (ESI) m/z=299 [M−H]+.

### E43 (1-Hydroxy-6-(3-piperazin-1-ylmethyl-phenoxy)-1,3-dihydro-benzo[c][1,2]oxaborol-3-yl)-acetic acid

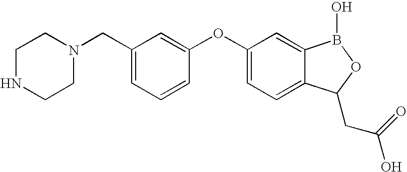

### Step 1: 4-[3-(3-Ethoxycarbonylmethyl-1-hydroxy-1,3-dihydro-benzo[c][1,2]oxaborol-6-yloxy)-benzyl]-piperazine-1-carboxylic acid tert-butyl ester

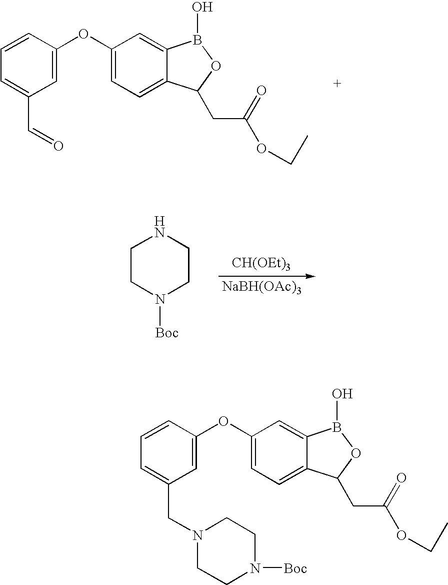

To a solution [6-(3-formyl-phenoxy)-1-hydroxy-1,3-dihydro-benzo[c][1,2]oxaborol-3-yl)-acetic acid ethyl ester (0.8 g, 2.35 mmol) in 1,2-dichloroethane (50 mL) was added two drops of acetic acid, triethylorthoformate (0.69 g, 4.70 mmol) and piperazine-1-carboxylic acid tert-butyl ester (0.87 g, 4.70 mmol). The reaction mixture was stirred at room temperature for 45 minutes. NaBH(OAc)₃ (99 g, 9.4 mmol) was added in portions and the reaction mixture was stirred at room temperature for 3 hours. Aqueous NaOH (1M, 30 mL) was added followed by water (200 mL) and the solution extracted with ethyl acetate (3×200 mL). The organic extracts were combined, dried over Na₂SO₄, filtered and concentrated. The residue was purified by silica gel flash column chromatography (0-7% MeOH/CH₂Cl₂) to give 4-[3-(3-ethoxycarbonylmethyl-1-hydroxy-1,3-dihydro-benzo[c][1,2]oxaborol-6-yloxy)-benzyl]-piperazine-1-carboxylic acid tert-butyl ester as a yellow foam (0.64 g, 53.7%). ¹H NMR 400 MHz (d6-DMSO) δ 9.23 (s, 1H), 7.48 (d, J=8.4 Hz, 1H), 7.36 (m, 1H), 7.24 (d, J=2.0 Hz, 1H), 7.18 (dd, J=8.0, 2.4 Hz, 1H), 7.08 (d, J=7.2 Hz, 1H), 6.96 (s, 1H), 6.90 (dd, J=8.4, 2.0 Hz, 1H), 4.46 (m, 1H), 4.10 (m, 2H), 3.46 (s, 2H), 3.28 (m, 4H), 3.06 (m, 1H), 2.45 (m, 1H), 2.31-2.28 (m, 4H), 1.38 (s, 9H), 1.18 (t, J=6.8 Hz, 3H).

### Step 2: 4-[3-(3-Carboxylmethyl-1-hydroxy-1,3-dihydro-benzo[c][1,2]oxaborol-6-yloxy)-benzyl]-piperazine-1-carboxylic acid tert-butyl ester

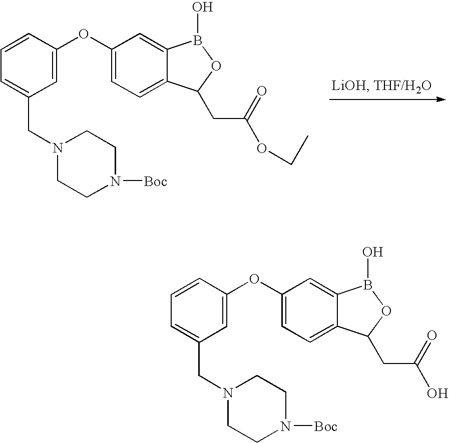

To a solution of 4-[3-(3-ethoxycarbonylmethyl-1-hydroxy-1,3-dihydro-benzo[c][1,2]oxaborol-6-yloxy)-benzyl]-piperazine-1-carboxylic acid tert-butyl ester (0.64 g, 1.25 mmol) in THF (15 mL) at 0° C. was added a solution of LiOH (0.15 g, 6.27 mmol) in water (10 mL). The solution was allowed to warm to room temperature and stirred for 5 hours then acidified to pH 5 with 6M HCl. The solution was extracted with ethyl acetate and the organic extracts washed with water, brine, dried over sodium sulfate and concentrated in vacuo. The residue was purified by silica gel flash column chromatography (1-11% MeOH/DCM) to give 4-[3-(3-carboxylmethyl-1-hydroxy-1,3-dihydro-benzo[c][1,2]oxaborol-6-yloxy)-benzyl]-piperazine-1-carboxylic acid tert-butyl ester as a white solid (0.35 g, 58%). 1HNMR (400 MHz, DMSO-d6) δ 9.19 (s, 1H), 7.48 (d, J=8.4 Hz, 1H), 7.34 (t, J=8.0 Hz, 1H), 7.24 (d, J=2.0 Hz, 1H), 7.17 (d, J=8.0 Hz, 1H), 7.08 (d, J=8.0 Hz, 1H), 6.97 (s, 1H), 6.88 (s, 1H), 5.44 (m, 1H), 4.10 (m, 2H), 3.47 (s, 2H), 3.30 (m, 2H), 2.92 (m, 1H), 2.37-2.28 (m, 5H), 1.38 (s, 9H).

### Step 3: (1-Hydroxy-6-(3-piperazin-1-ylmethyl-phenoxy)-1,3-dihydro-benzo[c][1,2]oxaborol-3-yl)-acetic acid

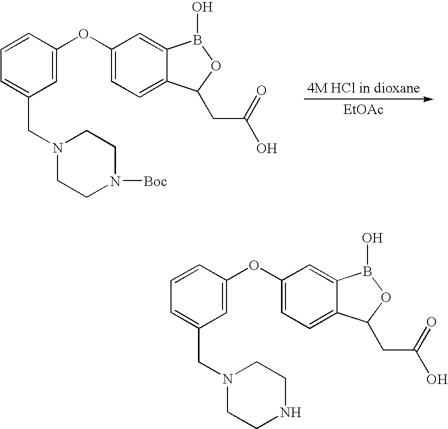

To a solution of 4-[3-(3-carboxylmethyl-1-hydroxy-1,3-dihydro-benzo[c][1,2]oxaborol-6-yloxy)-benzyl]-piperazine-1-carboxylic acid tert-butyl ester (0.32 g, 0.66 mmol) in EtOAc (10 mL) was added 4M HCl in dioxane (1.99 mL, 7.96 mmol) at 0° C. The resulting mixture was stirred at room temperature for 5 hours then concentrated in vacuo. The residue was purified by preparative HPLC to give (1-hydroxy-6-(3-piperazin-1-ylmethyl-phenoxy)-1,3-dihydro-benzo[c][1,2]oxaborol-3-yl)-acetic acid as a white solid (0.145 g, 57.5%). mp 196.2-197.6° C. ¹HNMR (400 MHz, DMSO-d6) δ 7.47 (d, J=8.4, 1H), 7.35 (t, J=8.0, 1H), 7.25 (s, 1H), 7.16 (d, J=8.0, 1H), 7.10 (d, J=7.6, 1H), 6.99 (s, 1H), 6.91 (d, J=7.6, 1H), 5.43 (m, 1H), 3.52 (s, 2H), 3.40-3.20 (m, 6H), 2.99 (s, 2H), 2.93 (m, 1H), 2.35 (m, 1H). MS (ESI) m/z: 383 (M+1)+. HPLC purity: 96.16% (Maxplot), 96.78% (220 nm).

### E44 (1-Hydroxy-6-{3-[(2-methoxy-ethylamino)-methyl]-phenoxy}-1,3-dihydro-benzo[c][1,2]oxaborol-3-yl)-acetic acid

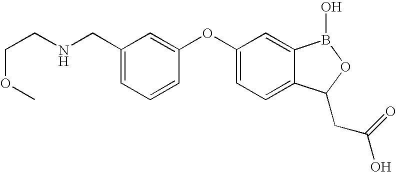

### Step 1: (1-Hydroxy-6-{3-[(2-methoxy-ethylamino)-methyl]-phenoxy}-1,3-dihydro-benzo[c][1,2]oxaborol-3-yl)-acetic acid ethyl ester

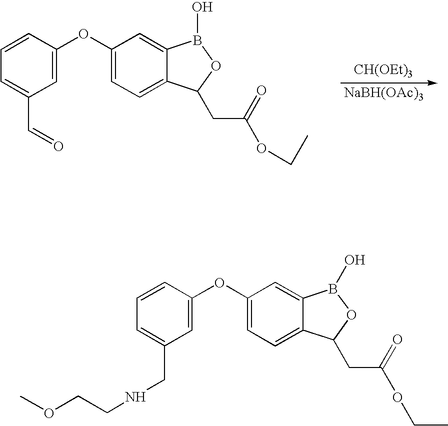

To a solution of [6-(3-formyl-phenoxy)-1-hydroxy-1,3-dihydro-benzo[c][1,2]oxaborol-3-yl)-acetic acid ethyl ester (0.6 g, 1.76 mmol) in 1,2-dichloroethane (40 mL) was added two drops of acetic acid, triethylorthoformate (0.52 g, 3.52 mmol) and 2-methoxyethylamine (0.26 g, 3.52 mmol). The reaction mixture was stirred at room temperature for 45 minutes. NaBH(OAc)₃ (1.49 g, 7.04 mmol) was added in portions and the reaction mixture was stirred at room temperature for 3 hours. Aqueous NaOH (1M, 30 mL) was added followed by water (200 mL) and the solution extracted with ethyl acetate (3×200 mL). The organic extracts were combined, dried over Na₂SO₄, filtered and concentrated. The residue was purified by silica gel flash column chromatography (1-11% MeOH/CH₂Cl₂) to give (1-hydroxy-6-{3-[(2-methoxy-ethylamino)-methyl]-phenoxy}-1,3-dihydro-benzo[c][1,2]oxaborol-3-yl)-acetic acid ethyl ester as a white solid (0.48 g, 68%). ¹H NMR 400 MHz (d6-DMSO) δ 9.25 (s, 1H), 7.45 (d, J=8.4 Hz, 1H), 7.35 (t, J=8.0 Hz, 1H), 7.25 (d, J=2.4 Hz, 1H), 7.20-7.15 (m, 2H), 7.10 (s, 1H), 6.92 (dd, J=8.0, 1.6 Hz, 1H), 5.42 (m, 1H), 4.08 (m, 2H), 3.85 (s, 2H), 3.50 (m, 2H), 3.22 (s, 3H), 3.10 (m, 1H), 2.80 (m, 2H), 2.45 (m, 1H), 1.18 (m, 3H).

### Step 2: (1-Hydroxy-6-{3-[(2-methoxy-ethylamino)-methyl]-phenoxy}-1,3-dihydro-benzo[c][1,2]oxaborol-3-yl)-acetic acid

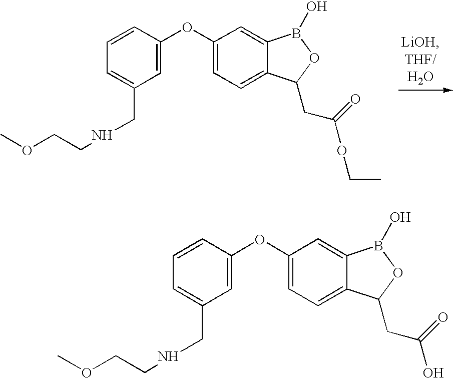

To a solution of (1-hydroxy-6-{3-[(2-methoxy-ethylamino)-methyl]-phenoxy}-1,3-dihydro-benzo[c][1,2]oxaborol-3-yl)-acetic acid ethyl ester (0.26 g, 0.65 mmol) in THF (9 mL) at 0° C. was added a solution of LiOH (0.078 g, 3.26 mmol) in water (5 mL). The solution was allowed to warm to room temperature and stirred for 5 hours then acidified to pH 2 with 6M HCl and concentrated in vacuo. The residue was purified by preparative HPLC to give (1-hydroxy-6-{3-[(2-methoxy-ethylamino)-methyl]-phenoxy}-1,3-dihydro-benzo[c][1,2]oxaborol-3-yl)-acetic acid as a white solid (0.045 g, 18.6%). mp 117.9-118.2° C. 1HNMR (400 MHz, DMSO-d6) δ 7.45 (d, J=8.0 Hz, 1H), 7.34 (t, J=7.6, 1H), 7.22 (d, J=2.4 Hz, 1H), 7.16 (dd, J=8.4, 2.4 Hz, 1H), 7.10 (d, J=7.6 Hz, 1H), 7.00 (s, 1H), 6.85 (d, J=8.0 Hz, 1H), 5.42 (m, 1H), 3.69 (s, 2H), 3.38 (m, 2H), 3.21 (s, 3H), 2.92 (m, 1H), 2.62 (t, J=5.6 Hz, 2H), 2.35 (m, 1H). MS (ESI) m/z: 372 (M+1)+. HPLC purity: 92.93% (Maxplot), 94.31% (220 nm).

### E45 (1-Hydroxy-6-(3-morpholin-4-ylmethyl-phenoxy)-1,3-dihydro-benzo[c][1,2]oxaborol-3-yl)-acetic acid

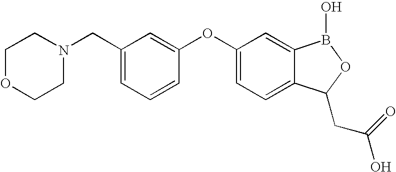

### Step 1: 3-[1,3]Dioxolan-2-yl-phenol

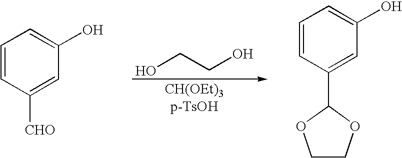

To a solution of 3-hydroxybenzaldehyde (15.0 g, 0.12 mol) and p-toluenesulfonic acid in 1,2-ethanediol (27.2 mL, 0.488 mol)) was added triethylorthoformate (23.66 g, 0.159 mol). The resulting mixture was stirred at room temperature overnight. The reaction mixture was quenched by the addition of aqueous NaHCO₃ (0.1M, 500 mL) and stirred vigorously for 2 minutes at room temperature then extracted with ethyl acetate (3×400 mL). The organic extracts were combined, dried over Na₂SO₄, filtered and concentrated. The residue was purified by silica gel flash column chromatography (10-25% ethyl acetate/hexane) to give 3-[1,3]dioxolan-2-yl-phenol as a clear oil (12.5 g, 61.8%). ¹H NMR 400 MHz (CDCl₃) δ 7.23 (d, J=8.4 Hz, 1H), 7.02 (d, J=8.0 Hz, 1H), 6.94 (d, J=2.0 Hz, 1H), 6.77 (dd, J=7.6, 2.0 Hz, 1H), 5.78 (s, 1H), 5.41 (s, 1H), 4.16-4.07 (m, 2H), 4.06-3.98 (m, 2H).

### Step 2: 2-Bromo-4-(3-[1,3]dioxolan-2-yl-phenoxy)-benzaldehyde

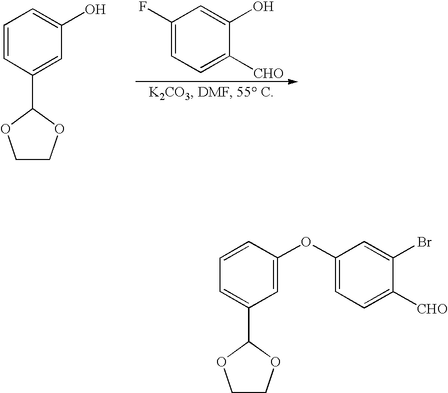

A mixture of 3-[1,3]dioxolan-2-yl-phenol (10.0 g, 60.0 mmol), 2-bromo-4-fluorobenzaldehyde (13.45 g, 66.0 mmol) and K₂CO₃ (12.43 g, 90.0 mmol) in dimethyl formamide (50 mL) was heated at 55° C. overnight. The mixture was cooled to room temperature, diluted with water (1 L) and extracted with 50% ethyl acetate/hexanes (4×600 mL). The organic extracts were combined, dried over Na₂SO₄, filtered and concentrated. The residue was purified by silica gel flash column chromatography (15% ethyl acetate/hexane) to give 2-bromo-4-(3-[1,3]dioxolan-2-yl-phenoxy)-benzaldehyde as a clear oil (19 g, 90%). ¹H NMR 400 MHz (CDCl3) δ 10.25 (s, 1H), 7.89 (d, J=9.2 Hz, 1H), 7.46-7.36 (m, 2H), 7.22 (t, J=2.4 Hz, 1H), 7.18 (d, J=2.4 Hz, 1H), 7.08 (dd, J=2.4, 1.2 Hz, 1H), 6.98 (dd, J=8.4, 2.4 Hz, 1H), 5.82 (s, 1H), 4.14-4.11 (m, 2H), 4.09-4.01 (m, 2H).

### Step 3: 4-(3-[1,3]Dioxolan-2-yl-phenoxy)-2-(4,4,5,5-tetramethyl-[1,3,2]dioxaborolan-2-yl)-benzaldehyde

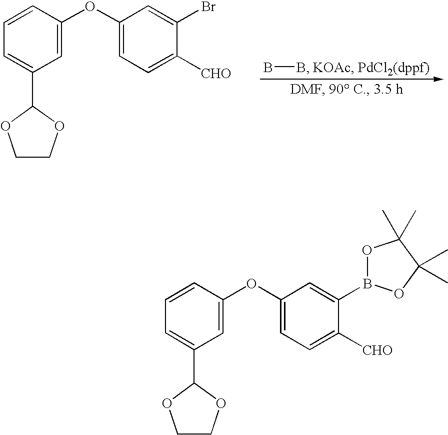

To a mixture of 2-bromo-4-(3-[1,3]dioxolan-2-yl-phenoxy)-benzaldehyde (11.0 g, 31.5 mmol), bispinacolatodiboron (12.0 g, 47.3 mmol) and KOAc (6.2 g, 63 mmol) in dimethylformamide (40 mL) at 90° C., PdCl₂(dppf) (1.15 g, 1.58 mmol) was added and the reaction mixture was stirred at 90° C. for 3.5 hours. After cooling to room temperature, the solution was diluted with water (800 mL) and extracted with ethyl acetate (4×600 mL). The organic extracts were combined, dried over Na₂SO₄, filtered and concentrated. The residue was purified by silica gel flash column chromatography (15-20% ethyl acetate/hexane) to give 4-(3-[1,3]dioxolan-2-yl-phenoxy)-2-(4,4,5,5-tetramethyl-[1,3,2]dioxaborolan-2-yl)-benzaldehyde as a colorless oil (8.0g, 64.5%). ¹H NMR 400 MHz (CDCl₃) δ 10.40 (s, 1H), 7.92 (d, J=8.4 Hz, 1H), 7.44 (d, J=2.4 Hz, 1H), 7.40 (m, 1H), 7.30 (d, J=7.2 Hz, 1H), 7.19 (d, J=2.0 Hz, 1H), 7.05 (m, 2H), 5.80 (s, 1H), 4.13-4.07 (m, 2H), 4.07-4.01 (m, 2H), 1.38 (s, 12H).

### Step 4: [6-(3-[1,3]Dioxolan-2-yl-phenoxy)-1-hydroxy-1,3-dihydro-benzo[c][1,2]oxaborol-3-yl)-acetic acid ethyl ester

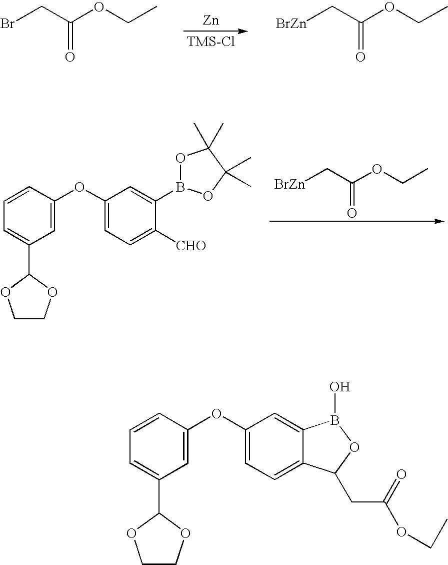

To a suspension of zinc dust (8.17 g, 125 mmol) in THF (60 mL) was added trimethylsilyl chloride (1.8 g, 16.66 mmol) at 40° C. The mixture was heated to 55° C. and stirred for 15 min. After cooling down to 37° C., ethyl bromoacetate (19.47 g, 116.6 mmol) was slowly added to the reaction mixture at 37-40° C. After addition, the resulting mixture was allowed to cool to room temperature over 30 minutes then cooled down to 0° C. 4-(3-[1,3]Dioxolan-2-yl-phenoxy)-2-(4,4,5,5-tetramethyl-[1,3,2]dioxaborolan-2-yl)-benzaldehyde (3.3 g, 8.33 mmol) in THF (30 mL) was added to the zinc solution at 0° C. The mixture was allowed to warm to room temperature over 1.5 hours before treating with saturated NH₄Cl (50 mL) and extracting with EtOAc (10×100 mL). The organic extracts were washed with brine, dried and concentrated in vacuo. The residue was purified by silica gel flash column chromatography (5-100% EtOAc/hexane) to give [6-(3-[1,3]dioxolan-2-yl-phenoxy)-1-hydroxy-1,3-dihydro-benzo[c][1,2]oxaborol-3-yl)-acetic acid ethyl ester (3.0 g, 94%) as a colorless oil. ¹H NMR 400 MHz (CDCl₃) δ 9.26 (s, 1H), 7.48 (d, J=8.4 Hz, 1H), 7.42 (d, J=8.0 Hz, 1H), 7.25-7.18 (m, 3H), 7.04 (m, 2H), 5.71 (s, 1H), 5.47 (m, 1H), 4.12-4.07 (m, 2H), 4.06-4.00 (m, 2H), 3.99-3.90 (m, 2H), 3.07 (m, 1H), 2.46 (m, 1H), 1.20 (m, 3H).

### Step 5: [6-(3-Formyl-phenoxy)-1-hydroxy-1,3-dihydro-benzo[c][1,2]oxaborol-3-yl)-acetic acid ethyl ester

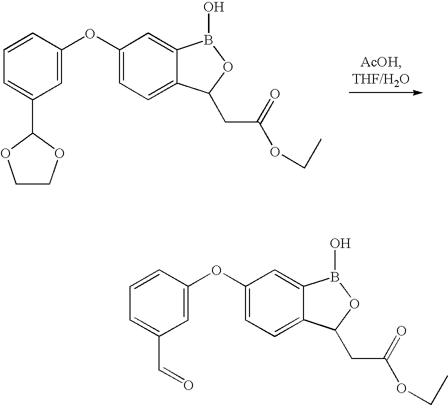

To a solution of [6-(3-[1,3]dioxolan-2-yl-phenoxy)-1-hydroxy-1,3-dihydro-benzo[c][1,2]oxaborol-3-yl)-acetic acid ethyl ester (3.0 g, 7.8 mmol) in tetrahydrofuran (5 mL) and water (5 mL) was added 20 mL of acetic acid. The reaction mixture was heated at 60° C. for 1.5 hours then cooled and concentrated in vacuo. 50 mL of water was added and the solution lyophilized to give [6-(3-formyl-phenoxy)-1-hydroxy-1,3-dihydro-benzo[c][1,2]oxaborol-3-yl)-acetic acid ethyl ester (3.0 g, quantitative). ¹H NMR 400 MHz (CDCl₃) δ]9.96 (s, 1H), 9.25 (s, 1H), 7.69 (d, J=7.6 Hz, 1H), 7.62 (t, J=8.0 Hz, 1H), 7.50 (d, J=8.8 Hz, 1H), 7.41-7.36 (m, 2H), 7.30 (s, 1H), 7.24 (d, J=2.4 Hz, 1H), 5.44 (m, 1H), 4.05 (m, 2H), 3.06 (m, 1H), 2.48 (s, 3H), 2.46 (m, 1H).

### Step 6: [1-Hydroxy-6-(3-morpholin-4-ylmethyl-phenoxy)-1,3-dihydro-benzo[c][1,2]oxaborol-3-yl)-acetic acid ethyl ester

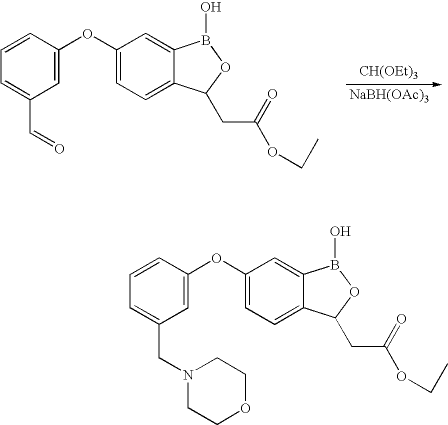

To a solution of [6-(3-formyl-phenoxy)-1-hydroxy-1,3-dihydro-benzo[c][1,2]oxaborol-3-yl)-acetic acid ethyl ester (0.5 g, 1.47 mmol) in 1,2-dichloroethane (30 mL) was added two drops of acetic acid, triethylorthoformate (0.44 g, 2.94 mmol) and morpholine (0.26 g, 2.94 mmol). The reaction mixture was stirred at room temperature for 45 minutes. NaBH(OAc)₃ (1.25 g, 5.88 mmol) was added in portions and the reaction mixture stirred at room temperature for 3 hours. Aqueous NaOH (1M, 30 mL) was added followed by water (200 mL) and the solution extracted with ethyl acetate (3×200 mL). The organic extracts were combined, dried over Na₂SO₄, filtered and concentrated. The residue was purified by Biotage using (1-6% MeOH/CH₂Cl₂) to give [1-hydroxy-6-(3-morpholin-4-ylmethyl-phenoxy)-1,3-dihydro-benzo[c][1,2]oxaborol-3-yl)-acetic acid ethyl ester as a white solid (0.29 g, 48%). 1HNMR (400 MHz, DMSO-d6) δ 9.25 (s, 1H), 7.47 (d, J=8.4 Hz, 1H), 7.34 (t, J=8.0 Hz, 1H), 7.24 (d, J=2.4 Hz, 1H), 7.17 (dd, J=8.0, 2.4 Hz, 1H), 7.08 (d, J=7.6 Hz, 1H), 6.96 (s, 1H), 6.90 (dd, J=8.0, 1.6 Hz, 1H), 5.45 (dd, J=8.4, 3.6 Hz, 1H), 4.07 (m, 2H), 3.55-3.53 (m, 4H), 3.45 (s, 2H), 3.06 (m, 1H), 2.45 (m, 1H), 2.33 (m, 4H). 1.18 (m, 3H).

### Step 7: [1-Hydroxy-6-(3-morpholin-4-ylmethyl-phenoxy)-1,3-dihydro-benzo[c][1,2]oxaborol-3-yl)-acetic acid hydrochloride

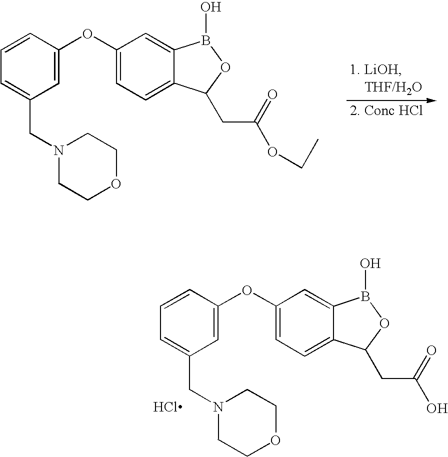

To a solution of [1-hydroxy-6-(3-morpholin-4-ylmethyl-phenoxy)-1,3-dihydro-benzo[c][1,2]oxaborol-3-yl)-acetic acid ethyl ester (0.26 g, 0.63 mmol) in THF (9 mL) was added a solution of LiOH (0.076 g, 3.16 mmol) in water (0.5 mL) at 0° C. The resulting mixture was stirred at room temperature for 5 hours then acidified to pH=2 with 6M hydrochloric acid and concentrated. The residue was purified by silica gel flash column chromatography (5% MeOH/DCM). The obtained product was dissolved in 10 ml of water and 0.5 mL of concentrated HCl and stirred for 10 minutes. The solution was lyophilized to give [1-hydroxy-6-(3-morpholin-4-ylmethyl-phenoxy)-1,3-dihydro-benzo[c][1,2]oxaborol-3-yl)-acetic acid hydrochloride salt as an off white solid. (0.1 g, 38%). mp 137-139.2° C. ¹HNMR (400 MHz, DMSO-d6) δ 10.88 (brs, 1H), 7.50 (d, J=8.4 Hz, 2H), 7.37 (d, J=7.6 Hz, 1H), 7.31 (d, J=2.4 Hz, 2H), 7.22 (m, 1H), 7.12 (m, 1H), 5.44 (m, 1H), 4.33 (s, 2H), 3.93 (d, J=11.6, 2H), 3.74 (t, =12.0 Hz, 2H), 3.22 (d, J=12.0 Hz, 2H), 3.10-3.02 (m, 2H), 2.94 (m, 1H), 2.35 (m, 1H). MS (ESI) m/z: 384 [M+1]+. HPLC purity: 91.09% (Maxplot), 93.48% (220 nm).

### E46 3-(6-(3-(3-aminopropoxy)phenoxy)-1-hydroxy-1,3-dihydrobenzo[c][1,2]oxaborol-3-yl)propanoic acid

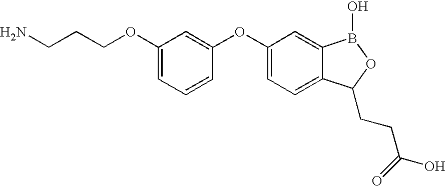

### Step 1. Ethyl 3-(6-(3-(benzyloxy)phenoxy)-1-hydroxy-1,3-dihydrobenzo[c][1,2]oxaborol-3-yl)propanoate

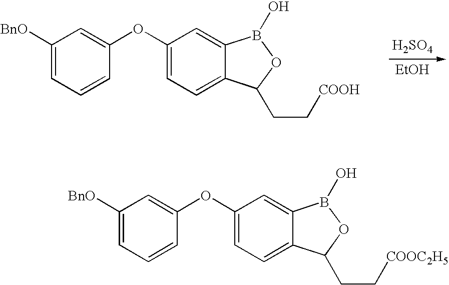

A few drops of concentrated sulfuric acid was added to a solution of 3-(6-(3-(benzyloxy)phenoxy)-1-hydroxy-1,3-dihydrobenzo[c][1,2]oxaborol-3-yl)propanoic acid (0.8 g, 1.98 mmol)in ethanol and the resulting mixture was heated at reflux for two hours. Column purification gave desired product (0.6 g) and recovered starting material.

### Step 2. Ethyl 3-(1-hydroxy-6-(3-hydroxyphenoxy)-1,3-dihydrobenzo[c][1,2]oxaborol-3-yl)propanoate

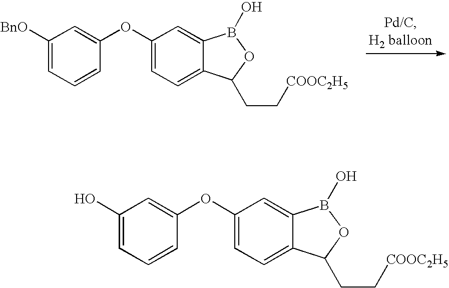

Hydrogenation of ethyl 3-(6-(3-(benzyloxy)phenoxy)-1-hydroxy-1,3-dihydrobenzo[c][1,2]oxaborol-3-yl)propanoate (610 mg, 1.41 mmol) with hydrogen balloon overnight in ethanol with palladium (10% wet on charcoal, 200 mg gave product as a colorless oil (394 mg, 82% yield). MS (ESI) m/z=401 [M−H]+.

### Step 3. 3-(6-(3-(3-aminopropoxy)phenoxy)-1-hydroxy-1,3-dihydrobenzo[c][1,2]oxaborol-3-yl)propanoic acid

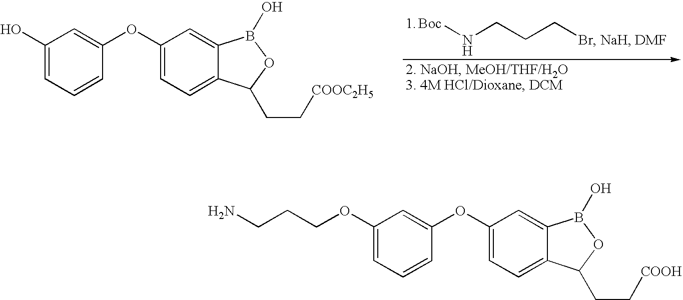

To a cooled (0° C.) suspension of ethyl 3-(1-hydroxy-6-(3-hydroxyphenoxy)-1,3-dihydrobenzo[c][1,2]oxaborol-3-yl)propanoate (103 mg, 0.3 mmol)in DMF was added sodium hydride (40 mg, 0.9 mmol). After stirring at 0° C. for 15 minutes, a solution of tert-butyl 3-bromopropylcarbamate (216 mg, 0.9 mmol) in DMF was added. The reaction was stirred at room temperature for two hours. It was then quenched with water, extracted with EtOAc, washed with brine, dried over Na₂SO₄, and concentrated under reduced pressure. Flash column purification gave colorless oil. This oil was then treated with sodium hydroxide and hydrogen chloride respectively, as demonstrated in the preparation of E55 and E61. The resulting crude is purified by HPLC to give product as a white solid. ¹H NMR (300 MHz, CD₃CN) δ 7.0-7.4 (m, 4H), 6.5-6.7 (m, 3H), 5.2 (d, J=6.3 Hz, 1H), 4.06 (6, J=5.7 Hz, 2H), 3.16 (s, 2H), 2.34 (m, 4H), 2.05 (m, 2H). MS (ESI) m/z=370 [M−H]+.

### E47 3-(6-(3-(benzyloxy)phenoxy)-1-hydroxy-1,3-dihydrobenzo[c][1,2]oxaborol-3-yl)propanoic acid

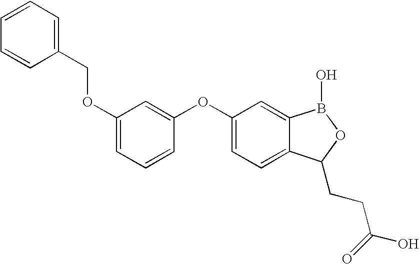

6-(3-(benzyloxy)phenoxy)-3-(3-hydroxypropyl)benzo[c][1,2]oxaborol-1(3H)-ol (1.61 g, 4.13 mmol) was dissolved in 40 ml acetone, cooled to −50° C., then a solution of chromium(VI) oxide (1.1 g, 11 mmol) in sulfuric acid and water (1:3, total 6 ml) was slowly added. The reaction was allowed to warm up to room temperature in 30 minutes. Then it was cooled to −60° C. and quenched with brine, extracted with EtOAc, washed with brine, dried over Na₂SO₄, concentrated under reduced pressure. The crude was purified by column. ¹H NMR (400 MHz, DMSO-d6) δ 12.1 (b, 1H), 9.18 (s, 1H), 7.25-7.5-(m, 8H), 7.14 (dd, J=8.4, 2.4 Hz, 1H), 6.78 (dd, J=8, 2.4 Hz, 1H), 6.30 (t, J=2.4 Hz, 1H), 6.54 (dd, J=8.4, 2.4 Hz, 1H), 5.12(m, 1H), 5.10 (s, 2H), 2.2-2.3 (m, 3H), 1.64 (m, 1H). MS (ESI) m/z=403 [M−H]+.

### E48 2-(6-(3-(3-Aminopropoxy)phenoxy)-1-hydroxy-1,3-dihydrobenzo[c][1,2]oxaborol-3-yl)acetic acid

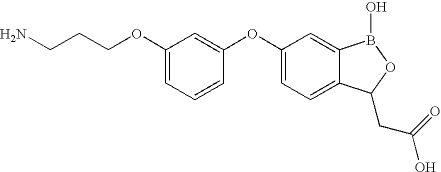

### Step 1. Ethyl 2-(1-hydroxy-6-(3-hydroxyphenoxy)-1,3-dihydrobenzo[c][1,2]oxaborol-3-yl)acetate

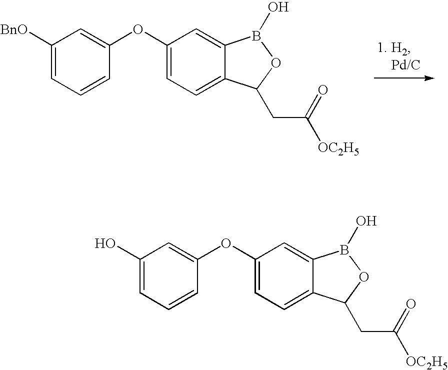

The hydrogenation was done in the same manner as in E40. The product is a colorless oil. MS (ESI) m/z=387 [M−H]+.

### Step 2. 2-(6-(3-(3-Aminopropoxy)phenoxy)-1-hydroxy-1,3-dihydrobenzo[c][1,2]oxaborol-3-yl)acetic acid

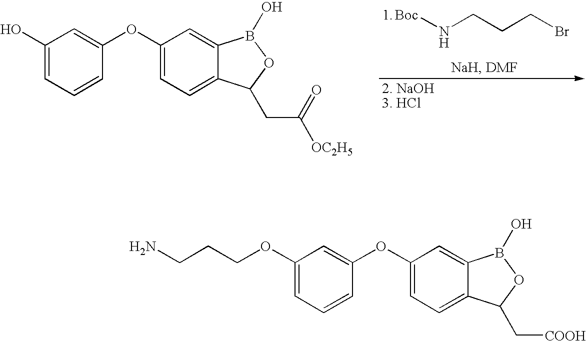

The coupling step and the subsequent two steps were done according to the procedure described in E46. The product is a white powder. MS (ESI) m/z=356 [M−H]+. ¹H NMR (400 MHz, DMSO-d6) δ 12.4 (b, 1H), 9.24 (s, 1H), 7.71 (s, 2H), 7.45 (d, J=8.0 Hz, 1H), 7.26 (t, J=8.0 Hz, 1H), 7.21 (s, 1H), 7.14 (d, J=8.4 Hz, 1H), 6.69 (m, 1H), 6.55 (m, 2H), 5.41 (dd, J=8.8, 4 Hz, 1H), 4.0 (t, J=6 Hz, 2H), 2.93 (m, 3H), 2.30 (dd, J=16, 9.2 Hz, 1H), 1.95 (m, 2H). MS (ESI) m/z=374 [M−H]−.

### E49 (S) {6-[3-(3-Amino-propoxy)-phenoxy]-1-hydroxy-1,3-dihydro-benzo[c][1,2]oxaborol-3-yl}-acetic acid

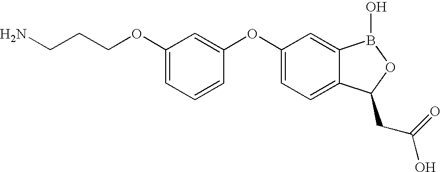

E49 was obtained using a similar procedure as outlined for E50 using (S)-{6-[3-(3-tert-butoxycarbonylamino-propoxy)-phenoxy]-1-hydroxy-1,3-dihydro-benzo[c][1,2]oxaborol-3-yl}-acetic acid ethyl ester.

### E50 (R)-{6-[3-(3-Amino-propoxy)-phenoxy]-1-hydroxy-1,3-dihydro-benzo[c][1,2]oxaborol-3-yl}-acetic acid

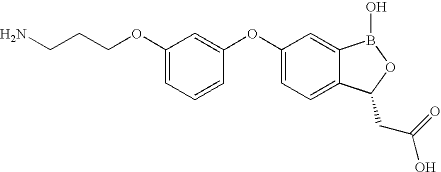

### Step 1: (R)-{6-[3-(3-tert-Butoxycarbonylamino-propoxy)-phenoxy]-1-hydroxy-1,3-dihydro-benzo[c][1,2]oxaborol-3-yl}-acetic acid

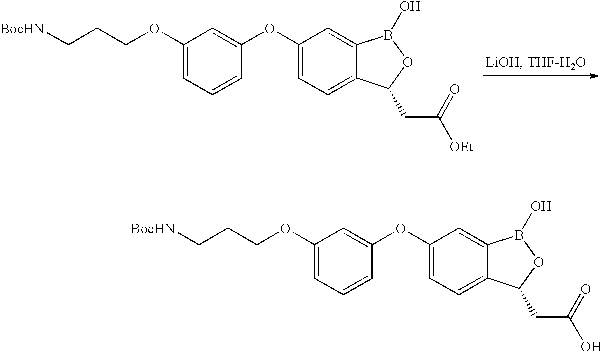

4 g of {6-[3-(3-tert-butoxycarbonylamino-propoxy)-phenoxy]-1-hydroxy-1,3-dihydro-benzo[c][1,2]oxaborol-3-yl}-acetic acid ethyl ester were separated by preparative supercritical fluid chromatography using a CHIRALPAK® IC column (250×50 mm) using a mobile phase composition of 15% methanol in carbon dioxide at a flow rate of 360 ml/min at ambient temperature. The sample size was 20 ml at a concentration of 16.8 g/l, giving a production rate of 1.8 g/hour. The purity of the products was 98.8% ee (1^{st} peak) and 99.2% ee (2^{nd} peak).

To a solution of (R){6-[3-(3-tert-butoxycarbonylamino-propoxy)-phenoxy]-1-hydroxy-1,3-dihydro-benzo[c][1,2]oxaborol-3-yl}-acetic acid ethyl ester (0.860 g, 1.77 mmol) in THF (10 mL) and water (6 mL) was added LiOH (0.26 g, 10.64 mmol) at 0° C. The resulting mixture was stirred at room temperature for 2 hours then cooled to 0° C. and acidified to pH 3 with 6N HCl. The mixture was extracted with EtOAc, dried and concentrated in vacuo to give (R)-{6-[3-(3-tert-butoxycarbonylamino-propoxy)-phenoxy]-1-hydroxy-1,3-dihydro-benzo[c][1,2]oxaborol-3-yl}-acetic acid (0.88 g, quant.). ¹H NMR (400 MHz, MeOD₄) δ 7.40 (d, J=10.2 Hz, 1H), 7.22-7.08 (m, 3H), 6.64 (M, 1H), 6.50 (m, 2H), 5.59 (m, 1H), 3.95 (m, 2H), 3.20 (m, 1H), 2.90 (m, 1H), 2.50 (m, 1H), 1.90 (m, 1H), 1.20 (m, 1H).

### Step 2: (R)-{6-[3-(3-Amino-propoxy)-phenoxy]-1-hydroxy-1,3-dihydro-benzo[c][1,2]oxaborol-3-yl}-acetic acid

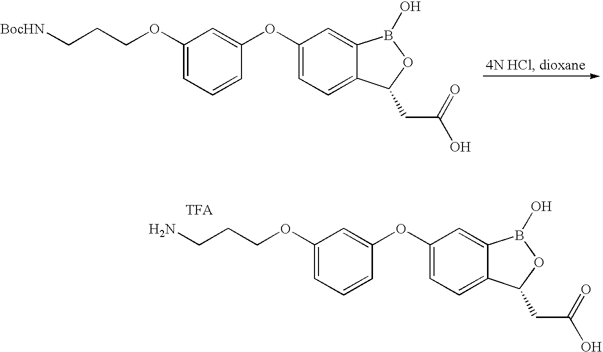

To a solution of (R)-{6-[3-(3-tert-butoxycarbonylamino-propoxy)-phenoxy]-1-hydroxy-1,3-dihydro-benzo[c][1,2]oxaborol-3-yl}-acetic acid (0.88 g, 1.77 mmol) in dioxane (5 mL) was added 4M HCl in dioxane (7.5 mL). The mixture was stirred at room temperature for 1.5 hours and concentrated in vacuo. The residue was purified by preparative HPLC to give {6-[3-(3-amino-propoxy)-phenoxy]-1-hydroxy-1,3-dihydro-benzo[c][1,2]oxaborol-3-yl}-acetic acid (0.580 g, 70%). ¹H NMR (400 MHz, DMSO-d6) δ 9.20 (s, 1H), 8.75 (br s, 3H), 7.45 (d, J=8.8 Hz, 1H), 7.30-7.20 (m, 2H), 7.17 (m, 1H), 6.72 (m, 1H), 6.55 (m, 2H), 5.30 (m, 1H), 4.00 (m, 2H), 2.93 (m, 3H), 2.34 (m, 1H), 1.96 (m, 1H). (MS (ES) m/z: 358 (M+1)+. HPLC purity: 99.51% (220 nm), 98.91% (Maxplot).

### E51 3-(2-Hydroxy-ethyl)-6-phenoxy-3H-benzo[c][1,2]oxaborol-1-ol

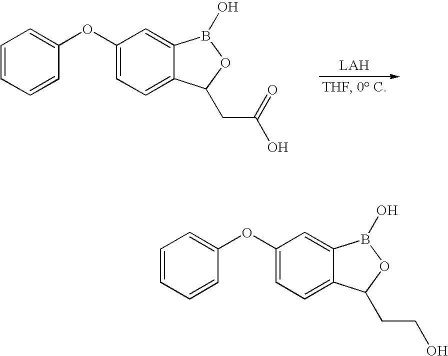

To a solution of (1-hydroxy-6-phenoxy-1,3-dihydro-benzo[c][1,2]oxaborol-3-yl)-acetic acid ethyl ester (0.3 g, 0.960 mmol) in anhydrous THF (10 mL) was slowly added LiAlH₄(0.055 g, 1.44 mmol) at 0° C. under nitrogen. The resulting mixture was stirred at 0° C. to room temperature for 40 min. The reaction was quenched by adding water at 0° C. and the mixture was acidified to pH 2 using diluted hydrochloric acid. The mixture was extracted with EtOAc. The extract was washed with brine and dried to give the crude product which was purified by chromatography on silica gel (acetone/hexanes=1:1). The product was dissolved into water/methanol, acidified to pH 2 using diluted hydrochloric acid and lyophilized to give 0.189 g of pure product as a white powder. Mp 83-85° C. ¹HNMR (400 MHz, DMSO-d₆) δ 9.24 (s, 1H), 7.46 (d, J=8.20 Hz, 1H), 7.39 (t, J=7.76 Hz, 2H), 7.22 (d, J=2.05 Hz, 1H), 7.08-7.19 (m, 2H), 7.00 (d, J=8.20 Hz, 2H), 5.44 (dd, J=8.78, 3.81 Hz, 1H), 4.08 (q, J=7.22 Hz, 2H), 3.64-3.53 (m, 2H), 2.06-2.02 (m, 1H), 1.53-1.47 (m, 1H). MS (ESI) m/z=269 [M−H]⁻.

### E52 3-(3-Hydroxy-propyl)-6-phenoxy-3H-benzo[c][1,2]oxaborol-1-ol

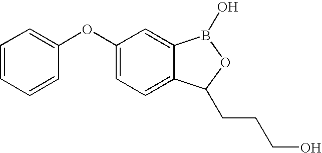

### Step 1. 3-(tert-Butyl-dimethyl-silanyloxy)-propylmagnesium bromide

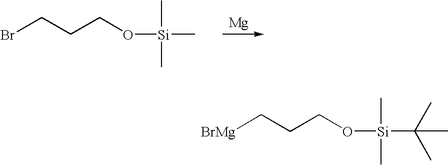

To a mixture of magnesium turnings (0.444 g, 18.3 mmol), iodine (catalytic amount) in anhydrous THF (10 mL) was slowly added (3-bromo-propoxy)-trimethyl-silane (3.56 g, 14.1 mmol) in THF (15 mL) at room temperature under nitrogen. After the reaction initiated, the speed of the addition of the (3-bromo-propoxy)-trimethyl-silane solution was controlled to maintain the temperature of the reaction mixture at 30-35° C. After the addition completed the resulting mixture was stirred at 40° C. for 1.25 h to afford a solution of 3-(tert-butyl-dimethyl-silanyloxy)-propylmagnesium bromide.

### Step 2. 2 3-[3-(tert-Butyl-dimethyl-silanyloxy)-propyl]-6-phenoxy-3H-benzo[c][1,2]-oxaborol-1-ol

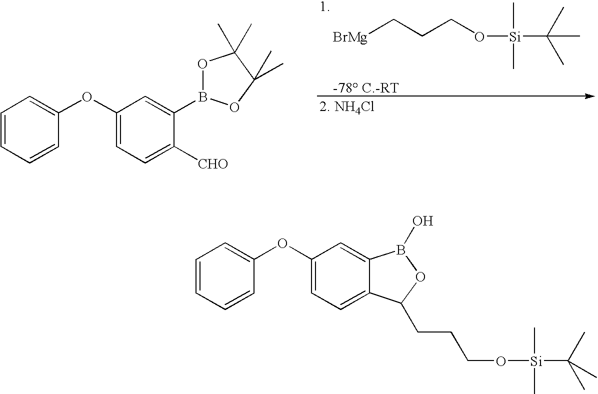

To a solution of 4-phenoxy-2-(4,4,5,5-tetramethyl-[1,3,2]dioxaborolan-2-yl)-benzaldehyde (1.14 g, 3.52 mmol) in anhydrous THF (15 mL) was slowly added the solution of 3-(tert-butyl-dimethyl-silanyloxy)-propylmagnesium bromide (11.5 mL) at −78° C. under nitrogen. The resulting mixture was stirred while slowly warmed to room temperature for 2 h. The mixture was treated with saturated NH₄Cl (50 mL) and extracted with EtOAc. The extract was washed with brine, dried and concentrated to dryness. The residue was purified by chromatography on silica gel (first purification using EtOAc/hexanes=1:5, second purification using THF/hexanes=3:20) to give 0.401 g of product as a colorless oil. ¹HNMR (DMSO-d₆, 400 MHz) δ 9.12 (s, 1H), 7.42-7.36 (m, 3H), 7.26 (d, J=2.05 Hz, 1H), 7.19-7.13 (m, 2H), 7.03-7.00 (m, 2H), 5.17-14 (m, 1H), 3.60 (t, J=6.5 Hz, 2H), 2.00-1.92 (m, 1H), 1.58-1.46 (m, 3H), 0.85 (s, 9H), 0.05 (s, 6H).

### Step 3. 3-(3-Hydroxy-propyl)-6-phenoxy-3H-benzo[c][1,2]oxaborol-1-ol

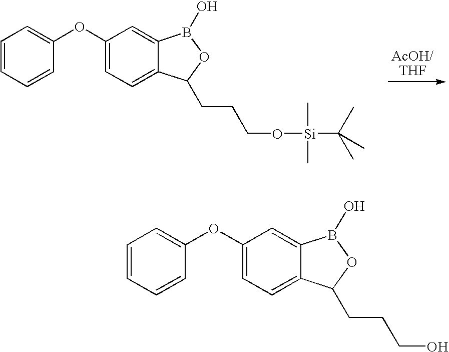

To a solution of 3-[3-(tert-butyl-dimethyl-silanyloxy)-propyl]-6-phenoxy-3H-benzo[c][1,2]-oxaborol-1-ol (0.373 g, 0.936 mmol) in THF (2 mL) was added water (2 mL) and acetic acid (6 mL). The reaction mixture was then stirred at 55-60° C. for 1.5 h. The resulting mixture was concentrated to dryness. The residue was purified by chromatography on silica gel (acetone/hexanes/AcOH=2:5:trace) to give 0.177 g (66.6% yield) of pure product as white solid. Mp 89-91° C. ¹HNMR (DMSO-d₆, 400 MHz) δ 9.11 (s, 1H), 7.43-7.38 (m, 3H), 7.26 (d, J=2.34 Hz, 1H), 7.11-7.20 (m, 2H), 7.02 (d, J=7.90 Hz, 2H), 5.14 (d, J=6.73 Hz, 1H), 4.41 (t, J=5.27 Hz, 1H), 3.41 (q, J=5.66 Hz, 2H), 1.89-2.02 (m, 1H), 1.38-1.55 (m, 3H). MS (ESI) m/z=283 [M−H]⁻.

### E53 6-(3-(benzyloxy)phenoxy)-3-(3-hydroxypropyl)benzo[c][1,2]oxaborol-1(3H)-ol

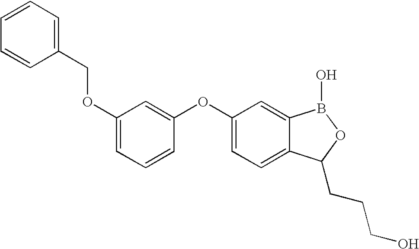

### Step 1. 4-(3-(benzyloxy)phenoxy)-2-bromobenzaldehyde

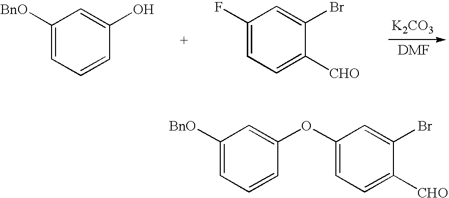

To a mixture of 2-bromo-4-fluoro-benzaldehyde (26.6 g, 131 mmol), 3-(benzyloxy)phenol (25 g, 124.86 mmol) and potassium carbonate (27.1 g, 196 mmol) was added 180 ml DMF. The resulting mixture was heated at 80° C. overnight. The reaction mixture was diluted with EtOAc and washed with water, brine, dried over Na₂SO₄, and concentrated under reduced pressure to give 4-(3-(benzyloxy)phenoxy)-2-bromobenzaldehyde as an off white solid, which was used for the next step without further purification.

### Step 2. 4-(3-(benzyloxy)phenoxy)-2-(4,4,5,5-tetramethyl-1,3,2-dioxaborolan-2-yl)benzaldehyde

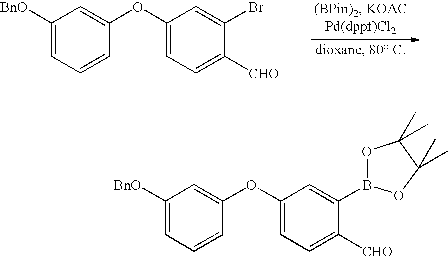

4-(3-(benzyloxy)phenoxy)-2-(4,4,5,5-tetramethyl-1,3,2-dioxaborolan-2-yl)benzaldehyde was made the same way as in Step 2 of the preparation of E35 except using 4-(3-(benzyloxy)phenoxy)-2-bromobenzaldehyde as starting material.

### Step 3. 6-(3-(benzyloxy)phenoxy)-3-(3-(tert-butyldimethylsilyloxy)propyl)benzo[c][1,2]oxaborol-1(3H)-ol

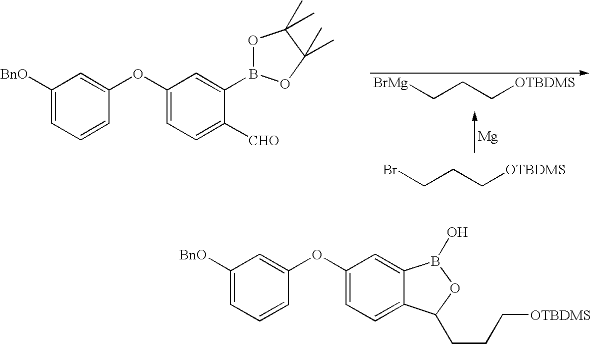

To an oven-dried round bottom flask with stirring bar, Mg turnings (140 mg, 5.76 mmol) and a few 12 crystals was slowly added one-fifth of 3-bromopropoxy)(tert-butyl)dimethylsilane (808 μL, 3.48 mmol) in 5 ml dry THF. This was heated with heat gun until brown color disappeared all at once. Then the rest of 3-bromopropoxy)(tert-butyl)dimethylsilane solution was slowly added to control temperature around 40° C. After the addition completed, the resulting mixture was then heated at 40° C. for one hour. To a cooled (−78° C.) solution of 4-(3-(benzyloxy)phenoxy)-2-(4,4,5,5-tetramethyl-1,3,2-dioxaborolan-2-yl)benzaldehyde in 5 ml dry THF was slowly added the fresh-made Grignard agent. The reaction was allowed to warm up to room temperature and then stirred at room temperature for two hours. The reaction was quenched with saturated ammonium chloride, extracted with EtOAc, washed with brine, dried over Na₂SO₄, and concentrated under reduced pressure. Flash column purification gave product as light yellow oil later solidified to off-white solid (1.04 g, 88% yield).

### Step 4. 6-(3-(benzyloxy)phenoxy)-3-(3-hydroxypropyl)benzo[c][1,2]oxaborol-1(3H)-ol

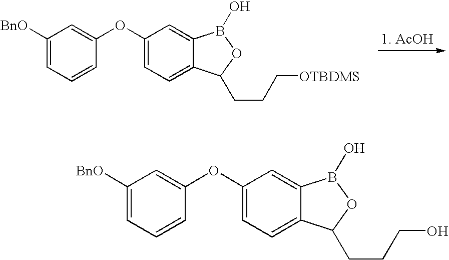

A solution of 6-(3-(benzyloxy)phenoxy)-3-(3-(tert-butyldimethylsilyloxy)propyl)benzo[c][1,2]oxaborol-1(3H)-ol (320 mg, 0.63 mmol) in a mixture of THF:water and acetic acid (1:1:2) was heated at 55° C. for 1.5 hours. The solvent was removed and the crude was purified by column to give 170 mg colorless oil. (69% yield) ¹H NMR (300 MHz, DMSO-d6) δ 9.10 (s, 1H), 7.20-7.40 (m, 8H), 7.13 (dd, J=8.4, 1.8 Hz, 1H), 6.77 (m 1H), 6.63 (m, 1H), 6.52 (dd, J=8.4, 2.4 Hz, 1H), 5.13 (s, 2H), 5.08 (s, 1H), 4.39 (s, 1H), 3.40 (s, 2H), 1.44 (m, 4H).

### E54 3-(Nitromethyl)-6-phenoxybenzo[c][1,2]oxaborol-1(3H)-ol

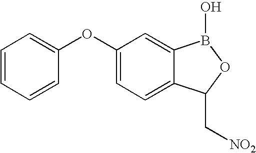

E54 was synthesized by the same method as E56 using 4-phenoxy-2-(4,4,5,5-tetramethyl-1,3,2-dioxaborolan-2-yl)benzaldehyde as starting material instead of 4-(4-formyl-3-(4,4,5,5-tetramethyl-1,3,2-dioxaborolan-2-yl)phenoxy)benzoate. Mp 110-114° C. ¹H NMR (DMSO-d₆, 300 MHz) δ 9.46 (s, 1H), 7.54 (d, J=8.4 Hz, 1H), 7.39 (m, 2H), 7.18 (m, 3H), 7.01 (m, 2H), 5.73 (d, J=8.1, 1H), 5.30 (d, J=12.9 Hz, 1H) and 4.55 (m, 1H). MS (ESI) m/z 284.1 [M−H]⁻

### E55 4-(1-Hydroxy-3-(nitromethyl)-1,3-dihydrobenzo[c][1,2]oxaborol-6-yloxy)benzoic acid

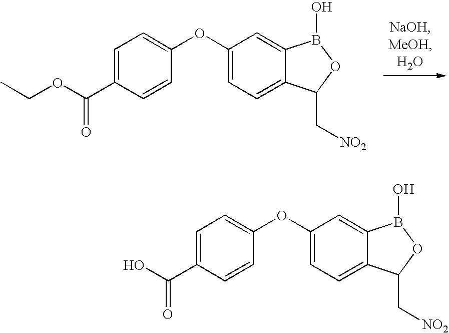

To a solution of ethyl 4-(1-hydroxy-3-(nitromethyl)-1,3-dihydrobenzo[c][1,2]oxaborol-6-yloxy)benzoate (100 mg, 0.28 mmol) in 5 ml of methanol was added 1.5 mL of 1M NaOH aqueous solution. Stir at rt. The reaction was monitored by TLC. After completion, 1 mL of 6 N HCl was added to pH<2 at 0° C. Concentrated, white solid (65 mg) was filtered as target molecule. Mp 141-145° C. ¹H NMR (DMSO-d₆, 300 MHz) δ 12.83 (s, 1H), 9.52 (s, 1H), 7.95 (d, J=8.4 Hz, 2H), 7.62 (d, J=9 Hz, 1H), 7.32 (m, 2H), 7.03 (d, J=8.4 Hz, 2H), 5.78 (d, J=8.1, 1H), 5.36 (d, J=12.9 Hz, 1H) and 4.60 (m, 1H). MS (ESI) m/z 328.1 [M−H]⁻

### E56 Ethyl 4-(1-hydroxy-3-(nitromethyl)-1,3-dihydrobenzo[c][1,2]oxaborol-6-yloxy)benzoate

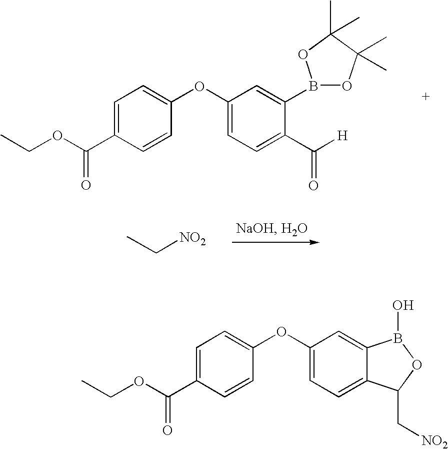

To a solution of NaOH in 10 ml of water was added ethyl 4-(4-formyl-3-(4,4,5,5-tetramethyl-1,3,2-dioxaborolan-2-yl)phenoxy)benzoate by stirring at rt. Stirring continued for 10 min. To the reaction mixture, 2-nitromethane was added dropwise. The solution was stirred for another 30 min. The reaction mixture was cooled to 5° C. and 3N HCl (4 mL) was added dropwise until pH of 2 was attained. Light brown solid precipitated out. Filtered to get 1 g of solid. Then chromatography (H/E 7:3 to 1:1) to get target molecule as off-white solid. ¹H NMR (DMSO-d₆, 300 MHz) δ 9.52 (s, 1H), 7.97 (d, J=8.1 Hz, 2H), 7.62 (d, J=8.7 Hz, 1H), 7.36 (d, J=2.4 Hz, 1H), 7.30 (dd, J=8.4, 2.4 Hz, 1H), 7.06 (d, J=8.4 Hz, 2H), 5.78 (dd, J=9, 2.7 Hz, 1H), 5.35 (dd, J=14.1, 2.7 Hz, 1H), 4.60 (m, 1H), 4.28 (q, 2H) and 1.29 (t, 3H).

### E57 3-(Methylsulfonylmethyl)-6-phenoxybenzo[c][1,2]oxaborol-1(3H)-ol

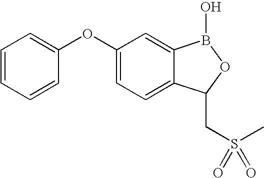

### Step 1. 2-Bromo-4-phenoxybenzaldehyde

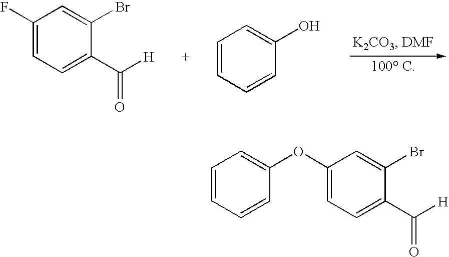

To a mixture of 2-bromo-4-fluorobenzaldehyde (15 g, 73.88 mmol) and phenol (6.95 g, 73.88 mmol) were added dimethylformamide (80 ml) and potassium carbonate (15.32 g, 110.8 mmol). The reaction mixture was heated at 100° C. under a N₂ balloon overnight. After cool down to room temperature, a mixture of ethyl acetate and water was added. After stirring for 20 minutes, the organic layer was separated and the aqueous layer was extracted with more ethyl acetate. The combined organic layer was washed with brine, dried with Na₂SO₄, filtered and evaporated to afford 19.6 g title compound as a slight yellow solid (96% yield). ¹H NMR (DMSO-d₆, 300 MHz) δ ppm 10.1 (s, 1H) 7.86 (d, J=8.7 Hz, 1H) 7.49 (t, J=7.5 Hz, 2H) 7.32-7.27 (m, 2H) 7.18 (d, J=8.7 Hz, 2H) 7.06 (d, J=9.3 Hz, 1H)

### Step 2. 4-Phenoxy-2-(4,4,5,5-tetramethyl-1,3,2-dioxaborolan-2-yl)benzaldehyde

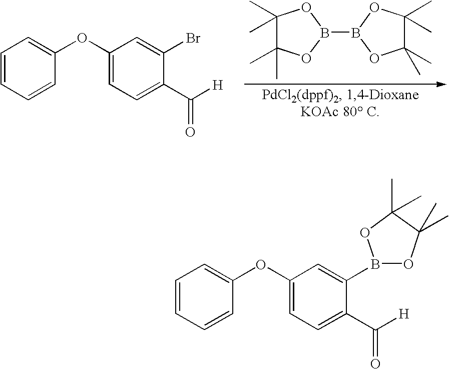

To a mixture of 2-bromo-4-phenoxybenzaldehyde (9.58 g, 34.57 mmol, 1 eq.), Bis(pinacolato)diboron (10.53 g, 41.49 mmol, 1.2 eq) and potassium acetate (10.18 g, 103.7 mmol, 3 eq) was added 1,4-dioxane (140 ml) and 1,1-bis(diphenylphosphino)ferrocene dichloropalladium (706 mg, 0.86 mmol, 2.5mol %). The reaction mixture was de-gassed with N₂ for 20 minutes then heated at 80° C. overnight. After cool down to room temperature, the reaction mixture was filtered through Celite and the Celite cake was washed with more ethyl acetate. The combined filtrate was evaporated and the residue was purified by column chromatography to give product as a light yellow solid. (First batch 4.49 g. NMR good. Second batch 7.72 g. NMR showed impurities but was used in following reactions without any problem.). ¹H NMR (DMSO-d₆, 300 MHz) δ ppm 10.21 (s, 1H) 7.93 (d, J=8.1 Hz, 1H) 7.46 (t, J=8.1 Hz, 2H) 7.25 (t, J=7.7 Hz, 1 H) 7.18-7.11 (m, 4H) 1.29 (s, 12H).

### Step 3. 3-(Methylsulfonylmethyl)-6-phenoxybenzo[c][1,2]oxaborol-1(3H)-ol

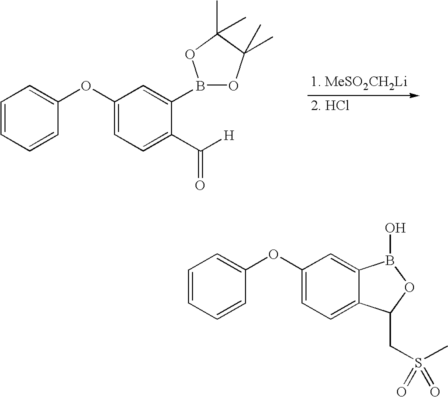

1.6 M n-BuLi in hexanes (2.06 ml, 3.3 mmol) was slowly added to a suspension of dimethylsulfone (438 mg, 4.65 mmol) in 10 ml anhydrous THF. White precipitate crashed out. The reaction was heated at reflux for 1 hour. Then the mixture was cooled down to −78° C., a solution of 4-phenoxy-2-(4,4,5,5-tetramethyl-1,3,2-dioxaborolan-2-yl)benzaldehyde (1.05 g, 3.24 mmol) in 10 ml anhydrous THF was added. The reaction was allowed to slowly warm up to room temperature and stir at room temperature for 30 minutes. 5 ml water was then added and the mixture was acidified to pH 3 with 1N HCl. Then most of the solvent was evaporated by reduced pressure and the residue was extracted with ethyl acetate twice. The combined organic layer was washed with brine, dried over Na₂SO₄, filtered and evaporated. Column purification gave 477 mg product as a white solid (46% yield). ¹HNMR (DMSO-d₆, 300 MHz) δ ppm 9.43 (s, 1H) 7.61 (d, J=8.4 Hz, 1H) 7.40 (t, J=7.1 Hz, 2H) 7.26-7.12 (m, 3H) 7.01 (d, J=8.4 Hz, 2H) 5.46 (d, J=10.2 Hz, 1H) 3.79 (d, J=13.8 Hz, 1H) 3.45-3.37 (m, 1H) 3.08 (s, 3H) MS (ESI) m/z 317 [M−H]⁻.

### E58 3-(Ethylsulfonylmethyl)-6-phenoxybenzo[c][1,2]oxaborol-1(3H)-ol

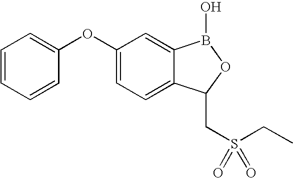

E58 was synthesized by the same method as E57 except using methylethylsulfone as starting material instead of dimethylsulfone. ¹H NMR (DMSO-d₆, 300 MHz) δ ppm 9.42 (s, 1H) 7.62 (d, J=8.4 Hz, 1H) 7.40 (t, J=7.8 Hz, 2H) 7.26-7.14 (m, 3H) 7.01 (d, J=7.5 Hz, 2H) 5.46 (d, J=8.1 Hz, 1H) 3.75 (d, J=13.2 Hz, 1H) 3.44-3.36 (m, 1H) 3.27-3.20 (m, 2H)1.23 (t, 3H). MS (ESI) m/z 331 [M−H]⁻.

E59 (1-Hydroxy-6-phenoxy-1,3-dihydrobenzo[c][1,2]oxaborol-3-yl)methanesulfonic acid

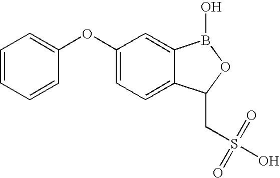

To a solution of 130 mg of methyl (1-hydroxy-6-phenoxy-1,3-dihydrobenzo[c][1,2]oxaborol-3-yl)methanesulfonate (130 mg, 0.39 mmol) in 10 ml acetone was added sodium iodide (64 mg, 0.43 mmol). The mixture was stirred at room temperature overnight and white precipitate crashed out. The precipitate was filtered and washed with more acetone to give produce as a white powder (90 mg, 68% yield). ¹H NMR (300 MHz, DMSO-d6) δ 9.11 (s, 1H), 7.76 (d, J=8.4 Hz, 1H), 7.38 (t, J=7.8 Hz, 2H), 7.14 (m, 3H), 7.0 (d, J=7.2 Hz, 2H), 5.36 (t, J=5.7 Hz, 1H), 2.83 (d, J=5.7 Hz, 2H).

### E60 Methyl (1-hydroxy-6-phenoxy-1,3-dihydrobenzo[c][1,2]oxaborol-3-yl)methanesulfonate

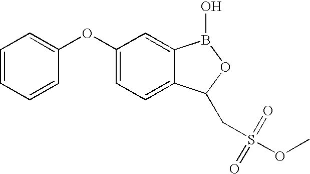

To a cooled (−78° C.) solution of methyl methanesulfonate (304 μL, 3.9 mmol) in 10 ml anhydrous THF was added 1.6 M n-BuLi in hexanes (2.15 ml, 3.45 mmol) dropwise The mixture was stirred at −78° C. for 20 minutes, then a solution of 4-[4-formyl-3-(4,4,5,5-tetramethyl-[1,3,2]dioxaborolan-2-yl)-phenoxy]-piperidine-1-carboxylic acid tert-butyl ester (973 mg, 3 mmol) in 10 mL THF was added slowly via syringe. The reaction mixture was allowed to warm up to 0° C. and stirred at 0° C. for 30 minutes then at room temperature for 1 hour. The reaction was quenched with saturated ammonium chloride, extracted with EtOAc and washed with brine, dried over Na₂SO₄, and concentrated under reduced pressure to give crude product, which was purified by flash column. Recrystallization from hexanes gave product as a white powder. (0.24 g, 72% yield) mp 106-108° C. ¹H NMR (300 MHz, DMSO-d6) δ 9.40 (s, 1H), 7.64 (d, J=8.4 Hz, 1H), 7.40 (t, J=8.4 Hz, 2H), 7.20 (m, 3H), 7.0 (d, J=8.1 Hz, 2H), 5.46 (dd, J=9.6, 1.8 Hz, 1H), 4.10 (dd, J=15, 2.1 Hz, 1H), 3.60 (dd, J=15, 9.6 Hz, 1H), 3.90 (s, 3H). MS (ESI) m/z=333 [M−H]−.

### E61 (1-Hydroxy-6-phenoxy-1,3-dihydrobenzo[c][1,2]oxaborol-3-yl)methanesulfonamide

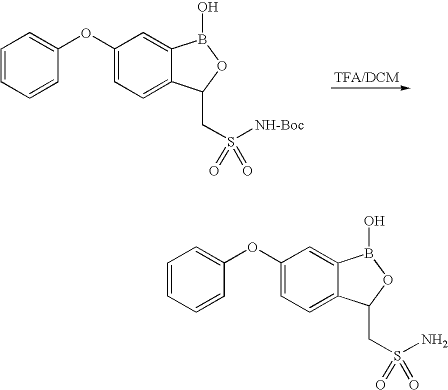

tert-Butyl (1-hydroxy-6-phenoxy-1,3-dihydrobenzo[c][1,2]oxaborol-3-yl)methylsulfonylcarbamate (300 mg, 0.71 mmol) was treated with 25% TFA in DCM for 1 hr. The resulting mixture was evaporated and then co-evaporated with DCM a few times to give title compound as a tan powder. ¹H NMR (DMSO-d₆, 300 MHz) δ ppm 9.36 (s, 1H) 7.53 (d, J=8.7 Hz, 1H) 7.42-7.37 (m, 2H) 7.25-7.15 (m, 3H) 7.02 (dd, J=9.0, 1.2 Hz, 2H) 6.86 (s, 2H) 5.49 (dd, J=14.4, 3 Hz, 1H) 3.67 (dd, J=14.4, 3 Hz, 1H) 3.15 (dd, J=14.7, 8.7 Hz, 1H) MS (ESI) m/z 318 [M−H]⁻.

### E62 N-((1-hydroxy-6-phenoxy-1,3-dihydrobenzo[c][1,2]oxaborol-3-yl)methylsulfonyl)acetamide

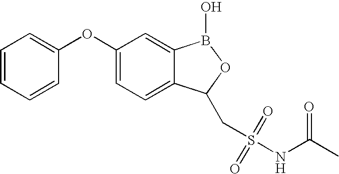

To a solution of (1-hydroxy-6-phenoxy-1,3-dihydrobenzo[c][1,2]oxaborol-3-yl)methane sulfonamide (320 mg, 1 mmol) in pyridine were added acetic anhydride (284 μL, 3 mmol) and DMAP (37 mg, 0.3 mmol). The reaction was allowed to proceed at room temperature with stirring over the weekend. The solvent was removed under reduced pressure. The residue was re-dissolved in EtOAc, washed with water, brine, dried over Na₂SO₄, and concentrated to give crude product, which was purified by flash column. The product was an off-white solid. ¹H NMR (300 MHz, DMSO-d6) δ 11.8 (s, 1H), 9.40 (s, 1H), 7.56 (d, J=8.4 Hz, 1H), 7.38 (t, J=7.8 Hz, 1H), 7.2 (s, 1H), 7.16 (m, 2H), 7.0 (d, J=8.7 Hz, 2H), 5.46 (d, J=7.8 Hz, 1H), 4.10 (d, J=13.8 Hz, 1H), 3.54 (dd, J=14.7, 9.3 Hz, 1H), 3.14 (d, J=3.9 Hz 1H), 2.0 (s, 3H). MS (ESI) m/z=360 [M−H]−.

### E63 N-((1-hydroxy-6-phenoxy-1,3-dihydrobenzo[c][1,2]oxaborol-3-yl)methylsulfonyl)propionamide

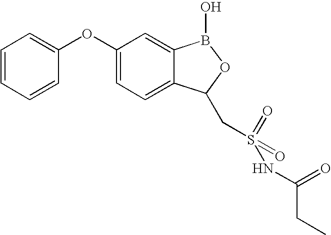

To a cooled (0° C.) solution of propionic acid (336 μL, 4.5 mmol) in DCM was added 1,1′-carbonyldiimidazole (730 mg, 4.5 mmol) and the mixture was stirred at room temperature for two hours. (1-Hydroxy-6-phenoxy-1,3-dihydrobenzo[c][1,2]oxaborol-3-yl)methanesulfonamide (480 mg, 1.5 mmol) and 1,8-diazabicyclo[5.4.0]undec-7-ene (673 μL, 4.5 mmol) were then added. The reaction was stirred at room temperature overnight. The reaction was quenched with water, extracted with EtOAc and washed with saturated ammonium chloride, brine, dried over Na₂SO₄, and concentrated under reduced pressure. Flash column purification gave product as pale yellow solid. ¹H NMR (300 MHz, DMSO-d6) δ 12.0 (b, 1H), 9.12 (s, 1H), 6.90-7.70 (m, 8H), 5.38 (m, 1H), 4.05 (m, 1H), 3.18 (m, 1H), 2.0(q, J=7.8 Hz, 2H), 0.9 (t, J=7.5 Hz, 3H). MS (ESI) m/z=374 [M−H]−.

### E64 N-((1-hydroxy-6-phenoxy-1,3-dihydrobenzo[c][1,2]oxaborol-3-yl)methylsulfonyl)cyclopropanecarboxamide

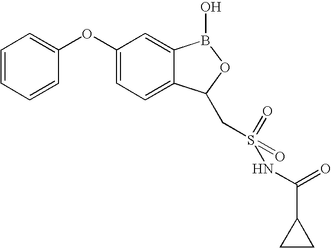

This was made in the same manner as E63 using cyclopropane carboxylic acid as starting material. ¹H NMR (300 MHz, DMSO-d6) δ 12.06 (s, 1H), 9.38 (s, 1H), 7.50 (d, J=8.1 Hz, 1H), 7.38 (t, J=7.8 Hz, 2H), 7.28 (s, 1H), 7.19 (m, 2H), 7.0 (d, J=8.1 Hz, 2H), 5.46 (d, J=7.8 Hz, 1H), 4.10 (m, 2H), 1.76 (m, 1H), 0.84 (m, 4H). MS (ESI) m/z=386 [M−H]−.

### E65 (1-Hydroxy-6-phenoxy-1,3-dihydrobenzo[c][1,2]oxaborol-3-yl)methanesulfonamide

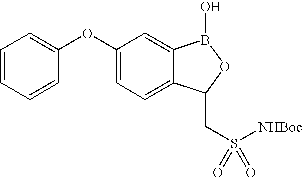

### Step 1. tert-Butyl methylsulfonylcarbamate

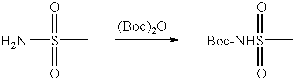

To a stirred suspension of methylsulfonamide (6 g, 62 mmol) in DCM at 0° C. was added DMAP (760 mg, 6.2 mmol), triethylamine (10.4 ml, 74.4 mmol) and (Boc)₂O (14.2 g, 65.1 mmol). The reaction mixture was warmed up to room temperature and stirred overnight. The solution was concentrated and the residue was diluted with ethyl acetate, washed consecutively with 1N HCl and water, dried with Na₂SO₄, filtered and evaporated to afford a colorless oil. The oil was refluxed in hexane for 1 hour then cooled to room temperature and filtered to afford the target compound as a white solid (12.1 g, 42.1% yield). ¹H NMR (DMSO-d₆, 300 MHz) δ ppm 11.22 (s, 1H), 3.18 (s, 3H), 1.42 (s, 9H).

### Step 2. tert-Butyl (1-hydroxy-6-phenoxy-1,3-dihydrobenzo[c][1,2]oxaborol-3-yl)methylsulfonylcarbamate

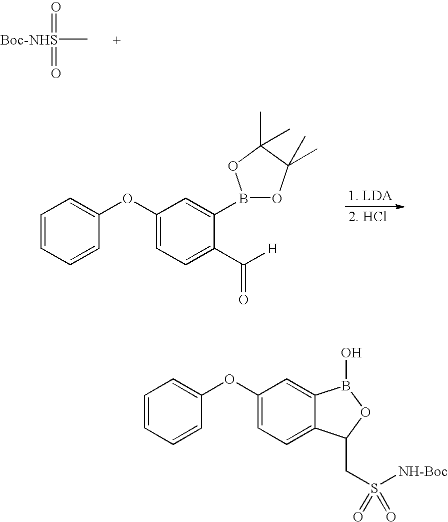

1.6 M n-Butyl lithium in hexanes (20.2 ml, 32.4 mmol) was added drop wise to a solution of N,N-diisopropylethylamine (5.91 ml, 33.9 mmol) in 25 ml dry THF in ice bath and stirred for 30 minutes. Then tert-butyl methylsulfonylcarbamate in 25 ml dry THF was added slowly and stirred at 0° C. for 1 hour. The mixture was then cooled to −78° C. and 4-phenoxy-2-(4,4,5,5-tetramethyl-1,3,2-dioxaborolan-2-yl)benzaldehyde (3.01 g, 15.4 mmol) in 25 ml dry THF was added slowly. The reaction was slowly warmed up to room temperature and stirred at room temperature for 1 hour. Then it was quenched with water, acidified with 1N HCl to pH 3, extracted with ethyl acetate. The combined organic layer was then dried over Na₂SO₄, filtered and concentrated. Column purification gave 1 g product as off-white solid. ¹H NMR (DMSO-d₆, 300 MHz) δ ppm 11.34 (s, 1H), 9.39 (s, 1H), 7.54 (d, J=8.10 Hz, 1H), 7.42-7.37 (m, 2H), 7.28 (d, J=2.40, 1H), 7.22-7.12 (m, 2H), 7.03-7.01 (m, 2H), 5.43 (dd, J=9.3, 1.8 Hz, 1H), 4.01 (dd, J=13.5, 2.1 Hz, 1H), 3.54 (dd, J=14.7, 9.3 Hz, 1H), 1.42 (s, 9H). MS (ESI) m/z 418 [M−H]⁻.

### E66 (1-Hydro-6-phenoxy-1,3-dihydrobenzo[c][1,2]oxaborol-3-ylmethyl)-phosphonic acid

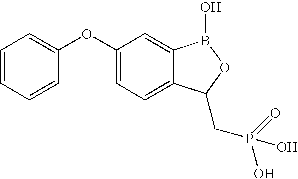

To a solution of (1-hydro-6-phenoxy-1,3-dihydrobenzo[c][1,2]oxaborol-3-ylmethyl)-phosphonic acid dimethyl ester (0.088 g, 0.25 mmol) was added TMSI at 0° C. and stirred for 30 minutes. MeOH was added and the reaction stirred at room temperature for 30 minutes then concentrated in vacuo. A second portion of MeOH was added and the solution concentrated. The residue was purified by preparative HPLC to give (1-hydro-6-phenoxy-1,3-dihydrobenzo[c][1,2]oxaborol-3-ylmethyl)-phosphonic acid (0.035 g, 44%). mp: 143-145° C. ¹H NMR (400 MHz, DMSO-d₆) δ: 9.20 (s, 1H), 7.62 (d, J=8.3 Hz, 1H), 7.40 (m, 2H), 7.23 (s, 1H), 7.22-7.17 (m, 2H), 7.00 (m, 2H), 5.27 (m, 1H), 2.10-2.00 (m, 1H), 2.00-1.80 (m, 1H). MS (ESI) m/z: 319 [M−1]⁻; HPLC purity: 98.48% (220 nm), 97.51% (Maxplot).

### E67 (1-Hydro-6-phenoxy-1,3-dihydrobenzo[c][1,2]oxaborol-3-ylmethyl)-phosphonic acid dimethyl ester

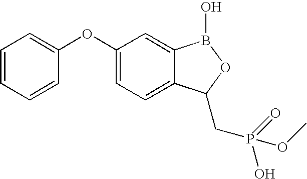

A solution of (1-hydro-6-phenoxy-1,3-dihydrobenzo[c][1,2]oxaborol-3-ylmethyl)-phosphonic acid dimethyl ester (0.095 g, 0.27 mmol) in MeOH (3 mL) and 6 N HCl (3 mL) was refluxed for 48 hours then concentrated in vacuo. The residue was purified by silica gel flash column chromatography to give (1-hydro-6-phenoxy-1,3-dihydrobenzo[c][1,2]oxaborol-3-ylmethyl)-phosphonic acid monomethyl ester (0.018 g, 20%). mp 143-145° C. ¹H NMR (400 MHz, DMSO-d₆) δ: 9.25 (s, 1H), 7.60 (d, J=8.3 Hz, 1H), 7.40 (m, 2H), 7.23 (s, 1H), 7.22-7.17 (m, 2H), 7.00 (m, 2H), 5.35 (m, 1H), 3.65 (d, J=8.4 Hz, 3H), 2.40 (m, 1H), 2.00 (m, 1H). MS (ESI) m/z: 333 [M−H]⁻. HPLC purity: 98.13% (220 nm), 94.15% (254 nm), 97.71% (Maxplot).

### E68 (1-Hydro-6-phenoxy-1,3-dihydrobenzo[c][1,2]oxaborol-3-ylmethyl)-phosphonic acid dimethyl ester

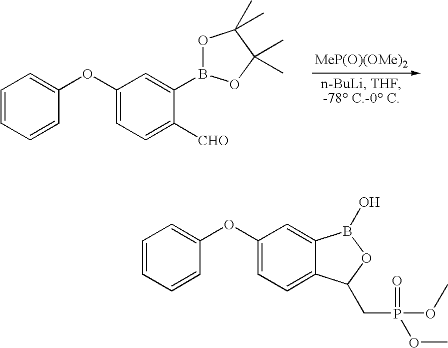

To a cooled (−78° C.) solution of methyl phosphonic acid dimethylester (0.16 g, 1.30 mmol) in THF was added n-BuLi (0.46 mL, 2.5 M in Hexane) dropwise. After the addition was over, the mixture was stirred at −78° C. for 15 min, a solution of 4-phenoxy-2-(4,4,5,5-tetramethyl-[1,3,2]dioxaborolan-2-yl)benzaldehyde (324 mg, 1 mmol) in THF (4 mL) was added via syringe. The resulting mixture was gradually warmed up to 0° C., and kept at 0° C. for 20 min, then quenched with saturated aqueous NH₄Cl, extracted with EtOAc, dried and concentrated. The residue was purified by chromatography to give (1-hydro-6-phenoxy-1,3-dihydrobenzo[c][1,2]oxaborol-3-ylmethyl)-phosphonic acid dimethyl ester (190 mg, 52% yield) as a white solid. Mp 143-145° C. ¹H NMR (DMSO-d₆, 400 MHz) δ 9.25 (s, 1H), 7.60 (d, J=8.3 Hz, 1H), 7.40 (m, 2H), 7.23 (s, 1H), 7.22-7.17 (m, 2H), 7.00 (m, 2H), 5.25 (m, 1H), 3.65 (m, 6H), 2.70-2.50 (m, 1H), 2.10-2.00 (m, 1H). MS (ESI) m/z=249 [M+H]⁺.

### E69 6-Phenoxy-3-[1,2,3]triazol-2-ylmethyl-3H-benzo[c][1,2]oxaborol-1-ol

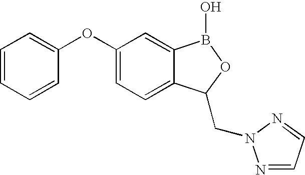

To a solution of 1-(2-bromo-4-phenoxy-phenyl)-2-[1,2,3]triazol-2-yl-ethanol (0.44 g, 1.22 mmol) in toluene (30 mL) was added triisopropyl borate (0.459 g, 2.44 mmol) under nitrogen. The reaction mixture was then stirred at reflux and toluene was slowly distilled out. The resulting mixture was dissolved into THF (10 mL). BuLi (2.5 M in hexane, 0.59 mL, 1.46 mmol) was added to the reaction mixture at −78° C. and stirred at this temperature for 2 h while the temperature was slowly warmed up to room temperature. Then the reaction was quenched by adding water at 0° C. and acidified to pH 2 using diluted hydrochloric acid. The mixture was extract with EtOAc. The extract was washed with brine, dried over sodium sulfate, and concentrated. The residue was purified by chromatography on silica gel (EtOAc/hexanes/AcOH=1:2:trace) to give 0.101 g material which was purified again by prep-HPLC to give 0.052 g of pure product as white solid; mp 43-45° C. ¹H NMR (400 MHz, DMSO-d₆) δ 9.30 (s, 1H), 7.77 (s, 2H), 7.34-7.42 (m, 3H), 7.21 (d, J=2.34 Hz, 1H), 7.11-7.19 (m, 2H), 6.97-7.02 (m, 2H), 5.63 (dd, J=7.77, 3.96 Hz, 1H), 4.93 (dd, J=13.92, 3.96 Hz, 1H), 4.55 (dd, J=14.07, 7.91 Hz, 1H). MS (ESI) m/z=308 [M +H]⁺.

### E70 6-Phenoxy-3-[1,2,3]triazol-1-ylmethyl-3H-benzo[c][1,2]oxaborol-1-ol

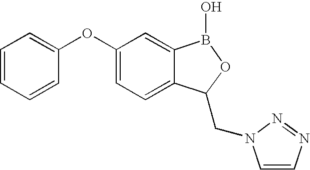

### Step 1 2-(2-Bromo-4-phenoxy-phenyl)-oxirane

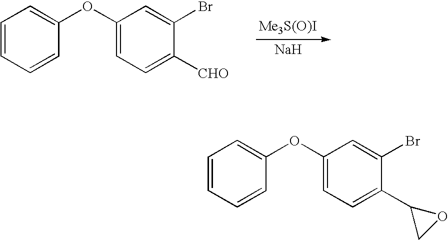

To a suspension of NaH (95%, 0.656 g, 26.0 mmol) in dry DMSO (40 mL) was slowly added trimethylsulfoxoium iodide (5.718 g, 26.0 mmol) at 10-20° C. The mixture was stirred at room temperature till no gas released. A solution of 2-bromo-4-phenoxy-benzaldehyde (6.0 g, 21.7 mmol) in dry DMSO (15 mL) was added to the reaction mixture at 10-20° C. Then the resulting mixture was stirred at room temperature for 2 h. The reaction mixture was poured into ice-water (100 mL), extracted with EtOAc. The extract was washed with brine, dried over sodium sulfate, and concentrated. The residue was purified by chromatography on silica gel (EtOAc/hexanes=1:20) to give 3.62 g of pure product as colorless oil. ¹HNMR (400 MHz, CDCl₃) δ 7.37 (dd, J=8.64, 7.47 Hz, 2H), 7.12-7.20 (m, 3H), 7.00-7.04 (m, 2H), 6.94 (dd, J=8.79, 2.64 Hz, 1H), 4.12 (dd, J=4.10, 2.34 Hz, 1H), 3.17 (dd, J=5.57, 4.10 Hz, 1H), 2.66 (dd, 1H).

### Step 2 1-(2-Bromo-4-phenoxy-phenyl)-2-[1,2,3]triazol-1-yl-ethanol and 1-(2-bromo-4-phenoxy-phenyl)-2-[1,2,3]triazol-2-yl-ethanol

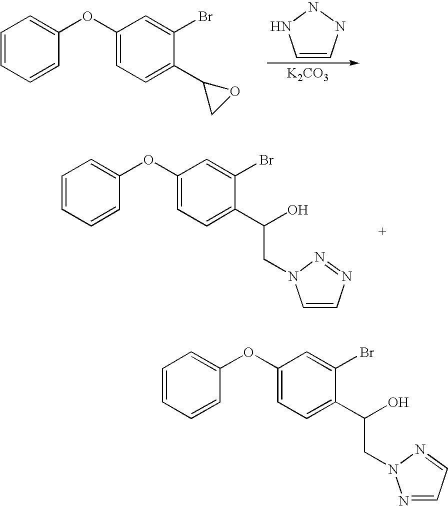

To a solution of 2-(2-bromo-4-phenoxy-phenyl)-oxirane (1.5 g, 5.15 mmol), 1H-1,2,3-triazole (1.103 g, 15.97 mmol) in anhydrous DMF (10 mL) was added potassium carbonate (2.207 g, 15.97 mmol) at room temperature under nitrogen. The resulting mixture was stirred at 80° C. for 3 h. The reaction mixture was poured into ice-water (20 mL), acidified to pH 2 using diluted hydrochloric acid, and extracted with EtOAc. The extract was washed with brine, dried over sodium sulfate, and concentrated. The residue was purified by chromatography on silica gel (EtOAc/hexanes=1:1) to give 1.07 g of 1-(2-bromo-4-phenoxy-phenyl)-2-[1,2,3]triazol-1-yl-ethanol as colorless oil, 1HNMR (400 MHz, DMSO-d6) δ 8.04 (s, 1H), 7.71 (s, 1H), 7.52 (d, J=8.59 Hz, 1H), 7.44 (t, J=7.81 Hz, 2H), 7.18-7.25 (m, 2H), 7.01-7.10 (m, 3H), 6.01 (d, J=4.68 Hz, 1H), 5.16-5.23 (m, 1H), 4.59 (dd, J=13.85, 3.32 Hz, 1H), 4.42 (dd, 1H), and 0.460 g of 1-(2-bromo-4-phenoxy-phenyl)-2-[1,2,3]triazol-2-yl-ethanol as colorless oil, 1HNMR (400 MHz, DMSO-d6) δ 7.77 (s, 2H), 7.63 (d, J=8.98 Hz, 1H), 7.44 (t, J=7.81 Hz, 2H), 7.17-7.25 (m, 2H), 7.07 (d, J=7.81 Hz, 3H), 5.84 (d, J=5.07 Hz, 1H), 5.40 (m, 1H), 4.50-4.57 (m, 1H), 4.42-4.49 (m, 1H)

### Step 3 6-Phenoxy-3-[1,2,3]triazol-1-ylmethyl-3H-benzo[c][1,2]oxaborol-1-ol

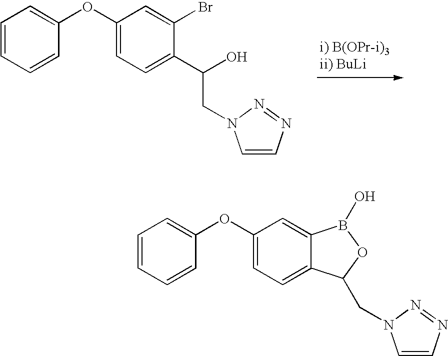

To a solution of 1-(2-bromo-4-phenoxy-phenyl)-2-[1,2,3]triazol-1-yl-ethanol (0.6 g, 1.66 mmol) in toluene (15 mL) was added triisopropyl borate (0.626 g, 3.33 mmol) under nitrogen. The reaction mixture was then stirred at 90° C. for 0.5 h and then toluene was slowly distillated out. The resulting mixture was dissolved into THF (10 mL). BuLi (2.5 M in hexane, 0.87 mL, 2.16 mmol) was added to the reaction mixture at −78° C. and stirred at this temperature for 20 min before the temperature was slowly warmed up to room temperature for 2 h. Then the reaction was quenched by adding water at 0° C. and acidified to pH 2 using diluted hydrochloric acid. The mixture was extract with EtOAc. The extract was washed with brine, dried over sodium sulfate, and concentrated. The residue was purified by chromatography on silica gel (EtOAc/hexanes/AcOH=2:1:trace) to give 0.102 g material which was recrystallized from EtOAc/hexanes to give 0.052 g of pure product as white solid; mp 158-159° C. 1HNMR (400 MHz, DMSO-d6) δ 9.35 (s, 1H), 7.98 (s, 1H), 7.69 (s, 1H), 7.53 (d, J=9.08 Hz, 1H), 7.41 (t, J=8.05 Hz, 2H), 7.13-7.25 (m, 3H), 7.01 (d, J=7.90 Hz, 2H), 5.56 (dd, J=7.47, 3.37 Hz, 1H), 4.99 (dd, J=14.20, 3.37 Hz, 1H), 4.57 (dd, 1H). MS (ESI) m/z=308 [M+H]+.

### E71 Ethyl 1-hydroxy-6-phenoxy-1,3-dihydrobenzo[c][1,2]oxaborole-3-carboxylate

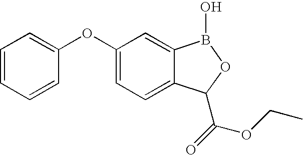

### Step 1. 1-Hydroxy-6-phenoxy-1,3-dihydrobenzo[c][1,2]oxaborole-3-carbonitrile

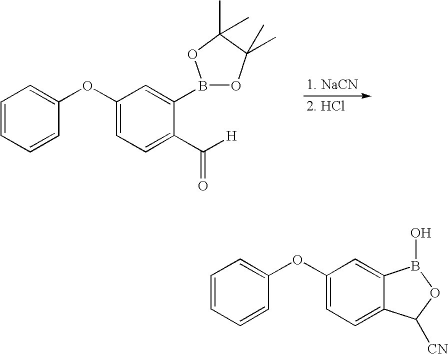

To a solution of sodium cyanide (98 mg, 2 mmol, 1 eq) in 5 ml of water was added 4-phenoxy-2-(4,4,5,5-tetramethyl-1,3,2-dioxaborolan-2-yl)benzaldehyde (650 mg, 2 mmol, 1 eq) in 5 ml THF. After stirring for 30 minutes at room temperature, the solution was acidified with 1N HCl to pH 3. The mixture was then extracted with ethyl acetate. The combined organic layer was dried over Na₂SO₄, filtered and evaporated to get 560 mg off-white solid. MS (ESI(−)) m/z 250 [M−H]⁻.

### Step 2. Ethyl 1-hydroxy-6-phenoxy-1,3-dihydrobenzo[c][1,2]oxaborole-3-carboxylate

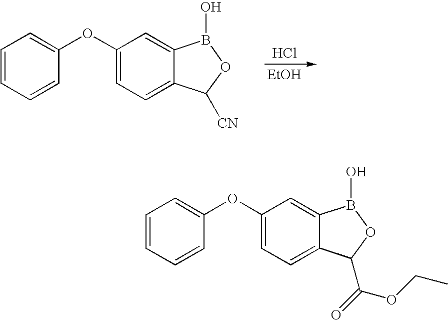

1-Hydroxy-6-phenoxy-1,3-dihydrobenzo[c][1,2]oxaborole-3-carbonitrile was dissolved in a mixture of 5 ml ethanol and 5 ml 6N HCl, and heated at 80° C. for 3.5 hours, 1 ml concentrated HCl was then added and the reaction was allowed to stir at room temperature overnight. Column chromatography gave 170 mg target compound as colorless oil (yield for the two steps: 31%). ¹H NMR (DMSO-d₆, 300 MHz) δ ppm 9.61 (s, 1H) 7.50 (d, J=8.1 Hz, 1H) 7.43-7.37 (m, 2H) 7.27-7.15 (m, 3H) 7.03 (d, J=7.8 Hz, 2H) 5.72 (s, 1H) 4.16 (q, J=5.4 Hz, 2H) 1.21 (t, 3H) MS (ESI(−)) m/z 297 [M−H]⁺.

### E72 (1-Hydroxy-6-pyridin-3-yoxyl)-1,3-dihydro-benzo[c][1,2]oxaborol-3-yl)-acetic acid

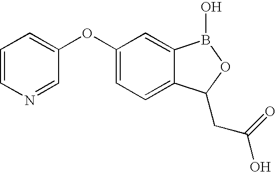

To a solution of 1-hydroxy-6-(pyridine-3-yoxyl)-1,3-dihydro-benzo[c][1,2]oxaborol-3-yl]-acetic acid ethyl ester (0.104 g, 0.33 mmol) in MeOH (4 mL) and H₂O (4 mL) was added LiOH (0.040 g) at 0° C. The resulting mixture was stirred at room temperature for 24 hours then cooled to 0° C. The reaction mixture was acidified to pH 3 using 6M HCl then concentrated in vacuo. The residue was purified by silica gel flash column chromatography to give (1-hydroxy-6-pyridin-3-yoxyl)-1,3-dihydro-benzo[c][1,2]oxaborol-3-yl)-acetic acid (0.040 g, 43%). ¹H NMR (400 MHz, DMSO-d₆) δ 9.24 (br s, 1H), 8.43 (m, 2H), 7.50 (m, 3H), 7.25 (m, 2H), 5.44 (m, 1H), 3.00-2.90 (m, 1H), 2.40-2.30 (m, 1H). MS (ESI) m/z=286 [M+H]⁺, HPLC purity: 97.97% (220 nm), 97.72% (Maxplot).

### E73 [1-Hydroxy-6-(pyridine-3-yoxyl)-1,3-dihydro-benzo[c][1,2]oxaborol-3-yl]-acetic acid ethyl ester

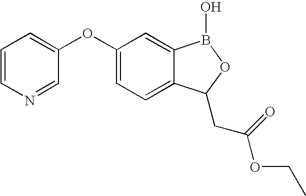

To a suspension of zinc dust (1.46 g, 22.5 mmol) in THF (10 mL) was added trimethylsilyl chloride (0.28 mL, 2.25 mmol) at 40° C. The mixture was heated to 55° C. and stirred for 15 minutes. After cooling down to 37° C., ethyl bromoacetate (2.16 mL, 19.5 mmol) was slowly added to the reaction mixture at 37-40° C. After addition, the resulting mixture was allowed to cool to room temperature over 30 minutes. This solution was added to a solution of 4-(pyridine-3-yloxy)-2-(4,4,5,5)tetramethyl-[1,3,2]dioxaborolan-2-yl)benzaldehyde (0.49 g, 1.5 mmol) in THF (6 mL) at 0° C. The mixture was stirred for 10 minutes before treating with saturated NH₄Cl (10 mL) and extracted with EtOAc (2×25 mL). The organic extracts were washed with brine, dried and concentrated in vacuo. The residue was diluted with H₂O and lyophilized to give [1-hydroxy-6-(pyridine-3-yoxyl)-1,3-dihydro-benzo[c][1,2]oxaborol-3-yl]-acetic acid ethyl ester (0.480 g, 100%). ¹H NMR (400 MHz, DMSO-d₆) δ 9.22 (s, 1H), 8.40 (m, 1H), 7.50 (d, J=6.5 Hz, 1H), 7.43 (m, 2H), 7.21 (m, 2H), 5.44 (m, 1H), 4.10 (m, 2H), 3.20-3.00 (m, 1H), 2.50-2.40 (m, 1H), 1.20 (m, 3H). MS (ES) m/z: 314 (M+1)⁺; HPLC purity: 99.01% (220 nm), 92.03% (254 nm), 98.62% (Maxplot).

### E74 [6-(2-Chloro-pyridin-4-yloxy)-1-hydroxy-1,3-dihydro-benzo[c][1,2]oxaborol-3-yl]-acetic acid

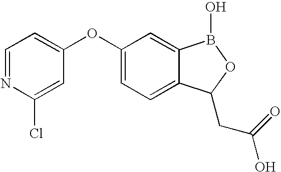

### Step 1: [6-(2-Chloro-pyridin-4-yloxy)-1-hydroxy-1,3-dihydro-benzo[c][1,2]oxaborol-3-yl]-acetic acid ethyl ester

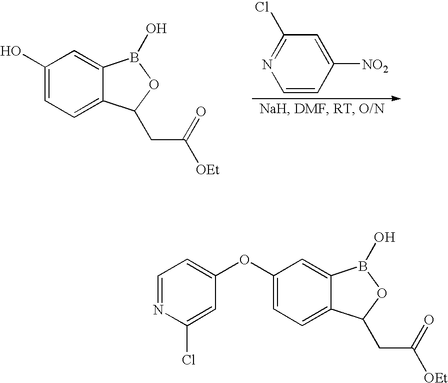

To a solution of (1,6-dihydroxy-1,3-dihydro-benzo[c][1,2]oxaborol-3-yl)-acetic acid ethyl ester (0.40 g, 1.86 mmol)in DMF (4 mL) was added NaH (0.22 g, 5.59 mmol). The mixture was stirred at room temperature for 10 minutes. 2-Chloro-4-nitro-pyridine (0.74 g, 4.65 mmol) was added and the mixture stirred at room temperature for 16 hours. The reaction mixture was acidified with HCl and concentrated in vacuo. The residue was purified by silica gel flash column chromatography to give [6-(2-chloro-pyridin-4-yloxy)-1-hydroxy-1,3-dihydro-benzo[c][1,2]oxaborol-3-yl]-acetic acid ethyl ester (0.500 g, 77%). ¹H NMR (400 MHz, DMSO-d6) δ 9.40 (s, 1H), 8.60 (d, 1H), 7.50 (s, 1H), 7.37 (d, 1H), 7.00 (m, 1H), 6.97 (m, 1H), 5.50 (m, 1H), 4.10 (m, 2H), 3.10 (m, 1H), 2.40 (m, 1H), 1.20 (m, 3H).

### Step 2: [6-(2-Chloro-pyridin-4-yloxy)-1-hydroxy-1,3-dihydro-benzo[c][1,2]oxaborol-3-yl]-acetic acid

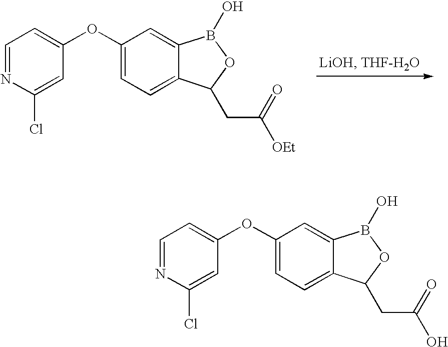

To a solution of [6-(2-chloro-pyridin-4-yloxy)-1-hydroxy-1,3-dihydro-benzo[c][1,2]oxaborol-3-yl]-acetic acid ethyl ester (0.660 g, 1.90 mmol) in THF (20 mL) and water (10 mL) was added LiOH (0.450 g) at 0° C. The resulting mixture was stirred at room temperature for 2 hours then cooled to 0° C. and acidified to pH 3 with 6N HCl. The mixture was concentrated in vacuo and the residue purified by preparative HPLC to give [6-(2-chloro-pyridin-4-yloxy)-1-hydroxy-1,3-dihydro-benzo[c][1,2]oxaborol-3-yl]-acetic acid (0.520 g, 90%). ¹H NMR (400 MHz, DMSO-d6) δ 12.40 (br s, 1H), 9.34 (s, 1H), 8.31 (d, J=3.2 Hz, 1H), 7.59 (d, J=8.4 Hz, 1H), 7.44 (s, 1H), 7.36 (d, J=2.4 Hz, 1H), 7.03 (s, 1H), 6.96 (d, J=2.4 Hz, 1H), 5.49 (m, 1H), 3.01 (m, 1H), 2.41 (m, 1H). MS (ESI) m/z=320 [M+H]+. HPLC: 98.71% (220 nm); 98.44% (Maxplot).

### E75 [6-(6-Fluoro-pyridin-2-yloxy)-1-hydroxy-1,3-dihydro-benzo[c][1,2]oxaborol-3-yl)-acetic acid

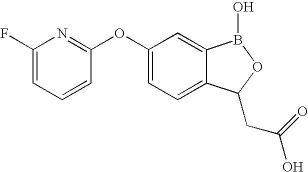

### Step 1: [6-(6-Fluoro-pyridin-2-yloxy)-1-hydroxy-1,3-dihydro-benzo[c][1,2]oxaborol-3-yl)-acetic acid

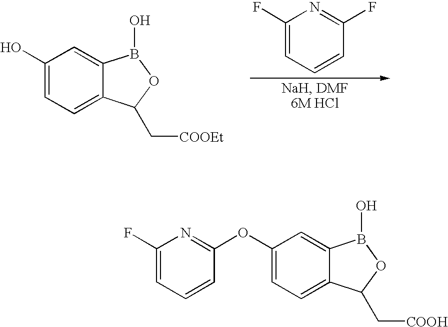

To a solution of (1,6-dihydroxy-1,3-dihydro-benzo[c][1,2]oxaborol-3-yl)-acetic acid ethyl ester (0.1 g, 0.42 mmol) in anhydrous DMF (2 mL) was added sodium hydride (0.043 g, 1.05 mmol) at 0° C. followed by 2,6-difluoro-pyridine (0.122 g, 1.05 mmol). The resulting mixture was stirred at room temperature for 18 hours then quenched with crushed ice. The pH was adjusted to 4 with 6M HCl and the mixture extracted with EtOAc. The organic extracts were washed with water, brine, dried over Na₂SO₄, filtered, and concentrated in vacuo. The residue was purified by preparative HPLC to give [6-(6-fluoro-pyridin-2-yloxy)-1-hydroxy-1,3-dihydro-benzo[c][1,2]oxaborol-3-yl)-acetic acid as an off white solid after lyophilization (0.027 g). mp 138.6-140° C. ¹H NMR 400 MHz (DMSO-d₆) δ 12.40 (s, 1H), 9.27 (s, 1H), 8.04 (q, J=8.4 Hz, 1H), 7.54 (d, J=8.4 Hz, 1H), 7.41 (s, 1H), 7.30 (d, J=10.4 Hz, 1H), 6.96 (d, J=8.4 Hz, 1H), 6.88 (d, J=8.0 Hz, 1H), 5.48 (m, 1H), 2.99 (dd, J=15.6, 4.0 Hz, 1H), 2.38 (m, 1H). MS (ESI) m/z: 302 (M−1)⁻. HPLC purity: 98.58% (Maxplot), 99.2% (220 nm).

### E76 [1-Hydroxy-6-(pyrimidin-4-yloxy)-1,3-dihydro-benzo[c][1,2]oxaborol-3-yl]-acetic acid

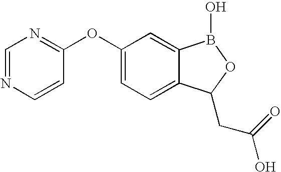

### Step 1: [1-Hydroxy-6-(pyrimidin-4-yloxy)-1,3-dihydro-benzo[c][1,2]oxaborol-3-yl]-acetic acid ethyl ester

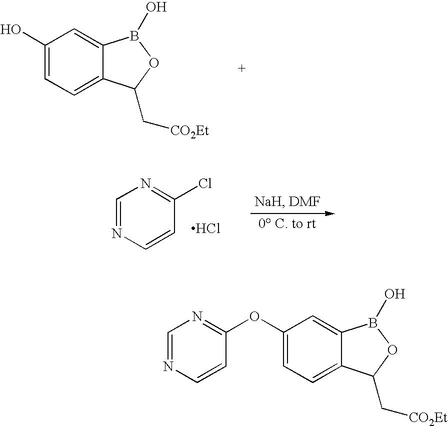

To a solution of (1,6-dihydroxy-1,3-dihydro-benzo[c][1,2]oxaborol-3-yl)-acetic acid ethyl ester (0.4 g, 1.69 mmol) and 4-chloro-pyrimidine hydrochloride (0.51 g, 3.38 mmol) in DMF (10 mL) at 0° C. was added NaH (0.25 g, 5.08 mmol) in portions. The solution was allowed to warm to room temperature and stirred for 10 hours. Saturated NH₄Cl (10 mL) was added at 0° C. The mixture was acidified to pH˜3 with 1N HCl and extracted with EtOAc (2×10 mL). The organic extracts were washed with water (10 mL), dried and concentrated in vacuo. The residue was purified by silica gel flash column chromatography (DCM:MeOH 95:5) to give [1-hydroxy-6-(pyrimidin-4-yloxy)-1,3-dihydro-benzo[c][1,2]oxaborol-3-yl]-acetic acid ethyl ester (0.2 g, 37%). ¹H NMR (400 MHz, CDCl₃): δ 8.79 (s, 1H), 8.59 (d, J=5.6 Hz, 1H), 7.45-7.38 (m, 2H), 7.27-7.23 (m, 1H), 6.94 (d, J=6.0 Hz, 1H), 5.68-5.64 (m, 1H), 4.22 (q, J=7.2 Hz, 2H), 2.86 (dd, J=6.4, 16.8 Hz, 1H), 2.72-2.66 (m, 1H), 1.29 (t, J=7.6 Hz, 3H). MS (ESI) m/z=315 [M+H]⁺.

### Step 2: [1-Hydroxy-6-(pyrimidin-4-yloxy)-1,3-dihydro-benzo[c][1,2]oxaborol-3-yl]-acetic acid

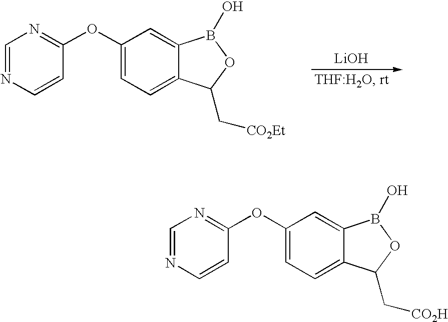

To a solution of [1-hydroxy-6-(pyrimidin-4-yloxy)-1,3-dihydro-benzo[c][1,2]oxaborol-3-yl]-acetic acid ethyl ester (0.37 g, 1.18 mmol) in THF:H₂O (1:1, 6 mL) at 0° C. was added a solution of LiOH (0.085 g, 3.53 mmol) in water (1 mL) The solution was allowed to warm to room temperature over 3 hours then acidified to pH 2 with 1N HCl at 0° C. The mixture was extracted with EtOAc (2×10 mL) and the organic extracts washed with water, dried and concentrated in vacuo. The residue was purified by preparative HPLC to give [1-hydroxy-6-(pyrimidin-4-yloxy)-1,3-dihydro-benzo[c][1,2]oxaborol-3-yl]-acetic acid (0.15 g, 45%). ¹H NMR (400 MHz, DMSO): δ 9.39 (s, 1H), 8.78 (d, J=6 Hz, 1H), 7.60 (d, J=8.4 Hz, 1H), 7.51 (s, 1H), 7.39 (d, J=6.4 Hz, 1H), 7.22 (d, J=5.2 Hz, 1H), 5.55 (d, J=6 Hz, 1H), 3.04 (d, J=15.6 Hz, 1H), 2.47-2.41 (m, 1H). MS (ESI) m/z=287 [M+H]+.

### E77 [6-(2-Benzylamino-pyrimidin-4-yloxy)-1-hydroxy-1,3-dihydro-benzo[c][1,2]oxaborol-3-yl]-acetic acid

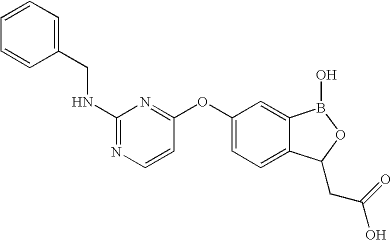

### Step 1: [6-(2-Chloro-pyrimidin-4-yloxy)-1-hydroxy-1,3-dihydro-benzo[c][1,2]oxaborol-3-yl]-acetic acid ethyl ester

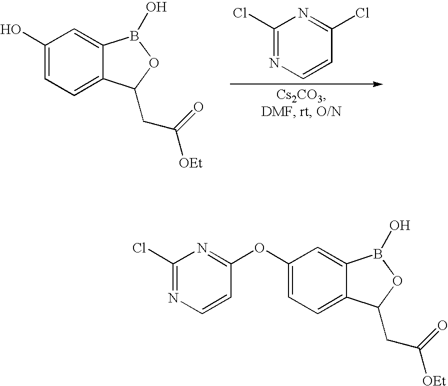

A solution of (1,6-dihydroxy-1,3-dihydro-benzo[c][1,2]oxaborol-3-yl)-acetic acid ethyl ester (0.10 g, 0.42 mmol) in DMF (2 mL) was treated with cesium carbonate (0.414 g, 1.27 mmol) at 0° C. followed by 2,4-dichloropyrimidine (0.094 g, 0.63 mmol). The mixture was stirred at room temperature for 18 hours. The mixture was quenched with water and extracted with ethyl acetate. The extracts were dried (Na₂SO₄), filtered and concentrated in vacuo. The residue was purified by silica gel flash column chromatography (EtOAc/hexane; 1:3 gradient) to give [6-(2-chloro-pyrimidin-4-yloxy)-1-hydroxy-1,3-dihydro-benzo[c][1,2]oxaborol-3-yl]-acetic acid ethyl ester as a light yellow oil (0.08 g, 52%). ¹H NMR (400 MHz, CD₃OD) δ 8.50 (d, J=5.86 Hz, 1H), 7.51 (d, J=8.21 Hz, 1H), 7.41 (s, 1H), 7.30 (d, J=8.21 Hz, 1H), 7.01 (d, J=5.86 Hz, 1H), 5.66-5.63 (m, 1H), 4.10 (q, J=7.03 Hz, 2H), 3.02 (dd, J=15.63, 4.30 Hz, 1H), 2.63 (dd, J=15.63, 9.18 Hz, 1H), 1.24 (t, J=7.03 Hz, 3H).

### Step 2: [6-(2-Benzylamino-pyrimidin-4-yloxy)-1-hydroxy-1,3-dihydro-benzo[c][1,2]oxaborol-3-yl]-acetic acid ethyl ester

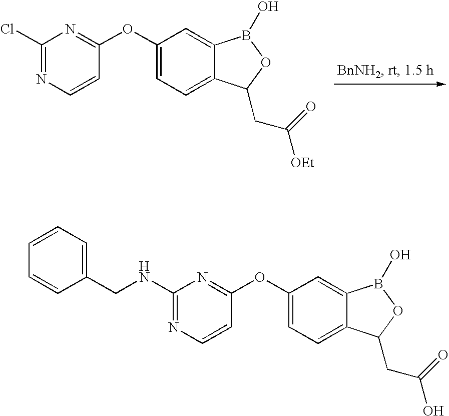

A solution of [6-(2-chloro-pyrimidin-4-yloxy)-1-hydroxy-1,3-dihydro-benzo[c][1,2]oxaborol-3-yl]-acetic acid ethyl ester (0.30 g, 086 mmol) and benzylamine (0.38 mL, 3.47 mmol) was stirred at room temperature for 1.5 hours. The solution was concentrated and residue was purified by silica gel flash column chromatography (MeOH:CH₂Cl₂; 1:99 gradient) to give [6-(2-benzylamino-pyrimidin-4-yloxy)-1-hydroxy-1,3-dihydro-benzo[c][1,2]oxaborol-3-yl]-acetic acid ethyl ester as a yellow foam (0.25 g, 69%). ¹H NMR (400 MHz, CD₃OD) δ 8.06 (d, J=5.85 Hz, 1H), 7.40-7.00 (m, 8H), 6.15 (d, J=5.46 Hz, 1H), 5.65 (dd, J=8.20, 2.34 Hz, 1H), 4.40-4.20 (m, 2H), 4.18-4.03 (m, 2H), 2.90 (dd, J=15.22, 3.12 Hz, 1H), 2.60 (dd, J=15.22, 8.98 Hz, 1H), 1.20 (t, J=7.03 Hz, 3H).

### Step 3: [6-(2-Benzylamino-pyrimidin-4-yloxy)-1-hydroxy-1,3-dihydro-benzo[c][1,2]oxaborol-3-yl]-acetic acid

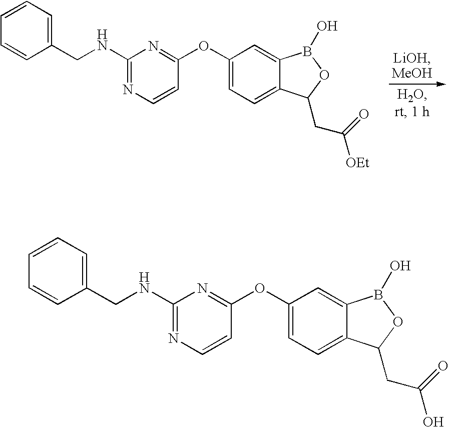

A solution of [6-(2-benzylamino-pyrimidin-4-yloxy)-1-hydroxy-1,3-dihydro-benzo[c][1,2]oxaborol-3-yl]-acetic acid ethyl ester (0.25 g, 0.60 mmol) in methanol (2 mL) was treated with lithium hydroxide (0.071 g, 2.96 mmol) in water (2 mL) at room temperature. The solution was stirred at room temperature for 1 hour then quenched with 2N HCl to pH 2. The precipitated solid was collected by vacuum filtration to give [6-(2-benzylamino-pyrimidin-4-yloxy)-1-hydroxy-1,3-dihydro-benzo[c][1,2]oxaborol-3-yl]-acetic acid as a white solid (0.11 g, 47%). ¹H NMR (400 MHz, DMSO-d6) δ 9.20 (br s, 1H), 8.40-8.10 (br m, 1H), 7.60 (d, J=7.81 Hz, 1H), 7.51 (s, 1H), 7.42-7.10 (m, 5H), 6.89 (br s, 1H), 6.70-6.40 (br m, 1H), 5.60-5.45 (m, 1H), 4.48 (br s, 1H), 4.19 (s, 2H), 3.02 (d, J=15.24 Hz,1H), 2.36 (dd, J=15.24, 9.19 Hz, 1H). MS (ESI) m/z: 392[M+1].

### E78 [1-Hydroxy-6-(pyrimidin-2-yloxy)-1,3-dihydro-benzo[c][1,2]oxaborol-3-yl]-acetic acid

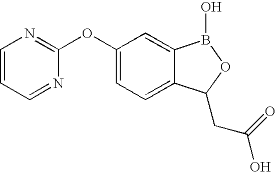

### Step 1: 2-Hydroxy-4-(tetrahydro-pyran-2-yloxy)-benzaldehyde

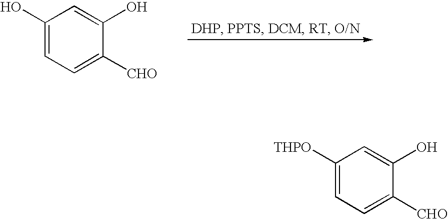

To a mixture of 2,4-dihydroxy-benzaldehyde (6.9 g, 50 mmol) in dichloromethane (50 mL) was added 3,4-dihydro-2H-pyran (6.8 mL, 75 mmol) and pyridium p-toluenesulfonic acid (0.050 g) at room temperature. The resulting mixture was stirred at room temperature for 18 hours then concentrated in vacuo. The residue was purified by silica gel flash column chromatography to give 2-hydroxy-4-(tetrahydro-pyran-2-yloxy)-benzaldehyde (7.04 g, 62%). ¹HNMR (400 MHz, CDCl₃): 11.40 (s, 1H), 9.88 (s, 1H), 7.43 (d, J=8.1 Hz, 1H), 6.65 (m, 2H), 5.51 (m, 1H), 3.80 (m, 1H), 3.65 (m, 1H), 2.00-1.50 (m, 6H).

### Step 2: Trifluoro-methanesulfonic acid 2-formyl-5-(tetrahydro-pyran-2-yloxy)-phenyl ester

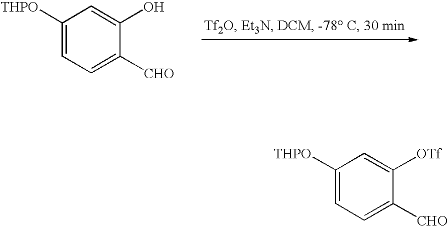

To a solution of 2-hydroxy-4-(tetrahydro-pyran-2-yloxy)-benzaldehyde (2.08 g, 9.37 mmol) and Et₃N (3.91 mL, 28.11 mmol) in dichloromethane (20 mL) was slowly added Tf₂O (1.42 mL, 11.24 mmol) at −78° C. The mixture was stirred at −78° C. for 30 minutes. The mixture was diluted with cold brine and extracted with dichloromethane. The organic extracts were washed with brine, dried and concentrated in vacuo. The residue was dissolved in Hexane-EtOAc(4:1) and passed through a plug of silica gel and concentrated in vacuo to give trifluoro-methanesulfonic acid 2-formyl-5-(tetrahydro-pyran-2-yloxy)-phenyl ester (3.25 g, quant.). 1HNMR (400 MHz, CDCl₃): 10.10 (s, 1H), 7.93 (d, J=8.1 Hz, 1H), 7.20 (d, J=8.1 Hz, 1H), 7.07 (s, 1H), 5.60 (m, 1H), 3.80 (m, 1H), 3.65 (m, 1H), 2.00-1.50 (m, 6H).

### Step 3: 4-(Tetrahydro-pyran-2-yloxy)-2-(4,4,5,5-tetramethyl-[1,3,2]dioxaborolan-2-yl)-benzaldehyde

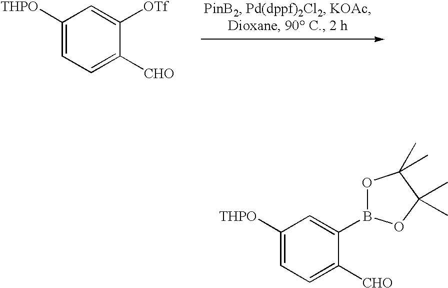

A solution of trifluoro-methanesulfonic acid 2-formyl-5-(tetrahydro-pyran-2-yloxy)-phenyl ester (3.25 g, 10.16 mmol), bis(pinacolato)diborane (3.35 g, 13.21 mmol) PdCl₂(dppf) (1.48 g, 2.03 mmol) and KOAc (2.99 g, 30.48 mmol) in dioxane (40 mL) was degassing for 10 minutes with bubbling N2. The reaction mixture was heated at 90° C. for 2 hours then diluted with EtOAc (100 mL). The mixture was filtered through a pad of celite and concentrated in vacuo. The residue was purified by silica gel flash column chromatography to give 4-(tetrahydro-pyran-2-yloxy)-2-(4,4,5,5-tetramethyl-[1,3,2]dioxaborolan-2-yl)-benzaldehyde as a yellow oil (5.2 g). 1HNMR (400 MHz, CDCl₃): 10.40 (s, 1H), 7.93 (d, J=8.1 Hz, 1H), 7.44 (s, 1H), 7.20 (m, 1H), 5.60 (m, 1H), 3.80 (m, 1H), 3.60 (m, 1H), 2.00-1.50 (m, 6H), 1.40 (s, 12H).

### Step 4: (1,6-Dihydroxy-1,3-dihydro-benzo[c][1,2]oxaborol-3-yl)-acetic acid ethyl ester

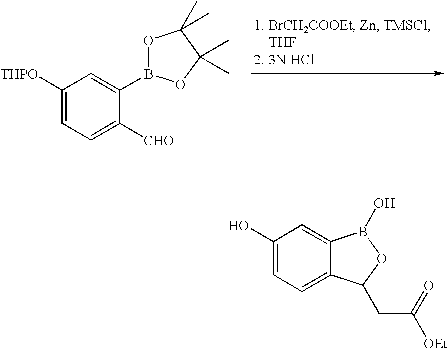

To a suspension of zinc dust (5.35 g, 82.3 mmol) in THF (10 mL) was added trimethylsilyl chloride (1.1 g, 10.15 mmol) at 40° C. The mixture was heated to 55° C. and stirred for 45 minutes. After cooling down to 37° C., ethyl bromoacetate (7.58 mL, 74.87 mmol) was slowly added to the reaction mixture at 37-40° C. After addition, the resulting mixture was allowed to cool to room temperature over 30 minutes. This solution was added to a solution of 4-(tetrahydro-pyran-2-yloxy)-2-(4,4,5,5-tetramethyl-[1,3,2]dioxaborolan-2-yl)-benzaldehyde (8.18 g, 29 mmol) in THF (6 mL) at 0° C. The mixture was stirred for 10 minutes before treating with 3 N HCl and extracting with EtOAc (2×25 mL). The organic extracts were washed with brine, dried and concentrated in vacuo. The residue was diluted with water and lyophilized to give (1,6-dihydroxy-1,3-dihydro-benzo[c][1,2]oxaborol-3-yl)-acetic acid ethyl ester (4.1 g, 60%). ¹H NMR (400 MHz, DMSO-d6) δ 9.40 (s, 1H), 7.80 (d, J=8.4 Hz, 1H), 7.00 (s, 1H), 6.80 (d, J=8.4 Hz, 1H), 5.30 (m, 1H), 4.10 (m, 1H), 2.90 (m,1H), 2.30 (m, 1H), 1.20 (m, 3H).

Step 5: [1-Hydroxy-6-(pyrimidin-2-yloxy)-1,3-dihydro-benzo[c][1,2]oxaborol-3-yl]-acetic acid ethyl ester

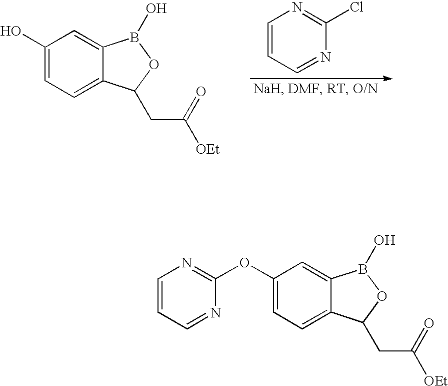

To a solution of (1,6-dihydroxy-1,3-dihydro-benzo[c][1,2]oxaborol-3-yl)-acetic acid ethyl ester (0.33 g, 1.40 mmol) in DMF (4 mL) was added NaH (0.17 g, 4.20 mmol). The mixture was stirred at room temperature for 10 minutes. 2-Chloropyrimidine (0.40 g, 3.50 mmol) was added and the mixture stirred at room temperature for 48 hours. The reaction mixture was acidified with HCl and concentrated in vacuo. The residue was purified by silica gel flash column chromatography to give a mixture of [1-hydroxy-6-(pyrimidin-2-yloxy)-1,3-dihydro-benzo[c][1,2]oxaborol-3-yl]-acetic acid ethyl ester and [1-hydroxy-6-(pyrimidin-2-yloxy)-1,3-dihydro-benzo[c][1,2]oxaborol-3-yl]-acetic acid. (0.280 g, 64%).

### Step 6: [1-Hydroxy-6-(pyrimidin-2-yloxy)-1,3-dihydro-benzo[c][1,2]oxaborol-3-yl]-acetic acid

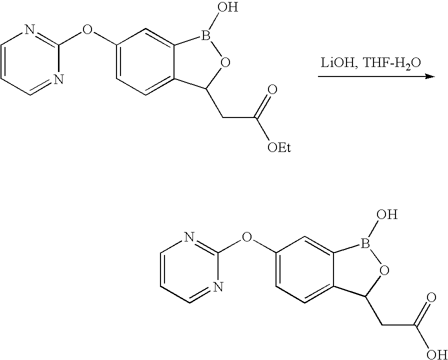

To a solution of [1-hydroxy-6methoxy-1,3-dihydro-benzo[c][1,2]oxaborol-3-yl]-acetic acid ethyl ester (0.28 g, 0.89 mmol) in THF (8 mL) and water (4 mL) was added LiOH (0.220 g) at 0° C. The resulting mixture was stirred at room temperature for 2 hours then cooled to 0° C. and acidified to pH 3 with 6N HCl. The mixture was concentrated in vacuo and the residue purified by preparative HPLC to give [1-hydroxy-6-(pyrimidin-2-yloxy)-1,3-dihydro-benzo[c][1,2]oxaborol-3-yl]-acetic acid (0.120 g, 47%). ¹H NMR (400 MHz, DMSO-d6) δ 9.20 (s, 1H), 8.60 (s, 2H), 7.20 (m, 2H), 7.15 (m, 2H), 5.70 (m, 1H), 3.20 (m, 1H), 2.44 (s, 1H).

### E79 3-(1-Hydroxy-6-pyridin-3-yoxyl)-1,3-dihydro-benzo[c][1,2]oxaborol-3-yl)-propionic acid

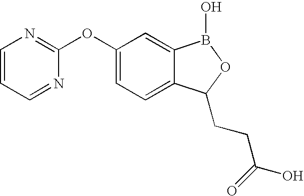

### Step 1: 4-(2,4-Dihydroxy-phenyl)-4-oxo-butyric acid

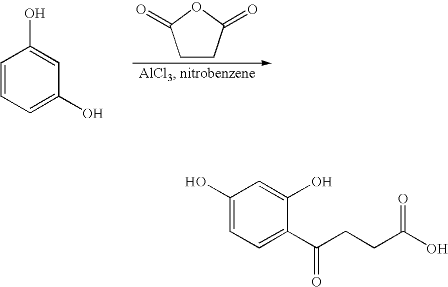

To a suspension of benzene-1,3-diol (11 g, 100 mmol) and succinic anhydride (11 g, 110 mmol) in nitrobenzene (100 mL) was added AlCl₃ (67 g, 500 mmol). The reaction mixture was heated at 90° C. for 3 hours then at 50° C. for 16 hours. After cooling to room temperature, the mixture was poured onto ice and acidified with HCl. The aqueous solution was washed with dichloromethane and concentrated to ˜100 mL volume. The solution was left to stand overnight and the precipitated solid was collected to give 4-(2,4-dihydroxy-phenyl)-4-oxo-butyric acid (8.6 g, 41%). 1HNMR (400MHz, DMSO-d6): 12.40 (s, 1H), 12.10 (br s, 1H), 10.62 (s, 1H), 7.80 (d, J=8.4 Hz, 1H), 6.40 (d, J=8.4 Hz, 1H), 6.29 (s, 1H), 3.20 (t, J=7.8 Hz, 2H), 2.50 (t, J=7.8 Hz, 2H).

### Step 2: 4-(2,4-Dihydroxy-phenyl)-4-oxo-butyric acid methyl ester

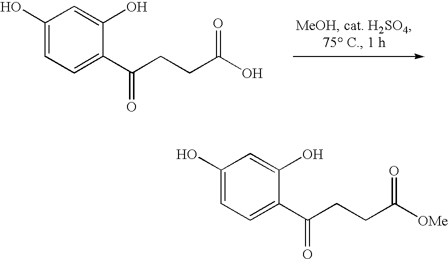

A mixture of 4-(2,4-dihydroxy-phenyl)-4-oxo-butyric acid (5.8 g, 27.62 mmol) and concentrated H2SO4 (0.5 mL) in MeOH was heated at 75° C. for 1 hour then concentrated in vacuo. The residue was dissolved in EtOAc, passed through a plug of silica gel and concentrated to give 4-(2,4-dihydroxy-phenyl)-4-oxo-butyric acid methyl ester (6.0 g, 100%). 1HNMR (400 MHz, CDCl₃): 12.40 (s, 1H), 7.81 (d, J=8.4 Hz, 1H), 6.40 (m, 2H), 5.90 (br s, 1H), 3.27 (t, J=7.8 Hz, 2H), 2.78 (t, J=7.8 Hz, 2H).

### Step 3: 4-[2-Hydroxy-4-(tetrahydro-pyran-2-yloxy)-phenyl]-4-oxo-butyric acid methyl ester

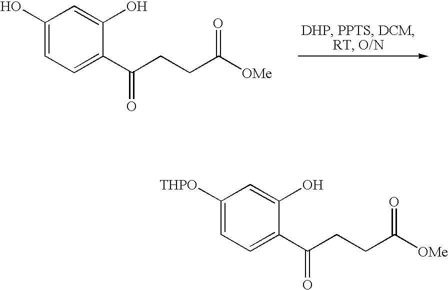

To a mixture of 4-(2,4-dihydroxy-phenyl)-4-oxo-butyric acid methyl ester (8.7 g, 38.84 mmol) in dichloromethane (50 mL) was added 3,4-dihydro-2H-pyran (7.7 mL, 85.44 mmol) and pyridium p-toluenesulfonic acid (0.050 g) at room temperature. The resulting mixture was stirred at room temperature for 16 hours then concentrated in vacuo. The residue was purified by silica gel flash column chromatography to give 4-[2-hydroxy-4-(tetrahydro-pyran-2-yloxy)-phenyl]-4-oxo-butyric acid methyl ester (10.96 g, 92%). 1HNMR (400 MHz, CDCl₃): 12.40 (s, 1H), 7.60 (d, J=8.4 Hz, 1H), 6.35 (m, 2H), 6.07 (s, 1H), 3.77 (s, 3H), 3.27 (t, J=8.4 Hz, 2H), 2.79 (t, J=8.4 Hz, 2H).

### Step 4: 4-Oxo-4-[4-(tetrahydro-pyran-2-yloxy)-2-trifluoromethanesulfonyloxy-phenyl]-butyric acid methyl ester

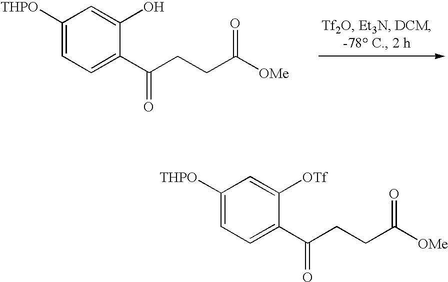

To a solution of 4-[2-hydroxy-4-(tetrahydro-pyran-2-yloxy)-phenyl]-4-oxo-butyric acid methyl ester (10.96 g, 35.58 mmol) and Et3N (14.85 mL, 107 mmol) in dichloromethane (100 mL) was slowly added Tf2O (9.60 mL, 56.94 mmol) at −78° C. The mixture was stirred at −78° C. for 2 hours. The mixture was diluted with water and extracted with dichloromethane. The organic extracts were washed with brine, dried and concentrated in vacuo. The residue was dissolved in Hexane-EtOAc(4:1), filtered through a plug of silica gel and the filtrate was concentrated to give 4-oxo-4-[4-(tetrahydro-pyran-2-yloxy)-2-trifluoromethanesulfonyloxy-phenyl]-butyric acid methyl ester (15.62 g, quant.). ¹H NMR (400MHz, CDCl₃): 7.80 (d, J=8.4 Hz, 1H), 7.10 (d, J=8.4 Hz), 7.00 (s, 1H), 5.50 (m, 1H), 3.80 (m, 1H), 3.70 (s, 3H), 3.60 (m, 1H), 3.22 (t, J=8.8 Hz, 2H), 2.79 (t, J=8.8 Hz, 2H), 2.00-1.50 (m, 6H).

### Step 5: 4-tho-4-[4-(tetrahydro-pyran-2-yloxy)-2-(4,4,5,5-tetramethyl-[1,3,2]dioxaborolan-2-yl)-phenyl]-butyric acid methyl ester

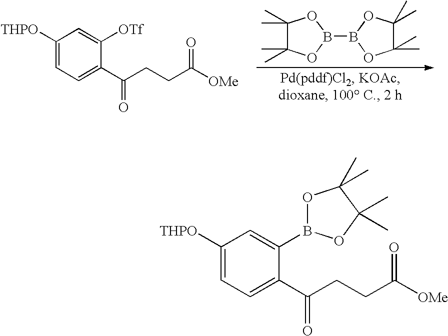

A solution of 4-oxo-4-[4-(tetrahydro-pyran-2-yloxy)-2-trifluoromethanesulfonyloxy-phenyl]-butyric acid methyl ester (14.75 g, 33.52 mmol), bis(pinacolato)diborane (17.03 g, 67.05 mmol), PdCl₂(dppf) (2.45 g, 3.35 mmol) and KOAc (9.85 g, 101 mmol) in dioxane (150 mL) was degassed for 10 minutes with bubbling N2. The reaction mixture was heated at 100° C. for 2 hours then diluted with EtOAc (100 mL). The mixture was filtered through a pad of celite and filtrate was concentrated in vacuo. The residue was purified by silica gel flash column chromatography to give 4-oxo-4-[4-(tetrahydro-pyran-2-yloxy)-2-(4,4,5,5-tetramethyl-[1,3,2]dioxaborolan-2-yl)-phenyl]-butyric acid methyl ester as a yellow oil (11.84 g, 84%). 1HNMR (400 MHz, CDCl₃): 7.80 (d, J=8.4 Hz, 1H), 7.10 (s, 1H), 7.05 (d, J=8.4 Hz, 1H), 5.59 (m, 1H), 3.80 (m, 1H), 3.70 (s, 3H), 3.60 (m, 1H), 3.27 (t, J=8.8 Hz, 2H), 2.78 (t, J=8.8 Hz, 2H), 2.00-1.50 (m, 6H), 1.40 (s, 12H).

### Step 6: 3-(1,6-Dihydroxy-1,3-dihydro-benzo[c][1,2]oxaborol-3-yl)-propionic acid methyl ester

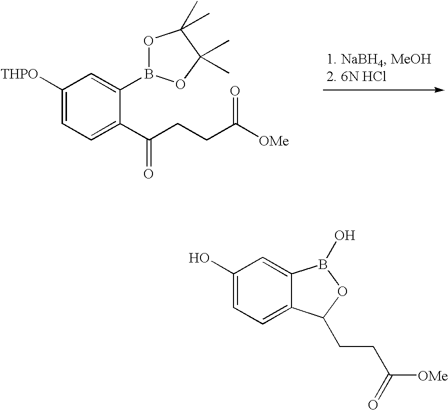

To a solution of 4-oxo-4-[4-(tetrahydro-pyran-2-yloxy)-2-(4,4,5,5-tetramethyl-[1,3,2]dioxaborolan-2-yl)-phenyl]-butyric acid methyl ester (11.83 g, 28.30 mmol) in MeOH (50 mL) was added NaBH₄ (2.36 g, 62.26 mmol) at 0° C. The reaction mixture was stirred at 0° C. for 30 minutes, quenched with 6 N HCl and concentrated in vacuo. The residue was purified by silica gel flash column chromatography and lyophilized to give 3-(1,6-dihydroxy-1,3-dihydro-benzo[c][1,2]oxaborol-3-yl)-propionic acid methyl ester (4.2 g, 62%). ¹H NMR (400 MHz, DMSO-d6) δ 9.35 (s, 1H), 9.00 (s, 1H), 7.20 (d, J=8.4 Hz, 1H), 7.03 (s, 1H), 6.84 (d, J=8.4 Hz, 1H), 5.00 (m, 1H), 3.60 (s, 3H), 2.40-2.10 (m, 3H), 1.65 (m, 1H).

### Step 7: 3-[1-Hydroxy-6-(pyrimidin-2-yloxy)-1,3-dihydro-benzo[c][1,2]oxaborol-3-yl]-propionic acid methyl ester

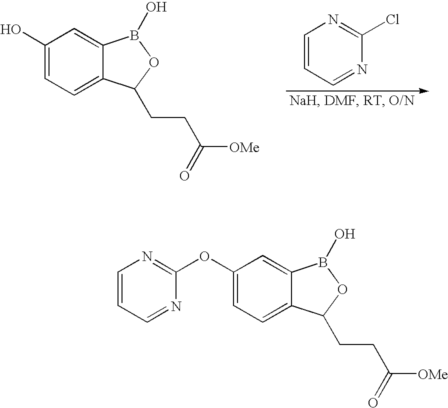

To a solution of 3-(1,6-dihydroxy-1,3-dihydro-benzo[c][1,2]oxaborol-3-yl)-propionic acid methyl ester (0.36 g, 1.50 mmol) in DMF (5 mL) was added NaH (0.20 g, 4.50 mmol). The mixture was stirred at room temperature for 10 minutes. 2-Chloropyrimidine (0.43 g, 3.75 mmol) was added and the mixture stirred at room temperature for 48 hours. The reaction mixture was acidified with HCl and concentrated in vacuo. The residue was purified by silica gel flash column chromatography to give 3-[1-hydroxy-6-(pyrimidin-2-yloxy)-1,3-dihydro-benzo[c][1,2]oxaborol-3-yl]-propionic acid methyl ester (0.260 g, 55%). ¹H NMR (400 MHz, MeOD-d4) δ 8.60 (m, 2H), 7.50-7.20 (m, 4H), 5.30 (m, 1H), 3.66 (s, 3H), 2.46 (m, 3H), 1.86 (m, 1H).

### Step 8: 3-[1-Hydroxy-6-(pyrimidin-2-yloxy)-1,3-dihydro-benzo[c][1,2]oxaborol-3-yl]-propionic acid

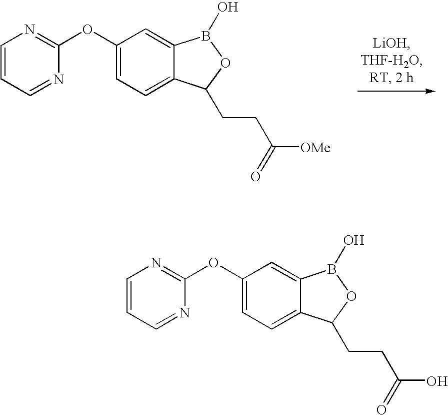

To a solution of 3-[1-hydroxy-6-(pyrimidin-2-yloxy)-1,3-dihydro-benzo[c][1,2]oxaborol-3-yl]-propionic acid methyl ester (0.26 g, 0.83 mmol) in THF (8 mL) and water (2 mL) was added LiOH (0.200 g) at 0° C. The resulting mixture was stirred at room temperature for 2 hours then cooled to 0° C. and acidified to pH 3 with 6N HCl. The mixture was concentrated in vacuo and the residue purified by preparative HPLC to give 3-[1-hydroxy-6-(pyrimidin-2-yloxy)-1,3-dihydro-benzo[c][1,2]oxaborol-3-yl]-propionic acid (0.100 g, 47%). ¹H NMR (400 MHz, DMSO-d6) δ 9.23 (br s, 1H), 8.65 (m, 2H), 7.44 (m, 2H), 7.30 (m, 2H), 5.17 (m, 1H), 2.40-2.20 (m, 3H), 1.70 (m, 1H). MS (ES) m/z: 299 (M−1)−. HPLC purity: 97.18% (220 nm), 98.12% (Maxplot).

### E80 [1-Hydroxy-6-(pyrazin-2-yloxy)-1,3-dihydro-benzo[c][1,2]oxaborol-3-yl]-acetic acid

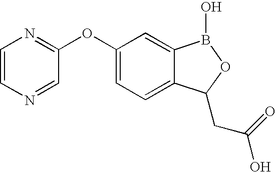

### Step 1: [1-Hydroxy-6-(pyrazin-2-yloxy)-1,3-dihydro-benzo[c][1,2]oxaborol-3-yl]-acetic acid ethyl ester

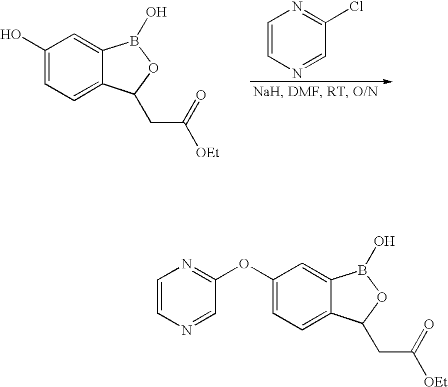

To a solution of (1,6-dihydroxy-1,3-dihydro-benzo[c][1,2]oxaborol-3-yl)-acetic acid ethyl ester (0.33 g, 1.40 mmol) in DMF (4 mL) was added NaH (0.17 g, 4.20 mmol). The mixture was stirred at room temperature for 10 minutes. Chloropyrazine (0.40 g, 3.50 mmol) was added and the mixture stirred at room temperature for 48 hours. The reaction mixture was acidified with HCl and concentrated in vacuo. The residue was purified by silica gel flash column chromatography to give a mixture of [1-hydroxy-6-(pyrazin-2-yloxy)-1,3-dihydro-benzo[c][1,2]oxaborol-3-yl]-acetic acid ethyl ester and [1-hydroxy-6-(pyrazin-2-yloxy)-1,3-dihydro-benzo[c][1,2]oxaborol-3-yl]-acetic acid ethyl ester (0.240 g, 55%).

### Step 2: [1-Hydroxy-6-(pyrazin-2-yloxy)-1,3-dihydro-benzo[c][1,2]oxaborol-3-yl]-acetic acid

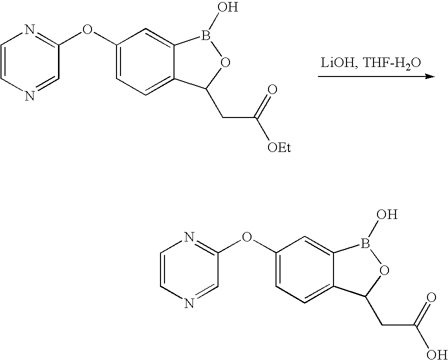

To a solution of [1-hydroxy-6-methoxy-1,3-dihydro-benzo[c][1,2]oxaborol-3-yl]-acetic acid ethyl ester (0.240 g, 0.76 mmol) in THF (8 mL) and water (4 mL) was added LiOH (0.220 g) at 0° C. The resulting mixture was stirred at room temperature for 2 hours then cooled to 0° C. and acidified to pH 3 with 6N HCl. The mixture was concentrated in vacuo and the residue purified by preparative HPLC to give [1-hydroxy-6-(pyrazin-2-yloxy)-1,3-dihydro-benzo[c][1,2]oxaborol-3-yl]-acetic acid (0.100 g, 46%). ¹H NMR (400 MHz, DMSO-d6) δ 12.39 (s, 1H), 9.20(s, 1H), 8.56 (s, 1H), 8.38 (s, 1H), 8.22 (s, 1H), 7.27 (s, 1H), 7.16 (s, 1H), 5.56 (s, 1H), 3.05 (m, 1H), 2.65 (m, 2H), 2.19 (M, 1H).

### E81 [1-Hydroxy-6-([1,3,4]thiadiazol-2-yloxy)-1,3-dihydro-benzo[c][1,2]oxaborol-3-yl]-acetic acid

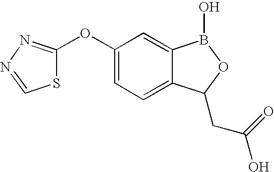

### Step 1: 2-Bromo-5-nitro-[1,3,4]thiadiazole

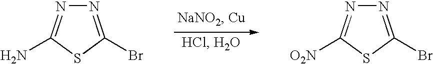

To a mixture of Cu (1.0 g, 15 mmol) and NaNO₂ (1.0 g, 15 mmol) in water (10 mL) was added 2 drops of concentrated HCl at room temperature and stirred for 15 minutes. A warm solution of 5-bromo-[1,3,4]thiadiazol-2-ylamine (0.9 g, 5 mmol) in aqueous HCl (4M, 10 mL) was added over a period of 15 minutes. The resulting mixture was stirred for 2 hours and the precipitated yellow solid was filtered and washed with water (20 mL). The solid was dissolved in ether (25 mL), filtered and the filtrate concentrated in vacuo to give 2-bromo-5-nitro-[1,3,4]thiadiazole (0.25 g, 25%). MS (ESI) m/z=211 [M+H]+.

### Step 2: [6-(5-Bromo-[1,3,4]thiadiazol-2-yloxy)-1-hydroxy-4-methyl-1,3-dihydro-benzo[c][1,2]oxaborol-3-yl]-acetic acid ethyl ester and [1-Hydroxy-6-(5-nitro-[1,3,4]thiadiazol-2-yloxy)-1,3-dihydro-benzo[c][1,2]oxaborol-3-yl]-acetic acid ethyl ester

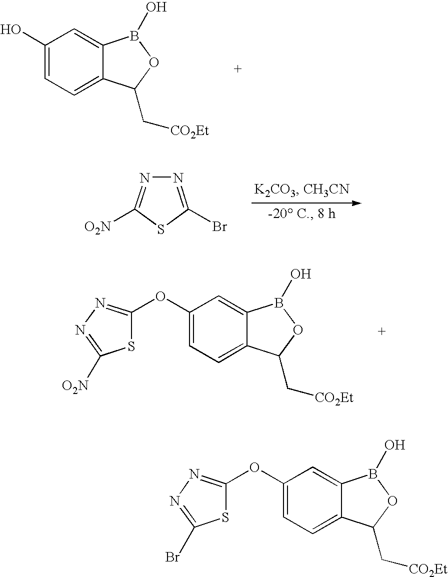

To a solution of (1,6-dihydroxy-1,3-dihydro-benzo[c][1,2]oxaborol-3-yl)-acetic acid ethyl ester (1.0 g, 4.23 mmol) and 2-bromo-5-nitro-[1,3,4]thiadiazole (1.78 g, 8.47 mmol) in CH₃CN (30 mL) at −20° C. was added K₂CO₃ (1.16 g, 8.47 mmol). The reaction mixture was stirred for 8 hours at −20° C. then concentrated in vacuo. The residue was dissolved in EtOAc (20 mL), washed with water (2×10 mL), dried and concentrated. The residue was purified by silica gel flash column chromatography to give 1.2 grams of a 3:1 mixture of products which was used without further purification. ¹H NMR (400 MHz, CDCl₃): δ 7.72 (d, J=2.4 Hz, 1H), 7.65-7.52 (m, 2H), 5.52-5.47 (m, 1H), 4.06 (q, J=7.2 Hz, 2H), 3.11-3.05 (m, 1H), 2.54-2.48 (m, 1H), 1.16 (t, J=8 Hz, 3H). MS (ESI) m/z=366 and 400 [M+H]+.

### Step 3: [1-Hydroxy-6-([1,3,4]thiadiazol-2-yloxy)-1,3-dihydro-benzo[c][1,2]oxaborol-3-y]-acetic acid ethyl ester and [6-(5-amino-[1,3,4]thiadiazol-2-yloxy)-1-hydroxy-1,3-dihydro-benzo[c][1,2]oxaborol-3-yl]-acetic acid ethyl ester

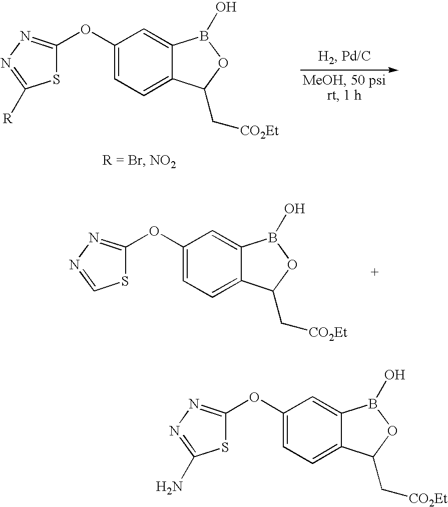

Pd/C (0.75 g) was added to a solution of [6-(5-bromo-[1,3,4]thiadiazol-2-yloxy)-1-hydroxy-4-methyl-1,3-dihydro-benzo[c][1,2]oxaborol-3-yl]-acetic acid ethyl ester and [1-hydroxy-6-(5-nitro-[1,3,4]thiadiazol-2-yloxy)-1,3-dihydro-benzo[c][1,2]oxaborol-3-yl]-acetic acid ethyl ester (3:1 mixture, 1.0 g) in MeOH (20 mL) and hydrogenated at 50 psi for 1 hour. The mixture was filtered through a pad of celite and concentrated in vacuo. The residue was purified by preparative HPLC to give 1-hydroxy-6-([1,3,4]thiadiazol-2-yloxy)-1,3-dihydro-benzo[c][1,2]oxaborol-3-yl]-acetic acid ethyl ester (0.070 g, 16%) and [6-(5-amino-[1,3,4]thiadiazol-2-yloxy)-1-hydroxy-1,3-dihydro-benzo[c][1,2]oxaborol-3-yl]-acetic acid ethyl ester (0.077 g, 20%).

1-hydroxy-6-([1,3,4]thiadiazol-2-yloxy)-1,3-dihydro-benzo[c][1,2]oxaborol-3-yl]-acetic acid ethyl ester. ¹H NMR (400 MHz, DMSO): δ 9.40 (s, 1H), 9.18 (s, 1H), 7.64-7.58 (m, 2H), 7.52 (dd, J=2.4, 8 Hz, 1H), 5.51 (dd, J=4, 9.2 Hz, 1H), 4.09 (q, J=6.8 Hz, 2H), 3.10 (dd, J=4, 15.6, 1H), 2.54-2.47 (m, 1H), 1.18 (t, J=6.8 Hz, 3H). MS (ESI) m/z=321 [M+H]+.

[6-(5-amino-[1,3,4]thiadiazol-2-yloxy)-1-hydroxy-1,3-dihydro-benzo[c][1,2]oxaborol-3-yl]-acetic acid ethyl ester. ¹H NMR (400 MHz, DMSO): δ 9.36 (s, 1H), 7.53-7.51 (m, 2H), 7.37 (dd, J=2.4 Hz, 8.4 Hz, 1H), 7.08 (s, 2H), 5.47 (dd, J=4, 8.8 Hz, 1H), 4.10 (q, J=6.8 Hz, 1H), 3.17-3.08 (m, 1H), 2.51-2.49 (m, 1H), 1.17 (t, J=6.8 Hz, 3H). MS (ESI) m/z=336 [M+H]+.

### Step 4: [1-Hydroxy-6-([1,3,4]thiadiazol-2-yloxy)-1,3-dihydro-benzo[c][1,2]oxaborol-3-yl]-acetic acid

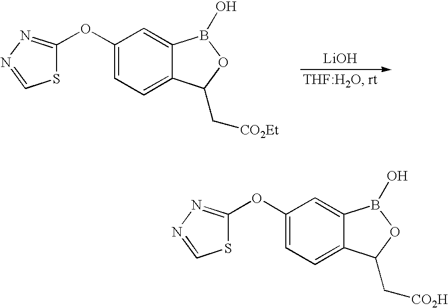

To a stirred solution of [1-hydroxy-6-([1,3,4]thiadiazol-2-yloxy)-1,3-dihydro-benzo[c][1,2]oxaborol-3-yl]-acetic acid ethyl ester (0.05 g, 0.15 mmol) in THF:H₂O (1:1, 5 mL) at 0° C. was added a solution of LiOH (0.01 g, 0.46 mmol) in water (1 mL). The solution was allowed to warm to room temperature over 3 hours then acidified to pH 2 with 1N HCl. The mixture was extracted with EtOAc (2×10 mL) and the organic extracts dried and concentrated in vacuo. The residue was purified by preparative HPLC to give [1-hydroxy-6-([1,3,4]thiadiazol-2-yloxy)-1,3-dihydro-benzo[c][1,2]oxaborol-3-yl]-acetic acid (0.04 g, 87%). ¹H NMR (400 MHz, DMSO): δ 9.15 (s, 1H), 7.66 (d, J=2.4 Hz, 1H), 7.60 (d, J=8.4 Hz, 1H), 7.50 (dd, J=2, 8.4 Hz, 1H), 5.50 (dd, J=4.4, 9.2 Hz, 1H), 3.0 (dd, J=3.2, 15.6 Hz, 1H), 2.44-2.37 (m, 1H). MS (ESI) m/z=291 [M−H]−.

### E82 [6-(5-Amino-[1,3,4]thiadiazol-2-yloxy)-1-hydroxy-1,3-dihydro-benzo[c][1,2]oxaborol-3-yl]-acetic acid

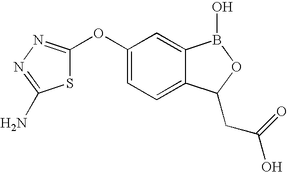

### Step 1: [6-(5-Amino-[1,3,4]thiadiazol-2-yloxy)-1-hydroxy-1,3-dihydro-benzo[c][1,2]oxaborol-3-yl]-acetic acid

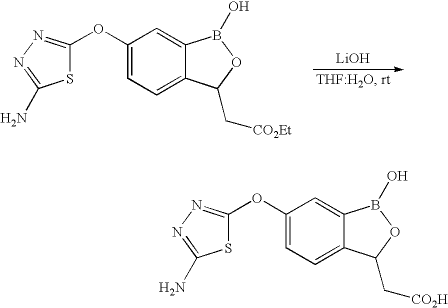

To a stirred solution of [6-(5-amino-[1,3,4]thiadiazol-2-yloxy)-1-hydroxy-1,3-dihydro-benzo[c][1,2]oxaborol-3-yl]-acetic acid ethyl ester (0.05 g, 0.15 mmol) in THF:H₂O (1:1, 5 mL) at 0° C. was added a solution of LiOH (0.01 g, 0.46 mmol) in water (1 mL). The solution was allowed to warm to room temperature over 3 hours then acidified to pH 2 with 2N HCl at 0° C. The mixture was extracted with EtOAc (2×10 mL) and the organic extracts dried and concentrated in vacuo. The residue was purified by preparative HPLC to give [6-(5-amino-[1,3,4]thiadiazol-2-yloxy)-1-hydroxy-1,3-dihydro-benzo[c][1,2]oxaborol-3-yl]-acetic acid (0.035 g, 76%). ¹H NMR (400 MHz, DMSO): δ 7.58-7.53 (m, 2H), 7.40 (dd, J=2.4, 8 Hz, 1H), 5.45 (dd, J=4, 8.8 Hz, 1H), 2.96 (dd, J=4, 16 Hz, 1H), 2.39-2.33 (m, 1H). MS (ESI) m/z=308 [M+H]+.

### E83 6-Cyclopentyloxy-3-methanesulfonylmethyl-3H-benzo[c][1,2]oxaborol-1-ol

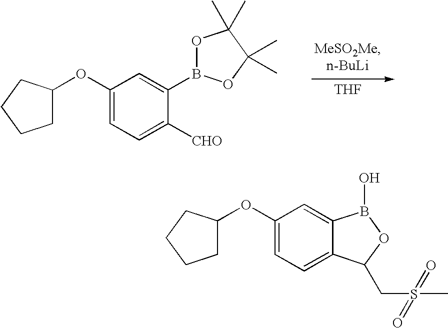

To a solution of dimethylsulfone (0.39 g, 4.17 mmol) in THF (10 mL) was added n-BuLi (2.6 mL, 4.17 mmol). White precipitate crashed out. The reaction mixture was heated to reflux for 1.5 h. The mixture was cooled to 0° C., a solution of 4-cyclopentyloxy-2-(4,4,5,5-tetramethyl-[1,3,2]dioxaborolan-2-yl)-benzaldehyde (1.2 g, 3.79 mmol) in 5 mL of THF was added to the reaction mixture and stirred at room temperature for 30 min, the reaction mixture was quenched with water and acidified to pH˜3 with 6M HCl, extracted with EtOAc and washed with water, brine, dried over Na₂SO₄, and concentrated under reduced pressure to give crude product, which was purified by biotage (50-100% EtOAc in hexane) to afford 6-cyclopentyloxy-3-methanesulfonylmethyl-3H-benzo[c][1,2]oxaborol-1-ol (0.15 g, 12% yield) as a white solid. Mp 132-134° C. ¹H NMR (400 MHz, DMSO-d₆) δ 9.38 (s, 1H), 7.48 (d, J=8.4 Hz, 1H), 7.22 (d, J=2.4 Hz, 1H), 7.03 (dd, J=8.4, 2.4 Hz, 1H), 5.40 (dd, J=10.4, 2.0 Hz, 1H), 4.90 (m, 1H), 3.76 (d, J=14.8 Hz, 1H), 3.40 (d, J=10.8 Hz, 1H), 3.10 (s, 3H), 2.00-1.82 (m, 2H), 2.80-2.68 (m, 4H), 1.62-1.55 (m, 2H). MS (ESI) m/z=309 [M−H]⁻.

### E84 4-(3-Ethoxycarbonylmethyl-1-hydroxy-1,3-dihydro-benzo[c][1,2]oxaborol-6-yloxy)-piperidine-1-carboxylic acid tert-butyl ester

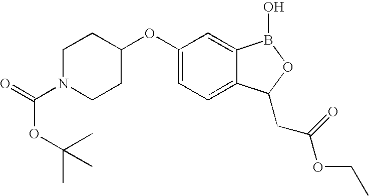

To a suspension of zinc dust (3.18 g, 48 mmol) in THF (30 mL) was added trimethylsilyl chloride (0.69 g, 6.4 mmol) at 40° C. The mixture was heated to 55° C. and stirred for 15 min. After cooling down to 37° C., ethyl bromoacetate (7.48 g, 48 mmol) was slowly added to the reaction mixture at 37-40° C. After addition, the resulting mixture was allowed to cool to room temperature over 30 minutes then cooled down to 0° C. 4-[4-formyl-3-(4,4,5,5-tetramethyl-[1,3,2]dioxaborolan-2-yl)-phenoxy]-piperidine-1-carboxylic acid tert-butyl ester (1.4 g, 3.2 mmol) in THF (10 mL) was added to the zinc solution at 0° C. The mixture was allowed to warm to room temperature over 1.5 hours before treating with saturated NH₄Cl (20 mL) and extracting with EtOAc (3×100 mL). The organic extracts were washed with brine, dried and concentrated in vacuo. The residue was purified by silica gel flash column chromatography (10-50% ethyl acetate/hexane) to get 4-(3-ethoxycarbonylmethyl-1-hydroxy-1,3-dihydro-benzo[c][1,2]oxaborol-6-yloxy)-piperidine-1-carboxylic acid tert-butyl ester (1.07 g, 81%). mp 79.2-80.5° C. ¹H NMR 400 MHz (DMSO-d₆) δ 9.18 (s, 1H), 7.34 (d, J=8.0 Hz, 1H), 7.23(s, 1H), 7.07 (d, J=8.8 Hz, 1H), 5.40 (m, 1H), 4.53-4.51 (m, 1H), 4.12-4.06 (m, 2H), 3.64 (m, 2H), 3.18 (m, 2H), 3.02-2.97 (m, 1H), 2.40-2.34 (m, 1H), 1.88 (m, 2H), 1.53 (m, 2H), 1.40 (s, 9H), 1.18 (t, J=7.6 Hz, 3H). HPLC purity: 96.30% (Maxplot), 96.09% (220 nm).

### E85 4-(3-Carboxymetyl-1-hydroxy-1,3-dihydro-benzo[c][1,2]oxaborol-6-yloxy)-piperidine-1-carboxylic acid tert-butyl ester

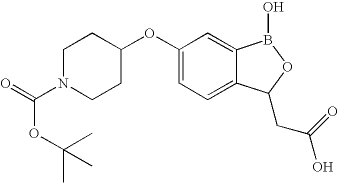

To a solution of 4-(3-ethoxycarbonylmethyl-1-hydroxy-1,3-dihydro-benzo[c][1,2]oxaborol-6-yloxy)-piperidine-1-carboxylic acid tert-butyl ester (0.07 g, 0.167 mmol) in MeOH (0.5 mL) was added a solution of LiOH (0.02 g, 0.84 mmol) in water (0.5 mL) at 0° C. The resulting mixture was stirred at 0° C. for 5 h. The reaction mixture was acidified to pH 2 using 6M hydrochloric acid and stirred at room temperature for 2 h. The precipitate was filtered and dried to give 4-(3-carboxymethyl-1-hydroxy-1,3-dihydro-benzo[c][1,2]oxaborol-6-yloxy)-piperidine-1-carboxylic acid tert-butyl ester (0.025 g, 38%) as a white solid. mp 188.5-190.2° C. ¹H NMR 400 MHz (DMSO-d₆) δ 9.15 (s, 1H), 7.35 (d, J=8.4 Hz, 1H), 7.23 (s, 1H), 7.07 (d, J=6.4 Hz, 1H), 5.39 (m, 1H), 4.50 (m, 1H), 3.65 (m, 2H), 3.18 (m, 2H), 2.88 (m, 1H), 2.32-2.25 (m, 1H), 1.98 (m, 2H), 1.53 (m, 2H), 1.40 (s, 9H). MS (ESI) m/z: 390 [M−1]⁻. HPLC purity: 97.98% (Maxplot), 97.82% (220 nm).

### E86 [1-Hydroxy-6-(piperidin-4-yloxy)-1,3-dihydro-benzo[c][1,2]oxaborol-3-yl)-acetic acid

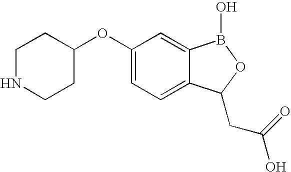

A solution of 4-(3-carboxymethyl-1-hydroxy-1,3-dihydro-benzo[c][1,2]oxaborol-6-yloxy)-piperidine-1-carboxylic acid tert-butyl ester (0.13 g, 0.33 mmol) and 4M HCl (0.99 mL, 3.98 mmol) in dioxane was stirred at room temperature for 2 hours then concentrated in vacuo. The residue was purified by preparative HPLC to give [1-hydroxy-6-(piperidin-4-yloxy)-1,3-dihydro-benzo[c][1,2]oxaborol-3-yl)-acetic acid (0.0.045 g, 46.8%) as a white solid. mp 97.8-98.2° C. 1HNMR (400 MHz, DMSO-d6) δ 9.17 (s, 1H), 7.37 (d, J=8.4 Hz, 1H), 7.25 (d, J=2.0 Hz, 1H), 7.11 (d, J=2.4 Hz, 1H), 5.38 (m, 1H), 4.60 (m, 1H), 3.21 (m, 2H), 3.05 (m, 2H), 2.88 (m, 1H), 2.32 (m, 1H), 2.08 (m, 2H), 1.90 (m, 2H). MS (ESI) m/z: 292 [M+1]+. HPLC purity: 97.36% (Maxplot), 98.40% (220 nm).

### E87 [1-Hydroxy-6-(piperidin-4-yloxy)-1,3-dihydro-benzo[c][1,2]oxaborol-3-yl)-acetic acid ethyl ester

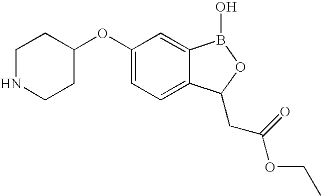

To a solution of 4-(3-ethoxycarbonylmethyl-1-hydroxy-1,3-dihydro-benzo[c][1,2]oxaborol-6-yloxy)-piperidine-1-carboxylic acid tert-butyl ester (0.37 g, 0.88 mmol) in dichloromethane (10 mL) at 0° C. was bubbled HCl (g) for 10 min. The reaction mixture was stirred at room temperature for 1 h and concentrated to give [1-hydroxy-6-(piperidin-4-yloxy)-1,3-dihydro-benzo[c][1,2]oxaborol-3-yl)-acetic acid ethyl ester 0.28 g, 89%) as a hydrochloride salt. mp 164.9-165.3° C. ¹HNMR (400 MHz, DMSO-d₆) δ 9.20 (s, 1H), 8.82 (brs, 1H), 7.35 (d, J=8.0 Hz, 1H), 7.25 (d, J=2.4 Hz, 1H), 7.10 (dd, J=8.8, 2.8 Hz, 1H), 5.38 (m, 1H), 4.60 (m, 1H), 4.10 (q, J=8.8 Hz, 2H), 3.25 (m, 2H), 3.05 (m, 2H), 3.00 (m, 1H), 2.39 (m, 1H), 2.08 (m, 2H), 1.90 (m, 2H), 1.17 (t, J=6.8 Hz, 3H). MS (ESI) m/z: 320 [M+1]⁺. HPLC purity: 93.99% (Maxplot), 91.38% (220 nm).

### E88 4-(1-Hydroxy-3-methanesulfonylmethyl-1,3-dihydro-benzo[c][1,2]oxaborol-6-yloxy)-piperidine-1-carboxylic acid tert-butyl ester

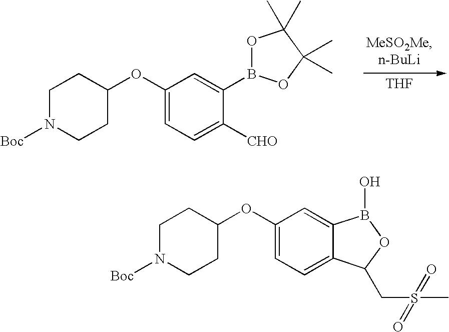

To a solution of dimethylsulfone (0.5 g, 5.2 mmol) in THF (10 mL) was added n-BuLi (2.5 M in hexane) (1.54 mL, 3.8 mmol). White precipitate crashed out. The reaction mixture was heated to reflux for 1 h. The mixture was cooled to −78° C., a solution of 4-[4-formyl-3-(4,4,5,5-tetramethyl-[1,3,2]dioxaborolan-2-yl)-phenoxy]-piperidine-1-carboxylic acid tert-butyl ester (1.5 g, 3.5 mmol) in 10 mL of THF was added to the reaction mixture and stirred at room temperature for 30 min, the reaction mixture was quenched with saturated ammonium chloride, extracted with EtOAc and washed with water, brine, dried over Na₂SO₄, and concentrated under reduced pressure to give crude product, which was purified by biotage (5-100% EtOAc in hexane) to afford the title compound (0.97 g, 71% yield) as a light yellow solid. Mp 150.9-153° C. ¹H NMR (400 MHz, DMSO-d₆) δ 9.38 (s, 1H), 7.50 (d, J=8.4 Hz, 1H), 7.27 (d, J=2.4 Hz, 1H), 7.12 (dd, J=8.8, 2.8 Hz, 1H), 5.40 (dd, J=10.4, 2.0 Hz, 1H), 4.56 (m, 1H), 3.76 (d, J=14.6 Hz, 1H), 3.68-3.63 (m, 2H), 3.40 (m, 1H), 3.27-3.18 (m, 2H), 3.16 (s, 3H), 1.98-1.88 (m, 2H), 1.56-1.48 (m, 2H), 1.40 (s, 9H). MS (ESI) m/z=424 [M−H]⁻.

### E89 3-Methanesulfonylmethyl-6-(piperidin-4-yloxy)-3H-benzo[c][1,2]oxaborol-1-ol

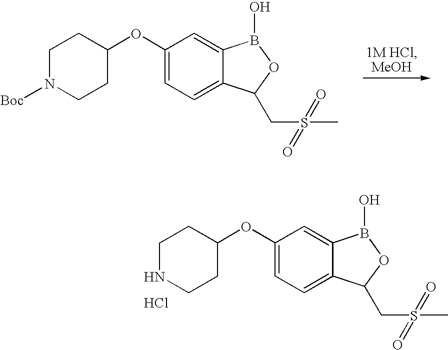

To a solution of 4-(1-hydroxy-3-methanesulfonylmethyl-1,3-dihydro-benzo[c][1,2]-oxaborol-6-yloxy)-piperidine-1-carboxylic acid tert-butyl ester (0.57 g, 1.34 mmol) in methanol (5 mL) was added 1M HCl in ether (4.4 mL, 4.37 mmol). The reaction mixture was stirred at room temperature for 3 h, and concentrated to 3-methanesulfonylmethyl-6-(piperidin-4-yloxy)-3H-benzo[c][1,2]oxaborol-1-ol hydrochloric salt (0.45 g, 93% yield) as an off white solid. Mp 234-236° C. ¹H NMR (400 MHz, DMSO-d₆) δ 9.40 (s, 1H), 8.69 (br s, 1H), 7.52 (d, J=8.8 Hz, 1H), 7.30 (d, J=2.4 Hz, 1H), 7.16 (dd, J=8.8, 2.8 Hz, 1H), 4.44 (d, J=8.4 Hz, 1H), 4.65 (m, 1H), 3.78 (d, J=14.8 Hz, 1H), 3.36 (d, J=14.4 Hz, 1H), 3.26-3.21 (m, 2H), 3.09 (s, 3H), 3.07-3.03 (m, 2H), 2.11-2.08 (m, 2H), 1.84-1.81 (m, 2H). MS (ESI) m/z=326 [M−H]⁻.

### E90 (6-Benzyl-1-hydroxy-1,3-dihydro-benzo[c][1,2]oxaborol-3-yl)-acetic acid ethyl ester

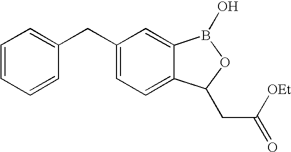

### (6-Benzyl-1-Hydroxy-1,3-dihydro-benzo[c][1,2]oxaborol-3-yl)-acetic acid ethyl ester

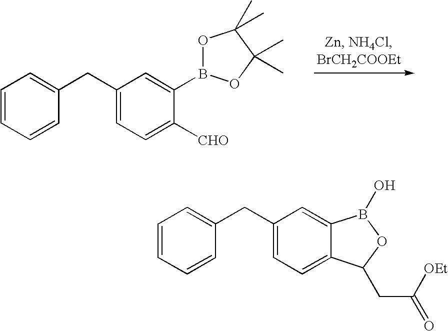

A mixture of 4-benzyl-2-(4,4,5,5-tetramethyl-[1,3,2]dioxaborolan-2-yl)-benzaldehyde (0.32 g, 1.0 mmol), ethyl bromoacetate (0.84 g, 5.0 mmol), zinc dust (1.30 g, 20 mmol) and NH₄Cl (0.54 g, 10 mmol) was thoroughly grounded in a mortar and pestle. The resulting mixture was kept at room temperature (20° C.) for 3.5 h. The mixture was treated with sat. NH₄Cl (50 mL) and extracted with ether (3×50 mL). The extract was washed with brine, dried and concentrated to dryness. The residue was purified by chromatography on silica gel to give 140 mg (45% yield) of (6-benzyl-1-hydroxy-1,3-dihydro-benzo[c][1,2]oxaborol-3-yl)-acetic acid ethyl ester as a colorless oil. ¹HNMR (400 MHz, CD₃OD) δ 7.50 (s, 1H), 7.40-71.0 (m, 7H), 5.56 (m, 1H), 4.16 (m, 2H), 3.98 (q, 2H), 2.90 (m, 1H), 2.50 (m, 1H), 1.20 (t, J=6.5 Hz, 3H).

### E91 (6-Benzyl-1-hydroxy-1,3-dihydro-benzo[c][1,2]oxaborol-3-yl)-acetic acid

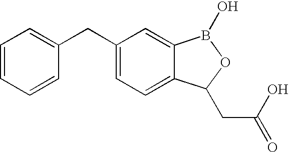

### (6-Benzyl-1-hydroxy-1,3-dihydro-benzo[c][1,2]oxaborol-3-yl)-acetic acid

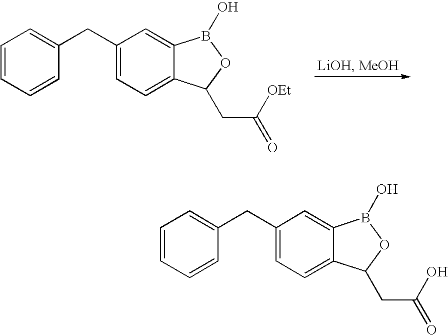

To a solution of (6-benzyl-1-hydroxy-1,3-dihydro-benzo[c][1,2]oxaborol-3-yl)-acetic acid ethyl ester (140 mg, 0.45 mmol) in methanol (5 mL) was added aqueous LiOH—H₂O (54 mg in 5 mL of water, 2.26 mmol) at 0° C. The resulting mixture was stirred at RT for 5 h. The reaction mixture was acidified to pH 2 using diluted hydrochloric acid. The mixture was extracted with EtOAc (2×20 mL). The extract was washed with brine and dried to give the crude product which was purified by chromatography on silica gel to give 0.11 (86% yield) of pure product as white powder; mp 187-189° C. ¹HNMR (DMSO-d₆, 400 MHz) δ 9.19 (s, 1H), 7.53 (s, 1H), 7.40-7.10 (m, 7H), 4.40 (m, 1H), 3.97 (s, 2H), 2.90 (m, 1H), 2.30 (m, 1H). MS (ESI) m/z=281 [M−H]⁻.

### E92 6-Benzyl-3-methanesulfonylmethyl-3H-benzo[c]oxaborol-1-ol

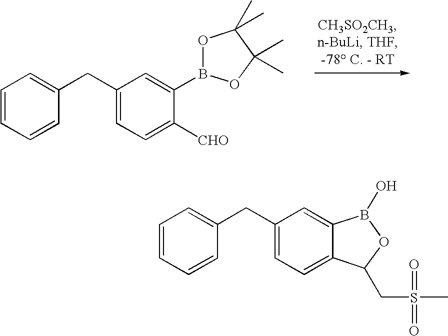

To a solution of dimethylsulfone (197 mg, 2.1 mmol) in THF (6 mL) was added n-BuLi (0.66 mL, 2.5 M in hexane) in an oven-dried three-neck flask under N₂ atmosphere. The resulting suspension was heated at 90° C. for 1 h and then cooled to −78° C. A solution of 4-benzyl-2-(4,4,5,5-tetramethyl-[1,3,2]dioxaborolan-2-yl)benzaldehyde in THF (10 mL) was added. The cooling bath was removed. The reaction mixture was gradually warmed up to RT and stirred at RT for 30 min, quenched with H₂O and acidified to pH 3 with 6 N HCl, extracted with ethyl acetate, dried and concentrated. The residue was purified by chromatography to give 6-benzyl-3-methanesulfonylmethyl-3H-benzo[c]oxaborol-1-ol (380 mg, 80% yield). Mp 152-154° C. ¹HNMR (DMSO-d₆, 400 MHz) δ 9.40 (s, 1H), 7.58 (s, 1H), 7.50 (m, 1H), 7.40 (m, 1H), 7.30-7.10 (m, 5H), 5.22 (m, 1H), 4.00 (s, 2H), 3.75 (m, 1H), 3.40 (m, 1H), 3.08 (s, 3H). MS (ESI) m/z=315 [M−H]⁻.

### E93 2-Cyano-N-(1-hydroxy-1,3-dihydro-benzo[c][1,2]oxaborol-6-yl)-benzenesulfonamide

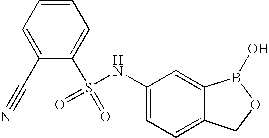

E93 was prepared using a procedure similar to that of E95. LCMS (m/z) 315 [M+H]; ¹H NMR (400 MHz, DMSO-d6) δ ppm 4.89 (s, 2H) 7.19 (dd, J=8.2, 2.1 Hz, 1H) 7.30 (d, J=8.2 Hz, 1H) 7.45 (d, J=1.8 Hz, 1H) 7.81 (dd, J=7.6, 1.2 Hz, 1H) 7.88 (td, J=7.7, 1.4 Hz, 1H) 7.99 (dd, J=8.0, 1.0 Hz, 1H) 8.06 (dd, J=7.5, 1.1 Hz, 1H) 9.22 (s, 1H) 10.74 (s, 1H).

### E93 Alternate Synthesis

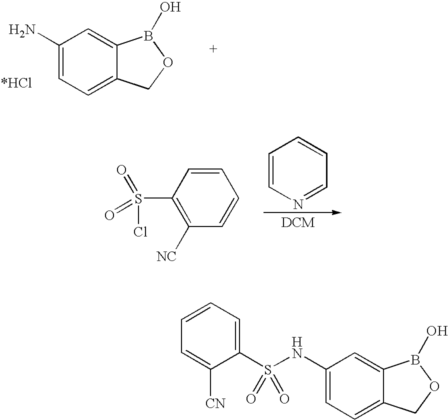

A 40 mL scintillation vial was charged with 5-amino-2-hydroxymethylphenylboronic acid hydrochloride (100 mg, 0.54 mmol, 1 eq) in dry DCM (10 mL). Pyridine (100 μl, 1.2 mmol, 2.2 eq) was then added followed by 2-cyanobenzenesulfonylchloride (135 mg, 0.67 mmol, 1.2 eq). The mixture was allowed to stir at room temperature overnight. Aqueous hydrochloric acid (1 M, 3 mL) was added and the resulting mixture was extracted twice with DCM (5 mL). The combined organic phases were dried over sodium sulfate, and the material was concentrated under reduced pressure. The residue was purified by silica gel chromatography to furnish E93 as a white solid. LCMS (m/z) 315 [M+H]; ¹H NMR (400 MHz, DMSO-d₆) δ ppm 4.89 (s, 2H) 7.19 (dd, J=8.2, 2.1 Hz, 1H) 7.30 (d, J=8.2 Hz, 1H) 7.45 (d, J=1.8 Hz, 1H) 7.81 (dd, J=7.6, 1.2 Hz, 1H) 7.88 (td, J=7.7, 1.4 Hz, 1H) 7.99 (dd, J=8.0, 1.0 Hz, 1H) 8.06 (dd, J=7.5, 1.1 Hz, 1H) 9.22 (s, 1H) 10.74 (s, 1H).

### E94 3-Cyano-N-(1-hydroxy-1,3-dihydro-benzo[c][1,2]oxaborol-6-yl)-benzenesulfonamide

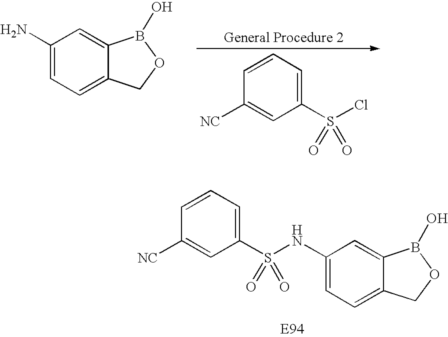

General procedure 2: 6-amino-3H-benzo[c][1,2]oxaborol-1-ol (1.72 g, 11.56 mmol), 3-cyano-benzenesulfonyl chloride (2.33 g, 11.56 mmol), pyridine (2.8 mL, 34.68 mmol), and MeCN (20 mL). Purification: flash chromatography (95:5 CH₂Cl₂/MeOH): yield 1.5 g (41%). ¹H NMR (400 MHz, DMSO-d₆) δ (ppm): 10.45 (bs, 1H), 9.25 (s, 1H), 8.05-8.21 (m, 2H), 7.91-8.04 (m, 1H), 7.77 (t, J=7.8 Hz, 1H), 7.47 (d, J=1.6 Hz, 1H), 7.30 (d, J=8.2 Hz, 1H), 7.17 (dd, J=8.2, 2.0 Hz, 1H), 4.90 (s, 2H); MS (ESI) m/z=313 (M−1, negative); HPLC: 95.49% (220 nm), 95.15% (254 nm).

### E95 4-Cyano-N-(1-hydroxy-1,3-dihydro-benzo[c][1,2]oxaborol-6-yl)-benzenesulfonamide

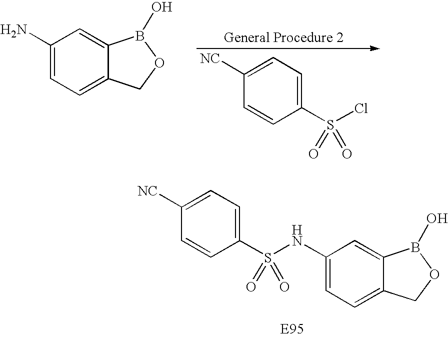

General procedure 2: 6-amino-3H-benzo[c][1,2]oxaborol-1-ol (775 mg, 5.21 mmol), 4-cyano-benzenesulfonyl chloride (1.05 g, 5.21 mmol), pyridine (1.29 mL, 15.6 mmol), and MeCN (20 mL). Purification: flash chromatography (95:5 CH₂Cl₂/MeOH): yield 1.2 g (74%). ¹H NMR (400 MHz, DMSO-d₆) δ (ppm): 10.49 (bs, 1H), 9.22 (s, 1H), 8.02 (d, J=8.6 Hz, 2H), 7.85 (d, J=8.6 Hz, 2H), 7.45 (s, 1H), 7.27 (d, J=8.2 Hz, 1H), 7.14 (dd, J=7.8, 2.0 Hz, 1H), 5.74 (s, 7H), 4.87 (s, 2H); MS (ESI) m/z=313 (M−1, negative); HPLC: 96.56% (220 nm), 90.98% (254 nm).

### E96 2-Aminomethyl-N-(1-hydroxy-1,3-dihydro-benzo[c][1,2]oxaborol-6-yl)-benzenesulfonamide hydrochloride

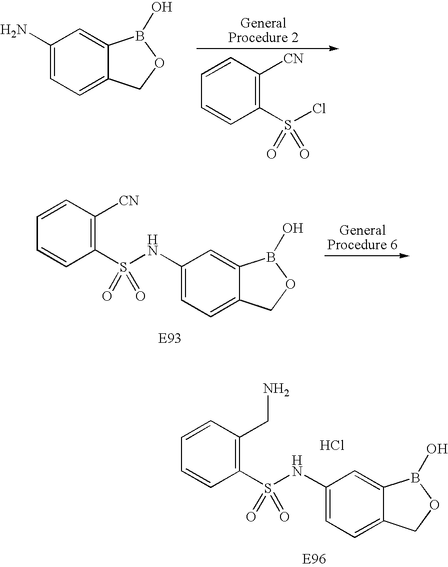

General procedure 2: 6-amino-3H-benzo[c][1,2]oxaborol-1-ol (1.51 mg, 10.1 mmol), 2-cyano-benzenesulfonyl chloride (2.05 g, 10.1 mmol), pyridine (2.5 mL, 30.3 mmol), and MeCN (20 mL). The resulting 2-cyano-N-(1-hydroxy-1,3-dihydro-benzo[c][1,2]oxaborol-6-yl)-benzene sulfonamide (E93) was used directly without further purification. ¹H NMR (400 MHz, DMSO-d₆) δ (ppm): 10.77 (s, 1H), 9.25 (s, 1H), 8.07 (d, J=7.9 Hz, 1H), 7.98 (d, J=7.9 Hz, 1H), 7.88 (dd, J=7.9 Hz, 7.3 Hz, 1H), 7.80 (dd, J=8.2 Hz, 7.9 Hz, 1H), 7.45 (s, 1H), 7.30 (d, J=8.2 Hz, 1H), 7.19 (d, J=7.3 Hz, 1H), 4.89 (s, 2H); MS (ESI) m/z=313 (M−H)⁻.

General procedure 6: 2-cyano-N-(1-hydroxy-1,3-dihydro-benzo[c][1,2]oxaborol-6-yl)-benzene sulfonamide (500 mg, 1.59 mmol), Raney Ni (1 g), and 7 M NH₃ in MeOH (20 mL): H₂ (50 psi) at rt for 5 h. Purification: precipitation: yield 398 mg (80%) of E96. ¹H NMR (400 MHz, DMSO-d₆) δ (ppm): 10.69 (s, 1H), 8.56 (bs, 3H), 7.77 (d, J=7.8 Hz, 1H), 7.67 (d, J=3.5 Hz, 2H), 7.49 (s, 2H), 7.26 (d, J=8.2 Hz, 1H), 7.21-7.11 (m, 1H), 4.86 (s, 2H), 4.38 (d, J=5.1 Hz, 2H); MS (ESI) m/z=319 (M+1, positive); HPLC: 96.12% (220 nm), 95.11% (MaxPlot).

### E97 3-Aminomethyl-N-(1-hydroxy-1,3-dihydro-benzo[c][1,2]oxaborol-6-yl)-benzenesulfonamide

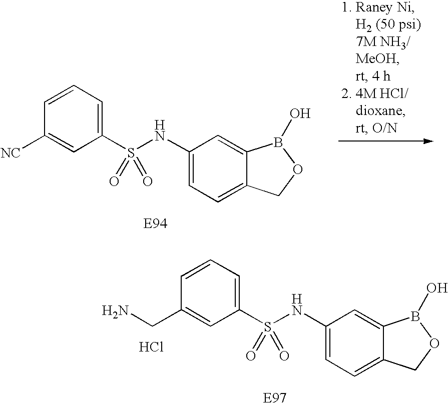

A mixture of 3-cyano-N-(1-hydroxy-1,3-dihydro-benzo[c][1,2]oxaborol-6-yl)-benzene sulfonamide (700 mg, 2.23 mmol), Raney Ni (1 g), and 7 M NH₃ in MeOH (20 mL) was hydrogenated at 50 psi at rt for 5 h. After filtration, the filtrate was concentrated in vacuo. The residue was dissolved in MeOH (3 mL) and a solution of 4 M HCl in dioxane (10 mL) was added. After stirring for 30 min, dioxane (10 mL) was added to initiate crystallization. After stirring O/N, the precipitate was filtered off The filtrate was mixed with H₂O (15 mL) and then lyophilized to give E97: yield 550 mg (70%). ¹H NMR (400 MHz, DMSO-d₆) δ (ppm): 10.45 (s, 1H), 9.26 (s, 1H), 8.47 (bs, 3H), 7.97 (s, 1H), 7.76 (dd, J=12.09, 7.8 Hz, 2H), 7.60 (t, J=7.8 Hz, 1H), 7.53 (d, J=1.6 Hz, 1H), 7.40 (s, 1H), 7.27 (d, J=7.8 Hz, 2H), 7.19-7.23 (m, 1H), 7.15 (s, 1H), 4.88 (s, 2H), 4.08 (d, J=5.1 Hz, 2H); MS (ESI) m/z=319 (M+1, positive); HPLC: 95.61% (220 nm), 90.95% (254 nm).

### E98 4-Aminomethyl-N-(1-hydroxy-1,3-dihydro-benzo[c][1,2]oxaborol-6-yl)-benzenesulfonamide

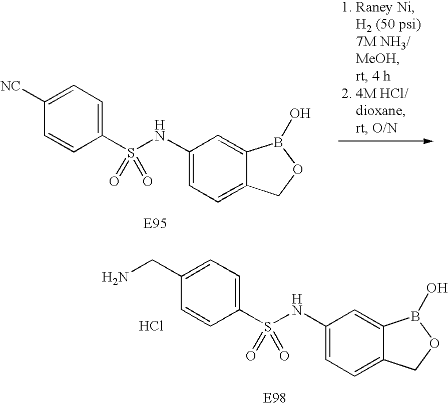

A mixture of 4-cyano-N-(1-hydroxy-1,3-dihydro-benzo[c][1,2]oxaborol-6-yl)-benzene sulfonamide (600 mg, 1.91 mmol), Raney Ni (1 g), and 7 M NH₃ in MeOH (20 mL) was hydrogenated at 50 psi at rt for 5 h. After filtration, the filtrate was concentrated in vacuo. The residue was dissolved in MeOH (3 mL) and a solution of 4 M HCl in dioxane (10 mL) was added. After stirring for 30 min, dioxane (10 mL) was added to initiate crystallization. After stirring O/N, the precipitate was filtered off The filtrate was mixed with H₂O (15 mL) and then lyophilized to give E98: yield 520 mg (76%). ¹H NMR (400 MHz, DMSO-d₆) δ (ppm): 10.40 (s, 1H), 9.22 (s, 1H), 8.42 (bs, 3H), 7.80 (d, 2H), 7.63 (d, 2H), 7.53 (s, 1H), 7.26-7.19 (m, 2H), 4.86 (s, 2H), 4.03 (s, 2H); MS (ESI) m/z=319 (M+1, positive); HPLC: 93.58% (220 nm), 90.3% (254 nm)

### E99 N-(1-hydroxy-1,3-dihydrobenzo[c][1,2]oxaborol-6-yl)-3-(hydroxymethyl)benzenesulfonamide

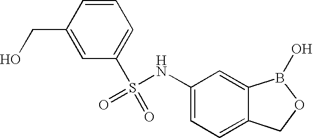

General Procedure 2: Starting Materials 6-amino-3H-benzo[c][1,2]oxaborol-1-ol and 3-(hydroxymethyl)benzene-1-sulfonyl chloride. ¹H NMR (300 MHz, DMSO-d₆) δ (ppm): 10.41 (s, 1H), 9.07 (s, 1H), 7.77 (s, 1H), 7.61-7.59 (d, 1H), 7.53-7.50 (d, 1H), 7.44-7.42 (m, 2H), 7.32 (m, 1H), 7.21(m, 1H), 5.35 (bs, 1H), 4.96 (s, 2H), 4.59 (s, 2H); MS (ESI): m/z=320.0 (M+1, positive); HPLC purity: 100% (254 nm), 94.1% (220 nm).

### E100 N-(1-hydroxy-1,3-dihydrobenzo[c][1,2]oxaborol-6-yl)-4-(hydroxymethyl)benzenesulfonamide

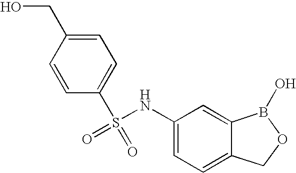

General Procedure 2: Starting Materials 6-amino-3H-benzo[c][1,2]oxaborol-1-ol and 4-(hydroxymethyl)benzene-1-sulfonyl chloride. ¹H NMR (300 MHz, DMSO-d₆) δ (ppm): 10.41 (s, 1H), 9.07 (s, 1H), 7.68-7.66 (d, 2H), 7.37-7.35 (m, 2H), 7.13-7.05 (m, 2H), 6.84-6.81 (m, 1H), 5.32 (bs, 1H), 4.82 (s, 2H), 4.49 (s, 2H); MS (ESI): m/z=320.0 (M+1, positive); HPLC purity: 95.7% (254 nm), 94.7% (220 nm).

### E101 4-Acetyl-N-(1-hydroxy-1,3-dihydro-benzo[c][1,2]oxaborol-6-yl)-benzenesulfonamide

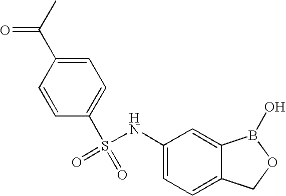

E101 was prepared using a procedure similar to that of E95. LCMS (m/z) 332 [M+H]; ¹H NMR (400 MHz, DMSO- d6) δ ppm 2.58 (s, 3H) 4.88 (s, 2H) 7.18 (dd, J=8.2, 2.1 Hz, 1H) 7.27 (d, J=8.4 Hz, 1H) 7.49 (d, J=2.1 Hz, 1H) 7.85 (d, J=8.6 Hz, 2H) 8.07 (d, J=8.6 Hz, 2H) 9.22 (s, 1H) 10.42 (s, 1H).

### E102 2-(1-Hydroxy-1,3-dihydro-benzo[c][1,2]oxaborol-6-ylsulfamoyl)-benzoic acid methyl ester

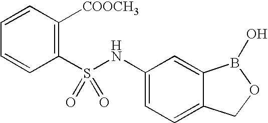

General Procedure 2: 6-amino-3H-benzo[c][1,2]oxaborol-1-ol (1.00 g, 6.71 mmol), acetonitrile (20 mL), 2-chlorosulfonyl-benzoic acid methyl ester (1.89 g, 8.06 mmol), N-methyl morpholine (2.71 g, 26.85). Column purification followed by HPLC purification in MeOH and 0.1% formic acid/water afforded 124 mg (0.35 mmol, 5%) of the title compound as white solid. ¹H NMR (400 MHz, DMSO-d₆) δ ppm 10.09 (s, 1H), 9.22 (s, 1H), 7.88-7.79 (m, 1H), 7.73-7.58 (m, 3H), 7.49 (s, 1H), 7.28 (d, J=8.2 Hz, 1H), 7.20 (dd, J=8.2, 1.6 Hz, 1H), 4.89 (s, 2H), 3.84 (s, 3H); MS (ESI) m/z=346 (M−1, negative); HPLC purity: 97.99% (MaxPlot 200-400 nm), 98.11% (220 nm); Anal. Calcd for C₁₅H₁₄BNO₆S: C 51.90%; H 4.06%; N 4.03%. Found: C 51.77%; H 4.16%; N 4.48%.

### E103 2-(1-hydroxy-1,3-dihydro-benzo[c][1,2]oxaborol-6-ylsulfamoyl)-benzoic acid

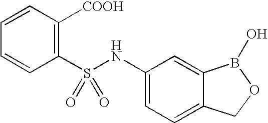

To a stirred solution of 2-(1-hydroxy-1,3-dihydro-benzo[c][1,2]oxaborol-6-ylsulfamoyl)-benzoic acid methyl ester (800 mg, 2.30 mmol) in MeOH (40 mL) was added LiOH (670 mg, 27.91 mmol in 15 mL water). After overnight, the reaction mixture was cooled in an ice bath and acidified using 2N HCl. The aqueous layer was extracted with DCM (3×50 mL), and the combined organic layer was dried over MgSO₄ and filtered. Flash column chromatography in MeOH/DCM (1 to 5%) and then preparative HPLC (50×100 Gem 10μ) in 30 to 90% acetonitrile in water afforded 110 mg (0.33 mmol, 14%) of the title compound as a white solid. ¹H NMR (400 MHz, DMSO-d₆) δ ppm 9.19 (s, 1H), 7.71 (d, J=7.4 Hz, 1H), 7.69-7.59 (m, 2H), 7.57-7.50 (m, 1H), 7.46 (s, 1H), 7.25 (d, J=8.2 Hz, 1H), 7.23-7.17 (m, 1H), 4.86 (s, 2H); MS (ESI) m/z=332 (M−1, negative); HPLC purity: 96.00% (MaxPlot 200-400 nm), 95.67% (220 nm); Anal. Calcd for C₁₄H₁₂BNO₆S.0.33H₂O: C 49.58%; H 3.76%; N 4.13%. Found: C 49.60%; H 3.78%; N 4.31%.

### E104 4-(1-Hydroxy-1,3-dihydro-benzo[c][1,2]oxaborol-6-ylsulfamoyl)-benzoic acid methyl ester

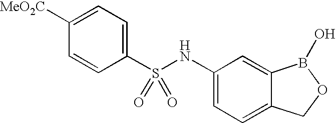

General Procedure 2: 6-amino-3H-benzo[c][1,2]oxaborol-1-ol (536 mg, 3.6 mmol), 4-chlorosulfonylbenzoic acid methyl ester (1.0 g, 4.3 mmol), NMM (1.5 mL, 14.2 mmol), and MeCN (10 mL) at rt O/N. The mixture was concentrated in vacuo. H₂O (7.5 mL) was added and the mixture was then acidified with 1 M HCl (5 drops). EtOAc (15 mL) was added and the mixture was stirred until a clear biphasic solution was observed. The aqueous layer was loaded onto an Isolute HM-N column and left to stand for 10 min. The organic layer was then eluted through the column. The column was further washed with EtOAc (20 mL). The organic fractions were concentrated in vacuo and the residue was dissolved in MeOH and loaded onto a pre-column (silica, 12 g). Purification by flash chromatography (20-100% EtOAc/hexane) gave a yellow solid; yield: 280 mg (22%). Recrystallization from MeCN/H₂O (2×) was followed by prep HPLC (0.1% TFA (aq)/MeCN. The major fraction was isolated, concentrated in vacuo at 40° C. and then lyophilized to give the title compound as a white solid (61 mg). ¹H NMR (400 MHz, DMSO-d₆) δ (ppm): 10.42 (bs, 1H), 9.24 (s, 1H), 8.10-8.07 (m, 2H), 7.86-7.84 (m, 2H), 7.47-7.46 (m, 1H), 7.29-7.27 (m, 1H), 7.17-7.15 (m, 1H), 4.88 (s, 2H), 3.86 (s, 3H); MS (ESI) m/z=346 (M−1, negative); HPLC purity: 98.96% (MaxPlot 200-400 nm), 98.78% (220 nm).

### E105 4-(N-(1-hydroxy-1,3-dihydrobenzo[c][1,2]oxaborol-6-yl)sulfamoyl)benzoic acid

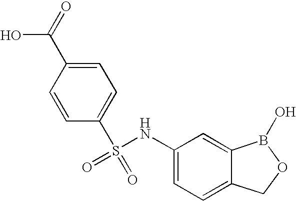

General Procedure 2: Starting Materials 6-amino-3H-benzo[c][1,2]oxaborol-1-ol and 4-(chlorosulfonyl)benzoic acid. ¹H NMR (300 MHz, DMSO-d₆) δ (ppm): 13.46 (bs, 1H), 10.41 (s, 1H), 9.23 (s, 1H), 8.07-8.04 (d, 2H), 7.85-7.82 (d, 2H), 7.48 (s, 1H), 7.29-7.26 (d, 1H), 7.19-7.16 (dd, 1H), 4.88 (s, 2H); MS (ESI): m/z=334.0 (M+1, positive); HPLC purity: 100% (254 nm), 100% (220 nm).

### E106 N-(1-Hydroxy-1,3-dihydro-benzo[c][1,2]oxaborol-6-yl)-2-methoxy-benzenesulfonamide

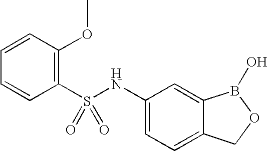

2-Methoxybenzenesulfonyl chloride (0.67 g, 3.24 mmol) was added to a mixture of 6-amino-3H-benzo[c][1,2]oxaborol-1-ol (0.5 g, 2.7 mmol) and N-methylmorpholine (1.64 g, 16.2 mmol) in anhydrous acetonitrile (10 mL) at 0° C. under nitrogen. The reaction mixture was stirred at room temperature for 18 h, diluted with ethyl acetate (100 mL), washed with water (40 mL), brine, dried over Na₂SO₄ and concentrated to give crude product as a yellow solid. Purification by silica column chromatography (eluant 3% MeOH in DCM) to afford E106 as an ivory solid (0.52 g, 60%). m.p. 177-179° C. ¹H NMR (400 MHz, DMSO) δ (ppm) 9.94 (s, 1H), 9.19 (s, 1H); 7.71 (d, J=7.6 Hz, 1H); 7.52 (t, J=7.6 Hz, 1H), 7.47 (s, 1H); 7.23-7.14 (m, 3H), 7.00 (t, J=7.8 Hz, 1H), 4.84 (s, 2H), 3.89 (s, 3H). MS(ESI): m/z=318 (M−1, negative). HPLC purity: 98.23% (Max. Plot 200-400 nm), 98.64%(220 nm).

### E107 2-Hydroxy-N-(1-hydroxy-1,3-dihydro-benzo[c][1,2]oxaborol-6-yl)-benzenesulfonamide

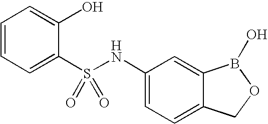

To a solution of E106 (107 mg, 0.335 mmol) in anhydrous DCM (5 mL) was added slowly BBr₃ (1M in DCM, 0.74 mL, 0.737 mmol) at −5° C. under nitrogen. The reaction mixture was stirred at 0° C. for 10 min. and at room temperature for 2 h. The reaction was poured into ice-brine (7 mL) and extracted with DCM (30 mL). The organic layer was washed with brine (2×10 mL) to pH 7, dried over Na₂SO₄ and concentrated in vacuo. Crystallization of from DCM/hexanes provided 79 mg (78%) of the title compound as a white solid. m.p. 166-168° C. ¹H NMR (400 MHz, DMSO) δ (ppm) 10.86 (s, 1H); 9.90 (s, 1H), 9.19 (s, 1H), 7.65 (d, J=8 Hz, 1H), 7.49 (s, 1H), 7.36 (t, J=8 Hz, 1H), 7.21 (s, 2H); 6.92 (d, J=8 Hz, 1H), 6.84 (t, J=8 Hz, 1H), 4.84 (s, 2H); MS(ESI): m/z=304 (M−1, negative). HPLC purity: 97.77% (Max. Plot 200-400 nm); 98.25%(220 nm).

### E108 N-(1-Hydroxy-1,3-dihydro-benzo[c][1,2]oxaborol-6-yl)-3-methoxy-benzenesulfonamide

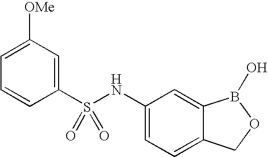

General Procedure 2: 6-amino-3H-benzo[c][1,2]oxaborol-1-ol (0.60 g, 4.0 mmol), 3-methoxybenzenesulfonyl chloride (1.0 g, 4.8 mmol), NMM (1.7 mL, 16.0 mmol) and MeCN (20 mL) at rt O/N. The mixture was concentrated in vacuo and H₂O (5 mL) and EtOAc (15 mL) were added. The mixture was stirred until a clear biphasic solution was observed. The aqueous layer was then loaded onto an Isolute HM-N column and left to stand for 10 min. The organic layer was then eluted through the column. The column was then further washed with EtOAc (20 mL). The organic fractions were concentrated in vacuo and the residue was dissolved in MeOH and loaded onto a pre-column (silica, 4 g). Purification by flash chromatography (silica, 12 g; 20-100% EtOAc/hexane) gave a colorless oil; yield: 398 mg (31%). Recrystallization from MeCN/H₂O gave the title compound as a white solid (267 mg). ¹H NMR (400 MHz, DMSO-d₆) δ (ppm): 10.24 (bs, 1H), 9.24 (s, 1H), 7.51-7.50 (m, 1H), 7.46-7.42 (m, 2H), 7.29-7.25 (m, 2H), 7.19-7.14 (m, 2H), 4.88 (s, 2H), 3.76 (s, 3H); MS (ESI) m/z=318 (M−1, negative); HPLC purity: 89.76% (MaxPlot 200-400 nm), 89.70% (220 nm).

### E108 Alternate Synthesis

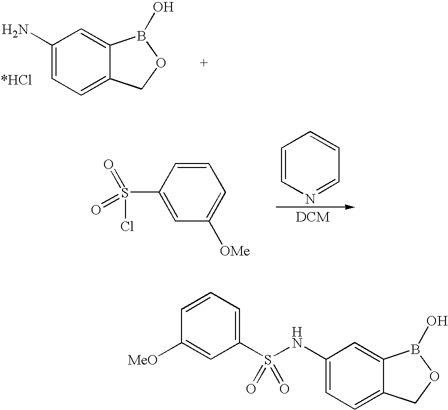

A 40 mL scintillation vial was charged with 5-amino-2-hydroxymethylphenylboronic acid hydrochloride (100 mg, 0.54 mmol, 1 eq) in dry DCM (10 mL). Pyridine (100 μl, 1.2 mmol, 2.2 eq) was then added followed by 3-methoxybenzenesulfonylchloride (95 μl, 0.67 mmol, 1.2 eq). The mixture was allowed to stir at room temperature overnight. Aqueous hydrochloric acid (1 M, 3 mL) was added and the resulting mixture was extracted twice with DCM (5 mL). The combined organic phases were dried over sodium sulfate, and the material was concentrated under reduced pressure. The residue was purified by silica gel chromatography to furnish E108 as a clear oil. LCMS (m/z) 320 [M+H]; ¹H NMR (400 MHz, DMSO-d₆) δ ppm 3.76 (s, 3H) 4.88 (s, 2H) 7.12-7.21 (m, 2H) 7.23-7.31 (m, 3H) 7.44 (t, J=8.0 Hz, 1H) 7.50 (d, J=2.0 Hz, 1H) 9.22 (s, 1H) 10.23 (s, 1H).

### E109 3-Hydroxy-N-(1-hydroxy-1,3-dihydro-benzo[c][1,2]oxaborol-6-yl)-benzenesulfonamide

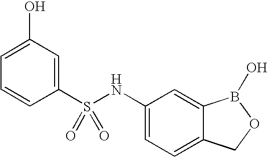

N-(1-Hydroxy-1,3-dihydro-benzo[c][1,2]oxaborol-6-yl)-3-methoxy-benzenesulfonamide (70 mg, 0.22 mmol) was dissolved in 1 M BBr₃ in CH₂Cl₂ (2.2 mL, 2.2 mmol) and the resulting solution was stirred at rt for 4 h. H₂O was then added and the mixture concentrated in vacuo at 50° C. The residue was purified by prep HPLC [MeCN/0.1% HCO₂H (aq)] and lyophilization of the major peak from 1 M HCl gave the title compound as a white solid: yield; 19 mg (28%). ¹H NMR (400 MHz, DMSO-d₆) δ (ppm): 10.56 (bs, 1H), 10.05 (s, 1H), 9.21 (s, 1H), 7.48-7.47 (m, 1H), 7.33-7.25 (m, 2H), 7.18-7.14 (m, 2H), 7.11-7.10 (m, 1H), 6.95-6.93 (m, 1H), 4.88 (s, 2H); MS (ESI) m/z=304 (M−1, negative); HPLC purity: 99.82% (MaxPlot 200-400 nm), 99.61% (220 nm).

### E110 N-(1-Hydroxy-1,3-dihydro-benzo[c][1,2]oxaborol-6-yl)-4-methoxybenzenesulfonamide

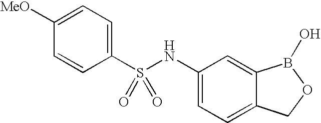

General Procedure 2: 6-amino-3H-benzo[c][1,2]oxaborol-1-ol (0.764 g, 5.13 mmol), MeCN (20 mL), NMM (2.26 mL, 20.5 mmol), and 4-methoxy-benzenesulfonyl chloride (1.16 g, 5.64 mmol). Purification: flash chromatography (95:5 EtOAc/MeOH) then recrystallization from H₂O. E110 is isolated as a white solid: yield 0.753 g (46%). mp 157-158° C.; ¹H NMR (400 MHz, DMSO-d₆) δ (ppm): 10.11 (s, 1H), 9.23 (s, 1H), 7.66 (d, J=9.0 Hz, 2H), 7.48 (d, J=2.0 Hz, 1H), 7.25 (d, J=8.2 Hz, 1H), 7.16 (dd, J=8.2, 2.0 Hz, 1H), 7.04 (d, J=9.0 Hz, 2H), 4.87 (s, 2H), 3.78 (s, 3H); HPLC purity: 95.72% (MaxPlot 200-400 nm), 96.96% (220 nm), 96.99% (254 nm); Anal. Calcd for C₁₄H₁₄BNO₅S: C 52.69%; H 4.42%; N 4.39%. Found: C 52.42%; H 4.30%; N 4.65%.

### E111 N-(1-hydroxy-1,3-dihydrobenzo[c][1,2]oxaborol-6-yl)-4-methoxybenzenesulfonamide

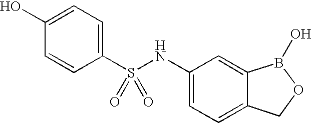

To a solution of N-(1-hydroxy-1,3-dihydrobenzo[c][1,2]oxaborol-6-yl)-4-methoxybenzenesulfonamide (E110) (0.3 g, 0.94 mmol) in DCM (10 mL), was added boron tribromide (1M solution in DCM) (2.82 ml , 2.82 mmol) and stirred at 0° C. overnight. Purification: ice was added and worked up with EtOAc; preparative HPLC was applied for the purification to give E111 as a white powder. Yield 0.973 g (34%). ¹H NMR (400 MHz, DMSO-d₆) δ (ppm): 10.38 (bs, 1H), 9.99 (s, 1H),), 9.28 (s, 1H), 7.56-7.53 (m, 2H), 7.47-7.46 (d, J=1.7 Hz, 1H), 7.24 (d, J=8.2 Hz, 1H), 7.16-7.14 (dd, J=8.1, 2.0 Hz, 1H), 6.83-6.80 (m, 2H), 4.87 (s, 2H); MS (ESI): m/z=304.1 (M−H, negative).

### E112 N-(1-hydroxy-1,3-dihydrobenzo[c][1,2]oxaborol-6-yl)-4-(trifluoromethoxy)benzenesulfonamide

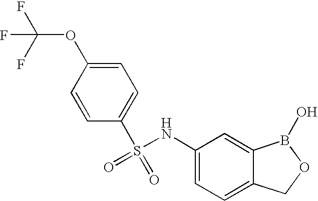

General Procedure 2: Starting Materials 6-amino-3H-benzo[c][1,2] oxaborol-1-ol and 4-(trifluoromethoxy)benzene-1-sulfonyl chloride. ¹H NMR (300 MHz, DMSO-d₆) δ (ppm): 10.37 (s, 1H), 9.22 (s, 1H), 7.86-7.83 (d, 2H), 7.56-7.49 (m, 3H), 7.30-7.28 (d, 1H), 7.19-7.16 (m, 1H), 4.89 (s, 2H); MS (ESI); m/z=374.0 (M+1, positive); HPLC purity: 100% (254 nm), 100% (220 nm).

### E113 3-Difluoromethoxy-N-(1-hydroxy-1,3-dihydro-benzo[c][1,2]oxaborol-6-yl)-benzenesulfonamide

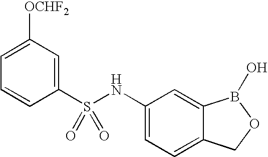

General Procedure 2: 6-Amino-3H-benzo[c][1,2]oxaborol-1-ol (150 mg, 1.0 mmol), 3-(difluoromethoxy)benzenesulfonyl chloride (300 mg, 1.2 mmol), NMM (0.43 mL, 4.0 mmol) and MeCN (5 mL) at rt O/N. The mixture was concentrated in vacuo and H₂O (5 mL) and EtOAc (15 mL) were added and the mixture was stirred until a clear biphasic solution was observed. The aqueous layer was loaded onto an Isolute HM-N column and left to stand for 10 min. The organic layer was then eluted through the column. The column was then further washed with EtOAc (20 mL). The organic fractions were concentrated in vacuo and the residue was dissolved in MeOH and loaded onto a pre-column (silica, 4 g). Purification by flash chromatography (silica, 12 g; 20-100% EtOAc/hexane) gave a yellow oil. Recrystallization from MeCN/H₂O gave the title compound as a white solid: yield; 25 mg (7%). ¹H NMR (400 MHz, DMSO-d₆) δ (ppm): 10.36 (bs, 1H), 9.22 (s, 1H), 7.60-7.53 (m, 2H), 7.46-7.44 (m, 2H), 7.41-7.39 (m, 2H), 7.26-7.24 (m, 1H), 7.16-7.14 (m, 1H), 4.86 (s, 2H); ¹⁹F NMR (376 MHz, DMSO-d₆) δ (ppm): −83.16 (s); MS (ESI) m/z=354 (M−1, negative); HPLC purity: 96.32% (MaxPlot 200-400 nm), 96.08% (220 nm).

### E114 N-(1-Hydroxy-1,3-dihydro-benzo[c][1,2]oxaborol-6-yl)-2-nitro-benzenesulfonamide

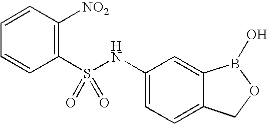

General Procedure 1: 6-amino-3H-benzo[c][1,2]oxaborol-1-ol (1.0 g, 6.7 mmol), 2-nitrobenzenesulfonyl chloride (1.8 g, 8.0 mmol), NMM (2.94 mL, 26.8 mmol) and MeCN (20 mL) at rt O/N. The mixture was concentrated in vacuo and H₂O (5 mL) and EtOAc (15 mL) were added and the mixture was stirred until a clear biphasic solution was observed. The aqueous layer was loaded onto an Isolute HM-N column and left to stand for 10 min. The organic layer was then eluted through the column. The column was then further washed with EtOAc (20 mL). The organic fractions were concentrated in vacuo and the residue was dissolved in MeOH and loaded onto a pre-column (silica, 12 g). Purification by flash chromatography (20-100% EtOAc/hexane, then 0-20% MeOH/EtOAc) gave a yellow oil; yield: 1.23 g (31%). Recrystallization of a portion of this material from MeCN/H₂O gave the title compound as a white solid (25 mg). ¹H NMR (400 MHz, DMSO-d₆) δ (ppm): 10.68 (bs, 1H), 9.27 (s, 1H), 7.98-7.92 (m, 2H), 7.85-7.77 (m, 2H), 7.51-7.50 (m, 1H), 7.33-7.31 (m, 1H), 7.22-7.21 (m, 1H), 4.90 (s, 2H); (ESI) m/z=333 (M−1, negative); HPLC purity: 95.65% (MaxPlot 200-400 nm), 95.43% (220 nm).

### E115 N-(1-Hydroxy-1,3-dihydro-benzo[c][1,2]oxaborol-6-yl)-3-nitro-benzenesulfonamide

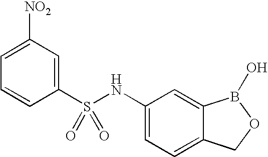

General Procedure 2: 6-amino-3H-benzo[c][1,2]oxaborol-1-ol (1.0 g, 6.7 mmol), 3-nitrobenzenesulfonyl chloride (1.8 g, 8.0 mmol), NMM (2.9 mL, 26.8 mmol), and MeCN (20 mL) at rt O/N. The mixture was concentrated in vacuo. H₂O (10 mL) was added and the resulting mixture acidified with 1 M HCl (10 drops). EtOAc (20 mL) was added and the mixture was stirred until a clear biphasic solution was observed. The aqueous layer was loaded onto an Isolute HM-N column and left to stand for 10 min. The organic layer was then eluted through the column. The column was then further washed with EtOAc (40 mL). The organic fractions were concentrated in vacuo and the residue was dissolved in MeOH and loaded onto a pre-column (silica, 12 g). Purification by flash chromatography (20-100% EtOAc/hexane) gave a yellow solid. Recrystallization from MeCN/H₂O gave a white solid. A portion of the precipitate was further purified by prep HPLC (0.1% TFA (aq)/MeCN). The major fraction was isolated, concentrated in vacuo at 40° C., and then lyophilized to give the title compound as a white solid (84 mg). ¹H NMR (400 MHz, DMSO-d₆) δ (ppm): 10.55 (bs, 1H), 9.25 (s, 1H), 8.51-8.50 (m, 1H), 8.46-8.43 (m, 1H), 8.09-8.07 (m, 1H), 7.86-7.82 (m, 1H), 7.49-7.48 (m, 1H), 7.30-7.29 (m, 1H), 7.20-7.17 (m, 1H), 4.89 (s, 2H); MS (ESI) m/z=333 (M−1, negative); HPLC purity: 99.53% (MaxPlot 200-400 nm), 99.35% (220 nm).

### E116 N-(1-Hydrox-1,3-dihydro-benzo[c][1,2]oxaborol-6-yl)-4-nitro-benzenesulfonamide

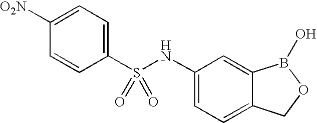

General Procedure 2: 6-amino-3H-benzo[c][1,2]oxaborol-1-ol (1 g, 6.71 mmol), 4-nitro-benzenesulfonyl chloride (1.63 g, 7.38 mmol) and NMM (2.71 g, 26.84 mmol) in acetonitrile (150 mL). The product was purified by column using 20% EtOAc in hexanes to afford the title compound (0.7 g, 31%) as a white solid. mp 166-167° C.; ¹H NMR (400 MHz, DMSO-d₆) δ ppm 10.57 (s, 1H), 9.23 (s, 1H), 8.37 (d, J=9.0 Hz, 2H), 7.96 (d, J=9.0 Hz, 2H), 7.49 (d, J=2.0 Hz, 1H), 7.30 (d, J=8.2 Hz, 1H), 7.18 (dd, J=8.2, 2.3 Hz, 1H), 4.89 (s, 2H); ESI-MS m/z 333 (M−H, negative); HPLC purity: 94.53% (MaxPlot 200-400 nm), 94.41% (220 nm).

### E116 Alternate Synthesis

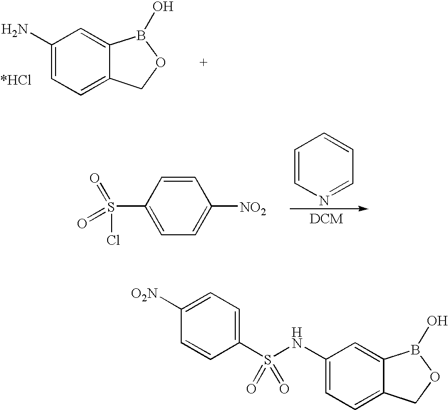

A 40 mL scintillation vial was charged with 5-amino-2-hydroxymethylphenylboronic acid hydrochloride (80 mg, 0.43 mmol, 1 eq) in dry DCM (8 mL). Pyridine (80 μl, 0.95 mmol, 2.2 eq) was then added followed by 4-nitrobenzenesulfonylchloride (115 mg, 0.52 mmol, 1.2 eq). The mixture was allowed to stir at room temperature overnight. Aqueous hydrochloric acid (1 M, 3 mL) was added and the resulting mixture was extracted twice with DCM (5 mL). The combined organic phases were dried over sodium sulfate, and the material was concentrated under reduced pressure. The residue was purified by silica gel chromatography and the appropriate fractions were combined and evaporated to afford a off white solid. Trituration with dichloromethane (2 mL) furnished E116 as a white solid. LCMS (m/z) 335 [M+H]; ¹H NMR (400 MHz, DMSO- d₆) δ ppm 4.89 (s, 2H) 7.18 (dd, J=8.2, 2.1 Hz, 1H) 7.29 (d, J=8.2 Hz, 1H) 7.49 (d, J=2.1 Hz, 1H) 7.96 (d, J=4.7 Hz, 2H) 8.36 (d, J=4.9 Hz, 2H) 9.22 (s, 1H) 10.56 (s, 1H).

### E117 2-Amino-N-(1-hydroxy-1,3-dihydro-benzo[c][1,2]oxaborol-6-yl)-benzenesulfonamide

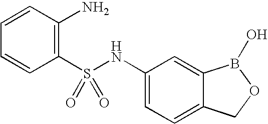

A suspension of N-(1-hydroxy-1,3-dihydro-benzo[c][1,2]oxaborol-6-yl)-2-nitro-benzenesulfonamide (0.85 g, 2.5 mmol), 10% Pd/C (200 mg), and abs. EtOH (150 mL) was shaken in a Parr apparatus at rt under an atmosphere of H₂ (50 psi) for 2.5 h. The mixture was filtered through Celite (washing with EtOH) and then a 0.2 μM filter. The filtrate was concentrated in vacuo at 40° C. and the residue was recrystallized (MeCN/H₂O) to give the title compound in two crops; yield 280 mg (37%). mp (crop 2) 143-144° C.; ¹H NMR (400 MHz, DMSO-d₆) δ (ppm): 10.17 (s, 1H), 9.20 (s, 1H), 7.48-7.45 (m, 2H), 7.25-7.14 (m, 3H), 6.73-6.71 (m, 1H), 6.53-6.49 (m, 1H), 5.98 (s, 2H), 4.86 (s, 2H); MS (ESI) m/z=303 (M−1, negative); HPLC purity: 95.57% (MaxPlot 200-400 nm), 95.01% (220 nm).

E118 3-Amino-N-(1-hydroxy-1,3-dihydro-benzo[c][1,2]oxaborol-6-yl)-benzenesulfonamide hydrochloride

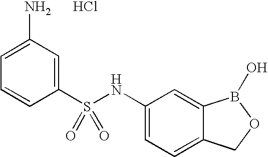

A suspension of N-(1-hydroxy-1,3-dihydro-benzo[c][1,2]oxaborol-6-yl)-3-nitro-benzenesulfonamide (430 mg, 1.40 mmol), 10% Pd/C (100 mg), and abs. EtOH (150 mL) was shaken in a Parr apparatus at rt under an atmosphere of H₂ (50 psi) for 2 h. The mixture was filtered through Celite° (washing with EtOH) and then a 0.2 μM filter. Purification by prep HPLC followed by lyophilization from 1 M HCl gave the title compound as a white solid; yield 105 mg (22%). ¹H NMR (400 MHz, DMSO-d₆) δ (ppm): 10.16 (s, 1H), 7.48-7.47 (m, 1H), 7.27-7.16 (m, 2H), 7.05 (s, 1H), 7.00-6.98 (m, 1H), 6.83-6.82 (m, 1H), 4.88 (s, 2H); MS (ESI) m/z=303 (M−1, negative); HPLC purity: 88.93% (MaxPlot 200-400 nm), 88.86% (220 nm).

### E119 4-Amino-N-(1-hydroxy-1,3-dihydro-benzo[c][1,2]oxaborol-6-yl)-benzenesulfonamide

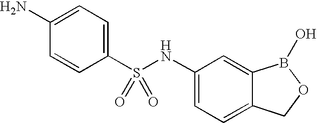

A mixture of N-(1-hydroxy-1,3-dihydro-benzo[c][1,2]oxaborol-6-yl)-4-nitro-benzenesulfonamide (0.6 g, 1.79 mmol), Pd/C (10% wet, 0.6 g) in methanol (50 mL) was placed under a hydrogen atmosphere at 50 psi for 0.5 h. The catalyst was filtered off through a pad of Celite° and the solvent was evaporated. The product was purified by column using 10% MeOHin dichloromethane, suspended in hot water and sonicated for 10 min to afford the target compound E119 (0.28 g, 51%) as a white solid after drying. mp 151-152° C.; ¹H NMR (400 MHz, DMSO-d₆) δ ppm 9.82 (s, 1H), 9.20 (s, 1H), 7.46 (s, 1H), 7.37 (d, J=9.0 Hz, 2H), 7.23 (d, J=8.2 Hz, 1H), 7.15 (d, J=8.2 Hz, 1H), 6.50 (d, J=9.0 Hz, 2H), 5.94 (s, 2H), 4.87 (s, 2H); ESI-MS m/z 305 (M+H, positive); HPLC purity: 94.19% (MaxPlot 200-400 nm), 93.45% (220 nm).

### E119 Alternate Synthesis

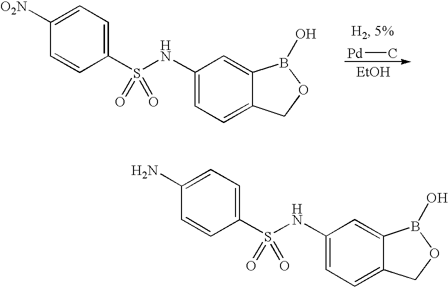

A 40 mL scintillation vial was charged with N-(1-hydroxy-1,3-dihydro-benzo[c][1,2]oxaborol-6-yl)-4-nitro-benzenesulfonamide (115 mg, 0.34 mmol, 1 eq) and EtOH (10 mL). The vial was purged with nitrogen (3×), then palladium on carbon (5% w/w, 20 mg) was added and the mixture was purged with hydrogen (3×) then held under an atmosphere of hydrogen. The mixture was stirred overnight, then the catalyst was removed by filtration and washed with EtOH (20 mL). The resulting solution was evaporated and the residue purified by silica gel chromatography (0-10% MeOH/DCM) to furnish Compound 019JMS062 as a white solid. LCMS (m/z) 305 [M+H]; ¹H NMR (400 MHz, DMSO- d₆) δ ppm 4.87 (s, 2H) 5.92 (s, 2H) 6.45-6.55 (m, 2H) 7.15 (dd, J=8.2, 2.1 Hz, 1H) 7.20-7.25 (m, 1H) 7.36 (d, J=8.8 Hz, 2H) 7.46 (d, J=2.0 Hz, 1H) 9.18 (s, 1H) 9.80 (s, 1H).

### E120 4-Dimethylamino-N-(1-hydroxy-1,3-dihydro-benzo[c][1,2]oxaborol-6-yl)-benzenesulfonamide

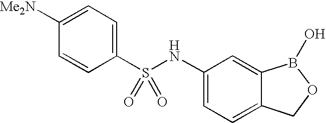

### 4-Dimethylamino-benzenesulfonic acid

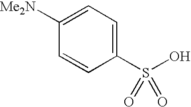

A mixture of N,N-dimethylaniline (2.5 g, 20 mmol) and bistrimethylsilyl sulfate (5.0 g, 20 mmol) was heated at 170° C. for 5 h. The mixture was allowed to cool to rt and the resulting solid was isolated by filtration and washed with Et₂O. The solid was then dissolved in H₂O, and the solution was concentrated in vacuo to give the title compound as a white solid: yield; 4.3 g (quant.). ¹H NMR (400 MHz, DMSO-d₆) δ (ppm): 7.59-7.57 (m, 2H), 7.10-7.02 (m, 2H), 3.07 (s, 6H); MS (ESI) m/z=202 (M+H, positive).

### 4-Dimethylamino-benzenesulfonyl chloride

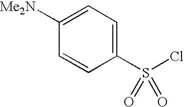

4-Dimethylamino-benzenesulfonic acid (4.3 g, 20 mmol) was added portionwise to a suspension of PCl₅ (5.0 g, 24 mmol) in CH₂Cl₂ (60 mL) at 0° C. The mixture was then allowed to warm to rt and was then stirred at rt for 3 h. The mixture was concentrated in vacuo and the residue was dissolved in Et₂O and H₂O. The layers were separated and the organic layer was dried (MgSO₄) and concentrated in vacuo to give the title compound as a yellow solid: yield; 1.95 g (42%). ¹H NMR (400 MHz, CDCl₃) δ (ppm): 7.84-7.82 (m, 2H), 6.77-6.75 (m, 2H), 3.10 (s, 6H).

### 4-Dimethylamino-N-(1-hydroxy-1, 3-dihydro-benzo[c][1,2]oxaborol-6-yl)-benzenesulfonamide

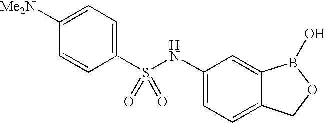

General Procedure 2: 6-Amino-3H-benzo[c][1,2]oxaborol-1-ol (200 mg, 1.34 mmol), 4-dimethylamino-benzenesulfonyl chloride (350 mg, 1.61 mmol), Si-pyridine (2.8 g, 4.0 mmol), and MeCN (20 mL) at rt O/N. Si-amine (0.8 g, 1.34 mmol) was added and the mixture was stirred at rt for 6 h. The mixture was then filtered and the resin was washed with MeCN. Water was added to the filtrate and the mixture was concentrated in vacuo at 40° C. until precipitate was observed. The solid was isolated by filtration to give the title compound as white needles: yield; 120 mg (27%). mp 153° C.; ¹H NMR (400 MHz, DMSO-d6) δ (ppm): 9.88 (s, 1H), 9.18 (s, 1H), 7.50-7.46 (m, 3H), 7.22-7.14 (m, 2H), 6.67-6.65 (m, 2H), 4.85 (s, 2H), 2.92 (s, 6H); MS (ESI) m/z=333 (M+H, positive); HPLC purity: 96.52% (MaxPlot 200-400 nm), 97.55% (220 nm).

### E 121 4-Formylamino-N-(1-hydroxy-1,3-dihydro-benzo[c][1,2]oxaborol-6-yl)-benzenesulfonamide

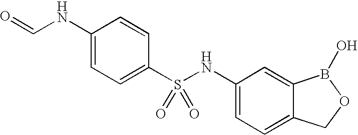

A suspension of 4-amino-N-(1-hydroxy-1,3-dihydro-benzo[c][1,2]oxaborol-6-yl)-benzenesulfonamide (0.40 g, 1.30 mmol) in formic acid (5.0 g, 130.0 mmol.) was heated at 100° C. (bath temp) for 16 h. The mixture was then cooled to rt and formic acid was removed under reduced pressure. The resulting residue was diluted with ethyl acetate (50 mL), and the solution was washed with saturated aq. NaHCO₃ solution, water, brine, dried over Na₂SO₄, decanted and concentrated under reduced pressure. The residue was dissolved in minimal amount of DCM and hexanes was added until the solution became cloudy. The precipitate was collected by filtration and was washed with hexanes. The solid was dried under vacuum providing 260 mg (60%) of the title compound. MS (ESI): m/z=331 (M−1, negative).

### E122 N-(1-Hydroxy-1,3-dihydro-benzo[c][1,2]oxaborole-6-yl)-4-methylamino-benzene sulfonamide

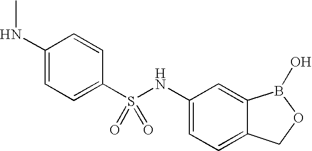

To a suspension of lithium aluminum hydride (30 mg, 0.70 mmol) in THF (15 mL) at −10° C. was added solution of 4-formylamino-N-(1-hydroxy-1,3-dihydro-benzo[c][1,2]oxaborol-6-yl)-benzenesulfonamide (0.20 g, 0.6 mmol) in THF (5 mL). The reaction mixture was allowed to warm to rt. After 3 h, the reaction mixture was cooled in an ice bath and saturated aq. NH₄Cl solution (5 mL) was added. The reaction mixture was diluted with ethyl acetate (100 mL), and the resulting organic layer was washed with water, brine and dried over Na₂SO₄, decanted and concentrated under reduced pressure. Purification was accomplished by preparative HPLC generating 70 mg (36%) of the title compound as pale yellow solid. ¹H NMR (400 MHz, DMSO-d6) δ (ppm): 9.82 (s, 1H), 9.19 (s, 1H), 7.47-7.42 (m, 3H), 7.23 (d, J=8.2 Hz, 1H), 7.16 (dd, J=8.2, 2.0 Hz, 1H), 6.51-6.48 (m, 3H), 4.86 (s, 2H), 2.66 (d, J=4.7 Hz, 3H); MS (ESI): m/z=319 (M+1, positive); HPLC purity: 96.91% (MaxPlot 200-400 nm), 97.47% (220 nm).

### E123 N-[4-(1-Hydroxy-1,3-dihydro-benzo[c][1,2]oxaborol-6-ylsulfamoyl)-phenyl]-acetamide

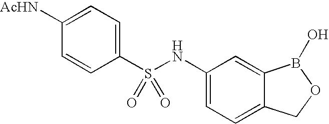

General Procedure 2: 6-amino-3H-benzo[c][1,2]oxaborol-1-ol (250 mg, 1.7 mmol), 4-acetamidobenzenesulfonyl chloride (466 mg, 2.0 mmol), NMM (0.74 mL, 6.8 mmol), and MeCN (5 mL) at rt O/N. The mixture was concentrated in vacuo. H₂O (5 mL) was added to the residue and the mixture acidified with 1 M HCl (5 drops). EtOAc (15 mL) was added and the mixture stirred until a clear biphasic solution was observed. The aqueous layer was loaded onto an Isolute HM-N column and left to stand for 10 min. The organic layer was then eluted through the column. The column was then further washed with EtOAc (20 mL). The organic fractions were concentrated in vacuo. Recrystallization from MeCN/H₂O gave give the title compound as a white solid: yield; 125 mg (21%). mp 226-227° C.; ¹H NMR (400 MHz, DMSO-d₆) δ (ppm): 10.28 (bs, 1H), 10.15 (bs, 1H), 9.25 (s, 1H), 7.71-7.62 (m, 4H), 7.43 (s, 1H), 7.24-7.22 (m, 1H), 7.19-7.16 (m, 1H), 4.88 (s, 2H), 2.03 (s, 3H); MS (ESI) m/z=345 (M−1, negative); HPLC purity: 95.04% (MaxPlot 200-400 nm), 95.35% (220 nm).

### E124 4-Hydroxyamino-N-(1-hydroxy-1,3-dihydro-benzo[c][1,2]oxaborol-6-yl)-benzenesulfonamide

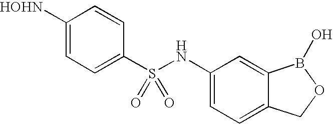

A mixture of N-(1-hydroxy-1,3-dihydro-benzo[c][1,2]oxaborol-6-yl)-4-nitro-benzene-sulfonamide (500 mg, 1.50 mmol), Zn powder (392 mg, 6.0 mmol) in sat NH₄Cl (20 mL) and CHCl₃ (20 mL) was stirred for 1 h. Solid particles were removed by filtration. After removal of organic solvent aqueous solution was then lyophilized. The crude material was then purified by preparative HPLC to afford the title compound as a white solid. Yield: 50 mg (10%). ¹H NMR (400 MHz, DMSO-d₆) δ ppm 9.19 (s, 1H), 8.93 (s, 1H), 8.62 (s, 1H), 7.58-7.41 (m, 3H), 7.27-7.21 (m, 1H), 7.18-7.10 (m, 1H), 6.78 (d, J=9.0 Hz, 2H), 4.87 (s, 2H); MS (ESI) m/z=319 (M−1, negative); HPLC purity: 96.27% (MaxPlot 200-400 nm), 96.67% (220 nm).

### E125 N-(1-Hydroxy-1,3-dihydro-benzo[c][1,2]oxaborol-6-yl)-4-methanesulfonylamino-benzenesulfonamide

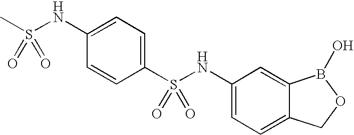

To a solution of 4-Amino-N-(1-hydroxy-1,3-dihydro-benzo[c][1,2]oxaborol-6-yl)-benzenesulfonamide (0.30 g, 0.986 mmol) in DCM (7 mL) was added pyridine (7 mL, 8.70 mmol) and the resulting mixture was cooled to 0° C. Methanesulfonyl chloride (0.08 mL, 1.08 mmol) was slowly added. After warming to room temperature and stirring overnight the reaction was heated to 60° C. for 5 h. The volatiles were removed in vacuo and the residue was treated with ethyl acetate and water. The organic layer was separated, washed with brine, dried over Na₂SO₄, filtered and concentrated in vacuo providing a yellow solid. Purification was accomplished by preparative HPLC (MeOH/water (0.1% formic acid) gradient) resulting in the isolation of 120 mg (32% yield) of the title compound as a white lyophilizate. ¹H NMR (400 MHz, DMSO-d₆) δ (ppm): 9.22 (s, 1H), 7.67 (d, J=8.6 Hz, 2H), 7.48 (d, J=1.9 Hz, 1H), 7.27-7.22 (m, 3H), 7.17 (dd, J=7.9, 1.9 Hz, 1H), 4.88 (s, 2H), 3.08 (s, 3H); MS (ESI) m/z=383 (M+1, positive); HPLC purity: 98.45% (MaxPlot 200-400 nm), 98.51% (220 nm).

### E126 N-(1-Hydroxy-1,3-dihydro-benzo[c][1,2]oxaborol-6-yl)-3-methanesulfonyl-benzenesulfonamide

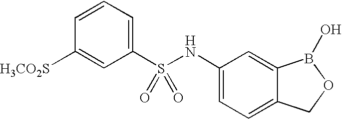

General Procedure 2: 6-amino-3H-benzo[c][1,2]oxaborol-1-ol (250 mg, 1.67 mmol), acetonitrile (7 mL), 3-methanesulfonyl-benzenesulfonyl chloride (513 mg, 2.01 mmol), N-methyl morpholine (678 mg, 6.70 mmol). Preparative HPLC purification using 0.1% formic acid/water and acetonitrile provided 49 mg (9%) of the title compound as a white solid. ¹H NMR (400 MHz, DMSO-d₆) δ ppm 9.19 (br. s., 1H), 8.20 (s, 1H), 8.13 (d, J=7.8 Hz, 1H), 8.0 (d, J=7.8 Hz, 1H), 7.85-7.75 (m, 1H), 7.46 (s, 1H), 7.25 (d, J=8.2 Hz, 1H), 7.13 (dd, J=8.2, 1.9 Hz, 1H), 4.86 (s, 2H), 3.30 (br. s., 3H); MS (ESI) m/z=365 (M−1, negative); HPLC purity: 97.15% (MaxPlot 200-400 nm), 97.72% (220 nm); Anal. Calcd for C₁₄H₁₄BNO₆S₂.0.33H₂O: C 45.09%; H 3.96%; N 3.75%.

### E127

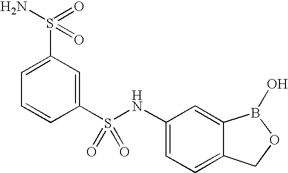

General Procedure 2: 6-amino-3H-benzo[c][1,2]oxaborol-1-ol (0.150 g, 1.01 mmol), MeCN (10 mL), pyridine (0.243 mL, 3.0 mmol), and 3-sulfamoyl-benzenesulfonyl chloride (0.245 g, 0.958 mmol). Purification: precipitation from acidic H₂O. E139 was isolated as an orange solid: yield 210 mg (60%). mp 199-201° C.; ¹H NMR (400 MHz, DMSO-d₆) δ (ppm): 10.49 (s, 1H), 9.22 (s, 1H), 8.22-8.21 (m, 1H), 8.02 (d, J=7.8 Hz, 1H), 7.90-7.88 (m, 1H), 7.75 (t, J=8.0 Hz, 1H), 7.59 (s, 2H), 7.49 (d, J=1.6 Hz, 1H), 7.28 (d, J=8.2 Hz, 1H), 7.18 (dd, J=8.2, 2.0 Hz, 1H), 4.89 (s, 2H); MS (ESI) m/z=367 (M−1, negative); HPLC purity: 96.29% (MaxPlot 200-400 nm), 96.26% (220 nm); Anal. Calcd for C₁₃H₁₃BN₂O₆S₂.0.1H₂O: C 42.20%; H 3.60%; N 7.57%. Found: C 41.94%; H 3.52%; N 7.77%.

### E128 Pyridine-4-sulfonic acid (1-hydroxy-1,3-dihydro-benzo[c][1,2]oxaborol-6-yl)-amide

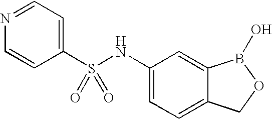

General Procedure 2: 6-amino-3H-benzo[c][1,2]oxaborol-1-ol, MeCN, K₂CO₃, and pyridine-4-sulfonyl chloride. The reaction is restarted with NMM to consume all the 6-amino-3H-benzo[c][1,2]oxaborol-1-ol. Purification: precipitation occurs from H₂O, flash chromatography (95:5 CH₂Cl₂/MeOH), then precipitation from H₂O. The title compound is isolated as a light yellow solid.

E129 6-Methoxy-pyridine-3-sulfonic acid (1-hydroxy-1,3-dihydro-benzo[c][1,2]oxaborol-6-yl)-amide

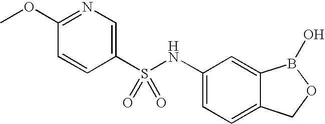

General Procedure 2: 6-amino-3H-benzo[c][1,2]oxaborol-1-ol.HCl (0.2 g, 1.08 mmol), MeCN (4 mL), pyridine (0.35 mL, 4.31 mmol), and 6-methoxy-pyridine-3-sulfonyl chloride (0.36 g, 1.73 mmol). Purification by flash chromatography (0-5% MeOH/CH₂Cl₂) gave the title compound as a white solid: yield 281 mg (81%). ¹H NMR (400 MHz, DMSO-d₆) δ ppm 10.30 (br. s., 1H), 9.23 (s, 1H), 8.48 (d, J=2.3 Hz, 1H), 7.94 (dd, J=8.8, 2.5 Hz, 1H), 7.49 (s, 1H), 7.34-7.23 (m, 1H), 7.23-7.11 (m, 1H), 6.96 (d, J=8.6 Hz, 1H), 4.89 (s, 2H), 3.88 (s, 3H); MS (ESI) m/z=319 (M−1, negative); HPLC purity: 98.30% (MaxPlot 200-400 nm), 98.17% (220 nm).

### E130 6-Hydroxy-pyridine-3-sulfonic acid (1-hydroxy-1,3-dihydro-benzo[c][1,2]oxaborol-6-yl)-amide

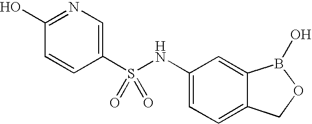

A stirred solution of 6-Methoxy-pyridine-3-sulfonic acid (1-hydroxy-1,3-dihydro-benzo[c][1,2]oxaborol-6-yl)-amide (0.2 g, 0.63 mmol) in 3N aqueous hydrochloric acid was heated at reflux for 3 h. After cooling to rt, the pH was raised to 6 by adding solid sodium bicarbonate and the aqueous layer extracted with ethyl acetate. The organic layer was washed with saturated aqueous sodium bicarbonate and brine, dried over sodium sulfate and concentrated in vacuo. Purification by preparative HPLC gave the title compound as a white solid: yield 51 mg (27%). ¹H NMR (400 MHz, DMSO-d₆) δ ppm 12.09 (br. s., 1H), 10.15 (br. s., 1H), 9.25 (s, 1H), 7.71 (br. s., 1H), 7.62-7.43 (m, 2H), 7.32 (d, J=8.2 Hz, 1H), 7.19 (dd, J=8.0, 1.8 Hz, 1H), 6.42 (d, J=9.8 Hz, 1H), 4.91 (s, 2H); MS (ESI) m/z=305 (M−1, negative); HPLC purity: 99.48% (MaxPlot 200-400 nm), 99.47% (220 nm).

### E131 5-Nitro-pyridine-2-sulfonic acid (1-hydroxy-1,3-dihydro-benzo[c][1,2]oxaborol-6-yl)-amide

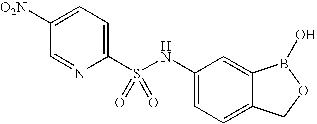

### 5-Nitro-pyridine-2-sulfonyl Chloride

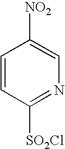

To an ice-cold solution of 5-nitro-pyridine-2-thiol (1.27 g, 8.13 mmol) in 1N aqueous HCl (25 mL) and acetic acid (5 mL) was vigorously bubbled chlorine (gas) for 15 min, followed by nitrogen for 5 min. the solid was collected by filtration, washed with cold 1N aqueous HCl and water and dried in vacuo: yield 842 mg (47%). ¹H NMR (400 MHz, CHLOROFORM-d) δ ppm 9.60 (d, J=2.0 Hz, 1H), 8.84 (dd, J=8.6, 2.3 Hz, 1H), 8.35 (d, J=8.6 Hz, 1H).

### 5-Nitro-pyridine-2-sulfonic acid (1-hydroxy-1,3-dihydro-benzo[c][1,2]oxaborol-6-yl)-amide

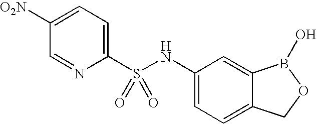

General Procedure 2: 6-amino-3H-benzo[c][1,2]oxaborol-1-ol (0.2 g, 1.34 mmol), MeCN (4 mL), pyridine (0.22 mL, 2.69 mmol), and 5-nitro-pyridine-2-sulfonyl chloride (0.3 g, 1.34 mmol). Purification by filtration from water and wash with water and ethyl acetate generated 380 mg (84%) of the title compound as an orange solid. mp 211-213° C.; ¹H NMR (400 MHz, DMSO-d₆) δ ppm 10.85 (s, 1H), 9.45 (d, J=2.2 Hz, 1H), 9.22 (s, 1H), 8.78 (dd, J=8.6, 2.5 Hz, 1H), 8.16 (d, J=8.6 Hz, 1H), 7.52 (s, 1H), 7.34-7.15 (m, 2H), 4.88 (s, 2H); MS (ESI) m/z=334 (M−1, negative); HPLC purity: 93.99% (MaxPlot 200-400 nm), 93.92% (220 nm).

### E132 5-Amino-pyridine-2-sulfonic acid (1-hydroxy-1,3-dihydro-benzo[c][1,2]oxaborol-6-yl)-amide; hydrochloride

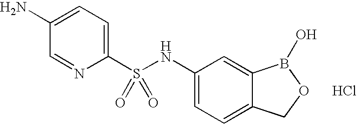

A mixture of 5-Nitro-pyridine-2-sulfonic acid (1-hydroxy-1,3-dihydro-benzo[c][1,2]oxaborol-6-yl)-amide (1.51 g, 4.51 mmol)) and 10% Pd on carbon (1.51 g, 1:1 w/w substrate to catalyst) in THF (30 mL) and methanol (135 mL) was shaken under an atmosphere of H₂ (40 psi) in a Parr shaker. Once the reaction was complete (30 min), the mixture was filtered through Celite. The filtrate was concentrated in vacuo and the residue dissolved in acetonitrile—water, washed with ethyl ether and lyophilized to provide the title compound as a yellow solid: yield 827 mg (60%). ¹H NMR (400 MHz, DMSO-d₆) δ ppm 10.10 (s, 1H), 9.17 (br. s., 1H), 7.90 (d, J=2.5 Hz, 1H), 7.56 (d, J=8.6 Hz, 1H), 7.47 (s, 1H), 7.27-7.13 (m, 2H), 6.89 (dd, J=8.6, 2.5 Hz, 1H), 6.17 (br. s., 2H), 4.86 (s, 2H); MS (ESI): m/z=304 (M−1, negative); HPLC purity: 95.56% (MaxPlot 200-400 nm), 95.55% (220 nm).

### E133 6-Amino-pyridine-3-sulfonic acid (1-hydroxy-1,3-dihydro-benzo[c][1,2]oxaborol-6-yl)-amide hydrochloride

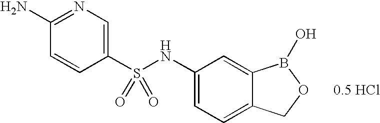

### 6-Amino-pyridine-3-sulfonyl chloride

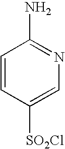

Pyridin-2-ylamine (2 g, 21.25 mmol) was added slowly to ice-cold chlorosulfonic acid (14 mL, 212.5 mmol) in a sealable flask. Thionyl chloride (6.2 mL, 85 mmol) was added dropwise and the flask was sealed. The mixture was heated at 80° C. for 2.5 h and at 150° C. for 16 h. After cooling to rt, the mixture was cautiously poured on crushed ice and the resulting precipitate filtered off. The filtrate was extracted with ethyl acetate (3 times) and the combined organic layers washed brine, dried over sodium sulfate and dried in vacuo to give the title compound as a white solid: yield 870 mg (21%). ¹H NMR (400 MHz, CHLOROFORM-d) δ ppm 8.70 (d, J=1.9 Hz, 1H), 7.96 (dd, J=9.0, 2.4 Hz, 1H), 6.57 (d, J=8.9 Hz, 1H), 5.29 (br. s., 2H); MS (ESI): m/z=191 (M−1, negative).

### 6-Amino-pyridine-3-sulfonic acid (1-hydroxy-1,3-dihydro-benzo[c][1,2]oxaborol-6-yl)-amide; hydrochloride

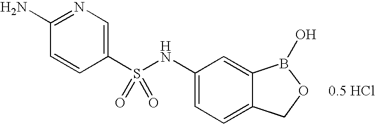

General Procedure 2: 6-amino-3H-benzo[c][1,2]oxaborol-1-ol (0.32 g, 2.15 mmol), MeCN (7 mL), pyridine (0.35 mL, 4.31 mmol), and 6-amino-pyridine-3-sulfonyl chloride (0.42 g, 2.15 mmol). Purification by preparative HPLC generated 260 mg (40%) of the title compound as a pale yellow solid after lyophilization. ¹H NMR (400 MHz, DMSO-d₆) δ ppm 10.04 (s, 1H), 9.22 (br. s., 1H), 8.19 (d, J=2.2 Hz, 1H), 7.62 (dd, J=9.0, 2.4 Hz, 1H), 7.49 (d, J=1.6 Hz, 1H), 7.28 (d, J=8.2 Hz, 1H), 7.17 (dd, J=8.2, 1.9 Hz, 1H), 7.10 (br. s., 2H), 6.50 (d, J=8.9 Hz, 1H), 4.89 (s, 2H); MS (ESI) m/z=306 (M+1, positive); HPLC purity: 99.85% (MaxPlot 200-400 nm), 99.55% (220 nm); Anal. Calcd for C₁₂H₁₂BN₃O₄S.0.5 HCl: C 44.57%; H 3.90%; N 13.00%. Found: C 44.97%; H 4.19%; N 12.72%.

### E134 5-(1-Hydroxy-1,3-dihydro-benzo[c][1,2]oxaborol-6-ylsulfamoyl)-furan-2-carboxylic acid methyl ester

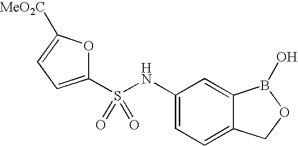

General Procedure 2: 6-amino-3H-benzo[c][1,2]oxaborol-1-ol.HCl (0.2 g, 1.08 mmol), MeCN (4 mL), pyridine (0.35 mL, 4.32 mmol), and 5-chlorosulfonyl-furan-2-carboxylic acid methyl ester (0.29 g, 1.3 mmol). Purification by flash chromatography (0-5% MeOH/CH₂Cl₂) gave the title compound as a white solid: yield 284 mg (78%). ¹H NMR (400 MHz, DMSO-d₆) δ ppm 10.93 (br. s., 1H), 9.25 (s, 1H), 7.51 (d, J=1.6 Hz, 1H), 7.43-7.28 (m, 2H), 7.27-7.15 (m, 2H), 4.92 (s, 2H), 3.84 (s, 3H).

### E135 5-Hydroxymethyl-furan-2-sulfonic acid (1-hydroxy-1,3-dihydro-benzo[c][1,2]oxaborol-6-yl)-amide

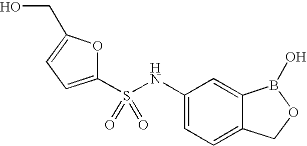

A 1M solution of lithium borohydride (1.04 mL, 2.08 mmol) was added dropwise to an ice-cold solution of 5-(1-hydroxy-1,3-dihydro-benzo[c][1,2]oxaborol-6-ylsulfamoyl)-furan-2-carboxylic acid methyl ester (0.23 g, 0.69 mmol) in anhydrous THF (5 mL) and methanol (0.1 mL) and the mixture was stirred at rt for 3 h. The mixture was then cooled to 0° C. and acidified to pH 6 with 3N aqueous HCl. The mixture was extracted with ethyl acetate and the organic layer washed with brine, dried over sodium sulfate and concentrated in vacuo to give the title compound as a white solid: yield 153 mg (71%). ¹H NMR (400 MHz, DMSO-d₆) δ ppm 10.61 (s, 1H), 9.25 (br. s., 1H), 7.53 (s, 1H), 7.30 (d, J=8.3 Hz, 1H), 7.20 (dd, J=8.3, 1.9 Hz, 1H), 7.04 (d, J=3.5 Hz, 1H), 6.42 (d, J=3.5 Hz, 1H), 5.46 (br. s., 1H), 4.91 (s, 2H), 4.40 (s, 2H); MS (ESI) m/z=308 (M−1, negative); HPLC purity: 95.43% (MaxPlot 200-400 nm), 95.15% (220 nm); Anal. Calcd for C₁₂H₁₂BNO₆.2H₂O : C 41.76%, H 4.67%; N 4.06%. Found: C 41.50%; H 4.47%; N 4.33%.

### E136 1H-Pyrazole-4-sulfonic acid (1-hydroxy-1,3-dihydro-benzo[c][1,2]oxaborol-6-yl)-amide

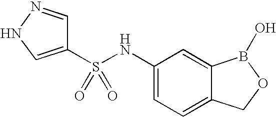

General Procedure 2: 6-amino-3H-benzo[c][1,2]oxaborol-1-ol (0.200 g, 1.07 mmol), MeCN (10 mL), NMM (0.23 mL, 2.14 mmol), 1H-pyrazole-4-sulfonyl chloride (0.189 g, 1.07 mmol). Purification: preparative HPLC. E136 was isolated as white solid; yield 50 mg (16%). ¹H NMR [400 MHz, METHANOL-d₄+Conc HCl (1 drop)] δ ppm 7.89 (br.s, 2H), 7.45 (d, J=2.0 Hz, 1H), 7.37-7.20 (m, 2H), 4.98 (s, 2H); MS (ESI) m/z=280 (M+1, positive); HPLC purity: 97.48% (MaxPlot 200-400 nm), 98.72% (220 nm).

### E137 1H-Imidazole-4-sulfonic acid (1-hydroxy-1,3-dihydro-benzo[c][1,2]oxaborol-6-yl)-amide

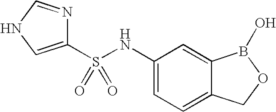

General Procedure 2: 6-amino-3H-benzo[c][1,2]oxaborol-1-ol (0.500 g, 2.69 mmol), MeCN (20 mL), NMM (0.88 mL, 8.07 mmol), and 1H-imidazole-4-sulfonyl chloride (0.493 g, 2.95 mmol). Purification: Recrystallization from hot water. E137 was isolated as orange solid; yield 50 mg (16%). m.p. 195-196° C. ¹H), NMR (400 MHz, DMSO-d₆) δ ppm 12.67 (br. s, 1H), 10.15 (s, 1H), 9.20 (s, 1H), 7.79 (s, 1H), 7.71 (s, 1H), 7.51 (s, 1H), 7.23 (s, 2H), 4.87 (s, 2H); MS (ESI) m/z=278 (M−1, negative); HPLC purity: 96.57% (MaxPlot 200-400 nm), 95.35% (220 nm).

### E138 N-[5-(1-Hydroxy-1,3-dihydro-benzo[c][1,2]oxaborol-6-ylsulfamoyl)-thiazol-2-yl]-acetamide

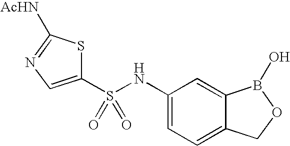

General Procedure 2: 6-amino-3H-benzo[c][1,2]oxaborol-1-ol (0.216 g, 1.07 mmol), pyridine (10 mL), and 2-acetylamino-thiazole-5-sulfonyl chloride (0.284 g, 1.17 mmol), 60° C. for 1.5 h. Purification: Recrystallization from hot water. E138 was isolated as orange solid; yield 120 mg (29%). mp. 235-236° C. ¹H NMR (400 MHz, DMSO-d₆) δ ppm 12.61 (s, 1H), 10.41 (s, 1H), 9.25 (s, 1H), 7.80 (s, 1H), 7.53 (d, J=2.0 Hz, 1H), 7.37-7.27 (m, 1H), 7.28-7.18 (m, 1H), 4.91 (s, 2H), 2.16 (s, 3H); MS (ESI) m/z=352 (M−1, negative); HPLC purity: 91.99% (MaxPlot 200-400 nm), 92.41% (220 nm).

### E139 2-Amino-thiazole-5-sulfonic acid (1-hydroxy-1,3-dihydro-benzo[c][1,2]oxaborol-6-yl)-amide

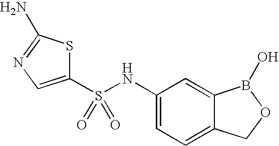

To a solution of N-[5-(1-hydroxy-1,3-dihydro-benzo[c][1,2]oxaborol-6-ylsulfamoyl)-thiazol-2-yl]-acetamide (90 mg, 0.25 mmol) in THF (5 mL) was added HCl (5 mL, 10%) and the reaction mixture was heated to 60° C. for 4 h. After removing the solvent in vacuo, purification was accomplished by preparative HPLC to afford 28 mg (36%) of the title compound as a white solid. ¹H NMR (400 MHz, DMSO-d₆) δ ppm 10.15 (s, 1H), 7.79 (s, 2H), 7.55 (d, J=1.6 Hz, 1H), 7.36-7.29 (m, 2H), 7.25-7.20 (m, 1H), 4.92 (s, 2H); MS (ESI) m/z=310 (M−1, negative); HPLC purity: 95.14% (MaxPlot 200-400 nm), 95.44% (220 nm).

### E140 1H-[1,2,4]Triazole-3-sulfonic acid (1-hydroxy-1,3-dihydro-benzo[c][1,2]oxaborol-6-yl)-amide

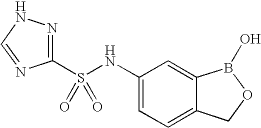

General Procedure 2: 6-amino-3H-benzo[c][1,2]oxaborol-1-ol (0.200 g, 1.07 mmol), MeCN (5 mL), NMM (0.35 mL, 3.21 mmol), 1H-[1,2,4]triazole-3-sulfonyl chloride (0.197 g, 1.17 mmol). Purification: Recrystallization from hot water. E140 was isolated as orange solid; yield 170 mg (56%). m.p. >300° C. (dec.). ¹H NMR (400 MHz, DMSO-d₆) δ ppm 10.69 (br. s., 1H), 9.23 (s, 1H), 8.75 (s, 1H), 7.53 (s, 1H), 7.30-7.25 (m, 2H), 4.90 (s, 2H); MS (ESI) m/z=279 (M−1, negative); HPLC purity: 98.32% (MaxPlot 200-400 nm), 98.94% (220 nm).

### E141 Cyclopropanesulfonic acid (1-hydroxy-1,3-dihydro-benzo[c][1,2]oxaborol-6-yl)-amide

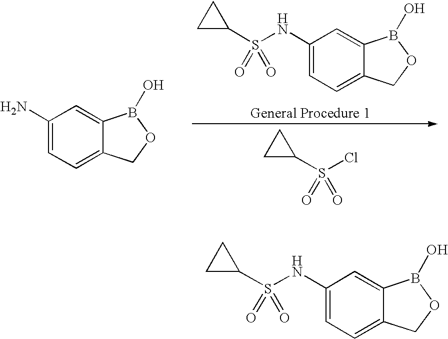

General Procedure 1: 6-amino-3H-benzo[c][1,2]oxaborol-1-ol (0.865 g, 5.81 mmol), MeCN (30 mL), NMM (2.55 mL, 23.2 mmol), and cyclopropanesulfonyl chloride (0.898 g, 6.39 mmol). Purification: flash chromatography (95:5 CH₂Cl₂/MeOH, sample absorbed to 14 g SiO₂ with CH₂Cl₂/MeOH) then trituration with EtOAc. E141 was isolated as a light yellow solid: yield 0.373 g (25%). mp 177-181° C.; ¹H NMR (400 MHz, DMSO-d₆) δ (ppm): 9.64 (bs, 1H), 9.22 (s, 1H), 7.62 (s, 1H), 7.34 (d, J=7.8 Hz, 1H), 7.31 (dd, J=8.2, 1.6 Hz, 1H), 4.93 (s, 2H), 2.54-2.49 (m, 1H), 0.92-0.87 (m, 4H); MS (ESI): m/z=252 (M−1, negative); HPLC purity: 99.25% (MaxPlot 200-400 nm), 99.05% (220 nm); Anal. Calcd for C₁₀H₁₂BNO₄S.0.5H₂O: C 46.63%; H 4.89%; N 5.44%. Found: C 46.51%; H 4.71%; N 5.52%.

### E142 4-(1-Hydroxy-1,3-dihydro-benzo[c][1,2]oxaborol-6-ylsulfamoyl)-piperidine-1-carboxylic acid benzyl ester

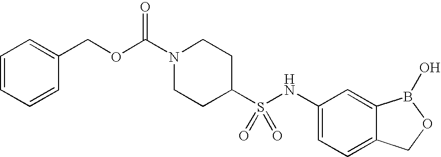

To a solution of 6-amino-3H-benzo[c][1,2]oxaborol-1-ol (HCl salt, 0.145 g, 0.786 mmol) in pyridine (7 mL, 8.70 mmol) cooled to 0° C. was added 4-chlorosulfonyl-piperidine-1-carboxylic acid benzyl ester (0.25 g, 0.786 mmol). After warming to room temperature and stirring overnight the reaction was heated to 70° C. for 6 h. The volatiles were removed in vacuo and the residue was treated with ethyl acetate and water. The organic layer was separated, washed with brine, dried over Na₂SO₄, filtered and concentrated in vacuo providing a brown oil. Purification was accomplished by preparative HPLC (MeOH/water (0.1% formic acid) gradient) resulting in the isolation of 140 mg (41% yield) of the title compound as a white lyophilizate. ¹HNMR (400 MHz, DMSO-d₆) δ (ppm): 9.92 (s, 1H), 9.26 (s, 1H), 7.62 (s, 1H), 7.39-7.31 (m, 7H), 5.07 (s, 2H), 4.94 (s, 2H), 4.04 (d, J=12.1 Hz, 2H), 3.29-3.17 (m, 1H), 2.99-2.70 (m, 2H), 1.98 (d, J=11.7 Hz, 2H), 1.55-1.47 (m, 2H); MS (ESI) m/z=429 (M−1, negative); HPLC purity: 99.54% (MaxPlot 200-400 nm), 99.62% (220 nm).

### E143 Piperidine-4-sulfonic acid (1-hydroxy-1,3-dihydro-benzo[c][1,2]oxaborol-6-yl)-amide; hydrochloride salt

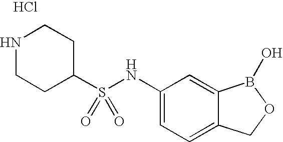

To a solution of 4-(1-Hydroxy-1,3-dihydro-benzo[c][1,2]oxaborol-6-ylsulfamoyl)-piperidine-1-carboxylic acid benzyl ester (0.10 g, 0.23 mmol) in methanol (4 mL) was added Pd/C (10% by wt, wet, 0.09 g) and a balloon filled with hydrogen. After overnight, filter through Celite® and rinse with methanol followed by chloroform/methanol (1:1) mixture and concentrate in vacuo. Purification was accomplished by preparative HPLC (MeOH/water (0.1% formic acid) gradient) followed by treatment of the lyophilizate in methanol (1.5 mL) with 1M HCl in ether (0.169 mL, 0.169 mmol) and concentration in vacuo and lyophilization from water generating 40 mg (52% yield) of the title compound. ¹H NMR (400 MHz, DMSO-d₆) δ (ppm): 10.03 (br.s, 1H), 9.28 (s, 1H), 7.64 (s, 1H), 7.39-7.33 (m, 2H), 4.95 (s, 2H), 3.42-3.34 (m, 2H), 2.89 (t, J=12.5 Hz, 2H), 2.51-2.50 (m, 1H), 2.11 (d, J=12.9 Hz, 2H), 1.90-1.82 (m, 2H); MS (ESI) m/z=297 (M+1, positive); HPLC purity: 98.34% (MaxPlot 200-400 nm), 99.15% (220 nm).

### E144 N-(1-Hydroxy-3-nitromethyl-1,3-dihydro-benzo[c][1,2]oxaborol-6-yl)-4-nitro-benzenesulfonamide

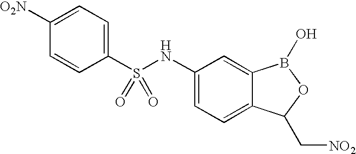

General Procedure 2: 6-Amino-3-nitromethyl-3H-benzo[c][1,2]oxaborol-1-ol hydrochloride (0.75 g, 3.1 mmol), 4-nitrobenzenesulfonyl chloride (0.82 g, 3.7 mmol), pyridine (1.0 mL, 12 mmol), and MeCN (10 mL) at rt O/N. Mixture was concentrated in vacuo at 40° C. The residue was dissolved in EtOAc and loaded onto a pre-column (SiO₂, 12 g). Purification by Biotage (10-100% EtOAc/CH₂Cl₂) gave the title compound as a yellow oil which solidified on standing under high vacuum: yield; 510 mg (42%). ¹H NMR (400 MHz, DMSO-d₆) δ (ppm): 10.73 (s, 1H), 9.56 (s, 1H), 8.38-8.36 (m, 2H), 8.01-8.00 (m, 2H), 7.50-7.49 (m, 1H), 7.45-7.43 (m, 1H), 7.24-7.21 (m, 1H), 5.68 (dd, J=8.9, 2.5 Hz, 1H), 5.25 (dd, J=13.6, 2.5 Hz, 1H), 5.25 (dd, J=13.6, 8.9 Hz, 1H); MS (ESI) m/z=392 (M−1, negative).

### E145 4-Amino-N-(3-aminomethyl-1-hydroxy-1,3-dihydro-benzo[c][1,2]oxaborol-6-yl)-benzenesulfonamide dihydrochloride

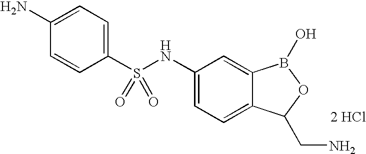

A mixture of N-(1-hydroxy-3-nitromethyl-1,3-dihydro-benzo[c][1,2]oxaborol-6-yl)-4-nitrobenzenesulfonamide (200 mg, 0.51 mmol), Raney nickel (100 mg), conc. NH₄OH (1.0 mL), H₂O (10 mL), and MeOH (5 mL) was shaken in a Parr apparatus under an atmosphere of H₂ (50 psi) at rt O/N. The mixture was filtered through celite and the filtrate was concentrated in vacuo. The residue was purified by prep HPLC (MeOH/0.1% aq TFA) and then lyophilized from 1 M HCl to give the title compound as a yellow solid: yield; 11 mg (5%). ¹H NMR (400 MHz, DMSO-d₆δ (ppm): 10.00 (s, 1H), 9.56 (bs, 1H), 8.00 (bs, 3H), 7.56-7.55 (m, 1H), 7.43-7.41 (m, 2H), 7.36-7.34 (m, 1H), 7.20-7.19 (m, 1H), 6.54-6.51 (m, 2H), 5.22-5.20 (m, 1H), 3.40-3.30 (hidden, 1H), 2.74-2.67 (m, 1H); MS (ESI) m/z=334 (M+H, positive); HPLC purity: 88.52% (MaxPlot 200-400 nm), 86.81% (220 nm).

### E146 N-(3-Aminomethyl-1-hydroxy-1,3-dihydro-benzo[c][1,2]oxaborol-6-yl)-3-difluoromethoxy-benzenesulfonamide, TFA salt

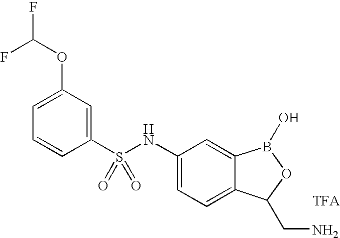

A mixture of 6-amino-3-aminomethyl-3H-benzo[c][1,2]oxaborol-1-ol dihydrochloride (250 mg, 0.52 mmol), silica bound diethylamine (2.0 g, 1.25 mmol g⁻¹, 2.6 mmol), and MeOH (10 mL) was stirred at rt for 30 min. The mixture was then concentrated in vacuo at 40° C. and then further dried on a high vac at rt O/N. Boc₂O (113 mg, 0.52 mmol) and THF (10 mL) were added and the resulting mixture was stirred vigorously at rt for 5 h. 3-(Difluoromethoxy)benzene sulfonyl chloride (378 mg, 1.56 mmol) was added and the mixture was stirred at rt O/N. The mixture was concentrated in vacuo at 30° C. The silica was placed in a Dasi dry loading unit. Purification using a Teledyne cyano column (50 g), running a gradient of hexane to CH₂Cl₂, then CH₂Cl₂ to MeOH resulted in the isolation of a mixture of [6-(3-difluoromethoxy-benzenesulonylamino)-1-hydroxy-1,3-dihydro-benzo[c][1,2]oxaborol-3-ylmethyl]-carbamic acid tert-butyl ester and 3-difluoromethoxy-benzenesulfonic acid. This mixture was dissolved in 4 M HCl/dioxane (10 mL) and the resulting solution was stirred at rt O/N. The mixture was concentrated in vacuo at 40° C. and the residue was purified by prep HPLC: gradient 5 to 10% MeCN/0.1% aq TFA over 2 min, then 10 to 90% MeCN/0.1% aq TFA over 15 min. The fractions were concentrated in vacuo at 40° C. to remove the organics and then freeze dried to give the title compound as a yellow solid: yield 13 mg (5%). ¹H NMR (400 MHz, DMSO-d₆): δ 10.54 (s, 1H), 9.61 (s, 1H), 8.00 (bs, 3H), 7.64-7.62 (m, 2H), 7.59-7.58 (m, 1H), 7.51 (s, 1H), 7.47-7.41 (m, 2H, 7.29 (s, 1H), 7.22-7.20 (m, 1H), 5.23-5.22 (m, 1H), 2.73-2.67 (m, 1H); ¹⁹F NMR (376 MHz, DMSO-d₆): δ −74.13 (s), −83.32 (d); MS (ESI) m/z=385 (M+1, positive); HPLC purity: 89.81% (MaxPlot 200-400 nm), 91.75% (220 nm).

### E147 5-Oxazol-5-yl-thiophene-2-sulfonic acid (3-aminomethyl-1-hydroxy-1,3-dihydro-benzo[c][1,2]oxaborol-6-yl)-amide, hydrochloride

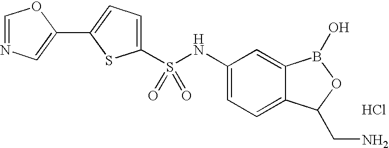

A mixture of 6-amino-3-aminomethyl-3H-benzo[c][1,2]oxaborol-1-ol dihydrochloride (250 mg, 0.52 mmol), silica bound diethylamine (2.0 g, 1.25 mmol g⁻¹, 2.6 mmol), and MeOH (10 mL) was stirred at rt for 15 min. The mixture was then concentrated in vacuo at 30° C. and then further dried under high vac at rt O/N. Boc₂O (113 mg, 0.52 mmol) and THF (10 mL) were added and the resulting mixture was stirred vigorously at rt for 8.5 h. 5-(1,3-Oxazol-5-yl)-2-thiophene sulfonyl chloride (0.39 g, 1.56 mmol) was added and the mixture was stirred at rt O/N. The mixture was concentrated in vacuo at 30° C. The silica was placed in a Dasi dry loading unit. Purification using a Teledyne cyano column (50 g), running a gradient of hexane to CH₂Cl₂, then CH₂Cl₂ to MeOH resulted in the isolation of a mixture of [1-hydroxy-6-(5-oxazol-5-yl-thiophene-2-sulfonylamino)-1,3-dihydro-benzo[c][1,2]oxaborol-3-ylmethyl]-carbamic acid tert-butyl ester and 5-oxazol-5-yl-thiophene-2-sulfonic acid. This mixture was dissolved in 4 M HCl/dioxane (10 mL) and the resulting solution was stirred at rt O/N. The mixture was concentrated in vacuo at 40° C. and the residue was purified by prep HPLC: gradient 5 to 10% MeCN/0.1% aq TFA over 2 min, then 10 to 90% MeCN/0.1% aq TFA over 15 min. The fractions were concentrated in vacuo at 40° C. to remove the organics, 1 M HCl was added and then the solution was freeze dried to give the title compound as a yellow solid: yield 6 mg (3%). ¹H NMR (400 MHz, DMSO-d₆): δ 10.79 (s, 1H), 8.52 (s, 1H), 8.20 (bs, 3H), 7.74 (s, 1H), 7.75-7.74 (m, 1H), 7.66-7.59 (m, 1H), 7.49-7.45 (m, 2H), 7.30-7.28 (m, 1H), 5.30-5.29 (m, 1H), 3.48-3.39 (m, 1H), 2.78-2.71 (m, 1H); MS (ESI) m/z=392 (M+1, positive); HPLC purity: 88.98% (MaxPlot 200-400 nm), 91.03% (220 nm).

### E148 N-((1-Hydroxy-6-(phenylsulfonamido)-1,3-dihydrobenzo[c][1,2]oxaborol-3-yl)methyl-4-methylpentanamide

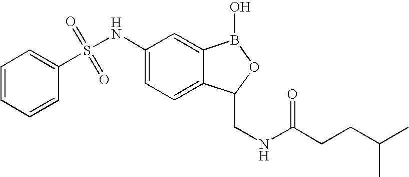

### Step 1. N-((1-Hydroxy-1,3-dihydrobenzo[c][1,2]oxaborol-3-yl)methyl)-4-methylpentanamide

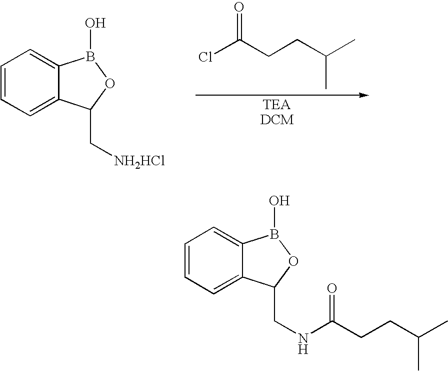

To a suspension of 3-(aminomethyl)benzo[c][1,2]oxaborol-1(3H)-ol (8.59 g, 43.15 mmol) in 145 ml of DCM was added triethylamine (18 ml, 129.5 mmol), cooled to 0° C., then slowly added 4-methylvaleryl chloride (5.81 g, 43.15 mmol). The reaction mixture was slowly warmed up to room temperature and stirred for additional 1.5 hours. It was filtered and the filtrate was collected and dried. The crude residue was re-suspended in CAN, filtered and dried to give 8.23 g off-white powder. 1 g of this powder was purified by flash chromatography to give 0.86 g product as white powder. MS (ESI) m/z 260 [M−H]⁻

### Step 2. N-((1-Hydroxy-6-nitro-1,3-dihydrobenzo[c][1,2]oxaborol-3-yl)methyl)-4-methylpentanamide

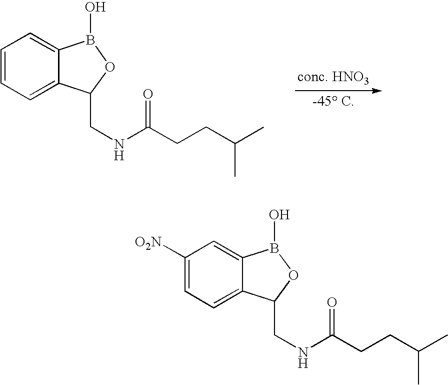

N-((1-hydroxy-1,3-dihydrobenzo[c][1,2]oxaborol-3-yl)methyl)-4-methylpentanamide (1.08 g, 4.14 mmol) was added to 5 ml concentrated HNO₃ at −50° C. and stirred for 2 hours. The mixture was then poured over crushed ice and extracted with ethyl acetate. Combined organic layer was washed with brine, concentrated and purified by column to get yellowish oil (0.96 g). MS (ESI) m/z 593 [2*M−18−H]⁺

### Step 3. N-((6-Amino-1-hydroxy-1,3-dihydrobenzo[c][1,2]oxaborol-3-yl)methyl)-4-methylpentanamide

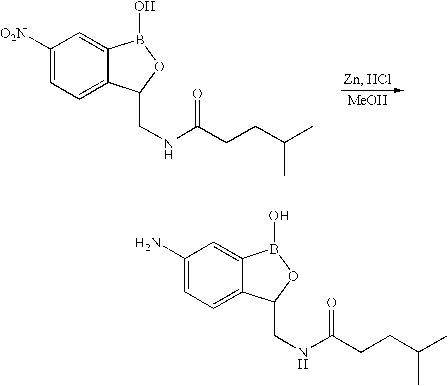

N-((1-hydroxy-6-nitro-1,3-dihydrobenzo[c][1,2]oxaborol-3-yl)methyl)-4-methylpentanamide (0.96g, 3.14 mmol) was dissolved in 80 ml MeOH and 1N HCl (62 ml, 62.8 mmol) then Zn powder (2.05 g, 31.4 mmol) were added. After 1 hour, 100 ml saturated sodium bicarbonate and 150 ml ethyl acetate were added. The mixture was stirred vigorously and an intense precipitate was produced. This was filtered through Celite and the Celite was rinsed with more ethyl acetate. The organic layer of the combined filtrate was separated, rinsed with more saturated sodium bicarbonate, brine, dried over Na₂SO₄, filtered and evaporated. The crude residue was purified by flash chromatography to get 0.44 g off-white solid. MS (ESI) m/z 533 [2*M−18−H]⁺

### Step 4. N-(1-Hydroxy-6-(phenylsulfonamido)-1,3-dihydrobenzo[c][1,2]oxaborol-3-yl)methyl)-4-methylpentanamide

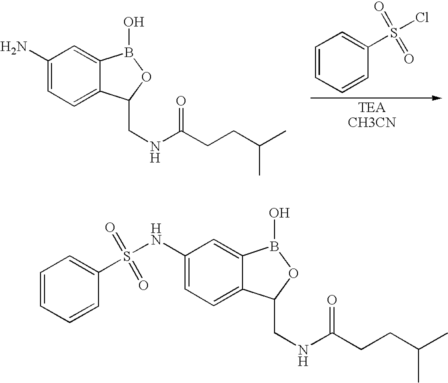

N-((6-amino-1-hydroxy-1,3-dihydrobenzo[c][1,2]oxaborol-3-yl)methyl)-4-methylpentanamide (0.44 g, 1.59 mmol, 1 eq) was dissolved in 10 ml ACN. Triethylamine (0.22 ml, 1.59 mmol, 1 eq) followed by benzenesulfonyl chloride (0.2 ml, 1.59 mmol, 1 eq) were added and the mixture was stirred for 2 hours at room temperature. The solvent was then evaporated by reduced pressure and the residue was dissolved in ethyl acetate, washed with saturated sodium bicarbonate, brine, dried over Na₂SO₄, filtered and evaporated. The crude residue was purified by flash chromatography to get 120 mg title compound as white powder. ¹H NMR (DMSO-d₆, 300 MHz) δ ppm 10.3 (s, 1H) 9.25 (s, 1H) 7.92 (t, J=5.4 Hz, 1H) 7.74 (d, J=5.3 Hz, 2H) 7.61-7.46 (m, 3H) 7.22 (d, J=8.1 Hz, 1H) 7.12 (d, J=8.3 Hz, 1H) 5.03-5.00 (m, 1H) 3.42-3.36 (m, 1H) 3.18-3.16 (m, 1H) 2.00 (t, J=7.2 Hz, 2H) 1.34-1.22 (m, 3H) 0.79 (d, J=11.1 Hz, 6H) MS (ESI) m/z 415 [M−H]⁺.

### E149 7-Phenoxy-3,4-dihydro-benzo[c][1,2]oxaborinin-1-ol

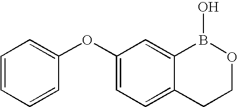

### Step 1. 2-Bromo-4-phenoxy-benzaldehyde

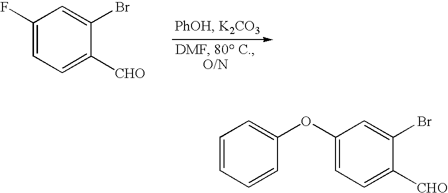

To a solution of 2-bromo-4-fluoro-benzaldehyde (10.0 g, 49.0 mmol) in DMF (60 mL) was added potassium carbonate (10.25 g, 73.8 mmol), followed by addition of phenol (4.6 g, 49.0 mmol). The resulting mixture was heated at 100° C. for 7 h. The reaction mixture was diluted with EtOAc and washed with water, brine, dried over Na₂SO₄, and concentrated under reduced pressure to give 2-bromo-4-phenoxy-benzaldehyde as an off white solid, which was used for the next step without further purification. ¹H NMR (400 MHz, CDCl₃) δ 10.25 (s, 1H), 7.90 (d, J=8.8 Hz, 1H), 7.46-7.41 (m, 2H), 7.25 (m, 1H), 7.16 (d, J=2.0 Hz, 1H), 7.17-7.07 (m, 2H), 6.98 (m, 1H).

### Step 2. (2-Bromo-4-phenoxy-phenyl)-acetaldehyde

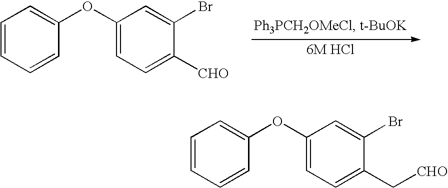

To a solution of (methoxymethyl)triphenylphosphonium chloride (1.05 g, 3.07 mmol) in DMSO (10 mL) was added potassium tert-butoxide (0.3 g, 2.7 mmol) and stirred at room temperature for 1 h. 2-Bromo-4-phenoxy-benzaldehyde in 10 mL of DMSO was added dropwise to the reaction mixture and stirred at room temperature overnight. The reaction mixture was quenched with saturated ammonium chloride, extracted with EtOAc, washed with water, brine, dried over Na₂SO₄, and concentrated under reduced pressure to give crude product, which was purified by column chromatography (silica gel, 5% yield EtOAc in hexane) to give 0.35 g of white solid, which was dissolved in 10 mL of THF and 2 mL of 6M HCl. The reaction mixture was heated to reflux for 6 h, extracted with EtOAc, washed with water, brine, dried over Na₂SO₄, and concentrated under reduced pressure to give (2-bromo-4-phenoxy-phenyl)-acetaldehyde (0.35 g, 66% yield) as a colorless oil. ¹H NMR (400 MHz, CDCl₃) δ 9.76 (t, J=2.4 Hz, 1H), 7.37 (t, J=5.0, 7.2 Hz, 2H), 7.25 (m, 2H), 7.18 (s, 1H), 7.02 (dd, J=8.8, 1.2 Hz, 2H), 6.95 (dd, J=8.4, 2.4 Hz, 1H), 3.8 (d, J=1.6 Hz, 2H).

### Step 3. 2-(2-Bromo-4-phenoxy-phenyl)-ethanol

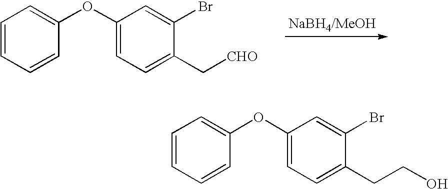

To a solution of (2-bromo-4-phenoxy-phenyl)-acetaldehyde (0.35 g, 1.20 mmol) in methanol (10 mL) was added sodium borohydride (0.055 g, 1.44 mmol) at 0° C. The resulting mixture was stirred at rt for 1 h. The solvent was removed under reduced pressure, diluted with EtOAc and washed with water. The combined organic layer was dried over Na₂SO₄, and concentrated under reduced pressure to give 2-(2-bromo-4-phenoxy-phenyl)-ethanol which was used for the next step without further purification. ¹H NMR (400 MHz, CDCl₃) δ 7.37 (t, J=6.38 Hz, 2H), 7.20 (m, 2H), 7.13 (d, J=4.0 Hz, 1H), 7.02 (dd, J=8.4, 1.2 Hz, 2H), 6.90 (dd, J=8.0, 2.0 Hz, 1H), 3.88 (q, J=12.4, 6.8 Hz, 2H), 3.00 (t, J=6.6 Hz, 2H).

### Step 4. 2-Bromo-1-(2-methoxymethoxy-ethyl)-4-phenoxy-benzene

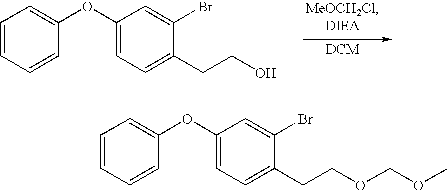

A solution of 2-(2-bromo-4-phenoxy-phenyl)-ethanol (0.32 g, 1.1 mmol) in DCM (15 mL) was cooled to 0° C. Diisopropylethylamine (0.17 g, 1.32 mmol) and chloromethylmethyl ether (0.11 g, 1.32 mmol) were added. The reaction mixture was stirred at rt overnight. The reaction mixture was extracted with DCM and washed with water, brine, dried over Na₂SO₄, and concentrated under reduced pressure to give crude product, which was purified by biotage (5% EtOAc in hexane) to afford 2-bromo-1-(2-methoxymethoxy-ethyl)-4-phenoxy-benzene (0.2 g, 54.5% yield) as a white solid. ¹H NMR (400 MHz, CDCl₃) δ 7.35 (t, J=8.0 Hz, 2H), 7.22 (d, J=8.4 Hz, 1H), 7.19 (d, J=2.4 Hz, 1H), 7.13 (t, J=7.4 Hz, 1H), 7.00 (dd, J=8.8, 1.6 Hz, 2H), 6.89 (dd, J=8.8, 2.8 Hz, 1H), 4.75 (s, 2H), 3.76 (t, J=7.0 Hz, 2H), 3.30 (s, 3H), 3.02 (t, J=7.0 Hz, 2H).

### Step 5. 2-[2-(2-Methoxymethoxy-ethyl)-5-phenoxy-phenyl]-4,4,5,5-tetramethyl-[1,3,2]dioxaborolane

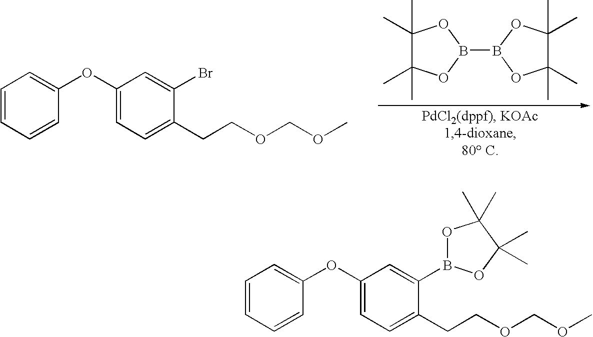

To a solution of 2-bromo-1-(2-methoxymethoxy-ethyl)-4-phenoxy-benzene (0.19 g, 0.56 mmol) in 1,4-dioxane (10 mL) was added bis(pinacolato)diboron (0.29 g, 1.13 mmol), potassium acetate (0.22 g, 2.24 mmol), and [1,1′-bis(diphenylphosphino)ferrocene]-palladium(II)chloride (0.02 g, 0.028 mmol). Nitrogen gas was passed through the mixture for 10 min and the suspension was heated at 80° C. overnight. The reaction mixture was extracted with EtOAc and washed with water, brine, dried over Na₂SO₄, and concentrated under reduced pressure to give crude product, which was purified by biotage (5-100% EtOAc in hexane) to afford 2-[2-(2-Methoxymethoxy-ethyl)-5-phenoxy-phenyl]-4,4,5,5-tetramethyl-[1,3,2]dioxaborolane (0.18 g, 83% yield) as a white semi solid. ¹H NMR (400 MHz, CDCl₃) δ 7.48 (d, J=2.8 Hz, 1H), 7.29 (t, J=8.0 Hz, 3H), 7.20 (d, J=8.0 Hz, 1H), 7.04 (m, 1H), 6.99 (dd, J=8.0, 2.8 Hz, 1H), 6.94 (d, J=8.8 Hz, 1H), 4.62 (s, 2H), 3.70 (t, J=7.2, Hz, 2H), 3.30 (s, 3H), 3.18 (t, J=7.2 Hz, 2H), 1.32 (s, 12H).

### Step 6. 7-Phenoxy-3,4-dihydro-benzo[c][1,2]oxaborinin-1-ol

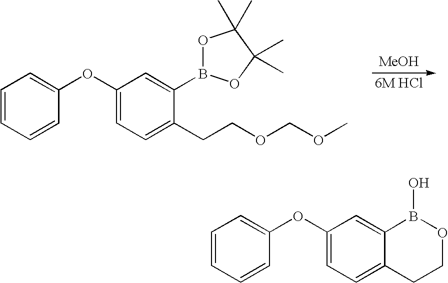

To a solution of 2-[2-(2-methoxymethoxy-ethyl)-5-phenoxy-phenyl]-4,4,5,5-tetramethyl-[1,3,2]dioxaborolane (0.18 g, 0.468 mmol) in methanol (5 mL) was added 6N HCl (5 mL). The resulting mixture was refluxed overnight. The reaction mixture was extracted with DCM and washed with water, brine, dried over Na₂SO₄, and concentrated under reduced pressure to give crude product, which was purified by biotage (50% EtOAc in hexane) to afford 7-phenoxy-3,4-dihydro-benzo[c][1,2]oxaborinin-1-ol (0.045 g, 40% yield) as a white semi solid. ¹H NMR (400 MHz, CDCl₃) δ 7.39 (d, J=2.8 Hz, 1H), 7.35 (t, J=7.4 Hz, 2H), 7.18 (d, J=8.0 Hz, 1H), 7.09 (m, 2H), 7.00 (d, J=8.0 Hz, 2H). MS (ESI) m/z=239 [M−H]⁻.

### E150 3-(1-Amino-2-hydroxyethyl)benzo[c][1,2]oxaborol-1(3H)-ol

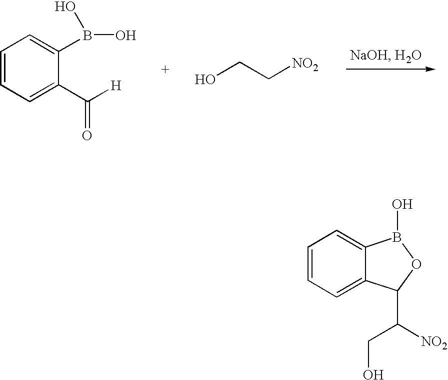

To a solution of NaOH (4.8 g, 119.88 mmol) in 96 ml of water was added 2-formyl benzene boronic acid (15 g, 99.9 mmol) by stirring at rt for 10 min. To the reaction mixture, 2-nitromethane (10.92 g, 119.88 mmol) was added dropwise. The solution was stirred for another 30 min. The reaction mixture was cooled to 5° C. and 3N HCl (10 mL) was added dropwise until pH of 2 was attained. Then extracted with EtOAc, washed with water, dried, concentrated. Chromatography (hexane/EtOAc 1:1) to get the target molecule 11 g. ¹H NMR (DMSO-d₆, 300 MHz) δ ppm 9.48 (s, 1H), 7.69 (dd, J=7.5 Hz, 0.8 Hz, 1H), 7.54 (m, 2H), 7.39 (d, J=6.9 Hz, 1 Hz, 1H), 5.58 (d, J=5.4 Hz, 1H), 5.49 (br, 1H), 5.02 (m, 1H), 4.18 (d, J=6 Hz, 1H), and 4.40 (dd, J=12, 3.6 Hz, 1H). MS (ESI) m/z 222.1 [M−H]⁻.

### E151 4-Phenyl-3,4-dihydro-benzo[c][1,2]oxaborinin-1-ol

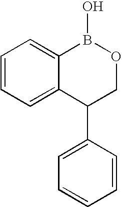

### Step 1. (2-Bromo-phenyl)-phenyl-acetaldehyde

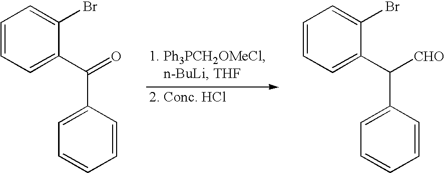

To a solution of (methoxymethyl)triphenylphosphonium chloride (6.6 g, 19.15 mmol) in THF (40 mL) at 0° C. was added n-BuLi (12 mL, 19.15 mmol) dropwise, and stirred at room temperature for 20 min. (2-Bromo-phenyl)-phenyl-methanone in 7 mL of THF was added dropwise to the reaction mixture and stirred at room temperature for 1 h. The reaction mixture was quenched with water, extracted with ether, washed with brine, dried over Na₂SO₄, and concentrated under reduced pressure to give crude product, which was purified by biotage (1:20 Ether:hexane) to give 1.5 g of colorless oil, which was dissolved in 4 mL of concentrated HCl. The reaction mixture was heated at 70° C. for 1 h, extracted with EtOAc, washed with water, brine, dried over Na₂SO₄, and concentrated under reduced pressure to give (2-bromo-phenyl)-phenyl-acetaldehyde (1.0 g, 94% yield) as a colorless oil. ¹H NMR (400 MHz, CDCl₃) δ 9.98 (s, 1H), 7.64 (dd, J=8.4, 1.6 Hz, 1H), 7.40-7.28 (m, 5H), 7.24-7.11 (m, 3H), 5.46 (s, 1H).

### Step 2. 2-(2-Bromo-phenyl)-2-phenyl-ethanol

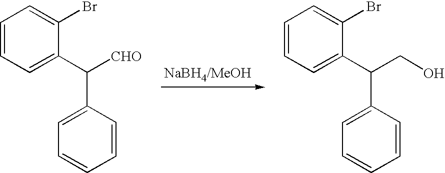

To a solution of (2-bromo-phenyl)-phenyl-acetaldehyde (1.0 g, 3.6 mmol) in methanol (20 mL) was added sodium borohydride (0.165 g, 4.4 mmol) at 0° C. The resulting mixture was stirred at rt for 1 h. The solvent was removed under reduced pressure, diluted with EtOAc and washed with water. The combined organic layer was dried over Na₂SO₄, and concentrated under reduced pressure to give 2-(2-bromo-phenyl)-2-phenyl-ethanol (1.0 g, quantitative) which was used for the next step without further purification. ¹H NMR (400 MHz, CDCl₃) δ 7.58 (dd, J=8.0, 1.2 Hz, 1H), 7.34-7.20 (m, 7H), 7.12 (m, 1H), 4.70 (t, J=7.0 Hz, 1H), 4.17 (d, J=6.8 Hz, 2H).

### Step 3. 1-Bromo-2-(2-methoxymethoxy-1-phenyl-ethyl)-benzene

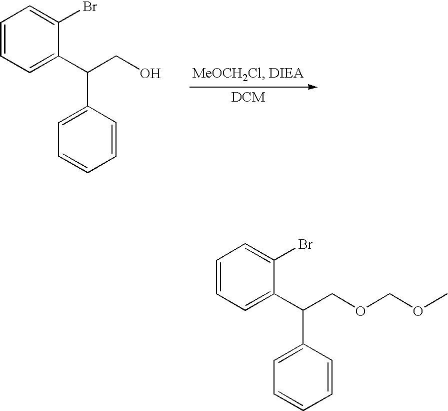

To a solution of 2-(2-bromo-phenyl)-2-phenyl-ethanol (1.0 g, 3.6 mmol) in DCM (20 mL) was cooled to 0° C. Diisopropylethylamine (0.56 g, 4.33 mmol) and chloromethylmethyl ether (0.35 g, 4.33 mmol) was added. The reaction mixture was stirred at rt overnight. The reaction mixture was extracted with DCM and washed with water, brine, dried over Na₂SO₄, and concentrated under reduced pressure to give crude product, which was purified by biotage (20% EtOAc in hexane) to afford 1-bromo-2-(2-methoxymethoxy-1-phenyl-ethyl)-benzene (0.54 g, 47% yield) as a colorless oil. ¹H NMR (400 MHz, CDCl₃) δ 7.55 (d, J=8.0 Hz, 1H), 7.31-7.19 (m, 7H), 7.10-7.06 (m, 1H), 4.82 (t, J=7.4 Hz, 1H), 4.62 (s, 2H), 4.11-4.01 (m, 2H), 3.25 (s, 3H).

### Step 4. 2-[2-(2-Methoxymethoxy-1-phenyl-ethyl)-phenyl]-4,4,5,5-tetramethyl-[1,3,2]dioxaborolane

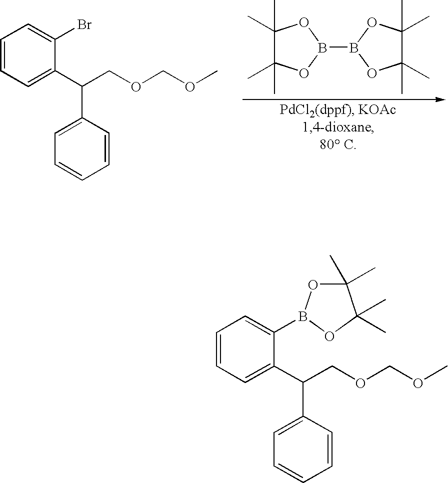

1-Bromo-2-(2-methoxymethoxy-1-phenyl-ethyl)-benzene (0.54 g, 1.68 mmol) in 1,4-dioxane (15 mL) was degassed for 30 min under nitrogen gas. Bis(pinacolato)diboron (0.85 g, 3.36 mmol), potassium acetate (0.66 g, 6.72 mmol), and [1,1′-bis(diphenylphosphino)ferrocene]palladium(II)chloride (0.062 g, 0.084 mmol) were added. The reaction mixture was heated at 80° C. overnight. The reaction mixture was extracted with EtOAc and washed with water, brine, dried over Na₂SO₄, and concentrated under reduced pressure to give crude product, which was purified by biotage (5-20% EtOAc in hexane) to afford 2-[2-(2-Methoxymethoxy-1-phenyl-ethyl)-phenyl]-4,4,5,5-tetramethyl-[1,3,2]dioxaborolane (0.32 g, 52% yield) as a white solid. ¹H NMR (400 MHz, CDCl₃) δ 7.77 (d, J=8.0 Hz, 1H), 7.65 (d, J=7.6 Hz, 1H), 7.41-7.13 (m, 7H), 5.28 (t, J=7.2 Hz, 1H), 4.70 (s, 2H), 3.86 (d, J=6.8 Hz, 2H), 3.34 (s, 3H), 1.31 (s, 12H).

### Step 5. 4-Phenyl-3,4-dihydro-benzo[c][1,2]oxaborinin-1-ol

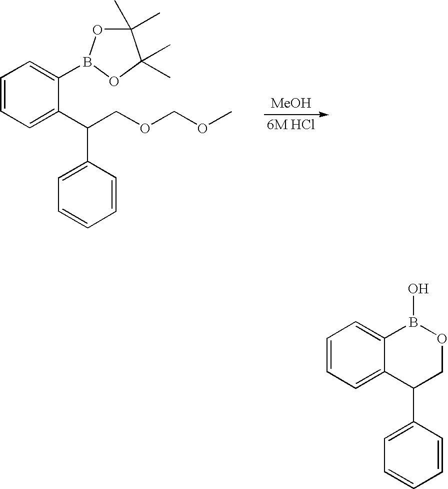

To a solution of 2-[2-(2-methoxymethoxy-1-phenyl-ethyl)-phenyl]-4,4,5,5-tetramethyl-[1,3,2]dioxaborolane (0.32 g, 0.87 mmol) in methanol (10 mL) was added 6N HCl (5 mL). The resulting mixture was refluxed overnight. The reaction mixture was extracted with DCM and washed with water, brine, dried over Na₂SO₄, and concentrated under reduced pressure to give crude product, which was purified by preparative HPLC (to afford 4-phenyl-3,4-dihydro-benzo[c][1,2]oxaborinin-1-ol (0.025 g, 13% yield) as an off white solid. mp 83-85° C. ¹H NMR (400 MHz, CDCl₃) δ 7.78 (d, J=7.6 Hz, 2H), 7.62 (dd, J=7.6, 1.2 Hz, 2H), 7.42-7.39 (m, 2H), 7.35-7.31 (m, 3H), 4.22 (s, 1H), 4.20-4.10 (m, 3H). MS (ESI) m/z=223 [M−H]⁻.

### E152 2-Nitromethyl-7,8-dihydro-2H-1,6,9-trioxa-9a-bora-benzo[cd]azulene

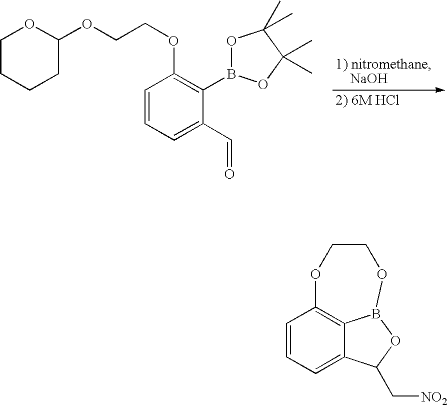

To a solution of NaOH (0.027 g, 0.683 mmol) in water (1.35 mL) was added nitromethane (0.104 g, 1.71 mmol) at 5-10° C. After stirring at for 5 min at 5-10° C., ACTBr (0.0124 g, 0.034 mmol) was added to the reaction mixture and followed by the addition of 3-[2-(tetrahydro-pyran-2-yloxy)-ethoxy]-2-(4,4,5,5-tetramethyl-[1,3,2]dioxaborolan-2-yl)-benzaldehyde (0.257 g, 0.683 mmol) at 5-10° C. The reaction mixture was stirred at 15° C. for 5 h. The reaction mixture was acidified to pH 1 using diluted hydrochloric acid and stirred at room temperature overnight. The reaction mixture was diluted with EtOAc, washed with brine, dried and concentrated to dryness. The residue was purified by recrystalized from EtOAc/hexanes to give 0.062 g of product as white solid. Mp 115-118° C. ¹HNMR (400 MHz, DMSO-d₆) δ 7.47 (dd, J=8.0 Hz, 8.4 Hz, 1H), 7.13 (d, J=8.0 Hz, 1H), 6.89 (d, J=8.4 Hz, 1H), 5.89 (br. m, 1H), 5.33 (br. m, 1H), 4.67-4.61 (m, 2H), 4.34-4.30 (m, 2H), 4.18 (br. m, 1H). Calc. for C₁₀H₁₀BNO₅: C 51.11% yield, H 4.29% yield, N 5.96% yield; Found: C 51.00% yield, H 4.36% yield, N 5.99% yield.

### E153 6-Benzyl-3H-benzo[c]oxaborol-1-ol

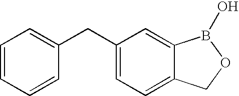

### Step 1. 4-Benzyl-2-methoxy-benzaldehyde

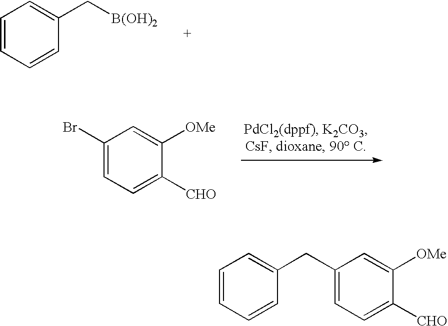

A mixture of benzylboronic acid (2.15 g, 10 mmol), 4-bromo-2-methoxy-benzaldehyde (2.44 g, 18 mmol), Pd(dppf)Cl₂ (1.46 g , 2 mmol), CsF (3.02 g, 20 mmol) and K₂CO₃ (4.14 g, 30 mmol) in dioxane (30 mL) was degassed for 10 min and heated at 80° C. for 16 h, cooled to RT, diluted with EtOAc, filtered through a pad of Celite and concentrated. The residue was purified by chromatography to give 4-benzyl-2-methoxy-benzaldehyde (2.24 g, 100% yield). ¹HNMR (400 MHz, CDCl₃) δ 10.40 (s, 1H), 7.77 (d, J=8.2 Hz, 1H), 7.30-7.10 (m, 5H), 6.85 (d, J=8.2 Hz, 1H), 6.77 (s, 1H), 4.03 (s, 2H), 3.85 (s, 3H).

### Step 2. 4-Benzyl-2-hydroxy-benzaldehyde

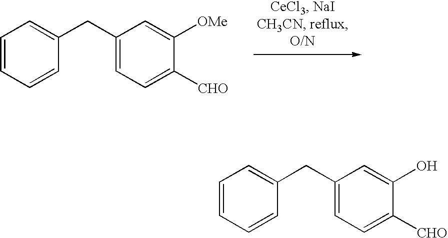

A mixture of 4-benzyl-2-methoxy-benzaldehyde (1.14 g, 5 mmol), CeCl₃ (1.85 g, 7.5 mmol) and NaI (1.13 g, 7.5 mmol) in CH₃CN (20 mL) was refluxed for 18 h, diluted with EtOAc, washed with aqueous Na₂S₂O₄, dried and concentrated to give 4-benzyl-2-hydroxy-benzaldehyde (1.10 g, 100% yield). ¹HNMR (400 MHz, CDCl₃) δ 11.00 (s, 1H), 9.80 (s, 1H), 7.47 (d, J=8.1 Hz, 1H), 7.30-7.10 (m, 5H), 6.82 (m, 2H), 3.98 (s, 2H).

### Step 3. Trifluoro-methanesulfonic acid 5-benzyl-2-formyl-phenyl ester

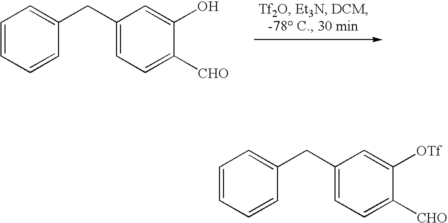

To a cooled (−78° C.) solution of 4-benzyl-2-hydroxy-benzaldehyde (0.44 g, 2.08 mmol) in dichloromethane (10 mL) was added Et₃N (0.68 mL, 6.24 mmol) and then Tf₂O (0.40 mL, 3.12 mmol). The mixture was stirred at −78° C. for 30 min, quenched with H₂O (2 mL), diluted with dichloromethane (50 mL), washed with 1 N HCl (20 mL), dried and concentrated to give trifluoro-methanesulfonic acid 5-benzyl-2-formyl-phenyl ester (0.68 g, 100% yield). ¹HNMR (400 MHz, CDCl₃) δ 10.20 (s, 1H), 7.90 (d, J=7.7 Hz, 1H), 7.40-7.00 (m, 7H), 4.00 (s, 2H).

### Step 4. 4-Benzyl-2-(4,4,5,5-tetramethyl-[1,3,2]dioxaborolan-2-yl)benzaldehyde

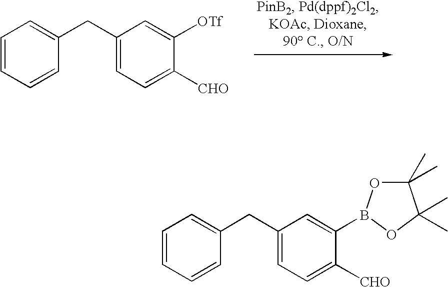

A mixture of trifluoro-methanesulfonic acid 5-benzyl-2-formyl-phenyl ester (0.68 g, 2.08 mmol), bis(pinocolato)diborane (0.80 g, 3.12 mmol), Pd(dppf)Cl₂ (0.31 g, 0.42 mmol) and KOAc (0.61 g, 6.24 mmol) in dioxane (15 mL) was heated at 80° C. for 16 h, cooled to RT, diluted with EtOAc, filtered through a pad of Celite and concentrated. The residue was purified by chromatography to give 4-benzyl-2-(4,4,5,5-tetramethyl-[1,3,2]dioxaborolan-2-yl)benzaldehyde (0.61 g, 90% yield). ¹HNMR (400 MHz, CDCl₃) δ 10.50 (s, 1H), 7.90 (d, J=8.2 Hz, 1H), 7.70 (s, 1H), 7.40-7.10 (m, 6H), 4.02 (s, 2H), 1.40 (s, 12H).

### Step 5. 6-Benzyl-3H-benzo[c]oxaborol-1-ol

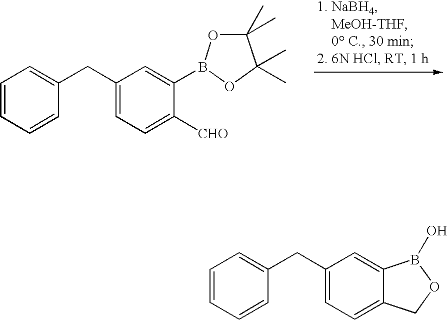

To a cooled (0° C.) solution of 4-benzyl-2-(4,4,5,5-tetramethyl-[1,3,2]dioxaborolan-2-yl)benzaldehyde (0.61 g, 1.89 mmol) in MeOH (10 mL) and THF (10 mL) was added NaBH₄ (0.16 g, 4.17 mmol) in portions. After the addition was over, the mixture was stirred at 0° C. for 30 min, quenched with 6 N HCl (0.5 mL) and diluted with H₂O (20 mL). The mixture was stirred at RT for 1 h. The solid formed was collected, washed with H₂O (10 mL) and dried under high vacuum to give 6-benzyl-3H-benzo[c]oxaborol-1-ol (290 mg, 68% yield). Mp 173-175° C. ¹HNMR (DMSO-d₆, 400 MHz) δ 9.10 (s, 1H), 7.58 (s, 1H), 7.40-7.10 (m, 7H), 4.92 (s, 2H), 3.97 (s, 2H). MS (ESI) m/z=223 [M−H]⁻.

### Example 2

### Testing of Compounds for the Biochemical and Micriobial Inhibition of β-Lactamases

All β-lactamases were tested as essentially described by Payne et al., J. Antimicrob. Chemother., 1991; 28: 775-776) with a few modifications. The buffer was 50 mM potassium phosphate pH 7 with 0.2% Triton x-100, and the concentration of nitrocefin was 500 μM for class A β-lactamases and 200 μM for class C β-lactamases. Kinetic data is collected by measuring the rate of change in A₄₈₆ over 30 minutes. The fraction of enzyme inhibited is determined by dividing the reaction rates in the presence of inhibitor by the reaction rate determined in the absence of inhibitor. Dose-response curves are then generated by plotting log [inhibitor] vs. fraction inhibited. IC₅₀ values were determined from the dose-response curves by determining the inhibitor concentration required to reduce the maximum inhibitory activity of the compound by 50%. The Kᵢ values were calculated from the IC50 using the Kₘ for nitrocefin for each enzyme and the following equation

$${K}_{i} = \frac{{IC}_{50}}{1 + \frac{\vert S \vert}{{K}_{m}}} .$$

AmpC P99 was purchased from Sigma-Aldrich #P4399, TEM-1 was purchased from Invitrogen #PV3575, and CTX-M-9 was obtained from Professor Brian Shoichet of the University of California-San Francisco (Yu Chen, Brian Shoichet, and Richard Bonnet, J. Am. Chem. Soc., 2005, 127 (15): pp 5423-5434).

CMY-2 was synthesized by GenScript and subcloned into pET24b at the NdeI/SalI sites. The DNA sequence of the insert is SEQ ID NO: 25 and is as follows,

CATATGATGAAAAAATCGTTATGCTGCGCTCTGCTGCTGACAGCCTCTTT
CTCCACATTT
GCTGCCGCAAAAACAGAACAACAGATTGCCGATATCGTTAATCGCACCAT
CACCCCGTTG
ATGCAGGAGCAGGCTATTCCGGGTATGGCCGTTGCCGTTATCTACCAGGG
AAAACCCTAT
TATTTCACCTGGGGTAAAGCCGATATCGCCAATAACCACCCAGTCACGCA
GCAAACGCTG
TTTGAGCTAGGATCGGTTAGTAAGACGTTTAACGGCGTGTTGGGCGGCGA
TGCTATCGCC
CGCGGCGAAATTAAGCTCAGCGATCCGGTCACGAAATACTGGCCAGAACT
GACAGGCAAA
CAGTGGCAGGGTATCCGCCTGCTGCACTTAGCCACCTATACGGCAGGCGG
CCTACCGCTG
CAGATCCCCGATGACGTTAGGGATAAAGCCGCATTACTGCATTTTTATCA
AAACTGGCAG
CCGCAATGGACTCCGGGCGCTAAGCGACTTTACGCTAACTCCAGCATTGG
TCTGTTTGGC
GCGCTGGCGGTGAAACCCTCAGGAATGAGTTACGAAGAGGCAATGACCAG
ACGCGTCCTG
CAACCATTAAAACTGGCGCATACCTGGATTACGGTTCCGCAGAACGAACA
AAAAGATTAT
GCCTGGGGCTATCGCGAAGGGAAGCCCGTACACGTTTCTCCGGGACAACT
TGACGCCGAA
GCCTATGGCGTGAAATCCAGCGTTATTGATATGGCCCGCTGGGTTCAGGC
CAACATGGAT
GCCAGCCACGTTCAGGAGAAAACGCTCCAGCAGGGCATTGCGCTTGCGCA
GTCTCGCTAC
TGGCGTATTGGCGATATGTACCAGGGATTAGGCTGGGAGATGCTGAACTG
GCCGCTGAAA
GCTGATTCGATCATCAACGGCAGCGACAGCAAAGTGGCATTGGCAGCGCT
TCCCGCCGTT
GAGGTAAACCCGCCCGCCCCCGCAGTGAAAGCCTCATGGGTGCATAAAAC
GGGCTCCACT
GGTGGATTTGGCAGCTACGTAGCCTTCGTTCCAGAAAAAAACCTTGGCAT
CGTGATGCTG
GCAAACAAAAGCTATCCTAACCCTGTCCGTGTCGAGGCGGCCTGGCGCAT
TCTTGAAAAG
CTGCAATAAGTCGAC

KPC-2 was synthesized by GenScript and subcloned into pET24b at the NdeI/SalI sites. The DNA sequence of the insert is SEQ ID NO: 26 and is as follows,

CATATGTCACTGTATCGCCGTCTAGTTCTGCTGTCTTGTCTCTCATGGCC
GCTGGCTGGC
TTTTCTGCCACCGCGCTGACCAACCTCGTCGCGGAACCATTCGCTAAACT
CGAACAGGAC
TTTGGCGGCTCCATCGGTGTGTACGCGATGGATACCGGCTCAGGCGCAAC
TGTAAGTTAC
CGCGCTGAGGAGCGCTTCCCACTGTGCAGCTCATTCAAGGGCTTTCTTGC
TGCCGCTGTG
CTGGCTCGCAGCCAGCAGCAGGCCGGCTTGCTGGACACACCCATCCGTTA
CGGCAAAAAT
GCGCTGGTTCCGTGGTCACCCATCTCGGAAAAATATCTGACAACAGGCAT
GACGGTGGCG
GAGCTGTCCGCGGCCGCCGTGCAATACAGTGATAACGCCGCCGCCAATTT
GTTGCTGAAG
GAGTTGGGCGGCCCGGCCGGGCTGACGGCCTTCATGCGCTCTATCGGCGA
TACCACGTTC
CGTCTGGACCGCTGGGAGCTGGAGCTGAACTCCGCCATCCCAGGCGATGC
GCGCGATACC
TCATCGCCGCGCGCCGTGACGGAAAGCTTACAAAAACTGACACTGGGCTC
TGCACTGGCT
GCGCCGCAGCGGCAGCAGTTTGTTGATTGGCTAAAGGGAAACACGACCGG
CAACCACCGC
ATCCGCGCGGCGGTGCCGGCAGACTGGGCAGTCGGAGACAAAACCGGAAC
CTGCGGAGTG
TATGGCACGGCAAATGACTATGCCGTCGTCTGGCCCACTGGGCGCGCACC
TATTGTGTTG
GCCGTCTACACCCGGGCGCCTAACAAGGATGACAAGCACAGCGAGGCCGT
CATCGCCGCT
GCGGCTAGACTCGCGCTCGAGGGATTGGGCGTCAACGGGCAGTAAGTCGA
C

TEM-64 was synthesized by GenScript and subcloned into pET24b at the Nde I/Xho I sites. The DNA sequence of the insert is SEQ ID NO: 27 and is as follows,

CATATGAGTATTCAACATTTCCGTGTCGCCCTTATTCCGTTTTTTGCGGC
ATTTTGCCTTCCTGTTTTTGCTCACC
CAGAAACGCTGGTGAAAGTAAAAGATGCTGAAGATCAGTTGGGTGCACGA
GTGGGTTACATCGAACTGGATCTCAA
CAGCGGTAAGATCCTTGAGAGTTTTCGCCCGGAAGAACGTTTTCCAATGA
TGAGCACTTTTAAAGTTCTGCTGTGT
GGCGCGGTATTATCCCGTGTTGACGCCGGGCAAGAGCAACTCGGTCGCCG
CATTCACTATTCTCAGAATGACTTGG
TTAAGTACTCACCAGTCACAGAAAAGCATCTTACGGATGGCATGACAGTA
CGCGAATTATGCAGTGCTGCCATTAC
CATGAGTGATAACACTGCGGCCAACTTACTTCTGACAACGATCGGCGGCC
CGAAGGAGCTGACCGCTTTTTTGCAC
AACATGGGGGATCATGTAACTCGCCTTGATAGCTGGGAACCGGAGCTGAA
TGAAGCCATTCCAAACGACGAGCGTG
ACACCACGACCCCTGCAGCAATGGCAACAACGTTGCGCAAACTGTTAACT
GGCGAACTGCTTACTCTGGCTTCCCG
GCAACAATTAATTGACTGGATGGAGGCGGATAAAGTTGCAGGCCCACTTC
TGCGCTCGGCCCTTCCGGCTGGCTGG
TTTATTGCTGATAAATCTGGCGCCGGTGAGCGTGGGTCTCGCGGTATCAT
TGCAGCACTGGGGCCAGATGGTAAGC
CGTCCCGTATCGTAGTTATCTACACGACGGGGAGTCAGGCAACTATGGAT
GAACGAAATCGCCAGATCGCTGAGAT
TGGTGCCTCACTGATTAAGCATTGGCTCGAG

SHV-18 was synthesized by GenScript with the CMY-2 leader sequence and subcloned into pET24b at the Nde I/Xho I sites. The DNA sequence of the insert is SEO ID NO: 28 and is as follows,

CATATGATGAAAAAATCGTTATGCTGCGCTCTGCTGCTGACAGCCTCTTT
CTCCACATTTGCTGCCAGCCCGCAGCCGCTTGAGCAAATTAAACTAAGCG
AAAGCCAGCTGTCGGGCAGCGTAGGCATGATAGAAATGGATCTGGCCAGC
GGCCGCACGCTGACCGCCTGGCGCGCCGATGAACGCTTTCCCATGATGAG
CACCTTTAAAGTAGTGCTCTGCGGCGCAGTGCTGGCGCGGGTGGATGCCG
GTGACGAACAGCTGGAGCGAAAGATCCACTATCGCCAGCAGGATCTGGTG
GACTACTCGCCGGTCAGCGAAAAACACCTTGCCGACGGCATGACGGTCGG
CGAACTCTGTGCCGCCGCCATTACCATGAGCGATAACAGCGCCGCCAATC
TGCTGCTGGCCACCGTCGGCGGCCCCGCAGGATTGACTGCCTTTTTGCGC
CAGATCGGCGACAACGTCACCCGCCTTGACCGCTGGGAAACGGAACTGAA
TGAGGCGCTTCCCGGCGACGCCCGCGACACCACTACCCCGGCCAGCATGG
CCGCGACCCTGCGCAAGCTGCTGACCAGCCAGCGTCTGAGCGCCCGTTCG
CAACGGCAGCTGCTGCAGTGGATGGTGGACGATCGGGTCGCCGGACCGTT
GATCCGCTCCGTGCTGCCGGCGGGCTGGTTTATCGCCGATAAGACCGGAG
CTGCCAAACGGGGTGCGCGCGGGATTGTCGCCCTGCTTGGCCCGAATAAC
AAAGCAGAGCGGATTGTGGTGATTTATCTGCGGGATACGCCGGCGAGCAT
GGCCGAGCGAAATCAGCAAATCGCCGGGATCGGCGCGGCGCTGATCGAGC
ACTGGCAACGCTAACTCGAG

KPC-2, TEM-64, CMY-2, SHV-18 were over-expressed as essentially described for CTX-M-9 (Structure, Function, and Inhibition along the Reaction Coordinate of CTX-M β-Lactamases, Yu Chen, Brian Shoichet, and Richard Bonnet, J. Am. Chem. Soc., 2005, 127 (15), pp 5423-5434). Since β-lactamases are exported to the periplasm we obtained these enzymes by treating the cells with an osmotic shock. Cells were harvested by centrifugation at 4000×g for 20 minutes, the supernatant was discarded and the pellet was resuspended in 30 mM Tris-HCl, 20% sucrose, pH 8.0 (80 ml for each gram of cells wet weight) . Then EDTA was added to 1 mM and the cells were incubated for 5-10 minutes at room temperature with shaking The cells were then centrifuged at 8000×g for 20 minutes at 4° C., the supernatant was removed, and the pellet resuspended in ice-cold 5 mM MgSO₄ (80 ml for each gram of cells wet weight). The cells were incubated on ice for 10 minutes and then centrifuged at 8000×g for 20 minutes at 4° C. The supernatant was removed and dialyzed overnight at 4 C against 10 mM potassium phosphate pH 6.8, 50% glycerol. These partially purified enzyme preparations were used in for IC₅₀ determination.

The bacterial activity of our BLIs were screened by measuring the MIC of a β-lactam antibiotic in the presence of 4 μg/mL BLI using the Clinical and Laboratory

Standards Institute's microbroth dilution method in cation-adjusted Mueller-Hinton Broth (Methods for dilution Antimicrobial susceptibility tests for bacteria that grow aerobically M7-A7).

To test the synergistic activity, compounds were tested in a modified M7-A7 microbroth method, called a 2-D checkerboard assay. In a 96 well plate, lanes 1-11 contain 2-fold serial dilutions of the test compound usually starting at a concentration 64 μg/mL, while lanes A-G contain 2-fold serial dilutions of β-lactam antibiotic usually starting at a concentration 16 μg/mL. Lane 12 contains no test compound and lane H contains no β-lactam, therefore the dynamic range of the synergistic activity of the test compound can be tested in the presence of the β-lactam.

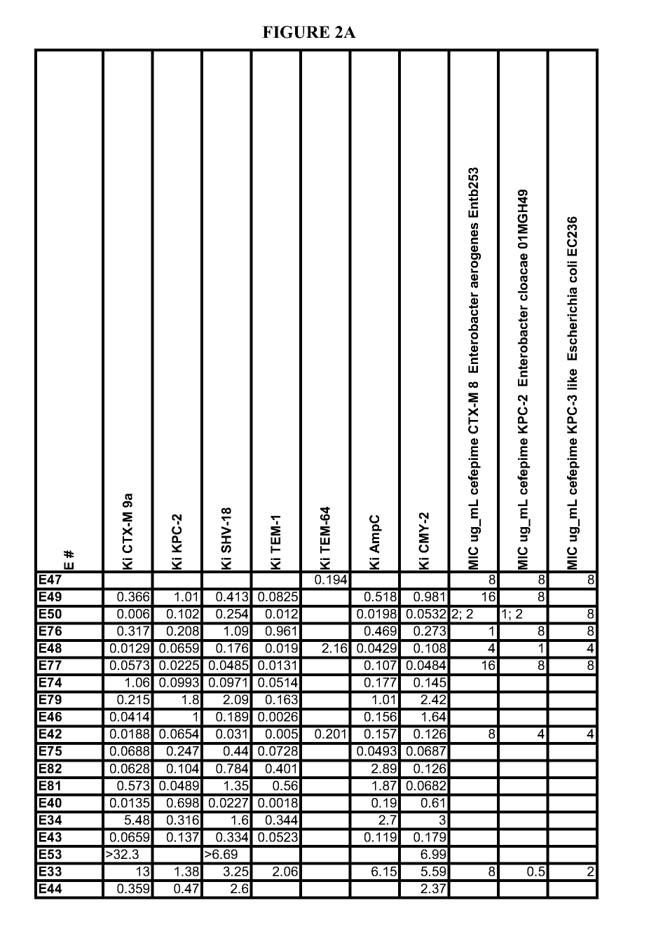

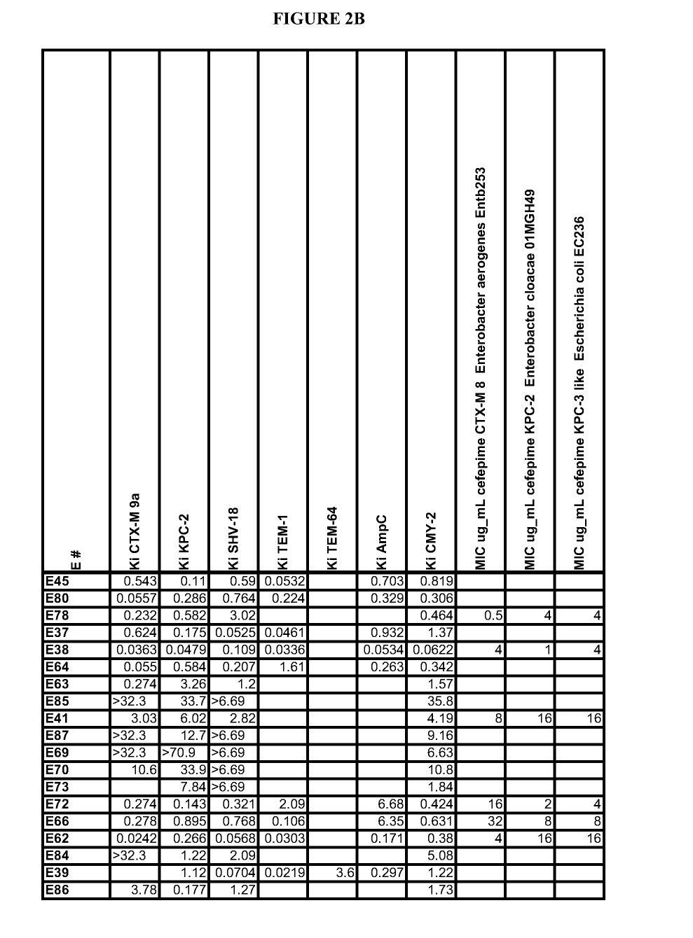

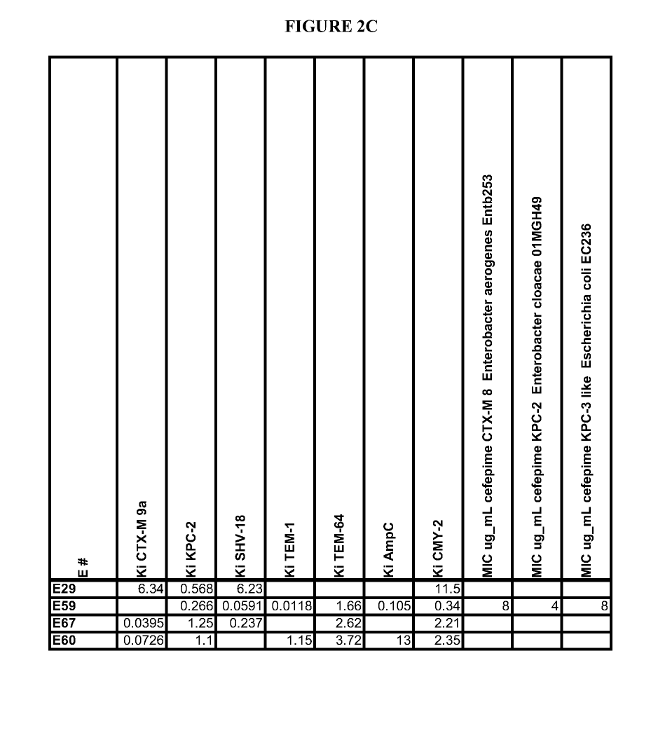

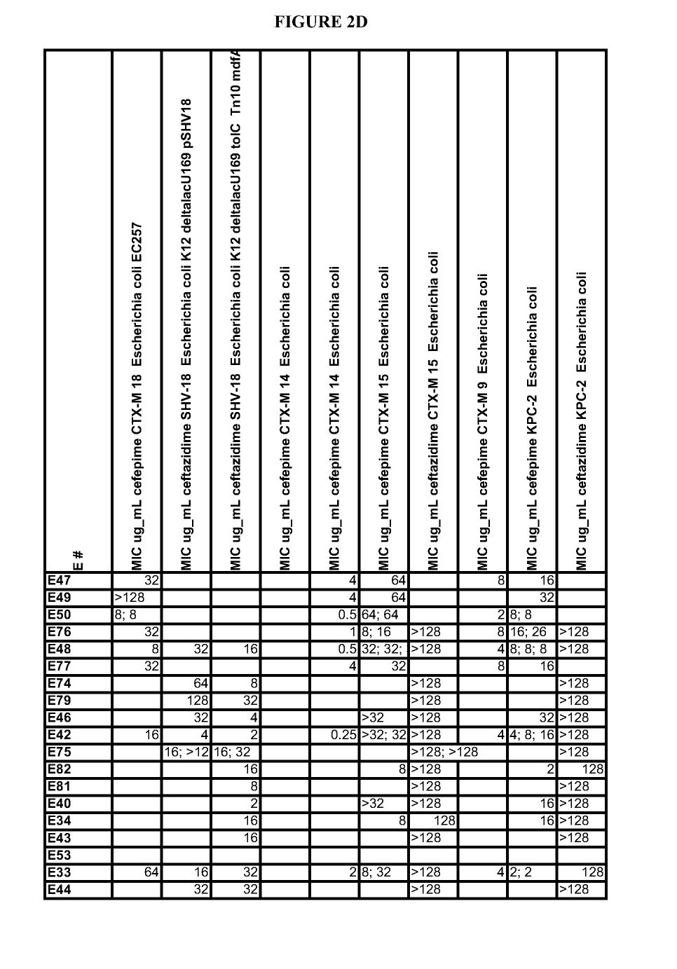

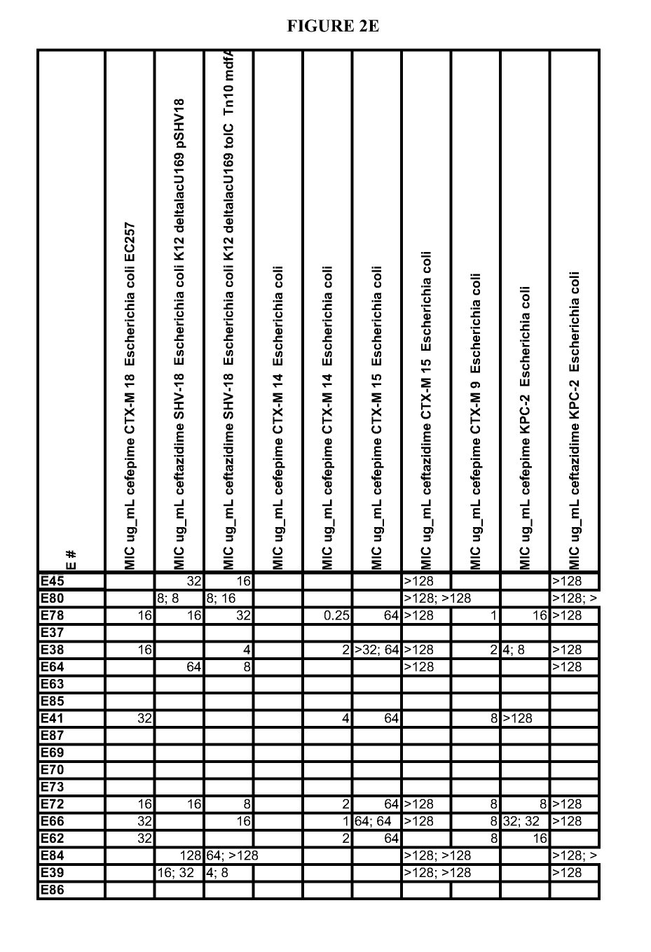

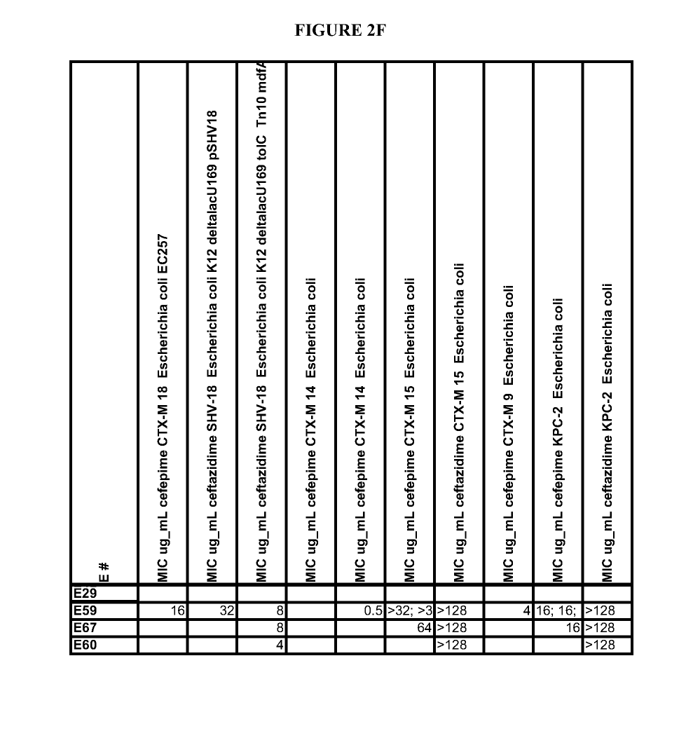

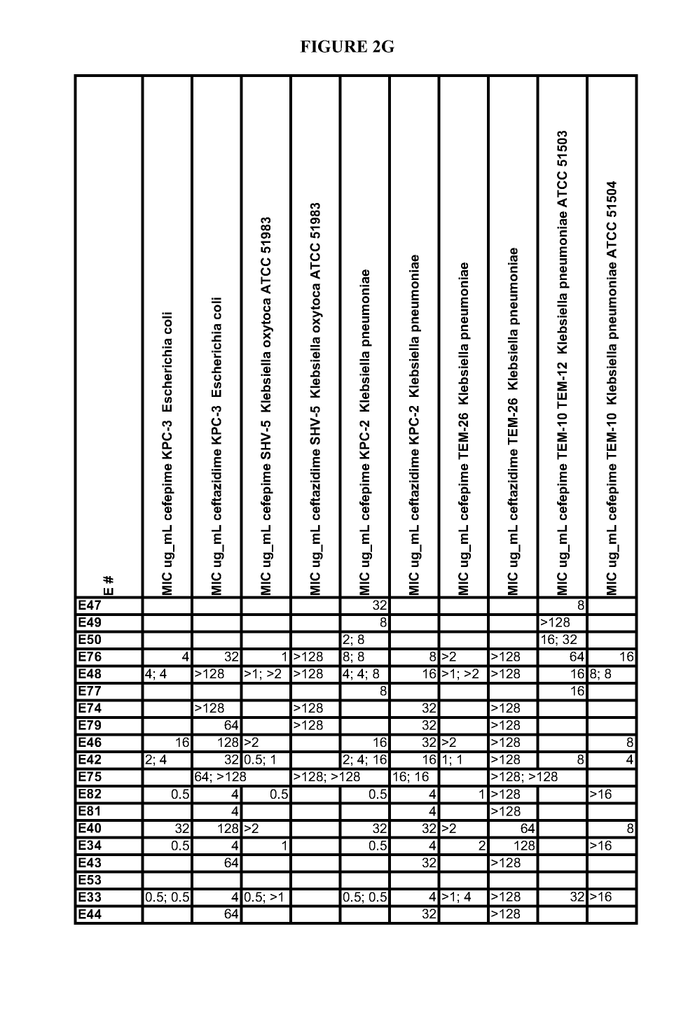

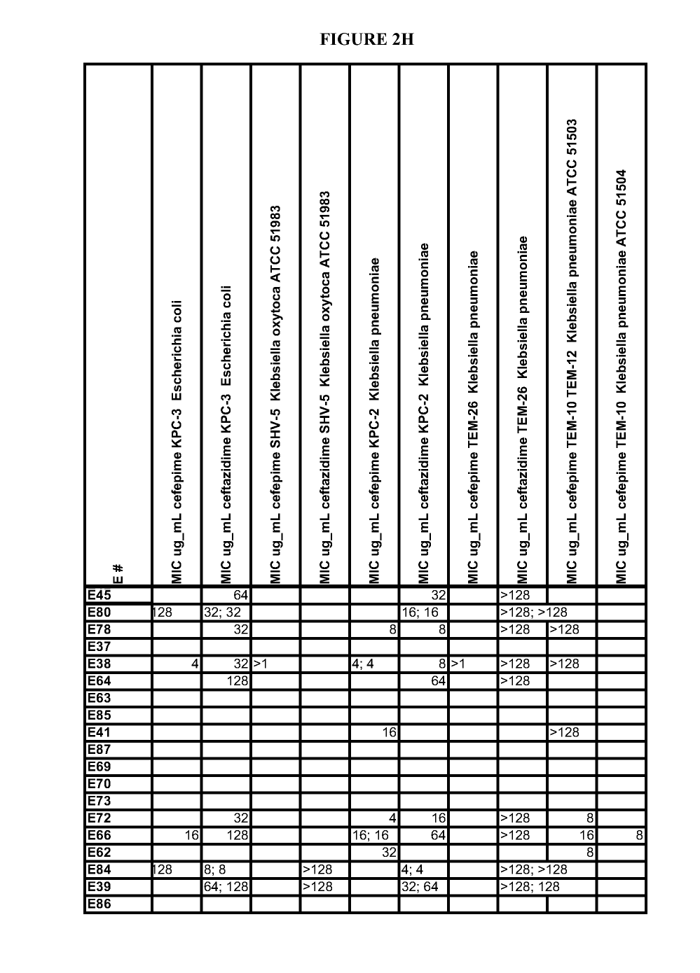

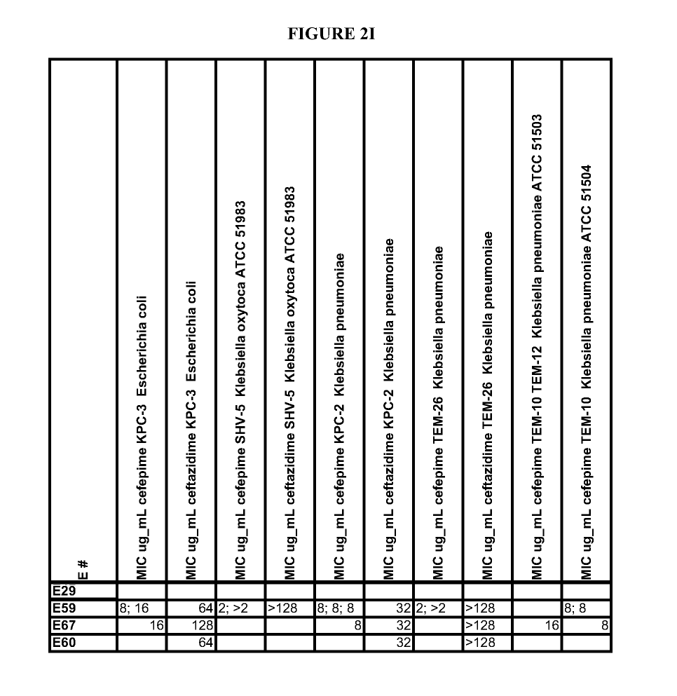

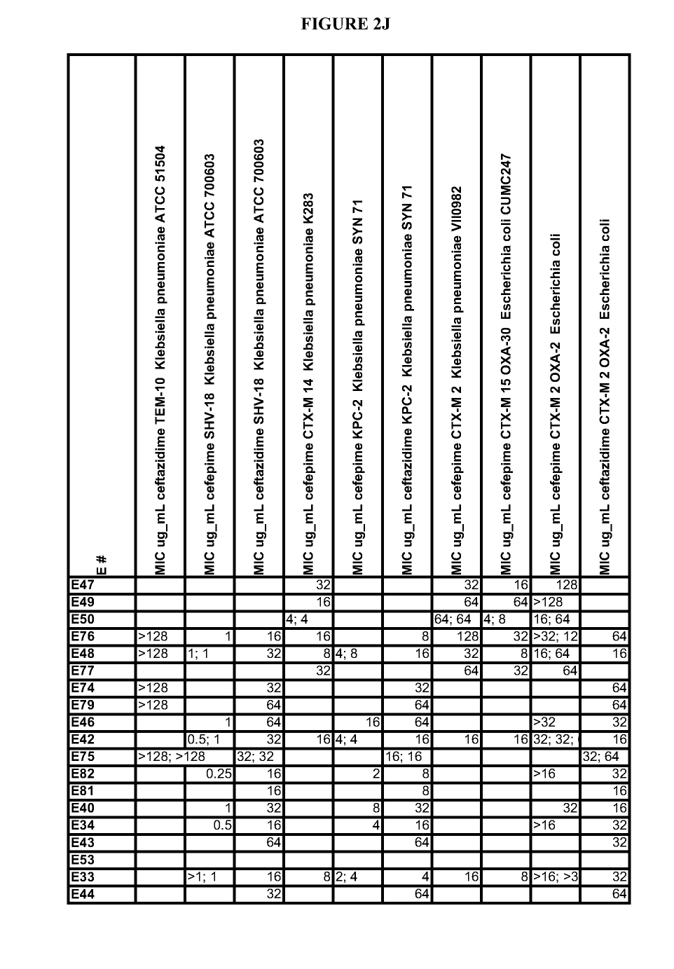

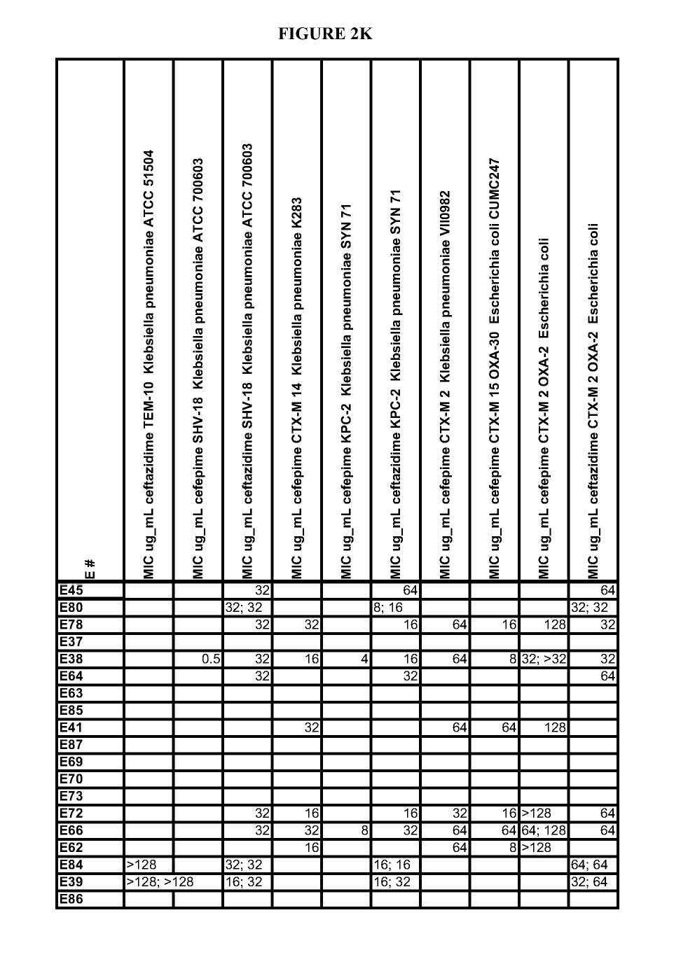

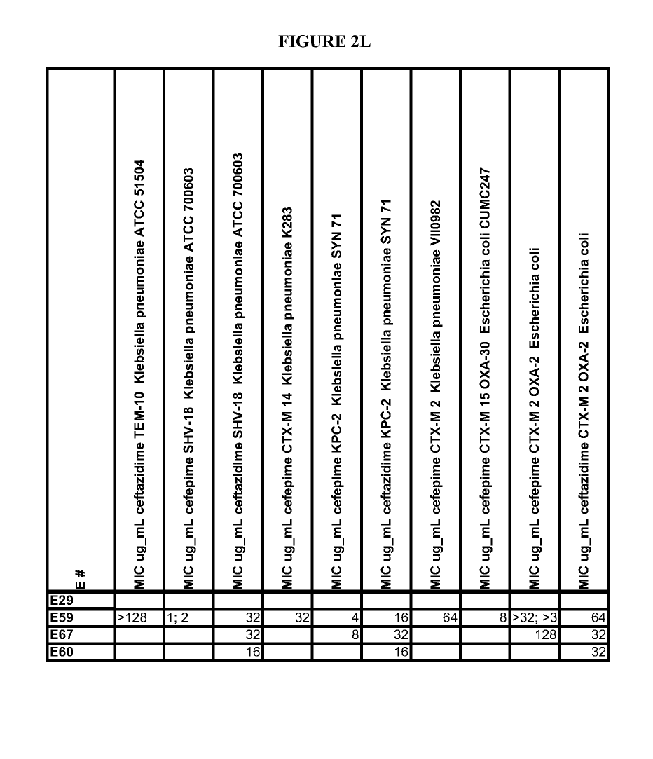

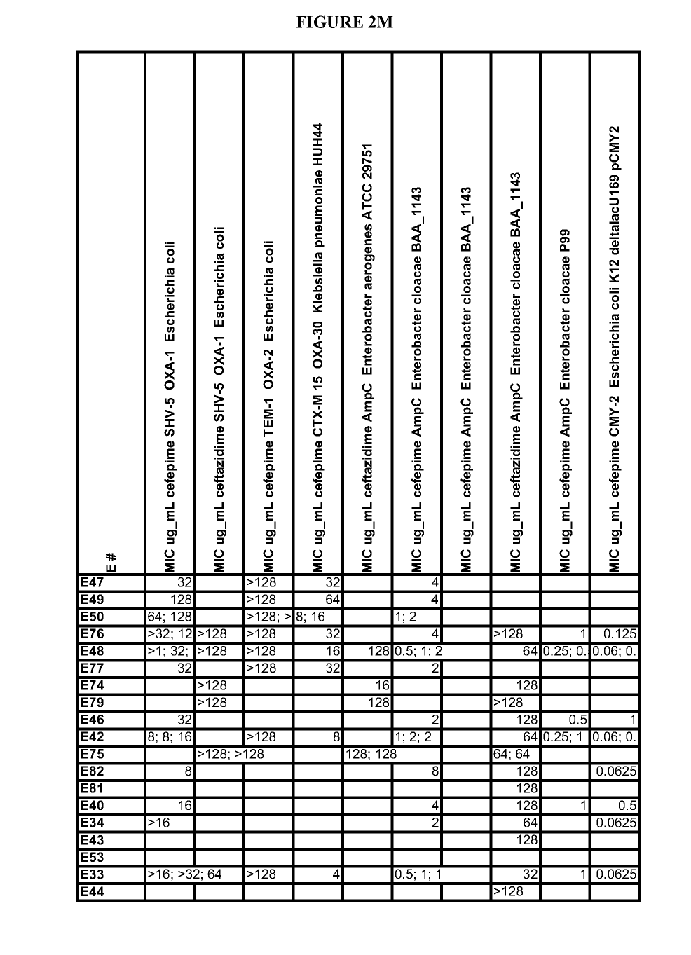

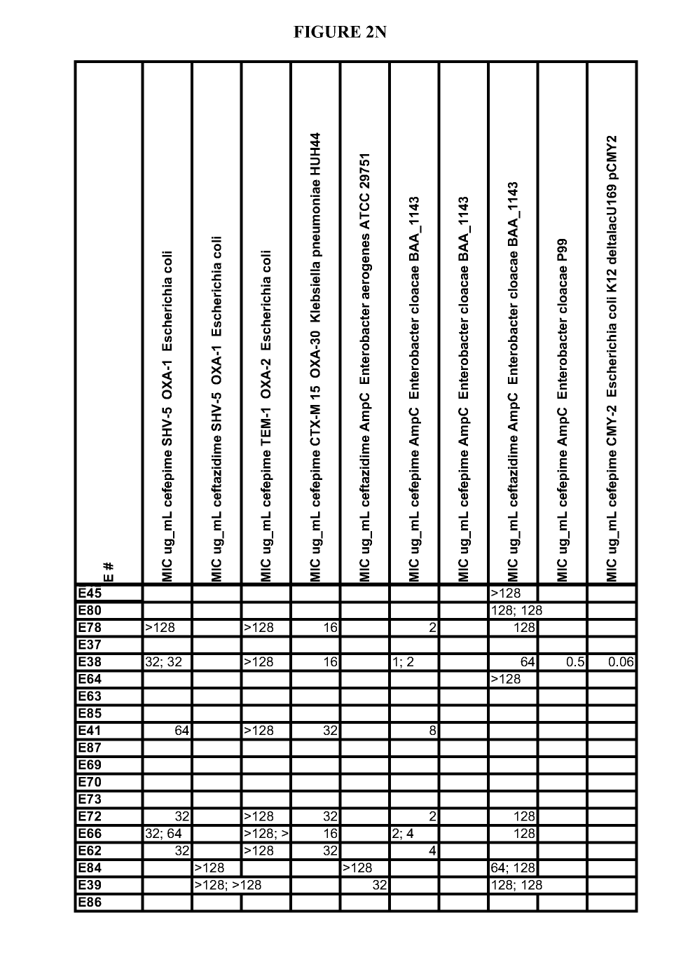

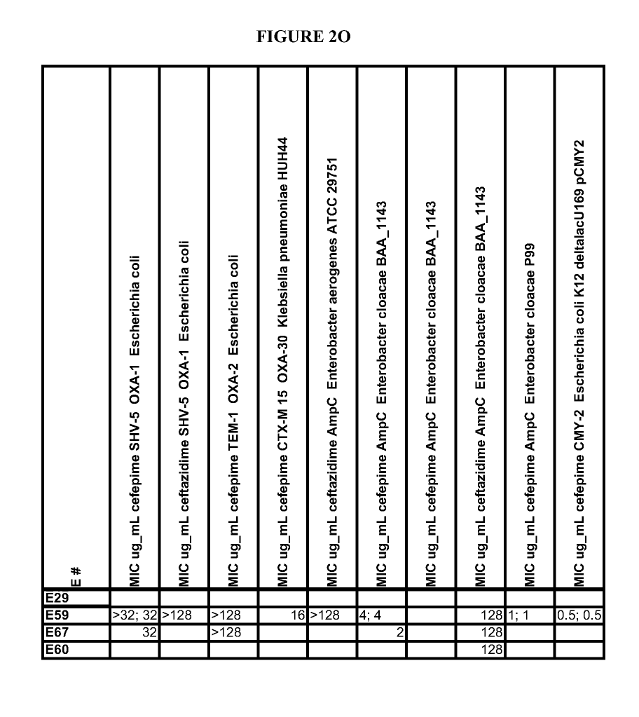

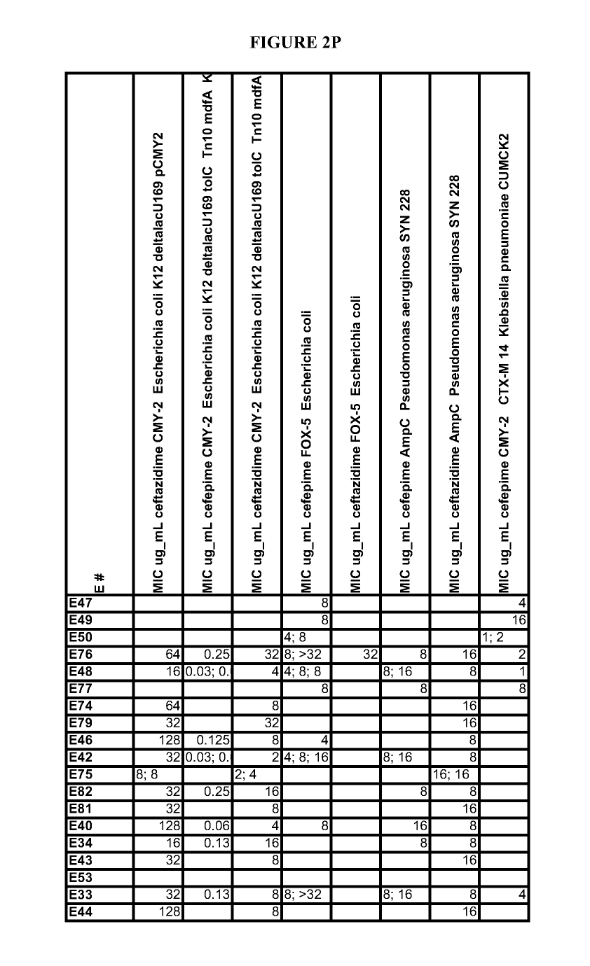

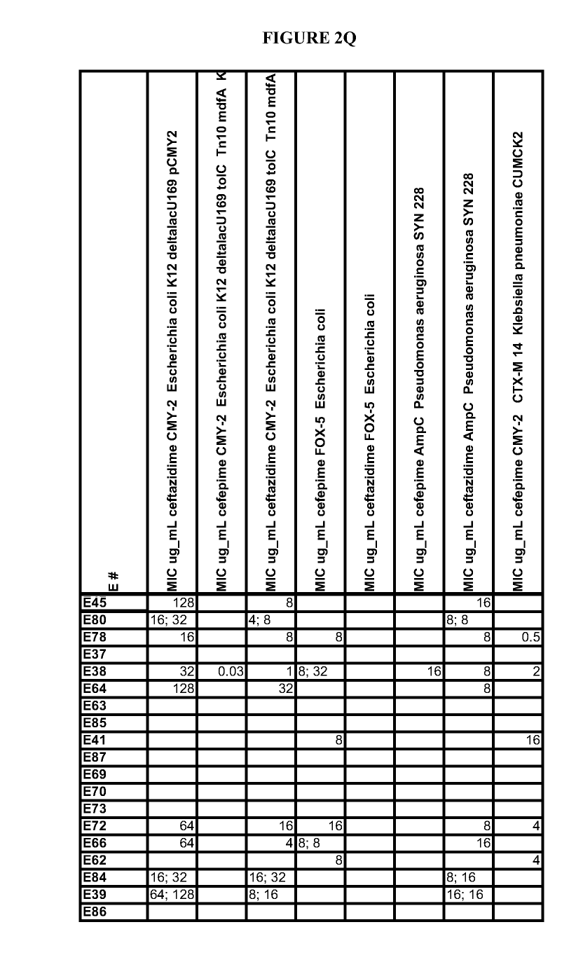

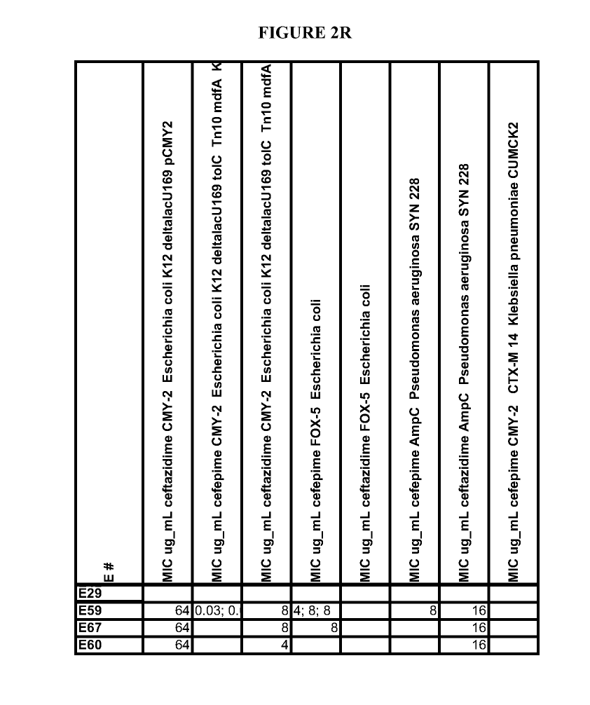

In vitro testing results for exemplary compounds of the invention are provided in FIG. 2.

### Example 3

Assay for Determining that a Compound Inhibits the Editing Domain of tRNA Synthetase in a Bacteria

This example sets forth a representative assay for determining whether a particular compound inhibits the editing domain of an ARS in a bacterium.

The [³H]-isoleucine mischarged tRNAleu was synthesized by incubating 1 μM of Saccharomyces cerevisiae editing defective Cdc60p (C326F) in 500 μL of 50 mM Tris-HCl (pH 8.0), 60 mM MgCl₂, 4 mM ATP, 1 mM DTT, 0.02% (w/v) BSA, 4 mg/mL crude E. coli tRNA tRNA (Roche), 0.1 mM isoleucine and 5 mCi L-[4,5-3H]isoleucine (100 Ci/mmole, GE Healthcare) and 20% (v/v) DMSO for 1 hour at 30° C. The reaction was stopped by adding 10 μL of 10% (v/v) acetic acid followed by two acidic phenol (Sigma) extractions. The mischarged tRNA in the top aqueous phase was removed and precipitated by adding two volumes of 96% (v/v) ethanol and incubating at −20° C. for 30 minutes. The precipitate was pelleted by centrifugation at 13,200×g for 30 minutes and the mischarged tRNA pellet was washed twice with 70% (v/v) ethanol and then resuspended in 50 mM potassium phosphate buffer pH 5.2.

The reaction was terminated after 2 hours incubation at 30° C. by the addition of acetic acid to 0.17% (v/v). The isoleucylated crude tRNA^{Leu} was purified by extracting twice with acidic phenol-chloroform extractions (pH 4.3), followed by ethanol precipitation. The tRNA pellet was washed twice with 70% ethanol, dried and then resuspended in 50 mM potasium phosphate (pH 5.0) and stored at −20° C. An aliquot was precipitated with 10% (w/v) TCA to quantify ile-tRNA^{Leu}.

Post-transfer editing hydrolysis assays were carried out at 30° C. in 50 mM Hepes (pH 8), 10 mM MgCl₂, 30 mM KCl, with ³H-isoleucine-tRNA crude (˜0.3 μCi/mL). Each reaction was initiated by addition of the 150 nM enzyme. At each time point three 20 μL aliquots of the reaction mixture was added to 200 μL of 10% (w/v) TCA in a Millipore filter plate and precipitated for 20 minutes at 4° C. The precipitate was filtered and washed three times with 200 μL of 5% (w/v) TCA, then dried and 20 μL Supermix scintillation cocktail was added. The Millipore filter plates were counted in the MicroBeta Trilux. The IC₅₀ was determined by the amount of inhibitor that inhibited 50% activity, 100% post-transfer editing was calculated by taking the activity of the no enzyme control from the wild-type enzyme activity.

Compare the minimal inhibitory concentration (MIC) of a tolC Escherichia coli strain bearing a pUC derived plasmid with and without an leuS gene insert.

If the MIC of the strain bearing the extra copies of leuS is greater than 2-fold more than the control strain then pour LB agar plates with four times the concentration of the MIC of the compound.

Plate 1×10¹⁰ E. coli on ten plates containing 4×MIC of the compound. Incubate for 1-2 days at 37° C. and pick ten colonies and restreak on 4×MIC LB agar plates to confirm resistance.

Take one large colony from each of the ten E. coli resistant mutants and resuspend in 50 μL of PCR buffer.

Amplify the editing domain of CDC60 using a proof-reading PCR enzyme and the following primers, SEQ ID NO: 29, ggcaccgtggacgtacgacaacatcgc and SEQ ID NO: 30, gggaaacaccccagtcgcgcaggcgg.

Purify the 980 by PCR product using either Qiagen or Promega PCR cleanup kits.

Sequence amplify the mutant DNA and compared it to wild-type. If the mutant DNA bears mutations in the editing domain the inhibitor affects leucyl-tRNA synthetase via the editing domain.

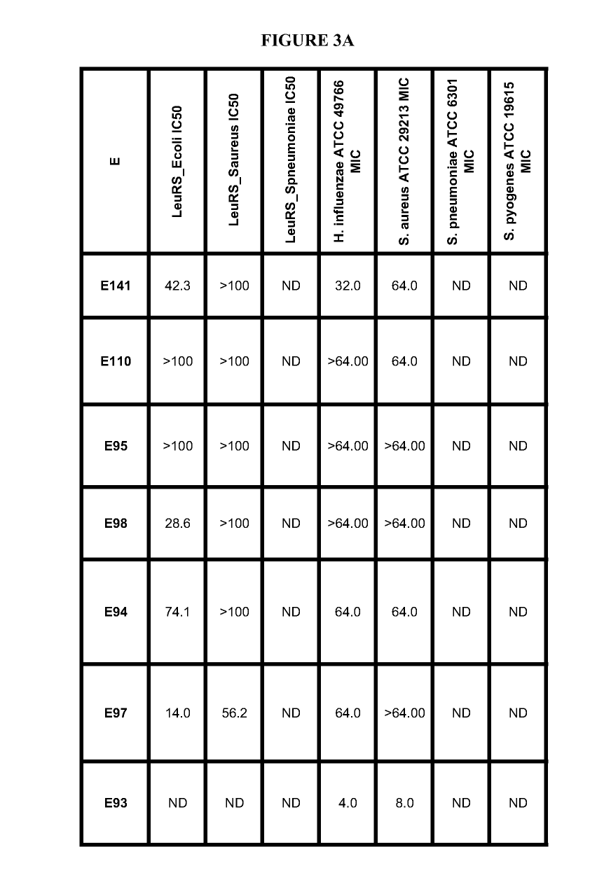

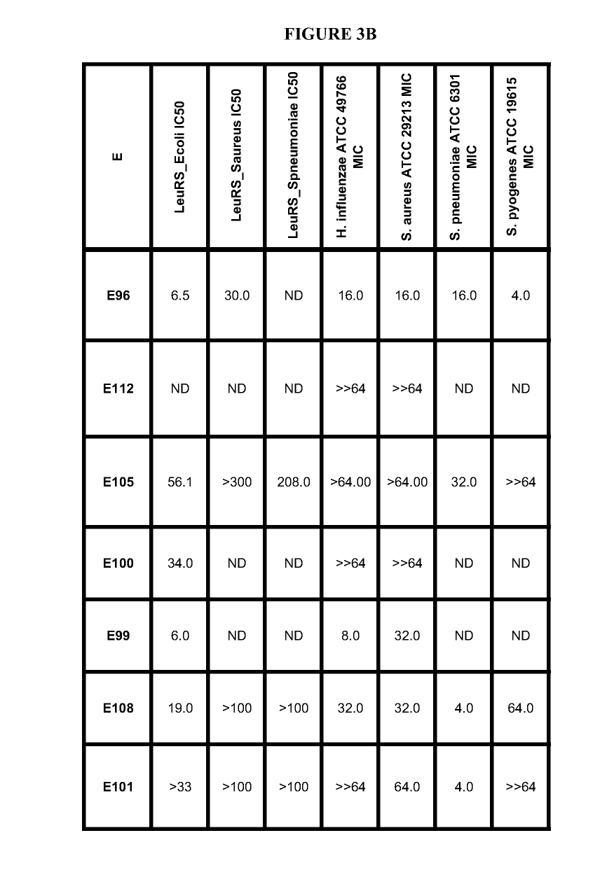

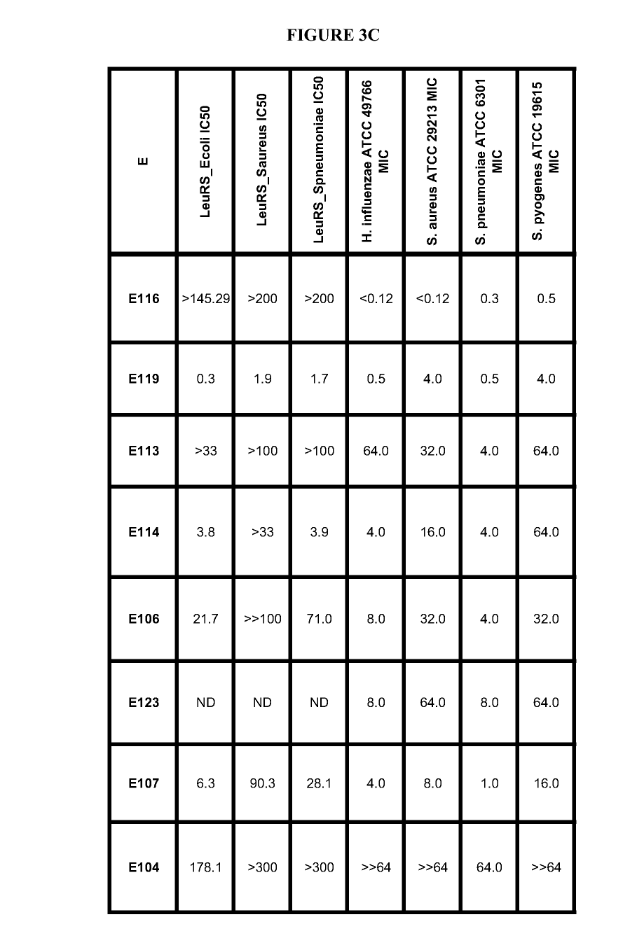

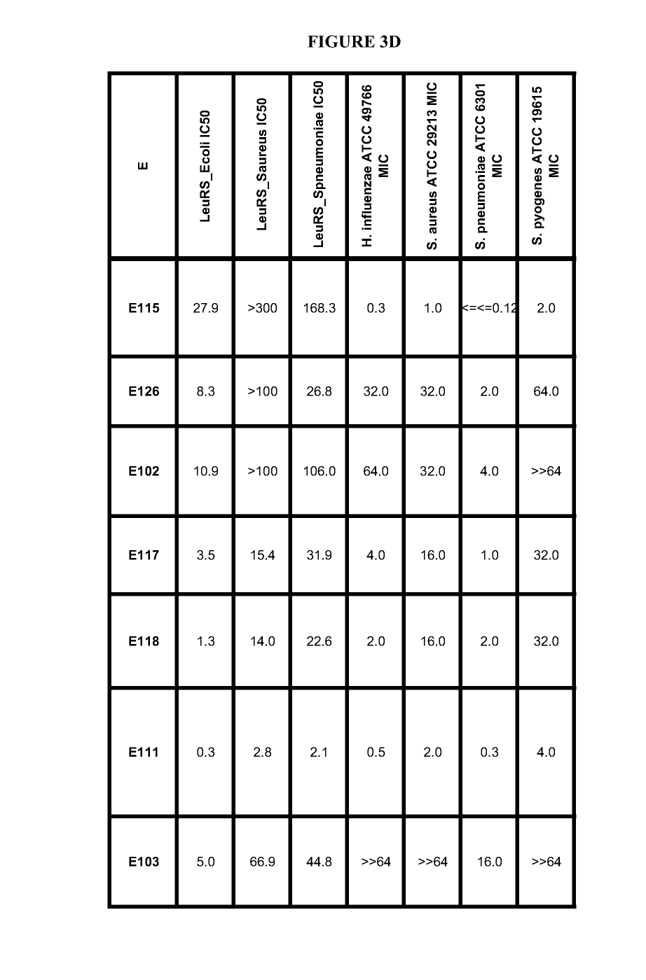

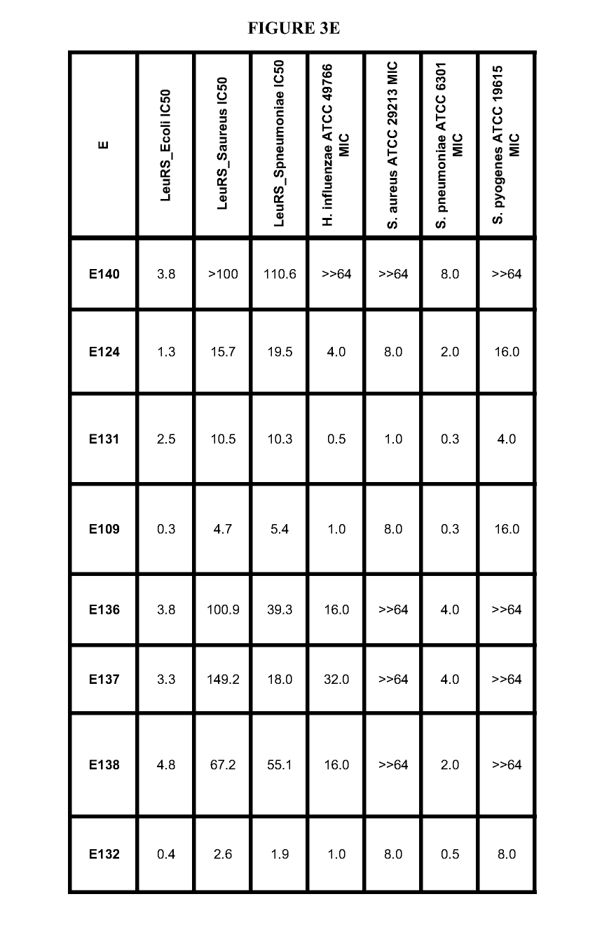

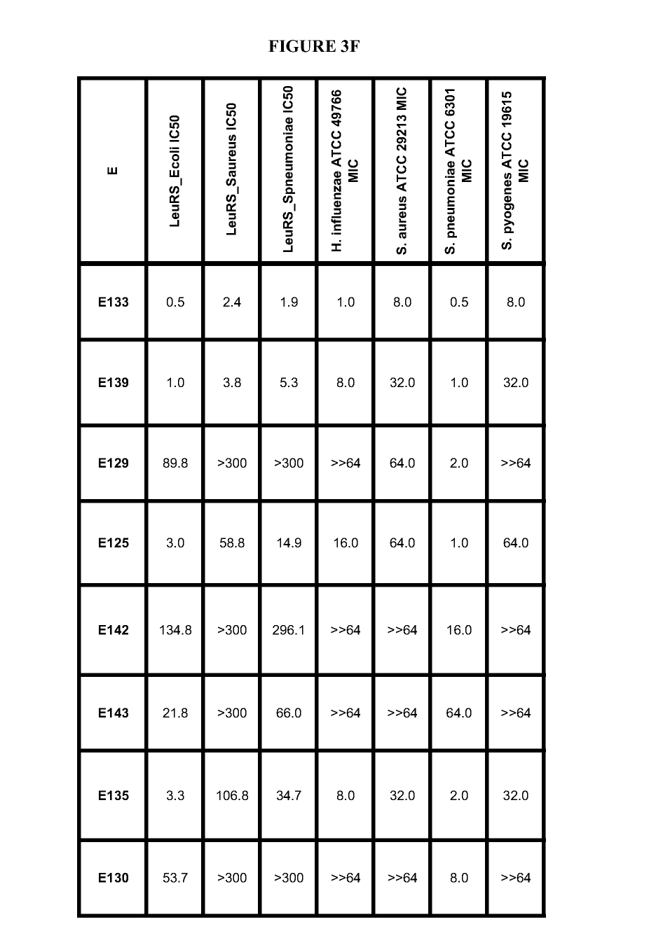

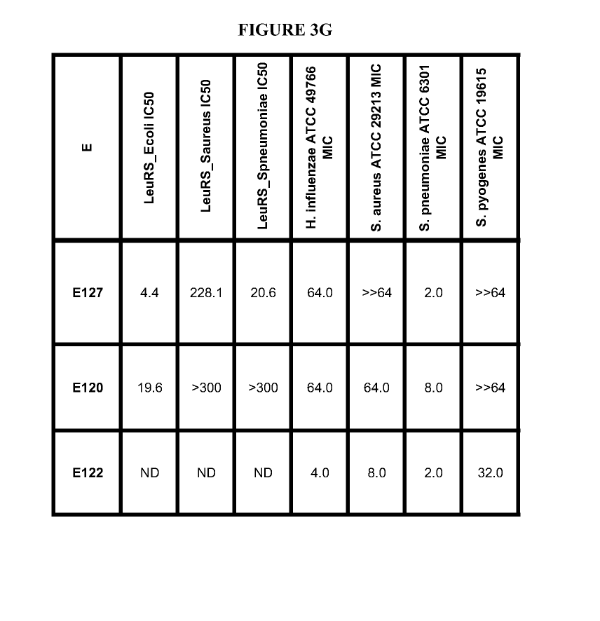

In vitro testing results for exemplary compounds of the invention are provided in FIG. 3.

### Example 2

### Antibacterial MIC Testing

All MIC testing of bacteria followed the Clinical and Laboratory Standards Institute (CLSI) guidelines for antimicrobial testing of aerobic bacteria (Methods for Dilution Antimicrobial Susceptibility Tests for Bacteria That Grow Aerobically; Approved Standard—Seventh Edition)(M07-A7) and anaerobic bacteria (Methods for Antimicrobial Susceptibility Testing of Anaerobic Bacteria; Approved Standard—Seventh Edition) (M11-A7). MIC data for exemplary compounds of the invention are provided in FIG. 2 and FIG. 3.

### Example 3

Anti-Inflammatory In vitro Assays

The ability of the compounds described herein to inhibit pro-inflammatory cytokines or phosphodiesterases can be tested.

### Cytokine Assay

Frozen human peripheral blood mononucleocytes (PBMC) were thawed and centrifuged. Cryopreservation media is aspirated off of the cell pellet, and the cells are resuspended in fresh culture media (CM) comprising RPMI 1640 and 10% FBS in 96 well plates. A compound described herein is dissolved in DMSO to form a 10 mM sample (DMSO, 100%). The 10 mM samples are diluted to 100 μM in CM (DMSO, 1%), then further diluted to 10, 1, 0.1, 0.01 μM in 200 μL of CM (n=3). Inducer (1 μg/mL LPS for TNF-α and IL-1β [and IL-6] or 20 μg/mL PHA for IFNγ, IL-2, IL-4, IL-5 and IL-10. IL-23 is induced with 100 ng/ml IFN-g+1 mg/ml LPS, using THP-1 cells. Vehicle (1% DMSO) is used as a control for this experiment. Vehicle without inducer are used as a negative control. Cells are incubated at 37° C., 5% CO₂. Supernatants are extracted at 24 hours (for TNF-α, IL-1β, IL-2, IL-6 and IFNγ) and 48 hours (for IL-4, IL-5, IL-10 and IL-23), and stored at −20° C. Supernatants are thawed and assayed for cytokine expression using the fluorochrome-labeled cytokine-specific beads and a BD FACSArray™. IL-23 is assayed using a commercial ELISA kit (R&D Systems).

### PDE Isoform Profilinz

Recombinant human phosphodiesterase (PDE) enzymes are expressed in a baculoviral system. The assay is a modification of the 2-step method of Thompson & Appleman (Biochem. 10:311-316, 1971), which is adapted for 96-well plate format. Stock solutions are prepared at 40 mM in 100% DMSO. Final [DMSO] was 5%. A compound described herein is tested by performing 1 in 4 serial dilutions at starting concentration of 100 mM. Each concentration is tested in duplicate. IC50s are generated from 11-point curves and analyzed using Prism software (GraphPad Inc.). PDE isoforms tested include PDE1A3 (cAMP), PDE1A3 (cGMP), PDE2A3, PDE3Cat, PDE4Cat, PDE4A4, PDE4B2, PDE4C2, PDE4D3, PDE5Cat, PDE6AB, PDE7A1, PDE8A1, PDE9A1, PDE10A1 (cAMP), PDE10A1 (cGMP), PDE11A1 (cAMP) and PDE11A1 (cGMP).

### PDE4 Assay

PDE4 partially purified from human U-937 myeloid leukemia cells is used.

A compound described herein and/or vehicle is incubated with 0.2 mg enzyme and 1 mM cAMP containing 0.01 mM [3H]cAMP in Tris buffer pH 7.5 for 20 minutes at 25° C. The reaction is terminated by boiling for 2 minutes and the resulting AMP is converted to adenosine by addition of 10 mg/ml snake venom nucleotidase and further incubation at 37° C. for 10 minutes. Unhydrolyzed cAMP is bound to AG1-X2 resin, and remaining [3H]Adenosine in the aqueous phase is quantitated by scintillation counting. A compound described herein is tested at 10, 3, 1, 0.3, 0.1, 0.03, 0.01, 0.003, and 0.001 μM for IC₅₀ determination.

### Example 4

Anti-Inflammatory in vivo Assays
1. In vivo Anti-Inflammation Activity in Phorbol Ester Induced Mouse Ear Edema Model

Phorbol 12-myristate 13-acetate (PMA, 5 μg in 20 μL of acetone) is applied topically to the anterior and posterior surfaces of the right ear to eight groups of CD-1 (Crl.) derived male mice of 5 each (weighing 22±2 g). A compound described herein and vehicle (acetone:ethano1/1:1, 20 μL/ear) are each applied to both ears topically 30 minutes before and 15 minutes after PMA challenge. Dexamethasone (1 mg/ear×2) is used as the positive control was administered topically to test animals using the same application schedule. Ear swelling is then measured by a Dyer model micrometer gauge at 6 hours after PMA application as an index of inflammation. Percent inhibition is calculated according to the formula: [(Ic−It)/Ic]×100%, where Ic and It refer to increase of ear thickness (mm) in control and treated mice, respectively. Percent inhibition of 30 percent or more in ear swelling is considered significant anti-inflammatory activity.

2. In vivo Anti-Inflammation Activity in Oxazolone Induced Mouse Ear Edema Model

Groups of 5 BALB/c male mice weighing 23±2 g were used. The preshaved abdomens of test animals are sensitized by application of 100 μL of 1.5% oxazolone solution dissolved in acetone. Seven days after the initial sensitization, compound described herein, as well as vehicle (acetone:ethano1/1:1, 20 μL/ear) are each administered topically to the anterior and posterior surfaces of the right ear 30 minutes before, and 15 minutes after, challenge by a second application of oxazolone (1% in acetone, 20 ml/ear) via topical route. As a positive control, indomethacin (0.3 mg/ear×2) is administered topically using the same treatment regime as for the test compounds. Twenty-four hours after the second application of oxazolone, the ear thickness of each mouse is measured with a Dyer model micrometer gauge. A 30 percent or more inhibition in ear swelling relative to the vehicle control is considered significant and indicated possible anti-inflammatory activity.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having a structure according to the formula: 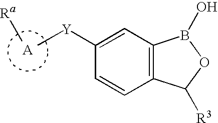
wherein
A is a member selected from cycloalkyl, heterocycloalkyl, aryl and heteroaryl;
Y is a member selected from O and —S(O)₂NH— wherein the sulfur in —S(O)₂NH— is covalently attached to A;
R³ is a member selected from H, cyano and substituted alkyl;
R^{a} is a member selected from H, —OR¹⁰, —NR¹⁰R¹¹, —SR¹⁰, —S(O)R¹⁰, —S(O)₂R¹⁰, —S(O)₂NR¹⁰R¹¹, —C(O)R¹⁰, —C(O)OR¹⁰, —C(O)NR¹⁰R¹¹, nitro, cyano, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl
wherein
each R¹⁰ and each R¹¹ is a member independently selected from H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl
with the proviso that R¹⁰ and R¹¹, together with the nitrogen to which they are attached, are optionally combined to form a 5- to 7-membered substituted or unsubstituted heterocycloalkyl ring;
with the proviso that when Y is O, R³ is a member selected from cyano and substituted alkyl;
with the proviso that when Y is —S(O)₂NH—, R³ is H, and R^{a} is not H or unsubstituted alkyl or halosubstituted alkyl
and salts thereof.

2. The compound of claim 1, having a structure according to the formula:

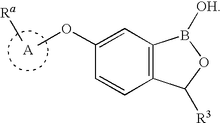

3. The compound of claim 2, having a structure which is a member selected from:

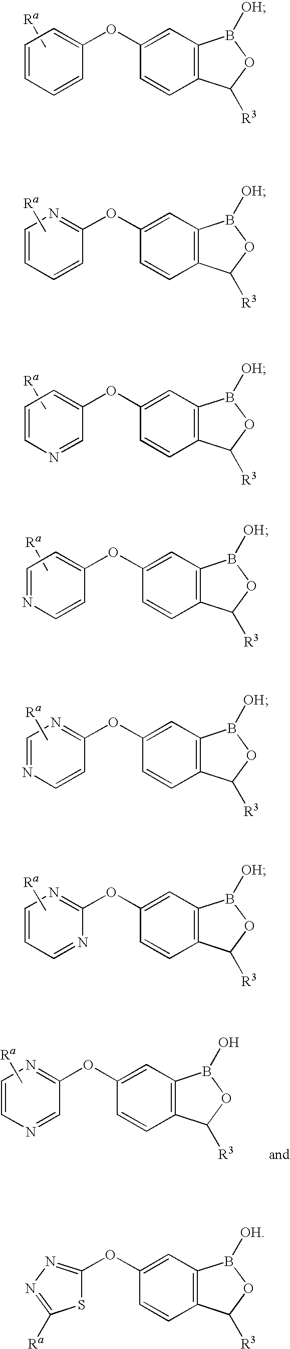

4. The compound of claim 3, wherein R^{a} is a member selected from H, F, Cl, —OR^{10a} and —C(O)OR^{10b},
wherein R^{10a} is alkyl, optionally substituted with a member selected from NH₂ and phenyl
wherein R^{10b} is unsubstituted alkyl.

5. The compound of claim 3, wherein R^{a} is —O(CH₂)ₙNH₂, wherein n is an integer selected from 1 to 6.

6. The compound of claim 5, wherein n is 2 or 3 or 4.

7. The compound of claim 2, having a structure according to the formula: 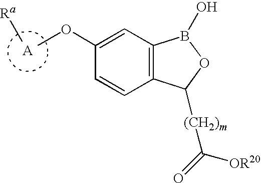
wherein m is an integer selected from 1 to 6 and R²⁰ is a member selected from H and unsubstituted alkyl.

8. The compound of claim 7, wherein m is 1 or 2 or 3.

9. The compound of claim 7, having a structure according to the formula:

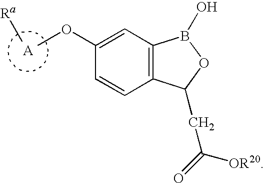

10. The compound of claim 9, wherein R²⁰ is H.

11. The compound of claim 9, wherein R²⁰ is C₁ or C₂ or C₃ unsubstituted alkyl.

12. The compound of claim 2, wherein R³ is —CH₂COOH or —CH₂COOCH₃ or —CH₂COOCH₂CH₃.

13. The compound of claim 9, having a structure according to the formula:

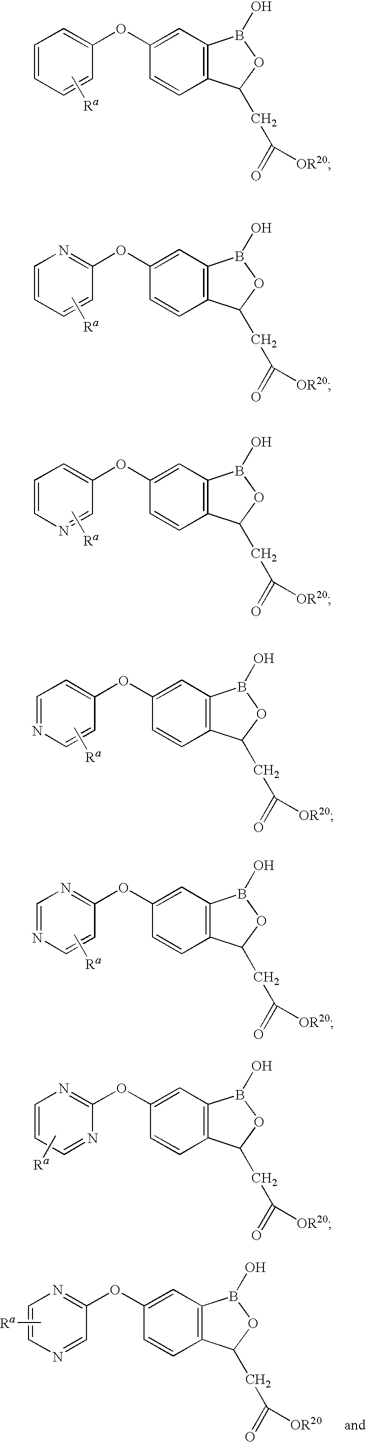

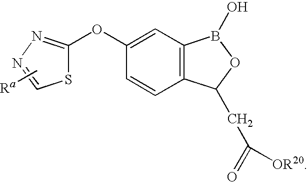

14. The compound of claim 1, having a structure according to the formula: 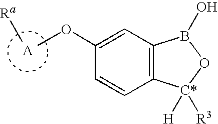
wherein C* is a carbon atom which is a stereocenter which has a configuration of (R) or (S).

15. The compound of claim 14, wherein C* is a stereocenter which has a (R) configuration.

16. The compound of claim 14, having a structure according to the formula: 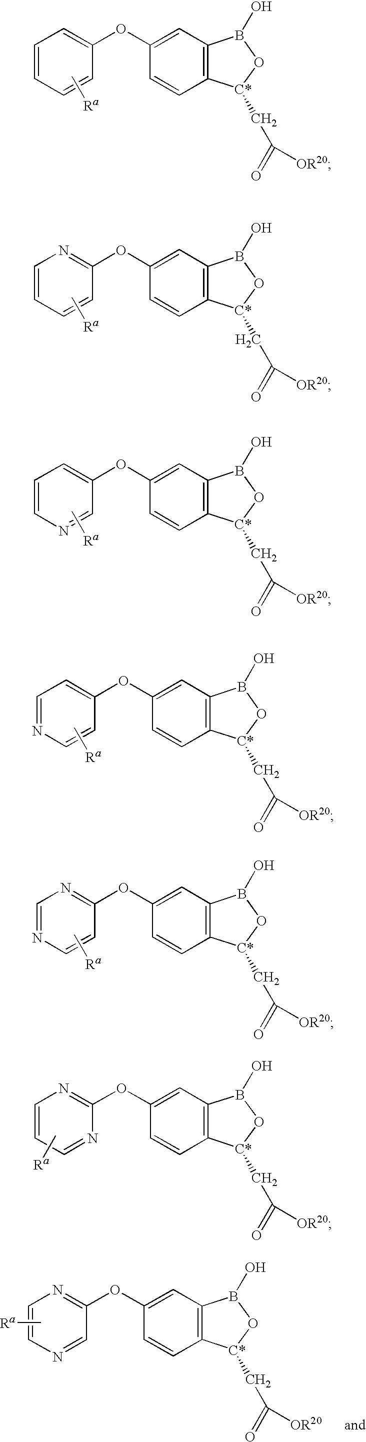 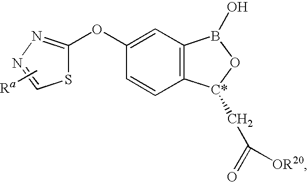
wherein R²⁰ is a member selected from H and unsubstituted alkyl.

17. The compound of claim 16, wherein R²⁰ is H.

18. The compound of claim 16, having a structure according to the formula: 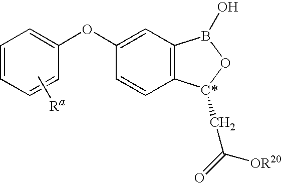
wherein R^{a} is —O(CH₂)ₙNH₂, wherein n is an integer selected from 1 to 6.

19. The compound of claim 18, which is:

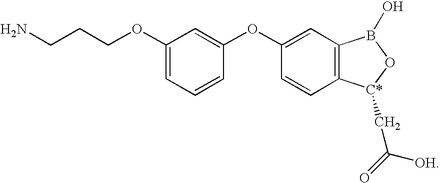

20. The compound of claim 1, having a structure according to the formula:

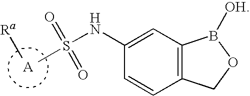

21. The compound of claim 20, wherein A is a member selected from phenyl, pyridinyl, furanyl, thiophenyl, pyrazolyl, imidazolyl, thiazolyl, triazolyl, and piperidinyl.

22. The compound of claim 20, wherein R^{a} is a member selected from cyano, nitro, aminoalkyl, hydroxyalkyl, —C(O)(CH₂)ₘ₁CH₃, —COOH, —C(O)O(CH₂)ₘ₁CH₃, —O(CH₂)ₘ₁CH₃, —O(CH₂)ₘ₁CF₃, —O(CH₂)ₘ₁CHF₂, —OH, —NH₂, —NHCH₃, —NHC(O)H, —NHC(O)(CH₂)ₘ₁CH₃, —NHOH, —NHS(O)₂NH₂, —NH₂S(O)₂CH₃, —S(O)₂CH₃,
wherein m1 is an integer which is a member selected from 0 to 3.

23. The compound of claim 21, having a structure according to the formula:

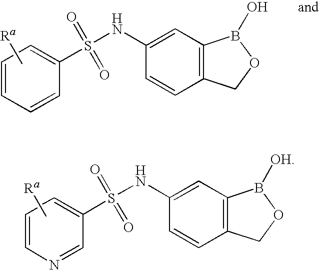

24. The compound of claim 23, having a structure according to the formula:

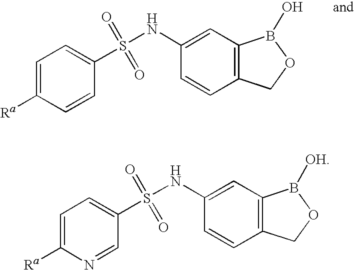

25. The compound of claim 24, wherein R^{a} is a member selected from OH and NH₂.

26. A combination comprising:
a) a compound of claim 1, or a pharmaceutically acceptable salt thereof; and
b) a therapeutically active agent.

27. The combination of claim 26, wherein said therapeutically active agent is an antibiotic which comprises a β-lactam moiety.

28. A pharmaceutical formulation comprising:
a) a compound of claim 1 or a combination of claim 26, or a pharmaceutically acceptable salt thereof; and
b) a pharmaceutically acceptable excipient.

29. The pharmaceutical formulation of claim 28, wherein said formulation is a unit dosage form.

30. The pharmaceutical formulation of claim 29, wherein said formulation is a member selected from an oral unit dosage form and a topical unit dosage form.

31. A method of treating a bacterial infection comprising:
administering to an animal suffering from said infection an effective amount of a compound of claim 1, or a pharmaceutically-acceptable salt thereof, and an effective amount of an antibiotic, or a pharmaceutically acceptable salt thereof, wherein said antibiotic comprises a β-lactam moiety, thereby treating the bacterial infection.

32. The method of claim 31, wherein a bacteria involved with said infection is resistant to said antibiotic.

33. The method of claim 31, wherein the antibiotic is a member selected from a penicillin, cephalosporin, monobactam, carbapenem and derivatives thereof.

34. The method of claim 33, wherein the antibiotic is a penicillin or derivatives thereof.

35. The method of claim 34, wherein said penicillin is a member selected from narrow spectrum penicillins, narrow spectrum penicillinase-resistant penicillins, narrow spectrum β-lactamase-resistant penicillins, moderate spectrum penicillins, broad spectrum penicillins and extended spectrum penicillins.

36. The method of claim 35, wherein said penicillin is a narrow spectrum penicillin which is a member selected from benzathine penicillin, benzylpenicillin (penicillin G), phenoxymethylpenicillin (penicillin V) and procaine penicillin.

37. The method of claim 35, wherein said penicillin is a narrow spectrum penicillinase-resistant penicillins which is a member selected from methicillin, dicloxacillin and flucloxacillin.

38. The method of claim 35, wherein said penicillin is a narrow spectrum β-lactamase-resistant penicillin which is temocillin.

39. The method of claim 35, wherein said penicillin is a moderate spectrum penicillin which is a member selected from amoxicillin and ampicillin.

40. The method of claim 35, wherein said penicillin is a broad spectrum penicillin which is a member selected from co-amoxiclav (amoxicillin and clavulanic acid).

41. The method of claim 35, wherein said penicillin is an extended spectrum penicillin, which is a member selected from azlocillin, carbenicillin, ticarcillin, mezlocillin and piperacillin.

42. The method of claim 31, wherein the antibiotic is a cephalosporin or a derivative thereof.

43. The method of claim 42, wherein the cephalosporin is a member selected from a first-generation cephalosporin, second-generation cephalosporin, second-generation cephamycin, third-generation cephalosporin and fourth-generation cephalosporin.

44. The method of claim 42, wherein the cephalosporin is a member selected from cefalexin, cephalothin and cefazolin.

45. The method of claim 42, wherein the cephalosporin is a member selected from cefaclor, cefuroxime and cefamandole.

46. The method of claim 42, wherein the cephalosporin is a member selected from cefotetan and cefoxitin.

47. The method of claim 42, wherein the cephalosporin is a member selected from ceftriaxone, cefotaxime, cefpodoxime and ceftazidime.

48. The method of claim 42, wherein the cephalosporin is a member selected from cefepime and cefpirome.

49. The method of claim 31, wherein the antibiotic is a monobactam.

50. The method of claim 49, wherein the monobactam is aztreonam.

51. The method of claim 31, wherein the antibiotic is a carbapenem.

52. The method of claim 51, wherein the carbapenem is a member selected from imipenem, cilastatin, meropenem, ertapenem and faropenem.

53. The method of claim 31, wherein said animal is a human.

54. A method of killing or inhibiting the growth of a bacteria, said method comprising:
contacting said bacteria with an effective amount of a compound of claim 1 or a combination of claim 26, or a pharmaceutically acceptable salt thereof, thereby killing or inhibiting the growth of the bacteria.

55. The method of claim 54, further comprising contacting said bacteria with an effective amount of an antibiotic, or a pharmaceutically acceptable salt thereof, wherein said antibiotic comprises a β-lactam moiety.

56. The method of claim 55, wherein the bacteria is resistant to said antibiotic.

57. A method of inhibiting a β-lactamase, comprising contacting the β-lactamase with an effective amount of a compound of claim 1, or a pharmaceutically acceptable salt thereof, thereby inhibiting the β-lactamase.

58. The method of claim 57, wherein the β-lactamase is a member selected from a Group 1 β-lactamase, a Group 2 β-lactamase, a Group 3 β-lactamase, and a Group 4 β-lactamase.

59. The method of claim 58, wherein said Group 1 β-lactamase is a cephalosporinase.

60. The method of claim 58, wherein said Group 2 β-lactamase is a member selected from penicillinase, a Group 2b, Group 2be, Group 2br, carbenicillinase, cloxacilanase, cephalosporinase and carbapenamase.

61. The method of claim 58, wherein said Group 3 β-lactamase is a metallo-β-lactamase.

62. The method of claim 58, wherein said Group 4 β-lactamase is a penicillinase.

63. The method of claim 57, wherein the β-lactamase is a member selected from a class A β-lactamase, a class B β-lactamase, a class C β-lactamase, and a class D β-lactamase.

64. The method of claim 63, wherein the class A β-lactamase is a member selected from a TEM β-lactamase, SHV β-lactamase, CTX-M β-lactamase and a KPC β-lactamase.

65. The method of claim 63, wherein the class C β-lactamase is a member selected from a CMY β-lactamase and a AmpC β-lactamase.

66. The method of claim 63, wherein the class D β-lactamase is an OXA β-lactamase.

67. The method of claim 63, wherein the β-lactamase is a metallo β-lactamase.

68. The method of claim 63, wherein the metallo β-lactamase is a member selected from an IMP carbapenemase and a VIM β-lactamase.

69. The method of claim 57, wherein the contacting takes place in vitro.

70. A method of treating a bacterial infection comprising: administering to an animal suffering from said infection an effective amount of a compound of claim 1, or a pharmaceutically-acceptable salt thereof, thereby treating the bacterial infection.

71. A method of inhibiting the editing domain of a t-RNA synthetase, comprising: contacting the synthetase with an effective amount of a compound of claim 1, or a pharmaceutically-acceptable salt thereof, thereby inhibiting the synthetase.

72. The method of claim 71, wherein the synthetase is a leucyl t-RNA synthetase.

73. The use of a compound of claim 1 or a combination of claim 26, or a pharmaceutically acceptable salt thereof, in the manufacture of a medicament for the treatment and/or prophylaxis of bacterial infection.


SEQUENCE LISTING (30 sequences)

SEQ ID NO: 1 - DNA, 85 bp, Saccharomyces cerevisiae
gggagtttggccgagtggtttaaggcgtcagatttaggctctgatatcttcggatgcaagggttcgaatcccttagctctcacca

SEQ ID NO: 2 - DNA, 77 bp, Saccharomyces cerevisiae
gaaactataattcaattggttagaatagtattttgataaggtacaaatataggttcaatccctgttagtttcatcca

SEQ ID NO: 3 - protein, 258 aa, Artificial Sequence
  Polypeptide of editing domain of tRNA synthetase
TPQEYIGVKIEALEFADDAAKIIDSSSDLDKSKKFYFVAATLRPETMYGQTCCFVSPTIEYGIFDAGDSYFITTERAFKNMSYQKLTPKRGFYKPIVTVPGKAFIGTKIHAPQSVYPELRILPMETVIATKGTGVVTCVPSNSPDDYITTKDLLHKPEYYGIKPEWIDHEIVPIMHTEKYGDLTAKAIVEEKKIQSPKDKNLLAEAKKIAYKEDYYTGTMIYGPYKGEKVEQAKNKVKADMIAAGEAFVYNEPESQDP

SEQ ID NO: 4 - protein, 280 aa, Artificial Sequence
  Polypeptide of editing domain of tRNA synthetase
MTPQEYIGVKIEALEFADDAAKIIDSSSDLDKSKKFYFVAATLRPETMYGQTCCFVSPTIEYGIFDAGDSYFITTERAFKNMSYQKLTPKRGFYKPIVTVPGKAFIGTKIHAPQSVYPELRILPMETVIATKGTGVVTCVPSNSPDDYITTKDLLHKPEYYGIKPEWIDHEIVPIMHTEKYGDLTAKAIVEEKKIQSPKDKNLLAEAKKIAYKEDYYTGTMIYGPYKGEKVEQAKNKVKADMIAAGEAFVYNEPESQDPQDPNSSSVDKLAAALEHHHHH

SEQ ID NO: 5 - protein, 303 aa, Artifical Sequence
  Polypeptide of editing domain of tRNA synthetase
TCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWG

SEQ ID NO: 6 - protein, 308 aa, Artificial Sequence
  Polypeptide of editing domain of tRNA synthetase
TCKPDYYRWEQWLFTRLFEKGVIYRKNGTVNWDPADQTVLANEQVIDGRGWRSGALIEKREIPMYYFRITDYADELLESLDELPGWPEQVKTMQRNWIGKSRGMEVQFPYDQASIGHEGTLKVFTTRPDTLMGATYVAVAAEHPLATQAAQGNAALQAFIDECKSGSVAEADMATQEKKGMATSLFVEHPLTGEKLPVWVANYVLMHYGDGAVMAVPAHDERDFEFAHKYNLPVKAVVRTSAGDDVGSEWLAAYGEHGQLINSGEFDGLDFQGAFDAIEAALIRKDLGKSRTQFRLRDWGISRQRYWG

SEQ ID NO: 7 - protein, 298 aa, Artificial Sequence
  Polypeptide of editing domain of tRNA synthetase
TTDPEYYKWTQWIFIQLYNKGLAYVDEVAVNWCPALGTVLSNEEVIDGVSERGGHPVYRKPMKQWVLKITEYADQLLADLDDLDWPESLKDMQRNWIGRSEGAKVSFDVDNTEGKVEVFTTRPDTIYGASFLVLSPEHALVNSITTDEYKEKVKAYQTEASKKSDLERTDLAKDKSGVFTGAYAINPLSGEKVQIWIADYVLSTYGTGAIMAVPAHDDRDYEFAKKFDLLIIEVIEGGNVEEAAYTGEGKHINSGELDGLENEAAITKAIQLLEQKGAGEKKVYKLRDWLFSRQRYWG

SEQ ID NO: 8 - protein, 192 aa, Escherichia coli
GRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYR

SEQ ID NO: 9 - protein, 197 aa, Pseudomonas
GKSRGMEVQFPYDQASIGHEGTLKVFTTRPDTLMGATYVAVAAEHPLATQAAQGNAALQAFIDECKSGSVAEADMATQEKKGMATSLFVEHPLTGEKLPVWVANYVLMHYGDGAVMAVPAHDERDFEFAHKYNLPVKAVVRTSAGDDVGSEWLAAYGEHGQLINSGEFDGLDFQGAFDAIEAALIRKDLGKSRTQFR

SEQ ID NO: 10 - protein, 188 aa, Staphylococcus aureus
GRSEGAKVSFDVDNTEGKVEVFTTRPDTIYGASFLVLSPEHALVNSITTDEYKEKVKAYQTEASKKSDLERTDLAKDKSGVFTGAYAINPLSGEKVQIWIADYVLSTYGTGAIMAVPAHDDRDYEFAKKFDLLIIEVIEGGNVEEAAYTGEGKHINSGELDGLENEAAITKAIQLLEQKGAGEKKVYK

SEQ ID NO: 11 - protein, 860 aa, Escherichia coli [sequence omitted]

SEQ ID NO: 12 - protein, 868 aa, Pseudomonas
MHEQYTPRDVEAAAQNAWDEQQSFAVTEQPGKETYYCLSMFPYPSGKLHMGHVRNYTIGDVIARYQRMLGKNVLQPMGWDAFGMPAENAAMKNNVAPAKWTYENIDYMKTQLKSLGLAIDWSREVTTCKPDYYRWEQWLFTRLFEKGVIYRKNGTVNWDPADQTVLANEQVIDGRGWRSGALIEKREIPMYYFRITDYADELLESLDELPGWPEQVKTMQRNWIGKSRGMEVQFPYDQASIGHEGTLKVFTTRPDTLMGATYVAVAAEHPLATQAAQGNAALQAFIDECKSGSVAEADMATQEKKGMATSLFVEHPLTGEKLPVWVANYVLMHYGDGAVMAVPAHDERDFEFAHKYNLPVKAVVRTSAGDDVGSEWLAAYGEHGQLINSGEFDGLDFQGAFDAIEAALIRKDLGKSRTQFRLRDWGISRQRYWGCPIPIIHCPSCGDVPVPEDQLPVTLPENVVPDGAGSPLARMPEFYECTCPKCGTAAKRETDTMDTFVESSWYFARYASPNYDKGLVDPKAANHWLPVDQYIGGIEHAILHLLYARFFHKLMRDEGLVTSNEPFKNLLTQGMVVAETYYRVASNGGKDWFNPADVEIERDAKAKIIGARLKTDGLPVEIGGTEKMSKSKNNGVDPQSMIEQYGADTCRLFMMFASPPDMSLEWSDSGVEGASRFLRRVWRLAQAHVAQGLPGQLDIAALSDEQKVIRRAIHAAIKQASTDVGQFHKFNTAIAQVMTVMNVLEKAPQVTAQDRALLQEGLEAVTLLLAPITPHISHELWKQLGHEQAVIDATWPSVDESALVQDTVTLVVQVNGKLRGQVEMPAAASREEIEAAARNNENVLRFTDGLTIRKVIVVPGKLVNIVAN

SEQ ID NO: 13 - protein, 804 aa, Staphylococcus aureus
MNYNHNQIEKKWQDYWDENKTFKTNDNLGQKKFYALDMFPYPSGAGLHVGHPEGYTATDIISRYKRMQGYNVLHPMGWDAFGLPAEQYALDTGNDPREFTKKNIQTFKRQIKELGFSYDWDREVNTTDPEYYKWTQWIFIQLYNKGLAYVDEVAVNWCPALGTVLSNEEVIDGVSERGGHPVYRKPMKQWVLKITEYADQLLADLDDLDWPESLKDMQRNWIGRSEGAKVSFDVDNTEGKVEVFTTRPDTIYGASFLVLSPEHALVNSITTDEYKEKVKAYQTEASKKSDLERTDLAKDKSGVFTGAYAINPLSGEKVQIWIADYVLSTYGTGAIMAVPAHDDRDYEFAKKFDLLIIEVIEGGNVEEAAYTGEGKHINSGELDGLENEAAITKAIQLLEQKGAGEKKVNYKLRDWLFSRQRYWGEPIPVIHWEDGTMTTVPEEELPLLLPETDEIKPSGTGESPLANIDSFVNVVDEKTGMKGRRETNTMPQWAGSCWYYLRYIDPKNENMLADPEKLKHWLPVDLYIGGVEHAVLHLLYARFWHKVLYDLGIVPTKEPFQKLFNQGMILGEGNEKMSKSKGNVINPDDIVQSHGADTLRLYEMFMGPLDAAIAWSEKGLDGSRRFLDRVWRLIVNEDGTLSSKIVTTNNKSLDKVYNQTVKKVTDDFETLGFNTAISQLMVFINECYKVDEVYKPYIEGFVKMLAPIAPHIGEELWSKLGHEESITYQPWPTYDEALLVDDEVEIVVQVNGKLRAKIKIAKDTSKEEMQEIALSNDNVKASIEGKDIMKVIAVPQKLVNIVAK

SEQ ID NO: 14 - DNA, 87 bp, E. coli
gcgaaggtggcggaattggtagacgcgctagcttcaggtgttagtgtccttacggacgtgggggttcaagtcccccccctcgcacca

SEQ ID NO: 15 - DNA, 85 bp, E. coli
gcgggagtggcgaaattggtagacgcaccagatttaggttctggcgccgcaaggtgtgcgagttcaagtctcgcctcccgcacca

SEQ ID NO: 16 - DNA, 85 bp, E. coli
gccgaagtggcgaaatcggtagacgcagttgattcaaaatcaaccgtagaaatacgtgccggttcgagtccggccttcggcacca

SEQ ID NO: 17 - DNA, 87 bp, E. coli
gccgaggtggtggaattggtagacacgctaccttgaggtggtagtgcccaatagggcttacgggttcaagtcccgtcctcggtacca

SEQ ID NO: 18 - DNA, 87 bp, E. coli
gcccggatggtggaatcggtagacacaagggatttaaaatccctcggcgttcgcgctgtgcgggttcaagtcccgctccgggtacca

SEQ ID NO: 19 - DNA, 88 bp, E. coli
  modified_base 8: s4u
gcccggaugguggaaucgguagacacaagggayunaaaaaycccucggcguucgcgcugugcgggtycaagucccgcuccggguacca

SEQ ID NO: 20 - DNA, 88 bp, E. coli
  modified_base 18: gm
gcgaagguggcggaauugguagacgcgcuagcuucagngygyuaguguccuuacggacgugggggtycaaguccccccccucgcacca

SEQ ID NO: 21 - DNA, 88 bp, E. coli
  modified_base 18: gm
gccgaggugguggaauugguagacacgcuaccuugagngygguagugcccaauagggcuuacgggtycaagucccguccucgguacca

SEQ ID NO: 22 - DNA, 85 bp, Pseudomonas aeruginosa
gcggacgtggtggaattggtagacacactggatttaggttccagcgccgcaaggcgtgagagttcgagtctctccgtccgcacca

SEQ ID NO: 23 - DNA, 89 bp, Staphylococcus aureus
gccggggtggcggaactggcagacgcacaggacttaaaatcctgcggtgagagatcaccgtaccggttcgattccggtcctcggcacca

SEQ ID NO: 24 - DNA, 89 bp, Staphylococcus aureus
  gene (0)...(0)
gccggggtggcggaactggcagacgcacaggacttaaaatcctgcggtgagtgatcaccgtaccggttcgattccggtcctcggcacca

SEQ ID NO: 25 - DNA, 1155 bp, Artifical Sequence
  gene encoding CMY-2 beta-lactamase
catatgatgaaaaaatcgttatgctgcgctctgctgctgacagcctctttctccacatttgctgccgcaaaaacagaacaacagattgccgatatcgttaatcgcaccatcaccccgttgatgcaggagcaggctattccgggtatggccgttgccgttatctaccagggaaaaccctattatttcacctggggtaaagccgatatcgccaataaccacccagtcacgcagcaaacgctgtttgagctaggatcggttagtaagacgtttaacggcgtgttgggcggcgatgctatcgcccgcggcgaaattaagctcagcgatccggtcacgaaatactggccagaactgacaggcaaacagtggcagggtatccgcctgctgcacttagccacctatacggcaggcggcctaccgctgcagatccccgatgacgttagggataaagccgcattactgcatttttatcaaaactggcagccgcaatggactccgggcgctaagcgactttacgctaactccagcattggtctgtttggcgcgctggcggtgaaaccctcaggaatgagttacgaagaggcaatgaccagacgcgtcctgcaaccattaaaactggcgcatacctggattacggttccgcagaacgaacaaaaagattatgcctggggctatcgcgaagggaagcccgtacacgtttctccgggacaacttgacgccgaagcctatggcgtgaaatccagcgttattgatatggcccgctgggttcaggccaacatggatgccagccacgttcaggagaaaacgctccagcagggcattgcgcttgcgcagtctcgctactggcgtattggcgatatgtaccagggattaggctgggagatgctgaactggccgctgaaagctgattcgatcatcaacggcagcgacagcaaagtggcattggcagcgcttcccgccgttgaggtaaacccgcccgcccccgcagtgaaagcctcatgggtgcataaaacgggctccactggtggatttggcagctacgtagccttcgttccagaaaaaaaccttggcatcgtgatgctggcaaacaaaagctatcctaaccctgtccgtgtcgaggcggcctggcgcattcttgaaaagctgcaataagtcgac

SEQ ID NO: 26 - DNA, 891 bp, Artificial Sequence
  gene encoding KPC-2 beta lactamase
catatgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaagtcgac

SEQ ID NO: 27 - DNA, 867 bp, Artificial Sequence
  gene ecoding TEM-64 beta lactamase
catatgagtattcaacatttccgtgtcgcccttattccgttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgcccggaagaacgttttccaatgatgagcacttttaaagttctgctgtgtggcgcggtattatcccgtgttgacgccgggcaagagcaactcggtcgccgcattcactattctcagaatgacttggttaagtactcaccagtcacagaaaagcatcttacggatggcatgacagtacgcgaattatgcagtgctgccattaccatgagtgataacactgcggccaacttacttctgacaacgatcggcggcccgaaggagctgaccgcttttttgcacaacatgggggatcatgtaactcgccttgatagctgggaaccggagctgaatgaagccattccaaacgacgagcgtgacaccacgacccctgcagcaatggcaacaacgttgcgcaaactgttaactggcgaactgcttactctggcttcccggcaacaattaattgactggatggaggcggataaagttgcaggcccacttctgcgctcggcccttccggctggctggtttattgctgataaatctggcgccggtgagcgtgggtctcgcggtatcattgcagcactggggccagatggtaagccgtcccgtatcgtagttatctacacgacggggagtcaggcaactatggatgaacgaaatcgccagatcgctgagattggtgcctcactgattaagcattggctcgag

SEQ ID NO: 28 - DNA, 870 bp, Artificial Sequence
  gene encoding SHV-18 beta-lactamase
catatgatgaaaaaatcgttatgctgcgctctgctgctgacagcctctttctccacatttgctgccagcccgcagccgcttgagcaaattaaactaagcgaaagccagctgtcgggcagcgtaggcatgatagaaatggatctggccagcggccgcacgctgaccgcctggcgcgccgatgaacgctttcccatgatgagcacctttaaagtagtgctctgcggcgcagtgctggcgcgggtggatgccggtgacgaacagctggagcgaaagatccactatcgccagcaggatctggtggactactcgccggtcagcgaaaaacaccttgccgacggcatgacggtcggcgaactctgtgccgccgccattaccatgagcgataacagcgccgccaatctgctgctggccaccgtcggcggccccgcaggattgactgcctttttgcgccagatcggcgacaacgtcacccgccttgaccgctgggaaacggaactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgatcgggtcgccggaccgttgatccgctccgtgctgccggcgggctggtttatcgccgataagaccggagctgccaaacggggtgcgcgcgggattgtcgccctgcttggcccgaataacaaagcagagcggattgtggtgatttatctgcgggatacgccggcgagcatggccgagcgaaatcagcaaatcgccgggatcggcgcggcgctgatcgagcactggcaacgctaactcgag

SEQ ID NO: 29 - DNA, 27 bp, Artificial Sequence
  Primer
ggcaccgtggacgtacgacaacatcgc

SEQ ID NO: 30 - DNA, 26 bp, Artificial Sequence
  Primer
gggaaacaccccagtcgcgcaggcgg